(19) United States
(12) Patent Application Publication
(10) Pub. No.: US 2023/0416256 A1
(43) Pub. Date: Dec. 28, 2023

(54) SMALL MOLECULE INHIBITORS OF NF-kB INDUCING KINASE

(71) Applicant: Janssen Pharmaceutica NV, Beerse (BE)

(72) Inventors: William M. Jones, Jenkintown, PA (US); Alec D. Lebsack, Ladera Ranch, CA (US); Ronald L. Wolin, San Diego, CA (US); Alexander R. Rovira, San Diego, CA (US)

(73) Assignee: Janssen Pharamceutica NV, Beerse (BE)

(21) Appl. No.: 18/034,890

(22) PCT Filed: Nov. 11, 2021
(86) PCT No.: PCT/US2021/072343

§ 371 Date: May 2, 2023

Related U.S. Application Data
(60) Provisional application No. 63/113,117, filed on Nov. 12, 2020.

(51) Int. Cl. C07D 487/04; C07D 471/04; C07D 519/00

(52) U.S. Cl. CPC C07D 487/04; C07D 471/04; C07D 519/00

(57) ABSTRACT

This invention relates to compounds of Formula I that inhibit NIK and pharmaceutical compositions comprising such compounds and methods of using the same. These compounds and pharmaceutical compositions are useful for preventing or treating diseases such as cancer (such as B-cell malignancies including leukemias, lymphomas and myeloma), inflammatory disorders, autoimmune disorders, immunodermatologic disorders such as palmoplantar pustulosis and hidradenitis suppurativa, and metabolic disorders such as obesity and diabetes.

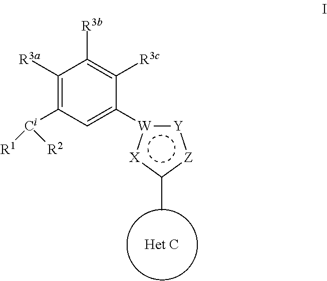

### FIELD

The present invention relates to the field of chemistry and medicine. More particularly, the present invention relates to NF-κB-inducing kinase inhibitors and their use in medical treatment.

### BACKGROUND

NF-κB INDUCING KINASE (NIK) is a serine/threonine kinase transcription factor propitiating the expression of various genes involved in immune response disorders, cell proliferation disorders, adhesion, apoptosis, and carcinogenesis. Because of this immune system regulatory role, inhibition of NIK blocks several downstream pathways that produce inflammatory molecules. Clinical validation with biologics has confirmed a key role for several NIK dependent pathways in autoimmune diseases. See, e.g., S. V. Navarra, et al., The Lancet, 2011; 377(9767):721-31. NIK plays a role propitiating immune response disorders, cell proliferation disorders, adhesion, apoptosis, and carcinogenesis, and one way to mitigate or eliminate the adverse effect associated with such increase is NIK inhibition. Therefore, there is a need to develop effective NIK inhibitors that can be used to treat various diseases.

### BRIEF SUMMARY

Some embodiments relate to a compound of Formula I or a pharmaceutically acceptable salt thereof,

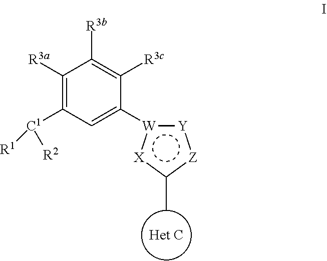

wherein
C¹ is —C(OH)—,
R¹ is C₁-C₃ alkyl;
R² is a 5 or 6 membered substituted or unsubstituted heteroaryl;
or R¹ and R² can be taken together with the carbon atom to which they are attached to form a substituted or unsubstituted heterocycle;
R^{3a}, R^{3b} and R^{3c} are independently H or C₁-C₃ alkyl;

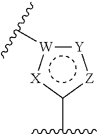

is a substituted or unsubstituted heteroaryl,
X, Y and Z are independently selected from O, S, N, N—R⁴ or C—R⁵;
W is N or C;
R⁴ is H or —C₁-C₃ alkyl; and
R⁵ is H, halo, CN, —C₁-C₃ alkyl or CH₂OH;
Het C is a substituted or unsubstituted 5-10 membered heteroaryl having at least one nitrogen atom.

In some embodiments, Het C is a substituted or unsubstituted 9 membered heteroaryl having at least two nitrogen atoms.

In some embodiments, Het C is selected from the group consisting of

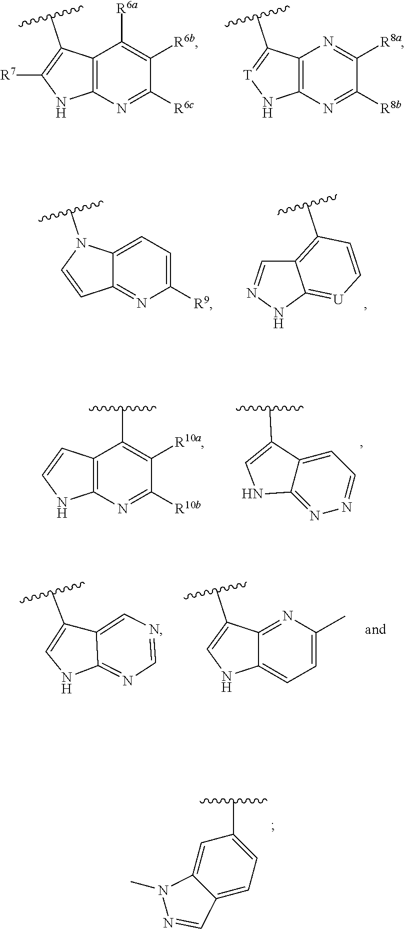

Wherein R^{6a}, R^{6b}, R^{6c} is independently H, halo, —C₁-C₃ alkyl, —C₁-C₃ haloalkyl, —CN, —CO₂H, —OC₁-C₃ alkyl, —SO₂CH₃ or —NH₂;
R⁷ is H, D, halo or —C₁-C₃ alkyl;
R^{8a}, R^{8b}, and R^{8c} is independently H, D, halo or —C₁-C₃ alkyl;
R⁹, R^{10a} and R^{10b} is independently H or NH₂;
T is N or C—R^{8c}; and
U is N or CH.

In some embodiments, the absolute stereochemistry of the carbon atom of C¹ is (R). In some embodiments, the absolute stereochemistry of the carbon atom of C¹ is (S).

Embodiments of this invention include the following compounds, and pharmaceutically acceptable salts thereof:
(S)-7-(3-(2-(1H-Pyrrolo[2,3-b]pyridin-3-yl)thiazol-4-yl)phenyl)-6,7-dihydro-5H-cyclopenta[b]pyridin-7-ol;
(R)-7-(3-(2-(1H-Pyrrolo[2,3-b]pyridin-3-yl)thiazol-4-yl)phenyl)-6,7-dihydro-5H-cyclopenta[b]pyridin-7-ol;
(R)-3-(3-(2-(1H-Pyrrolo[2,3-b]pyridin-3-yl)thiazol-5-yl)phenyl)-3-hydroxy-1-methylpyrrolidin-2-one;
(S)-3-(3-(2-(1H-Pyrrolo[2,3-b]pyridin-3-yl)thiazol-5-yl)phenyl)-3-hydroxy-1-methylpyrrolidin-2-one;
(R)-7-(3-(2-(7H-Pyrrolo[2,3-d]pyrimidin-5-yl)thiazol-4-yl)phenyl)-6,7-dihydro-5H-cyclopenta[b]pyridin-7-ol;
(S)-7-(3-(2-(7H-Pyrrolo[2,3-d]pyrimidin-5-yl)thiazol-4-yl)phenyl)-6,7-dihydro-5H-cyclopenta[b]pyridin-7-ol;
(R)-3-Hydroxy-1-methyl-3-(3-(2-(5-methyl-1H-pyrrolo[2,3-b]pyridin-3-yl)thiazol-4-yl)phenyl)pyrrolidin-2-one;
(R)-3-(3-(4-(1H-Pyrrolo[2,3-b]pyridin-3-yl)thiazol-2-yl)phenyl)-3-hydroxy-1-methylpyrrolidin-2-one;
(R)-7-(3-(2-(1H-Pyrrolo[2,3-b]pyridin-3-yl)thiazol-4-yl)phenyl)-6,7-dihydro-5H-pyrrolo[1,2-a]imidazol-7-ol;
(R)-7-(3-(4-(5H-Pyrrolo[2,3-b]pyrazin-7-yl)thiazol-2-yl)phenyl)-6,7-dihydro-5H-pyrrolo[1,2-a]imidazol-7-ol;
(S)-7-(3-(4-(5H-Pyrrolo[2,3-b]pyrazin-7-yl)thiazol-2-yl)phenyl)-6,7-dihydro-5H-pyrrolo[1,2-a]imidazol-7-ol;
(R)-7-(3-(2-(5H-Pyrrolo[2,3-b]pyrazin-7-yl)thiazol-4-yl)phenyl)-7H-pyrrolo[1,2-a]imidazol-7-ol;
(S)-7-(3-(2-(5H-Pyrrolo[2,3-b]pyrazin-7-yl)thiazol-4-yl)phenyl)-7H-pyrrolo[1,2-a]imidazol-7-ol;
(R)-7-(3-(4-(1H-Pyrrolo[2,3-b]pyridin-3-yl)thiazol-2-yl)phenyl)-6,7-dihydro-5H-pyrrolo[1,2-a]imidazol-7-ol;
(R)-3-(3-(2-(1H-Pyrrolo[2,3-b]pyridin-3-yl)thiazol-4-yl)phenyl)-2,3-dihydrofuro[3,2-b]pyridin-3-ol;
(R)-3-(3-(2-(5H-Pyrrolo[2,3-b]pyrazin-7-yl)thiazol-4-yl)phenyl)-2,3-dihydrofuro[3,2-b]pyridin-3-ol;
(S)-7-(3-(2-(5H-Pyrrolo[2,3-b]pyrazin-7-yl-6-d)thiazol-4-yl)phenyl)-6,7-dihydro-5H-pyrrolo[1,2-a]imidazol-7-ol;
(S)-7-(3-(2-(5H-Pyrrolo[2,3-b]pyrazin-7-yl-2-d)thiazol-4-yl)phenyl)-6,7-dihydro-5H-pyrrolo[1,2-a]imidazol-7-ol;
(R)-7-(3-(2-(7H-Pyrrolo[2,3-d]pyrimidin-5-yl)thiazol-4-yl)phenyl)-6,7-dihydro-5H-pyrrolo[1,2-a]imidazol-7-ol;
(S)-7-(3-(2-(7H-Pyrrolo[2,3-d]pyrimidin-5-yl)thiazol-4-yl)phenyl)-6,7-dihydro-5H-pyrrolo[1,2-a]imidazol-7-ol;
(R)-7-(3-(4-(1H-Pyrrolo[2,3-b]pyridin-3-yl)thiazol-2-yl)phenyl)-6,7-dihydro-5H-cyclopenta[b]pyridin-7-ol;
(S)-7-(3-(4-(1H-Pyrrolo[2,3-b]pyridin-3-yl)thiazol-2-yl)phenyl)-6,7-dihydro-5H-cyclopenta[b]pyridin-7-ol;
(R)-7-(3-(2-(5-Fluoro-1H-pyrrolo[2,3-b]pyridin-3-yl)thiazol-4-yl)phenyl)-6,7-dihydro-5H-pyrrolo[1,2-a]imidazol-7-ol;
(R)-7-(3-(2-(5-(Trifluoromethyl)-1H-pyrrolo[2,3-b]pyridin-3-yl)thiazol-4-yl)phenyl)-6,7-dihydro-5H-pyrrolo[1,2-a]imidazol-7-ol;
(R)-3-(4-(3-(7-Hydroxy-6,7-dihydro-5H-pyrrolo[1,2-a]imidazol-7-yl)phenyl)thiazol-2-yl)-1H-pyrrolo[2,3-b]pyridine-5-carbonitrile;
(R)-7-(3-(2-(5-Chloro-1H-pyrrolo[2,3-b]pyridin-3-yl)thiazol-4-yl)phenyl)-6,7-dihydro-5H-pyrrolo[1,2-a]imidazol-7-ol;
(R)-7-(3-(2-(5-(Methylsulfonyl)-1H-pyrrolo[2,3-b]pyridin-3-yl)thiazol-4-yl)phenyl)-6,7-dihydro-5H-pyrrolo[1,2-a]imidazol-7-ol;
(R)-7-(3-(2-(4-Fluoro-1H-pyrrolo[2,3-b]pyridin-3-yl)thiazol-4-yl)phenyl)-6,7-dihydro-5H-pyrrolo[1,2-a]imidazol-7-ol;
(R)-7-(3-(5-(1H-Pyrrolo[2,3-b]pyridin-3-yl)furan-3-yl)phenyl)-6,7-dihydro-5H-pyrrolo[1,2-a]imidazol-7-ol;
(S)-4-(3-(2-(1H-Pyrrolo[2,3-b]pyridin-3-yl)thiazol-4-yl)phenyl)-5,6-dihydro-4H-cyclopenta[d]thiazol-4-ol;
(R)-4-(3-(2-(1H-Pyrrolo[2,3-b]pyridin-3-yl)thiazol-4-yl)phenyl)-5,6-dihydro-4H-cyclopenta[d]thiazol-4-ol;
(R)-7-(3-(2-(1H-Pyrrolo[2,3-b]pyridin-3-yl)thiazol-4-yl)phenyl)-7H-cyclopenta[b]pyridin-7-ol;
(S)-7-(3-(2-(1H-Pyrrolo[2,3-b]pyridin-3-yl)thiazol-4-yl)phenyl)-7H-cyclopenta[b]pyridin-7-ol;
(S)-7-(3-(2-(1H-Pyrrolo[2,3-b]pyridin-3-yl)thiazol-4-yl)phenyl)-6,6-dimethyl-6,7-dihydro-5H-cyclopenta[b]pyridin-7-ol;
(R)-7-(3-(2-(1H-Pyrrolo[2,3-b]pyridin-3-yl)thiazol-4-yl)phenyl)-6,6-dimethyl-6,7-dihydro-5H-cyclopenta[b]pyridin-7-ol.
(S)-8-(3-(2-(1H-Pyrrolo[2,3-b]pyridin-3-yl)thiazol-4-yl)phenyl)-5,6,7,8-tetrahydroimidazo[1,2-a]pyridin-8-ol;
(R)-8-(3-(2-(1H-Pyrrolo[2,3-b]pyridin-3-yl)thiazol-4-yl)phenyl)-5,6,7,8-tetrahydroimidazo[1,2-a]pyridin-8-ol;
(S)-3-(3-(2-(5H-Pyrrolo[2,3-b]pyrazin-7-yl)thiazol-4-yl)phenyl)-3-hydroxy-1-methylpyrrolidin-2-one;
(R)-3-(3-(2-(5H-Pyrrolo[2,3-b]pyrazin-7-yl)thiazol-4-yl)phenyl)-3-hydroxy-1-methylpyrrolidin-2-one;
(R)-7-(3-(2-(5H-Pyrrolo[2,3-b]pyrazin-7-yl)thiazol-4-yl)phenyl)-6,7-dihydro-5H-pyrrolo[1,2-a]imidazol-7-ol;
(S)-7-(3-(2-(5H-Pyrrolo[2,3-b]pyrazin-7-yl)thiazol-4-yl)phenyl)-6,7-dihydro-5H-pyrrolo[1,2-a]imidazol-7-ol;
(S)-3-(3-(2-(5H-Pyrrolo[2,3-b]pyrazin-7-yl)thiazol-4-yl)phenyl)-3-hydroxy-1-(2,2,2-trifluoroethyl)pyrrolidin-2-one;
(R)-3-(3-(2-(5H-Pyrrolo[2,3-b]pyrazin-7-yl)thiazol-4-yl)phenyl)-3-hydroxy-1-(2,2,2-trifluoroethyl)pyrrolidin-2-one;
(S)-3-(3-(2-(5H-Pyrrolo[2,3-b]pyrazin-7-yl)thiazol-4-yl)phenyl)-3-hydroxy-1-methylazetidin-2-one;
(R)-3-(3-(2-(5H-Pyrrolo[2,3-b]pyrazin-7-yl)thiazol-4-yl)phenyl)-3-hydroxy-1-methylazetidin-2-one;
(R)-3-(3-(2-(1H-Pyrrolo[2,3-b]pyridin-3-yl)thiazol-4-yl)phenyl)-3-hydroxy-1-methylpyrrolidin-2-one;
(S)-3-(3-(2-(1H-Pyrrolo[2,3-b]pyridin-3-yl)thiazol-4-yl)phenyl)-3-hydroxy-1-methylpyrrolidin-2-one;
(R)-3-Hydroxy-3-(3-(2-(5-methoxy-1H-pyrrolo[2,3-b]pyridin-3-yl)thiazol-4-yl)phenyl)-1-methylpyrrolidin-2-one;
(R)-3-Hydroxy-3-(3-(5-(hydroxymethyl)-2-(1H-pyrrolo[2,3-b]pyridin-3-yl)thiazol-4-yl)phenyl)-1-methylpyrrolidin-2-one;
(R)-3-(4-(3-(3-Hydroxy-1-methyl-2-oxopyrrolidin-3-yl)phenyl)thiazol-2-yl)-1H-pyrrolo[2,3-b]pyridine-5-carboxylic acid;
(S)-3-(3-(2-(7H-Pyrrolo[2,3-d]pyrimidin-5-yl)thiazol-4-yl)phenyl)-3-hydroxy-1-methylpyrrolidin-2-one;
(R)-3-(3-(2-(7H-Pyrrolo[2,3-d]pyrimidin-5-yl)thiazol-4-yl)phenyl)-3-hydroxy-1-methylpyrrolidin-2-one;
(R)-3-(3-(2-(5H-Pyrrolo[2,3-b]pyrazin-7-yl)thiazol-4-yl)phenyl)-3-hydroxy-1-methylpiperidin-2-one;
(S)-3-(3-(2-(5H-Pyrrolo[2,3-b]pyrazin-7-yl)thiazol-4-yl)phenyl)-3-hydroxy-1-methylpiperidin-2-one;
(R)-3-(3-(4-(2-Fluoro-1H-pyrrolo[2,3-b]pyridin-3-yl)thiazol-2-yl)phenyl)-3-hydroxy-1-methylpyrrolidin-2-one;
(R)-3-(3-(2-(2-Fluoro-1H-pyrrolo[2,3-b]pyridin-3-yl)thiazol-4-yl)phenyl)-3-hydroxy-1-methylpyrrolidin-2-one;
(S)-4-(3-(4-(1H-Pyrrolo[2,3-b]pyridin-3-yl)thiazol-2-yl)phenyl)-5,6-dihydro-4H-cyclopenta[d]thiazol-4-ol;
(R)-4-(3-(4-(1H-Pyrrolo[2,3-b]pyridin-3-yl)thiazol-2-yl)phenyl)-5,6-dihydro-4H-cyclopenta[d]thiazol-4-ol;
(R)-1-(3-(2-(1H-Pyrrolo[2,3-b]pyridin-3-yl)thiazol-4-yl)phenyl)-1-(pyridin-2-yl)ethan-1-ol;
(S)-1-(3-(2-(1H-Pyrrolo[2,3-b]pyridin-3-yl)thiazol-4-yl)phenyl)-1-(pyridin-2-yl)ethan-1-ol;
(R)-7-(3-(2-(5H-Pyrrolo[2,3-b]pyrazin-7-yl-6-d)thiazol-4-yl)phenyl)-6,7-dihydro-5H-pyrrolo[1,2-a]imidazol-7-ol; and
(R)-7-(3-(2-(5H-Pyrrolo[2,3-b]pyrazin-7-yl-2-d)thiazol-4-yl)phenyl)-6,7-dihydro-5H-pyrrolo[1,2-a]imidazol-7-ol.

Embodiments of the invention relate to compounds, pharmaceutical compositions containing them, methods of making and purifying them, methods of using them as NIK inhibitors and methods for using them in the treatment of disease states, disorders, and conditions mediated by NIK.

Additional embodiments of the invention are methods of treating a subject suffering from or diagnosed with a disease, disorder, or medical condition mediated by NIK using compounds described herein.

Additional embodiments, features, and advantages of the invention will be apparent from the following detailed description and through practice of the invention.

### DETAILED DESCRIPTION

NF-κB-inducing kinase (referred to as NIK, also known as MAP3K14) is a regulator and driver of the non-canonical NIK cascade, and thus represents an attractive target for therapeutic intervention. Embodiments described herein relate to compounds that inhibit NIK and pharmaceutical compositions comprising such compounds. Compounds described herein and pharmaceutical compositions thereof are useful for preventing or treating diseases such as cancer (such as B-cell malignancies including leukemias, lymphomas and myeloma), inflammatory disorders, autoimmune disorders, immunodermatologic disorders such as palmoplantar pustulosis and hidradenitis suppurativa, and metabolic disorders such as obesity and diabetes.

NIK-dependent transcriptional activation is a tightly controlled signaling pathway, through sequential events including phosphorylation and protein degradation. In a NIK activation pathway, known as a non-canonical pathway, activation is accomplished by phosphorylating the catalytic complex subunit IKKα, leading to the partial proteolysis of the gene product p100, liberating DNA-binding protein p52 which then heterodimerizes with another DNA-binding protein RelB, translocates to the nucleus and mediates gene expression. The non-canonical pathway is activated by ligands such as CD40 ligands, B-cell activating factor (BAFF), lymphotoxin β receptor ligands, TNF-related weak inducer of apoptosis (TWEAK) cytokine, and receptor activator of nuclear factor kappa-B ligand (RANKL), also known as tumor necrosis factor ligand superfamily member 11 (TNFSF11). NIK has been shown to be required for activation of the pathway by these ligands (S.-C. Sun, Nat Rev Immunol. 2017, 17(9), 545-558). Because of its role, NIK expression is tightly regulated. Under normal non-stimulated conditions NIK protein levels are very low. This is due to its interaction with baculoviral-IAP-repeat-containing-3 (BIRC3, also known as CIAP2) and a range of TNF receptor associated factors (TRAF2 and TRAF3), which are ubiquitin ligases and result in degradation of NIK. It is believed that when the non-canonical pathway is stimulated by ligands under pathological/abnormal conditions, the activated receptors now compete for TRAFs, dissociating the TRAF-BIRC3-NIK complexes and thereby increasing the levels of NIK (For a more detailed analysis of this background, see e.g., S.-C. Sun (cited above) and Thu and Richmond, Cytokine Growth F. R. 2010, 21, 213-226). NIK plays a role propitiating immune response disorders, cell proliferation disorders, adhesion, apoptosis, and carcinogenesis, so a NIK level increase is undesirable, and one way to mitigate or eliminate the adverse effect associated with such increase is NIK inhibition.

BAFF/BAFF-R is a clinically validated therapeutic target whose inhibition is deemed beneficial for systemic lupus erythematosus (SLE) treatment. Belimumab (anti-BAFF antibody) has been approved to treat serum positive SLE patients (S. V. Navarra, et al., The Lancet, 2011; 377(9767):721-31). CD40L/CD40 pathway plays a key role in T-dependent B cell activation, dendritic cell maturation and tissue inflammation/immunity (R. Elgueta, et al., Immunol. Rev. 2009; 229(1):152-72). Anti-CD40L antibody has demonstrated promising efficacy in phase 2 clinical studies in SLE patients (P. I. Sidiropoulos and D. T. Boumpas, Lupus 2004 May; 13(5):391-7). Mice lacking NIK (R. Shinkura, et al., Nature Genetics 1999; 22(1):74-7; H. D. Brightbill, et al., J Immunol. 2015; 195(3):953-64) or conditional knockout of NIK (H. D. Brightbill, et al., J Immunol. 2015; 195(3):953-64) or human patients carrying NIK gene mutations (K. L. Willmann, et al., Nature Comm. 2014; 5:5360) showed deficiency in NIK non-canonical activation pathways such as BAFF and CD40L pathway, reduced B lymphocytes in peripheral blood, and lymphoid organs and lower T cell dependent antibody responses supporting NIK as a therapeutic target for SLE.

NIK has been characterized as being “important in the immune and bone-destructive components of inflammatory arthritis and represents a possible therapeutic target for these diseases.” K. Aya, et al. (J. Clin. Invest. 2005, 115, 1848-1854). Mice lacking functional NIK have no peripheral lymph nodes, defective B and T cells, and impaired receptor activator of NIK ligand-stimulated osteoclastogenesis. K. Aya, et al. (J. Clin. Invest. 2005, 115, 1848-1854) investigated the role of NIK in murine models of inflammatory arthritis using NIK−/− mice. The serum transfer arthritis model was initiated by preformed antibodies and required only intact neutrophil and complement systems in recipients. While NIK−/− mice had inflammation equivalent to that of NIK+/+ controls, Ada, et al., (cited above) showed significantly less periarticular osteoclastogenesis and less bone erosion. In contrast, NIK−/− mice were completely resistant to antigen-induced arthritis (AIA), which requires intact antigen presentation and lymphocyte function but not lymph nodes. Additionally, transfer of NIK+/+ splenocytes or T cells to Rag2−/− mice conferred susceptibility to AIA, while transfer of NIK−/− cells did not. NIK−/− mice were also resistant to a genetic, spontaneous form of arthritis, generated in mice expressing both the KRN T cell receptor and H-2g7. Transgenic mice were used with OC-lineage expression of NIK lacking its TRAF3 binding domain (NT3), to demonstrate that constitutive activation of NIK drives enhanced osteoclastogenesis and bone resorption, both in basal conditions and in response to inflammatory stimuli. See Aya, et al., cited above. Furthermore, constitutive activation of NIK drives enhanced osteoclastogenesis and bone resorption, both in basal conditions and in response to inflammatory stimuli. (C. Yang, et al., PLoS ONE 2010, 5(11): e15383, doi:10.1371/journal.pone.0015383).

In addition, manipulating levels of NIK in T cells can also be important for therapeutic use. Decreasing NIK activity in T cells might significantly ameliorate autoimmune responses and alloresponses, like GvHD (Graft-Versus-Host Disease) and transplant rejection, without crippling the immune system as severely as do inhibitors of another NIK activation pathway referred to as canonical pathway (S. E. Murray, et al., “NF-κB-inducing kinase plays an essential T cell-intrinsic role in graft-versus-host disease and lethal autoimmunity in mice” J. Clin. Invest. 2011; 121(12): 4775-86) (providing data that is characterized as “suggest[ing] that [NIK] tight regulation is critical for avoiding autoimmunity.”). (Canonical NIK activation pathway relies on inducible degradation of IκB kinases, particularly IκBα, leading to nuclear translocation of various NF-κB complexes, predominantly the p50/RelA dimer. The degradation of IκBα is mediated through its phosphorylation by the IκB kinase (IKK), a trimeric complex composed of two catalytic subunits, IKKα and IKKβ, and a regulatory subunit, IKKγ (also named NF-κB essential modulator or NEMO). In a non-canonical NIK activation pathway, the RelB/p52 NF-κB complex is activated using a mechanism that relies on the inducible processing of p100 instead of degradation of IκBα. See, e.g., S.-C. Sun, Cell Res. 2011 January; 21(1): 71-85).

NIK is also a therapeutic target for other BAFF, CD40L or lymphotoxin β receptor ligands driven autoimmune disorders such as Sjogren's syndrome (J. Groom, et al., J. Clin. Invest. 2002; 109(1):59-68); proliferative lupus glomerulonephritis (D. T. Boumpas, et al., Arthritis & Rheumatism 2003; 48(3):719-27): multiple sclerosis (J. Tan, et al., J. Neuroimmunol, 1999; 97(1-2):77-85), J. Krumbholz, et al., J. Exp. Med. 2005; 201(2):195-200); and pemphigus vulgaris (Z. Liu, et al., J. Invest. Dermatol. 2006; 126(1):11-3).

Blocking the NF-κB signaling pathway in cancer cells can cause cells to stop proliferating, to die and to become more sensitive to the action of other anti-cancer therapies. A role for NIK has been shown in the pathogenesis of both hematological malignancies and solid tumors.

The NF-κB pathway is dysregulated in multiple myeloma due to a range of diverse genetic abnormalities that lead to the engagement of the canonical and non-canonical pathways. Myeloma patient samples frequently have increased levels of NIK activity. This can be due to chromosomal amplification, translocations (that result in NIK proteins that have lost TRAF binding domains), mutations (in the TRAF binding domain of NIK) or TRAF loss of function mutations. Researchers have shown that myeloma cell lines can be dependent on NIK for proliferation; in these cell lines if NIK activity is reduced by either shRNA or compound inhibition, this leads to a failure in NF-κB signaling and the induction of cell death.

In a similar manner, mutations in TRAF and increased levels of NIK have also been seen in samples from Hodgkin lymphoma (HL) patients. Once again proliferation of cell lines derived from HL patients is susceptible to inhibition of NIK function by both shRNA and compounds.

NIK levels are also enhanced in adult T cell leukemia (ATL) cells and targeting NIK with shRNA reduced ATL growth in vivo. API2-MALT1 fusion oncoprotein created by the recurrent translocation t(11;18)(q21;q21) in mucosa-associated lymphoid tissue (MALT) lymphoma induces proteolytic cleavage of NF-κB-inducing kinase (NIK) at arginine 325. NIK cleavage generates a C-terminal NIK fragment that retains kinase activity and is resistant to proteasomal degradation (due to loss of TRAF binding region). The presence of this truncated NIK leads to constitutive non-canonical NF-κB signaling, enhanced B cell adhesion, and apoptosis resistance. Thus NIK inhibitors could represent a new treatment approach for refractoryt(11;18)-positive MALT lymphoma.

NIK aberrantly accumulates in diffuse large B-cell lymphoma (DLBCL) cells due to constitutive activation of B-cell activation factor (BAFF) through interaction with autochthonous B-lymphocyte stimulator (BLyS) ligand. NIK accumulation in human DLBCL cell lines and patient tumor samples suggested that constitutive NIK kinase activation is likely to be a key signaling mechanism involved in abnormal lymphoma tumor cell proliferation. Growth assays showed that using shRNA to inhibit NIK kinase protein expression in GCB- and ABC-like DLBCL cells decreased lymphoma cell growth in vitro, implicating NIK-induced NF-κB pathway activation as having a significant role in DLBCL proliferation.

Loss-of-function mutations in TRAF3 have been characterized in human and canine DLBCL. Recently, similar mutations in the non-cannonical NFkB signaling pathway (TRAF2, TRAF3, NIK, BIRC3) were found in ibrutinib-refractory mantle cell lymphoma cell lines.

The role of NIK in tumor cell proliferation is not restricted to hematological cells, there are reports that NIK protein levels are stabilized in some pancreatic cancer cell lines and as seen in blood cells proliferation of these pancreatic cancer lines are susceptible to NIK siRNA treatment. Constitutive activation of NF-κB, is preferentially involved in the proliferation of basal-like subtype breast cancer cell lines, including elevated NIK protein levels in specific lines. In melanoma tumors, tissue microarray analysis of NIK expression revealed that there was a statistically significant elevation in NIK expression when compared with benign tissue. Moreover, shRNA techniques were used to knock-down NIK, the resultant NIK-depleted melanoma cell lines exhibited decreased proliferation, increased apoptosis, delayed cell cycle progression and reduced tumor growth in a mouse xenograft model. NFκKB is often constitutively activated in non-small cell lung cancer tissue specimens and cell lines. Depletion of NIK by RNAi induced apoptosis and affected efficiency of anchorage-independent NSCLC cell growth.

As used herein, the terms “including”, “containing” and “comprising” are used in their open, non-limiting sense.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The term “C_{(a-b)}” or “Cₐ-C_{b}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₍₁₋₄₎ denotes a radical containing 1, 2, 3 or 4 carbon atoms.

The term “halo” represents chloro, fluoro, bromo, or iodo.

Unless qualified specifically in particular instances of use, the term “alkyl” refers to a straight- or branched-chain alkyl group having from 1 to 8 carbon atoms in the chain. Examples of alkyl groups include methyl (Me), ethyl (Et), n-propyl, iso-propyl, butyl, iso-butyl, sec-butyl, tert-butyl (tBu), pentyl, iso-pentyl, tert-pentyl, hexyl, iso-hexyl, and groups that in light of the ordinary skill in the art and the teachings provided herein would be considered equivalent to any one of the foregoing examples. “C₁-C₄alkyl” refers to straight- or branched-chain alkyl group having from 1 to 4 carbon atoms in the chain.

The term “haloalkyl” refers to a straight- or branched-chain alkyl group having from 1 to 6 carbon atoms in the chain optionally substituting one or more H with halo. The term “C₁-C₄ haloalkyl” as used here refers to a straight- or branched-chain alkyl group having from 1 to 4 carbon atoms in the chain, optionally substituting one or more H with halo. Examples of “haloalkyl” groups include trifluoromethyl (CF₃), difluoromethyl (CF₂H), monofluoromethyl (CH₂F), pentafluoroethyl (CF₂CF₃), tetrafluoroethyl (CHFCF₃), monofluoroethyl (CH₂CH₂F), trifluoroethyl (CH₂CF₃), tetrafluorotrifluoromethylethyl (CF(CF₃)₂), and groups that in light of the ordinary skill in the art and the teachings provided herein would be considered equivalent to any one of the foregoing examples.

The term “heterocycle” or “heterocyclyl” refers to a saturated or partially unsaturated ring system having from 3 to 11 ring atoms, wherein the ring system can have a single ring or multiple rings in a spirocyclic or bicyclic form, wherein the ring system has at least one atom other than carbon in the ring, and wherein the atom is selected from the group consisting of oxygen, nitrogen and sulfur. Exemplary heterocycles include, but are not limited to oxetanyl, aziridinyl, azetidinyl, pyrrolidinyl, piperidinyl, piperazinyl, morpholinyl, tetrahydropyranyl, tetrahydrofuranyl, thiomorpholinyl, 6,7-dihydro-5H-cyclopenta[b]pyridinyl, 6,7-dihydro-5H-pyrrolo[1,2-a]imidazolyl, 2,3-dihydrofuro[3,2-b]pyridinyl, 7H-cyclopenta[b]pyridinyl, 7H-pyrrolo[1,2-a]imidazolyl, 5,6-dihydro-4H-cyclopenta[d]thiazolyl, 5,6,7,8-tetrahydroimidazo[1,2-a]pyridinyl, 6,7-dihydro-5H-pyrrolo[1,2-a]imidazolyl, piperidin-2-one-yl, azetidin-2-one-yl, 5,6-dihydro-4H-cyclopenta[d]isoxazolyl, 1,3-dihydro-2H-pyrrolo[3,2-b]pyridin-2-one-yl, and pyrrolidin-2-one-yl.

The term “heteroaryl” refers to a single aromatic ring that has at least one atom other than carbon in the ring, wherein the atom is selected from the group consisting of oxygen, nitrogen and sulfur. The term “heteroaryl” includes single aromatic rings of from 1 to 6 carbon atoms and 1 to 4 heteroatoms selected from the group consisting of oxygen, nitrogen and sulfur. Exemplary heteroaryl ring systems include but are not limited to pyridinyl, pyrimidinyl, pyrazinyl, pyridazinyl, pyrimidinyl, pyrazolyl, oxazolyl, oxadiazolyl, isoxazolyl, triazolyl, imidazolyl, tetrazolyl, thienyl, thiazolyl, isothiazolyl, thiadiazolyl, or furanyl.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

Any formula given herein is intended to represent compounds having structures depicted by the given structural formula as well as certain variations or forms. In particular, compounds of any formula given herein may have asymmetric centers and therefore may exist in different enantiomeric forms. All optical isomers and stereoisomers of the compounds of the general formula, and mixtures thereof, are considered within the scope of such formula. The compounds of this invention may possess one or more asymmetric centers; such compounds can therefore be produced as individual (R)- or (S)-stereoisomers or as mixtures thereof. Thus, any formula given herein is intended to represent a racemate, one or more of its enantiomeric forms, one or more of its diastereomeric forms, and mixtures thereof, unless expressly indicated otherwise. The use of the term (R,S) in the name of the compound indicates that the compound is a racemate.

Reference to a compound herein stands for a reference to any one of: (a) the actually recited form of such compound, and (b) any of the forms of such compound in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH_{(aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number in an enriched form. Examples of isotopes that can be incorporated into compounds described herein in a form that exceeds natural abundances include isotopes of hydrogen, carbon, nitrogen, and oxygen such as ²H (or D), ³H, ¹¹C, ¹³C, ¹⁴C, ¹⁵N, ¹⁸O, and ¹⁷O, respectively. Such isotopically labeled compounds are useful in metabolic studies (for example with ¹⁴C), reaction kinetic studies (with, for example deuterium (i.e., D or ²H); or tritium (i.e., T or ³H)), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be used for PET or SPECT studies. Further, substitution with heavier isotopes such as deuterium may afford certain therapeutic advantages resulting from greater metabolic stability, for example increased local in vivo half-life or reduced dosage requirements. Isotopically labeled compounds of this invention can generally be prepared by carrying out the procedures disclosed in the schemes or in the examples and preparations described below by substituting a readily available isotopically labeled reagent for a non-isotopically labeled reagent.

A “pharmaceutically acceptable salt” is a salt of a compound, such as compounds of this invention, that is non-toxic, biologically tolerable, or otherwise biologically suitable for administration to the subject. See, generally, S. M. Berge, et al., “Pharmaceutical Salts”, J. Pharm. Sci. 66, 1-19 (1977); Handbook of Pharmaceutical Salts, Properties, Selection, and Use, Stahl and Wermuth, Eds., Wiley-VCH and VHCA, Zurich, 2002; and G. S. Paulekuhn, et al., “Pharmaceutical ingredient salt selection based on analysis of the Orange Book database”, J. Med. Chem. 50, 6665-72 (2007). Compounds of the invention may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of the invention contains at least one basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, and phosphoric acid, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

In addition to the illustrative embodiments listed above under the heading Brief Summary of the Invention, other illustrative embodiments of the invention are provided by any grouping of compounds in such list of compounds and/or the corresponding pharmaceutically acceptable salts thereof.

Additional illustrative embodiments of the invention are methods of treating a subject suffering from or diagnosed with a disease, disorder, or medical condition mediated by NIK activity, comprising administering to a subject in need of such treatment an effective amount of at least one of the compounds given above.

Additional illustrative embodiments of the invention are methods of treating a subject suffering from or diagnosed with a disease, disorder, or medical condition mediated by NIK activity, comprising administering to a subject in need of such treatment an effective amount of at least one of the compounds given above wherein the disease, disorder or medical condition is at least one of cancer, inflammatory disorders, autoimmune disorders, immunodermatologic disorders, and metabolic disorders.

Additional illustrative embodiments of the invention are methods of treating a subject suffering from or diagnosed with a disease, disorder, or medical condition mediated by NIK activity, comprising administering to a subject in need of such treatment an effective amount of at least one of the compounds given above wherein the disease, disorder or medical condition is at least one of SLE, RA, GvHD, transplant rejection, Sjogren's Syndrome, pemphigus vulgaris, palmoplantar pustulosis, hidradenitis suppurativa, obesity and diabetes.

Additional illustrative embodiments of the invention are compounds of Formula I, or a pharmaceutically acceptable salt thereof, for use in the treatment or in the prevention of a haematological malignancy or solid tumor.

In an additional illustrative embodiment, said haematological malignancy is selected from the group consisting of multiple myeloma, Hodgkin lymphoma, T-cell leukaemia, mucosa-associated lymphoid tissue lymphoma, diffuse large B-cell lymphoma and mantle cell lymphoma.

In an additional illustrative embodiment, the solid tumor is selected from the group consisting of pancreatic cancer, breast cancer, melanoma and non-small cell lung cancer.

Additional embodiments of the invention are pharmaceutical compositions each comprising an effective amount of at least one of the compounds given above or a pharmaceutically acceptable salt thereof.

Embodiments of this invention illustrated by compounds described herein and their pharmaceutically acceptable salts, whether alone or in combination, (collectively, “active agent” or “active agents”) are useful as NIK inhibitors in the methods of the invention. Such methods for modulating NIK activity comprise exposing NIK to an effective amount of at least one active agent of the invention.

In some embodiments, the NIK inhibitor is used in a subject diagnosed with or suffering from a disease, disorder, or medical condition mediated through NIK activity, such as those described herein. Symptoms or disease states are intended to be included within the scope of “diseases, disorders or medical conditions.”

Accordingly, the invention relates to methods of using the active agents described herein to treat subjects diagnosed with or suffering from a disease, disorder, or medical condition mediated through NIK. The term “treat” or “treating” as used herein is intended to refer to administration of an active agent or composition of the invention to a subject for the purpose of affecting a therapeutic or prophylactic benefit through modulation of NIK. Treating includes reversing, ameliorating, alleviating, inhibiting the progress of, lessening the severity of, reducing, or preventing a disease, disorder, or condition, or one or more symptoms of such disease, disorder or condition mediated through modulation of NIK activity. The term “subject” refers to a mammalian patient in need of such treatment, such as a human. The term “inhibitors” or “inhibitor” refers to compounds that decrease, prevent, inactivate, desensitize or down-regulate NIK expression or activity.

When referring to inhibiting the target, an “effective amount” means an amount sufficient to inhibit the activity of NIK.

In treatment methods according to the invention, an effective amount of at least one active agent according to the invention is administered to a subject suffering from or diagnosed as having such a disease, disorder, or medical condition. An “effective amount” means then an amount or dose sufficient to generally bring about the desired therapeutic or prophylactic benefit in patients in need of such treatment for the designated disease, disorder, or medical condition. For a 70-kg human, an illustrative range for a dosage amount is from about 1 to 1000 mg/day in single or multiple dosage units.

Once improvement of the patient's disease, disorder, or condition has occurred, the dose may be adjusted for preventive or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

A pharmaceutical composition of the invention comprises an effective amount of at least one active agent in accordance with the invention.

Pharmaceutically acceptable excipients commonly used in pharmaceutical compositions are substances that are non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of such excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using pharmaceutically acceptable excipients and compounding techniques known or that become available to those of ordinary skill in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. The compositions may be formulated for any one of a plurality of administration routes, such as intravenous infusion, subcutaneous injection, topical administration, or oral administration.

For oral administration, the active agents of the invention can be provided in the form of tablets, capsules, or beads, or as a solution, emulsion, or suspension. To prepare the oral compositions, the active agents may be formulated to yield a dosage of, e.g., for a 70-kg human, from about 1 to 1000 mg/day in single or multiple dosage units as an illustrative range.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Illustrative examples of liquid oral excipients include ethanol, glycerol, water, and the like. Starch, polyvinyl-pyrrolidone (PVP), sodium starch glycolate, microcrystalline cellulose, and alginic acid are examples of disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract or may be coated with an enteric coating. Additional coating that may be used include coatings that are designed to release the compound or active agent as a function of time, pH or bacterial content.

Capsules for oral administration include hard and soft gelatin or (hydroxypropyl)methyl cellulose capsules. To prepare hard gelatin capsules, active ingredient(s) may be mixed with a solid, semi-solid, or liquid diluent. Soft gelatin capsules may be prepared by mixing the active ingredient with an oil such as peanut oil or olive oil, liquid paraffin, a mixture of mono and di-glycerides of short chain fatty acids, polyethylene glycol 400, or propylene glycol. Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository, enema or foam. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier. In another mode of administering the agents of the invention may utilize a patch formulation to effect transdermal delivery.

Active agents may alternatively be administered in methods of this invention by inhalation, via the nasal or oral routes, e.g., in a spray formulation also containing a suitable carrier.

Some embodiments relate to NIK inhibitors for use for the prevention and/or control of excessive inflammatory response.

Some embodiments relate to compounds and pharmaceutically acceptable salts thereof as shown in Table 1, Table 2, Table 3 and Table 4.

Additional illustrative embodiments are compounds and pharmaceutically acceptable salts thereof as shown in Table 1.

Additional illustrative embodiments are compounds and pharmaceutically acceptable salts thereof as shown in Table 2.

Additional illustrative embodiments are compounds and pharmaceutically acceptable salts thereof as shown in Table 3.

Additional illustrative embodiments are compounds and pharmaceutically acceptable salts thereof as shown in Table 4.

TABLE 1

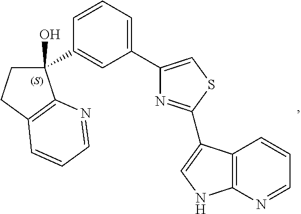

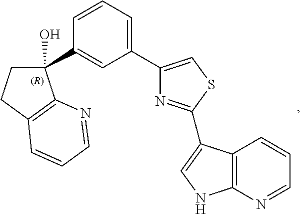

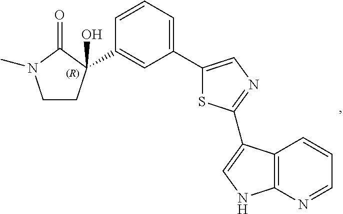

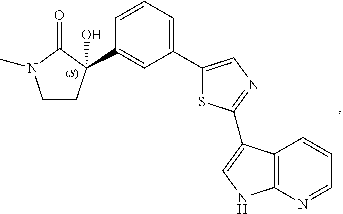

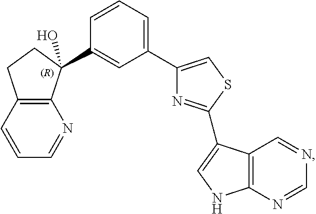

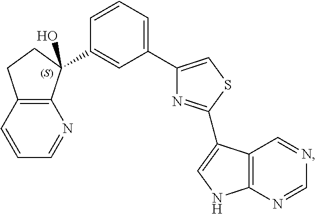

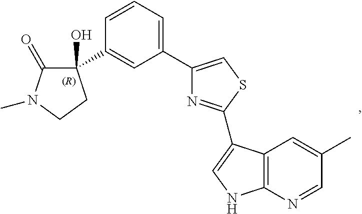

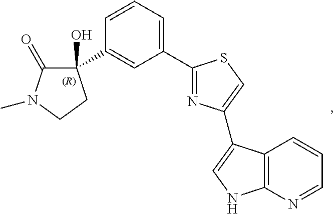

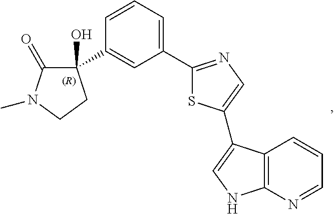

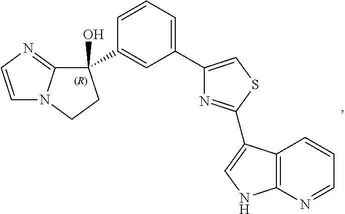

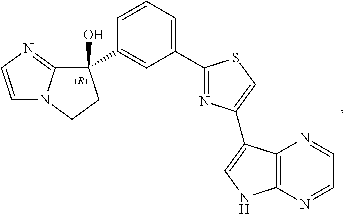

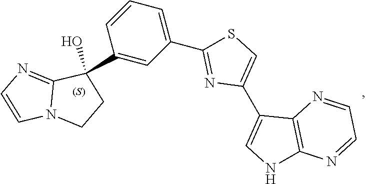

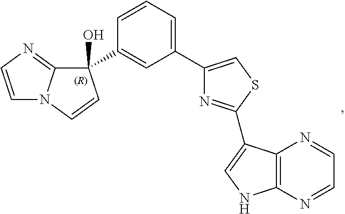

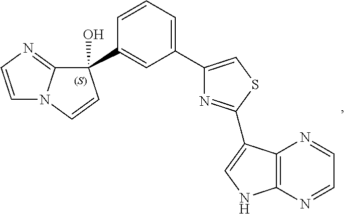

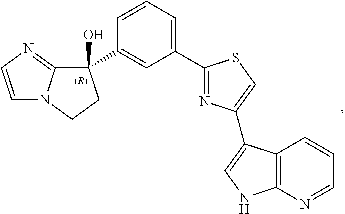

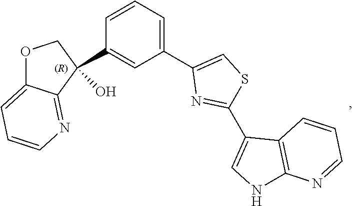

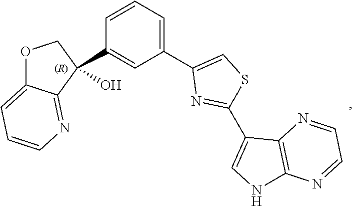

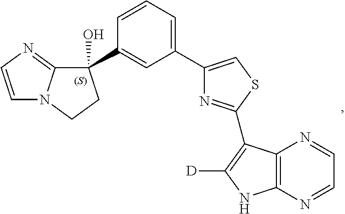

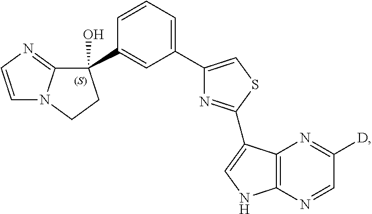

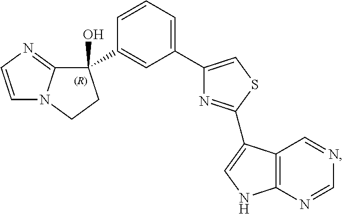

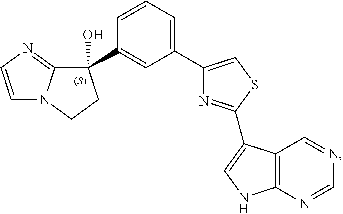

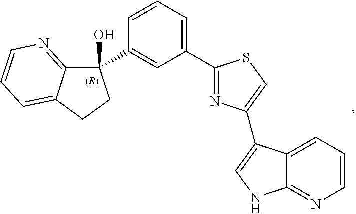

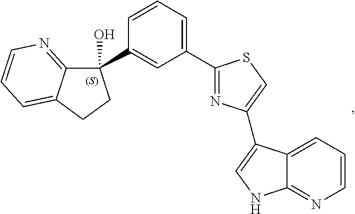

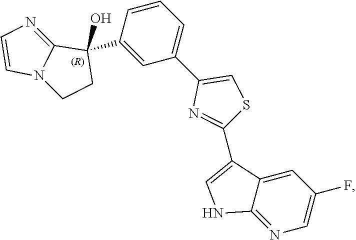

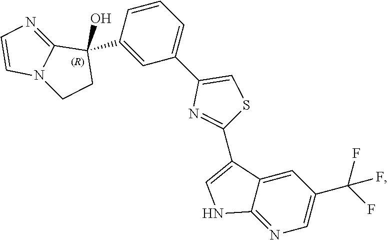

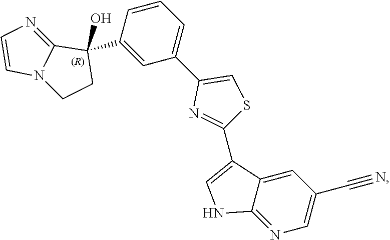

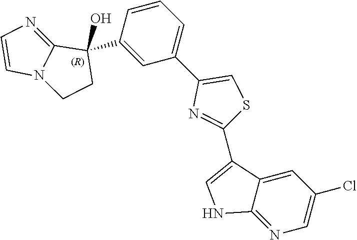

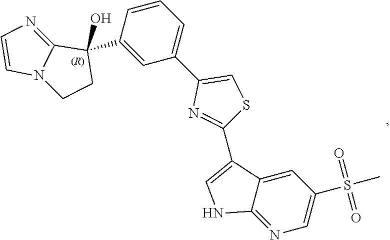

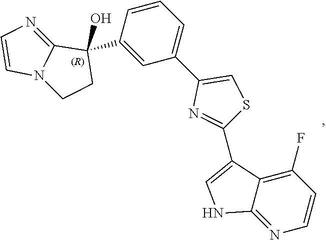

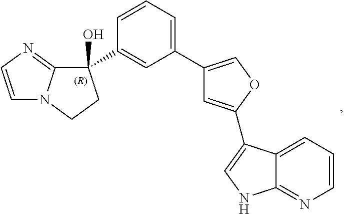

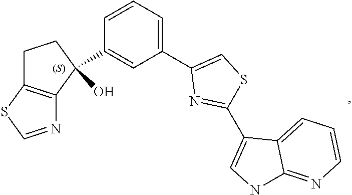

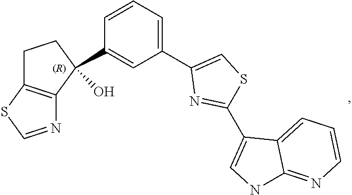

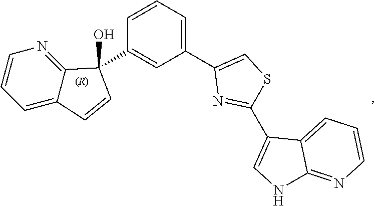

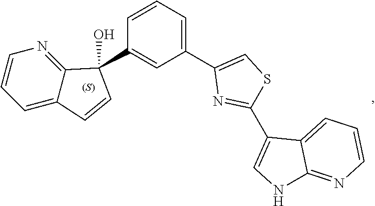

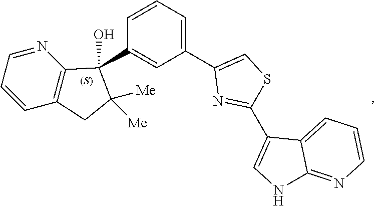

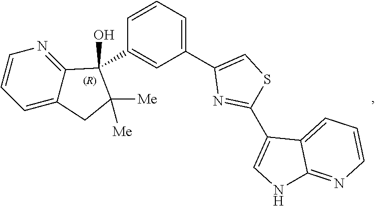

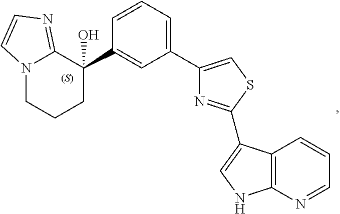

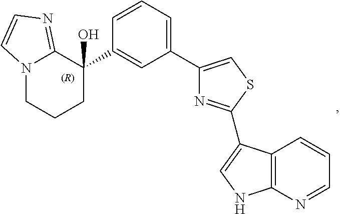

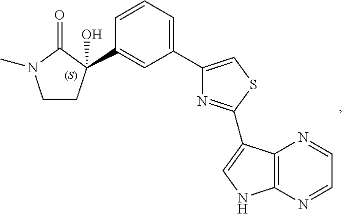

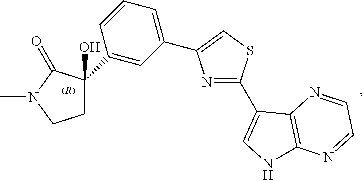

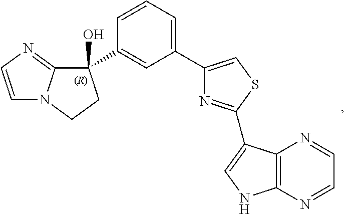

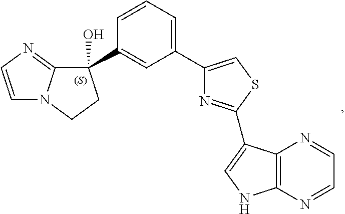

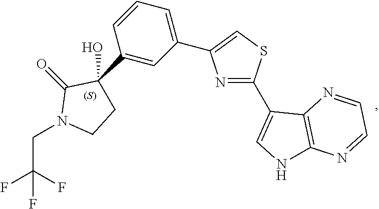

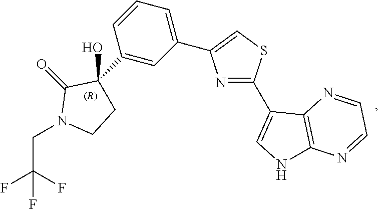

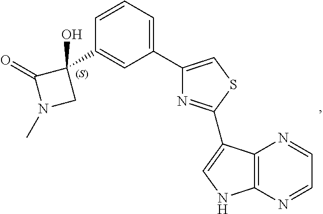

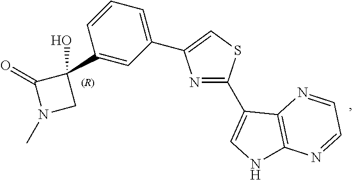

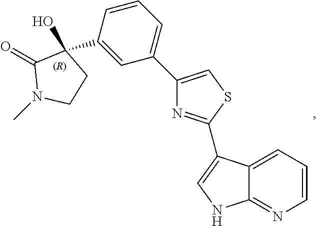

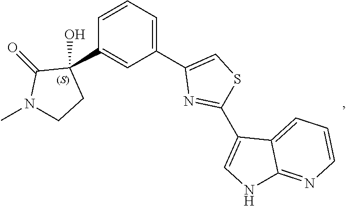

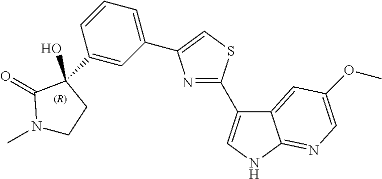

and

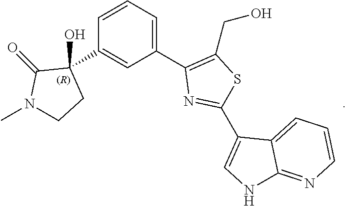

TABLE 2

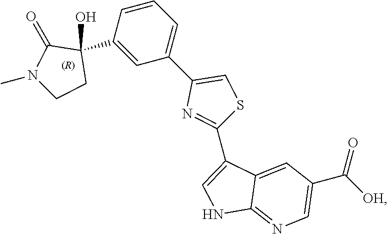

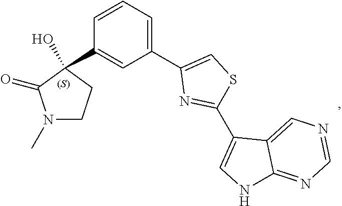

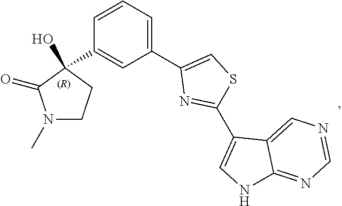

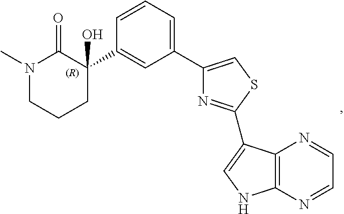

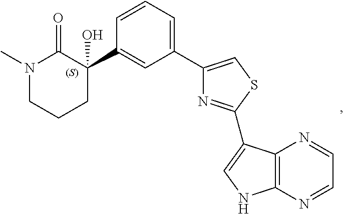

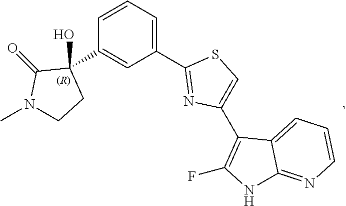

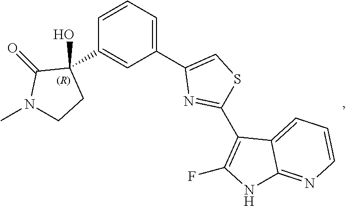

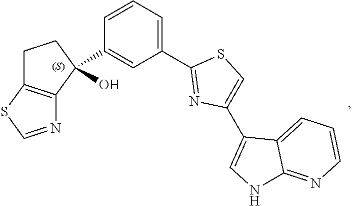

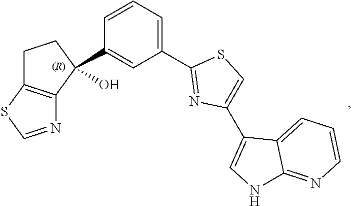

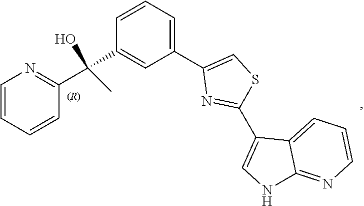

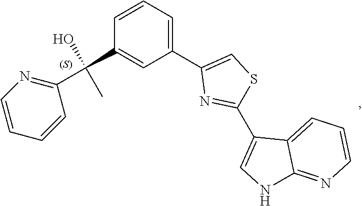

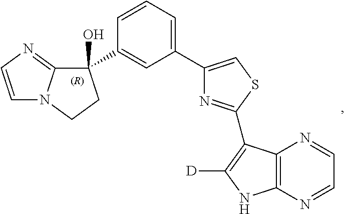

and

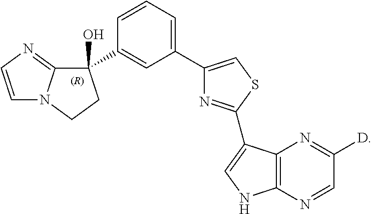

TABLE 3

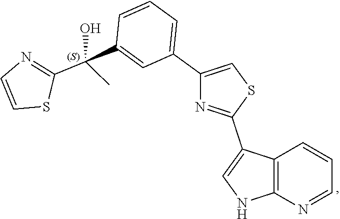

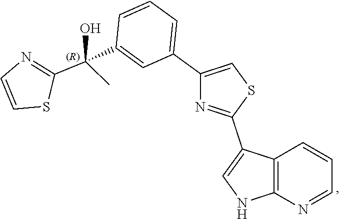

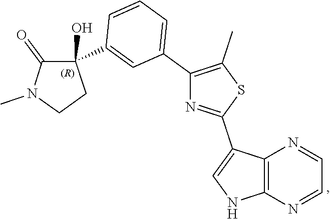

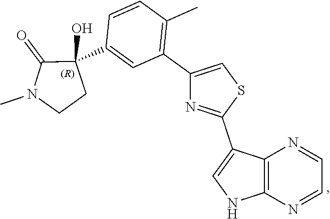

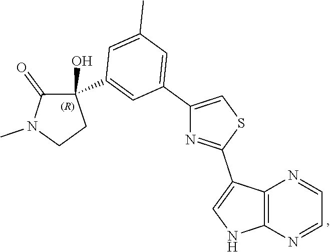

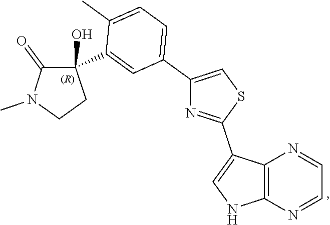

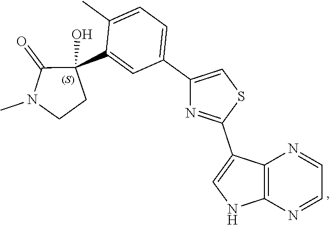

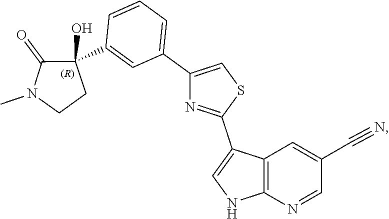

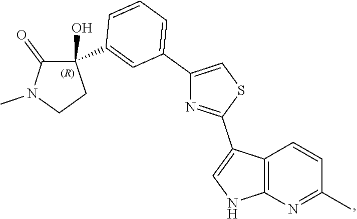

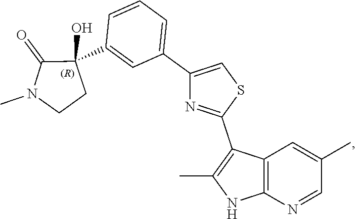

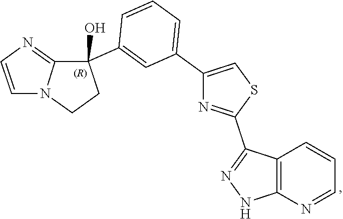

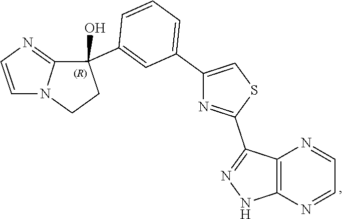

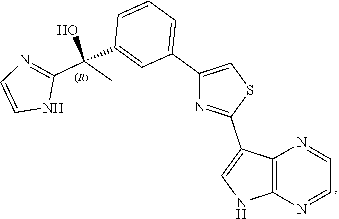

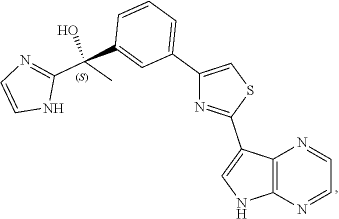

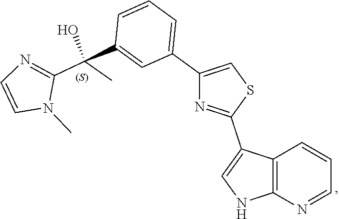

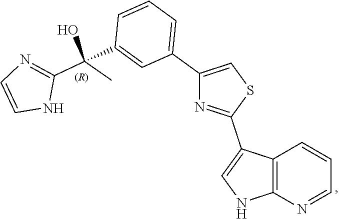

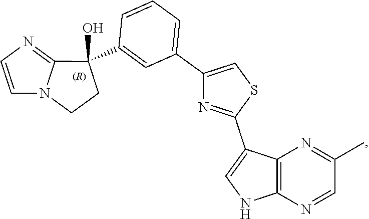

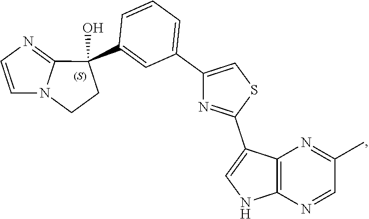

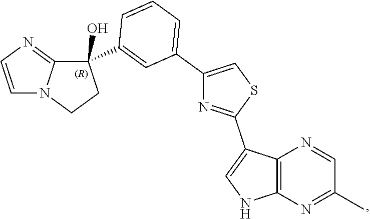

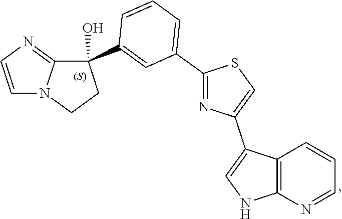

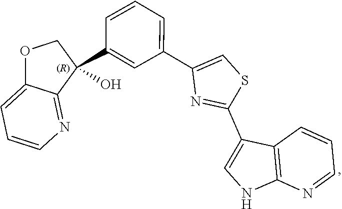

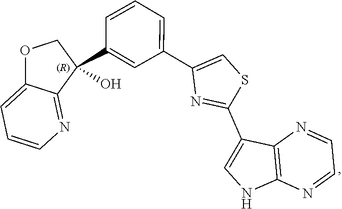

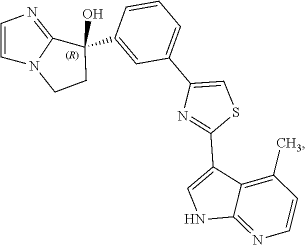

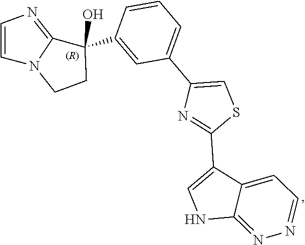

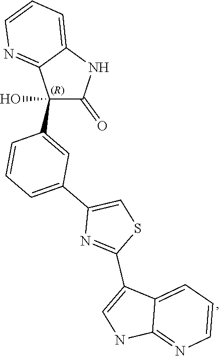

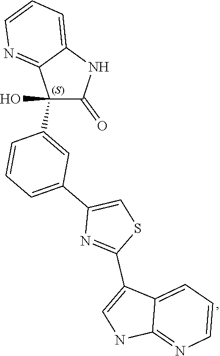

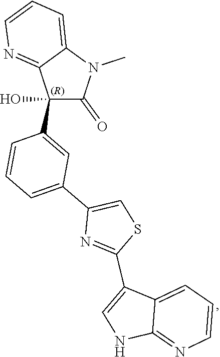

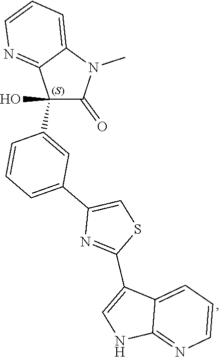

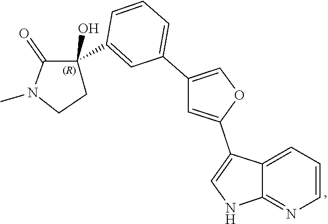

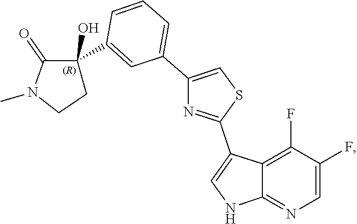

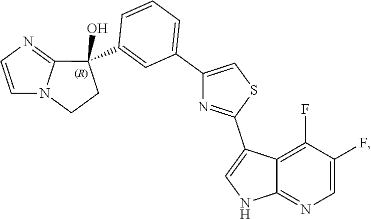

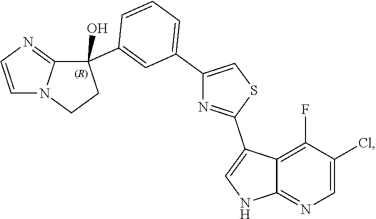

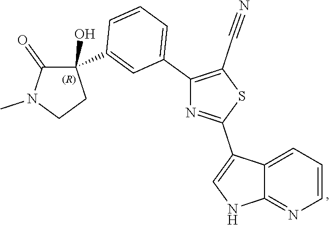

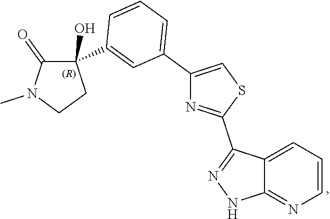

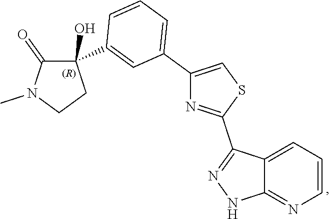

TABLE 4

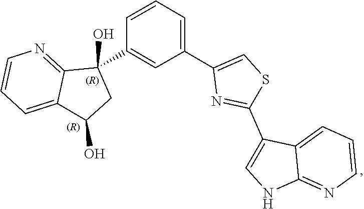

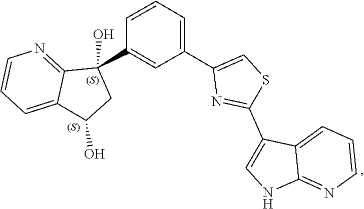

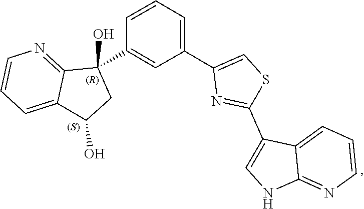

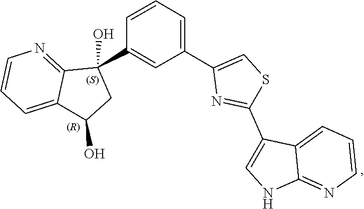

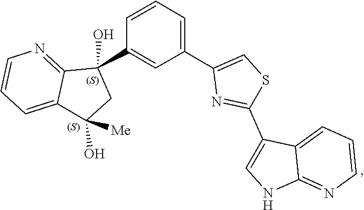

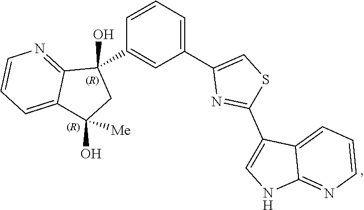

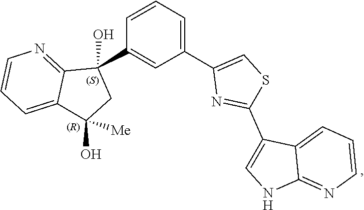

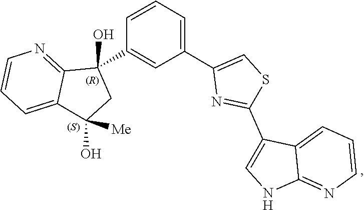

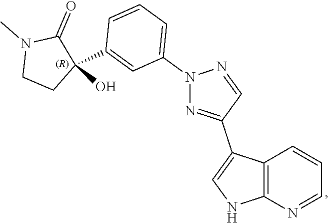

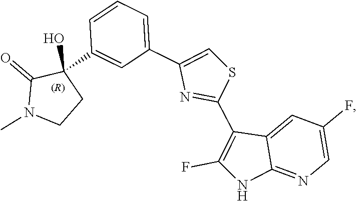

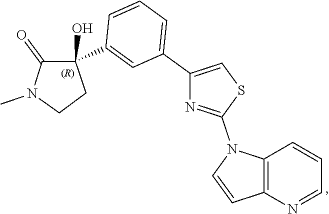

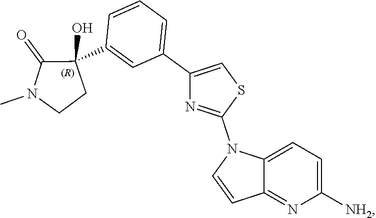

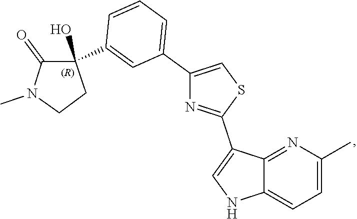

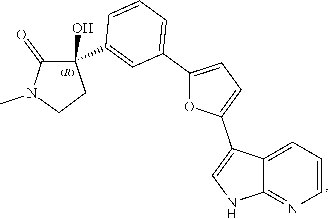

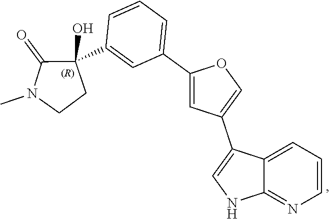

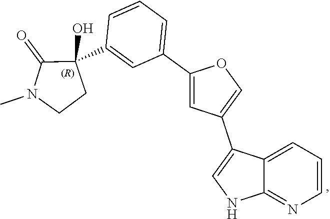

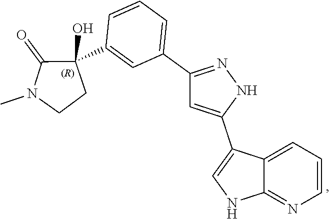

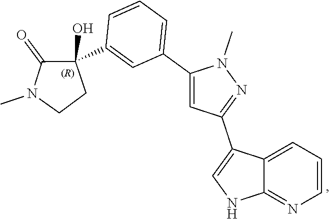

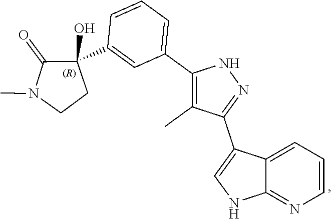

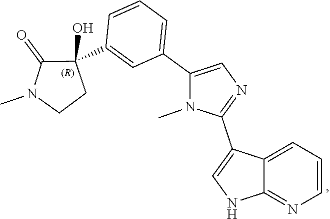

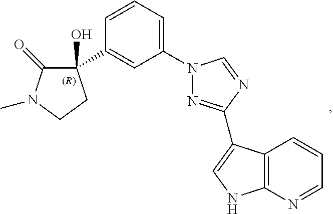

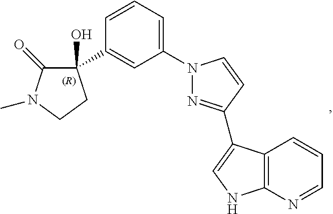

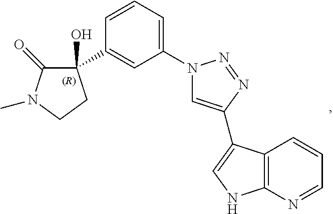

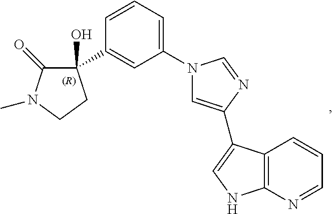

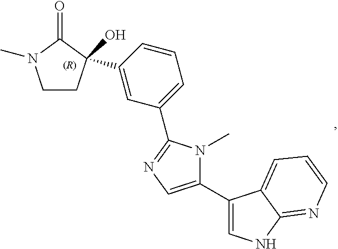

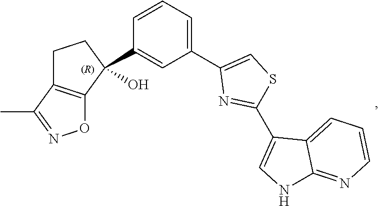

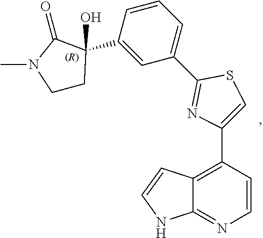

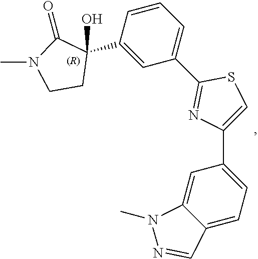

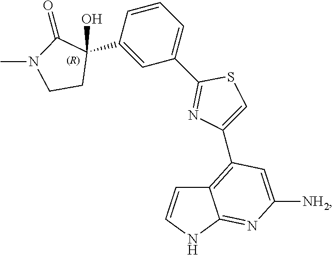

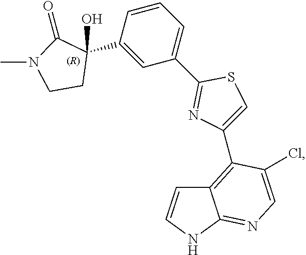

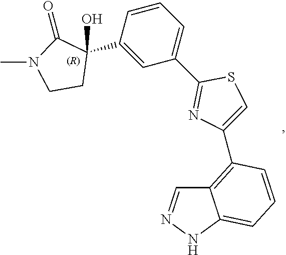

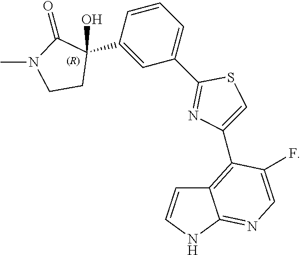

Additional illustrative embodiments are compounds of Formula I or a pharmaceutically acceptable salt thereof,

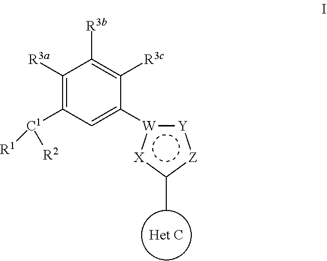

wherein
C¹ is —C(OH)—,
R¹ is C₁-C₃ alkyl;
R² is a 5 or 6 membered substituted or unsubstituted heteroaryl;
or R¹ and R² can be taken together with the carbon atom to which they are attached to form a substituted or unsubstituted heterocyclyl;
R^{3a}, R^{3b} and R^{3c} are independently H or C₁-C₃ alkyl;

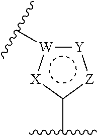

is a substituted or unsubstituted heteroaryl,
X, Y and Z are independently selected from O, S, N, N—R⁴ or C—R⁵;
W is N or C;
R⁴ is H or —C₁-C₃ alkyl; and
R⁵ is H, halo, CN, —C₁-C₃ alkyl or CH₂OH;
Het C is selected from the group consisting of

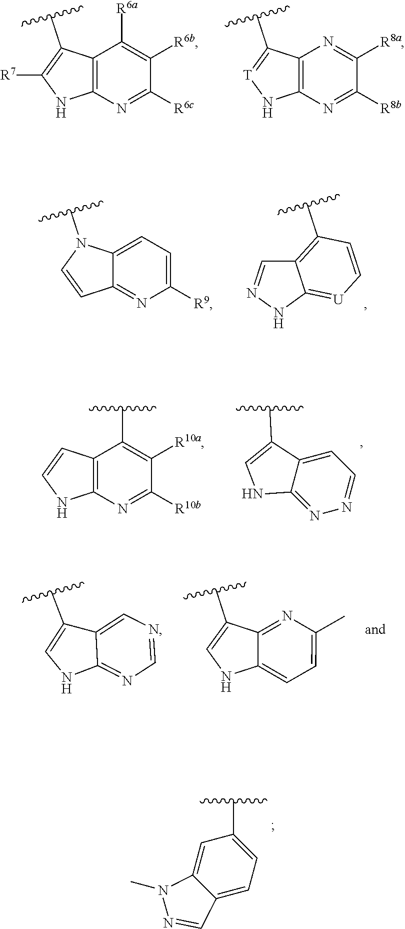

R^{6a}, R^{6b}, R^{6c} is independently H, halo, —C₁-C₃ alkyl, —C₁-C₃ haloalkyl, —CN, —CO₂H, —OC₁-C₃ alkyl, —SO₂CH₃ or —NH₂;
R⁷ is H, D, halo or —C₁-C₃ alkyl;
R^{6a}, R^{6b}, and R^{6c} is independently H, D, halo or —C₁-C₃ alkyl;
R⁹, R^{10a} and R^{10b} is independently H or NH₂;
T is N or C-Rec; and
U is N or CH.

In some embodiments, the haloalkyl is —CF₃.

In some embodiments, R^{6a}, R^{6b}, Roc is independently H, halo, —C₁-C₃ alkyl, —CF₃, CN, —CO₂H, —OC₁-C₃ alkyl, —SO₂CH₃ or —NH₂.

In some embodiments, R^{6a} is H. In some embodiments, R^{6a} is halo. In some embodiments, R^{6a} is —C₁-C₃ alkyl. In some embodiments, R^{6a} is —C₁-C₃ haloalkyl. In some embodiments, R^{6a} is —CF₃. In some embodiments, R^{6a} is —CN. In some embodiments, Rea is —CO₂H. In some embodiments, R^{6a} is —OC₁-C₃ alkyl. In some embodiments, Rea is —SO₂CH₃. In some embodiments, R^{6a} is —NH₂.

In some embodiments, R^{6b} is H. In some embodiments, R^{8b} is halo. In some embodiments, R^{6b} is —C₁-C₃ alkyl. In some embodiments, R^{6b} is —C₁-C₃ haloalkyl. In some embodiments, R^{6b} is —CF₃. In some embodiments, R^{6b} is —CN. In some embodiments, Reb is —CO₂H. In some embodiments, R^{6b} is —OC₁-C₃ alkyl. In some embodiments, Rea is —SO₂CH₃. In some embodiments, R^{6b} is —NH₂.

In some embodiments, R^{6c} is H. In some embodiments, Rho is halo. In some embodiments, R^{6c} is —C₁-C₃ alkyl. In some embodiments, R^{6c} is —C₁-C₃ haloalkyl. In some embodiments, R^{6c} is —CF₃. In some embodiments, R^{6c} is —CN. In some embodiments, R^{6c} is —CO₂H. In some embodiments, R^{6c} is —OC₁-C₃ alkyl. In some embodiments, R^{6c} is —SO₂CH₃. In some embodiments, R^{6c} is —NH₂.

In some embodiments, R⁷ is H. In some embodiments, R⁷ is D. In some embodiments, R⁷ is halo. In some embodiments, R⁷ is —C₁-C₃ alkyl.

In some embodiments, R^{8a} is H. In some embodiments, Rae is D. In some embodiments, Rae is halo. In some embodiments, Rae is —C₁-C₃ alkyl.

In some embodiments, R^{8b} is H. In some embodiments, R^{8b} is D. In some embodiments, R^{6b} is halo. In some embodiments, RN is —C₁-C₃ alkyl.

In some embodiments, R^{8c} is H. In some embodiments, R^{8c} is D. In some embodiments, R^{8c} is halo. In some embodiments, Rao is —C₁-C₃ alkyl.

In some embodiments, R⁹ is independently H. In some embodiments, R⁹ is independently NH₂.

In some embodiments, R^{10a} is independently H. In some embodiments, R^{10a} is independently NH₂.

In some embodiments, R^{10b} is independently H. In some embodiments, R^{10b} is independently NH₂.

In some embodiments, for compounds of Formula I or a pharmaceutically acceptable salt thereof, wherein R¹ is —C₁-C₃ alkyl. In some embodiments, R¹ is methyl.

In some embodiments, R² is a 5-8 membered heteroaryl containing one or more nitrogen atoms. In some embodiments, R² is pyridinyl, thiazolyl, or imidazolyl, and wherein the heteroaryl ring is unsubstituted or substituted with —C₁-C₃ alkyl.

Additional illustrative embodiments of the invention are compounds of Formula I or a pharmaceutically acceptable salt thereof, wherein R² is

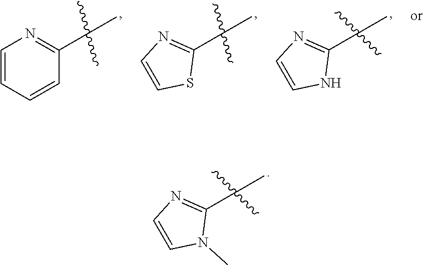

Additional illustrative embodiments of the invention are compounds of Formula I or a pharmaceutically acceptable salt thereof, wherein R¹ and R² are taken together with the carbon atom to which they are attached to form a substituted or unsubstituted heterocycle containing at least one nitrogen atom.

Additional illustrative embodiments of the invention are compounds of Formula I or a pharmaceutically acceptable salt thereof, wherein R¹ and R² are taken together with the carbon atom to which they are attached to form a heterocycle selected from the group consisting of 6,7-dihydro-5H-cyclopenta[b]pyridinyl, 6,7-dihydro-5H-pyrrolo[1,2-a]imidazolyl, 2,3-dihydrofuro[3,2-b]pyridinyl, 7H-cyclopenta[b]pyridinyl, 7H-pyrrolo[1,2-a]imidazolyl, 5,6-dihydro-4H-cyclopenta[d]thiazolyl, 5,6,7,8-tetrahydroimidazo[1,2-a]pyridinyl, 6,7-dihydro-5H-pyrrolo[1,2-a]imidazolyl, piperidin-2-one-yl, azetidin-2-one-yl, 5,6-dihydro-4H-cyclopenta[d]isoxazolyl, 1,3-dihydro-2H-pyrrolo[3,2-b]pyridin-2-one-yl, and pyrrolidin-2-one-yl.

Additional illustrative embodiments of the invention are compounds of Formula I or a pharmaceutically acceptable salt thereof, wherein R¹ and R² are taken together with —C(OH)— to which they are attached to form a heterocycle selected from the group consisting of

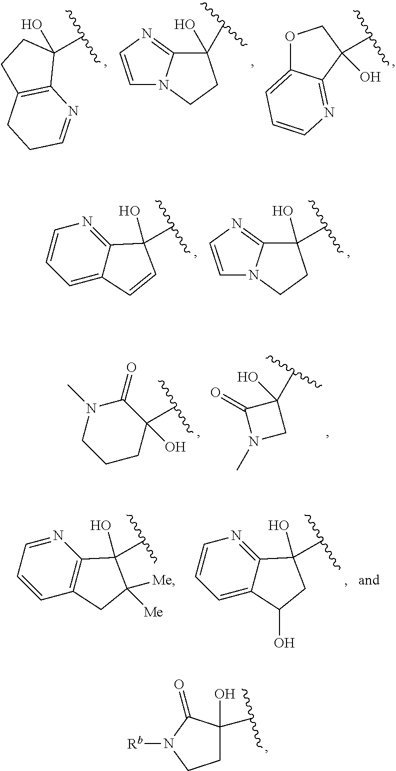

and wherein R^{a} is H, D, or Me, and R^{b} is Me or CH₂CF₃.

Additional illustrative embodiments of the invention are compounds of Formula I or a pharmaceutically acceptable salt thereof, wherein R^{3a} is CH₃ and R^{3b} and R^{3c} are H.

Additional illustrative embodiments of the invention are compounds of Formula I or a pharmaceutically acceptable salt thereof, wherein R^{3b} is CH₃ and R^{3a} and R^{3c} are H.

Additional illustrative embodiments of the invention are compounds of Formula I or a pharmaceutically acceptable salt thereof, wherein R^{3c} is CH₃ and R^{3a} and R^{3b} are H.

Additional illustrative embodiments of the invention are compounds of Formula I or a pharmaceutically acceptable salt thereof, wherein W is C.

Additional illustrative embodiments of the invention are compounds of Formula I or a pharmaceutically acceptable salt thereof, wherein X is N.

Additional illustrative embodiments of the invention are compounds of Formula I or a pharmaceutically acceptable salt thereof, wherein Y is C—R⁵.

Additional illustrative embodiments of the invention are compounds of Formula I or a pharmaceutically acceptable salt thereof, wherein

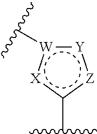

is a substituted or unsubstituted ring selected from imidazolyl, pyrazolyl, triazolyl, furanyl, thiazolyl, and oxazolyl.

Additional illustrative embodiments of the invention are compounds of Formula I or a pharmaceutically acceptable salt thereof, wherein

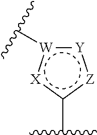

is

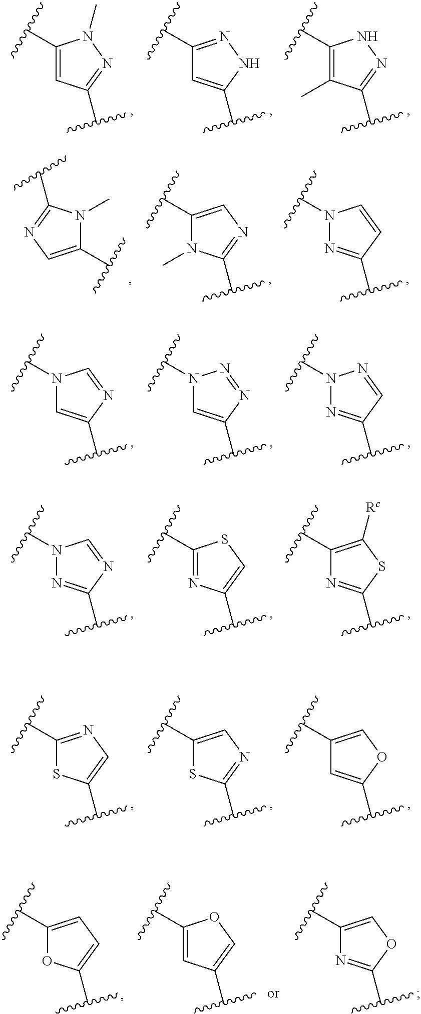

and wherein R^{c} is H, halo, CN, —C₁-C₃ alkyl or CH₂OH.

Additional illustrative embodiments of the invention are compounds of Formula I or a pharmaceutically acceptable salt thereof, wherein

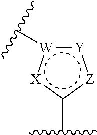

is

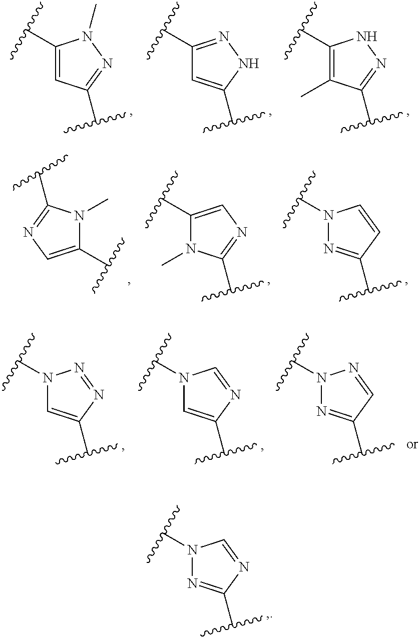

Additional illustrative embodiments of the invention are compounds of Formula I or a pharmaceutically acceptable salt thereof, wherein

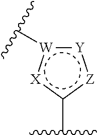

is

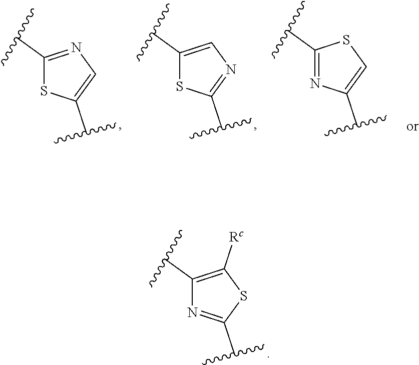

and R^{c} is H, F, CN, —C₁-C₃ alkyl or CH₂OH.

Additional illustrative embodiments of the invention are compounds of Formula I or a pharmaceutically acceptable salt thereof, wherein Het C is

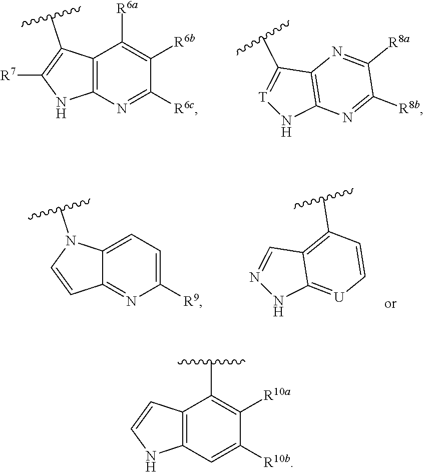

Additional illustrative embodiments of the invention are compounds of Formula I or a pharmaceutically acceptable salt thereof, wherein Het C is

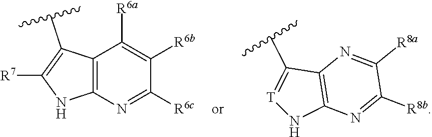

Additional illustrative embodiments of the invention are compounds of Formula I or a pharmaceutically acceptable salt thereof, wherein Het C is

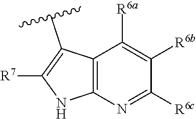

Additional illustrative embodiments of the invention are compounds of Formula I or a pharmaceutically acceptable salt thereof, wherein Het C is

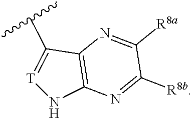

Additional illustrative embodiments of the invention are compounds of Formula I or a pharmaceutically acceptable salt thereof, wherein the absolute stereochemistry of the carbon atom of C¹ is (R).

Additional illustrative embodiments of the invention are compounds of Formula I or a pharmaceutically acceptable salt thereof, wherein the absolute stereochemistry of the carbon atom of C¹ is (S).

Additional illustrative embodiments of the invention are compounds selected from
(S)-7-(3-(2-(1H-Pyrrolo[2,3-b]pyridin-3-yl)thiazol-4-yl)phenyl)-6,7-dihydro-5H-cyclopenta[b]pyridin-7-ol;
(R)-7-(3-(2-(1H-Pyrrolo[2,3-b]pyridin-3-yl)thiazol-4-yl)phenyl)-6,7-dihydro-5H-cyclopenta[b]pyridin-7-ol;
(R)-3-(3-(2-(1H-Pyrrolo[2,3-b]pyridin-3-yl)thiazol-5-yl)phenyl)-3-hydroxy-1-methylpyrrolidin-2-one;
(R)-7-(3-(2-(7H-Pyrrolo[2,3-d]pyrimidin-5-yl)thiazol-4-yl)phenyl)-6,7-dihydro-5H-cyclopenta[b]pyridin-7-ol;
(S)-7-(3-(2-(7H-Pyrrolo[2,3-d]pyrimidin-5-yl)thiazol-4-yl)phenyl)-6,7-dihydro-5H-cyclopenta[b]pyridin-7-ol;
(R)-3-Hydroxy-1-methyl-3-(3-(2-(5-methyl-1H-pyrrolo[2,3-b]pyridin-3-yl)thiazol-4-yl)phenyl)pyrrolidin-2-one;
(R)-3-(3-(4-(1H-Pyrrolo[2,3-b]pyridin-3-yl)thiazol-2-yl)phenyl)-3-hydroxy-1-methylpyrrolidin-2-one;
(R)-7-(3-(2-(1H-Pyrrolo[2,3-b]pyridin-3-yl)thiazol-4-yl)phenyl)-6,7-dihydro-5H-pyrrolo[1,2-a]imidazol-7-ol;
(R)-7-(3-(4-(5H-Pyrrolo[2,3-b]pyrazin-7-yl)thiazol-2-yl)phenyl)-6,7-dihydro-5H-pyrrolo[1,2-a]imidazol-7-ol;
(S)-7-(3-(4-(5H-Pyrrolo[2,3-b]pyrazin-7-yl)thiazol-2-yl)phenyl)-6,7-dihydro-5H-pyrrolo[1,2-a]imidazol-7-ol;
(R)-7-(3-(2-(5H-Pyrrolo[2,3-b]pyrazin-7-yl)thiazol-4-yl)phenyl)-7H-pyrrolo[1,2-a]imidazol-7-ol;
(S)-7-(3-(2-(5H-Pyrrolo[2,3-b]pyrazin-7-yl)thiazol-4-yl)phenyl)-7H-pyrrolo[1,2-a]imidazol-7-ol;
(R)-7-(3-(4-(1H-Pyrrolo[2,3-b]pyridin-3-yl)thiazol-2-yl)phenyl)-6,7-dihydro-5H-pyrrolo[1,2-a]imidazol-7-ol;
(R)-3-(3-(2-(1H-Pyrrolo[2,3-b]pyridin-3-yl)thiazol-4-yl)phenyl)-2,3-dihydrofuro[3,2-b]pyridin-3-ol;
(R)-3-(3-(2-(5H-Pyrrolo[2,3-b]pyrazin-7-yl)thiazol-4-yl)phenyl)-2,3-dihydrofuro[3,2-b]pyridin-3-ol;
(S)-7-(3-(2-(5H-Pyrrolo[2,3-b]pyrazin-7-yl-2-d)thiazol-4-yl)phenyl)-6,7-dihydro-5H-pyrrolo[1,2-a]imidazol-7-ol;
(R)-7-(3-(2-(7H-Pyrrolo[2,3-d]pyrimidin-5-yl)thiazol-4-yl)phenyl)-6,7-dihydro-5H-pyrrolo[1,2-a]imidazol-7-ol;
(S)-7-(3-(2-(7H-Pyrrolo[2,3-d]pyrimidin-5-yl)thiazol-4-yl)phenyl)-6,7-dihydro-5H-pyrrolo[1,2-a]imidazol-7-ol;
(R)-7-(3-(4-(1H-Pyrrolo[2,3-b]pyridin-3-yl)thiazol-2-yl)phenyl)-6,7-dihydro-5H-cyclopenta[b]pyridin-7-ol;
(S)-7-(3-(4-(1H-Pyrrolo[2,3-b]pyridin-3-yl)thiazol-2-yl)phenyl)-6,7-dihydro-5H-cyclopenta[b]pyridin-7-ol;
(R)-7-(3-(2-(5-Fluoro-1H-pyrrolo[2,3-b]pyridin-3-yl)thiazol-4-yl)phenyl)-6,7-dihydro-5H-pyrrolo[1,2-a]imidazol-7-ol;
(R)-7-(3-(2-(5-(Trifluoromethyl)-1H-pyrrolo[2,3-b]pyridin-3-yl)thiazol-4-yl)phenyl)-6,7-dihydro-5H-pyrrolo[1,2-a]imidazol-7-ol;
(R)-3-(4-(3-(7-Hydroxy-6,7-dihydro-5H-pyrrolo[1,2-a]imidazol-7-yl)phenyl)thiazol-2-yl)-1H-pyrrolo[2,3-b]pyridine-5-carbonitrile;
(R)-7-(3-(2-(5-Chloro-1H-pyrrolo[2,3-b]pyridin-3-yl)thiazol-4-yl)phenyl)-6,7-dihydro-5H-pyrrolo[1,2-a]imidazol-7-ol;
(R)-7-(3-(2-(5-(Methylsulfonyl)-1H-pyrrolo[2,3-b]pyridin-3-yl)thiazol-4-yl)phenyl)-6,7-dihydro-5H-pyrrolo[1,2-a]imidazol-7-ol;
(R)-7-(3-(2-(4-Fluoro-1H-pyrrolo[2,3-b]pyridin-3-yl)thiazol-4-yl)phenyl)-6,7-dihydro-5H-pyrrolo[1,2-a]imidazol-7-ol;
(R)-7-(3-(5-(1H-Pyrrolo[2,3-b]pyridin-3-yl)furan-3-yl)phenyl)-6,7-dihydro-5H-pyrrolo[1,2-a]imidazol-7-ol;
(S)-4-(3-(2-(1H-Pyrrolo[2,3-b]pyridin-3-yl)thiazol-4-yl)phenyl)-5,6-dihydro-4H-cyclopenta[d]thiazol-4-ol;
(R)-4-(3-(2-(1H-Pyrrolo[2,3-b]pyridin-3-yl)thiazol-4-yl)phenyl)-5,6-dihydro-4H-cyclopenta[d]thiazol-4-ol;
(R)-7-(3-(2-(1H-Pyrrolo[2,3-b]pyridin-3-yl)thiazol-4-yl)phenyl)-7H-cyclopenta[b]pyridin-7-ol;
(R)-7-(3-(2-(1H-Pyrrolo[2,3-b]pyridin-3-yl)thiazol-4-yl)phenyl)-6,6-dimethyl-6,7-dihydro-5H-cyclopenta[b]pyridin-7-ol.
(S)-8-(3-(2-(1H-Pyrrolo[2,3-b]pyridin-3-yl)thiazol-4-yl)phenyl)-5,6,7,8-tetrahydroimidazo[1,2-a]pyridin-8-ol;
(R)-8-(3-(2-(1H-Pyrrolo[2,3-b]pyridin-3-yl)thiazol-4-yl)phenyl)-5,6,7,8-tetrahydroimidazo[1,2-a]pyridin-8-ol;
(R)-3-(3-(2-(5H-Pyrrolo[2,3-b]pyrazin-7-yl)thiazol-4-yl)phenyl)-3-hydroxy-1-methylpyrrolidin-2-one;
(R)-7-(3-(2-(5H-Pyrrolo[2,3-b]pyrazin-7-yl)thiazol-4-yl)phenyl)-6,7-dihydro-5H-pyrrolo[1,2-a]imidazol-7-ol;
(S)-7-(3-(2-(5H-Pyrrolo[2,3-b]pyrazin-7-yl)thiazol-4-yl)phenyl)-6,7-dihydro-5H-pyrrolo[1,2-a]imidazol-7-ol;
(R)-3-(3-(2-(5H-Pyrrolo[2,3-b]pyrazin-7-yl)thiazol-4-yl)phenyl)-3-hydroxy-1-(2,2,2-trifluoroethyl)pyrrolidin-2-one;
(R)-3-(3-(2-(5H-Pyrrolo[2,3-b]pyrazin-7-yl)thiazol-4-yl)phenyl)-3-hydroxy-1-methylazetidin-2-one;
(R)-3-(3-(2-(1H-Pyrrolo[2,3-b]pyridin-3-yl)thiazol-4-yl)phenyl)-3-hydroxy-1-methylpyrrolidin-2-one;
(S)-3-(3-(2-(1H-Pyrrolo[2,3-b]pyridin-3-yl)thiazol-4-yl)phenyl)-3-hydroxy-1-methylpyrrolidin-2-one;
(R)-3-Hydroxy-3-(3-(2-(5-methoxy-1H-pyrrolo[2,3-b]pyridin-3-yl)thiazol-4-yl)phenyl)-1-methylpyrrolidin-2-one;
(R)-3-Hydroxy-3-(3-(5-(hydroxymethyl)-2-(1H-pyrrolo[2,3-b]pyridin-3-yl)thiazol-4-yl)phenyl)-1-methylpyrrolidin-2-one;
(R)-3-(4-(3-(3-Hydroxy-1-methyl-2-oxopyrrolidin-3-yl)phenyl)thiazol-2-yl)-1H-pyrrolo[2,3-b]pyridine-5-carboxylic acid;
(S)-3-(3-(2-(7H-Pyrrolo[2,3-d]pyrimidin-5-yl)thiazol-4-yl)phenyl)-3-hydroxy-1-methylpyrrolidin-2-one;
(R)-3-(3-(2-(7H-Pyrrolo[2,3-d]pyrimidin-5-yl)thiazol-4-yl)phenyl)-3-hydroxy-1-methylpyrrolidin-2-one;
(R)-3-(3-(2-(5H-Pyrrolo[2,3-b]pyrazin-7-yl)thiazol-4-yl)phenyl)-3-hydroxy-1-methylpiperidin-2-one;
(R)-3-(3-(4-(2-Fluoro-1H-pyrrolo[2,3-b]pyridin-3-yl)thiazol-2-yl)phenyl)-3-hydroxy-1-methylpyrrolidin-2-one;
(R)-3-(3-(2-(2-Fluoro-1H-pyrrolo[2,3-b]pyridin-3-yl)thiazol-4-yl)phenyl)-3-hydroxy-1-methylpyrrolidin-2-one;
(S)-4-(3-(4-(1H-Pyrrolo[2,3-b]pyridin-3-yl)thiazol-2-yl)phenyl)-5,6-dihydro-4H-cyclopenta[d]thiazol-4-ol;
(R)-4-(3-(4-(1H-Pyrrolo[2,3-b]pyridin-3-yl)thiazol-2-yl)phenyl)-5,6-dihydro-4H-cyclopenta[d]thiazol-4-ol;
(R)-1-(3-(2-(1H-Pyrrolo[2,3-b]pyridin-3-yl)thiazol-4-yl)phenyl)-1-(pyridin-2-yl)ethan-1-ol;
(R)-7-(3-(2-(5H-Pyrrolo[2,3-b]pyrazin-7-yl-6-d)thiazol-4-yl)phenyl)-6,7-dihydro-5H-pyrrolo[1,2-a]imidazol-7-ol;
(R)-7-(3-(2-(5H-Pyrrolo[2,3-b]pyrazin-7-yl-2-d)thiazol-4-yl)phenyl)-6,7-dihydro-5H-pyrrolo[1,2-a]imidazol-7-ol and pharmaceutically acceptable salts thereof.
Additional illustrative embodiments of the invention are compounds selected from
(R)-4-(3-(2-(1H-Pyrrolo[2,3-b]pyridin-3-yl)thiazol-4-yl)phenyl)-5,6-dihydro-4H-cyclopenta[d]thiazol-4-ol;
(R)-3-(3-(2-(1H-pyrrolo[2,3-b]pyridin-3-yl)thiazol-5-yl)phenyl)-3-hydroxy-1-methylpyrrolidin-2-one;
(R)-3-(3-(2-(5H-Pyrrolo[2,3-b]pyrazin-7-yl)thiazol-4-yl)phenyl)-2,3-dihydrofuro[3,2-b]pyridin-3-ol;
(R)-7-(3-(2-(5H-Pyrrolo[2,3-b]pyrazin-7-yl)thiazol-4-yl)phenyl)-6,7-dihydro-5H-pyrrolo[1,2-a]imidazol-7-ol;
(R)-7-(3-(4-(5H-Pyrrolo[2,3-b]pyrazin-7-yl)thiazol-2-yl)phenyl)-6,7-dihydro-5H-pyrrolo[1,2-a]imidazol-7-ol;
(S)-7-(3-(2-(1H-Pyrrolo[2,3-b]pyridin-3-yl)thiazol-4-yl)phenyl)-6,7-dihydro-5H-cyclopenta[b]pyridin-7-ol;
(S)-7-(3-(2-(5H-Pyrrolo[2,3-b]pyrazin-7-yl)thiazol-4-yl)phenyl)-7H-pyrrolo[1,2-a]imidazol-7-ol;
(R)-3-(3-(2-(5H-Pyrrolo[2,3-b]pyrazin-7-yl)thiazol-4-yl)phenyl)-3-hydroxy-1-methylazetidin-2-one;
(R)-3-(3-(2-(5H-Pyrrolo[2,3-b]pyrazin-7-yl)thiazol-4-yl)phenyl)-3-hydroxy-1-methylpiperidin-2-one and pharmaceutically acceptable salts thereof.
Additional illustrative embodiments of the invention are compounds selected from
(R)-4-(3-(2-(1H-Pyrrolo[2,3-b]pyridin-3-yl)thiazol-4-yl)phenyl)-5,6-dihydro-4H-cyclopenta[d]thiazol-4-ol;
(R)-3-(3-(2-(1H-pyrrolo[2,3-b]pyridin-3-yl)thiazol-5-yl)phenyl)-3-hydroxy-1-methylpyrrolidin-2-one;
(R)-3-(3-(2-(5H-Pyrrolo[2,3-b]pyrazin-7-yl)thiazol-4-yl)phenyl)-2,3-dihydrofuro[3,2-b]pyridin-3-ol;
(R)-7-(3-(2-(5H-Pyrrolo[2,3-b]pyrazin-7-yl)thiazol-4-yl)phenyl)-6,7-dihydro-5H-pyrrolo[1,2-a]imidazol-7-ol;
(R)-7-(3-(4-(5H-Pyrrolo[2,3-b]pyrazin-7-yl)thiazol-2-yl)phenyl)-6,7-dihydro-5H-pyrrolo[1,2-a]imidazol-7-ol;
(S)-7-(3-(2-(1H-Pyrrolo[2,3-b]pyridin-3-yl)thiazol-4-yl)phenyl)-6,7-dihydro-5H-cyclopenta[b]pyridin-7-ol and pharmaceutically acceptable salts thereof.
Additional illustrative embodiments of the invention are compounds selected from
(R)-4-(3-(2-(1H-Pyrrolo[2,3-b]pyridin-3-yl)thiazol-4-yl)phenyl)-5,6-dihydro-4H-cyclopenta[d]thiazol-4-ol;
(R)-3-(3-(2-(5H-Pyrrolo[2,3-b]pyrazin-7-yl)thiazol-4-yl)phenyl)-2,3-dihydrofuro[3,2-b]pyridin-3-ol;
(R)-7-(3-(2-(5H-Pyrrolo[2,3-b]pyrazin-7-yl)thiazol-4-yl)phenyl)-6,7-dihydro-5H-pyrrolo[1,2-a]imidazol-7-ol;
(R)-7-(3-(4-(5H-Pyrrolo[2,3-b]pyrazin-7-yl)thiazol-2-yl)phenyl)-6,7-dihydro-5H-pyrrolo[1,2-a]imidazol-7-ol;
(S)-7-(3-(2-(1H-Pyrrolo[2,3-b]pyridin-3-yl)thiazol-4-yl)phenyl)-6,7-dihydro-5H-cyclopenta[b]pyridin-7-ol;
(R)-3-(3-(2-(5H-Pyrrolo[2,3-b]pyrazin-7-yl)thiazol-4-yl)phenyl)-3-hydroxy-1-methylazetidin-2-one and pharmaceutically acceptable salts thereof.

Additional illustrative embodiments of the invention are compounds selected from
(R)-4-(3-(2-(1H-Pyrrolo[2,3-b]pyridin-3-yl)thiazol-4-yl)phenyl)-5,6-dihydro-4H-cyclopenta[d]thiazol-4-ol;
(R)-3-(3-(2-(1H-pyrrolo[2,3-b]pyridin-3-yl)thiazol-5-yl)phenyl)-3-hydroxy-1-methylpyrrolidin-2-one;
(R)-3-(3-(2-(5H-Pyrrolo[2,3-b]pyrazin-7-yl)thiazol-4-yl)phenyl)-2,3-dihydrofuro[3,2-b]pyridin-3-ol;
(R)-7-(3-(2-(5H-Pyrrolo[2,3-b]pyrazin-7-yl)thiazol-4-yl)phenyl)-6,7-dihydro-5H-pyrrolo[1,2-a]imidazol-7-ol;
(S)-7-(3-(2-(1H-Pyrrolo[2,3-b]pyridin-3-yl)thiazol-4-yl)phenyl)-6,7-dihydro-5H-cyclopenta[b]pyridin-7-ol;
(R)-3-(3-(2-(5H-Pyrrolo[2,3-b]pyrazin-7-yl)thiazol-4-yl)phenyl)-3-hydroxy-1-methylazetidin-2-one and pharmaceutically acceptable salts thereof.

Additional illustrative embodiments of the invention are compounds selected from
(R)-3-(3-(2-(1H-pyrrolo[2,3-b]pyridin-3-yl)thiazol-5-yl)phenyl)-3-hydroxy-1-methylpyrrolidin-2-one;
(R)-7-(3-(4-(5H-Pyrrolo[2,3-b]pyrazin-7-yl)thiazol-2-yl)phenyl)-6,7-dihydro-5H-pyrrolo[1,2-a]imidazol-7-ol;
(S)-7-(3-(2-(5H-Pyrrolo[2,3-b]pyrazin-7-yl)thiazol-4-yl)phenyl)-7H-pyrrolo[1,2-a]imidazol-7-ol;
(R)-3-(3-(2-(5H-Pyrrolo[2,3-b]pyrazin-7-yl)thiazol-4-yl)phenyl)-3-hydroxy-1-methylpiperidin-2-one and pharmaceutically acceptable salts thereof.

Additional illustrative embodiments of the invention are compounds selected from
(R)-4-(3-(2-(1H-Pyrrolo[2,3-b]pyridin-3-yl)thiazol-4-yl)phenyl)-5,6-dihydro-4H-cyclopenta[d]thiazol-4-ol;
(R)-3-(3-(2-(5H-Pyrrolo[2,3-b]pyrazin-7-yl)thiazol-4-yl)phenyl)-2,3-dihydrofuro[3,2-b]pyridin-3-ol;
(R)-7-(3-(2-(5H-Pyrrolo[2,3-b]pyrazin-7-yl)thiazol-4-yl)phenyl)-6,7-dihydro-5H-pyrrolo[1,2-a]imidazol-7-ol;
(S)-7-(3-(2-(1H-Pyrrolo[2,3-b]pyridin-3-yl)thiazol-4-yl)phenyl)-6,7-dihydro-5H-cyclopenta[b]pyridin-7-ol;
(R)-3-(3-(2-(5H-Pyrrolo[2,3-b]pyrazin-7-yl)thiazol-4-yl)phenyl)-3-hydroxy-1-methylazetidin-2-one and pharmaceutically acceptable salts thereof.

An additional illustrative embodiment is (R)-4-(3-(2-(1H-Pyrrolo[2,3-b]pyridin-3-yl)thiazol-4-yl)phenyl)-5,6-dihydro-4H-cyclopenta[d]thiazol-4-ol and pharmaceutically acceptable salts thereof.

An additional illustrative embodiment is (R)-3-(3-(2-(5H-Pyrrolo[2,3-b]pyrazin-7-yl)thiazol-4-yl)phenyl)-2,3-dihydrofuro[3,2-b]pyridin-3-ol and pharmaceutically acceptable salts thereof.

An additional illustrative embodiment is (R)-7-(3-(2-(5H-Pyrrolo[2,3-b]pyrazin-7-yl)thiazol-4-yl)phenyl)-6,7-dihydro-5H-pyrrolo[1,2-a]imidazol-7-ol and pharmaceutically acceptable salts thereof.

An additional illustrative embodiment is (S)-7-(3-(2-(1H-Pyrrolo[2,3-b]pyridin-3-yl)thiazol-4-yl)phenyl)-6,7-dihydro-5H-cyclopenta[b]pyridin-7-ol and pharmaceutically acceptable salts thereof.

An additional illustrative embodiment is (R)-3-(3-(2-(5H-Pyrrolo[2,3-b]pyrazin-7-yl)thiazol-4-yl)phenyl)-3-hydroxy-1-methylazetidin-2-one and pharmaceutically acceptable salts thereof.

Additional illustrative embodiments include the compounds in Table 1, Table 2, Table 3, and Table 4 or a pharmaceutically acceptable salt thereof.

Additional illustrative embodiments are compounds

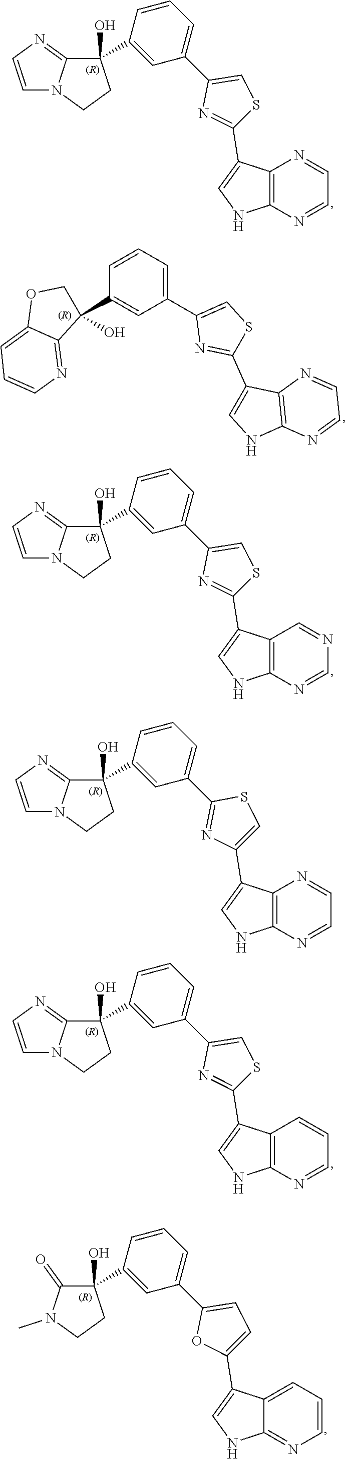

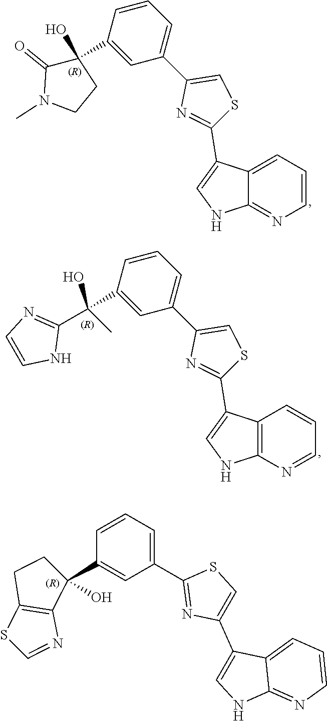

or a pharmaceutically acceptable salt thereof.

In some embodiments, the compound is

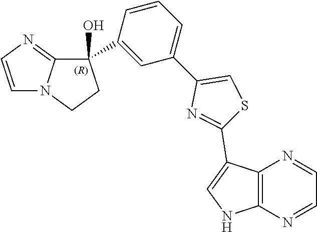

or a pharmaceutically acceptable salt thereof.

In some embodiments, the compound is

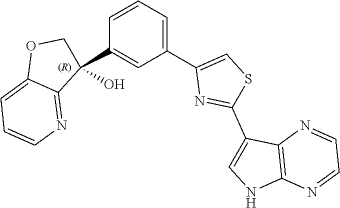

or a pharmaceutically acceptable salt thereof.

In some embodiments, the compound is

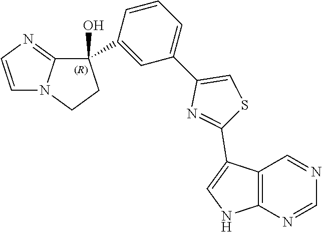

or a pharmaceutically acceptable salt thereof.

In some embodiments, the compound is

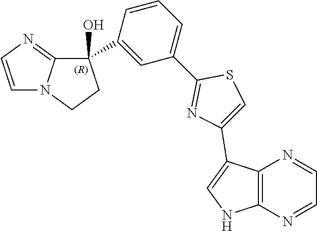

or a pharmaceutically acceptable salt thereof.

In some embodiments, the compound is

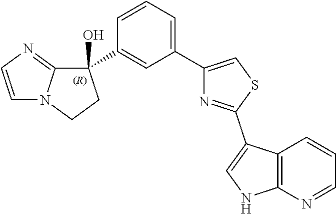

or a pharmaceutically acceptable salt thereof.

In some embodiments, the compound is

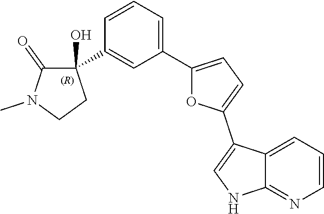

or a pharmaceutically acceptable salt thereof.

In some embodiments, the compound is

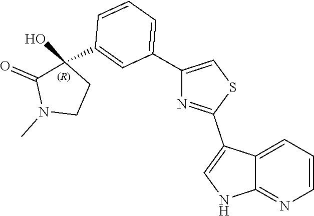

or a pharmaceutically acceptable salt thereof.

In some embodiments, the compound is

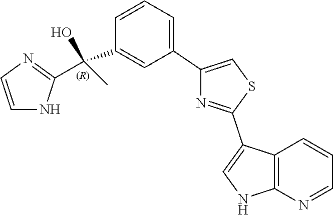

or a pharmaceutically acceptable salt thereof.

In some embodiments, the compound is

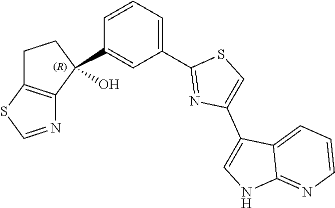

or a pharmaceutically acceptable salt thereof.

Additional illustrative embodiments are pharmaceutical compositions comprising a therapeutically effective amount of at least one compound of Formula I or pharmaceutically acceptable salt thereof.

Additional illustrative embodiments are pharmaceutical compositions comprising a therapeutically effective amount of at least one compound as shown in Table 1, Table 2, Table 3, and Table 4 or pharmaceutically acceptable salt thereof.

Additional illustrative embodiments are methods of treating a subject suffering from or diagnosed with a disease, disorder, or medical condition mediated by NIK activity, comprising administering to a subject in need of such treatment a therapeutically effective amount of at least one compound of Formula I or a pharmaceutically acceptable salt thereof.

Additional illustrative embodiments are methods of treating a subject suffering from or diagnosed with a disease, disorder, or medical condition mediated by NIK activity wherein the disease, disorder or medical condition is selected from the group consisting of cancer, inflammatory disorders, autoimmune disorders, immunodermatologic disorders and metabolic disorders.

Additional illustrative embodiments are methods of treating a subject suffering from or diagnosed with a disease, disorder, or medical condition mediated by NIK activity wherein the disease, disorder or medical condition is selected from the group consisting of systemic lupus erythematosus, rheumatoid arthritis, Graft-Versus-Host Disease, transplant rejection, Sjogren's Syndrome, pemphigus vulgaris, palmoplantar pustulosis, hidradenitis suppurativa, obesity and diabetes.

Additional illustrative embodiments are methods of treating a subject suffering from or diagnosed with a disease, disorder, or medical condition mediated by NIK activity wherein the disease, disorder or medical condition is a haematological malignancy or solid tumor.

Additional illustrative embodiments are methods of treating a subject suffering from or diagnosed with a disease, disorder, or medical condition mediated by NIK activity wherein said haematological malignancy is selected from the group consisting of multiple myeloma, Hodgkin lymphoma, T-cell leukaemia, mucosa-associated lymphoid tissue lymphoma, diffuse large B-cell lymphoma and mantle cell lymphoma.

Additional illustrative embodiments are methods of treating a subject suffering from or diagnosed with a disease, disorder, or medical condition mediated by NIK activity wherein said the solid tumor is selected from the group consisting of pancreatic cancer, breast cancer, melanoma and non-small cell lung cancer.

Illustrative compounds useful in methods of this invention are described below by reference to the illustrative synthetic schemes (“Schemes”) and specific examples for their preparation.

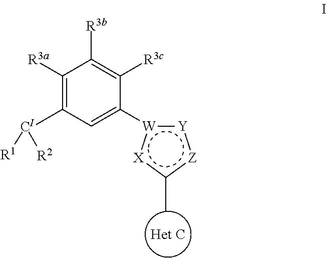

By way of illustration, but not as a limitation compounds of Formula I are prepared according to the following general preparation procedures given by Schemes 1-3. One of ordinary skill in the art will recognize that, to obtain the various compounds herein, starting materials may be suitably selected so that the ultimately desired substituents will be carried through the reaction scheme with or without protection as appropriate to yield the desired product. Alternatively, in the place of the ultimately desired substituent, a suitable group that may be carried through the reaction scheme and replaced as appropriate with the desired substituent. Unless otherwise specified, the variables in Schemes 1-3 are as defined above in reference to Formula I.

The following specific examples are provided to further illustrate embodiments within the scope of the invention.

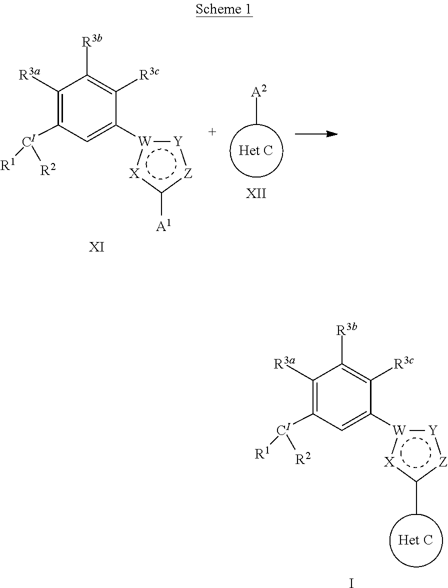

Compounds of Formula I can be prepared for example as shown in Scheme 1. The coupling of compound XI with compound XII provides compound of Formula I. When A¹ in compound XI is a boron-based coupling agent such as a boronic acid or a boronic ester and when A² in compound XII is a halide such as Br or I, this coupling is achieved by reaction under Suzuki conditions. Alternatively, when A¹ in compound XI is a halide such as Br or I and when A² in compound XII is a boron-based coupling agent such as a borolane, a boronic acid or a boronate, reaction under Suzuki conditions provides compound of Formula I. Typical Suzuki coupling conditions involve the use of a palladium catalyst and a suitable solvent. Examples of suitable palladium catalysts include, but are not limited to, PdCl₂(PPh₃)₂, Pd(Ph₃)₄, and PdCl₂(dppf). Suitable bases include, but are not limited to, K₂CO₃ and Na₂CO₃. Suitable solvents include, but are not limited to, tetrahydrofuran, toluene, and ethanol.

Stille coupling conditions can be used to couple compound XI when A1 is a stannyl group such as trimethylstannyl and compound XII when A2 is a halide such as bromide. Stille coupling conditions involve the use of a catalyst (usually palladium, but sometimes nickel), a suitable solvent, and other optional reagents such as CsF. Examples of suitable catalysts include, but are not limited to, PdCl₂(PPh₃)₂, Pd(Ph₃)₄, Pd(t-Bu₃P)₂ and PdCl₂(dppf). Suitable solvents include, but are not limited to, tetrahydrofuran, toluene, 1,4-dioxane and dimethylformamide. This reaction may be heated to a temperature of about 80-120° C.

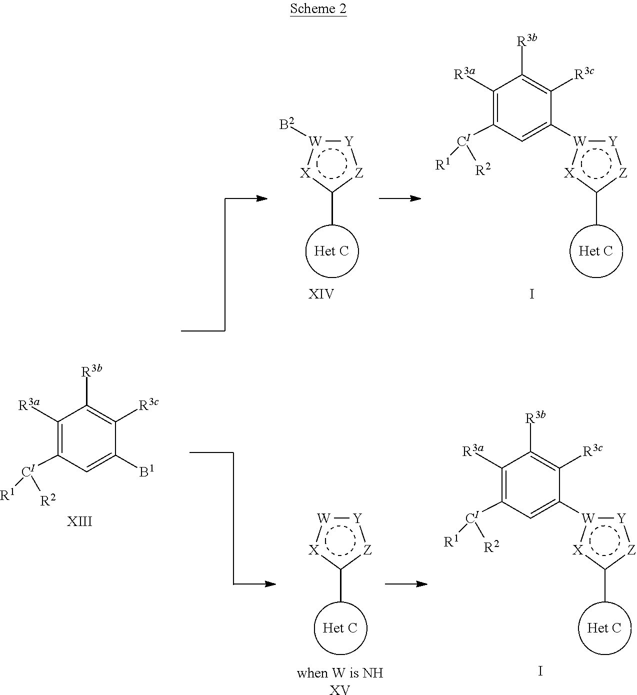

Compounds of Formula I can also be prepared for example as shown in Scheme 2. The coupling of compound XIII with compound XIV provides compound of Formula I. This is accomplished using Suzuki conditions where B¹ in compound XIII is boron-based coupling agent such as a boronic acid or a boronic ester and where B² in compound XIV is a halide. When B1 in compound XIII is an iodine atom, compound XIII can be coupled with compound XV, where W is NH in a metal catalyzed coupling using cooper (I) iodide, Cs₂CO₂ and 1,10-phenanthroline in a solvent such as DMF, THF or 1,4-dioxane. Alternatively, Buchwald or Buchwald-Hartwig coupling conditions could be used to affect this coupling. Buchwald or Buchwald-Hartwig coupling conditions involve the use of a catalyst (usually palladium, but sometimes other metals), a base, and a suitable solvent. Examples of suitable catalysts include but are not limited to (Pd[P(o-Tolyl)₃]₂), Pd₂(dba)₃ and Pd(dba)₂. Suitable solvents include, but are not limited to, toluene, dioxane, and THF. Optional bases include NaOtBu or LiHMDS. Sometimes, a bidentate phosphate ligand, such as BINAP, DPPF, BrettPhos or RuPhos can also be included.

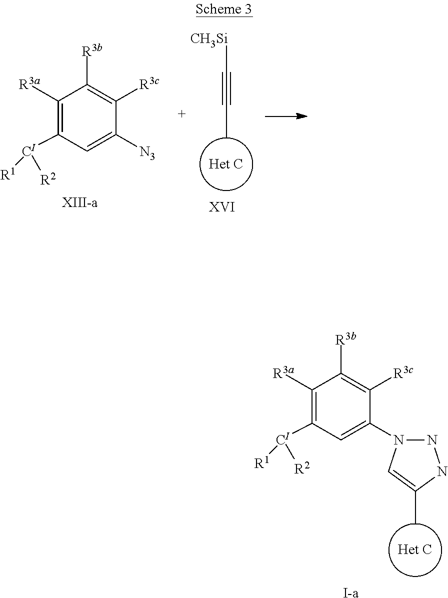

Compound of Formula I-a can be prepared for example as shown in Scheme 3. The coupling of compound XIII-a with compound XVI provides compound of Formula I-a. This coupling is accomplished by mixing compound XIII-a and compound XVI in a solvent such as DMSO at a temperature of about room temperature.

In obtaining the compounds described in the examples below and the corresponding analytical data, the following experimental and analytical protocols were followed unless otherwise indicated.

Unless otherwise specified, reaction solutions were stirred at room temperature under a N_{2(g)} or Ar_{(g)} atmosphere. When solutions were “concentrated to dryness”, they were concentrated using a rotary evaporator under reduced pressure, when solutions were dried, they are typically dried over a drying agent such as MgSO₄ or Na₂SO₄. Normal phase flash column chromatography (FCC) was performed on silica gel with prepackaged silica gel columns, such as RediSep®, using ethyl acetate (EtOAc)/hexanes, CH₂Cl₂/MeOH, or CH₂Cl₂/10% 2N NH₃ in MeOH, as eluent, unless otherwise indicated.

Thin-layer chromatography was performed using silica gel plates, such as Merck silica gel 60 F₂₅₄ 2.5 cm×7.5 cm 250 μm or 5.0 cm×10.0 cm 250 μm pre-coated silica gel plates. Preparative thin-layer chromatography was performed using silica gel plates such as EM Science silica gel 60 F₂₅₄ 20 cm×20 cm 0.5 mm pre-coated plates with a 20 cm×4 cm concentrating zone. Microwave reactions were carried out in a microwave reactor, such as a CEM Discover®, a Biotage Initiator™ or Optimizer™ microwave, at specified temperatures. Mass spectra were obtained on a mass spectrometer, such as Agilent series 1100 MSD using electrospray ionization (ESI) in positive mode unless otherwise indicated. Calculated mass corresponds to the exact mass. NMR spectra were obtained on an NMR spectrometer, such as a Bruker model DPX400 (400 MHz), DPX500 (500 MHz), DRX600 (600 MHz) spectrometer. The format of the ¹H NMR data below is as follows: Chemical shift in ppm down field of the tetramethylsilane reference (multiplicity, coupling constant J in Hz, integration).

Whenever a yield is given as a percentage, such yield refers to a mass of the entity for which the yield is given with respect to the maximum amount of the same entity that could be obtained under the particular stoichiometric conditions. Reagent concentrations that are given as percentages refer to mass ratios, unless indicated differently. Whether expressly indicated or not, yields given in the following examples are computed with respect to the dried form of the compound for which any such yield is given.

Chemical names were generated using ChemDraw Ultra 17.1 (CambridgeSoft Corp., Cambridge, MA) or OEMetaChem V1.4.0.4 (Open Eye).

Abbreviations and acronyms used herein include the following as shown below:

TABLE 5
Abbreviations and acronyms defined

| | |
|---|---|
| Ac | acyl or acetyl |
| ACN or CH₃CN | acetonitrile |
| HOAc | acetic acid |
| br | broad |
| n-BuLi | n-butyllithium |
| Bu | butyl |
| t-BuOK | potassium tert-butoxide |
| d | doublet |
| DBU | 1,8-Diazabicyclo(5.4.0)undec-7-ene |
| DCM | dichloromethane |
| DIPEA | N,N-diisopropylethylamine |
| DMAP | 4-dimethylaminopyridine |
| DMF | N,N-dimethylformamide |
| DMSO | dimethyl sulfoxide |
| ESI | electrospray ionization |
| Et | ethyl |
| Et₃N | triethylamine |
| Et₂NH | diethylamine |
| Et₂O | diethyl ether |
| EtOAc | ethyl acetate |
| EtOH | ethanol |
| FCC | flash column chromatography |
| h | hour(s) |
| HPLC | high pressure liquid chromatography |
| Hz | Hertz |
| IPA or i-PrOH | isopropanol |
| LCMS | liquid chromatography mass spectrometry |
| KOAc | potassium acetate |
| LDA | lithium diisopropylamide |
| mmol | millimoles |
| m/z | mass-to-charge ratio |
| M+ | parent molecular ion |
| Me | methyl |
| MeOH | methanol |
| min | minute(s) |
| MS | mass spectrometry |
| NaHMDS | Sodium bis(trimethylsilyl)amide |
| NaSO₂Me | Sodium methyl sulfate |
| NBS | N-bromosuccinimide |
| NMR | nuclear magnetic resonance |
| nt | not tested |
| Pd₂(dba)₃ | |
| Pd(dppf)Cl₂ or | [1,1′- |
| PdCl₂(dppf) | bis(diphenylphosphino)ferrocene]dichloropalladium(II) |
| Pd(dtbpf)Cl₂ | [1,1-bis(di-tert- |
| | butylphosphino)ferrocene]dichloropalladium(II) |
| Pd(PPh₃)₄ | tetrakis(triphenylphosphine)palladium(0) |
| Pd(PPh₃)₂Cl₂ | Bis(triphenylphosphine)palladium chloride |
| Pd(OAc)₂ | palladium(II)acetate |
| Pd(t-Bu₃P)₂ | bis(tri-tert-butylphosphine)palladium(0) |
| rt | room temperature |
| SFC | supercritical fluid chromatography |
| TBAF | tetrabutylammonium fluoride |
| TBSCl | tert-butyldimethylsilyl chloride |
| t-BuOK | potassium tert-butoxide |
| THF | tetrahydrofuran |
| TLC | thin layer chromatography |
| TosCl or TcCl | p-toluenesulfonyl chloride |
| v/v | volume-to-volume ratio |
| Xphos-Pd-G2 | Chloro(2-dicyclohexylphosphino-2′,4′,6′- |
| | triisopropyl-1,1′-biphenyl)[2-(2′-amino-1,1′- |
| | biphenyl)]palladium(II), X-Phos |
| | aminobiphenyl palladium chloride precatalyst |

### Intermediate 1. (R,S)-7-(3-(4,4,5,5-Tetramethyl-1,3,2-dioxaborolan-2-yl)phenyl)-6,7-dihydro-5H-cyclopenta[b]pyridin-7-ol

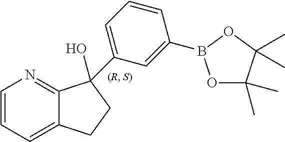 [SMILES: CC1(C)OB(c2cccc(C3(O)CCc4cccnc43)c2)OC1(C)C]

Step A. 7-(3-Bromophenyl)-6,7-dihydro-5H-cyclopenta[b]pyridin-7-ol. A solution of 1,3-dibromobenzene (1.2 g, 5.1 mmol) and anhydrous THF (12 mL) was cooled to −78° C. and n-BuLi (2.0 mL, 2.5 M in hexane; 5.0 mmol) was added drop wise. The resultant mixture was stirred at −78° C. for 30 min and then treated with 5H-cyclopenta[b]pyridin-7(6H)-one (0.50 g, 3.8 mmol) and anhydrous THF (3 mL). The temperature was raised to −50° C. and maintained at that temperature for another 30 min. The mixture was then quenched with aqueous saturated NH₄Cl solution (50 mL) and extracted with ethyl acetate (40 mL×3). The combined organic solvent extracts were washed with brine (30 mL), dried over anhydrous Na₂SO₄, filtered, and concentrated to dryness under reduced pressure. The material was purified by FCC (eluent: petroleum ether:ethyl acetate=1:0 to 3:1) to yield 7-(3-bromophenyl)-6,7-dihydro-5H-cyclopenta[b]pyridin-7-ol (860 mg, 79%) as a colorless sticky oil. ¹H NMR (400 MHz, CDCl₃) δ 8.43 (d, J=4.0 Hz, 1H), 7.68-7.61 (m, 1H), 7.46 (s, 1H), 7.39-7.33 (m, 1H), 7.23-7.18 (m, 1H), 7.17-7.10 (m, 2H), 3.59 (br s, 1H), 3.15-3.02 (m, 1H), 2.98-2.85 (m, 1H), 2.60-2.43 (m, 2H).

Step B. 7-(3-(4,4,5,5-tetramethyl-1,3,2-dioxaborolan-2-yl)phenyl)-6,7-dihydro-5H-cyclopenta[b]pyridin-7-ol. To a microwave vial containing 7-(3-bromophenyl)-6,7-dihydro-5H-cyclopenta[b]pyridin-7-ol (760 mg, 2.6 mmol), bis(pinacolato)diboron (998 mg, 3.93 mmol), and KOAc (386 mg, 3.93 mmol) was added 1,4-dioxane (20 mL). The mixture was sparged with N₂ for 5 minutes and then treated with Pd(dppf)Cl₂ (192 mg, 0.262 mmol). The mixture was sparged with N₂ for another 5 minutes and then subjected to microwave irradiation at 130° C. for 1 h. The mixture was cooled to room temperature, concentrated to dryness, and purified by FCC (eluent: petroleum ether ethyl acetate=1:0 to 0:1) to yield 7-(3-(4,4,5,5-tetramethyl-1,3,2-dioxaborolan-2-yl)phenyl)-6,7-dihydro-5H-cyclopenta[b]pyridin-7-ol (740 mg, 68%) as a pale-yellow sticky oil. LCMS (ESI): mass calcd. for C₂₀H₂₄BNO₃, 337.18; m/z found, 338.2 [M+H]⁺.

### Intermediate 2. Bromo-2-(1-(phenylsulfonyl)-1H-pyrrolo[2,3-b]pyridin-3-yl)thiazole

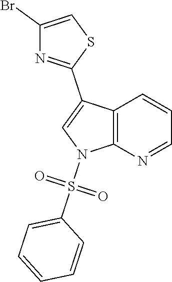 [SMILES: O=S(=O)(c1ccccc1)n1cc(-c2nc(Br)cs2)c2cccnc21]

1-(Phenylsulfonyl)-3-(4,4,5,5-tetramethyl-1,3,2-dioxaborolan-2-yl)-1H-pyrrolo[2,3-b]pyridine (5.0 g, 13 mmol), 2,4-dibromothiazole (4.8 g, 20 mmol), and Cs₂CO₃ (12.8 g, 39.3 mmol) were added to a flask and the resultant mixture dissolved in 1,4-dioxane (100 mL) and H₂O (25 mL). The mixture was sparged with Ar for 5 minutes and then treated with Pd(dppf)Cl₂ (952 mg, 1.30 mmol). The mixture was sparged with Ar for an additional minutes and then heated at 80° C. for 16 h. The mixture was cooled to room temperature and the suspension was filtered through a pad of diatomaceous earth and the pad washed with ethyl acetate (50 mL×2). The filtrate was concentrated to dryness under reduced pressure and initially purified by FCC (eluent: petroleum ether:ethyl acetate=0:1 to 5:1). This material was further purified by preparative HPLC using an HPLC column, such as a YMC-Triart Prep C18 250×50 mm×10 μm column (eluent: 35% to 85%, CH₃CN and H₂O (with 0.225% HCOOH)). The product was then suspended in water (20 mL) and the mixture was frozen by insertion into a −78° C. bath. The frozen mixture was then lyophilized to yield 4-bromo-2-(1-(phenylsulfonyl)-1H-pyrrolo[2,3-b]pyridin-3-yl)thiazole (2.5 g, 45%) as a brown solid. LCMS (ESI): mass calcd. for C₁₆H₁₀BrN₃O₂S₂, 420.09; m/z found, 421.95 [M+H]⁺. ¹H NMR (400 MHz, DMSO-d₆): δ 8.70 (5, 1H), 8.58-8.52 (m, 1H), 8.52-8.48 (m, 1H), 8.22 (d, J=7.8 Hz, 2H), 7.92 (s, 1H), 7.80-7.72 (m, 1H), 7.68-7.64 (m, 2H), 7.53-7.46 (m, 1H).

### Intermediate 3 (R,S)-7-(3-(2-(1-(Phenylsulfonyl)-1H-pyrrolo[2,3-b]pyridin-3-yl)thiazol-4-yl)phenyl)-6,7-dihydro-5H-cyclopenta[b]pyridin-7-ol

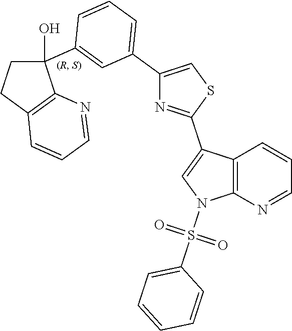 [SMILES: O=S(=O)(c1ccccc1)n1cc(-c2nc(-c3cccc(C4(O)CCc5cccnc54)c3)cs2)c2cccnc21]

7-(3-(4,4,5,5-Tetramethyl-1,3,2-dioxaborolan-2-yl)phenyl)-6,7-dihydro-5H-cyclopenta[b]pyridin-7-ol (Intermediate 1, 447 mg, 1.07 mmol), 4-bromo-2-(1-(phenylsulfonyl)-1H-pyrrolo[2,3-b]pyridin-3-yl)thiazole (Intermediate 2, 530 mg, 0.90 mmol), and K₃PO₄ (570 mg, 2.7 mmol) were added to a flask containing 1,4-dioxane (30 mL) and H₂O (8 mL). The mixture was sparged with N₂ for 5 minutes and then treated with Pd(dtbpf)Cl₂ (58.3 mg, 0.090 mmol). The mixture was sparged again with N₂ for an additional 5 minutes and then heated at 85° C. for 12 h. The mixture was cooled to room temperature, diluted with ethyl acetate (20 mL) and filtered through a pad of diatomaceous earth. The filtrate was concentrated to dryness under reduced pressure and purified by FCC (eluent: petroleum ether:ethyl acetate=1:0 to 0:1) to yield 7-(3-(2-(1-(phenylsulfonyl)-1H-pyrrolo[2,3-b]pyridin-3-yl)thiazol-4-yl)phenyl)-6,7-dihydro-5H-cyclopenta[b]pyridin-7-ol (310 mg, 63%) as pale yellow solid. LCMS (ESI): mass calcd. for C₃₀H₂₂N₄O₃S₂, 550.65; m/z found, 551.2 [M+H]⁺. ¹H NMR (400 MHz, CDCl₃) δ 8.65-8.59 (m, 1H), 8.55-8.50 (m, 2H), 8.31-8.23 (m, 3H), 7.99-7.94 (m, 1H), 7.88 (d, J=8.0 Hz, 1H), 7.69 (d, J=7.2 Hz, 1H), 7.63-7.57 (m, 1H), 7.54-7.48 (m, 2H), 7.43 (s, 1H), 7.43-7.38 (m, 1H), 7.32 (dd, J=8.0, 4.8 Hz, 1H), 7.26-7.21 (m, 2H), 3.27 (br s, 1H), 3.19-3.08 (m, 1H), 3.05-2.91 (m, 1H), 2.66-2.53 (m, 2H).

### Intermediate 4. (R,S)-3-Hydroxy-1-methyl-3-(3-(4,4,5,5-tetramethyl-1,3,2-dioxaborolan-2-yl)phenyl)pyrrolidin-2-one

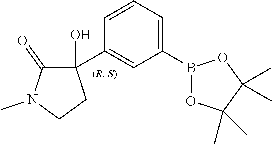

Step A: (R,S)-3-(3-Bromophenyl)-3-hydroxy-1-methylpyrrolidin-2-one. To a flask containing 1,3-dibromobenzene (1.0 mL, 2.0 g, 8.3 mmol) was added diethyl ether (50 mL) and the solution was cooled to −78° C. Then, n-BuLi (4.15 mL, 2.5 M in hexanes) was added which afforded a colorless homogeneous solution. After stirring at −78° C.; for 30 min, 1-methylpyrrolidine-2,3-dione (1.21 g, 9.97 mmol, in 20 mL THF) was added slowly over 5 min. Once the addition of 1-methylpyrrolidine-2,3-dione was complete, the −78° C.; bath was replaced with an ice-water bath and the reaction mixture was then allowed to warm to room temperature slowly over 1 h. After 4 h, the reaction mixture was quenched with aqueous saturated NH₄Cl solution and extracted with EtOAc (5×40 mL). The combined organic solvent extracts were washed with brine, dried over MgSO₄, filtered and concentrated. The material was purified by FCC (100% DCM/0% EtOAC to 0% DCM/100% EtOAc) to yield (R,S)-3-(3-bromophenyl)-3-hydroxy-1-methylpyrrolidin-2-one (710 mg) as an off-white solid. MS (ESI): mass calcd. for C₁₁H₁₂BrNO₂, 270.13; m/z found, 271.05 [M+H]⁺. ¹H NMR (400 MHz, CDCl₃) δ 7.54 (d, J=1.4 Hz, 1H), 7.41 (d, J=7.8 Hz, 1H), 7.31-7.24 (m, 1H), 7.24-7.14 (m, 1H), 3.48-3.40 (m, 1H), 3.39-3.28 (m, 1H), 2.99 (s, 3H), 2.50-2.37 (m, 1H), 2.38-2.25 (m, 1H).

Step B. (R,S)-3-Hydroxy-1-methyl-3-(3-(4,4,5,5-tetramethyl-1,3,2-dioxaborolan-2-yl)phenyl)pyrrolidin-2-one. To a flask containing (R,S)-3-(3-bromophenyl)-3-hydroxy-1-methylpyrrolidin-2-one (1.3 g, 4.8 mmol), 4,4,4′,4′,5,5,5′,5′-octamethyl-2,2′-bi(1,3,2-dioxaborolane (2.0 g, 7.9 mmol), and KOAc (1.42 g, 14.5 mmol) was added 1,4-dioxane (20 mL). The mixture was sparged with N₂ for 5 minutes and then treated with Pd(dppf)Cl₂ (352 mg, 0.481 mmol). The resultant mixture was heated at 70° C. for 16 h and then cooled to room temperature. The suspension was filtered through a pad of diatomaceous earth and the pad was rinsed with ethyl acetate (80 mL). The filtrate was concentrated to dryness under reduced pressure and purified by FCC (eluent: petroleum ether:ethyl acetate=10:1 to 1:2) to yield (R,S)-3-hydroxy-1-methyl-3-(3-(4,4,5,5-tetramethyl-1,3,2-dioxaborolan-2-yl)phenyl)pyrrolidin-2-one (1.4 g, 82%) as a brown solid. LCMS (ESI): mass calcd. for C₁₇H₂₄BNO₄, 317.18; m/z found, 318.2 [M+H]⁺.

### Intermediate 5. 5-Bromo-2-(1-(phenylsulfonyl)-1H-pyrrolo[2,3-b]pyridin-3-yl)thiazole

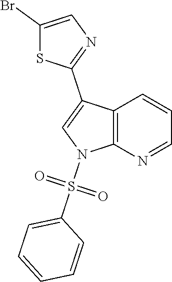 [SMILES: O=S(=O)(c1ccccc1)n1cc(-c2ncc(Br)s2)c2cccnc21]

1-(Phenylsulfonyl)-3-(4,4,5,5-tetramethyl-1,3,2-dioxaborolan-2-yl)-1H-pyrrolo[2,3-b]pyridine (1.0 g, 2.6 mmol), 2,5-dibromothiazole (676 mg, 2.79 mmol), and K₃PO₄ (1.66 g, 7.81 mmol) were dissolved in THE (20 mL). The resultant mixture was sparged with N₂ for 5 minutes and then treated with Pd(OAc)₂ (15 mg, 0.065 mmol) and 4,5-bis(diphenylphosphino)-9,9-dimethylxanthene (38 mg, 0.065 mmol). This mixture was sparged with N₂ for another 5 minutes and then heated at 80° C. for 16 h. The mixture was then cooled to room temperature, and the suspension was filtered through a pad of diatomaceous earth and concentrated to dryness under reduced pressure. The residue was purified by FCC (eluent: petroleum ether:ethyl acetate=1:0 to 3:1) to yield 5-bromo-2-(1-(phenylsulfonyl)-1H-pyrrolo[2,3-b]pyridin-3-yl)thiazole (640 mg, 54%) as a white solid. LCMS (ESI): mass calcd. for C₁₆H₁₀BrN₃O₂S₂, 418.94; m/z found, 420.0 [M+H]⁺. ¹H NMR (400 MHz, DMSO-d₆) δ 8.66 (s, 1H), 8.59-8.55 (m, 1H), 8.49-8.46 (m, 1H), 8.22-8.18 (m, 2H), 8.03 (s, 1H), 7.78-7.72 (m, 1H), 7.68-7.62 (m, 2H), 7.48-7.43 (m, 1H).

### Intermediate 6. (R,S)-3-Hydroxy-1-methyl-3-(3-(2-(1-(phenylsulfonyl)-1H-pyrrolo[2,3-b]pyridin-3-yl)thiazol-5-yl)phenyl)pyrrolidin-2-one

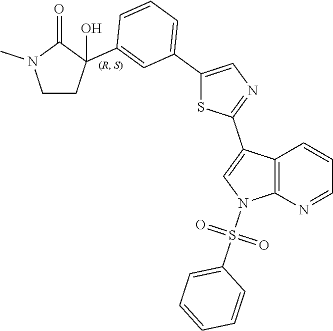 [SMILES: CN1CCC(O)(c2cccc(-c3cnc(-c4cn(S(=O)(=O)c5ccccc5)c5ncccc45)s3)c2)C1=O]

5-Bromo-2-(1-(phenylsulfonyl)-1H-pyrrolo[2,3-b]pyridin-3-yl)thiazole (Intermediate 5, 420 mg, 0.999 mmol), (R,S)-3-hydroxy-1-methyl-3-(3-(4,4,5,5-tetramethyl-1,3,2-dioxaborolan-2-yl)phenyl)pyrrolidin-2-one (Intermediate 4, 356 mg, 0.999 mmol), and K₃PO₄ (408 mg, 3.00 mmol) were added to a flask containing 1,4-dioxane (16 mL) and H₂O (4 mL). The resultant mixture was sparged with N₂ for 5 minutes and then treated with Pd(dtbpf)Cl₂ (65.1 mg, 0.100 mmol). This mixture was sparged with N₂ for another 5 minutes and then heated at 85° C. for 12 h. The mixture was then cooled to room temperature and then concentrated to dryness. The material was then diluted with H₂O (20 mL), and the aqueous portion was extracted with dichloromethane (20 mL×2). The combined organic solvent extracts were washed with brine (20 mL), dried over anhydrous Na₂SO₄, filtered, concentrated to dryness, and purified by FCC (eluent: ethyl acetate) to yield (R,S)-3-hydroxy-1-methyl-3-(3-(2-(1-(phenylsulfonyl)-1H-pyrrolo[2,3-b]pyridin-3-yl)thiazol-5-yl)phenyl)pyrrolidin-2-one (400 mg, 72%) as a yellow solid. LCMS (ESI): mass calcd. for C₂₇H₂₂N₄O₄S₂, 530.11; m/z found, 531.1 [M+H]⁺. ¹H NMR (400 MHz, DMSO-d₆) δ 8.69-8.65 (m, 1H), 8.64 (s, 1H), 8.52-8.47 (m, 1H), 8.34 (s, 1H), 8.26-8.20 (m, 2H), 7.79-7.70 (m, 2H), 7.70-7.63 (m, 3H), 7.51-7.42 (m, 2H), 7.34 (d, J=8.0 Hz, 1H), 6.16 (s, 1H), 3.51-3.43 (m, 1H), 3.43-3.35 (m, 1H), 2.86 (s, 3H), 2.43-2.34 (m, 1H), 2.31-2.23 (m, 1H).

### Intermediate 7. 4-Bromo-2-(7-tosyl-7H-pyrrolo[2,3-d]pyrimidin-5-yl)thiazole

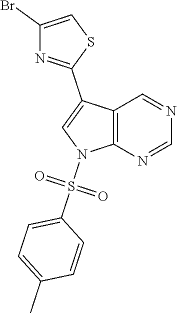 [SMILES: Cc1ccc(S(=O)(=O)n2cc(-c3nc(Br)cs3)c3cncnc32)cc1]

To 5-(4,4,5,5-tetramethyl-1,3,2-dioxaborolan-2-yl)-7-tosyl-7H-pyrrolo[2,3-d]pyrimidine (900 mg, 2.25 mmol), 2,4-dibromothiazole (821 mg, 3.38 mmol), and Cs₂CO₃ (1.84 g, 5.65 mmol) was added 1,4-dioxane (12.5 mL) and H₂O (2.5 mL). The mixture was sparged with Ar for 5 minutes and then treated with Pd(dppf)Cl₂ (165 mg, 0.226 mmol). The mixture was sparged with Ar for another 5 minutes and the resultant mixture was subjected to microwave irradiation at 80° C.; for 1 h. The mixture was cooled to room temperature, diluted with water (50 mL) and extracted with ethyl acetate (50 mL×3). The combined organic solvent extracts were dried over anhydrous Na₂SO₄, filtered, concentrated and purified by FCC (petroleum ether:ethyl acetate=20:1 to 5:1) to yield 4-bromo-2-(7-tosyl-7H-pyrrolo[2,3-d]pyrimidin-5-yl)thiazole (900 mg, 65%) as a brown solid. LCMS (ESI): mass calcd. for C₁₆H₁₁BrN₄O₂S₂, 433.95; m/z found, 434.9 [M+H]⁺.

### Intermediate 8. (R,S)-7-(3-(2-(7-Tosyl-7H-pyrrolo[2,3-d]pyrimidin-5-yl)thiazol-4-yl)phenyl)-6,7-dihydro-5H-cyclopenta[b]pyridin-7-ol

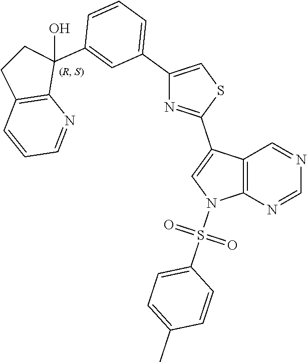 [SMILES: Cc1ccc(S(=O)(=O)n2cc(-c3nc(-c4cccc(C5(O)CCc6cccnc65)c4)cs3)c3cncnc32)cc1]

To a microwave tube containing 4-bromo-2-(7-tosyl-7H-pyrrolo[2,3-d]pyrimidin-5-yl)thiazole (Intermediate 7, 880 mg, 2.0 mmol), 7-(3-(4,4,5,5-tetramethyl-1,3,2-dioxaborolan-2-yl)phenyl)-6,7-dihydro-5H-cyclopenta[b]pyridin-7-ol (Intermediate 1, 750 mg, 2.2 mmol), and K₃PO₄ (1.29 g, 6.08 mmol) was added 1,4-dioxane (12.5 mL) and H₂O (2.5 mL). The mixture was sparged with Ar for 5 minutes and then treated with Pd(dtbpf)Cl₂ (132 mg, 0.203 mmol). The mixture was sparged with Ar for another 5 minutes and the resultant mixture was subjected to microwave irradiation for 1 h at 80° C. The mixture was then cooled to room temperature, diluted with water (80 mL) and extracted with ethyl acetate (60 mL×3). The combined organic solvent extracts were dried over anhydrous Na₂SO₄, filtered, and concentrated to dryness. The product was purified by preparative HPLC using an HPLC column, such as a Boston Green ODS C18, 150×40 mm×10 μm column (eluent: 50% to 81%, CH₃CN and H₂O (with 10 mM NH₄HCO₃)). The product was suspended in water (10 mL) and the mixture was frozen by insertion into a −78° C. bath. The frozen mixture was then lyophilized to yield (R,S)-7-(3-(2-(7-tosyl-7H-pyrrolo[2,3-d]pyrimidin-5-yl)thiazol-4-yl)phenyl)-6,7-dihydro-5H-cyclopenta[b]pyridin-7-ol (390 mg, 32%) as a brown solid. LCMS (ESI): mass calcd. for C₃₀H₂₃N₅O₃S₂, 565.12; m/z found, 566.0 [M+H]⁺. ¹H NMR (400 MHz, DMSO-d₆) δ 9.60 (s, 1H), 9.08 (s, 1H), 8.71 (s, 1H), 8.38 (d, J=4.2 Hz, 1H), 8.14-8.07 (m, 3H), 8.04 (s, 1H), 7.87 (d, J=7.6 Hz, 1H), 7.73 (d, J=7.3 Hz, 1H), 7.44 (d, J=8.1 Hz, 2H), 7.41-7.34 (m, 1H), 7.29 (d, J=7.8 Hz, 1H), 7.23 (dd, J=7.3, 4.9 Hz, 1H), 5.89 (s, 1H), 3.13-3.01 (m, 1H), 2.94-2.84 (m, 1H), 2.46-2.35 (m, 1H), 2.46-2.35 (m, 1H), 2.33 (s, 3H).

### Intermediate 9. 7-(4,4,5,5-Tetramethyl-1,3,2-dioxaborolan-2-yl)-5-tosyl-5H-pyrrolo[2,3-b]pyrazine

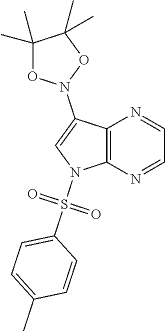 [SMILES: Cc1ccc(S(=O)(=O)n2cc(N3OC(C)(C)C(C)(C)O3)c3nccnc32)cc1]

Step A. 7-Bromo-5-tosyl-5H-pyrrolo[2,3-b]pyrazine. TosCl (9.2 g, 48 mmol) was added to a solution of 7-bromo-5H-pyrrolo[2,3-b]pyrazine (8.0 g, 40 mmol), DMAP (986 mg, 8.07 mmol), Et₃N (28.0 ml, 201 mmol), and dichloromethane (100 mL). The resultant mixture was stirred at room temperature for 12 h. This reaction mixture was concentrated under reduced pressure to yield the product, which was washed with methanol (30 mL), filtered, concentrated, and purified by FCC (eluent: ethyl acetate:methanol=1:0 to 1:10) to yield the 7-bromo-5-tosyl-5H-pyrrolo[2,3-b]pyrazine (9.0 g, 64%) as a white solid. LCMS (ESI): mass calcd. for C₁₃H₁₀BrN₃O₂S, 350.97; m/z found, 354.0 [M+H]⁺.

Step B. 7-(4,4,5,5-Tetramethyl-1,3,2-dioxaborolan-2-yl)-5-tosyl-5H-pyrrolo[2,3-b]pyrazine. Pd(dppf)Cl₂ (623 mg, 0.851 mmol) was added to a solution of 7-bromo-5-tosyl-5H-pyrrolo[2,3-b]pyrazine (3.0 g, 8.5 mmol), 4,4,4′,4′,5,5,5′,5′-octamethyl-2,2′-bi(1,3,2-dioxaborolane) (4.33 g, 17.1 mmol), KOAc (2.51 g, 25.6 mmol), and 1,4-dioxane (60 mL). The resultant mixture was heated at 70° C. for 12 h. This mixture was then cooled to room temperature and the suspension was filtered through a pad of diatomaceous earth and the pad washed with ethyl acetate (70 mL). The filtrate was concentrated to dryness and purified by FCC (eluent: petroleum ether:ethyl acetate=1:0 to 3:1) to yield 7-(4,4,5,5-tetramethyl-1,3,2-dioxaborolan-2-yl)-5-tosyl-5H-pyrrolo[2,3-b]pyrazine (1.0 g, 26%) as a white solid. LCMS (ESI): mass calcd. for C₁₉H₂₂BN₃O₄S, 399.14; m/z found 318.1 [M+H, M−81]⁺.

### Intermediate 10. 4-Bromo-2-(5-tosyl-5H-pyrrolo[2,3-b]pyrazin-7-yl)thiazole

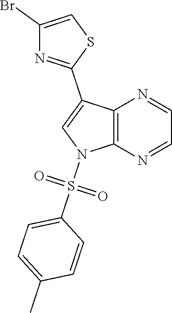 [SMILES: Cc1ccc(S(=O)(=O)n2cc(-c3nc(Br)cs3)c3nccnc32)cc1]

To a flask containing 2,4-dibromothiazole (913 mg, 3.76 mmol), Cs₂CO₃ (2.04 g, 6.26 mmol), 1,4-dioxane (10 mL), and H₂O (2 mL) was added 7-(4,4,5,5-tetramethyl-1,3,2-dioxaborolan-2-yl)-5-tosyl-5H-pyrrolo[2,3-b]pyrazine (Intermediate 9, 1.00 g, 2.51 mmol) The mixture was sparged with Ar for 5 minutes and then treated with Pd(dppf)Cl₂ (183 mg, 0.250 mmol). The mixture was sparged with Ar for another 5 minutes and subjected to microwave irradiation at 80° C. for 1 h. The mixture was cooled to room temperature, the suspension was filtered through a pad of diatomaceous earth and the pad washed with ethyl acetate (50 mL). The filtrate was concentrated to dryness under reduced pressure. The product was purified by FCC (eluent: petroleum ether:ethyl acetate=0:1 to 3:1) to yield 4-bromo-2-(5-tosyl-5H-pyrrolo[2,3-b]pyrazin-7-yl)thiazole (400 mg, 37%) as a brown solid. LCMS (ESI): mass calcd. for C₁₆H₁₁BrN₄O₂S₂, 435.31; m/z found, 437.0 [M+H]⁺.

### Intermediate 11. (R)-7-(3-Bromophenyl)-6,7-dihydro-5H-pyrrolo[1,2-a]imidazol-7-ol

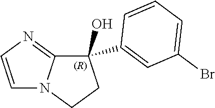

Step A. (R,S)-7-(3-Bromophenyl)-6,7-dihydro-5H-pyrrolo[1,2-a]imidazol-7-ol. To a solution of 1,3-dibromobenzene (1.00 g, 4.26 mmol) in anhydrous THE (5 mL) that had been cooled to −70° C., was added n-BuLi (1.70 mL, 2.5 M in hexane, 4.25 mmol). The mixture was then stirred at −70° C. for 30 min before 5H-pyrrolo[1,2-a]imidazol-7(6H)-one (400 mg, 3.3 mmol) was added. The mixture was stirred at −50° C. for another 30 min and then quenched with aqueous saturated NH₄Cl solution (10 mL). The mixture was warmed to room temperature and extracted with ethyl acetate (20 mL×3). The combined organic solvent extracts were dried over anhydrous Na₂SO₄, filtered, and concentrated to dryness under reduced pressure. The reaction product was initially purified by FCC (eluent: petroleum ether:ethyl acetate, 1:0 to 3:1 (wherein the ethyl acetate contained 10% methanol)) to yield (R,S)-7-(3-bromophenyl)-6,7-dihydro-5H-pyrrolo[1,2-a]imidazol-7-ol.

Step B. (R)-7-(3-bromophenyl)-6,7-dihydro-5H-pyrrolo[1,2-a]imidazol-7-ol. The enantiomers of (R,S)-7-(3-bromophenyl)-6,7-dihydro-5H-pyrrolo[1,2-a]imidazol-7-ol were separated by SFC using an SFC column, such as a DAICEL CHIRALPAK AD-H 250 mm×30 mm, 5 μm column (isocratic elution: 30%: 70%, EtOH (containing 0.1% of 25% NH₃): supercritical CO₂) yielding as a first eluting enantiomer, (R)-7-(3-bromophenyl)-6,7-dihydro-5H-pyrrolo[1,2-a]imidazol-7-ol and as a second eluting enantiomer, (S)-7-(3-bromophenyl)-6,7-dihydro-5H-pyrrolo[1,2-a]imidazol-7-ol (Intermediate 12). The fractions containing (R)-7-(3-bromophenyl)-6,7-dihydro-5H-pyrrolo[1,2-a]imidazol-7-ol were collected and concentrated under reduced pressure. This product was suspended in water (10 mL) and the mixture was frozen by insertion into a −78° C. bath. The frozen mixture was then lyophilized to yield (R)-7-(3-bromophenyl)-6,7-dihydro-5H-pyrrolo[1,2-a]imidazol-7-ol (220 mg, 21%) as a white solid. SFC Rₜ=3.845 min. LCMS (ESI): mass calcd. for C₁₂H₁₁BrN₂O, 278.01; m/z found, 279.0 [M+H]⁺. ¹H NMR (400 MHz, DMSO-d₆) δ7.69-7.65 (m, 1H), 7.50-7.45 (m, 1H), 7.44-7.39 (m, 1H), 7.33-7.28 (m, 1H), 7.19-7.14 (m, 1H), 7.03-6.96 (m, 1H), 6.21 (s, 1H), 4.15-4.08 (m, 1H), 4.07-3.99 (m, 1H), 2.80-2.74 (m, 2H).

### Intermediate 12. (S)-7-(3-Bromophenyl)-6,7-dihydro-5H-pyrrolo[1,2-a]imidazol-7-ol

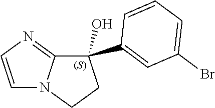

The chiral separation described for Intermediate 11 provided (S)-7-(3-bromophenyl)-6,7-dihydro-5H-pyrrolo[1,2-a]imidazol-7-ol (237 mg, 23%) as a white solid. SFC Rₜ=4.480 min. LCMS (ESI): mass calcd. for C₁₂H₁₁BrN₂O, 278.01; m/z found, 279.0 [M+H]⁺. ¹H NMR (400 MHz, DMSO-d₆) δ 7.68 (t, J=1.7 Hz, 1H), 7.50-7.45 (m, 1H), 7.44-7.39 (m, 1H), 7.35-7.29 (m, 1H), 7.18 (d, J=1.2 Hz, 1H), 7.03-6.97 (m, 1H), 6.22 (s, 1H), 4.14-4.01 (m, 2H), 2.80-2.75 (m, 2H).

### Intermediate 13. (R)-7-(3-(4,4,5,5-Tetramethyl-1,3,2-dioxaborolan-2-yl)phenyl)-6,7-dihydro-5H-pyrrolo[1,2-a]imidazol-7-ol

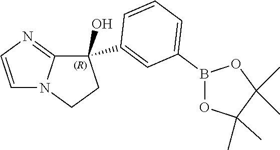

A flask containing (R)-7-(3-bromophenyl)-6,7-dihydro-5H-pyrrolo[1,2-a]imidazol-7-ol (Intermediate 11, 270 mg, 0.97 mmol), 4,4,4′,4′,5,5,5′,5′-octamethyl-2,2′-bi(1,3,2-dioxaborolane) (491 mg, 1.93 mmol), KOAc (284 mg, 2.89 mmol), and 1,4-dioxane (10 mL) was sparged with Ar for 5 minutes and then treated with Pd(dppf)Cl₂ (71 mg, 0.097 mmol). The mixture was sparged with Ar for another 5 minutes and then subjected to microwave irradiation at 100° C. for 1 h. The mixture was then allowed to cool and additional Pd(dppf)Cl₂ (35 mg, 0.048 mmol) and 4,4,4′,4′,5,5,5′,5′-octamethyl-2,2′-bi(1,3,2-dioxaborolane) (250 mg, 0.98 mmol) were added. The mixture was sparged with Ar for 5 minutes and then subjected to microwave irradiation at 100° C. for 2 h. The reaction mixture was concentrated to dryness under reduced pressure and the product was purified by FCC (eluent: petroleum ether:ethyl acetate=1:0 to 0:1) to yield (R)-7-(3-(4,4,5,5-tetramethyl-1,3,2-dioxaborolan-2-yl)phenyl)-6,7-dihydro-5H-pyrrolo[1,2-a]imidazol-7-ol (280 mg, 48%) as a brown oil. LCMS (ESI): mass calcd. for C₁₆H₂₃BN₂O₃, 326.18; m/z found, 327.2 [M+H]⁺.

### Intermediate 14. (R)-7-(3-(2-(5-Tosyl-5H-pyrrolo[2,3-b]pyrazin-7-yl)thiazol-4-yl)phenyl)-6,7-dihydro-5H-pyrrolo[1,2-a]imidazol-7-ol

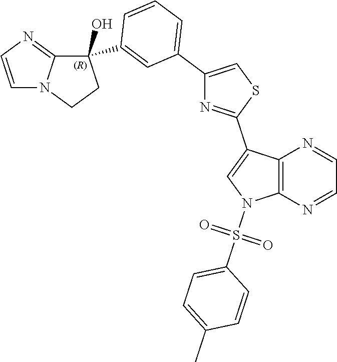

A microwave vial containing 4-bromo-2-(5-tosyl-5H-pyrrolo[2,3-b]pyrazin-7-yl)thiazole (Intermediate 10, 350 mg, 0.80 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 280 mg, 0.86 mmol), K₃PO₄ (776 mg, 3.66 mmol), 1,4-dioxane (12 mL), and H₂O (3 mL) was sparged with Ar for 5 minutes and then treated with Pd(dtbpf)Cl₂ (77 mg, 0.12 mmol). The mixture was sparged with Ar for another 5 minutes and then subjected to microwave irradiation at 80° C. for 1 h. The reaction mixture was cooled to room temperature and the suspension was filtered through a pad of diatomaceous earth which was washed with ethyl acetate (10 mL). The filtrate was poured into water H₂O (20 mL) and extracted with ethyl acetate (40 mL×3). The combined organic solvent extracts were washed with brine, dried over anhydrous Na₂SO₄, filtered, and concentrated to dryness under reduced pressure to yield the product which was purified by FCC (eluent: petroleum ether:ethyl acetate=1:0 to 0:1, then dichloromethane:methanol=1:0 to 10:1) to yield (R)-7-(3-(2-(5-tosyl-5H-pyrrolo[2,3-b]pyrazin-7-yl)thiazol-4-yl)phenyl)-6,7-dihydro-5H-pyrrolo[1,2-a]imidazol-7-ol (300 mg) as a brown solid. LCMS (ESI): mass calcd. for C₂₈H₂₂N₆O₃S₂, 554.12; m/z found 555.1 [M+H]⁺.

### Intermediate 15: (R)-3-(3-Bromophenyl)-3-hydroxy-1-methylpyrrolidin-2-one

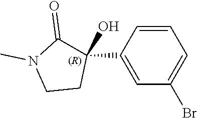

The enantiomers of (R,S)-3-(3-bromophenyl)-3-hydroxy-1-methylpyrrolidin-2-one (Intermediate 4, Step A, 3.3 g, 12 mmol) were separated by SFC using an SFC column, such as a DAICEL CHIRALPAK AD 250 mm×30 mm, 10 μm column (isocratic elution: EtOH (containing 0.1% of 25% aqueous NH₃): supercritical CO₂, 25%: 75%). The first eluting enantiomer, (R)-3-(3-bromophenyl)-3-hydroxy-1-methylpyrrolidin-2-one (1.57 g, 46%, SFC Rₜ=4.16 min) was obtained as a colorless solid. The second eluting enantiomer, (S)-3-(3-bromophenyl)-3-hydroxy-1-methylpyrrolidin-2-one (Intermediate 111) was also obtained.

### Intermediate 16. (R)-3-Hydroxy-1-methyl-3-(3-(4,4,5,5-tetramethyl-1,3,2-dioxaborolan-2-yl)phenyl)pyrrolidin-2-one

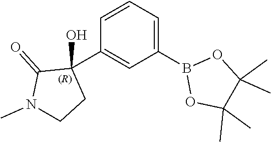

(R)-3-Hydroxy-1-methyl-3-(3-(4,4,5,5-tetramethyl-1,3,2-dioxaborolan-2-yl)phenyl)pyrrolidin-2-one was prepared in analogous fashion to Intermediate 4, except (R)-3-(3-bromophenyl)-3-hydroxy-1-methylpyrrolidin-2-one (Intermediate 15) was used in place of (R,S)-3-(3-bromophenyl)-3-hydroxy-1-methylpyrrolidin-2-one. LCMS (ESI): mass calcd. for C₁₇H₂₄BNO₄, 317.18; m/z found, 318.2 [M+1]⁺. ¹H NMR (400 MHz, DMSO-d₆): 7.75 (s, 1H), 7.57 (d, J=7.2 Hz, 1H), 7.44 (d, J=7.9 Hz, 1H), 7.37-7.31 (m, 1H), 5.97 (s, 1H), 3.47-3.39 (m, 1H), 3.31-3.26 (m, 1H), 2.83 (5, 3H), 2.31-2.17 (m, 2H), 1.30 (s, 12H);

### Intermediate 17. 4-Bromo-2-(5-methyl-1-tosyl-1H-pyrrolo[2,3-b]pyridin-3-yl)thiazole

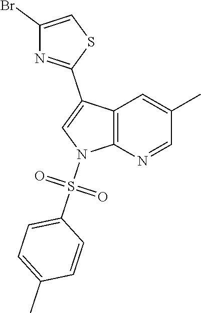 [SMILES: Cc1ccc(S(=O)(=O)n2cc(-c3nc(Br)cs3)c3cc(C)cnc32)cc1]

Step A. 3-Bromo-5-methyl-1-tosyl-1H-pyrrolo[2,3-b]pyridine. To a solution of 3-bromo-5-methyl-1H-pyrrolo[2,3-b]pyridine (1.0 g, 4.7 mmol), dimethylaminopyridine (116 mg, 0.950 mmol), triethylamine (3.3 ml, 24 mmol), and dichloromethane (20 mL) was added p-toluenesulfonyl chloride (1.1 g, 5.8 mmol). The resultant mixture was stirred at room temperature for 16 h, then poured into water (20 mL) and extracted with dichloromethane (20 mL×2). The combined organic solvent extracts were dried over anhydrous Na₂SO₄, filtered, and concentrated to dryness under reduced pressure. The product was purified by FCC (eluent: petroleum ether:ethyl acetate=1:0 to 2:1) to yield 3-bromo-5-methyl-1-tosyl-1H-pyrrolo[2,3-b]pyridine (1.7 g, 84%) as a yellow solid. ¹H NMR (400 MHz, DMSO-d₆) δ 8.29 (s, 1H), 8.14 (s, 1H), 7.98 (d, J=7.5 Hz, 2H), 7.74 (s, 1H), 7.41 (d, J=7.3 Hz, 2H), 2.38 (s, 3H), 2.33 (s, 3H).

Step B. 5-Methyl-3-(4,4,5,5-tetramethyl-1,3,2-dioxaborolan-2-yl)-1-tosyl-1H-pyrrolo[2,3-b]pyridine. To a microwave tube containing 3-bromo-5-methyl-1-tosyl-1H-pyrrolo[2,3-b]pyridine (300 mg, 0.7 mmol), 4,4,4′,4′,5,5,5′,5′-octamethyl-2,2′-bi(1,3,2-dioxaborolane) (196 mg, 0.772 mmol), and KOAc (207 mg, 2.11 mmol) was added 1,4-dioxane (10 mL). The mixture was sparged with N₂ for 5 minutes and then treated with Pd(OAC)₂ (7.9 mg, 0.035 mmol) and tricyclohexylphosphine (20 mg, 0.07 mmol). The resultant mixture was subjected to microwave irradiation at 110° C. for 16 h. After this time, the mixture was cooled to room temperature and concentrated under reduced pressure and was used directly in the next step without further purification.

Step C. 4-Bromo-2-(5-methyl-1-tosyl-1H-pyrrolo[2,3-b]pyridin-3-yl)thiazole. To a flask containing 5-methyl-3-(4,4,5,5-tetramethyl-1,3,2-dioxaborolan-2-yl)-1-tosyl-1H-pyrrolo[2,3-b]pyridine was added 1,4-dioxane (10 mL) followed by 2,4-dibromothiazole (124 mg, 0.510 mmol), Cs₂CO₃ (502 mg, 1.54 mmol), and H₂O (2 ml). The mixture was sparged with N₂ for 5 minutes and then treated with Pd(dppf)Cl₂ (38 mg, 0.052 mmol). The resultant mixture was heated at 85° C. for 2 h, then cooled to room temperature and quenched with water (10 mL). This mixture was extracted with ethyl acetate (10 mL×2), and the combined organic solvent extracts were dried over anhydrous Na₂SO₄, filtered, and concentrated to dryness under reduced pressure. The product was initially purified by FCC (eluent: petroleum ether ethyl acetate=1:0 to 2:1). To further purify the product, it was triturated with ethyl acetate (20 mL) and the suspension isolated via filtration. The filter cake was washed with ethyl acetate (10 mL) and then dried under reduced pressure to yield 4-bromo-2-(5-methyl-1-tosyl-1H-pyrrolo[2,3-b]pyridin-3-yl)thiazole (200 mg, 56%) as a gray solid. ¹H NMR (400 MHz, DMSO-d₆) δ 8.60 (s, 1H), 8.32 (d, J=4.5 Hz, 2H), 8.09-8.03 (m, 2H), 7.91 (s, 1H), 7.43 (d, J=8.3 Hz, 2H), 2.42 (s, 3H), 2.34 (s, 3H).

### Intermediate 18. (R)-3-Hydroxy-1-methyl-3-(3-(2-(5-methyl-1-tosyl-1H-pyrrolo[2,3-b]pyridin-3-yl)thiazol-4-yl)phenyl)pyrrolidin-2-one

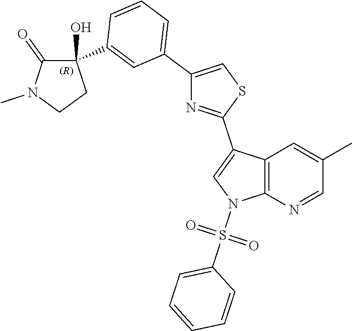

To a sealed tube containing 4-bromo-2-(5-methyl-1-tosyl-1H-pyrrolo[2,3-b]pyridin-3-yl)thiazole (Intermediate 17, 130 mg, 0.19 mmol), (R)-3-hydroxy-1-methyl-3-(3-(4,4,5,5-tetramethyl-1,3,2-dioxaborolan-2-yl)phenyl)pyrrolidin-2-one (Intermediate 16, 71 mg, 0.22 mmol), and K₃PO₄ (76 mg, 0.56 mmol) were added 1,4-dioxane (4 mL) and H₂O (1 mL). The mixture was sparged with Ar for 5 minutes and then treated with Pd(dtbpf)Cl₂ (13 mg, 0.020 mmol). The mixture was sparged with Ar for another 5 minutes and then heated at 100° C. for 2 h. The mixture was cooled to room temperature, concentrated to dryness under reduced pressure, and purified by FCC (eluent: petroleum ether ethyl acetate=1:0 to 0:1) to yield (R)-3-hydroxy-1-methyl-3-(3-(2-(5-methyl-1-tosyl-1H-pyrrolo[2,3-b]pyridin-3-yl)thiazol-4-yl)phenyl)pyrrolidin-2-one (95 mg, 81%) as a yellow oil. LCMS (ESI): mass calcd. for C₂₉H₂₆N₄O₄S₂, 558.67; m/z found 559.1 [M+H]⁺.

### Intermediate 19. (R)-3-(3-(4-Bromothiazol-2-yl)phenyl)-3-hydroxy-1-methylpyrrolidin-2-one

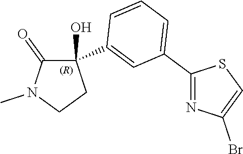

Step A. (R,S)-3-Hydroxy-1-methyl-3-(3-(thiazol-4-yl)phenyl)pyrrolidin-2-one. 4-Bromothiazole (7.68 g, 46.8 mmol) was added to the mixture of (R,S)-3-hydroxy-1-methyl-3-(3-(4,4,5,5-tetramethyl-1,3,2-dioxaborolan-2-yl)phenyl)pyrrolidin-2-one (Intermediate 4, 13.5 g, 42.6 mmol), K₃PO₄ (21.7 g, 102 mmol), and 1,4-dioxane/H₂O (5/1, 180 mL). The resultant mixture was sparged with Ar for 5 minutes and then treated with Pd(dtbpf)Cl₂ (2.77 g, 4.26 mmol). The resultant mixture was sparged with Ar for another 5 minutes and then heated at 90° C. for 2 h. The mixture was then concentrated to dryness and purified by FCC (eluent: petroleum ether:ethyl acetate=1:0 to 0:1) to yield (R,S)-3-hydroxy-1-methyl-3-(3-(thiazol-4-yl)phenyl)pyrrolidin-2-one (11 g, 92%) as a brown solid. LCMS (ESI): mass calcd for C₁₄H₁₄N₂O₂S, 274.08; m/z found, 275.1 [M+H]⁺.

Step B. (R,S)-3-(3-(2-Bromothiazol-4-yl)phenyl)-3-hydroxy-1-methylpyrrolidin-2-one. A solution of (R,S)-3-hydroxy-1-methyl-3-(3-(thiazol-4-yl)phenyl)pyrrolidin-2-one (11 g, 40 mmol) and THF (120 mL) was added dropwise to a solution of LDA (70.2 mL, 2.0 M in THF, 140 mmol) that had been cooled to −72° C. The resultant mixture was stirred at −72° C. for 30 minutes, then treated with Br₂ (3.70 mL, 72.2 mmol), and stirred at −72° C. for another 30 minutes. The mixture was then quenched with water (100 mL) and extracted with ethyl acetate (100 mL×3). The combined organic solvent extracts were washed with brine (300 mL), dried over anhydrous Na₂SO₄, filtered, and concentrated to yield the product which was purified by FCC (eluent: petroleum ether ethyl acetate=1:0 to 1:4) to yield (R,S)-3-(3-(2-bromothiazol-4-yl)phenyl)-3-hydroxy-1-methylpyrrolidin-2-one (5 g, 27%) as a brown solid. LCMS (ESI): mass calcd for C₁₄H₁₃BrN₂O₂S, 351.99: m/z found, 352.9 [M+H]⁺.

Step C. (R)-3-(3-(4-Bromothiazol-2-yl)phenyl)-3-hydroxy-1-methylpyrrolidin-2-one. The (R) and (S) enantiomers of (R,S)-3-(3-(2-Bromothiazol-4-yl)phenyl)-3-hydroxy-1-methylpyrrolidin-2-one were separated by preparative SFC using an SFC column, such as a DAICEL CHIRALPAK IC 250 mm×50 mm×10 μm column (isocratic elution: MeOH (containing 0.1% of 25% aqueous NH₃): supercritical CO₂, 45%: 55% to yield a first eluting enantiomer, (S)-3-(3-(4-Bromothiazol-2-yl)phenyl)-3-hydroxy-1-methylpyrrolidin-2-one and a second eluting enantiomer, (R)-3-(3-(4-Bromothiazol-2-yl)phenyl)-3-hydroxy-1-methylpyrrolidin-2-one. The fractions containing (R)-3-(3-(4-Bromothiazol-2-yl)phenyl)-3-hydroxy-1-methylpyrrolidin-2-one were collected and concentrated under reduced pressure. This product was suspended in water (10 mL) and the mixture was frozen by insertion into a −78° C. bath. The frozen mixture was then lyophilized to yield (R)-3-(3-(4-bromothiazol-2-yl)phenyl)-3-hydroxy-1-methylpyrrolidin-2-one (4.63 g, 41°%) as a gray solid. SFC Rₜ=2.729 min. LCMS (ESI): mass calcd for C₁₄H₁₃BrN₂O₂S, 351.99; m/z found, 353.0 [M+H]⁺. ¹H NMR (400 MHz, DMSO-d₆) δ 8.14 (s, 1H), 7.94 (s, 1H), 7.80 (d, J=7.8 Hz, 1H), 7.45-7.38 (m, 1H), 7.37-7.30 (m, 1H), 6.11 (s, 1H), 3.49-3.39 (m, 2H), 2.85 (s, 3H), 2.40-2.20 (m, 2H).

### Intermediate 20. (R)-3-Hydroxy-1-methyl-3-(3-(4-(1-(phenylsulfonyl)-1H-pyrrolo[2,3-b]pyridin-3-yl)thiazol-2-yl)phenyl)pyrrolidin-2-one

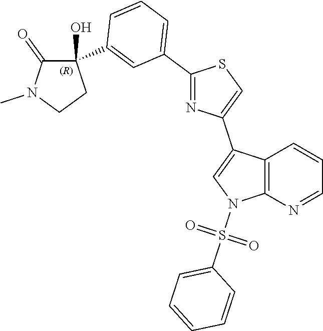

To a microwave vial containing (R)-3-hydroxy-1-methyl-3-(3-(4,4,5,5-tetramethyl-1,3,2-dioxaborolan-2-yl)phenyl)pyrrolidin-2-one (Intermediate 16, 250 mg, 0.63 mmol), 1-(phenylsulfonyl)-3-(4,4,5,5-tetramethyl-1,3,2-dioxaborolan-2-yl)-1H-pyrrolo[2,3-b]pyridine (319 mg, 0.830 mmol), and K₃PO₄ (308 mg, 2.26 mmol) was added 1,4-dioxane (6 mL), and H₂O (1.5 mL). The mixture was sparged with Ar for 5 minutes and then treated with Pd(dtbpf)Cl₂ (49 mg, 0.075 mmol). The resultant mixture was sparged with Ar for another 5 minutes and then subjected to microwave irradiation at 90° C. for 1 h. The reaction mixture was cooled to room temperature, concentrated to dryness under reduced pressure, and purified by FCC (eluent: petroleum ether:ethyl acetate=1:0 to 0:1) to yield (R)-3-hydroxy-1-methyl-3-(3-(4-(1-(phenylsulfonyl)-1H-pyrrolo[2,3-b]pyridin-3-yl)thiazol-2-yl)phenyl)pyrrolidin-2-one (220 mg, 65%) as a yellow solid. LCMS (ESI): mass calcd. for C₂₇H₂₂N₄O₄S₂, 530.62; m/z found 531.5[M+H]⁺.

### Intermediate 21. (R)-3-(3-(5-Bromothiazol-2-yl)phenyl)-3-hydroxy-1-methylpyrrolidin-2-one

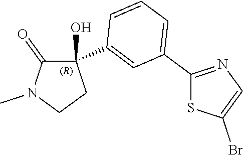

To a microwave tube containing (R)-3-hydroxy-1-methyl-3-(3-(4,4,5,5-tetramethyl-1,3,2-dioxaborolan-2-yl)phenyl)pyrrolidin-2-one (Intermediate 16, 100 mg, 0.32 mmol), 2,5-dibromothiazole (115 mg, 0.473 mmol), and Cs₂CO₃ (308 mg, 0.945 mmol) were added 1,4-dioxane (3 mL) and H₂O (0.6 mL). The resultant mixture was sparged with Ar for 5 minutes and then treated with Pd(dppf)Cl₂ (23 mg, 0.031 mmol). This mixture was then subjected to microwave irradiation at 110° C. for 1 h, was combined with another batch of the same material and then filtered through a pad of diatomaceous earth. The pad was further rinsed with ethyl acetate (20 mL). The filtrate was concentrated to dryness under reduced pressure and purified by FCC (petroleum ether:ethyl acetate=20:1 to 3:1) to yield (R)-3-(3-(5-bromothiazol-2-yl)phenyl)-3-hydroxy-1-methylpyrrolidin-2-one (60 mg) as a brown solid. ¹H NMR (400 MHz, CDCl₃) δ 7.93 (s, 1H), 7.78-7.75 (m, 1H), 7.74 (s, 1H), 7.44-7.42 (m, 2H), 3.48 (dd, J=8.6, 3.7 Hz, 1H), 3.42 (d, J=6.8 Hz, 1H), 3.04 (s, 3H), 2.54-2.49 (m, 1H), 2.47-2.45 (m, 1H), 2.05 (s, 1H).

### Intermediate 22. (R)-3-Hydroxy-1-methyl-3-(3-(5-(1-(phenylsulfonyl)-1H-pyrrolo[2,3-b]pyridin-3-yl)thiazol-2-yl)phenyl)pyrrolidin-2-one

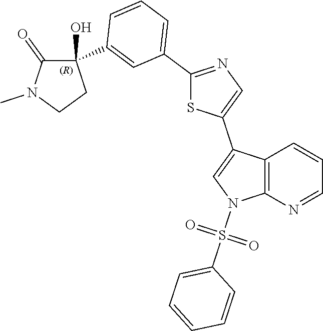

To a microwave tube contain ng (R)-3-(3-(5-bromothiazol-2-yl)phenyl)-3-hydroxy-1-methylpyrrolidin-2-one (Intermediate 21, 80 mg, 0.23 mmol), 1-(phenylsulfonyl)-3-(4,4,5,5-tetramethyl-1,3,2-dioxaborolan-2-yl)-1H-pyrrolo[2,3-b]pyridine (131 mg, 0.341 mmol), and Cs₂CO₃ (221 mg, 0.678 mmol) were added 1,4-dioxane (4 mL) and H₂O (0.8 mL). The resultant mixture was sparged with Ar for 5 minutes and then treated with Pd(dppf)Cl₂ (17 mg 0.023 mmol). This mixture was subjected to microwave irradiation at 110° C. for 1 h. The reaction mixture was then cooled to room temperature and the suspension was filtered through a pad of diatomaceous earth. The pad was rinsed with ethyl acetate (30 mL). The filtrate was concentrated to dryness under reduced pressure and purified by FCC (eluent: petroleum ether ethyl acetate containing 10% methanol)=20:1 to 0:1) to yield (R)-3-hydroxy-1-methyl-3-(3-(5-(1-(phenylsulfonyl)-1H-pyrrolo[2,3-b]pyridin-3-yl)thiazol-2-yl)phenyl)pyrrolidin-2-one (100 mg, 41%) as a brown solid. LCMS (ESI): mass calcd. for C₂₇H₂₂N₄O₄S₂, 530.62; m/z found 531.1 [M+H]⁺.

### Intermediate 23. (R)-7-(3-(2-(1-(Phenylsulfonyl)-1H-pyrrolo[2,3-b]pyridin-3-yl)thiazol-4-yl)phenyl)-6,7-dihydro-5H-pyrrolo[1,2-a]imidazol-7-ol

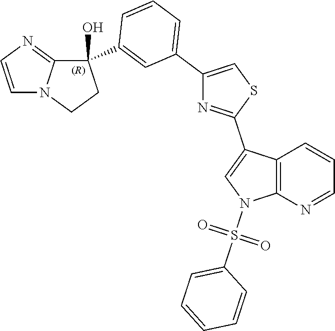

To a microwave tube containing 4-bromo-2-(1-(phenylsulfonyl)-1H-pyrrolo[2,3-b]pyridin-3-yl)thiazole (Intermediate 2, 200 mg, 0.48 mmol), (R)-7-(3-(4,4,5,5-tetramethyl-1,3,2-dioxaborolan-2-yl)phenyl)-6,7-dihydro-5H-pyrrolo[1,2-a]imidazol-7-ol, (Intermediate 13, 184 mg, 0.564 mmol), and K₃PO₄ (303 mg, 1.43 mmol) was added 1,4-dioxane (12 mL) and H₂O (3 mL). The mixture was sparged with Ar for 5 minutes and then treated with Pd(dtbpf)Cl₂ (31 mg, 0.048 mmol). The mixture was sparged with Ar for another 5 minutes and subjected to microwave irradiation at 80° C. for 1 h. The mixture was cooled to room temperature and filtered through a pad of diatomaceous earth which was further rinsed with ethyl acetate (30 mL). The combined organic solvent extracts were concentrated to dryness under reduced pressure and purified by FCC (initial eluent: petroleum ether:ethyl acetate=1:0 to 0:1, then second eluent: dichloromethane:methanol=1:0 to 10:1) to yield (R)-7-(3-(2-(1-(phenylsulfonyl)-1H-pyrrolo[2,3-b]pyridin-3-yl)thiazol-4-yl)phenyl)-6,7-dihydro-5H-pyrrolo[1,2-a]imidazol-7-ol (200 mg) as a brown solid. LCMS(ESI): mass calcd. for C₂₈H₂₁N₅O₃S₂, 539.11; m/z found, 540.1 [M+H]⁺.

### Intermediate 24. (R)-7-(3-(4-Bromothiazol-2-yl)phenyl)-6,7-dihydro-5H-pyrrolo[1,2-a]imidazol-7-ol

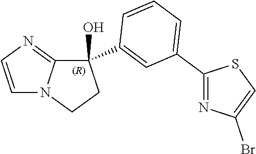

To a microwave tube containing (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 600 mg, 1.8 mmol), 2,4-dibromothiazole (782 mg, 3.22 mmol), Cs₂CO₃ (1.75 g, 5.37 mmol), was added 1,4-dioxane (12 mL) and H₂O (3 mL). The mixture was sparged with Ar for 5 minutes and then treated with Pd(dppf)Cl₂ (157 mg, 0.215 mmol). The mixture was sparged with Ar for another 5 minutes and then subjected to microwave irradiation at 80° C. for 1 h. The reaction mixture was cooled to room temperature and the suspension was filtered through a pad of diatomaceous earth and the pad washed with ethyl acetate (50 mL). The filtrate was concentrated to dryness under reduced pressure and purified by FCC (eluent: petroleum ether:ethyl acetate=1:0 to 0:1) to yield (R)-7-(3-(4-bromothiazol-2-yl)phenyl)-6,7-dihydro-5H-pyrrolo[1,2-a]imidazol-7-ol (400 mg, 48%) as a brown oil. LCMS (ESI): mass calcd. for C₁₅H₁₂BrN₃OS, 360.99; m/z found, 362.0 [M+H]⁺. ¹H NMR (400 MHz, DMSO-d₆) δ 8.06 (s, 1H), 7.87 (s, 1H), 7.84-7.75 (m, 1H), 7.51-7.44 (m, 2H), 7.21-7.17 (m, 1H), 7.02 (s, 1H), 6.29 (s, 1H), 4.15-4.03 (m, 2H), 2.86-2.68 (m, 2H).

### Intermediate 25. (R)-7-(3-(4-(5-Tosyl-5H-pyrrolo[2,3-b]pyrazin-7-yl)thiazol-2-yl)phenyl)-6,7-dihydro-5H-pyrrolo[1,2-a]imidazol-7-ol

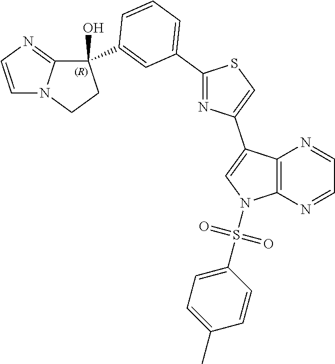

To a microwave tube containing (R)-7-(3-(4-bromothiazol-2-yl)phenyl)-6,7-dihydro-5H-pyrrolo[1,2-a]imidazol-7-ol (Intermediate 24, 200 mg, 0.552 mmol), 7 (4,4,5,5-tetramethyl-1,3,2-dioxaborolan-2-yl)-5-tosyl-5H-pyrrolo[2,3-b]pyrazine (Intermediate 9, 280 mg, 0.701 mmol), and K₃PO₄ (349 mg, 1.64 mmol) was added 1,4-dioxane (12 mL), and H₂O (3 mL). The mixture was sparged with Ar for 5 minutes and then treated with Pd(dtbpf)Cl₂ (36 mg, 0.055 mmol). The mixture was sparged with Ar for another 5 minutes and then heated at 100° C. in microwave for 1 h. The reaction mixture was cooled to room temperature, filtered through a pad of diatomaceous earth. The filtrate was concentrated to dryness and purified by FCC (eluent: petroleum ether:ethyl acetate=1:0 to 0:1) to yield (R)-7-(3-(4-(5-tosyl-5H-pyrrolo[2,3-b]pyrazin-7-yl)thiazol-2-yl)phenyl)-6,7-dihydro-5H-pyrrolo[1,2-a]imidazol-7-ol (250 mg, 80%) as a brown oil. LCMS (ESI): mass calcd. for C₂₈H₂₂N₆O₃S₂, 554.12; m/z found, 555.1 [M+H]⁺.

### Intermediate 26. (R)-7-(3-Bromophenyl)-7H-pyrrolo[1,2-a]imidazol-7-ol

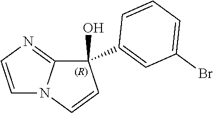

Step A. 6-(Phenylselanyl)-5,6-dihydro-7H-pyrrolo[1,2-a]imidazol-7-one. NaHMDS (8.0 mL, 1 M in tetrahydrofuran, 8.0 mmol) was added drop-wise to a solution of 5H-pyrrolo[1,2-a]imidazol-7(6H)-one (1.0 g, 8.2 mmol) and tetrahydrofuran (20 mL) that had been cooled to −70° C., under a N₂ atmosphere. The resultant mixture was stirred at −70° C. for 0.5 h, to which phenylselenyl chloride (1.42 g, 7.41 mmol) in tetrahydrofuran (10 mL) was added. The mixture was stirred at −70° C. for 20 min and then quenched with aqueous saturated NH₄Cl solution (50 mL). The reaction mixture was extracted with ethyl acetate (50 mL×3) and the combined organic solvent extracts were dried over anhydrous Na₂SO₄, filtered, and concentrated to dryness under reduced pressure. The product was purified by FCC (eluent: petroleum ether:ethyl acetate=1:0 to 1:9) to yield 6-(phenylselanyl)-5,6-dihydro-7H-pyrrolo[1,2-a]imidazol-7-one (800 mg, 34%) as a red oil. LCMS (ESI): mass calcd. for C₁₂H₁₀N₂Ose, 278.00; m/z found, 278.9 [M+H]⁺.

Step B. (R,S)-7-(3-bromophenyl)-6-(phenylselanyl)-6,7-dihydro-5H-pyrrolo[1,2-a]imidazol-7-ol. n-BuLi (3.2 mL, 2.5 M in tetrahydrofuran, 8 mmol) was added drop-wise to a cooled −60° C. solution of 1,3-dibromobenzene (3.2 g, 14 mmol) and anhydrous tetrahydrofuran (20 mL). The resultant mixture was stirred at −60° C. for 1.5 h. Then a solution of 6-(phenylselenyl)-5H-pyrrolo[1,2-a]imidazol-7(6H)-one (800 mg, 2.89 mmol) and anhydrous tetrahydrofuran (10 mL) was added drop-wise to above solution. The resultant mixture was stirred for 2 h with gradual warming to room temperature and then quenched with aqueous saturated NH₄Cl solution (50 mL). The reaction mixture was extracted with ethyl acetate (50 mL×3) and the combined organic solvent extracts were dried over anhydrous Na₂SO₄, filtered, and concentrated to dryness under reduced pressure to yield the product which was purified by FCC (eluent: petroleum ether:ethyl acetate=1:0 to 1:1) which yielded (R,S)-7-(3-bromophenyl)-6-(phenylselanyl)-6,7-dihydro-5H-pyrrolo[1,2-a]imidazol-7-ol as a yellow oil. LCMS (ESI): mass calcd. for C₁₈H₁₅BrN₂Ose, 433.95; m/z found, 434.9 [M+H]⁺.

Step C. (R,S)-7-(3-Bromophenyl)-7H-pyrrolo[1,2-a]imidazol-7-ol. To a flask containing (R,S)-7-(3-bromophenyl)-6-(phenylselanyl)-6,7-dihydro-5H-pyrrolo[1,2-a]imidazol-7-ol (70 mg), Et₂NH (14.0 mg, 0.19 mmol) and tetrahydrofuran (1 mL) was added H₂O₂ (32 mg, 30% in H₂O, 0.28 mmol) at 0° C. The resultant mixture was stirred for 2 h with gradual warming to room temperature and then quenched with saturated Na₂SO₃ solution (15 mL). The reaction mixture was extracted with ethyl acetate (20 mL×3) and the combined organic solvent extracts were washed with brine (10 mL), dried over anhydrous Na₂SO₄, filtered, and concentrated to dryness. The product was purified by preparative HPLC using an HPLC column, such as a Welch Xtimate C18 100 mm×40 mm×3 μm column (eluent: 5% to 35%, CH₃CN and H₂O (with 0.225% HCOOH)) to yield the product. This material was combined with another batch and suspended in water (10 mL) and the mixture frozen by insertion into a −78° C. bath. The frozen mixture was then lyophilized to yield (R,S)-7-(3-bromophenyl)-7H-pyrrolo[1,2-a]imidazol-7-ol (150 mg). LCMS (ESI): mass calcd. for C₁₂H₉BrN₂O, 277.11; m/z found, 278.9 [M+H]⁺.

Step D. (R)-7-(3-Bromophenyl)-7H-pyrrolo[1,2-a]imidazol-7-ol. The enantiomers of (R,S)-7-(3-Bromophenyl)-7H-pyrrolo[1,2-a]imidazol-7-ol (150 mg, 0.541 mmol) were separated by SFC using an SFC column, such as a DAICEL CHIRALPAK AD 250 mm×30 mm, 10 μm (isocratic elution: i-PrOH (containing 0.1% of 25% aqueous NH₃): supercritical CO₂, 40%: 60%). The first eluting enantiomer, (R)-7-(3-bromophenyl)-7H-pyrrolo[1,2-a]imidazol-7-ol, was obtained as a colorless oil (70 mg, 45%), SFC Rₜ=1.334 min. The second eluting enantiomer, (S)-7-(3-bromophenyl)-7H-pyrrolo[1,2-a]imidazol-7-ol (Intermediate 27) was also obtained.

### Intermediate 27. (S)-7-(3-Bromophenyl)-7H-pyrrolo[1,2-a]imidazol-7-ol

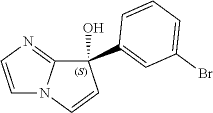

(S)-7-(3-bromophenyl)-7H-pyrrolo[1,2-a]imidazol-7-ol (70 mg, 46%) was obtained from the chiral separation described in Intermediate 26, Step D, as a colorless oil. SFC Rₜ=1.703 min.

### Intermediate 28. (R)-7-(3-(4,4,5,5-Tetramethyl-1,3,2-dioxaborolan-2-yl)phenyl)-7H-pyrrolo[1,2-a]imidazol-7-ol

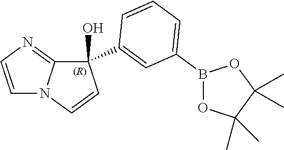

To a microwave tube containing (R)-7-(3-Bromophenyl)-7H-pyrrolo[1,2-a]imidazol-7-ol (Intermediate 26, 70 mg, 0.25 mmol), 4,4,4′,4′,5,5,5′,5′-octamethyl-2,2′-bi(1,3,2-dioxaborolane) (128 mg, 0.504 mmol), and KOAc (74 mg, 0.75 mmol) was added 1,4-dioxane (5 mL). The mixture was sparged with N₂ for 5 minutes and then treated with Pd(dppf)Cl₂ (18 mg, 0.03 mmol). The resultant mixture was subjected to microwave irradiation at 90° C. for 1 h. The reaction mixture was cooled to room temperature and the suspension was concentrated to dryness under reduced pressure. The product was purified by FCC (eluent: petroleum ether:ethyl acetate=10:1 to 1:2) to yield (R)-7-(3 (4,4,5,5-tetramethyl-1,3,2-dioxaborolan-2-yl)phenyl)-7H-pyrrolo[1,2-a]imidazol-7-ol (65 mg, 70%) as a brown oil. LCMS (ESI): mass calcd. for C₁₈H₂₁BN₂O₃ 324.16; m/z found, 325.2 [M+H]⁺.

### Intermediate 29. (R)-7-(3-(2-(5-Tosyl-5H-pyrrolo[2,3-b]pyrazin-7-yl)thiazol-4-yl)phenyl)-7H-pyrrolo[1,2-a]imidazol-7-ol

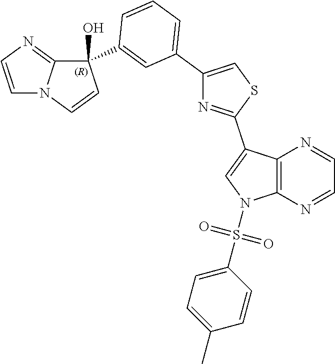

To a microwave tube containing 4-bromo-2-(5-tosyl-5H-pyrrolo[2,3-b]pyrazin-7-yl)thiazole, (Intermediate 10, 87 mg, 0.20 mmol), (R)-7-(3-(4,4,5,5-tetramethyl-1,3,2-dioxaborolan-2-yl)phenyl)-7H-pyrrolo[1,2-a]imidazol-7-ol (Intermediate 28, 65 mg, 0.20 mmol), and K₃PO₄ (128 mg, 0.603 mmol) was added 1,4-dioxane (5 mL) and H₂O (1 mL). The mixture was sparged with Ar for 5 minutes and then treated with Pd(dtbpf)Cl₂ (13 mg, 0.020 mmol). The mixture was sparged with Ar for another 5 minutes and the resultant mixture was subjected to microwave irradiation at 80° C. for 1 h. The reaction mixture was cooled to room temperature and concentrated to dryness under reduced pressure and the residue was purified by FCC (eluent: petroleum ether:ethyl acetate=10:1 to 0:1) yield (R)-7-(3-(2-(5-tosyl-5H-pyrrolo[2,3-b]pyrazin-7-yl)thiazol-4-yl)phenyl)-7H-pyrrolo[1,2-a]imidazol-7-ol (60 mg, 54%) as a brown solid. LCMS (ESI): mass calcd. for C₂₈H₂₀NeO₃S₂, 552.10; m/z found, 553.1 [M+H]⁺.

### Intermediate 30. (S)-7-(3-(4,4,5,5-Tetramethyl-1,3,2-dioxaborolan-2-yl)phenyl)-7H-pyrrolo[1,2-a]imidazol-7-ol

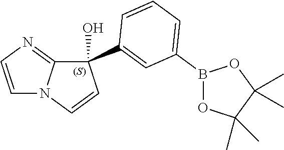

(S)-7-(3-(4, 4, 5,5-Tetramethyl-1,3,2-dioxaborolan-2-yl)phenyl)-7H-pyrrolo[1,2-a]imidazol-7-ol was prepared in a manner analogous to Intermediate 28, using (S)-7-(3-bromophenyl)-7H-pyrrolo[1,2-a]imidazol-7-ol, (Intermediate 27) in place of (R)-7-(3-bromophenyl)-7H-pyrrolo[1,2-a]imidazol-7-ol, (Intermediate 26). LCMS (ESI): mass calcd. for C₁₈H₂₁BN₂O₃, 324.16; m/z found, 325.2 [M+H]⁺.

### Intermediate 31. (S)-7-(3-(2-(5-Tosyl-5H-pyrrolo[2,3-b]pyrazin-7-yl)thiazol-4-yl)phenyl)-7H-pyrrolo[1,2-a]imidazol-7-ol

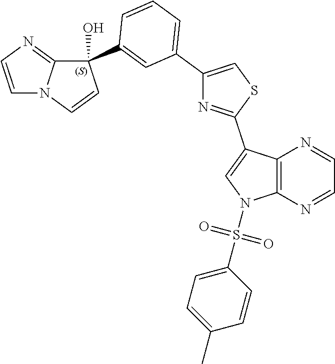

(S)-7-(3-(2-(5-Tosyl-5H-pyrrolo[2,3-b]pyrazin-7-yl)thiazol-4-yl)phenyl)-7H-pyrrolo[1,2-a]imidazol-7-ol was prepared in a manner analogous to the preparation of Intermediate 29, using (S)-7-(3-(4,4,5,5-Tetramethyl-1,3,2-dioxaborolan-2-yl)phenyl)-7H-pyrrolo[1,2-a]imidazol-7-ol (Intermediate 30) in place of (R)-7-(3-(4,4,5,5-tetramethyl-1,3,2-dioxaborolan-2-yl)phenyl)-7H-pyrrolo[1,2-a]imidazol-7-ol (Intermediate 28). LCMS (ESI): mass calcd. for C₂₈H₂₀N₆O₃S₂, 552.10; m/z found, 553.2 [M+H]⁺.

### Intermediate 32. (R)-7-(3-(4-(1-(phenylsulfonyl)-1H-pyrrolo[2,3-b]pyridin-3-yl)thiazol-2-yl)phenyl)-6,7-dihydro-5H-pyrrolo[1,2-a]imidazol-7-ol

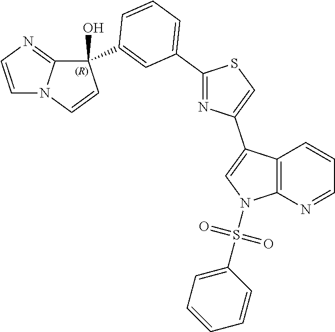

To a microwave tube containing (R)-7-(3-(4-Bromothiazol-2-yl)phenyl)-6,7-dihydro-5H-pyrrolo[1,2-a]imidazol-7-ol (Intermediate 24, 200 mg, 0.5 mmol), 1-(phenylsulfonyl)-3-(4,4,5,5-tetramethyl-1,3,2-dioxaborolan-2-yl)-1H-pyrrolo[2,3-b]pyridine (270 mg, 0.703 mmol), and K₃PO₄ (349 mg, 1.64 mmol) was added 1,4-dioxane (12 mL) and H₂O (3 mL). The mixture was sparged with Ar for 5 minutes and then treated with Pd(dtbpf)Cl₂ (36 mg, 0.055 mmol). The mixture was sparged with Ar for another 5 minutes and then heated at 85° C. in microwave for 1 h. The reaction mixture was cooled to room temperature, concentrated to dryness and purified by FCC (eluent: petroleum ether:ethyl acetate=1:0 to 0:1) to yield (R)-7-(3-(4-(1-(phenylsulfonyl)-1H-pyrrolo[2,3-b]pyridin-3-yl)thiazol-2-yl)phenyl)-6,7-dihydro-5H-pyrrolo[1,2-a]imidazol-7-ol (150 mg, 49%) as a brown oil. LCMS(ESI): R_{T}=2.39 min, mass calcd. for C₂₈H₂₁N₅O₃S₂, 539.11; m/z found, 540.3 [M+H]⁺.

### Intermediate 33. Furo[3,2-b]pyridin-3(2H)-one

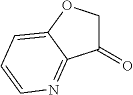

Step A. Ethyl-3-hydroxypicolinate. To a flask was added 3-hydroxypicolinic acid (30.0 g, 216 mmol), EtOH (360 ml, 6.17 mol), H₂SO₄ (10.0 mL), and toluene (100 mL). The resultant mixture was heated at 78° C. for 48 h. The reaction mixture was allowed to cool to room temperature and the resulting mixture was quenched with aqueous NaOH (0.92 mol in 150 mL of water) and extracted with ethyl acetate (200 mL×3). The combined organic solvent extracts were dried over anhydrous Na₂SO₄, filtered, and concentrated, and the residue was purified by FCC (eluent: petroleum ether:ethyl acetate=10:1 to 5:1) to yield ethyl-3-hydroxypicolinate (23.8 g, 63%) as a colorless oil. LCMS (ESI): mass calcd. for C₈H₉NO₃, 167.16; m/z found, 168.1 [M+H]⁺.

Step B. Ethyl 3-(2-ethoxy-2-oxoethoxy)picolinate. Ethyl 2-bromoacetate (18.1 mL, 163 mmol) was added to a solution of ethyl 3-hydroxypicolinate (23.8 g, 142 mmol), K₂CO₃ (59.0 g, 427 mmol), and acetone (250 mL). The resultant mixture was heated at 60° C. for 15 h and then allowed to cool to room temperature. The reaction mixture was extracted with EtOAc and combined organic solvent extracts were dried, filtered, and concentrated. The residue was purified by FCC (eluent: petroleum ether ethyl acetate=10:1 to 4:1) to yield ethyl 3-(2-ethoxy-2-oxoethoxy)picolinate (22 g, 61%) as a yellow oil. ¹H NMR (400 MHz, DMSO-d₆) δ 8.23-8.16 (m, 1H), 7.58-7.48 (m, 2H), 4.92 (s, 2H), 4.35-4.29 (m, 2H), 4.16 (q, J=7.1 Hz, 2H), 1.30 (t, J=7.1 Hz, 3H), 1.20 (t, J=7.1 Hz, 3H).

Step C. Ethyl 3-hydroxyfuro[3,2-b]pyridine-2-carboxylate. Ethyl 3-(2-ethoxy-2-oxoethoxy)picolinate (22 g, 87 mmol) was added to a solution of sodium ethanolate (71.2 mL, 21% in EtOH, 191 mmol) and toluene (150 mL). The resultant mixture was heated at 111° C. for 18 h and then allowed to cool to room temperature. The pH of the mixture was adjusted to pH=7-8 with 12 M HCl and it was then extracted with ethyl acetate (150 mL×3). The combined organic solvent extracts were dried over anhydrous Na₂SO₄, filtered, and concentrated. The residue was purified by FCC (eluent: petroleum ether:ethyl acetate=1:0 to 1:2) to yield ethyl 3-hydroxyfuro[3,2-b]pyridine-2-carboxylate (8.6 g, 48%) as a white solid. ¹H NMR (400 MHz, DMSO-d₆) S 11.73-10.90 (m, 1H), 8.63 (dd, J=4.4, 1.1 Hz, 1H), 8.07 (dd, J=8.4, 1.1 Hz, 1H), 7.55 (dd, J=8.6, 4.6 Hz, 1H), 4.32 (q, J=7.2 Hz, 2H), 1.31 (t, J=7.2 Hz, 3H).

Step D. Furo[3,2-b]pyridin-3(2H)-one. To a flask containing ethyl 3-hydroxyfuro[3,2-b]pyridine-2-carboxylate (3.00 g, 14.5 mmol) was added HCl (3 M, 120 mL). The resultant mixture was heated at 100° C. for 5 h and then allowed to cool to room temperature. The reaction mixture was adjusted to pH=6-7 by the addition of 1M HCl and then extracted with ethyl acetate (100 mL×3). The combined organic solvent extracts were concentrated to yield furo[3,2-b]pyridin-3(2H)-one (1.8 g) as brown oil, which was used next step without further purification. ¹H NMR (400 MHz, DMSO-d₆) S 8.53-8.49 (m, 1H), 7.82-7.79 (m, 1H), 7.69-7.64 (m, 1H), 4.89 (s, 2H).

### Intermediate 34. (S)-3-(3-Bromophenyl)-2,3-dihydrofuro[3,2-b]pyridin-3-ol

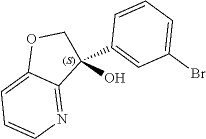

Step A. (R,S)-3-(3-bromophenyl)-2,3-dihydrofuro[3,2-b]pyridin-3-ol. A solution of 1,3-dibromobenzene (6.9 g, 29 mmol) and tetrahydrofuran (40 mL) was cooled to −70° C. and n-BuLi (11.7 mL, 2.5 M in hexane, 29.3 mmol) was added. The resultant mixture was stirred at −70° C. for 30 min and then treated with a solution of furo[3,2-b]pyridin-3(2H)-one (1.8 g) and tetrahydrofuran (2 mL). The mixture was gradually allowed to warm to room temperature and then stirred for 16 h. The reaction mixture was quenched with aqueous saturated NH₄Cl solution (80 mL) and extracted with ethyl acetate (80 mL×3). The combined organic solvent extracts were dried over anhydrous Na₂SO₄, filtered, and concentrated. The residue was initially purified by FCC (eluent: petroleum ether ethyl acetate=10:1 to 2:1). The residue was further purified by HPLC using an HPLC column, such as an Xtimate C18, 150×40 mm×10 μm column (eluent: 25% to 55%, CH₃CN and H₂O with (0.04% NH₄OH+10 mM NH₄HCO₃)). The product was suspended in water (10 mL) and the mixture was frozen by insertion into a −78° C. bath. The frozen mixture was then lyophilized to yield (R,S)-3-(3-bromophenyl)-2,3-dihydrofuro[3,2-b]pyridin-3-ol (190 mg, 5%) as a brown oil. LCMS (ESI): mass calcd. for C₁₃H₁₀BrNO₂, 290.99; m/z found, 292.1 [M+H]⁺.

Step B. (S)-3-(3-bromophenyl)-2,3-dihydrofuro[3,2-b]pyridin-3-ol. The enantiomers of (R,S)-3-(3-bromophenyl)-2,3-dihydrofuro[3,2-b]pyridin-3-ol (260 mg, 0.890 mmol) were separated by SFC using an SFC column, such as a Phenomenex-Cellulose-2 250 mm×30 mm, 10 μm column (isocratic elution: EtOH (containing 0.1% of 25% aqueous NH₃): supercritical CO₂, 25%: 75%). The fractions were collected and concentrated under reduced pressure to yield the first eluting product, (S)-3-(3-bromophenyl)-2,3-dihydrofuro[3,2-b]pyridin-3-ol (110 mg, 41%) as a clear oil, and the second eluting enantiomer, (R)-3-(3-bromophenyl)-2,3-dihydrofuro[3,2-b]pyridin-3-ol (Intermediate 35). Data for (S)-3-(3-bromophenyl)-2,3-dihydrofuro[3,2-b]pyridin-3-ol: SFC Rₜ=2.853 min, LCMS (ESI): mass calcd. for C₁₃H₁₀BrNO₂, 290.99; m/z found, 292.1 [M+H]⁺.

### Intermediate 35. (R)-3-(3-bromophenyl)-2,3-dihydrofuro[3,2-b]pyridin-3-ol

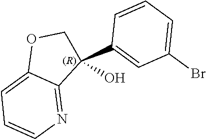

(R)-3-(3-bromophenyl)-2,3-dihydrofuro[3,2-b]pyridin-3-ol was obtained from the chiral separation described for Intermediate 34 (110 mg, 41%) as a clear oil. SFC Rₜ=3.101 min, LCMS (ESI): mass calcd. for C₁₃H₁₀BrNO₂, 290.99; m/z found, 292.0 [M+H]⁺.

### Intermediate 36. (R)-3-(3-(4,4,5,5-Tetramethyl-1,3,2-dioxaborolan-2-yl)phenyl)-2,3-dihydrofuro[3,2-b]pyridin-3-ol

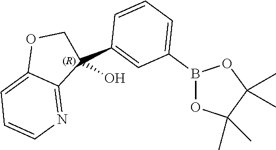

To a microwave tube containing 4,4,4′,4′,5,5,5′,5′-octamethyl-2,2′-bi(1,3,2-dioxaborolane), (245 mg, 0.965 mmol), KOAc (111 mg, 1.13 mmol), and 1,4-dioxane (10 mL) was added (R)-3-(3-bromophenyl)-2,3-dihydrofuro[3,2-b]pyridin-3-ol (Intermediate 35,110 mg, 0.38 mmol). The mixture was sparged with Ar for 5 minutes and then treated with Pd(dppf)Cl₂ (28 mg, 0.038 mmol). The mixture was sparged with Ar for another 5 minutes and then subjected to microwave irradiation at 85° C. for 1 h. The reaction mixture was cooled to room temperature, concentrated to dryness, and purified by FCC (eluent: petroleum ether:ethyl acetate=1:0 to 0:1) to yield (R)-3-(3-(4,4,5,5-tetramethyl-1,3,2-dioxaborolan-2-yl)phenyl)-2,3-dihydrofuro[3,2-b]pyridin-3-ol (120 mg, 80%) as a brown oil. LCMS (ESI): mass calcd. for C₁₉H₂₂BNO₄, 339.16; m/z found, 340.1 [M+H]⁺.

### Intermediate 37. (R)-3-(3-(2-(1-(Phenylsulfonyl)-1H-pyrrolo[2,3-b]pyridin-3-yl)thiazol-4-yl)phenyl)-2,3-dihydrofuro[3,2-b]pyridin-3-ol

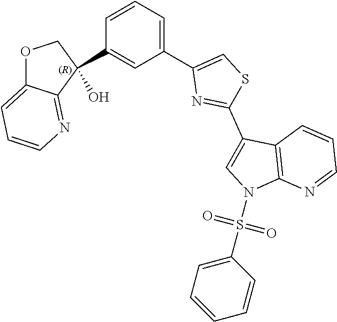

To a microwave tube containing 4-Bromo-2-(1-(phenylsulfonyl)-1H-pyrrolo[2,3-b]pyridin-3-yl)thiazole (Intermediate 2, 150 mg, 0.36 mmol), (R)-3-(3-(4,4,5,5-tetramethyl-1,3,2-dioxaborolan-2-yl)phenyl)-2,3-dihydrofuro[3,2-b]pyridin-3-ol (Intermediate 36, 120 mg, 0.35 mmol), and K₃PO₄ (225 mg, 1.06 mmol) was added 1,4-dioxane (12 mL) and H₂O (3 mL). The mixture was sparged with Ar for 5 minutes and then Pd(dtbpf)Cl₂ (23 mg, 0.035 mmol) was added. The mixture was sparged with Ar for another 5 minutes and then subjected to microwave irradiation at 80° C. for 1 h. The reaction mixture was then cooled to room temperature, concentrated under reduced pressure, and purified by FCC (eluent: petroleum ether:ethyl acetate=1:0 to 0:1) to yield (R)-3-(3-(2-(1-(phenylsulfonyl)-1H-pyrrolo[2,3-b]pyridin-3-yl)thiazol-4-yl)phenyl)-2,3-dihydrofuro[3,2-b]pyridin-3-ol (120 mg, 54%) as a yellow solid. LCMS (ESI): mass calcd. for C₂₉H₂₀N₄O₄S₂, 552.09; m/z found, 553.3 [M+H]⁺.

### Intermediate 38. (R)-3-(3-(2-(5-Tosyl-5H-pyrrolo[2,3-b]pyrazin-7-yl)thiazol-4-yl)phenyl)-2,3-dihydrofuro[3,2-b]pyridin-3-ol

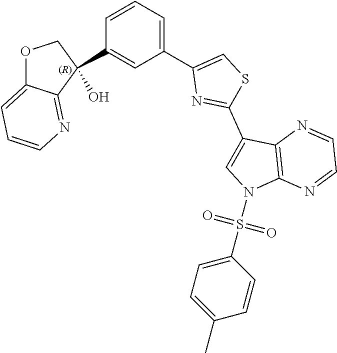

To a microwave tube containing 4-bromo-2-(5-tosyl-5H-pyrrolo[2,3-b]pyrazin-7-yl)thiazole (Intermediate 10, 120 mg, 0.28 mmol), (R)-3-(3-(4,4,5,5-tetramethyl-1,3,2-dioxaborolan-2-yl)phenyl)-2,3-dihydrofuro[3,2-b]pyridin-3-ol (Intermediate 36, 90 mg, 0.3 mmol), and K₃PO₄ (169 mg, 0.796 mmol) were added 1,4-dioxane (8 mL) and H₂O (2 mL). The mixture was sparged with Ar for 5 minutes and then Pd(dtbpf)Cl₂ (17 mg, 0.026 mmol) was added. The mixture was sparged with Ar for another 5 minutes and then subjected to microwave irradiation at 80° C. for 1 h. The reaction mixture was concentrated to dryness under reduced pressure and purified by FCC (eluent: petroleum ether:ethyl acetate=1:0 to 0:1) to yield (R)-3-(3-(2-(5-tosyl-5H-pyrrolo[2,3-b]pyrazin-7-yl)thiazol-4-yl)phenyl)-2,3-dihydrofuro[3,2-b]pyridin-3-ol (70 mg, 38%) as a white solid. LCMS (ESI): mass calcd. for C₂₉H₂₁N₅O₄S₂, 567.10; m/z found, 568.0 [M+H]⁺.

### Intermediate 39. (S)-7-(3-(4,4,5,5-Tetramethyl-1,3,2-dioxaborolan-2-yl)phenyl)-6,7-dihydro-5H-pyrrolo[1,2-a]imidazol-7-ol

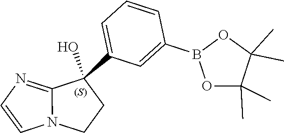

(S)-7-(3-(4,4,5,5-Tetramethyl-1,3,2-dioxaborolan-2-yl)phenyl)-6,7-dihydro-5H-pyrrolo[1,2-a]imidazol-7-ol was prepared in analogous manner to Intermediate 13 using (S)-7-(3-bromophenyl)-6,7-dihydro-5H-pyrrolo[1,2-a]imidazol-7-ol (Intermediate 12) instead of (R)-7-(3-bromophenyl)-6,7-dihydro-5H-pyrrolo[1,2-a]imidazol-7-ol (Intermediate 11).

### Intermediate 40. 7-(4,4,5,5-Tetramethyl-1,3,2-dioxaborolan-2-yl)-5-tosyl-5H-pyrrolo[2,3-b]pyrazine-6-d

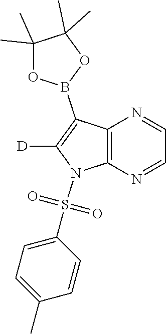 [SMILES: [2H]c1c(B2OC(C)(C)C(C)(C)O2)c2nccnc2n1S(=O)(=O)c1ccc(C)cc1]

Step A. 5-Tosyl-5H-pyrrolo[2,3-b]pyrazine-6-d. n-BuLi (4.4 mL, 2.5 M in hexane, 11 mmol) was added drop-wise to a solution of 5-tosyl-5H-pyrrolo[2,3-b]pyrazine (2.0 g, 7.3 mmol) and THE (20 mL) that had been cooled to −78° C. The resultant mixture was stirred at −78° C. for 1 h, and then quenched with D₂O (12.0 mL, 659 mmol). The mixture was stirred at room temperature for 30 minutes and extracted with ethyl acetate (15 mL×3). The combined organic solvent extracts were dried with anhydrous Na₂SO₄, filtered and concentrated to dryness under reduced pressure. The product was purified by FCC (eluent: petroleum ether:ethyl acetate=1:0 to 1:1) to yield 5-tosyl-5H-pyrrolo[2,3-b]pyrazine-6-d. (1.4 g, 67%) as a yellow solid. ¹H NMR (400 MHz, DMSO-d₆) δ 8.60 (d, J=2.6 Hz, 1H), 8.42 (d, J=2.4 Hz, 1H), 8.00 (d, J=8.4 Hz, 2H), 7.44 (d, J=7.9 Hz, 2H), 7.02 (s, 1H), 2.34 (s, 3H).

Step B. 7-Bromo-5-tosyl-5H-pyrrolo[2,3-b]pyrazine-6-d. To 5-tosyl-5H-pyrrolo[2,3-b]pyrazine-6-d (1.0 g, 3.6 mmol) in DMF (12 mL) that had been cooled to 0° C. was added N-bromosuccinimide (780 mg, 4.4 mmol). The resultant mixture was allowed to gradually warm room temperature and stirred for 16 h. The reaction mixture was quenched with saturated aqueous Na₂S₂O₃ solution and extracted with ethyl acetate (20 mL×4). The combined organic solvent extracts were washed with brine (50 mL), dried over anhydrous Na₂SO₄, filtered and concentrated to dryness under reduced pressure. The product was purified by FCC (petroleum ether:ethyl acetate=1:0 to 2:1) to yield 7-bromo-5-tosyl-5H-pyrrolo[2,3-b]pyrazine-6-d (1.4 g, 78%) as a yellow solid. LCMS (ESI): mass calcd. for C₁₃H₉BrDN₃O₂S, 353.2; m/z found, 354.9 [M+H]⁺.

Step C. 7-(4,4,5,5-tetramethyl-1,3,2-dioxaborolan-2-yl)-5-tosyl-5H-pyrrolo[2,3-b]pyrazine-6-d. To a flask containing potassium acetate (833 mg, 8.49 mmol), 7-bromo-5-tosyl-5H-pyrrolo[2,3-b]pyrazine-6-d (1.5 g, 5.9 mmol) was added 1,4-dioxane (13 mL). The mixture was purged with N₂ for three times and then Pd(dppf)Cl₂ (208 mg, 0.284 mmol) was added. The resultant mixture was purged with N₂ for another three times and heated at 100° C. for 3 h. The mixture was cooled to room temperature, filtered through a pad of diatomaceous earth and the filtrate was concentrated to dryness under reduced pressure. The product was purified by FCC (eluent: petroleum ether:ethyl acetate=1:0 to 1:1) to yield 7-(4,4,5,5-tetramethyl-1,3,2-dioxaborolan-2-yl)-5-tosyl-5H-pyrrolo[2,3-b]pyrazine-6-d (1.0 g, 53%) as a yellow solid. LCMS (ESI): mass calcd. for C₁₉H₂₁BDN₃O₄S, 400.15; m/z found, 318.9 [M−82+H]⁺.

### Intermediate 41. 4-Bromo-2-(5-tosyl-5H-pyrrolo[2,3-b]pyrazin-7-yl-6-d)thiazole

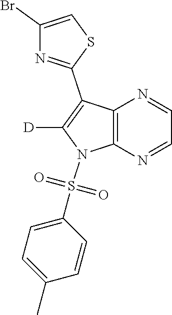 [SMILES: [2H]c1c(-c2nc(Br)cs2)c2nccnc2n1S(=O)(=O)c1ccc(C)cc1]

To a microwave tube containing cesium carbonate (1.09 g, 3.35 mmol), 7-(4,4,5,5-tetramethyl-1,3,2-dioxaborolan-2-yl)-5-tosyl-5H-pyrrolo[2,3-b]pyrazine-6-d (Intermediate 40, 1.1 g, 1.7 mmol), and 2,4-dibromothiazole (610 mg, 2.51 mmol) was added 1,4-dioxane (8 mL) and H₂O (2 mL). The mixture was purged with Ar for 5 minutes and then Pd(dppf)Cl₂ (133 mg, 0.182 mmol) was added. The resultant mixture was purged with Ar for another 5 minutes and subjected to microwave irradiation at 85° C. for 1 h. The reaction mixture was cooled to room temperature, diluted with water (15 mL) and the aqueous portion was extracted with ethyl acetate (20 mL×5). The combined organic solvent extracts were dried with anhydrous Na₂SO₄, filtered and concentrated to dryness. This material was purified by FCC (eluent: petroleum ether:ethyl acetate=1:0 to 2:1) to yield 4-bromo-2-(5-tosyl-5H-pyrrolo[2,3-b]pyrazin-7-yl-6-d)thiazole (200 mg, 27%) as a yellow solid. LCMS (ESI): mass calcd. for C₁₆H₁₀BrDN₄O₂S₂, 436.2; m/z found, 437.9 [M+H]⁺. ¹H NMR (400 MHz, DMSO-d₆) δ 8.76 (d, J=2.6 Hz, 1H), 8.56 (d, J=2.6 Hz, 1H), 8.10 (d, J=8.4 Hz, 2H), 7.94 (s, 1H), 7.43 (d, J=7.9 Hz, 2H), 2.32 (s, 3H).

### Intermediate 42. (S)-7-(3-(2-(5-Tosyl-5H-pyrrolo[2,3-b]pyrazin-7-yl-6-d)thiazol-4-yl)phenyl)-6,7-dihydro-5H-pyrrolo[1,2-a]imidazol-7-ol

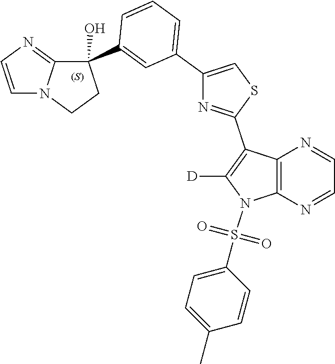

To a microwave tube containing 4-bromo-2-(5-tosyl-5H-pyrrolo[2,3-b]pyrazin-7-yl-6-d)thiazole (Intermediate 41, 200 mg, 0.458 mmol), (S)-7-(3-(4,4,5,5-tetramethyl-1,3,2-dioxaborolan-2-yl)phenyl)-6,7-dihydro-5H-pyrrolo[1,2-a]imidazol-7-ol (Intermediate 39, 230 mg, 0.705 mmol), and K₃PO₄ (300 mg, 1.41 mmol) was added 1,4-dioxane (2.8 mL) and H₂O (0.7 mL). The mixture was purged with Ar for 5 minutes and then Pd(dtbpf)Cl₂ (35 mg, 0.054 mmol) was added. The resultant mixture was purged with Ar for another minutes and then subjected to microwave irradiation at 85° C. for 1 h. The reaction mixture was cooled to room temperature, diluted with water (6 mL), and the aqueous portion was extracted with ethyl acetate:methanol (9/1, 10 mL×5). The combined organic solvent extracts were dried over anhydrous Na₂SO₄, filtered and concentrated to dryness under reduced pressure to yield (S)-7-(3-(2-(5-tosyl-5H-pyrrolo[2,3-b]pyrazin-7-yl-6-d)thiazol-4-yl)phenyl)-6,7-dihydro-5H-pyrrolo[1,2-a]imidazol-7-ol, which was purified by FCC (petroleum ether ethyl acetate=1:0 to 0:1) to yield the title compound (230 mg, 87%) as a yellow solid. LCMS (ESI): mass calcd. for C₂₈H₂₁DN₆O₃S₂, 555.13; m/z found, 556.0 [M+H]⁺.

### Intermediate 43. 5-Tosyl-7-(trimethylstannyl)-5H-pyrrolo[2,3-b]pyrazine-2-d

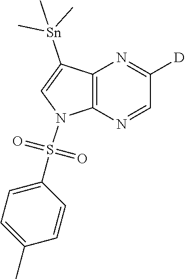 [SMILES: [2H]c1cnc2c(n1)[c]([Sn]([CH3])([CH3])[CH3])cn2S(=O)(=O)c1ccc(C)cc1]

Step A. 5-Tosyl-5H-pyrrolo[2,3-b]pyrazine-2-d. Sodium borodeuteride (2.14 g, 51.1 mmol) was added to a solution of 2-bromo-5-tosyl-5H-pyrrolo[2,3-b]pyrazine (3.0 g, 8.5 mmol), PdCl₂ (151 mg, 0.852 mmol), and deuteromethanol (20 mL) under N₂. The resultant mixture was stirred at room temperature for 16 h. The reaction mixture was concentrated under reduced pressure to yield material which was purified by FCC (eluent: petroleum ether:ethyl acetate=10:1 to 3:1) to yield 5-tosyl-5H-pyrrolo[2,3-b]pyrazine-2-d. (1.2 g, 51%) as a white solid. LCMS (ESI): mass calcd. for C₁₃H₁₀DN₃O₂S, 274.06; m/z found, 275.1 [M+H]⁺. ¹H NMR (400 MHz, DMSO-d₆) δ 8.60 (d, J=2.6 Hz, 0.034H), 8.42 (s, 0.966H), 8.34-8.28 (m, 1H), 8.03-7.97 (m, 2H), 7.43 (d, J=8.2 Hz, 2H), 7.03 (d, J=4.2 Hz, 1H), 2.33 (s, 3H).

Step B. 7-Bromo-5-tosyl-5H-pyrrolo[2,3-b]pyrazine-2-d. NBS (934 mg, 5.25 mmol) was added to a solution of 5-tosyl-5H-pyrrolo[2,3-b]pyrazine-2-d (1.2 g, 4.4 mmol) and DMF (10 mL). The resultant mixture was heated at 50° C. for 16 h, then cooled to room temperature, quenched with water (60 mL) and extracted with ethyl acetate (50 mL×3). The combined organic solvent extracts were dried over anhydrous Na₂SO₄, filtered and concentrated to dryness under reduced pressure to yield material which was purified by FCC (eluent: petroleum ether:ethyl acetate=20:1 to 3:1) to yield 7-bromo-5-tosyl-5H-pyrrolo[2,3-b]pyrazine-2-d (1.1 g, 66%) as a white solid. LCMS (ESI): mass calcd. for C₁₃H₉BrDN₃O₂S, 353.2; m/z found, 355.0 [M+H]⁺.

Step C. 5-Tosyl-7-(trimethylstannyl)-5H-pyrrolo[2,3-b]pyrazine-2-d. To a flask containing 7-bromo-5-tosyl-5H-pyrrolo[2,3-b]pyrazine-2-d (600 mg, 1.70 mmol), 1,1,1,2,2,2-hexamethyldistannane (1.11 g, 3.39 mmol), and toluene (20 mL) was sparged with Ar for 5 minutes and then Pd(PPh₃)₄ (196 mg, 0.17 mmol) was added. The mixture was sparged with Ar for another 5 minutes and then heated at 115° C. for 16 h, then cooled to room temperature, quenched with saturated KF (100 mL) and the aqueous portion was extracted with ethyl acetate (60 mL×3). The combined organic solvent extracts were dried over anhydrous Na₂SO₄, filtered, and concentrated to dryness under reduced pressure to yield 5-tosyl-7-(trimethylstannyl)-5H-pyrrolo[2,3-b]pyrazine-2-d (800 mg), which was used in the next step without further purification. LCMS (ESI): mass calcd. for C₁₆H₁₈DN₃O₂SSn, 438.03; m/z found, 439.0 [M+H]⁺.

### Intermediate 44. 4-Bromo-2-(5-tosyl-5H-pyrrolo[2,3-b]pyrazin-7-yl-2-d)thiazole

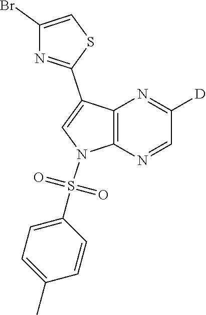 [SMILES: [2H]c1cnc2c(n1)c(-c1nc(Br)cs1)cn2S(=O)(=O)c1ccc(C)cc1]

To a flask containing 5-tosyl-7-(trimethylstannyl)-5H-pyrrolo[2,3-b]pyrazine-2-d (Intermediate 43, 800 mg), 2,4-dibromothiazole (889 mg, 3.66 mmol), and 1,4-dioxane (20 mL) was sparged with Ar for 5 minutes and then Pd(Ph₃P)₄ (211 mg, 0.183 mmol) was added. The mixture was sparged with Ar for another 5 minutes and then heated at 115° C. for 16 h, then cooled to room temperature, quenched with saturated KF (60 mL) and the aqueous portion was extracted with ethyl acetate (80 mL×3). The combined organic solvent extracts were dried over anhydrous Na₂SO₄, filtered, and concentrated to dryness under reduced pressure to yield the product which was purified by FCC (eluent: petroleum ether ethyl acetate=20:1 to 3:1) to yield 4-bromo-2-(5-tosyl-5H-pyrrolo[2,3-b]pyrazin-7-yl-2-d)thiazole (280 mg, 34%) as a brown sold. LCMS (ESI): mass calcd. for C₁₈H₁₀BrDN₄O₂S₂, 434.96; m/z found, 435.9 [M+H]⁺.

### Intermediate 45. (S)-7-(3-(2-(5-Tosyl-5H-pyrrolo[2,3-b]pyrazin-7-yl-2-d)thiazol-4-yl)phenyl)-6,7-dihydro-5H-pyrrolo[1,2-a]imidazol-7-ol

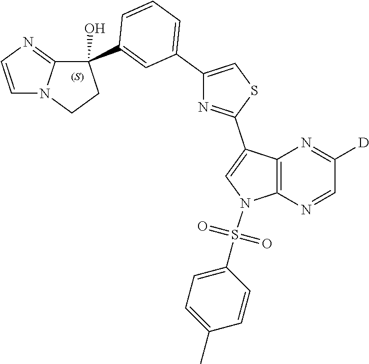

To a 20 mL microwave tube containing 4-bromo-2-(5-tosyl-5H-pyrrolo[2,3-b]pyrazin-7-yl-2-d)thiazole (Intermediate 44, 140 mg, 0.321 mmol), (S)-7-(3-(4,4,5,5-tetramethyl-1,3,2-dioxaborolan-2-yl)phenyl)-6,7-dihydro-5H-pyrrolo[1,2-a]imidazol-7-ol (Intermediate 39, 115 mg, 0.353 mmol), and K₃PO₄ (204 mg, 0.961 mmol) was added 1,4-dioxane (10 mL) and H₂O (2 mL). The mixture was sparged with Ar for 5 minutes and then Pd(dtbpf)Cl₂ (21 mg, 0.03 mmol) was added. The mixture was sparged with Ar for another 5 minutes and the resultant mixture was heated at 80° C. in microwave for 1 h. The reaction mixture was cooled to room temperature, concentrated to dryness, and purified by FCC (petroleum ether:ethyl acetate=10:1 to 0:1, then ethyl acetate:methanol=1:0 to 20:1) to yield (S)-7-(3-(2-(5-tosyl-5H-pyrrolo[2,3-b]pyrazin-7-yl-2-d)thiazol-4-yl)phenyl)-6,7-dihydro-5H-pyrrolo[1,2-a]imidazol-7-ol (130 mg, 68%) as a brown solid. LCMS (ESI): mass calcd. for C₂₈H₂₁DN₆O₃S₂, 555.13; m/z found, 556.1 [M+H]⁺.

### Intermediate 46. (R)-7-(3-(2-(7-tosyl-7H-pyrrolo[2,3-d]pyrimidin-5-yl)thiazol-4-yl)phenyl)-6,7-dihydro-5H-pyrrolo[1,2-a]imidazol-7-ol

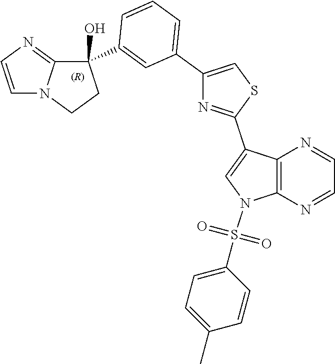

To a microwave tube containing 4-bromo-2-(7-tosyl-7H-pyrrolo[2,3-d]pyrimidin-5-yl)thiazole (Intermediate 7, 140 mg, 0.32 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 100 mg, 0.31 mmol), and K₃PO₄ (214 mg, 1.01 mmol), was added 1,4-dioxane (12 mL) and H₂O (3 mL). The mixture was sparged with Ar for 5 minutes and then Pd(dtbpf)Cl₂ (29 mg, 0.04 mmol) was added. The mixture was sparged with Ar for another 5 minutes and subjected to microwave irradiation at 85° C. for 1 h. The reaction mixture was cooled to room temperature, concentrated to dryness, and purified by FCC (using as a first eluent: petroleum ether:ethyl acetate=1:0 to 0:1, then as a second eluent dichloromethane:methanol=1:0 to 10:1) to yield (R)-7-(3-(2-(7-tosyl-7H-pyrrolo[2,3-d]pyrimidin-5-yl)thiazol-4-yl)phenyl)-6,7-dihydro-5H-pyrrolo[1,2-a]imidazol-7-ol, (80 mg, 30%) as a brown solid. LCMS (ESI): mass calcd. for C₂₈H₂₂N₆O₃S₂, 554.12; m/z found, 555.1 [M+H]⁺.

### Intermediate 47. (S)-7-(3-(2-(7-tosyl-7H-pyrrolo[2,3-d]pyrimidin-5-yl)thiazol-4-yl)phenyl)-6,7-dihydro-5H-pyrrolo[1,2-a]imidazol-7-ol

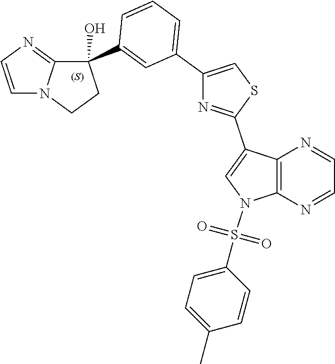

To a microwave tube containing 4-bromo-2-(7-tosyl-7H-pyrrolo[2,3-d]pyrimidin-5-yl)thiazole (Intermediate 7, 130 mg, 0.299 mmol), (S)-7-(3-(4,4,5,5-tetramethyl-1,3,2-dioxaborolan-2-yl)phenyl)-6,7-dihydro-5H-pyrrolo[1,2-a]imidazol-7-ol (Intermediate 39, 97 mg, 0.30 mmol), and K₃PO₄ (190 mg, 0.895 mmol) was added 1,4-dioxane (7.5 mL) and H₂O (1.5 mL). The mixture was sparged with Ar for 5 minutes and then Pd(dtbpf)Cl₂ (19 mg, 0.03 mmol) was added. The mixture was sparged with Ar for another 5 minutes and the resultant mixture was subjected to microwave irradiation at 80° C. for 1 h. The reaction mixture was cooled to room temperature, concentrated to dryness, and purified by FCC (eluent: petroleum ether:ethyl acetate=10:1 to 0:1, then dichloromethane:methanol=1:0 to 20:1) to yield (S)-7-(3-(2-(7-tosyl-7H-pyrrolo[2,3-d]pyrimidin-5-yl)thiazol-4-yl)phenyl)-6,7-dihydro-5H-pyrrolo[1,2-a]imidazol-7-ol (110 mg, 66%) as a brown solid. LCMS (ESI): mass calcd. for C₂₈H₂₂N₆O₃S₂, 554.12; m/z found, 555.1 [M+H]⁺.

### Intermediate 48. (R,S)-7-(3-(4-Bromothiazol-2-yl)phenyl)-6,7-dihydro-5H-cyclopenta[b]pyridin-7-ol

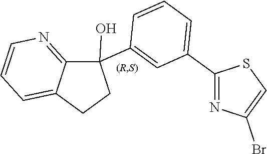

To a microwave tube containing 7-(3-(4,4,5,5-tetramethyl-1,3,2-dioxaborolan-2-yl)phenyl)-6,7-dihydro-5H-cyclopenta[b]pyridin-7-ol, (Intermediate 1, Step B, 800 mg, 1.34 mmol), 2,4-dibromothiazole (488 mg, 2.01 mmol), Cs₂CO₃ (1.3 g, 4.0 mmol) was added 1,4-dioxane (10 mL) and H₂O (2 mL). The mixture was sparged with Ar for 5 minutes and then Pd(dppf)Cl₂ (98 mg, 0.13 mmol) was added. The mixture was sparged with Ar for another 5 minutes and then subjected to microwave irradiation at 85° C. for 1 h. The reaction mixture was cooled to room temperature, and the resulting suspension was combined with another batch of material, filtered through a pad of diatomaceous earth and the pad washed with ethyl acetate (10 mL×2). The filtrate was concentrated to dryness under reduced pressure and purified by FCC (eluent: petroleum ether ethyl acetate=0:1 to 5:1) to yield 7-(3-(4-bromothiazol-2-yl)phenyl)-6,7-dihydro-5H-cyclopenta[b]pyridin-7-ol (600 mg) as a brown solid. LCMS (ESI): mass calcd. for C₁₇H₁₃BrN₂OS, 373.2; m/z found, 375.0 [M+H]⁺.

### Intermediate 49. (R,S)-7-(3-(4-(1-(Phenylsulfonyl)-1H-pyrrolo[2,3-b]pyridin-3-yl)thiazol-2-yl)phenyl)-6,7-dihydro-5H-cyclopenta[b]pyridin-7-ol

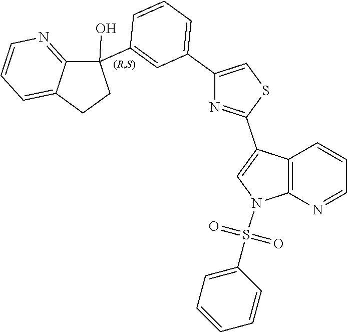

To a microwave tube containing 7-(3-(4-Bromothiazol-2-yl)phenyl)-6,7-dihydro-5H-cyclopenta[b]pyridin-7-ol (500 mg, 1.34 mmol), 1-(phenylsulfonyl)-3-(4,4,5,5-tetramethyl-1,3,2-dioxaborolan-2-yl)-1H-pyrrolo[2,3-b]pyridine (515 mg, 1.34 mmol), and K₃PO₄ (855 mg, 4.03 mmol) was added 1,4-dioxane (10 mL) and H₂O (2.5 mL). The mixture was sparged with Ar for 5 minutes and then Pd(dtbpf)Cl₂ (87 mg, 0.13 mmol) was added. The mixture was sparged with Ar for another 5 minutes and then subjected to microwave irradiation at 80° C. for 1 h. The mixture was cooled to room temperature and combined with another batch of material and filtered through a pad of diatomaceous earth and the pad washed with ethyl acetate (20 mL×2). The filtrate was concentrated to dryness under reduced pressure and purified by FCC (eluent: petroleum ether:ethyl acetate containing 10% methanol=1:0 to 0:1) to yield (R,S)-7-(3-(4-(1-(phenylsulfonyl)-1H-pyrrolo[2,3-b]pyridin-3-yl)thiazol-2-yl)phenyl)-6,7-dihydro-5H-cyclopenta[b]pyridin-7-ol (700 mg) as a brown oil. LCMS (ESI): mass calcd. for C₃₀H₂₂N₄O₃S₂, 550.11; m/z found, 551.1 [M+H]⁺.

### Intermediate 50. 4-Bromo-2-(5-fluoro-1-tosyl-1H-pyrrolo[2,3-b]pyridin-3-yl)thiazole

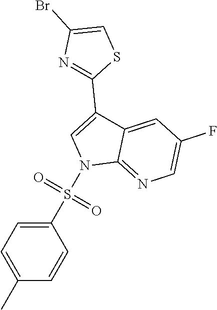 [SMILES: Cc1ccc(S(=O)(=O)n2cc(-c3nc(Br)cs3)c3cc(F)cnc32)cc1]

Step A. 3-Bromo-5-fluoro-1-tosyl-1H-pyrrolo[2,3-b]pyridine. To a flask containing 3-bromo-5 fluoro-1H-pyrrolo[2,3-b]pyridine (1.0 g, 4.7 mmol), DMAP (114 mg, 0.933 mmol), triethylamine (3.2 mL, 23 mmol), and dichloromethane (20 mL) was added TosCl (1.1 g, 5.8 mmol). The resultant mixture was stirred at room temperature for 1 h, then quenched with water (20 mL) and the aqueous portion was extracted with dichloromethane (20 mL×2). The combined organic solvent extracts were dried over anhydrous Na₂SO₄, filtered, concentrated and purified by FCC (eluent: petroleum ether:ethyl acetate=1:0 to 2:1) to yield 3-bromo-5-fluoro-1-tosyl-1H-pyrrolo[2,3-b]pyridine (1.67 g, 88%) as a yellow solid. LCMS (ESI): mass calcd. for C₁₄H₁₀BrFN₂O₂S, 369.21; m/z found, 371.0 [M+H]⁺.

Step B. 5-Fluoro-3-(4,4,5,5-tetramethyl-1,3,2-dioxaborolan-2-yl)-1-tosyl-1H-pyrrolo[2,3-b]pyridine. To a microwave tube containing 3-Bromo-5-fluoro-1-tosyl-1H-pyrrolo[2,3-b]pyridine (500 mg, 1.35 mmol), 4,4,4′,4′,5,5,5′,5′-octamethyl-2,2′-bi(1,3,2-dioxaborolane) (688 mg, 2.71 mmol), and KOAc (398 mg, 4.06 mmol) was added 1,4-dioxane (10 mL). The mixture was sparged with N₂ for 5 minutes and then treated with Pd(dppf)Cl₂ (99 mg, 0.14 mmol). The resultant mixture was heated at 80° C. for 2 h, then cooled to room temperature, and the contents was filtered through a pad of diatomaceous earth and the pad washed with ethyl acetate (10 mL×2). The filtrate was concentrated to dryness and purified by FCC (eluent: petroleum ether:ethyl acetate=1:0 to 10:1) to yield 5-fluoro-3-(4,4,5,5-tetramethyl-1,3,2-dioxaborolan-2-yl)-1-tosyl-1H-pyrrolo[2,3-b]pyridine (800 mg, 91%) as a yellow oil. LCMS (ESI): mass calcd. for C₂₀H₂₂BFN₂O₄S, 416.14; m/z found, 417.1 [M+H]⁺.

Step C. 4-Bromo-2-(5-fluoro-1-tosyl-1H-pyrrolo[2,3-b]pyridin-3-yl)thiazole. To a microwave tube containing 2,4-dibromothiazole (221 mg, 0.910 mmol), 5-fluoro-3-(4,4,5,5-tetramethyl-1,3,2-dioxaborolan-2-yl)-1-tosyl-1H-pyrrolo[2,3-b]pyridine (650 mg, 0.999 mmol), and K₃PO₄ (371 mg, 2.73 mmol) was added 1,4-dioxane (8 mL) and H₂O (2 mL). The mixture was sparged with Ar for 5 minutes and then Pd(dtbpf)Cl₂ (59 mg, 0.091 mmol) was added. The mixture was sparged with Ar for another 5 minutes and then subjected to microwave irradiation at 90° C. for 1 h, then cooled to room temperature, diluted with water (10 mL), and extracted with dichloromethane (10 mL×3). The combined organic solvent extracts were washed with brine (10 mL), dried over anhydrous Na₂SO₄, filtered, concentrated, and purified by FCC (eluent: petroleum ether:ethyl acetate=10:1 to 2:1) to yield 4-bromo-2-(5-fluoro-1-tosyl-1H-pyrrolo[2,3-b]pyridin-3-yl)thiazole (100 mg, 20%) as a white solid. LCMS (ESI): mass calcd. for C₁₇H₁₁BrFN₃O₂S₂, 452.3; m/z found, 454.0 [M+H]⁺. ¹H NMR (400 MHz, DMSO-d₆) δ 8.78 (s, 1H), 8.52 (d, J=1.5 Hz, 1H), 8.29 (dd, J=2.8, 8.5 Hz, 1H), 8.08 (d, J=8.3 Hz, 2H), 7.92 (s, 1H), 7.45 (d, J=8.3 Hz, 2H), 2.36 (s, 3H).

### Intermediate 51. (R)-7-(3-(2-(5-Fluoro-1-tosyl-1H-pyrrolo[2,3-b]pyridin-3-yl)thiazol-4-yl)phenyl)-6,7-dihydro-5H-pyrrolo[1,2-a]imidazol-7-ol

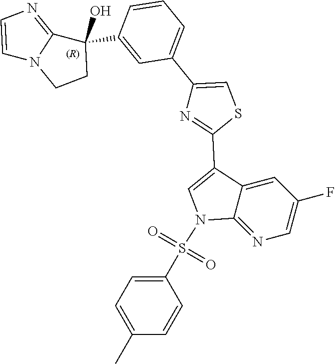

To a microwave tube containing 4-bromo-2-(5-fluoro-1-tosyl-1H-pyrrolo[2,3-b]pyridin-3-yl)thiazole (Intermediate 50, 80 mg, 0.18 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 63 mg, 0.19 mmol), and K₃PO₄ (72 mg, 0.53 mmol) was added 1,4-dioxane (4 mL) and H₂O (1 mL). The mixture was sparged with Ar for 5 minutes and then Pd(dtbpf)Cl₂ (12 mg, 0.018 mmol) was added. The mixture was sparged with Ar for another 5 minutes and then heated at 90° C. for 1 h. The reaction mixture was cooled to room temperature and concentrated to dryness. The product was purified by FCC (eluent: petroleum ether:ethyl acetate=10:1 to 0:1) to yield (R)-7-(3-(2-(5-fluoro-1-tosyl-1H-pyrrolo[2,3-b]pyridin-3-yl)thiazol-4-yl)phenyl)-6,7-dihydro-5H-pyrrolo[1,2-a]imidazol-7-ol (110 mg, 95%) as a yellow solid. LCMS (ESI): mass calcd. for C₂₉H₂₂FN₅O₃S₂, 571.11; m/z found, 572.1 [M+H]⁺.

### Intermediate 52. 4-Bromo-2-(1-tosyl-5-(trifluoromethyl)-1H-pyrrolo[2,3-b]pyridin-3-yl)thiazole

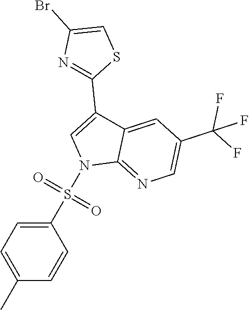 [SMILES: Cc1ccc(S(=O)(=O)n2cc(-c3nc(Br)cs3)c3cc(C(F)(F)F)cnc32)cc1]

Step A. 3-Bromo-1-tosyl-5-(trifluoromethyl)-1H-pyrrolo[2,3-b]pyridine. To a flask containing 3-bromo-5-(trifluoromethyl)-1H-pyrrolo[2,3-b]pyridine (1.0 g, 3.8 mmol), triethylamine (0.79 ml, 5.7 mmol), and dichloromethane (10 mL) was added TosCl (865 mg, 4.54 mmol). The resultant mixture was stirred at room temperature for 12 h. The mixture was concentrated to dryness under reduced pressure to yield the product, which was purified by FCC (eluent: petroleum ether:ethyl acetate=1:0 to 6:1) to yield 3-bromo-1-tosyl-5-(trifluoromethyl)-1H-pyrrolo[2,3-b]pyridine (1.6 g, 79%) as a white solid. LCMS (ESI): mass calcd. for C₁₅H₁₀BrF₃N₂O₂S, 417.96; m/z found, 420.9 [M+H]⁺.

Step B. 3-(4,4,5,5-Tetramethyl-1,3,2-dioxaborolan-2-yl)-1-tosyl-5-(trifluoromethyl)-1H-pyrrolo[2,3-b]pyridine. To a microwave tube containing 3-bromo-1-tosyl-5-(trifluoromethyl)-1H-pyrrolo[2,3-b]pyridine (900 mg, 1.68 mmol), 4,4,4′,4′,5,5,5′,5′-octamethyl-2,2′-bi(1,3,2-dioxaborolane) (850 mg, 3.35 mmol), and KOAc (493 mg, 5.02 mmol) was added 1,4-dioxane (10 mL). The mixture was sparged with Ar for 5 minutes and then Pd(dppf)Cl₂ (123 mg, 0.168 mmol) was added. The mixture was sparged with Ar for another 5 minutes and then subjected to microwave irradiation at 90° C. for 1 h. The reaction mixture was cooled to room temperature and the suspension was filtered through a pad of diatomaceous earth and the pad washed with ethyl acetate (50 mL). The filtrate concentrated to dryness under reduced pressure to yield the product, which was purified by FCC (eluent: petroleum ether ethyl acetate=0:1 to 5:1) to yield 3-(4,4,5,5-tetramethyl-1,3,2-dioxaborolan-2-yl)-1-tosyl-5-(trifluoromethyl)-1H-pyrrolo[2,3-b]pyridine (1.0 g) as a yellow oil. LCMS (ESI): mass calcd. for C₂₁H₂₂BF₃N₂O₄S₂, 466.13; m/z found, 467.2 [M+H]⁺.

Step C. 4-Bromo-2-(1-tosyl-5-(trifluoromethyl)-1H-pyrrolo[2,3-b]pyridin-3-yl)thiazole. To a microwave tube containing 3-(4,4,5,5-Tetramethyl-1,3,2-dioxaborolan-2-yl)-1-tosyl-5-(trifluoromethyl)-1H-pyrrolo[2,3-b]pyridine (1.0 g, 0.664 mmol), 2,4-dibromothiazole (242 mg, 0.996 mmol), and CS₂CO₃ (649 mg, 1.99 mmol) was added 1,4-dioxane (10 mL), and H₂O (2 mL). The mixture was sparged with Ar for 5 minutes and then Pd(dppf)Cl₂ (49 mg, 0.067 mmol) was added. The mixture was sparged with Ar for another 5 minutes and subjected to microwave irradiation at 85° C. for 1 h. The reaction mixture was cooled to room temperature and the suspension was filtered through a pad of diatomaceous earth and the pad washed with ethyl acetate (50 mL). The filtrate was concentrated to dryness under reduced pressure to yield the product, which was purified by FCC (eluent: petroleum ether ethyl acetate=0:1 to 5:1) to yield 4-bromo-2-(1-tosyl-5-(trifluoromethyl)-1H-pyrrolo[2,3-b]pyridin-3-yl)thiazole (1.0 g) as a brown solid. LCMS (ESI): mass calcd. for C₁₈H₁₁BrF₃N₃O₂S₂, 502.32; m/z found, 504.0 [M+H]⁺.

### Intermediate 53. (R)-7-(3-(2-(1-Tosyl-5-(trifluoromethyl)-1H-pyrrolo[2,3-b]pyridin-3-yl)thiazol-4-yl)phenyl)-6,7-dihydro-5H-pyrrolo[1,2-a]imidazol-7-ol

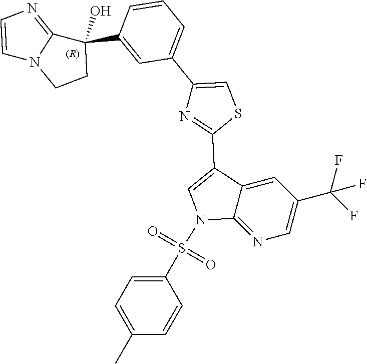

To a microwave tube containing 4-bromo-2-(1-tosyl-5-(trifluoromethyl)-1H-pyrrolo[2,3-b]pyridin-3-yl)thiazole (448 mg, 0.321 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 100 mg, 0.31 mmol), and K₃PO₄ (205 mg, 0.966 mmol) was added, 1,4-dioxane (10 mL) and H₂O (2.5 mL). The mixture was sparged with Ar for 5 minutes and then Pd(dtbpf)Cl₂ (21 mg, 0.032 mmol) was added. The mixture was sparged with Ar for another 5 minutes and then subjected to microwave irradiation at 80° C. for 1 h. The reaction mixture was cooled to room temperature, filtered through a pad of diatomaceous earth and the pad washed with ethyl acetate (50 mL). The filtrate was concentrated to dryness under reduced pressure. The product was purified by FCC (eluent: petroleum ether:ethyl acetate (ethyl acetate contained 10% methanol)=1:0 to 0:1) to yield (R)-7-(3-(2-(1-tosyl-5-(trifluoromethyl)-1H-pyrrolo[2,3-b]pyridin-3-yl)thiazol-4-yl)phenyl)-6,7-dihydro-5H-pyrrolo[1,2-a]imidazol-7-ol (140 mg, 61%) as a brown solid. LCMS (ESI): mass calcd. for C₃₀H₂₂F₃N₅O₃S₂, 621.11; m/z found, 622.1 [M+H]⁺.

### Intermediate 54. 3-(4-Bromothiazol-2-yl)-1-tosyl-1H-pyrrolo[2,3-b]pyridine-5-carbonitrile

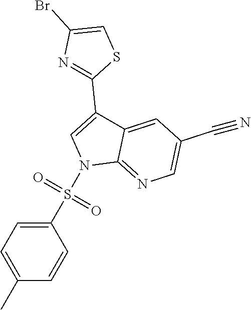 [SMILES: Cc1ccc(S(=O)(=O)n2cc(-c3nc(Br)cs3)c3cc(C#N)cnc32)cc1]

Step A. 3-Bromo-1-tosyl-1H-pyrrolo[2,3-b]pyridine-5-carbonitrile. To a flask containing 3-bromo-1H-pyrrolo[2,3-b]pyridine-5-carbonitrile (500 mg, 2.25 mmol), DMAP (55 mg, 0.45 mmol), triethylamine (0.94 ml, 6.8 mmol), and dichloromethane (15 mL) was added TosCl (515 mg, 2.70 mmol). The resultant mixture was stirred at room temperature for 15 h, then diluted with H₂O (50 mL) and extracted with dichloromethane (50 mL×3). The combined organic solvent extracts were dried over anhydrous Na₂SO₄, filtered, and concentrated to dryness under reduced pressure to yield the product, which was purified by FCC (eluent: petroleum ether:ethyl acetate=1:0 to 5:1) to yield 3-bromo-1-tosyl-1H-pyrrolo[2,3-b]pyridine-5-carbonitrile (680 mg, 73%) as a white solid. LCMS (ESI): mass calcd. for C₁₅H₁₀BrN₃O₂S, 376.22; m/z found, 378.0 [M+H]⁺.

Step B. 1-(3-(2-(5H-Pyrrolo[2,3-b]pyrazin-7-yl)thiazol-4-yl)phenyl)-1-(1H imidazol-2-yl)ethanol. To a flask containing 4,4,4′,4′,5,5,5′,5′-octamethyl-2,2′-bi(1,3,2-dioxaborolane) (810 mg, 3.19 mmol), and KOAc (470 mg, 4.79 mmol), and 3-bromo-1-tosyl-1H-pyrrolo[2,3-b]pyridine-5-carbonitrile (600 mg, 1.60 mmol) was added 1,4-dioxane (20 mL). The mixture was sparged with N₂ for 5 minutes and then Pd(dppf)Cl₂ (117 mg, 0.160 mmol) was added. The resultant mixture was heated at 85° C. for 16 h. The reaction mixture was cooled to room temperature, combined with another batch of material, and the suspension was filtered through a pad of diatomaceous earth, poured into water (80 mL), and extracted with dichloromethane (150 mL×3). The combined organic solvent extracts were washed with brine (10 mL), dried over anhydrous Na₂SO₄, filtered, and concentrated to dryness under reduced pressure to yield the product which was purified by FCC (eluent: petroleum ether:ethyl acetate=20:1 to 3:1) to yield 1-(3-(2-(5H-pyrrolo[2,3-b]pyrazin-7-yl)thiazol-4-yl)phenyl)-1-(1H-imidazol-2-yl)ethanol (600 mg) as a white solid. LCMS (ESI): mass calcd. for C₂₁H₂₂BN₃O₄S, 423.14; m/z found, 423.9 [M+H]⁺.

Step C. 3-(4-Bromothiazol-2-yl)-1-tosyl-1H-pyrrolo[2,3-b]pyridine-5-carbonitrile. To a microwave tube containing 3-(4,4,5,5-Tetramethyl-1,3,2-dioxaborolan-2-yl)-1-tosyl-1H-pyrrolo[2,3-b]pyridine-5-carbonitrile (600 mg, 1.42 mmol), 2,4-dibromothiazole (516 mg, 2.12 mmol), and Cs₂CO₃ (1.16 g, 3.56 mmol) was added 1,4-dioxane (10 mL), and H₂O (1 mL). The mixture was sparged with Ar for 5 minutes and then Pd(dppf)₂Cl₂ (104 mg, 0.142 mmol) was added. The mixture was sparged with Ar for another 5 minutes and then subjected to microwave irradiation at 85° C. for 1 h. The reaction mixture was cooled to room temperature, and the suspension was filtered through a pad of diatomaceous earth and the pad washed with ethyl acetate (50 mL). The filtrate was concentrated to dryness under reduced pressure to yield the product, which was purified by FCC (eluent: petroleum ether ethyl acetate=0:1 to 3:1) to yield 3-(4 bromothiazol-2-yl)-1-tosyl-1H-pyrrolo[2,3-b]pyridine-5-carbonitrile (400 mg, 57%) as a brown solid. LCMS (ESI): mass calcd. for C₁₈H₁₁BrN₄O₂S₂, 459.34; m/z found, 461.0 [M+H]⁺. ¹H NMR (400 MHz, CDCl₃) δ 8.96 (d, J=3.3 Hz, 1H), 8.74 (d, J=3.1 Hz, 1H), 8.38 (d, J=5.3 Hz, 1H), 8.14 (dd, J=4.5, 7.6 Hz, 2H), 7.35 (d, J=7.5 Hz, 2H), 7.26 (s, 1H), 2.43 (d, J=4.6 Hz, 3H).

### Intermediate 55. (R)-3-(4-(3-(7-hydroxy-6,7-dihydro-5H-pyrrolo[1,2-a]imidazol-7-yl)phenyl)thiazol-2-yl)-1-tosyl-1H-pyrrolo[2,3-b]pyridine-5-carbonitrile

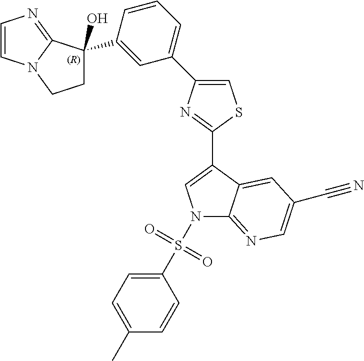

To a tube containing 3-(4-bromothiazol-2-yl)-1-tosyl-1H-pyrrolo[2,3-b]pyridine-5-carbonitrile (Intermediate 54, 130 mg, 0.28 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 92 mg, 0.28 mmol), and K₃PO₄ (116 mg, 0.852 mmol) was added, 1,4-dioxane (4 mL) and H₂O (1 mL). The mixture was sparged with Ar for 5 minutes and then Pd(dtbpf)Cl₂ (18 mg, 0.028 mmol) was added. The mixture was sparged with Ar for another 5 minutes and the resultant mixture was subjected to microwave irradiation at 85° C. for 1 h. The reaction mixture was cooled to room temperature and concentrated to dryness under reduced pressure. The product was purified by FCC (eluent: petroleum ether:ethyl acetate=10:1 to 0:1) to yield (R)-3-(4-(3-(7-hydroxy-6,7-dihydro-5H-pyrrolo[1,2-a]imidazol-7-yl)phenyl)thiazol-2-yl)-1-tosyl-1H-pyrrolo[2,3-b]pyridine-5-carbonitrile (110 mg, 56%) as a brown solid. LCMS (ESI): mass calcd. for C₃₀H₂₂N₆O₃S₂, 578.12; m/z found, 579.1 [M+H]⁺.

### Intermediate 56. 4-Bromo-2-(5-chloro-1-tosyl-1H-pyrrolo[2,3-b]pyridin-3-yl)thiazole

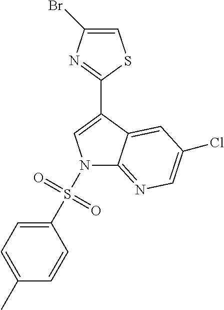 [SMILES: Cc1ccc(S(=O)(=O)n2cc(-c3nc(Br)cs3)c3cc(Cl)cnc32)cc1]

Step A. 3-Bromo-5-chloro-1-tosyl-1H-pyrrolo[2,3-b]pyridine. To a flask containing 3-bromo-5-chloro-1H-pyrrolo[2,3-b]pyridine (2.0 g, 8.6 mmol), triethylamine (1.4 mL, 10 mmol) in dichloromethane (40 mL) was added TosCl (1.9 g, 10 mmol) and DMAP (106 mg, 0.868 mmol). The reaction mixture was stirred at room temperature for 12 h. The mixture was then washed with ethyl acetate (50 mL), filtered, and concentrated to dryness under reduced pressure to yield the product, which was purified by FCC (eluent: petroleum ether:ethyl acetate=10:1) to yield 4-bromo-2-(5-chloro-1-tosyl-1H-pyrrolo[2,3-b]pyridin-3-yl)thiazole (2.3 g, 48.45%) as a brown solid. LCMS (ESI): mass calcd. for C₁₄H₁₀BrClN₂O₂S, 385.66; m/z found, 387.0 [M+H]⁺.

Step B. 5-Chloro-3-(4,4,5,5-tetramethyl-1,3,2-dioxaborolan-2-yl)-1-tosyl-1H-pyrrolo[2,3-b]pyridine. To a flask containing 3-bromo-5-chloro-1-tosyl-1H-pyrrolo[2,3-b]pyridine (1.00 g, 2.59 mmol), 4,4,4′,4′,5,5,5′,5′-octamethyl-2,2′-bi(1,3,2-dioxaborolane) (988 mg, 3.89 mmol), K₂CO₃ (1.08 g, 7.81 mmol), was added 1,4-dioxane (10 mL). The mixture was sparged with Ar for 5 minutes and then Pd(dppf)Cl₂ (285 mg, 0.390 mmol) was added. The mixture was sparged with Ar for another 5 minutes and the resultant mixture was heated at 90° C. for 16 h. The contents was cooled to room temperature and the suspension was filtered through a pad of diatomaceous earth and the pad washed with ethyl acetate (60 mL). The filtrate was concentrated to dryness under reduced pressure to yield the product, which was purified by FCC (elunet: petroleum ether:ethyl acetate=4:1 to 1:2) to yield 5-chloro-3-(4,4,5,5-tetramethyl-1,3,2-dioxaborolan-2-yl)-1-tosyl-1H-pyrrolo[2,3-b]pyridine (737 mg, 42.0% purity, 65.7%) as a brown solid. LCMS (ESI): mass calcd. for C₂₀H₂₂BClN₂O₄S, 432.11; m/z found, 433.1 [M+H]⁺.

Step C. 4-Bromo-2-(5-chloro-1-tosyl-1H-pyrrolo[2,3-b]pyridin-3-yl)thiazole. To a microwave tube containing 5-Chloro-3-(4,4,5,5-tetramethyl-1,3,2-dioxaborolan-2-yl)-1-tosyl-1H-pyrrolo[2,3-b]pyridine (600 mg, 1.39 mmol), 2,4-dibromothiazole (404 mg, 1.66 mmol), Cs₂CO₃ (1.36 g, 4.16 mmol), was added 1,4-dioxane (10 mL), and H₂O (2 mL). The mixture was sparged with Ar for 5 minutes and then Pd(dppf)Cl₂ (101 mg, 0.14 mmol) was added. The mixture was sparged with Ar for another 5 minutes and subjected to microwave irradiation at 85° C. for 1 h. The contents was cooled to room temperature, combined with another batch of material, filtered, and concentrated to dryness under reduced pressure to yield the product, which was purified by FCC (eluent: petroleum ether:ethyl acetate=10:1 to 1:2) to yield 4-bromo-2-(5-chloro-1-tosyl-1H-pyrrolo[2,3-b]pyridin-3-yl)thiazole (170 mg, 11%) as a white solid. LCMS (ESI): mass calcd. for C₁₇H₁₁BrClN₃O₂S₂, 468.77; m/z found, 469.9 [M+H]⁺.

### Intermediate 57. (R)-7-(3-(2-(5-Chloro-1-tosyl-1H-pyrrolo[2,3-b]pyridin-3-yl)thiazol-4-yl)phenyl)-6,7-dihydro-5H-pyrrolo[1,2-a]imidazol-7-ol

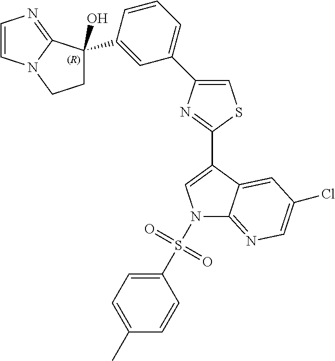

To a microwave tube containing 4-bromo-2-(7-tosyl-7H-pyrrolo[2,3-d]pyrimidin-5-yl)thiazole (Intermediate 7, 170 mg, 0.36 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 118 mg, 0.362 mmol), and K₃PO₄ (231 mg, 1.09 mmol) was added 1,4-dioxane (3 mL) and H₂O (0.6 mL). The mixture was sparged with Ar for 5 minutes and then Pd(dtbpf)Cl₂ (24 mg, 0.037 mmol) was added. The mixture was sparged with Ar for another 5 minutes and subjected to microwave irradiation at 80° C. for 1 h. The reaction mixture was cooled to room temperature and concentrated to dryness under reduced pressure. The product was purified by FCC (using as the initial eluent: petroleum ether ethyl acetate=10:1 to 0:1, then a second eluent: ethyl acetate:methanol=1:0 to 10:1) to yield (R)-7-(3-(2-(5-chloro-1-tosyl-1H-pyrrolo[2,3-b]pyridin-3-yl)thiazol-4-yl)phenyl)-6,7-dihydro-5H-pyrrolo[1,2-a]imidazol-7-ol (100 mg, 38%) as a brown solid. LCMS (ESI): mass calcd. for C₂₉H₂₂ClN₅O₃S₂, 587.09; m/z found, 588.0 [M+H]⁺.

### Intermediate 58. 4-bromo-2-(5-(methylsulfonyl)-1-tosyl-1H-pyrrolo[2,3-b]pyridin-3-yl)thiazole

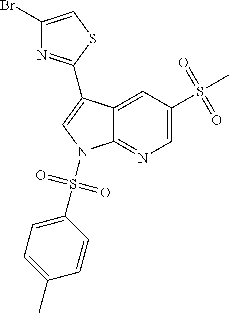 [SMILES: Cc1ccc(S(=O)(=O)n2cc(-c3nc(Br)cs3)c3cc(S(C)(=O)=O)cnc32)cc1]

Step A. 5-Bromo-1-tosyl-1H-pyrrolo[2,3-b]pyridine. To a flask containing 5-bromo-1H-pyrrolo[2,3-b]pyridine (15.0 g, 76.1 mmol), triethylamine (42.0 mL, 302 mmol), DMAP (1.9 g, 16 mmol), and dichloromethane (300 mL) was added TosCl (19.0 g, 99.7 mmol). The resultant mixture was stirred at room temperature for 2 h, then quenched with H₂O (200 mL) and extracted with dichloromethane (300 mL×3). The combined organic solvent extracts were washed with brine (50 mL×3), dried over anhydrous Na₂SO₄, filtered, and concentrated to dryness under reduced pressure to yield the product, which was purified by FCC (eluent: petroleum ether:ethyl acetate=1:0 to 0:1) to yield 5-bromo-1-tosyl-1H-pyrrolo[2,3-b]pyridine (25 g, 90%) as a yellow solid. LCMS (ESI): mass calcd. for C₁₄H₁₁BrN₂O₂S, 351.21; m/z found, 352.9 [M+H]⁺.

Step B. 5-(Methylsulfonyl)-1-tosyl-1H-pyrrolo[2,3-b]pyridine. L-proline (805 mg, 6.99 mmol), CuI (1.36 g, 7.14 mmol), NaSO₂Me (5.3 g, 52 mmol), and NaOH (274 mg, 6.85 mmol) were added to a solution of 5-bromo-1H-pyrrolo[2,3-b]pyridine (12 g, 34 mmol) and DMSO (150 mL) under a N₂ atmosphere. This reaction mixture was heated at 120° C. for 16 h. The reaction mixture was then cooled to room temperature, concentrated to dryness under reduced pressure, diluted with H₂O (300 mL), and extracted with ethyl acetate (400 mL×3). The combined organic solvent extracts were washed with brine (50 mL×3), dried over anhydrous Na₂SO₄, filtered, and concentrated to yield the product, which was purified by FCC (eluent: petroleum ether:ethyl acetate=1:0 to 0:1) to yield 5-(methylsulfonyl)-1-tosyl-1H-pyrrolo[2,3-b]pyridine (1.4 g, 11%) as a yellow solid. LCMS (ESI), mass calcd. for C₁₅H₁₄N₂O₄S₂, 350.04; m/z found, 351.4 [M+H]⁺. ¹H NMR (400 MHz, DMSO-d₆) δ 8.86 (d, J=2.3 Hz, 1H), 8.62 (d, J=2.0 Hz, 1H), 8.15 (d, J=4.0 Hz, 1H), 8.05 (d, J=8.5 Hz, 2H), 7.46 (d, J=8.3 Hz, 2H), 7.02 (d, J=4.0 Hz, 1H), 3.31 (s, 3H), 2.36 (s, 3H).

Step C. 3-Bromo-5-(methylsulfonyl)-1-tosyl-1H-pyrrolo[2,3-b]pyridine. A flask containing 5-(methanesulfonyl)-1-tosyl-1H-pyrrolo[2,3-b]pyridine (1.4 g, 3.36 mmol) and DMF (50 mL) was cooled to 0° C. and then NBS (717 mg, 4.03 mmol) was added. The reaction mixture was heated at 50° C. for 16 h. The reaction mixture was then cooled to room temperature and the product was isolated via filtration. The filter cake was washed ethyl acetate (10 mL) and dried under reduced pressure to yield 3-bromo-5-(methylsulfonyl)-1-tosyl-1H-pyrrolo[2,3-b]pyridine (800 mg, 54%) as a white solid. LCMS (ESI): mass calcd. for C₁₅H₁₃BrN₂O₄S₂, 429.39; m/z found, 431.0 [M+H]⁺. ¹H NMR (400 MHz, DMSO-d₆) δ 8.93 (d, J=2.0 Hz, 1H), 8.49 (s, 1H), 8.42 (d, J=2.0 Hz, 1H), 8.07 (d, J=8.3 Hz, 2H), 7.47 (d, J=8.3 Hz, 2H), 3.36 (s, 3H), 2.37 (s, 3H).

Step D. 5-(Methylsulfonyl)-3-(4,4,5,5-tetramethyl-1,3,2-dioxaborolan-2-yl)-1-tosyl-1H-pyrrolo[2,3-b]pyridine. 3-Bromo-5-(methanesulfonyl)-1-tosyl-1H-pyrrolo[2,3-b]pyridine (800 mg, 1.86 mmol) was added to a mixture of 4,4,4′,4′,5,5,5′,5′-octamethyl-2,2′-bi(1,3,2-dioxaborolane) (1.20 g, 4.73 mmol), KOAc (549 mg, 5.59 mmol), and THE (100 mL). The mixture was sparged with Ar for 5 minutes and then treated with Pd(dppf)Cl₂ (136 mg, 0.186 mmol). The mixture was sparged with Ar for another 5 minutes and then heated at 70° C. under Ar atmosphere for 20 h. The reaction mixture was cooled to room temperature, concentrated, and purified by FCC (eluent: petroleum ether:ethyl acetate=1:0 to 0:1) to yield 5-(methylsulfonyl)-3-(4,4,5,5-tetramethyl-1,3,2-dioxaborolan-2-yl)-1-tosyl-1H-pyrrolo[2,3-b]pyridine (300 mg, 25%) as a brown solid. LCMS (ESI): mass calcd. for C₂₁H₂₅BN₂O₆S₂, 476.12; m/z found, 477.2 [M+H]⁺.

Step E. 4-Bromo-2-(5-(methylsulfonyl)-1-tosyl-1H-pyrrolo[2,3-b]pyridin-3-yl)thiazole. To a tube containing 5-(Methylsulfonyl)-3-(4,4,5,5-tetramethyl-1,3,2-dioxaborolan-2-yl)-1-tosyl-1H-pyrrolo[2,3-b]pyridine (300 mg, 0.630 mmol), 2,4-dibromothiazole (199 mg, 0.819 mmol), Cs₂CO₃ (479 mg, 1.47 mmol) was added 1,4-dioxane (12.5 mL) and H₂O (2.5 mL). The mixture was sparged with Ar for 5 minutes and then Pd(dppf)Cl₂ (48 mg, 0.066 mmol) was added. The mixture was sparged with Ar for another 5 minutes and then subjected to microwave irradiation at 80° C. for 1 h. The reaction mixture was cooled to room temperature, concentrated, and initially purified by FCC (eluent: petroleum ether:ethyl acetate=1:0 to 0:1). The product was further purified by preparative HPLC using an HPLC column, such as a Phenomenex Gemini NX-C18 75 mm×30 mm×3 μm column (eluent: 53% to 83%, CH₃CN and H₂O (with 10 mM NH₄HCO₃)) to yield the product. The product was then suspended in water (10 mL) and the mixture was frozen by insertion into a −78° C. bath. The frozen mixture was then lyophilized to yield 4-bromo-2-(5-(methylsulfonyl)-1-tosyl-1H-pyrrolo[2,3-b]pyridin-3-yl)thiazole (80 mg, 25%) as a white solid. LCMS (ESI): mass calcd. for C₁₈H₁₄BrN₃O₄S₃, 510.93; m/z found, 511.9 [M+H]⁺.

### Intermediate 59. (R)-7-(3-(2-(5-(Methylsulfonyl)-1-tosyl-1H-pyrrolo[2,3-b]pyridin-3-yl)thiazol-4-yl)phenyl)-6,7-dihydro-5H-pyrrolo[1,2-a]imidazol-7-ol

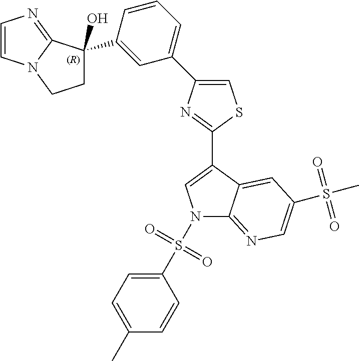

To a tube containing 4-bromo-2-(5-(methylsulfonyl)-1-tosyl-1H-pyrrolo[2,3-b]pyridin-3-yl)thiazole (Intermediate 58, 130 mg, 0.254 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 100 mg, 0.31 mmol), and K₃PO₄ (162 mg, 0.763 mmol) was added 1,4-dioxane (12 mL) and H₂O (3 mL). The mixture was sparged with Ar for 5 minutes and then Pd(dtbpf)Cl₂ (17 mg, 0.026 mmol) was added. The mixture was sparged with Ar for another 5 minutes and then subjected to microwave irradiation at 70° C. for 1 h. The contents were cooled to room temperature and concentrated. The product was purified by FCC (eluent: petroleum ether:ethyl acetate=1:0 to 0:1) to yield (R)-7-(3-(2-(5-(methylsulfonyl)-1-tosyl-1H-pyrrolo[2,3-b]pyridin-3-yl)thiazol-4-yl)phenyl)-6,7-dihydro-5H-pyrrolo[1,2-a]imidazol-7-ol (100 mg, 62%) as a brown oil. LCMS (ESI): mass calcd. for C₃₀H₂₅N₅O₅S₃, 631.10; m/z found, 632.3 [M+H]⁺.

### Intermediate 60. 4-Bromo-2-(4-fluoro-1-tosyl-1H-pyrrolo[2,3-b]pyridin-3-yl)thiazole

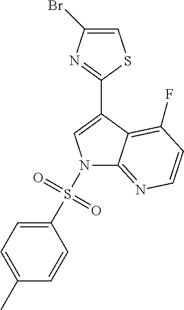 [SMILES: Cc1ccc(S(=O)(=O)n2cc(-c3nc(Br)cs3)c3c(F)ccnc32)cc1]

Step A. 4-Fluoro-1-tosyl-1H-pyrrolo[2,3-b]pyridine. To a flask 4-fluoro-1H-pyrrolo[2,3-b]pyridine (2.0 g, 15 mmol), DMAP (359 mg, 2.94 mmol), triethylamine (10 ml, 72 mmol), and dichloromethane (50 mL) was added containing TosCl (3.36 g, 17.6 mmol). The resultant mixture was stirred at room temperature for 11 h. The mixture was concentrated and purified by FCC (eluent: petroleum ether:ethyl acetate=20:1 to 3:1) to yield 4-fluoro-1-tosyl-1H-pyrrolo[2,3-b]pyridine (3.5 g, 72%) as a white solid. LCMS (ESI): mass calcd. for C₁₄H₁₁FN₂O₂S, 290.05; m/z found, 291.1 [M+H]⁺. ¹H NMR (400 MHz, CDCl₃) δ 8.96 (d, J=3.3 Hz, 1H), 8.74 (d, J=3.1 Hz, 1H), 8.38 (d, J=5.3 Hz, 1H), 8.14 (dd, J=7.6, 4.5 Hz, 2H), 7.35 (d, J=7.5 Hz, 2H), 7.26 (s, 1H), 2.43 (d, J=4.6 Hz, 3H).

Step B. 3-Bromo-4-fluoro-1-tosyl-1H-pyrrolo[2,3-b]pyridine. To a flask containing 4 fluoro-1-tosyl-1H-pyrrolo[2,3-b]pyridin (3.5 g, 12 mmol) and DMF (40 mL) was added NBS (2.36 g, 13.3 mmol). The resultant mixture was heated at 80° C. for 16 h, cooled to room temperature and concentrated to yield the product, which was purified by FCC (eluent: petroleum ether:ethyl acetate=20:1 to 3:1) to yield 3-bromo-4-fluoro-1-tosyl-1H-pyrrolo[2,3-b]pyridine (2.5 g, 42%) as a pink solid. LCMS (ESI): mass calcd. for C₁₄H₁₀BrFN₂O₂S, 367.96; m/z found, 369.0 [M+H]⁺.

Step C. 4-Fluoro-1-tosyl-3-(trimethylstannyl)-1H-pyrrolo[2,3-b]pyridine. A flask containing 3-Bromo-4-fluoro-1-tosyl-1H-pyrrolo[2,3-b]pyridine (2.0 g, 5.4 mmol), 1,1,1,2,2,2-hexamethyldistannane (3.690 g, 11.26 mmol), and toluene (30 mL) was sparged with Ar for 5 minutes and then treated with Pd(PPh₃)₄ (1.25 g, 1.08 mmol). The mixture was sparged with Ar for another 5 minutes and then heated at 115° C. for 3 h. The reaction mixture was cooled to room temperature, quenched with saturated. KF (200 mL) and extracted with ethyl acetate (300 mL×3). The combined organic solvent extracts were dried over anhydrous Na₂SO₄, filtered, and concentrated to yield the product (3.3 g) as a yellow oil, which was used in the next step without further purification.

Step D. 4-Bromo-2-(4-fluoro-1-tosyl-1H-pyrrolo[2,3-b]pyridin-3-yl)thiazole. To a flask containing 4-Fluoro-1-tosyl-3-(trimethylstannyl)-1H-pyrrolo[2,3-b]pyridine (1.5 g, 3.3 mmol), 2,4-dibromothiazole (1.6 g, 6.6 mmol), was added 1,4-dioxane (30 mL). The mixture was sparged with Ar for 5 minutes and then treated with Pd(Ph₃P)₄ (383 mg, 0.331 mmol). The mixture was sparged with Ar for another 5 minutes and then heated at 120° C. for 16 h. The reaction mixture was cooled to room temperature, filtered through a pad of diatomaceous earth, and the pad washed with ethyl acetate (50 mL). The filtrate was concentrated and purified by FCC (eluent: petroleum ether:ethyl acetate=1:0 to 5:1) to yield 4-bromo-2-(4 fluoro-1-tosyl-1H-pyrrolo[2,3-b]pyridin-3-yl)thiazole (200 mg) as a brown solid. LCMS (ESI): mass calcd. for C₁₇H₁₁BrFN₃O₂S₂, 452.32; m/z found, 454.1 [M+H]⁺. ¹H NMR (400 MHz, DMSO-d₆) δ 8.57-8.46 (m, 2H), 8.12 (d, J=8.4 Hz, 2H), 7.94 (s, 1H), 7.46 (d, J=7.9 Hz, 2H), 7.37 (dd, J=5.6, 10.7 Hz, 1H), 2.36 (5, 3H).

### Intermediate 61. (R)-7-(3-(2-(4-Fluoro-1-tosyl-1H-pyrrolo[2,3-b]pyridin-3-yl)thiazol-4-yl)phenyl)-6,7-dihydro-5H-pyrrolo[1,2-a]imidazol-7-ol

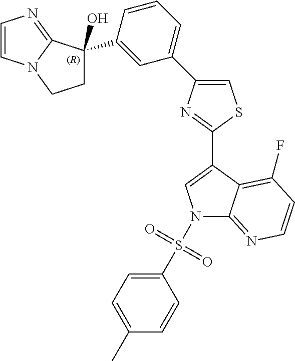

To a microwave tube containing 4-bromo-2-(4-fluoro-1-tosyl-1H-pyrrolo[2,3-b]pyridin-3-yl)thiazole (Intermediate 60, 130 mg, 0.29 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 94 mg, 0.29 mmol), and K₃PO₄ (117 mg, 0.860 mmol) was added 1,4-dioxane (4 mL), and H₂O (1 mL). The mixture was sparged with Ar for 5 minutes and then treated with Pd(dtbpf)Cl₂ (19 mg, 0.029 mmol). The mixture was sparged with Ar for another 5 minutes and subjected to microwave irradiation at 90° C. for 1 h. The reaction mixture was cooled to room temperature and concentrated. The product was purified by FCC (eluent: petroleum ether:ethyl acetate=10:1 to 0:1) to yield (R)-7-(3-(2-(4-fluoro-1-tosyl-1H-pyrrolo[2,3-b]pyridin-3-yl)thiazol-4-yl)phenyl)-6,7-dihydro-5H-pyrrolo[1,2-a]imidazol-7-ol (110 mg, 64%) as a brown solid. LCMS (ESI): mass calcd. for C₂₉H₂₂FN₅O₃S₂, 571.11; m/z found, 572.1 [M+H]⁺.

### Intermediate 62. (R)-7-(3-(5-(1-Tosyl-1H-pyrrolo[2,3-b]pyridin-3-yl)furan-3-yl)phenyl)-6,7-dihydro-5H-pyrrolo[1,2-a]imidazol-7-ol

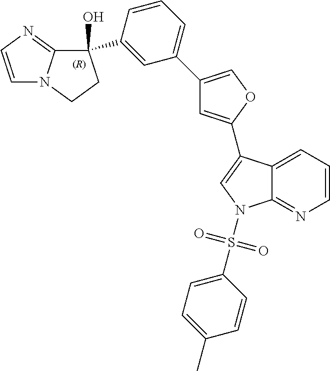

Step A. 3-(4-Bromofuran-2-yl)-1-tosyl-1H-pyrrolo[2,3-b]pyridine. To a microwave tube containing 3-bromo-1-tosyl-1H-pyrrolo[2,3-b]pyridine (500 mg, 1.4 mmol), 2-(4-bromofuran-2-yl)-4,4,5,5-tetramethyl-1,3,2-dioxaborolane (311 mg, 1.14 mmol), and K₃PO₄ (907 mg, 4.27 mmol) was added 1,4-dioxane (8 mL) and H₂O (1 mL). The mixture was sparged with N₂ for 5 minutes and then treated with Pd(Ph₃P)₄ (165 mg, 0.143 mmol). The resultant mixture was subjected to microwave irradiation at 100° C. for 1 h. The reaction mixture was cooled to room temperature, filtered through a pad of diatomaceous earth. The filtrate was diluted with water (15 mL) and extracted with dichloromethane (3×40 mL). The combined organic solvent extracts were washed with brine (10 mL), dried over anhydrous Na₂SO₄, filtered, and concentrated. The product was purified by FCC (eluent: petroleum ether:ethyl acetate=20:1 to 5:1) a yellow solid. This product was further purified by preparative HPLC using an HPLC column, such as a Boston Prime C18 150 mm×30 mm×5 μm column (eluent: 75% to 100%, CH₃CN and H₂O (with 0.05% NH₄OH+10 mM NH₄HCO₃)). The product was then suspended in water (5 mL) and the mixture was frozen by insertion into a −78° C. bath. The frozen mixture was then lyophilized to dryness to yield 3-(4-bromofuran-2-yl)-1-tosyl-1H-pyrrolo[2,3-b]pyridine (70 mg, 12%) as a white solid. LCMS (ESI): mass calcd. for C₁₈H₁₃BrN₂O₃S, 417.27; m/z found, 419.2 [M+H]⁺.

Step B. (R)-7-(3-(5-(1-Tosyl-1H-pyrrolo[2,3-b]pyridin-3-yl)furan-3-yl)phenyl)-6,7-dihydro-5H-pyrrolo[1,2-a]imidazol-7-ol. To a microwave tube containing 3-(4 bromofuran-2-yl)-1-tosyl-1H-pyrrolo[2,3-b]pyridine (70 mg, 0.17 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 66 mg, 0.20 mmol), and K₃PO₄ (68 mg, 0.50 mmol) was added 1,4-dioxane (4 mL), and H₂O (1 mL). The mixture was sparged with Ar for 5 minutes and then treated with Pd(dtbpf)Cl₂ (11 mg, 0.017 mmol). The mixture was sparged with Ar for another 5 minutes and subjected to microwave irradiation at 90° C. for 1 h. The contents were cooled to room temperature and concentrated to dryness. The product was purified by FCC (eluent: petroleum ether:ethyl acetate=10:1 to 0:1) to yield (R)-7-(3-(5-(1-tosyl-1H-pyrrolo[2,3-b]pyridin-3-yl)furan-3-yl)phenyl)-6,7-dihydro-5H-pyrrolo[1,2-a]imidazol-7-ol (80 mg, 89%) as a brown solid. LCMS (ESI): mass calcd. for C₃₀H₂₄N₄O₄S, 536.15; m/z found, 537.2 [M+H]⁺.

### Intermediate 63. (R,S)-4-(3-(4,4,5,5-Tetramethyl-1,3,2-dioxaborolan-2-yl)phenyl)-5,6-dihydro-4H-cyclopenta[d]thiazol-4-ol

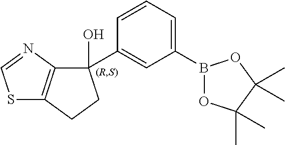

Step A. (E)-Ethyl 5-(3-ethoxy-3-oxoprop-1-en-1-yl)thiazole-4-carboxylate. (E) ethyl 3-(4,4,5,5-tetramethyl-1,3,2-dioxaborolan-2-yl)acrylate (11.5 g, 50.9 mmol) was added to a mixture of ethyl 5-bromothiazole-4-carboxylate (10.0 g, 42.4 mmol), K₂CO₃ (14.6 g, 106 mmol), 1,4-dioxane (80 mL), and H₂O (20 mL). The resultant mixture was sparged with N₂ for 2 min, treated with Pd(dppf)Cl₂ (3.10 g, 4.24 mmol), sparged with N₂ for another 2 min, and then heated at 80° C. for 16 h. After this time, the mixture was concentrated to dryness, and purified by FCC (eluent: petroleum ether ethyl acetate=1:0 to 1:1) to yield (E)-ethyl 5-(3-ethoxy-3-oxoprop-1-en-1-yl)thiazole-4-carboxylate (10 g, 75%) as a red oil. LCMS (ESI): mass calcd. for C₁₁H₁₃NO₄S, 255.06; m/z found, 256.0 [M+H]⁺.

Step B. Ethyl 5-(3-ethoxy-3-oxopropyl)thiazole-4-carboxylate. (E)-Ethyl 5-(3 ethoxy-3-oxoprop-1-en-1-yl)thiazole-4-carboxylate (10 g, 39 mmol), THF (200 mL) and wet Pd/C (5.0 g, 5 wt. %, 2.3 mmol) were added to a 1000 mL hydrogenation bottle. The suspension was degassed under vacuum and sparged with H₂ several times. The mixture was stirred under H₂ (15 Psi) at room temperature for 1 h. The suspension was filtered through a pad of diatomaceous earth and the pad washed with ethyl acetate (50 mL×2). The filtrate was concentrated to dryness under reduced pressure to yield ethyl 5-(3-ethoxy-3-oxopropyl)thiazole-4-carboxylate (10 g, 94%) as a yellow oil. LCMS (ESI): mass calcd. for C₁₁H₁₅NO₄S, 257.01; m/z found, 258.1 [M+H]⁺.

Step C. Ethyl 4-oxo-5,6-dihydro-4H-cyclopenta[d]thiazole-5-carboxylate. Ethyl 5-(3-ethoxy-3-oxopropyl)thiazole-4-carboxylate (1.0 g, 3.9 mmol) and t-BuOK (10 mL, 1.0 M in THF, 10 mmol) were added to a microwave tube. The resultant mixture was subjected to microwave irradiation at 100° C. for 1 h. After cooling to room temperature, the mixture was concentrated to dryness and purified by FCC (eluent: methylene chloride:MeOH=1:0 to 5:1) to yield ethyl 4-oxo-5,6-dihydro-4H-cyclopenta[d]thiazole-5-carboxylate (0.22 g, 71% purity, 95% yield) as a red oil. LCMS (ESI), mass calcd. for C₉H₉NO₃S, 211.03; m/z found, 212.1 [M+H]⁺.

Step D. 5,6-Dihydro-4H-cyclopenta[d]thiazol-4-one. Ethyl 4-oxo-5,6-dihydro-4H-cyclopenta[d]thiazole-5-carboxylate (1.0 g, 4.7 mmol), LiCl (422 mg, 9.95 mmol), water (0.10 mL, 5.6 mmol), and DMF (10 mL) were added to a microwave tube. The resultant mixture was subjected to microwave irradiation at 120° C. for 3 h. After cooling to room temperature, the mixture was concentrated to dryness and was purified by FCC (eluent: methylene chloride:MeOH=1:0 to 5:1) to yield 5,6-dihydro-4H-cyclopenta[d]thiazol-4-one (300 mg, 41%) as a red solid. LCMS (ESI), mass calcd. for C₆OH₅NOS, 139.01; m/z found, 140.1 [M+H]⁺.

Step E. (R,S)-4-(3-Bromophenyl)-5,6-dihydro-4H-cyclopenta[d]thiazol-4-ol. A flask containing 1,3-dibromobenzene (2.44 g, 10.3 mmol) and anhydrous THE (15 mL) was cooled to −72° C., and n-butyllithium (3.8 mL, 2.5 M in hexane, 9.5 mmol) was added dropwise. The mixture was stirred at −72° C. for 30 min, and then 5,6-dihydro-4H-cyclopenta[d]thiazol-4-one (1.2 g, 8.6 mmol) was added. The reaction was stirred at ⁻72° C. for 60 min. After this time, the mixture was quenched with aqueous saturated NH₄Cl solution (50 mL) and extracted with ethyl acetate (80 mL×3). The combined organic solvent extracts were dried over anhydrous Na₂SO₄, filtered, and concentrated to yield the product which was purified by FCC (eluent: petroleum ether:ethyl acetate, 1:0 to 0:1) to yield (R,S)-4-(3-bromophenyl)-5,6-dihydro-4H-cyclopenta[d]thiazol-4-ol (1.1 g, 44%) as a white solid. LCMS (ESI): mass calcd. for C₁₂H₁₀BrNOS, 296.18; m/z found, 298.0 [M+H]⁺.

Step F. (R,S)-4-(3-(4,4,5,5-Tetramethyl-1,3,2-dioxaborolan-2-yl)phenyl)-5,6-dihydro-4H-cyclopenta[d]thiazol-4-ol. [1,1′Bis(diphenylphosphino)ferrocene]dichloropalladium(II) (272 mg, 0.372 mmol) was added to a mixture of (R,S)-4-(3-bromophenyl)-5,6-dihydro-4H-cyclopenta[d]thiazol-4-ol (1.1 g, 3.7 mmol), 4,4,4′,4′,5,5,5′,5′-octamethyl-2,2′-bi(1,3,2-dioxaborolane) (1.89 g, 7.44 mmol), KOAc (1.09 g, 11.1 mmol), and 1,4-dioxane (30 mL). The resultant mixture was heated at 100° C. for 16 h under a N₂ atmosphere. After this time, the suspension was filtered through a pad of diatomaceous earth and the pad washed with ethyl acetate (50 mL×2). The filtrate was concentrated to dryness and the product, (R,S)-4-(3-(4,4,5,5-tetramethyl-1,3,2-dioxaborolan-2-yl)phenyl)-5,6-dihydro-4H-cyclopenta[d]thiazol-4-ol, (3.0 g) as a black solid. LCMS (ESI): mass calcd. for C₁₈H₂₂BNO₃S, 343.14; m/z found, 344.2 [M+H]⁺.

Step G. (R)-4-(3-(4,4,5,5-Tetramethyl-1,3,2-dioxaborolan-2-yl)phenyl)-5,6-dihydro-4H-cyclopenta[d]thiazol-4-ol and (S)-4-(3-(4,4,5,5-tetramethyl-1,3,2-dioxaborolan-2-yl)phenyl)-5,6-dihydro-4H-cyclopenta[d]thiazol-4-ol. The (R) and (S) enantiomers of (R,S)-4-(3-(4,4,5,5-tetramethyl-1,3,2-dioxaborolan-2-yl)phenyl)-5,6-dihydro-4H-cyclopenta[d]thiazol-4-ol were separated by preparative HPLC using an HPLC column such as a YMC Cellulose-SB, 250*50 mm, 5 μm; eluent: mobile phase A: Hexane; mobile phase B: IPA; Flow rate: 80 mL/Min; Gradient: 5% B for 25 min to afford the first eluting enantiomer (R)-4-(3-(4,4,5,5-tetramethyl-1,3,2-dioxaborolan-2-yl)phenyl)-5,6-dihydro-4H-cyclopenta[d]thiazol-4-ol (Intermediate 103), as a white solid. SFC Rt=2.95 min. LCMS (ESI): mass calcd. for C₁₂H₁₀BrNOS, 294.97; m/z found, 296 [M+H]⁺. ¹H-NMR (300 MHz, DMSO-d₆, ppm): δ 8.97 (s, 1H), 7.57-7.50 (m, 1H), 7.46-7.40 (m, 1H), 7.34-7.22 (m, 2H), 5.98 (s, 1H), 3.16-3.01 (m, 1H), 3.01-2.87 (m, 1H), 2.86-2.66 (m, 2H).

Also obtained from Step G was the second eluting enantiomer, (S) 4-(3-(4,4,5,5-tetramethyl-1,3,2-dioxaborolan-2-yl)phenyl)-5,6-dihydro-4H-cyclopenta[d]thiazol-4-ol, as a white solid. SFC Rt=3.52 min. LCMS (ESI): mass calcd. for C₁₂H₁₀BrNOS, 294.97; m/z found, 296 [M+H]⁺. ¹H-NMR (300 MHz, DMSO-d₆, ppm): δ 8.97 (s, 1H), 7.57-7.50 (m, 1H), 7.46-7.39 (m, 1H), 7.34-7.22 (m, 2H), 5.98 (s, 1H), 3.16-3.01 (m, 1H), 3.01-2.87 (m, 1H), 2.86-2.66 (m, 2H).

### Intermediate 64. (R,S)-4-(3-(2-(1-(phenylsulfonyl)-1H-pyrrolo[2,3-b]pyridin-3-yl)thiazol-4-yl)phenyl)-5,6-dihydro-4H-cyclopenta[d]thiazol-4-ol

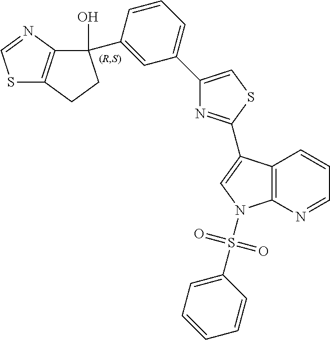

To a flask containing 4-bromo-2-(1-(phenylsulfonyl)-1H-pyrrolo[2,3-b]pyridin-3-yl)thiazole (Intermediate 2, 2.0 g, 4.8 mmol), (R,S)-4-(3-(4,4,5,5-tetramethyl-1,3,2-dioxaborolan-2-yl)phenyl)-5,6-dihydro-4H-cyclopenta[d]thiazol-4-ol (Intermediate 63, 3.0 g, 8.7 mmol), and K₃PO₄ (3.0 g, 14 mmol) was added 1,4-dioxane (40 mL) and H₂O (10 mL). The mixture was sparged with Ar for 5 minutes and then treated with Pd(dtbpf)Cl₂ (310 mg, 0.476 mmol). The mixture was sparged with Ar for another 5 minutes and then heated at 100° C. for 16 h. The mixture was cooled to room temperature, concentrated to dryness, and purified by FCC (eluent: petroleum ether:ethyl acetate=10:1 to 0:1, then ethyl acetate:MeOH=5:1) to yield 4-(3-(2-(1-(phenylsulfonyl)-1H-pyrrolo[2,3-b]pyridin-3-yl)thiazol-4-yl)phenyl)-5,6-dihydro-4H-cyclopenta[d]thiazol-4-ol (1.19 g, 65% purity, 29% yield) as a red oil. LCMS (ESI): mass calcd. for C₂₈H₂₀N₄O₃S₃, 556.07; m/z found, 557.0 [M+H]⁺.

### Intermediate 65. (R,S)-7-(3-(4,4,5,5-Tetramethyl-1,3,2-dioxaborolan-2-yl)phenyl)-7H-cyclopenta[b]pyridin-7-ol

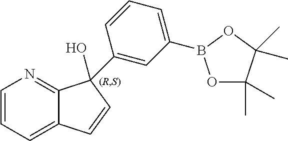 [SMILES: CC1(C)OB(c2cccc(C3(O)C=Cc4cccnc43)c2)OC1(C)C]

Step A. (R,S)-7-(3-Bromophenyl)-6,7-dihydro-5H-cyclopenta[b]pyridin-7-ol. A flask containing 1,3-dibromobenzene (10.6 g, 45.1 mmol) and anhydrous THE (50 mL) was cooled to −72° C. and then n-butyllithium (16.5 mL, 2.5 M in hexane, 41.3 mmol) was added dropwise. The resultant mixture was stirred at −72° C. for 30 min and then a solution of 5H-cyclopenta[b]pyridin-7(6H)-one (5.0 g, 37 mmol) in anhydrous THF (100 mL) was added dropwise and the resultant mixture was stirred at −72° C. for 30 minutes. After this time, the mixture was diluted with water (100 mL) and extracted with ethyl acetate (100 mL×3). The combined organic solvent extracts were washed with brine (150 mL), dried over anhydrous Na₂SO₄, filtered, and concentrated to yield the product which was purified by FCC (petroleum ether:ethyl acetate=1:0 to 1:1) to yield (R,S)-7-(3-bromophenyl)-6,7-dihydro-5H-cyclopenta[b]pyridin-7-ol product (5.2 g, 30%) as a brown oil. LCMS (ESI): mass calcd. for C₁₄H₁₂BrNO, 290.16; m/z found, 292.0 [M+H]⁺.

Step B. (R,S)-5-Bromo-7-(3-bromophenyl)-6,7-dihydro-5H-cyclopenta[b]pyridin-7-ol. 1-Bromopyrrolidine-2,5-dione (4.5 g, 25 mmol) was added to a mixture of 7-(3 bromophenyl)-6,7-dihydro-5H-cyclopenta[b]pyridin-7-ol (7.0 g, 24 mmol), benzoic peroxyanhydride (1.2 g, 4.8 mmol), and ACN (30 mL) at room temperature. The reaction mixture was heated to 90° C. for 2 h. After this time, the mixture was cooled to room temperature and concentrated under reduced pressure to afford the product, which was purified by FCC (eluent: petroleum ether:ethyl acetate=1:0 to 2:1) to afford (R,S)-5-Bromo-7-(3-bromophenyl)-6,7-dihydro-5H-cyclopenta[b]pyridin-7-ol (6.0 g, 81% purity, 55% Yield) as a red solid. LCMS (ESI): mass calcd. for C₁₄H₁₁Br₂NO, 369.05; m/z found, 369.9 [M+H]⁺.

Step C. (R,S)-7-(3-Bromophenyl)-7H-cyclopenta[b]pyridin-7-ol. 2,3,4,6,7,8,9,10-Octahydropyrimido[1,2-a]azepine (4.13 g, 27.1 mmol) was added to a mixture of 5-bromo-7-(3-bromophenyl)-6,7-dihydro-5H-cyclopenta[b]pyridin-7-ol, (Intermediate 1, Step B, 5.00 g, 13.5 mmol) and THF (50 mL). The resultant mixture was heated at 50° C. for 16 h. After this time, the mixture was diluted with water (50 mL) and extracted with ethyl acetate (50 mL×3). The combined organic solvent extracts were washed with brine (70 mL), dried over anhydrous Na₂SO₄, filtered, and concentrated to yield the product, which was purified by FCC (eluent: petroleum ether:ethyl acetate=1:0 to 1:1) to yield 7-(3-Bromophenyl)-7H-cyclopenta[b]pyridin-7-ol (1.5 g, 30%) as a brown oil. LCMS (ESI): mass calcd. for C₁₄H₁₀BrNO, 288.14; m/z found, 290.0 [M+H]⁺.

Step D. (R,S)-7-(3-(4,4,5,5-Tetramethyl-1,3,2-dioxaborolan-2-yl)phenyl)-7H-cyclopenta[b]pyridin-7-ol. [1,1′-Bis(diphenylphosphino)ferrocene]dichloropalladium(II) (381 mg, 0.521 mmol) was added to a mixture of 7-(3-bromophenyl)-7H-cyclopenta[b]pyridin-7-ol (1.50 g, 5.21 mmol), 4,4,4′,4′,5,5,5′,5′-octamethyl-2,2′-bi(1,3,2-dioxaborolane) (1.59 g, 6.25 mmol), KOAc (1.02 g, 10.4 mmol), and 1,4-dioxane (20 mL). The resultant mixture was heated at 90° C. under a N₂ atmosphere for 1 h. After this time, the mixture was filtered though a pad of diatomaceous earth and the pad washed with ethyl acetate (50 mL). The filtrate was diluted with water (50 mL) and extracted with ethyl acetate (50 mL×3). The combined organic solvent extracts were washed with brine, dried with anhydrous Na₂SO₄, filtered, and concentrated to yield the product, which was further purified by FCC (eluent: petroleum ether:ethyl acetate=1:0 to 1:1) to yield (R,S)-7-(3-(4,4,5,5-tetramethyl-1,3,2-dioxaborolan-2-yl)phenyl)-7H-cyclopenta[b]pyridin-7-ol (1.45 g, 72%) as a brown oil. LCMS (ESI): mass calcd. for C₂₀H₂₂BNO₃, 335.2; m/z found, 336.2 [M+H]⁺.

### Intermediate 66. (R,S)-7-(3-(2-(1-(Phenylsulfonyl)-1H-pyrrolo[2,3-b]pyridin-3-yl)thiazol-4-yl)phenyl)-7H-cyclopenta[b]pyridin-7-ol

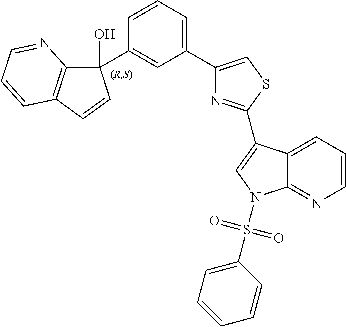

(R,S)-7-(3-(4,4,5,5-Tetramethyl-1,3,2-dioxaborolan-2-yl)phenyl)-7H-cyclopenta[b]pyridin-7-ol (Intermediate 65, 1.30 g, 3.88 mmol), 4-bromo-2-(1-(phenylsulfonyl)-1H-pyrrolo[2,3-b]pyridin-3-yl)thiazole (Intermediate 2, 1.96 g, 4.65 mmol), K₃PO₄ (2.47 g, 11.6 mmol), and 1,4-dioxane/H₂O (13 mL, 4:1) were added to a microwave tube. The mixture was sparged with N₂ for 5 minutes. Pd(dtbpf)Cl₂ (253 mg, 0.388 mmol) was added and the mixture sparged with N₂ for another 5 minutes. The resultant mixture was subjected to microwave irradiation at 100° C. for 2 h. After this time, the reaction mixture was filtered through a pad of diatomaceous earth and the pad washed with ethyl acetate (30 mL). The filtrate was diluted with water (30 mL) and extracted with ethyl acetate (3×30 mL). The combined organic solvent extracts were washed with brine, dried over anhydrous Na₂SO₄, filtered, and concentrated to yield (R,S)-7-(3-(2-(1-(phenylsulfonyl)-1H-pyrrolo[2,3-b]pyridin-3-yl)thiazol-4-yl)phenyl)-7H-cyclopenta[b]pyridin-7-ol (2.13 g) as a brown oil, which was used without further purification. LCMS (ESI): mass calcd. for C₃₀H₂₀N₄O₃S₂, 548.6; m/z found, 550.2 [M+H]⁺.

### Intermediate 67. (R,S)-6,6-Dimethyl-7-(3-(4,4,5,5-tetramethyl-1,3,2-dioxaborolan-2-yl)phenyl)-6,7-dihydro-5H-cyclopenta[b]pyridin-7-ol

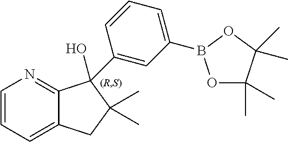 [SMILES: CC1(C)Cc2cccnc2C1(O)c1cccc(B2OC(C)(C)C(C)(C)O2)c1]

Step A. 6,6-Dimethyl-5H-cyclopenta[b]pyridin-7(6H)-one. A solution of 5H-cyclopenta[b]pyridin-7(6H)-one (4.7 g, 35 mmol), iodomethane (18.1 g, 127 mmol), and THF (100 mL) was cooled to 0° C.; and then potassium tert-butoxide (80 mL, 1.0 M in THF, 80 mmol) was added. The resulting mixture was stirred at 0° C. for 4 h. After this time, the reaction mixture was quenched with water (100 mL) and extracted with ethyl acetate (50 mL×3). The combined organic solvent extracts were washed with brine, dried over anhydrous Na₂SO₄, filtrated, and concentrated to yield the product, which was purified by FCC (eluent: petroleum ether:ethyl acetate, 1:0 to 3:7) to yield 6,6-dimethyl-5H-cyclopenta[b]pyridin-7(6H)-one (2.6 g, 44%) as a colorless oil. LCMS (ESI): mass calcd. for C₁₀H₁₁NO, 161.1; m/z found, 161.9 [M+H]⁺.

Step B. 7-(3-Bromophenyl)-6,6-dimethyl-6,7-dihydro-5H-cyclopenta[b]pyridin-7-ol. A solution of 1,3-dibromobenzene (2.00 mL, 16.5 mmol) and anhydrous THF (50 mL) was cooled to −72° C. and then n-Butyllithium (7.00 mL, 2.5 M in hexane, 17.5 mmol) was added. The resultant mixture was stirred at −72° C. for 30 min, followed the addition of 6,6-dimethyl-5H-cyclopenta[b]pyridin-7(6H)-one (2.60 g, 15.6 mmol) in anhydrous THF (10 mL). The mixture was stirred at −72° C. for another 30 minutes. After this time, the reaction mixture was quenched with aqueous saturated NH₄Cl solution (50 mL), diluted with water (50 mL), and extracted with ethyl acetate (80 mL×3). The combined organic solvent extracts were washed with brine, dried over anhydrous Na₂SO₄, filtrated, and concentrated to yield the product, which was purified by FCC (eluent: petroleum ether:ethyl acetate, 1:0 to 3:2) to yield 7-(3-bromophenyl)-6,6-dimethyl-6,7-dihydro-5H-cyclopenta[b]pyridin-7-ol (3.12 g, 94% purity, 59%) as a yellow oil. LCMS (ESI): mass calcd. for C₁₆H₁₆BrNO, 317.04; m/z found, 318.0 [M+H]⁺.

Step C. (R,S)-6,6-Dimethyl-7-(3-(4,4,5,5-tetramethyl-1,3,2-dioxaborolan-2-yl)phenyl)-6,7-dihydro-5H-cyclopenta[b]pyridin-7-ol. [1,1′-bis(diphenylphosphino)ferrocene]dichloropalladium(II) (775 mg, 0.1.06 mmol) was added to a mixture of 7-(3-bromophenyl)-6,6-dimethyl-6,7-dihydro-5H-cyclopenta[b]pyridin-7-ol (3.1 g, 9.7 mmol), 4,4,4′,4′,5,5,5′,5′-octamethyl-2,2′-bi(1,3,2-dioxaborolane) (5.0 g, 20 mmol), KOAc (3.0 g, 31 mmol), and 1,4-dioxane (40 mL). The resultant mixture was sparged with N₂ for 5 minutes and then heated at 80° C. under a N₂ atmosphere for 2 h. After cooling to room temperature, the reaction mixture was filtered through a pad of diatomaceous earth and the pad washed with ethyl acetate (50 mL). The filtrate was diluted with water (50 mL) and extracted with ethyl acetate (50 mL×3). The combined organic solvent extracts were dried over anhydrous Na₂SO₄, filtered, concentrated, and purified by FCC (eluent: petroleum ether:ethyl acetate, 1:0 to 1:1) to yield (R,S)-6,6-dimethyl-7-(3-(4,4,5,5-tetramethyl-1,3,2-dioxaborolan-2-yl)phenyl)-6,7-dihydro-5H-cyclopenta[b]pyridin-7-ol (3.4 g, 64% purity, 61%) as a yellow oil. LCMS (ESI): mass calcd. for C₂₂H₂₈BNO₃, 365.22; m/z found, 366.2 [M+H]⁺.

### Intermediate 68. (R,S)-6,6-dimethyl-7-(3-(2-(1-(phenylsulfonyl)-1H-pyrrolo[2,3-b]pyridin-3-yl)thiazol-4-yl)phenyl)-6,7-dihydro-5H-cyclopenta[b]pyridin-7-ol

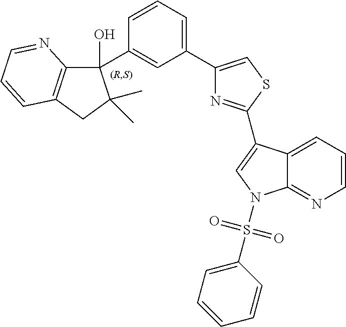

To a microwave tube containing (R,S)-6,6-dimethyl-7-(3-(4,4,5,5-tetramethyl-1,3,2-dioxaborolan-2-yl)phenyl)-6,7-dihydro-5H-cyclopenta[b]pyridin-7-ol (Intermediate 67, 1.0 g, 1.7 mmol), 4-bromo-2-(1-(phenylsulfonyl)-1H-pyrrolo[2,3-b]pyridin-3-yl)thiazole (Intermediate 2, 740 mg, 1.76 mmol) was added 1,4-dioxane/H₂O (4:1, 10 mL). The mixture was sparged with N₂ for 5 minutes followed by the addition of Pd(dtbpf)Cl₂ (140 mg, 0.215 mmol) and K₃PO₄ (1.1 g, 5.2 mmol). The mixture was sparged with N₂ for another 5 minutes and subjected to microwave irradiation at 100° C. for 2 h. After this time, the mixture was cooled to room temperature, filtered through a pad of diatomaceous earth and the pad washed with ethyl acetate (30 mL). The filtrate was diluted with water (20 mL) and extracted with ethyl acetate (20 mL×3). The combined organic solvent extracts were washed with brine, dried over anhydrous Na₂SO₄, filtered, and concentrated yield (R,S)-6,6-dimethyl-7-(3-(2-(1-(phenylsulfonyl)-1H-pyrrolo[2,3-b]pyridin-3-yl)thiazol-4-yl)phenyl)-6,7-dihydro-5H-cyclopenta[b]pyridin-7-ol, as a red solid, which was used to next step without further purification. LCMS (ESI): mass calcd. for C₃₂H₂₈N₄O₃S₂, 578.14; m/z found, 579.2 [M+H]⁺.

### Intermediate 69. (R,S)-8-(3-(4,4,5,5-Tetramethyl-1,3,2-dioxaborolan-2-yl)phenyl)-5,6,7,8-tetrahydroimidazo[1,2-a]pyridin-8-ol

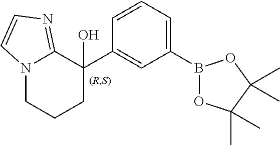 [SMILES: CC1(C)OB(c2cccc(C3(O)CCCn4ccnc43)c2)OC1(C)C]

Step A. 6,7-Dihydroimidazo[1,2-a]pyridin-8(5H)-one. Manganese(IV) oxide (8.18 g, 94.1 mmol) was added in portions to a solution of 5,6,7,8-tetrahydroimidazo[1,2-a]pyridin-8-ol (1300 mg, 9.409 mmol) and methylene chloride (30 mL). The resultant mixture was stirred at room temperature for 16 h. After this time, the suspension was filtered through a pad of diatomaceous earth and the pad washed with methylene chloride (20 mL×2). The filtrate was concentrated to yield 6,7-dihydroimidazo[1,2-a]pyridin-8(5H)-one (1.0 g, 78%) as a white solid. ¹H NMR (400 MHz, CDCl₃) δ 7.33 (s, 1H), 7.05 (s, 1H), 4.30-4.18 (m, 2H), 2.80-2.72 (m, 2H), 2.42-2.31 (m, 2H).

Step B. (R,S)-8-(3-Bromophenyl)-5,6,7,8-tetrahydroimidazo[1,2-a]pyridin-8-ol. A solution of 1,3-dibromobenzene (1.66 g, 7.05 mmol) and anhydrous THE (10 mL) was cooled to −72° C. and n-butyllithium (2.4 mL, 6.0 mmol, 2.5 M in hexane) was added dropwise. The resultant mixture was stirred at −72° C. for 30 min and then treated with 6,7-dihydroimidazo[1,2-a]pyridin-8(5H)-one (800 mg, 5.88 mmol). The resultant mixture was stirred at −72° C. for 60 min. After this time, the reaction was quenched with aqueous saturated NH₄Cl solution (50 mL) and extracted with ethyl acetate (80 mL×3). The combined organic solvent extracts were washed with brine, dried over anhydrous Na₂SO₄, filtrated, and concentrated to yield the product, which was purified by FCC (eluent: petroleum ether:ethyl acetate, 1:0 to 0:1) to yield (R,S)-8-(3-bromophenyl)-5,6,7,8-tetrahydroimidazo[1,2-a]pyridin-8-ol (800 mg, 45%) as a white solid. LCMS (ESI): mass calcd. for C₁₃H₁₃BrN₂O, 292.02; m/z found, 293.1 [M+H]⁺.

Step C. (R,S)-8-(3-(4,4,5,5-Tetramethyl-1,3,2-dioxaborolan-2-yl)phenyl)-5,6,7,8-tetrahydroimidazo[1,2-a]pyridin-8-ol. To a tube containing [1,1′-Bis(diphenylphosphino)-ferrocene]palladium(II) dichloride (175 mg, 0.239 mmol), (R,S)-8-(3-bromophenyl)-5,6,7,8-tetrahydroimidazo[1,2-a]pyridin-8-ol (700 mg, 2.39 mmol), 4,4,4′,4′,5,5,5′,5′-octamethyl-2,2′-bi(1,3,2-dioxaborolane) (1.21 g, 4.78 mmol), and KOAc (703 mg, 7.16 mmol) was added 1,4-dioxane (25 mL). The resultant mixture was subjected to microwave irradiation at 100° C. for 60 min. After this time, the mixture was filtered through a pad of diatomaceous earth and the pad washed with ethyl acetate (50 mL×2). The filtrate was concentrated to dryness under reduced pressure to yield (R,S)-8-(3-(4,4,5,5-tetramethyl-1,3,2-dioxaborolan-2-yl)phenyl)-5,6,7,8-tetrahydroimidazo[1,2-a]pyridin-8-ol, (2.36 g) as a black solid, which was used in the next step without further purification. LCMS (ESI): mass calcd. for C₁₉H₂₅BN₂O₃, 340.20; m/z found, 341.2 [M+H]⁺.

### Intermediate 70. (R,S)-8-(3-(2-(1-(Phenylsulfonyl)-1H-pyrrolo[2,3-b]pyridin-3-yl)thiazol-4-yl)phenyl)-5,6,7,8-tetrahydroimidazo[1,2-a]pyridin-8-ol

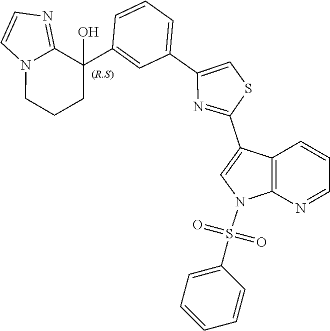 [SMILES: O=S(=O)(c1ccccc1)n1cc(-c2nc(-c3cccc(C4(O)CCCn5ccnc54)c3)cs2)c2cccnc21]

To a tube containing 1,1′-bis (di-t-butylphosphino)ferrocene palladium dichloride (248 mg, 0.381 mmol), 4-bromo-2-(1-(phenylsulfonyl)-1H-pyrrolo[2,3-b]pyridin-3-yl)thiazole (Intermediate 2, 1.6 g, 3.8 mmol), (R,S)-8-(3-(4,4,5,5-tetramethyl-1,3,2-dioxaborolan-2-yl)phenyl)-5,6,7,8-tetrahydroimidazo[1,2-a]pyridin-8-ol (Intermediate 69, 1.94 g, 5.7 mmol), and K₃PO₄ (2.42 g, 11.4 mmol) was added 1,4-dioxane (16 mL), and H₂O (4 mL). The resultant mixture was subjected to microwave irradiation at 100° C. for 16 h. After this time, the mixture was concentrated to dryness to yield the product, which was purified by FCC (with an initial eluent: petroleum ether:ethyl acetate, 10:1 to 0:1, and then a second eluent of ethyl acetate:CH₃OH, 5:1), and then triturated with petroleum ether/ethyl acetate (1:10, 40 mL) to yield (R,S)-8-(3-(2-(1-(phenylsulfonyl)-1H-pyrrolo[2,3-b]pyridin-3-yl)thiazol-4-yl)phenyl)-5,6,7,8-tetrahydroimidazo[1,2-a]pyridin-8-ol (910 mg, 38% yield) as a gray solid. LCMS (ESI): mass calcd. for C₂₉H₂₃N₅O₃S₂, 553.12; m/z found, 554.2 [M+H]⁺.

### Intermediate 71. (R,S)-3-Hydroxy-1-methyl-3-(3-(2-(5-tosyl-5H-pyrrolo[2,3-b]pyrazin-7-yl)thiazol-4-yl)phenyl)pyrrolidin-2-one

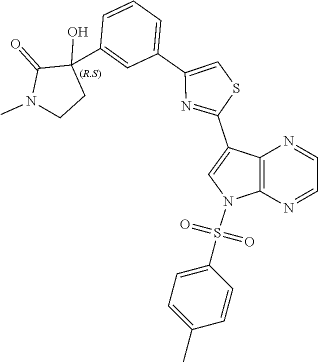 [SMILES: Cc1ccc(S(=O)(=O)n2cc(-c3nc(-c4cccc(C5(O)CCN(C)C5=O)c4)cs3)c3nccnc32)cc1]

To a microwave tube containing 4-bromo-2-(5-tosyl-5H-pyrrolo[2,3-b]pyrazin-7-yl)thiazole (Intermediate 10, 550 mg, 1.3 mmol), (R,S)-3-hydroxy-1-methyl-3-(3-(4,4,5,5-tetramethyl-1,3,2-dioxaborolan-2-yl)phenyl)pyrrolidin-2-one (Intermediate 4, 481 mg, 1.52 mmol), and K₃PO₄ (805 mg, 3.79 mmol) was added 1,4-dioxane (10 mL), and H₂O (2 mL). The mixture was sparged with Ar for 5 minutes and then Pd(dtbpf)Cl₂ (82 mg, 0.13 mmol) was added. The mixture was sparged with Ar for another 5 minutes and subjected to microwave irradiation at 80° C. for 1 h. The reaction mixture was cooled to room temperature, diluted with water (60 mL), and extracted with ethyl acetate (50 mL×3). The combined organic solvent extracts were dried over anhydrous Na₂SO₄, filtered, and concentrated to yield the product which was purified by preparative HPLC using an HPLC column, such as a Boston Green ODS C18, 150×40 mm×10 μm column (eluent: 51% to 76%, CH₃CN and H₂O (with 10 mM NH₄HCO₃)) to yield pure product. The product was suspended in water (10 mL) and the mixture was frozen by insertion into a −78° C. bath. The frozen mixture was then lyophilized to yield (R,S)-3-hydroxy-1-methyl-3-(3-(2-(5-tosyl-5H-pyrrolo[2,3-b]pyrazin-7-yl)thiazol-4-yl)phenyl)pyrrolidin-2-one (370 mg, 54%) as a brown solid. ¹H NMR (400 MHz, DMSO-d₆) δ 8.85 (s, 1H), 8.80 (d, J=2.4 Hz, 1H), 8.59 (d, J=2.4 Hz, 1H), 8.23 (s, 1H), 8.18-8.11 (m, 3H), 8.00 (d, J=7.6 Hz, 1H), 7.50-7.42 (m, 3H), 7.35 (d, J=7.8 Hz, 1H), 6.14 (s, 1H), 3.53-3.37 (m, 2H), 2.88 (s, 3H), 2.43-2.26 (m, 5H).

### Intermediate 72. Ethyl 3-((2,2,2-trifluoroethyl)amino)propanoate

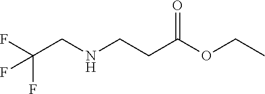

A flask containing 2,2,2-trifluoroethanamine (10.0 g, 101 mmol), ethyl acrylate (10.1 g, 101 mmol), and 1,1,1,3,3,3-hexafluoro-2-propanol (50 mL) was heated at 110° C. for 16 h. The reaction mixture was cooled to room temperature and the mixture was combined with another batch of material. The combined mixture was concentrated to dryness, dissolved in water (150 mL), of the pH was adjusted to pH=2-3 with 1 N HCl, and washed with ethyl acetate (100 mL×2). The combined organic solvent extracts were concentrated to dryness under reduced pressure. The resulting oil was re-dissolved in ethyl acetate (300 mL) and the pH was adjusted to pH=8-9 with concentrated NH₃. The mixture was directly dried over anhydrous Na₂SO₄, filtered, and concentrated to yield ethyl 3-((2,2,2-trifluoroethyl)amino)-propanoate (17.0 g) as a yellow oil. LCMS(ESI), mass calcd. for C₇H₁₂F₃NO₂, 199.08; m/z found, 200.2 [M+H]⁺. ¹H NMR (400 MHz, DMSO-d₆) S 4.05 (q, J=7.1 Hz, 2H), 3.22 (q, J=10.3 Hz, 2H), 2.83 (t, J=6.8 Hz, 2H), 2.42 (t, J=6.8 Hz, 2H), 1.17 (t, J=7.1 Hz, 3H).

### Intermediate 73: Ethyl 4,5-dioxo-1-(2,2,2-trifluoroethyl)pyrrolidine-3-carboxylate

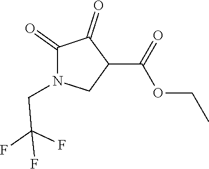 [SMILES: CCOC(=O)C1CN(CC(F)(F)F)C(=O)C1=O]

To a flask containing ethyl 3-((2,2,2-trifluoroethyl)amino)propanoate (Intermediate 72, 17 g, 85 mmol), diethyl oxalate (13.5 g, 92.4 mmol), and THE (150 mL) was added t-BuOK (12.5 g, 111 mmol). The resultant mixture was heated at 75° C. for 5 h. This reaction mixture was cooled to room temperature and the pH was adjusted to pH=2 with 2 N HCl. The solvent was removed under reduced pressure and the precipitate was filtered, washed with water (50 mL) and dried under vacuum. The resulting residue was triturated with petroleum ether (100 mL) and the suspension isolated via filtration. The filter cake was dried under reduced pressure to yield ethyl 4,5-dioxo-1-(2,2,2-trifluoroethyl)pyrrolidine-3-carboxylate (16 g, 74%) as a brown solid. LCMS (ESI): mass calcd. for C₉H₁₀F₃NO₄, 253.06; m/z found, 254.1 [M+H]⁺.

### Intermediate 74. 1-(2,2,2-Trifluoroethyl)pyrrolidine-2,3-dione

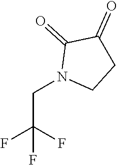 [SMILES: O=C1CCN(CC(F)(F)F)C1=O]

To ethyl 4,5-dioxo-1-(2,2,2-trifluoroethyl)pyrrolidine-3-carboxylate (Intermediate 73, 10 g, 39 mmol) was added aqueous HCl (3 M, 250 mL) and the resultant mixture was heated at 100° C. for 5 h. The reaction mixture was cooled to room temperature, concentrated to dryness, and purified by FCC (eluent: petroleum ether:ethyl acetate (containing 0.5% HCOOH)=5:1 to 0:1) to yield 1-(2,2,2-trifluoroethyl)pyrrolidine-2,3-dione (2.5 g, 24%) as a brown solid. LCMS (ESI): mass calcd. for C₆H₆F₃NO₂, 181.04; m/z found, 182.1 [M+H]⁺.

### Intermediate 75. (R,S)-3-(3-Bromophenyl)-3-hydroxy-1-(2,2,2-trifluoroethyl)pyrrolidin-2-one

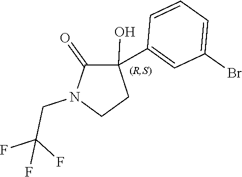

A solution of 1,3-dibromobenzene (2.4 g, 10 mmol) and anhydrous THE (20 mL) was cooled to −70° C. and n-BuLi (4.0 mL, 2.5 M in hexane, 10 mmol) was added dropwise. The resultant mixture was stirred at −70° C. for 30 min and then 1-(2,2,2-trifluoroethyl)pyrrolidine-2,3-dione (Intermediate 74, 1.3 g, 7.2 mmol) was added. The mixture was maintained at −50° C. for 30 min, then quenched with aqueous saturated NH₄Cl solution (30 mL), warmed to room temperature, and extracted with ethyl acetate (30 mL×3). The combined organic solvent extracts were dried over anhydrous Na₂SO₄, filtered, concentrated and purified by HPLC using an HPLC column, such as an Xtimate C18, 150 mm×40 mm×10 μm column (eluent: 44% to 64%, CH₃CN and H₂O (with 0.2% HCOOH)). The product was suspended in water (10 mL) and the mixture was frozen by insertion into a −78° C. bath. The frozen mixture was then lyophilized to yield (R,S)-3-(3-bromophenyl)-3-hydroxy-1-(2,2,2-trifluoroethyl)pyrrolidin-2-one (320 mg) as a white solid. LCMS (ESI): mass calcd. for C₁₂H₁₁BrF₃NO₂, 336.99; m/z found, 340.0 [M+H]⁺.

### Intermediate 76. (S)-3-(3-Bromophenyl)-3-hydroxy-1-(2,2,2-trifluoroethyl)pyrrolidin-2-one

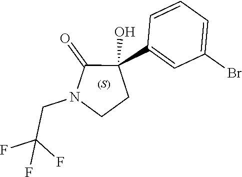

The enantiomers of (R,S)-3-(3-bromophenyl)-3-hydroxy-1-(2,2,2-trifluoroethyl)pyrrolidin-2-one (Intermediate 75, 320 mg, 0.95 mmol) were separated by SFC using an SFC column, such as a DAICEL CHIRALPAK AD-H 250 mm×30 mm, 5 μm column (isocratic elution: i-PrOH (containing 0.1% of 25% aqueous NH₃): supercritical CO₂, 20%: 80%). The first eluting enantiomer was (S)-3-(3-bromophenyl)-3-hydroxy-1-(2,2,2-trifluoroethyl)pyrrolidin-2-one and the second eluting enantiomer was (R)-3-(3-bromophenyl)-3-hydroxy-1-(2,2,2-trifluoroethyl)pyrrolidin-2-one (Intermediate 77). The fractions containing (S)-3-(3-bromophenyl)-3-hydroxy-1-(2,2,2-trifluoroethyl)pyrrolidin-2-one were collected and concentrated under reduced pressure. The product was suspended in water (10 mL) and the mixture was frozen by insertion into a −78° C. bath. The frozen mixture was then lyophilized to yield, (S)-3-(3-bromophenyl)-3-hydroxy-1-(2,2,2-trifluoroethyl)pyrrolidin-2-one, SFC Rₜ=1.174 min, (90 mg, 28%) as a white solid. LCMS (ESI) mass calcd. for C₁₂H₁₁BrF₃NO₂, 336.99; m/z found, 340.0 [M+H]⁺. ¹H NMR (400 MHz, DMSO-d₆) δ 7.53 (s, 1H), 7.49-7.44 (m, 1H), 7.30 (d, J=4.9 Hz, 2H), 6.36 (s, 1H), 4.25-4.02 (m, 2H), 3.55 (dt, J=8.8, 5.1 Hz, 1H), 3.46-3.36 (m, 1H), 2.39-2.18 (m, 2H).

### Intermediate 77. (R)-3-(3-Bromophenyl)-3-hydroxy-1-(2,2,2-trifluoroethyl)pyrrolidin-2-one

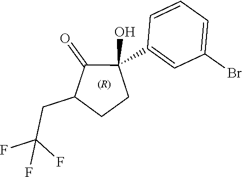

(R)-3-(3-bromophenyl)-3-hydroxy-1-(2,2,2-trifluoroethyl)pyrrolidin-2-one (100 mg, 31%) was obtained from the separation described in Intermediate 76, as a white solid. SFC Rₜ=2.122 min. LCMS (ESI): mass calcd. for C₁₂H₁₁BrF₃NO₂, 336.99; m/z found, 338.0 [M+H]⁺.

### Intermediate 78. (R)-3-Hydroxy-3-(3-(4,4,5,5-tetramethyl-1,3,2-dioxaborolan-2-yl)phenyl)-1-(2,2,2-trifluoroethyl)pyrrolidin-2-one

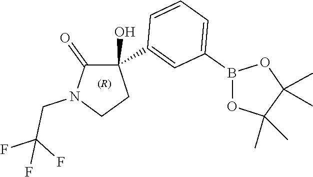

To a microwave tube containing (R)-3-(3-bromophenyl)-3-hydroxy-1-(2,2,2-trifluoroethyl)pyrrolidin-2-one (Intermediate 77, 100 mg, 0.296 mmol), 4,4,4′,4′,5,5,5′,5′-octamethyl-2,2′-bi(1,3,2-dioxaborolane) (150 mg, 0.591 mmol), and KOAc (87 mg, 0.89 mmol) was added 1,4-dioxane (10 mL). The mixture was sparged with N₂ for 5 minutes and then Pd(dppf)Cl₂ (22 mg, 0.030 mmol) was added. The mixture was heated at 90° C. in a microwave for 1 h. The reaction mixture was cooled to room temperature, filtered through a pad of diatomaceous earth and the pad washed with ethyl acetate (10 mL). The filtrate was concentrated to dryness and purified by FCC (eluent: petroleum ether:ethyl acetate=1:0 to 0:1) to yield (R)-3-hydroxy-3-(3-(4,4,5,5-tetramethyl-1,3,2-dioxaborolan-2-yl)phenyl)-1-(2,2,2-trifluoroethyl)pyrrolidin-2-one (120 mg, 80%) as a yellow oil. LCMS(ESI): mass calcd. for C₁₈H₂₃BF₃NO₄, 385.17; m/z found, 368.2 [M−18+H]⁺.

### Intermediate 79. (R)-3-Hydroxy-3-(3-(2-(5-tosyl-5H-pyrrolo[2,3-b]pyrazin-7-yl)thiazol-4-yl)phenyl)-1-(2,2,2-trifluoroethyl)pyrrolidin-2-one

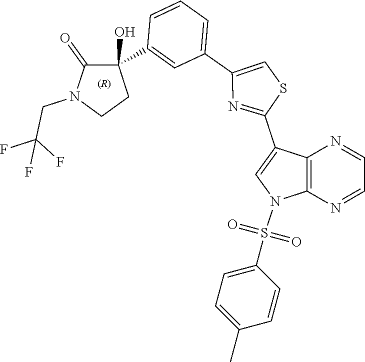

To a microwave tube containing 4-bromo-2-(5-tosyl-5H-pyrrolo[2,3-b]pyrazin-7-yl)thiazole (Intermediate 10, 140 mg, 0.322 mmol), (R)-3-hydroxy-3-(3-(4,4,5,5-tetramethyl-1,3,2-dioxaborolan-2-yl)phenyl)-1-(2,2,2-trifluoroethyl)pyrrolidin-2-one (Intermediate 78, 120 mg, 0.312 mmol), K₃PO₄ (198 mg, 0.933 mmol) was added 1,4-dioxane (12 mL), and H₂O (3 mL). The mixture was sparged with Ar for 5 minutes and then Pd(dtbpf)Cl₂ (20 mg, 0.031 mmol) was added. The mixture was sparged with Ar for another 5 minutes and then subjected to microwave irradiation at 80° C. for 1 h. The reaction mixture was cooled to room temperature, filtered through a pad of diatomaceous earth and the pad washed with ethyl acetate (10 mL). The filtrate was concentrated to dryness and purified by FCC (eluent: petroleum ether:ethyl acetate=1:0 to 0:1) to yield (R)-3-hydroxy-3-(3-(2-(5-tosyl-5H-pyrrolo[2,3-b]pyrazin-7-yl)thiazol-4-yl)phenyl)-1-(2,2,2-trifluoroethyl)pyrrolidin-2-one (150 mg, 76%) as a yellow solid. LCMS(ESI): mass calcd. for C₂₈H₂₂F₃N₅O₄S₂, 613.11; m/z found, 614.2 [M+H].

### Intermediate 80. Methyl 3-((tert-butyldimethylsilyl)oxy)benzoate

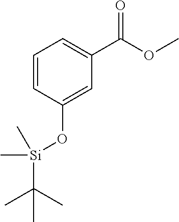

To a flask containing methyl 3-hydroxybenzoate (3.00 g, 19.7 mmol), ¹H-imidazole (4.03 g, 59.2 mmol) in dichloromethane (40 mL) was added TBSCl (3.86 g, 25.6 mmol). The mixture was stirred at room temperature for 1 h, then filtered and the filtrate was concentrated and purified by FCC (eluent: petroleum ether:ethyl acetate=1:0 to 10:1) to yield methyl 3-((tert-butyldimethylsilyl)oxy)benzoate (3.9 g, 74%) as a colorless oil. LCMS(ESI): mass calcd. for C₁₄H₂₂O₃Si, 266.13; m/z found, 267.1 [M+H]⁺. ¹H NMR (400 MHz, CDCl₃-d) δ 7.66-7.61 (m, 1H), 7.51-7.48 (m, 1H), 7.32-7.27 (m, 1H), 7.06-7.01 (m, 1H), 3.91 (s, 3H), 1.01-0.99 (m, 9H), 0.23-0.20 (m, 6H).

### Intermediate 81. (R,S)-3-(3-((tert-Butyldimethylsilyl)oxy)phenyl)-3-hydroxy-1-methylazetidine-2-thione

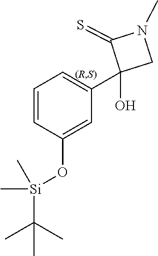

A solution of methyl 3-((tert-butyldimethylsilyl)oxy)benzoate (Intermediate 80, 6.10 g, 22.9 mmol), N,N-dimethylmethanethioamide (2.04 g, 22.9 mmol), and THF (30 mL) was added dropwise to a solution of LDA (27.5 mL, 2 M in hexane, 55.0 mmol) and THF (30 mL) that had been cooled to −70° C. The reaction mixture was stirred at −70° C. for 1 h, then slowly warmed to room temperature and quenched with water (50 mL). The resultant mixture was extracted with ethyl acetate (50 mL×3), and the combined organic solvent extracts were washed with brine, dried over anhydrous Na₂SO₄, filtered, and concentrated to dryness to yield the product which was purified by FCC (eluent: petroleum ether:ethyl acetate=1:0 to 5:1) to yield (R,S)-3-(3-((tert-butyldimethylsilyl)oxy)phenyl)-3-hydroxy-1-methylazetidine-2-thione (3.2 g, 43%) as a yellow solid. LCMS(ESI): mass calcd. for C₁₆H₂₅NO₂Ssi, 323.14; m/z found, 324.2 [M+H]⁺. ¹H NMR (400 MHz, CDCl₃) δ 7.26-7.22 (m, 1H), 7.14 (d, J=7.9 Hz, 1H), 7.11-7.07 (m, 1H), 6.84-6.80 (m, 1H), 4.12 (d, J=6.8 Hz, 1H), 4.00 (d, J=6.8 Hz, 1H), 3.24 (s, 3H), 0.99 (s, 9H), 0.21 (s, 6H).

### Intermediate 82. (R,S)-3-(3-((tert-Butyldimethylsilyl)oxy)phenyl)-3-hydroxy-1-methylazetidin-2-one

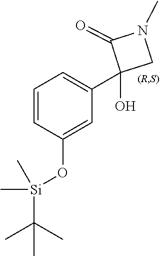

A solution of (R,S)-3-(3-((tert butyldimethylsilyl)oxy)phenyl)-3-hydroxy-1-methylazetidine-2-thione (Intermediate 81, 3.00 g, 9.27 mmol) and dichloromethane (70 mL) was cooled to −70° C. Then, O₃ (15 psi) was bubbled through the solution for 30 minutes (until the blue color persisted). The mixture was warmed to room temperature, and concentrated to dryness, and the product was purified by FCC (eluent: petroleum ether:ethyl acetate=1:0 to 3:1) to yield (R,S)-3-(3-((tert-butyldimethylsilyl)oxy)phenyl)-3-hydroxy-1-methylazetidin-2-one (1.1 g, 37%) as a yellow solid. LCMS(ESI): mass calcd. for C₁₆H₂₅NO₃Si, 307.16; m/z found, 308.2 [M+H].

### Intermediate 83. (R,S)-3-(3-((tert-Butyldimethylsilyl)oxy)phenyl)-1-methyl-2-oxoazetidin-3-yl acetate

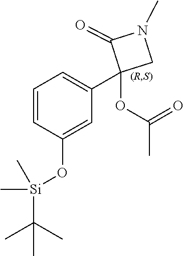 [SMILES: CC(=O)OC1(c2cccc(O[Si](C)(C)C(C)(C)C)c2)CN(C)C1=O]

To a flask containing (R,S)-3-(3-((tert-butyldimethylsilyl)oxy)phenyl)-3-hydroxy-1-methylazetidin-2-one (Intermediate 82, 1.1 g, 3.6 mmol), DIPEA (1.9 mL, 11 mmol), and dichloromethane (40 mL) was added acetyl chloride (11 mL, 50 mg/mL in dichloromethane, 7.0 mmol). The reaction mixture was stirred at room temperature for 2 h, diluted with H₂O (30 mL) and extracted with dichloromethane (30 mL×3). The combined organic solvent extracts were washed with brine, dried over anhydrous Na₂SO₄, filtered, and concentrated to yield (R,S)-3-(3-((tert-butyldimethylsilyl)oxy)phenyl)-1-methyl-2-oxoazetidin-3-yl acetate, (1.2 g) as a brown oil. LCMS(ESI): mass calcd. for C₁₈H₂₇NO₄Si, 349.17; m/z found, 290.1 [M-60+H]⁺. ¹H NMR (400 MHz, DMSO-d₆) δ 7.34-7.22 (m, 1H), 7.09 (d, J=8.3 Hz, 1H), 6.93-6.81 (m, 2H), 4.01 (d, J=6.8 Hz, 1H), 3.77 (d, J=6.8 Hz, 1H), 2.83 (s, 3H), 2.16-2.06 (m, 3H), 0.95 (s, 9H), 0.18 (s, 6H).

### Intermediate 84. (R,S)-3-(3-Hydroxyphenyl)-1-methyl-2-oxoazetidin-3-yl acetate

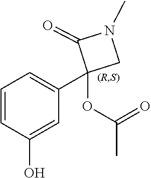 [SMILES: CC(=O)OC1(c2cccc(O)c2)CN(C)C1=O]

To a flask containing (R,S)-3-(3-((tert-butyldimethylsilyl)oxy)phenyl)-1-methyl-2-oxoazetidin-3-yl acetate (Intermediate 83, 1.2 g, 3.4 mmol) and THF (30 mL) at room temperature was added TBAF (13.7 mL, 1 M in THF, 13.7 mmol). The reaction mixture was stirred at room temperature for 3 h, then diluted with ethyl acetate (80 mL). This solution was washed with brine, dried over anhydrous Na₂SO₄, filtered, concentrated and purified by FCC (eluent: petroleum ether:ethyl acetate=1:0 to 0:1) to yield (R,S)-3-(3-hydroxyphenyl)-1-methyl-2-oxoazetidin-3-yl acetate (830 mg) as a colorless oil. ¹H NMR (400 MHz, DMSO-d₆) δ 9.61 (5, 1H), 7.28-7.20 (m, 1H), 6.97-6.87 (m, 2H), 6.83-6.74 (m, 1H), 4.00 (d, J=6.8 Hz, 1H), 3.80 (d, J=6.6 Hz, 1H), 2.87 (s, 3H), 2.16 (s, 3H).

### Intermediate 85. (R,S)-1-Methyl-2-oxo-3-(3-(((trifluoromethyl)sulfonyl)oxy)phenyl)azetidin-3-yl acetate

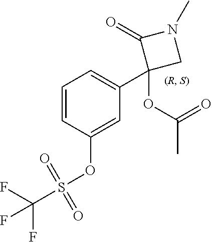 [SMILES: CC(=O)OC1(c2cccc(OS(=O)(=O)C(F)(F)F)c2)CN(C)C1=O]

To a flask containing (R,S)-3-(3-hydroxyphenyl)-1-methyl-2-oxoazetidin-3-yl acetate (Intermediate 84, 730 mg, 3.1 mmol), Et₃N (1.3 mL, 9.3 mmol), and dichloromethane (8 mL) was added trifluoromethanesulfonic anhydride (0.78 mL, 4.64 mmol). The mixture was stirred at room temperature for 12 h. The reaction mixture was combined with another batch of material, concentrated to dryness under reduced pressure, and purified by FCC (eluent: petroleum ether:ethyl acetate=1:0 to 3:1) to yield 1-methyl-2-oxo-3-(3-(((trifluoromethyl)sulfonyl)oxy)phenyl)azetidin-3-yl acetate (630 mg) as a brown oil. LCMS(ESI): mass calcd. for C₁₃H₁₂F₃NO₆S, 367.03; m/z found, 368.0[M+H]⁺. ¹H NMR (400 MHz, DMSO-d₆) δ 7.67-7.62 (m, 2H), 7.59-7.56 (m, 1H), 7.56-7.51 (m, 1H), 4.14 (d, J=7.0 Hz, 1H), 3.82 (d, J=7.0 Hz, 1H), 2.86 (s, 3H), 2.19-2.12 (m, 3H).

### Intermediate 86. (R,S)-1-Methyl-2-oxo-3-(3-(4,4,5,5-tetramethyl-1,3,2-dioxaborolan-2-yl)phenyl)azetidin-3-yl acetate

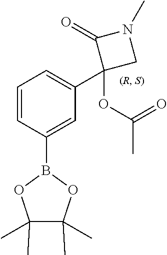 [SMILES: CC(=O)OC1(c2cccc(B3OC(C)(C)C(C)(C)O3)c2)CN(C)C1=O]

To a microwave tube containing (R,S)-1-methyl-2-oxo-3-(3-(((trifluoromethyl)sulfonyl)oxy)phenyl)azetidin-3-yl acetate (Intermediate 85, 550 mg, 0.15 mmol), 4,4,4′,4′,5,5,5′,5′-octamethyl-2,2′-bi(1,3,2-dioxaborolane (572 mg, 2.25 mmol), KOAc (441 mg, 4.49 mmol) was added 1,4-dioxane (8 mL). The mixture was sparged with Ar for 5 minutes and then X-Phos-Pd-G₂ (chloro(2-dicyclohexylphosphino-2′,4′,6′-triisopropyl-1,1′-biphenyl)[2-(2′-amino-1,1′-biphenyl)]palladium(II), CAS #1310584-14-5, 121 mg, 0.154 mmol) and X-Phos (2-dicyclohexylphosphino-2′,4′,6′-triisopropylbiphenyl, CAS #564483-18-7, 71.4 mg, 0.150 mmol) was added. The mixture was sparged with Ar for another 5 minutes and then heated at 90° C. in a microwave for 1 h. The product, 1-methyl-2-oxo-3-(3-(4,4, 5,5-tetramethyl-1,3,2-dioxaborolan-2-yl)phenyl)azetidin-3-yl acetate, was cooled to room temperature and used without further purification in next step. LCMS(ESI): mass calcd. for C₁₈H₂₄BNO₅, 345.17; m/z found, 286.2 [M-60+H]⁺.

### Intermediate 87. (R,S)-1-Methyl-2-oxo-3-(3-(2-(5-tosyl-5H-pyrrolo[2,3-b]pyrazin-7-yl)thiazol-4-yl)phenyl)azetidin-3-yl acetate

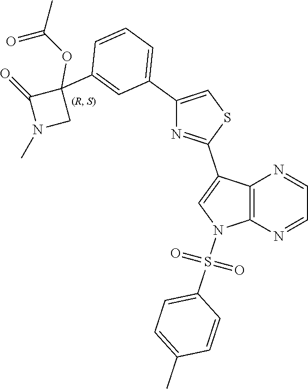 [SMILES: CC(=O)OC1(c2cccc(-c3csc(-c4cn(S(=O)(=O)c5ccc(C)cc5)c5nccnc45)n3)c2)CN(C)C1=O]

To a microwave tube containing 4-bromo-2-(5-tosyl-5H-pyrrolo[2,3-b]pyrazin-7-yl)thiazole (Intermediate 10, 260 mg, 0.60 mmol), 1-methyl-2-oxo-3-(3-(4,4,5,5-tetramethyl-1,3,2-dioxaborolan-2-yl)phenyl)azetidin-3-yl acetate (Intermediate 86, 190 mg, 0.55 mmol) and K₃PO₄ (216 mg, 1.02 mmol) was added 1,4-dioxane (12 mL), and H₂O (3 mL). The mixture was sparged with Ar for 5 minutes and then Pd(dtbpf)Cl₂ (59 mg, 0.091 mmol) was added. The mixture was sparged with Ar for another 5 minutes and then subjected to microwave irradiation at 90° C. for 1 h. The reaction mixture was cooled to room temperature, filtered through a pad of diatomaceous earth and the pad washed with ethyl acetate (20 mL). The filtrate was diluted with H₂O (30 mL) and extracted with ethyl acetate (20 mL×3). The combined organic solvent extracts were dried over anhydrous Na₂SO₄, filtered, concentrated, and purified by FCC (eluent: petroleum ether:ethyl acetate=1:0 to 0:1) to yield (R,S)-1-methyl-2-oxo-3-(3-(2-(5-tosyl-5H-pyrrolo[2,3-b]pyrazin-7-yl)thiazol-4-yl)phenyl)azetidin-3-yl acetate (250 mg, 70%) as a yellow solid. LCMS(ESI): mass calcd. for C₂₈H₂₃N₅O₅S₂, 573.11; m/z found, 574.1 [M+H]⁺.

### Intermediate 88. (S)-1-methyl-2-oxo-3-(3-(2-(5-tosyl-5H-pyrrolo[2,3-b]pyrazin-7-yl)thiazol-4-yl)phenyl)azetidin-3-yl acetate

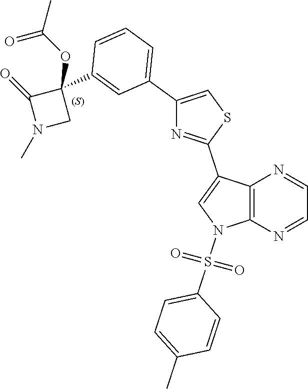

The enantiomers of methyl (R,S)-2-oxo-3-(3-(2-(5-tosyl-5H-pyrrolo[2,3-b]pyrazin-7-yl)thiazol-4-yl)phenyl)azetidin-3-yl acetate (Intermediate 87, 240 mg, 0.42 mmol) were separated by SFC using an SFC column, such as a DAICEL CHIRALCEL OD 250 mm×30 mm×10 μm column (isocratic elution: EtOH (containing 0.1% of 25% aqueous NH₃): supercritical CO₂, 45%: 55%). (S)-1-methyl-2-oxo-3-(3-(2-(5-tosyl-5H-pyrrolo[2,3-b]pyrazin-7-yl)thiazol-4-yl)phenyl)azetidin-3-yl acetate was the first eluting enantiomer and (R)-1-methyl-2-oxo-3-(3-(2-(5-tosyl-5H-pyrrolo[2,3-b]pyrazin-7-yl)thiazol-4-yl)phenyl)azetidin-3-yl acetate was the second eluting enantiomer (Intermediate 89). The pure fractions of (S)-1-methyl-2-oxo-3-(3-(2-(5-tosyl-5H-pyrrolo[2,3-b]pyrazin-7-yl)thiazol-4-yl)phenyl)azetidin-3-yl acetate were collected and concentrated under vacuum. The residue was resuspended in water (10 mL) and the mixture was frozen by insertion into a −78° C. bath. The frozen mixture was then lyophilized to yield (S)-1-methyl-2-oxo-3-(3-(2-(5-tosyl-5H-pyrrolo[2,3-b]pyrazin-7-yl)thiazol-4-yl)phenyl)azetidin-3-yl acetate (100 mg, 39%) as a white solid. SFC Rₜ=4.113 min, LCMS(ESI): mass calcd. for C₂₈H₂₃N₅O₅S₂. 573.11; m/z found, 574.2 [M+H]⁺.

### Intermediate 89. (R)-1-Methyl-2-oxo-3-(3-(2-(5-tosyl-5H-pyrrolo[2,3-b]pyrazin-7-yl)thiazol-4-yl)phenyl)azetidin-3-yl acetate

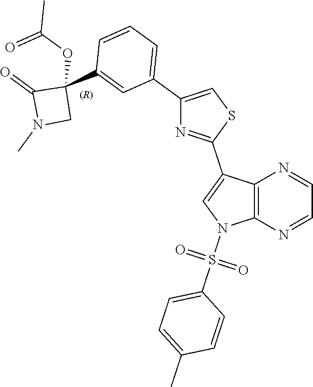

The chiral separation described in Intermediate 88 yielded (R)-1-methyl-2-oxo-3-(3-(2-(5-tosyl-5H-pyrrolo[2,3-b]pyrazin-7-yl)thiazol-4-yl)phenyl)azetidin-3-yl acetate, (SFC Rₜ=6.464 min, 110 mg, 46%) as a white solid. LCMS(ESI): mass calcd. for C₂₈H₂₃N₅O₅S₂. 573.11; m/z found, 574.2 [M+H]⁺.

### Intermediate 90: (R,S)-3-Hydroxy-1-methyl-3-(3-(2-(1-(phenylsulfonyl)-1H-pyrrolo[2,3-b]pyridin-3-yl)thiazol-4-yl)phenyl)pyrrolidin-2-one

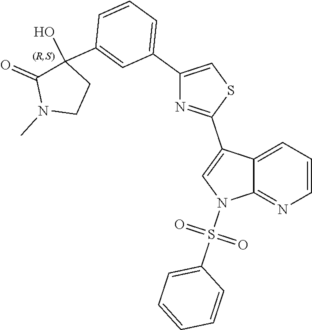

To a microwave vial containing a 2M aqueous K₂CO₃ solution (1 mL) and 1,4-dioxane (15 mL) which was degassed together with Ar for 20 min prior to use. Then 4-bromo-2-(1-(phenylsulfonyl)-1H-pyrrolo[2,3-b]pyridin-3-yl)thiazole (Intermediate 2, 201 mg, 0.480 mmol), (R,S)-3-hydroxy-1-methyl-3-(3-(4,4,5,5-tetramethyl-1,3,2-dioxaborolan-2-yl)phenyl)pyrrolidin-2-one (Intermediate 4, 205 mg, 0.650 mmol), and Pd(PPh₃)₄ (55 mg, 0.048 mmol) was added and the vial was evacuated/purged with nitrogen 3 times and placed in an aluminum heating mantle that had been heated at 90° C. After 2 h, the mixture was filtered through a pad of diatomaceous earth and the pad was rinsed with EtOAc. The filtrate was concentrated. The product was purified by FCC (using 100% DCM increasing to 100% EtOAc) to yield (R,S)-3-hydroxy-1-methyl-3-(3-(2-(1-(phenylsulfonyl)-1H-pyrrolo[2,3-b]pyridin-3-yl)thiazol-4-yl)phenyl)pyrrolidin-2-one (303 mg) as a light pink amorphous solid. MS (ESI): mass calcd. for C₂₇H₂₂N₄O₄S₂, 530.63; m/z found, 531.05 [M+H]⁺. ¹H NMR (500 MHz, CDCl₃) δ 8.70 (dd, J=7.9, 1.6 Hz, 1H), 8.55-8.49 (m, 1H), 8.32 (s, 1H), 8.29-8.25 (m, 2H), 8.07-8.03 (m, 1H), 7.96-7.91 (m, 1H), 7.63-7.58 (m, 1H), 7.55-7.48 (m, 3H), 7.45-7.48 (m, 1H), 7.39-7.31 (m, 2H), 3.53-3.35 (m, 2H), 3.24 (s, 1H), 3.05 (s, 3H), 2.60-2.43 (m, 2H).

### Intermediate 91. (R)-3-Hydroxy-3-(3-(2-(5-methoxy-1-tosyl-1H-pyrrolo[2,3-b]pyridin-3-yl)thiazol-4-yl)phenyl)-1-methylpyrrolidin-2-one

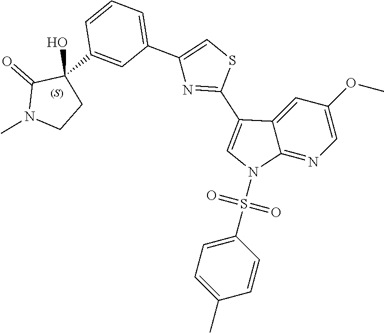

Step A. 4-Bromo-2-(5-methoxy-1-tosyl-1H-pyrrolo[2,3-b]pyridin-3-yl)thiazole. A vial containing 1,4-dioxane (20 mL) and 2M aqueous K₂CO₃ (2 mL, 4.2 mmol) was degassed together for 15 min with nitrogen. 5-methoxy-3-(4,4,5,5-tetramethyl-1,3,2-dioxaborolan-2-yl)-1-tosyl-1H-pyrrolo[2,3-b]pyridine (450 mg, 1.05 mmol), 2,4 dibromothiazole (255 mg, 1.05 mmol), and Pd(PPh₃)₄ (121 mg, 0.11 mmol) was then added. The vial was sealed and evacuated/purged with nitrogen 3 times and placed in an aluminum heating mantle at 110° C. After 3.25 h, the reaction mixture was filtered through a pad of diatomaceous earth while still warm and the pad was rinsed further with EtOAc and THF. The filtrate was concentrated to yield a dark brownish gum. The gum diluted with CH₃CN and vortexed, which resulted in grey solid which was collected by suction filtration. The solid was rinsed with CH₃CN and then Et₂O and dried under reduced pressure to yield 4-bromo-2-(5-methoxy-1-tosyl-1H-pyrrolo[2,3-b]pyridin-3-yl)thiazole (230 mg). MS (ESI): mass calcd. for C₁₈H₁₄BrN₃O₃S₂, 464.36; m/z found, 466.20 [M+H]⁺. ¹H NMR (400 MHz, Chloroform-d) δ 8.23 (d, J=1.8 Hz, 2H), 8.12-8.05 (m, 2H), 8.01 (d, J=2.9 Hz, 1H), 7.31-7.27 (m, 2H), 7.19 (s, 1H), 3.92 (s, 3H), 3.70 (s, 1H), 2.38 (s, 3H).

Step B. (R)-3-Hydroxy-3-(3-(2-(5-methoxy-1-tosyl-1H-pyrrolo[2,3-b]pyridin-3-yl)thiazol-4-yl)phenyl)-1-methylpyrrolidin-2-one. A vial containing 1,4-dioxane (8 mL) and 2M aqueous K₂CO₃ (0.55 mL, 1.4 mmol) was degassed together with nitrogen for 20 min. Then, 4-bromo-2-(5-methoxy-1-tosyl-1H-pyrrolo[2,3-b]pyridin-3-yl)thiazole (150 mg, 0.32 mmol), (R)-3-hydroxy-1-methyl-3-(3-(4,4,5,5-tetramethyl-1,3,2-dioxaborolan-2-yl)phenyl)pyrrolidin-2-one (Intermediate 16, 112 mg, 0.350 mmol), and Pd(PPh₃)₄ (38 mg, 0.033 mol) was added. The vial was sealed and evacuated/purged with nitrogen 3 times and placed in an aluminum heating mantle at 100° C. After 1.5 h, the reaction mixture was filtered through a pad of diatomaceous earth and rinsed with EtOAc and THF. The filtrate was concentrated to an amber oil and purified by FCC (using 100% DCM increasing to 5% MeOH-DCM) to yield (R)-3-hydroxy-3-(3-(2-(5-methoxy-1-tosyl-1H-pyrrolo[2,3-b]pyridin-3-yl)thiazol-4-yl)phenyl)-1-methylpyrrolidin-2-one as an off-white amorphous solid, which was used without further purification. MS (ESI): mass calcd. for C₂₉H₂₆N₄O₅S₂, 574.68; m/z found, 576.20 [M+H]⁺. ¹H NMR (400 MHz, Chloroform-d) δ 8.20 (d, J=0.9 Hz, 1H), 8.13-8.05 (m, 1H), 7.46-7.23 (m, 3H), 3.93 (s, 1H), 3.51-3.31 (m, 1H), 3.01 (d, J=8.7 Hz, 1H), 2.57-2.40 (m, 1H), 2.37 (s, 1H), 1.33 (s, 1H).

### Intermediate 92. (R)-3-Hydroxy-3-(3-(5-(hydroxymethyl)-2-(1-tosyl-1H-pyrrolo[2,3-b]pyridin-3-yl)thiazol-4-yl)phenyl)-1-methylpyrrolidin-2-one

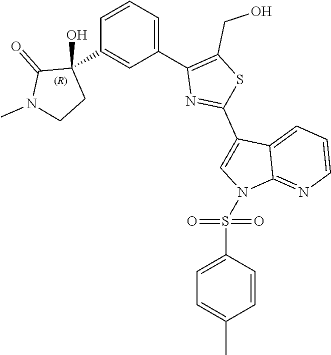

Step A. (4-Bromo-2-(1-tosyl-1H-pyrrolo[2,3-b]pyridin-3-yl)thiazol-5-yl)methanol. A solution of 2M aqueous K₂CO₃ (0.74 mL 0.1.47 mmol) and 1,4-dioxane (10 mL) was degassed with N₂ for 15 min prior to use. Then (2,4-dibromothiazol-5-yl)methanol (100 mg, 0.37 mmol) and 3-(4,4,5,5-tetramethyl-1,3,2-dioxaborolan-2-yl)-1-tosyl-1H-pyrrolo[2,3-b]pyridine (146 mg, 0.370 mmol) was added followed by PdCl₂(dppf) (27 mg, 0.037 mmol). The vial was sealed and evacuated/purged with nitrogen 3 times and then placed in an aluminum heating mantle at 90° C. After 2 h, the reaction mixture was filtered through a pad of diatomaceous earth and the pad was rinsed with EtOAc. The filtrate was concentrated to yield a brownish gum which was re-dissolved in CHCl₃ and diatomaceous earth (3 g) was added and the material was concentrated to dryness and purified by FCC (using 100% Hexanes/0% EtOAc to 0% Hexanes/100% EtOAc) to yield (4-bromo-2-(1-tosyl-1H-pyrrolo[2,3-b]pyridin-3-yl)thiazol-5-yl)methanol (121 mg) as a pale yellow solid. MS (ESI): mass calcd. for C₁₈H₁₄BrN₃O₃S₂, 464.36; m/z found, 465.90 [M+H]⁺. ¹H NMR (400 MHz, Chloroform-d) δ 8.56-8.43 (m, 2H), 8.28 (s, 1H), 8.17-8.10 (m, 3H), 7.34-7.27 (m, 4H), 4.88 (d, J=6.1 Hz, 2H), 2.38 (s, 3H).

Step B. (R)-3-Hydroxy-3-(3-(5-(hydroxymethyl)-2-(1-tosyl-1H-pyrrolo[2,3-b]pyridin-3-yl)thiazol-4-yl)phenyl)-1-methylpyrrolidin-2-one. Two side-by-side reactions were carried out in microwave vials each containing 1,4-dioxane (8 mL) and 2M aqueous K₂CO₃ (0.5 mL) and these solutions were degassed with nitrogen for 20 min. Then, (4-bromo-2-(1-tosyl-1H-pyrrolo[2,3-b]pyridin-3-yl)thiazol-5-yl)methanol (90 m, 0.19 mmol), (R)-3-hydroxy-1-methyl-3-(3-(4,4,5,5-tetramethyl-1,3,2-dioxaborolan-2-yl)phenyl)pyrrolidin-2-one (Intermediate 16, 82 mg, 0.26 mmol), and PdCl₂(PPh₃)₂ (15 mg, 0.022 mmol) were added. The vials were sealed and evacuated/purged with nitrogen 3 times and placed in an aluminum heating mantle at 100° C. After 1.75 h, the contents from each reaction were filtered through a pad of diatomaceous earth while still warm and the diatomaceous earth pad was rinsed with EtOAc. The filtrate was concentrated to a brownish oil which was re-dissolved with CHCl₃, diatomaceous earth (3.5 g) was then added and the mixture was concentrated. The resultant residue was purified by FCC (using 0% MeOH/100% DCM to 10% MeOH/90% DCM) to yield the product as a viscous amber gum. Subsequent trituration of this gum with Et₂O and drying under vacuum yielded (R)-3-hydroxy-3-(3-(5-(hydroxymethyl)-2-(1-tosyl-1H-pyrrolo[2,3-b]pyridin-3-yl)thiazol-4-yl)phenyl)-1-methylpyrrolidin-2-one (202 mg) as a light amber amorphous solid. MS (ESI): mass calcd. for C₂₉H₂₆N₄O₅S₂, 574.68; m/z found, 575.10 [M+H]⁺. ¹H NMR (500 MHz, Chloroform-d) δ 8.56 (dd, J=8.0, 1.6 Hz, 1H), 8.46 (dd, J=14.8, 1.6 Hz, 1H), 8.26 (s, 1H), 8.17-8.09 (m, 2H), 7.76-7.70 (m, 1H), 7.64-7.67 (m, 1H), 7.47-7.35 (m, 2H), 7.32-7.21 (m, 3H), 4.82 (d, J=5.2 Hz, 2H), 3.84 (br s, 1H), 3.52-3.43 (m, 2H), 3.37-3.40 (m, 1H), 3.24 (br s, 1H), 3.01 (s, 3H), 2.57-2.40 (m, 2H), 2.37 (s, 3H).

### Intermediate 93. Methyl (R)-3-(4-(3-(3-hydroxy-1-methyl-2-oxopyrrolidin-3-yl)phenyl)thiazol-2-yl)-1-tosyl-1H-pyrrolo[2,3-b]pyridine-5-carboxylate

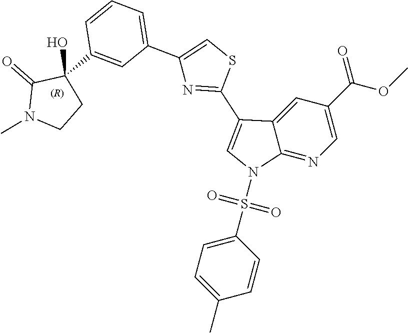

Step A. Methyl 3-bromo-1-tosyl-1H-pyrrolo[2,3-b]pyridine-5-carboxylate. To a 1,000 mL 3-necked flask fitted with an addition funnel, was added NaH (60% in mineral oil, 3.03 g, 75.7 mmol) and THF (300 mL) and the grey suspension was cooled to 0° C.; in an ice-water bath. Then, methyl 3-bromo-1H-pyrrolo[2,3-b]pyridine-5-carboxylate (14.7 g in 150 mL THF, 14.7 mmol) was added over 5 min via an addition funnel. After 20 min at 0° C., the mixture was warmed to room temperature for 60 min, then re-cooled to 0° C. and p-toluenesulfonyl chloride (16.1 g, 84.65 mmol, in 100 mL THF) was added over 3 min which yielded a suspension. Once the addition was complete, the ice bath was removed, and the mixture was allowed to warm room temperature. After 1.75 h, half of the solvent was removed under reduced pressure and the reaction was quenched with aqueous saturated NH₄Cl solution. The aqueous portion was extracted with EtOAc (2×100 mL) and then with CHCl₃ (2×100 mL). The organic solvent extracts were washed with brine, dried over MgSO₄, filtered and concentrated. The resultant solid was triturated with hexanes:ether (9:1) which yielded methyl 3-bromo-1-tosyl-1H-pyrrolo[2,3-b]pyridine-5-carboxylate (21.7 g). ¹H NMR (500 MHz, CDCl₃) δ 9.09 (d, J=1.7 Hz, 1H), 8.45 (d, J=1.7 Hz, 1H), 8.09 (d, J=8.3 Hz, 2H), 7.86 (s, 1H), 7.30 (d, J=8.2 Hz, 2H), 3.97 (s, 3H), 2.38 (s, 3H).

Step B. Methyl 3-(4,4,5,5-tetramethyl-1,3,2-dioxaborolan-2-yl)-1-tosyl-1H-pyrrolo[2,3-b]pyridine-5-carboxylate. To a flask was added methyl 3-(4-bromothiazol-2-yl)-1-tosyl-1H-pyrrolo[2,3-b]pyridine-5-carboxylic acid (5,008 mg, 12.240 mmol), B₂(Pin)₂ (6,290 mg, 24.77 mmol), KOAc (3,680 mg, 37.5 mmol), XPhos gen2 pd (chloro(2-dicyclohexylphosphino-2′,4′,6′-triisopropyl-1,1′-biphenyl)[2-(2′-amino-1,1′-biphenyl)]palladium(II), CAS #1310584-14-5, 65 mg, 1.23 mmol) and 1,4-dioxane (degassed with N₂ for 20 min prior to use). The flask was fitted with a reflux condenser and evacuated/purged with Ar and then placed in an aluminum heating mantle at 110° C. with an Ar balloon on top of the condenser. After 2 h, the reaction mixture was cooled to room temperature and then filtered through a pad of diatomaceous earth which was rinsed further with EtOAc. The filtrate was concentrated initially to a brown gum, which upon standing became a solid. The solid was then triturated with hexanes and the resulting greyish solid was collected by suction filtration which yielded methyl 3-(4,4,5,5 tetramethyl-1,3,2-dioxaborolan-2-yl)-1-tosyl-1H-pyrrolo[2,3-b]pyridine-5-carboxylic acid (2.95 g) as a light tan solid. LCMS (ESI): mass calcd. for C₂₂H₂₅BN₂O₆S, 456.33; m/z found, 456.10 [M+H]⁺. ¹H NMR (500 MHz, Chloroform-d) δ 9.05 (d, J=2.0 Hz, 1H), 8.77 (d, J=2.1 Hz, 1H), 8.20 (s, 1H), 8.14-8.05 (m, 2H), 7.33-7.16 (m, 4H), 3.96 (s, 3H), 3.70 (s, 1H), 2.37 (s, 3H), 1.36 (s, 12H), 1.27 (d, J=4.7 Hz, 3H).

Step C. Methyl 3-(4-bromothiazol-2-yl)-1-tosyl-1H-pyrrolo[2,3-b]pyridine-5-carboxylate. A solution of 1,4-dioxane (20 mL) and 2M aqueous K₂CO₃ (2 mL, 4.38 mmol) was degassed for 20 min with nitrogen. Then, methyl 3-(4,4,5,5-tetramethyl-1,3,2-dioxaborolan-2-yl)-1-tosyl-1H-pyrrolo[2,3-b]pyridine-5-carboxylate (500 mg, 1.1 mmol), 2,4-dibromothiazole (266 mg, 1.10 mmol), and Pd(PPh₃)₄ (126 mg, 0.110 mmol) were added. The vial was sealed and evacuated/purged with nitrogen 3 times and placed in an aluminum heating mantle at 110° C. After 2 h, the reaction mixture was filtered through a pad of diatomaceous earth (while still warm) and the pad was rinsed further with EtOAc and THF. The filtrate was concentrated to a solid which was triturated with EtOAc. The resulting solid was collected by suction filtration and rinsed further with EtOAc and then Et₂O, and dried to yield methyl 3-(4-bromothiazol-2-yl)-1-tosyl-1H-pyrrolo[2,3-b]pyridine-5-carboxylate (300 mg) as an off-white solid. LCMS (ESI): mass calcd. for C₁₉H₁₄BrN₃O₄S₂, 492.37; m/z found, 492.0 [M+H]⁺. ¹H NMR (500 MHz, Chloroform-d) δ 9.12 (dd, J=J=22.9, 2.1 Hz, 2H), 8.37 (s, 1H), 8.19-8.10 (m, 2H), 7.37-7.19 (m, 3H), 3.98 (s, 3H), 2.39 (s, 3H).

Step D. Methyl (R)-3-(4-(3-(3-hydroxy-1-methyl-2-oxopyrrolidin-3-yl)phenyl)thiazol-2-yl)-1-tosyl-1H-pyrrolo[2,3-b]pyridine-5-carboxylate. To a large microwave vial containing 1,4-dioxane (20 mL) and 2M aqueous K₂CO₃ (1.2 mL, 2.4 mmol) was degassed together with nitrogen for 20 min. Then, methyl 3-(4-bromothiazol-2-yl)-1-tosyl-1H-pyrrolo[2,3-b]pyridine-5-carboxylate (250 mg, 0.51 mmol), (R)-3 hydroxy-1-methyl-3-(3-(4,4,5,5-tetramethyl-1,3,2-dioxaborolan-2-yl)phenyl)pyrrolidin-2-one (Intermediate 16, 177 mg, 0.56 mol), and Pd(PPh₃)₄ (60 mg, 0.052 mmol) were added. The vial was sealed and evacuated/purged with nitrogen 3 times and placed in an aluminum heating mantle at 110° C. After 2 h, the reaction mixture was filtered through a pad of diatomaceous earth and the pad was rinsed further with EtOAc and THF. The filtrate was concentrated and purified by FCC (using 0% 2M NH₃ in MeOH/100% DCM to 5% 2M NH₃ in MeOH/95% DCM) to yield methyl (R)-3-(4-(3-(3-hydroxy-1-methyl-2-oxopyrrolidin-3-yl)phenyl)thiazol-2-yl)-1-tosyl-1H-pyrrolo[2,3-b]pyridine-5-carboxylate (310 mg) as a light amber amorphous solid. This material was used in the next step without further purification. ¹H NMR (500 MHz, CDCl₃) δ 9.36-9.27 (m, 1H), 9.14 (t, J=2.2 Hz, 1H), 8.41-8.29 (m, 1H), 8.18-8.13 (m, 2H), 8.10-8.12 (m, 1H), 7.95-7.87 (m, 1H), 7.73-7.58 (m, 2H), 7.57-7.39 (m, 5H), 7.37-7.28 (m, 4H), 3.99 (d, J=1.1 Hz, 3H), 3.56-3.29 (m, 4H), 3.11-2.93 (m, 4H), 2.63-2.46 (m, 2H), 2.39 (s, 3H).

### Intermediate 94. (R,S)-3-Hydroxy-1-methyl-3-(3-(2-(7-tosyl-7H-pyrrolo[2,3-d]pyrimidin-5-yl)thiazol-4-yl)phenyl)pyrrolidin-2-one

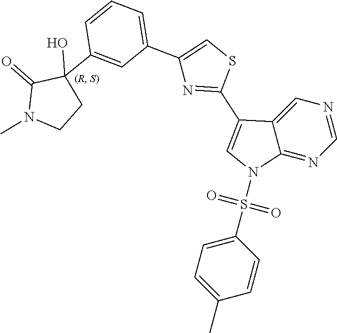

Step A. 4-bromo-2-(7-tosyl-7H-pyrrolo[2,3-d]pyrimidin-5-yl)thiazole. A solution of dioxane (50 mL) and 2M aqueous K₂CO₃ (6.5 mL, 12.5 mmol) was degassed with Ar for 25 min, then 5-(4,4,5,5-tetramethyl-1,3,2-dioxaborolan-2-yl)-7-tosyl-7H-pyrrolo[2,3-d]pyrimidine (1007 mg, 2.52 mmol), 2,4-dibromothiazole (727 mg, 2.99 mmol), and Pd(PPh₃)₄ (288 mg, 0.25 mmol) was added. The flask was fitted with a reflux condenser, sealed and evacuated/purged with nitrogen 3 times and placed in an aluminum heating mantle at 90° C. After 2 h, the reaction mixture was filtered through a pad of diatomaceous earth while still warm and the pad was rinsed with EtOAc and THF. The filtrate was concentrated to provide a solid which was triturated with CH₃CN and the resulting solid was collected via suction filtration to yield 4-bromo-2-(7-tosyl-7H-pyrrolo[2,3-d]pyrimidin-5-yl)thiazole (740 mg) as light salmon colored solid. LCMS (ESI): mass calcd. for C₁₆H₁₁BrN₄O₂S₂, 435.32; m/z found, 437.95 [M+H]⁺. ¹H NMR (500 MHz, CDCl₃) δ 9.59 (s, 1H), 9.10 (s, 1H), 8.28 (s, 1H), 8.18-8.12 (m, 2H), 7.35 (d, J=8.1 Hz, 2H), 7.25 (s, 1H), 2.41 (s, 3H).

Step B. 3-Hydroxy-1-methyl-3-(3-(2-(7-tosyl-7H-pyrrolo[2,3-d]pyrimidin-5-yl)thiazol-4-yl)phenyl)pyrrolidin-2-one. To a microwave vial containing 4-bromo-2-(7-tosyl-7H-pyrrolo[2,3-d]pyrimidin-5-yl)thiazole (Intermediate 7, 175 mg, 0.4 mmol), (R,S)-3-hydroxy-1-methyl-3-(3-(4,4,5,5-tetramethyl-1,3,2-dioxaborolan-2-yl)phenyl)pyrrolidin-2-one (Intermediate 4, 171 mg, 0.54 mmol), and Pd(PPh₃)₄ (48 mg, 0.042 mmol) was added a solution of 1,4-dioxane (12 mL) and 2M aqueous K₂CO₃. The K₂CO₃/1,4-dioxane solution was degassed with Ar for 15 min prior to use. The vial was evacuated/purged with nitrogen 3 times and placed in an aluminum heating mantle at 90° C. After 2 h, the mixture was filtered through a pad of diatomaceous earth and the pad was rinsed with EtOAc. The filtrate was concentrated and purified by FCC (using 0% MeOH/100° DCM to 5% MeOH/95% DCM) to yield (R,S)-3-hydroxy-1-methyl-3-(3-(2-(7-tosyl-7H-pyrrolo[2,3-d]pyrimidin-5-yl)thiazol-4-yl)phenyl)pyrrolidin-2-one (191 mg) as a light amber amorphous solid. LCMS (ESI): mass calcd. for C₂₇H₂₃N₅O₄S₂, 545.64; m/z found, 546.05 [M+H]⁺. ¹H NMR (500 MHz, CDCl₃) δ 9.74 (s, 1H), 9.10 (s, 1H), 8.26 (s, 1H), 8.21-8.13 (m, 2H), 8.03 (t, J=1.6 Hz, 1H), 7.92 (d, J=7.7 Hz, 1H), 7.51 (s, 1H), 7.49-7.41 (m, 1H), 7.40-7.31 (m, 3H), 3.53-3.39 (m, 2H), 3.35 (br s, 1H), 3.06 (s, 3H), 2.60-2.43 (m, 2H), 2.41 (s, 3H).

### Intermediate 95. (S)-7-(3-(4-(5-Tosyl-5H-pyrrolo[2,3-b]pyrazin-7-yl)thiazol-2-yl)phenyl)-6,7-dihydro-5H-pyrrolo[1,2-a]imidazol-7-ol

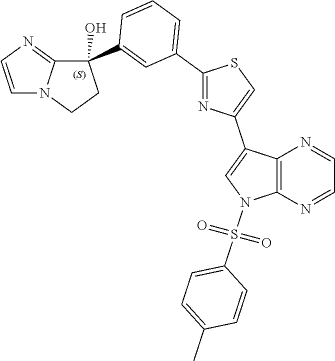

Step A. (S)-7-(3-(4-Bromothiazol-2-yl)phenyl)-6,7-dihydro-5H-pyrrolo[1,2-a]imidazol-7-ol. To a tube containing (S)-7-(3-(4,4,5,5-tetramethyl-1,3,2-dioxaborolan-2-yl)phenyl)-6,7-dihydro-5H-pyrrolo[1,2-a]imidazol-7-ol (Intermediate 39, 220 mg, 0.67 mmol), 2,4-dibromothiazole (246 mg, 1.01 mmol), and Cs₂CO₃ (549 mg, 1.69 mmol), was added 1,4-dioxane (10 mL), and H₂O (2 mL). The mixture was sparged with Ar for 5 minutes and then Pd(dppf)Cl₂ (49 mg, 0.07 mmol) was added. The mixture was sparged with Ar for another 5 minutes and then subjected to microwave irradiation at 80° C. for 1 h. The reaction mixture was then cooled to room temperature and concentrated to dryness. The product was purified by FCC (eluent: petroleum ether:ethyl acetate=5:1 to 0:1) to yield (S)-7-(3-(4-bromothiazol-2-yl)phenyl)-6,7-dihydro-5H-pyrrolo[1,2-a]imidazol-7-ol (190 mg, 47%) as a brown solid. LCMS (ESI): mass calcd. for C₁₅H₁₂BrN₃OS, 360.99; m/z found, 362.0 [M+H]⁺. ¹H NMR (400 MHz, DMSO-d₆) δ 8.12-8.08 (m, 1H), 7.91 (s, 1H), 7.86-7.82 (m, 1H), 7.53-7.48 (m, 2H), 7.22-7.16 (m, 1H), 7.04-7.01 (m, 1H), 6.28 (s, 1H), 4.15-4.01 (m, 2H), 2.85-2.76 (m, 2H).

Step B. (S)-7-(3-(4-(5-Tosyl-5H-pyrrolo[2,3-b]pyrazin-7-yl)thiazol-2-yl)phenyl)-6,7-dihydro-5H-pyrrolo[1,2-a]imidazol-7-ol. To a microwave tube containing (S)-7-(3-(4-bromothiazol-2-yl)phenyl)-6,7-dihydro-5H-pyrrolo[1,2-a]imidazol-7-ol (180 mg, 0.497 mmol), 7-(4,4,5,5-tetramethyl-1,3,2-dioxaborolan-2-yl)-5-tosyl-5H-pyrrolo[2,3-b]pyrazine (Intermediate 9, 298 mg, 0.746 mmol), and K₃PO₄ (316 mg, 1.49 mmol) was added 1,4-dioxane (10 mL), and H₂O (2 mL). The mixture was sparged with Ar for 5 minutes and then Pd(dtbpf)Cl₂ (32 mg, 0.05 mmol) was added. The mixture was sparged with Ar for another 5 minutes and subjected to microwave irradiation at 85° C. for 1 h. The reaction mixture was cooled to room temperature and concentrated to dryness. The product was purified by FCC (eluent: petroleum ether ethyl acetate=10:1 to 0:1) to yield (S)-7-(3-(4-(5-tosyl-5H-pyrrolo[2,3-b]pyrazin-7-yl)thiazol-2-yl)phenyl)-6,7-dihydro-5H-pyrrolo[1,2-a]imidazol-7-ol (90 mg, 26%) as a brown solid. LCMS (ESI): mass calcd. for C₂₈H₂₂N₆O₃S₂, 554.12; m/z found, 555.1 [M+H]⁺.

### Intermediate 96. (R,S)-4-(3-(2-(5-(Phenylsulfonyl)-5H-pyrrolo[2,3-b]pyrazin-7-yl)thiazol-4-yl)phenyl)-5,6-dihydro-4H-cyclopenta[d]thiazol-4-ol

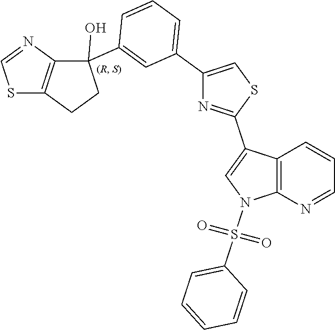

4-Bromo-2-(1-(phenylsulfonyl)-1H-pyrrolo[2,3-b]pyridin-3-yl)thiazole (Intermediate 2, 2.0 g, 4.8 mmol), (R,S)-4-(3-(4,4,5,5-tetramethyl-1,3,2-dioxaborolan-2-yl)phenyl)-5,6-dihydro-4H-cyclopenta[d]thiazol-4-ol (Intermediate 63, 3.0 g, 8.7 mmol), and K₃PO₄ (3.0 g, 14 mmol) were added to 1,4-dioxane (40 mL) and H₂O (10 mL). The mixture was sparged with Ar for 5 minutes and then treated with Pd(dtbpf)Cl₂ (310 mg, 0.476 mmol). The mixture was sparged with Ar for another 5 minutes and then heated at 100° C. for 16 h. The reaction mixture was cooled to room temperature, concentrated to dryness, and purified by FCC (eluent: petroleum ether:ethyl acetate=10:1 to 0:1, then ethyl acetate:MeOH=5:1) to yield (R,S)-4-(3-(2-(5-(phenylsulfonyl)-5H-pyrrolo[2,3-b]pyrazin-7-yl)thiazol-4-yl)phenyl)-5,6-dihydro-4H-cyclopenta[d]thiazol-4-ol (1.19 g, 29% yield) as a red oil. LCMS (ESI): mass calcd. for C₂₈H₂₀N₄O₃S₃, 556.07; m/z found, 557.0 [M+1]⁺.

### Intermediate 97. (R)-3-Hydroxy-1-methyl-3-(3-(4-(trimethylstannyl)thiazol-2-yl)phenyl)pyrrolidin-2-one

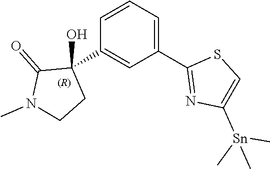

To a flask containing 1,1,1,2,2,2-hexamethyldistannane (1.4 g, 4.3 mmol), (R)-3-(3-(4-bromothiazol-2-yl)phenyl)-3-hydroxy-1-methylpyrrolidin-2-one (Intermediate 7, 1.0 g, 2.8 mmol), and toluene (10 mL) was added tetrakis(triphenylphosphine)palladium(0) (327 mg, 0.283 mmol). The resultant mixture was heated at 110° C. for 16 h. After this time, the mixture was cooled to room temperature, treated with saturated KF (100 mL), stirred for 2 h, and then extracted with ethyl acetate (50 mL×3). The combined organic solvent extracts were washed with brine (50 mL), dried over anhydrous Na₂SO₄, filtered, concentrated and partially purified by FCC (eluent: petroleum ether:ethyl acetate=1:0 to 0:1). This material was further purified by preparative HPLC using an HPLC column, such as a Boston Prime C18 150×40 mm×5 μm column (eluent: 40% to 70%, CH₃CN and H₂O (with 0.05% NH₄OH)). The product was suspended in water (10 mL), and the mixture was frozen by insertion into a −78° C. bath. The frozen mixture was then lyophilized to provide (R)-3-hydroxy-1-methyl-3-(3-(4-(trimethylstannyl)thiazol-2-yl)phenyl)pyrrolidin-2-one (247.1 mg, 20%) as a white solid. LCMS(ESI): mass calcd. for C₁₇H₂₂N₂O₂SSn, 438.04; m/z found, 438.9 [M+H]⁺. ¹H NMR (400 MHz, CDCl₃) δ 8.04 (s, 1H), 7.94-7.86 (m, 1H), 7.45-7.33 (m, 3H), 3.50-3.37 (m, 2H), 3.25 (s, 1H), 3.05 (s, 3H), 2.57-2.43 (m, 2H), 0.52-0.28 (m, 9H).

### Intermediate 98. 3-Bromo-2-fluoro-1-tosyl-1H-pyrrolo[2,3-b]pyridine

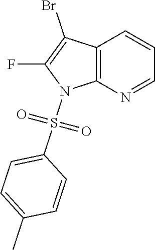 [SMILES: Cc1ccc(S(=O)(=O)n2c(F)c(Br)c3cccnc32)cc1]

Lithium diisopropylamide (2.0 mL, 2.0 M in THF, 4 mmol) was added dropwise to a solution of 3-bromo-1-tosyl-1H-pyrrolo[2,3-b]pyridine (1.0 g, 2.8 mmol) and THF (15 mL) that had been cooled to −60° C. The resultant mixture was stirred at −60° C. for 1 h, then treated with a solution of N-fluoro-N-(phenylsulfonyl)benzenesulfonamide (1.1 g, 3.5 mmol) and THF (5 mL) and stirred for 16 h with gradual warming to room temperature. After this time, the mixture was quenched with aqueous saturated NH₄Cl solution (20 mL) and extracted with ethyl acetate (20 mL×3). The combined organic solvent extracts were washed with brine (10 mL), dried over anhydrous Na₂SO₄, filtered, concentrated and purified by preparative HPLC using an HPLC column, such as a Phenomenex Gemini-NX 150 mm×30 mm×5 μm column (eluent: 50% to 80%, CH₃CN and H₂O (with 0.05% NH₄OH)) to yield the product. This material was suspended in water (10 mL) and the mixture was frozen by insertion into a −78° C. bath. The frozen mixture was then lyophilized to yield 3-bromo-2-fluoro-1-tosyl-1H-pyrrolo[2,3-b]pyridine (300 mg, 27%) as a white solid. LCMS (ESI): mass calcd. for C₁₄H₁₀BrFN₂O₂S, 367.96; m/z found, 368.9 [M+H]⁺. ¹H NMR (400 MHz, DMSO-d₆) δ 8.43 (dd, J=4.9, 1.4 Hz, 1H), 7.98 (d, J=8.3 Hz, 2H), 7.89 (dd, J=7.9, 1.6 Hz, 1H), 7.47-7.38 (m, 3H), 2.34 (s, 3H).

### Intermediate 99. (R)-3-(3-(4-(2-fluoro-1-tosyl-1H-pyrrolo[2,3-b]pyridin-3-yl)thiazol-2-yl)phenyl)-3-hydroxy-1-methylpyrrolidin-2-one

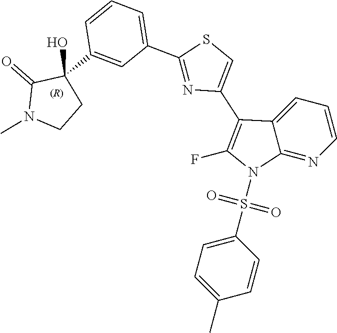

To a flask containing (R)-3-hydroxy-1-methyl-3-(3-(4-(trimethylstannyl)thiazol-2-yl)phenyl)pyrrolidin-2-one (Intermediate 97, 150 mg, 0.343 mmol), and 1,4-dioxane (3 mL) was added 3-bromo-2-fluoro-1-tosyl-1H-pyrrolo[2,3-b]pyridine (Intermediate 98, 190 mg, 0.515 mmol). The mixture was sparged with Ar for 5 minutes and then treated with Pd(t-Bu₃P)₂ (18 mg, 0.034 mmol) and CsF (26 mg, 0.17 mmol). The resultant mixture was purged with Ar for another 5 minutes and then heated at 90° C. for 16 h. After this time, the reaction mixture was cooled to room temperature, concentrated to dryness, and purified by FCC (eluent: petroleum ether:ethyl acetate=1:0 to 0:1) to yield (R)-3-(3-(4-(2-fluoro-1-tosyl-1H-pyrrolo[2,3-b]pyridin-3-yl)thiazol-2-yl)phenyl)-3-hydroxy-1-methylpyrrolidin-2-one (148 mg, 68%) as a yellow solid. LCMS (ESI): mass calcd. for C₂₈H₂₃FN₄O₄S₂, 562.11; m/z found, 563.0 [M+H]⁺.

### Intermediate 100. (2-Fluoro-1-tosyl-1H-pyrrolo[2,3-b]pyridin-3-yl)boronic acid

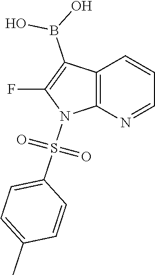 [SMILES: Cc1ccc(S(=O)(=O)n2c(F)c(B(O)O)c3cccnc32)cc1]

n-Butyllithium (1.1 mL, 2.5 M in hexane, 2.7 mmol) was added dropwise to a solution of 3-bromo-2-fluoro-1-tosyl-1H-pyrrolo[2,3-b]pyridine (Intermediate 98, 500 mg, 1.35 mmol), triisopropyl borate (0.63 mL, 2.7 mmol), and THE (8 mL) that had been cooled to −72° C. The resultant mixture was stirred at −72° C. for 30 minutes. After this time, the mixture was quenched with water (3 mL), the pH was adjusted to pH=6 with 1 M HCl, and then it was extracted with ethyl acetate (10 mL×2). The combined organic solvent extracts were dried over anhydrous Na₂SO₄, filtered, and concentrated under reduced pressure to yield 2-fluoro-1-tosyl-1H-pyrrolo[2,3-b]pyridin-3-yl)boronic acid (480 mg) as a yellow oil. LCMS (ESI): mass calcd. for C₁₄H₁₂BFN₂O₄S, 334.13; m/z found, 335.0 [M+H]⁺.

### Intermediate 101. (R)-3-(3-(2-(2-Fluoro-1-tosyl-1H-pyrrolo[2,3-b]pyridin-3-yl)thiazol-4-yl)phenyl)-3-hydroxy-1-methylpyrrolidin-2-one

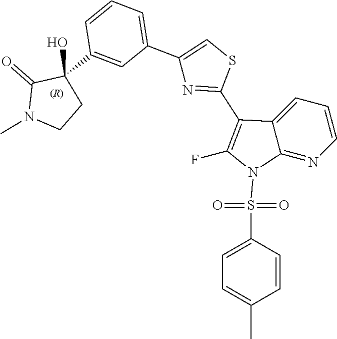

To a microwave tube containing 2-fluoro-1-tosyl-1H-pyrrolo[2,3-b]pyridin-3-yl)boronic acid (Intermediate 100, 480 mg, 0.790 mmol), (R)-3-(3-(2-bromothiazol-4-yl)phenyl)-3-hydroxy-1-methylpyrrolidin-2-one (Intermediate 187, 223 mg, 0.632 mmol), K₃PO₄ (503 mg, 2.37 mmol), and 1,4-dioxane/H₂O (6 mL, 4:1) was added [1,1-bis(di-tert-butylphosphino)ferrocene]dichloropalladium(II) (51 mg, 0.079 mmol). The resultant mixture was subjected to microwave irradiation at 80° C. for 1 h. After this time, the mixture was cooled to room temperature, and concentrated to dryness under reduced pressure to yield (R)-3-(3-(2-(2-fluoro-1-tosyl-1H-pyrrolo[2,3-b]pyridin-3-yl)thiazol-4-yl)phenyl)-3-hydroxy-1-methylpyrrolidin-2-one (510 mg) as a black solid. LCMS (ESI): mass calcd. for C₂₈H₂₃FN₄O₄S₂, 562.11; m/z found, 563.1 [M+H]⁺.

### Intermediate 102: (S) 4-(3-(4,4,5,5-Tetramethyl-1,3,2-dioxaborolan-2-yl)phenyl)-5,6-dihydro-4H-cyclopenta[d]thiazol-4-ol

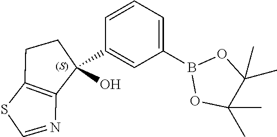

(S) 4-(3-(4,4,5,5-Tetramethyl-1,3,2-dioxaborolan-2-yl)phenyl)-5,6-dihydro-4H-cyclopenta[d]thiazol-4-ol was obtained as a white solid, by the separation of the enantiomers of (R,S)-4-(3-(4,4,5,5-tetramethyl-1,3,2-dioxaborolan-2-yl)phenyl)-5,6-dihydro-4H-cyclopenta[d]thiazol-4-ol as described in Intermediate 63, Step G. Data for (S) 4-(3-(4,4,5,5-tetramethyl-1,3,2-dioxaborolan-2-yl)phenyl)-5,6-dihydro-4H-cyclopenta[d]thiazol-4-ol: SFC Rₜ=3.52 min. LCMS (ESI): mass calcd. for C₁₈H₂₂BNO₃S 343.14 m/z, found 343.95 [M+H]⁺. ¹H-NMR (300 MHz, DMSO-d₆, ppm): δ 8.97 (s, 1H), 7.57-7.50 (m, 1H), 7.46-7.39 (m, 1H), 7.34-7.22 (m, 2H), 5.98 (s, 1H), 3.16-3.01 (m, 1H), 3.01-2.87 (m, 1H), 2.86-2.66 (m, 2H).

### Intermediate 103 (R) 4-(3-(4,4,5,5-Tetramethyl-1,3,2-dioxaborolan-2-yl)phenyl)-5,6-dihydro-4H-cyclopenta[d]thiazol-4-ol

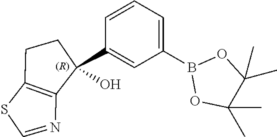

(R) 4-(3-(4,4,5,5-Tetramethyl-1,3,2-dioxaborolan-2-yl)phenyl)-5,6-dihydro-4H-cyclopenta[d]thiazol-4-ol, was obtained as a white solid, by the separation of the enantiomers of (R,S)-4-(3-(4,4,5,5-tetramethyl-1,3,2-dioxaborolan-2-yl)phenyl)-5,6-dihydro-4H-cyclopenta[d]thiazol-4-ol as described in Intermediate 63, Step G. Data for (R) 4-(3-(4,4,5,5-tetramethyl-1,3,2-dioxaborolan-2-yl)phenyl)-5,6-dihydro-4H-cyclopenta[d]thiazol-4-ol: Rₜ=2.95 min; LCMS (ESI), mass calcd. For C₁₈H₂₂BNO₃S 343.14 m/z, found 325.9 [M−H]⁺; ¹H-NMR (300 MHz, DMSO-d₆, ppm): d 8.97 (s, 1H), 7.57-7.50 (m, 1H), 7.46-7.40 (m, 1H), 7.34-7.22 (m, 2H), 5.98 (s, 1H), 3.16-3.01 (m, 1H), 3.01-2.87 (m, 1H), 2.86-2.66 (m, 2H).

### Intermediate 104. (S)-4-(3-(4-Bromothiazol-2-yl)phenyl)-5,6-dihydro-4H-cyclopenta[d]thiazol-4-ol

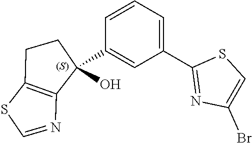

To a microwave tube containing (S)-4-(3-(4,4,5,5-tetramethyl-1,3,2-dioxaborolan-2-yl)phenyl)-5,6-dihydro-4H-cyclopenta[d]thiazol-4-ol (Intermediate 102, 700 mg, 2.0 mmol), 2,4-dibromothiazole (595 mg, 2.45 mmol) and K₂CO₃ (840 mg, 6.08 mmol) was added 1,4-dioxane:H₂O (4:1, 10 mL). The resultant mixture was sparged with N₂ for 5 minutes and then treated with Pd(dppf)Cl₂ (149 mg, 0.204 mmol). The resultant mixture was sparged with N₂ for another 5 minutes and then subjected to microwave irradiation at 90° C. for 1 h. After this time, the mixture was cooled to room temperature, diluted with H₂O (20 mL), and extracted with ethyl acetate (30 mL×3). The combined organic solvent extracts were washed with brine (50 mL), dried over anhydrous Na₂SO₄, filtered, concentrated and purified by FCC (eluent: petroleum ether:ethyl acetate=1:0 to 1:1) to yield (S)-4-(3-(4-bromothiazol-2-yl)phenyl)-5,6-dihydro-4H-cyclopenta[d]thiazol-4-ol (400 mg, 39%) as a yellow solid. ¹H NMR (400 MHz, CD₃OD) δ 8.48-8.44 (m, 1H), 8.12-8.05 (m, 2H), 7.83-7.78 (m, 1H), 7.59-7.53 (m, 1H), 7.01 (s, 1H), 3.60-3.52 (m, 2H), 2.95 (s, 3H), 2.79-2.68 (m, 1H), 2.45-2.32 (m, 1H).

### Intermediate 105. (S)-4-(3-(4-(1-(Phenylsulfonyl)-1H-pyrrolo[2,3-b]pyridin-3-yl)thiazol-2-yl)phenyl)-5,6-dihydro-4H-cyclopenta[d]thiazol-4-ol

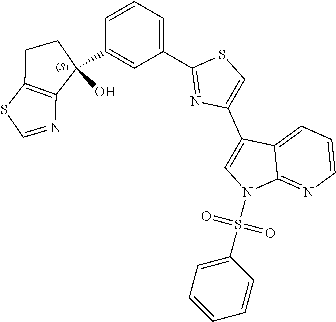

To a tube containing (S)-4-(3-(4-Bromothiazol-2-yl)phenyl)-5,6-dihydro-4H-cyclopenta[d]thiazol-4-ol (Intermediate 104, 400 mg, 1.06 mmol), 1-(phenylsulfonyl)-3-(4,4,5,5-tetramethyl-1,3,2-dioxaborolan-2-yl)-1H-pyrrolo[2,3-b]pyridine (487 mg, 1.27 mmol), K₂CO₃ (437 mg, 3.16 mmol) was added 1,4-dioxane/H₂O (10 mL, 4:1). The resultant mixture was sparged with N₂ for 5 minutes and then treated with Pd(dppf)Cl₂ (77 mg, 0.11 mmol). The resultant mixture was sparged with N₂ for another 5 minutes and then subjected to microwave irradiation at 90° C. for 1 h. After this time, the mixture was cooled to room temperature, diluted with H₂O (10 mL), and extracted with ethyl acetate (20 mL×3). The combined organic solvent extracts were washed with brine, dried over anhydrous Na₂SO₄, filtered, and concentrated to yield (S)-4-(3-(4-(1-(phenylsulfonyl)-1H-pyrrolo[2,3-b]pyridin-3-yl)thiazol-2-yl)phenyl)-5,6-dihydro-4H-cyclopenta[d]thiazol-4-ol (700 mg) as a black solid. LCMS (ESI): mass calcd. for C₂₈H₂₀N₄O₃S₃, 556.07: m/z found, 557.0 [M+H]⁺.

### Intermediate 106. (R)-4-(3-(4-Bromothiazol-2-yl)phenyl)-5,6-dihydro-4H-cyclopenta[d]thiazol-4-ol

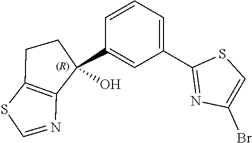

(R)-4-(3-(4-Bromothiazol-2-yl)phenyl)-5,6-dihydro-4H-cyclopenta[d]thiazol-4-ol was prepared in a manner analogous to (S)-4-(3-(4-Bromothiazol-2-yl)phenyl)-5,6-dihydro-4H-cyclopenta[d]thiazol-4-ol (Intermediate 104) using (R) 4-(3-(4,4,5,5-Tetramethyl-1,3,2-dioxaborolan-2-yl)phenyl)-5,6-dihydro-4H-cyclopenta[d]thiazol-4-ol (Intermediate 103) in place of (S)-4-(3-(4,4,5,5-tetramethyl-1,3,2-dioxaborolan-2-yl)phenyl)-5,6-dihydro-4H-cyclopenta[d]thiazol-4-ol (Intermediate 102). Data for (R)-4-(3-(4-bromothiazol-2-yl)phenyl)-5,6-dihydro-4H-cyclopenta[d]thiazol-4-ol: LCMS (ESI): mass calcd. for C₁₅H₁₁BrN₂OS₂, 377.95; m/z found, 362.7 [M−17]+.

### Intermediate 107. (R)-4-(3-(4-(1-(Phenylsutfonyl)-1H-pyrrolo[2,3-b]pyridin-3-yl)thiazol-2-yl)phenyl)-5,6-dihydro-4H-cyclopenta[d]thiazol-4-ol

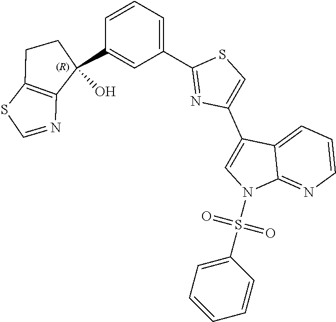

To a tube containing (R)-4-(3-(4-bromothiazol-2-yl)phenyl)-5,6-dihydro-4H-cyclopenta[d]thiazol-4-ol (Intermediate 106, 300 mg, 0.791 mmol), 1-(phenylsulfonyl)-3-(4,4,5,5-tetramethyl-1,3,2-dioxaborolan-2-yl)-1H-pyrrolo[2,3-b]pyridine (365 mg, 0.950 mmol), and K₂CO₃ (328 mg, 2.37 mmol) was added 1,4-dioxane/H₂O (10 mL, 4:1). The resultant mixture was sparged with N₂ for 5 minutes and then treated with Pd(dppf)Cl₂ (58 mg, 0.079 mmol). The resultant mixture was sparged with N₂ for another 5 minutes and then subjected to microwave irradiation at 90° C. for 1 h. After this time, the mixture was cooled to room temperature, diluted with H₂O (10 mL) and extracted with ethyl acetate (20 mL×3). The combined organic solvent extracts were washed with brine, dried over anhydrous Na₂SO₄, filtered, and concentrated under reduced pressure to yield (R)-4-(3-(4-(1-(phenylsulfonyl)-1H-pyrrolo[2,3-b]pyridin-3-yl)thiazol-2-yl)phenyl)-5,6-dihydro-4H-cyclopenta[d]thiazol-4-ol (700 mg) as a black solid, which was used without further purification.

### Intermediate 108. (R,S)-1-(3-Bromophenyl)-1-(pyridin-2-yl)ethan-1-ol

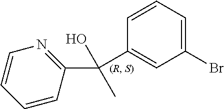

n-Butyllithium (16.5 mL, 41.3 mmol, 2.5 M in hexane) was added dropwise to a mixture of 1,3-dibromobenzene (6.0 mL, 50 mmol) and anhydrous THF (80 mL) that had been cooled to −72° C. The resultant mixture was stirred at −72° C. for 20 minutes and then treated with a mixture of 1-(pyridin-2-yl)ethanone (5.0 g, 41 mmol) in anhydrous THF (10 mL). The resultant mixture was stirred at −72° C. for 30 minutes. After this time, the reaction mixture was quenched with aqueous saturated NH₄Cl solution (50 mL), diluted with water (50 mL) and extracted with ethyl acetate (80 mL×3). The combined organic solvent extracts were washed with brine, dried over anhydrous Na₂SO₄, filtered, concentrated and purified by FCC (eluent: petroleum ether:ethyl acetate=1:0 to 9:1) to yield (R,S)-1-(3-bromophenyl)-1-(pyridin-2-yl)ethan-1-ol (7.9 g, 83% purity, 57% yield) as a yellow oil. LCMS (ESI): mass calcd. for C₁₃H₁₂BrNO, 277.01; m/z found, 277.9 [M+H]⁺.

### Intermediate 109. (R,S)-1-(Pyridin-2-yl)-1-(3-(4,4,5,5-tetramethyl-1,3,2-dioxaborolan-2-yl)phenyl)ethan-1-ol

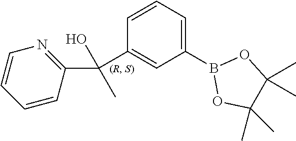

[1,1′-Bis(diphenylphosphino)ferrocene]dichloropalladium(II) (1.1 g, 1.5 mmol) was added to a mixture of (R,S)-1-(3-bromophenyl)-1-(pyridin-2-yl)ethanol (Intermediate 108, 4.9 g, 15 mmol, 83% purity), 4,4,4′,4′,5,5,5′,5′-octamethyl-2,2′-bi(1,3,2-dioxaborolane) (7.5 g, 30 mmol), KOAc (4.5 g, 46 mmol), and 1,4-dioxane (50 mL). The mixture was sparged with N₂ for 5 minutes, and then heated at 80° C. under N₂ for 16 h. After this time, the mixture was filtered through a pad of diatomaceous earth and the pad washed with ethyl acetate (50 mL). The filtrate was washed with water (50 mL). The aqueous phase was extracted again with ethyl acetate (50 mL×3). The combined organic solvent extracts were dried over anhydrous Na₂SO₄, filtered, concentrated and purified by FCC (eluent: petroleum ether:ethyl acetate=1:0 to 1:1) to yield (R,S)-1-(pyridin-2-yl)-1-(3-(4,4,5,5-tetramethyl-1,3,2-dioxaborolan-2-yl)phenyl)ethan-1-ol (3.4 g, 66% yield) as a yellow oil. LCMS (ESI): mass calcd. for C₁₉H₂₄BNO₃, 325.18; m/z found, 326.2 [M+H]⁺.

### Intermediate 110. (R,S)-1-(3-(2-(1-(Phenylsulfonyl)-1H-pyrrolo[2,3-b]pyridin-3-yl)thiazol-4-yl)phenyl)-1-(pyridin-2-yl)ethan-1-ol

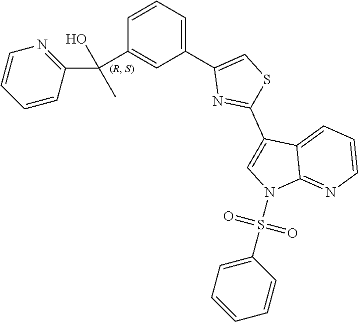

To a microwave tube containing (R,S)-1-(pyridin-2-yl)-1-(3-(4,4,5,5-tetramethyl-1,3,2-dioxaborolan-2-yl)phenyl)ethanol (Intermediate 109, 800 mg, 2.26 mmol), 4-bromo-2-(1-(phenylsulfonyl)-1H-pyrrolo[2,3-b]pyridin-3-yl)thiazole (Intermediate 2, 1.1 g, 2.6 mmol), and K₃PO₄ (1.5 g, 7.1 mmol) was added 1,4-dioxane/H₂O (10 mL, 4:1). The mixture was sparged with N₂ for 5 min and then Pd(dtbpf)Cl₂ (170 mg, 0.261 mmol) was added and the mixture was sparged with N₂ for another 5 minutes. The resultant mixture was subjected to microwave irradiation at 90° C. for 2 h. After this time, the mixture was cooled to room temperature, filtered through a pad of diatomaceous earth, and the pad was washed with ethyl acetate (30 mL). The filtrate was dried over anhydrous Na₂SO₄, filtered, and concentrated under reduced pressure to yield (R,S)-1-(3-(2-(1H-pyrrolo[2,3-b]pyridin-3-yl)thiazol-4-yl)phenyl)-1-(pyridin-2-yl)ethan-1-ol (2.0 g) as a red solid, which was used for next step without further purification. LCMS (ESI): mass calcd. for C₂₉H₂₂N₄O₃S₂, 538.11; m/z found, 539.1 [M+H]⁺.

### Intermediate 111. (S)-3-(3-bromophenyl)-3-hydroxy-1-methylpyrrolidin-2-one

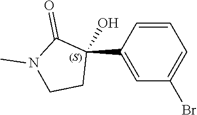

The chiral SFC separation described in Intermediate 15, Step 8 provided (S)-3-(3-bromophenyl)-3-hydroxy-1-methylpyrrolidin-2-one (1.56 g, 45%, SFC Rₜ=4.71 min) as a colorless solid.

### Intermediate 112. (R)-7-(3-(2-(5-tosyl-5H-pyrrolo[2,3-b]pyrazin-7-yl-6-d)thiazol-4-yl)phenyl)-6,7-dihydro-5H-pyrrolo[1,2-a]imidazol-7-ol

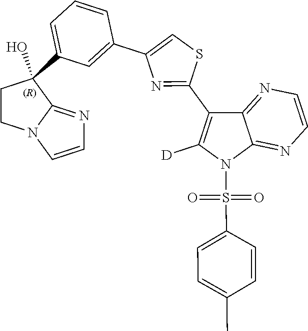

4-Bromo-2-(5-tosyl-5H-pyrrolo[2,3-b]pyrazin-7-yl-6-d)thiazole (Intermediate 41, 120 mg, 0.275 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 110 mg, 0.337 mmol), and K₃PO₄ (175 mg, 0.824 mmol) were added to a microwave tube and dissolved in 1,4-dioxane (1.6 mL) and H₂O (0.4 mL). The resultant mixture was sparged with Ar for 5 minutes and then treated with Pd(dtbpf)Cl₂ (19 mg, 0.029 mmol). This mixture was sparged with Ar for another 5 minutes and then subjected to microwave irradiation at 85° C. for 1 h. After this time, the mixture was cooled to room temperature, diluted with water (6 mL), and extracted with EtOAc/MeOH (9:1, 8 mL×5). The combined organic solvent extracts were dried over anhydrous Na₂SO₄, filtered, and concentrated under reduced pressure to afford (R)-7-(3-(2-(5-tosyl-5H-pyrrolo[2,3-b]pyrazin-7-yl-6-d)thiazol-4-yl)phenyl)-6,7-dihydro-5H-pyrrolo[1,2-a]imidazol-7-ol (190 mg, 90%) as a brown oil. LCMS(ESI): mass calcd. for C₂₈H₂₁DN₆O₃S₂, 555.13; m/z found, 556.1 [M+H]⁺.

### Intermediate 113. (S)-3-Hydroxy-3-(3-(4,4,5,5-tetramethyl-1,3,2-dioxaborolan-2-yl)phenyl)-1-(2,2,2-trifluoroethyl)pyrrolidin-2-one

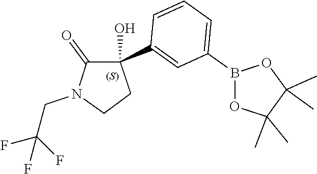

(S)-3-(3-Bromophenyl)-3-hydroxy-1-(2,2,2-trifluoroethyl)pyrrolidin-2-one (Intermediate 76, 90 mg, 0.27 mmol), 4,4,4′,4′,5,5,5′,5′-octamethyl-2,2′-bi(1,3,2-dioxaborolane) (108 mg, 0.425 mmol), KOAc (78 mg, 0.80 mmol), and 1,4-dioxane (10 mL) were added to a microwave tube. The mixture was sparged with N₂ for 5 minutes and then treated with Pd(dppf)Cl₂ (20 mg, 0.03 mmol). The resultant mixture was subjected to microwave irradiation at 90° C. for 1 h and then cooled to room temperature. The suspension was filtered through a pad of diatomaceous earth and the pad washed with ethyl acetate (30 mL). The filtrate was concentrated to dryness under reduced pressure to afford the product, which was purified by FCC (eluent: petroleum ether:ethyl acetate=10:1 to 1:1) to afford (S)-3-hydroxy-3-(3-(4,4,5,5-tetramethyl-1,3,2-dioxaborolan-2-yl)phenyl)-(105 mg, 91%) as a brown oil. LCMS (ESI), mass calcd. for C₁₈H₂₃BF₃NO₄, 385.17; m/z found, 368.2 [M-18+H]⁺.

### Intermediate 114. (R)-3-Hydroxy-1-methyl-3-(3-(2-(5-tosyl-5H-pyrrolo[2,3-b]pyrazin-7-yl)thiazol-4-yl)phenyl)piperidin-2-one

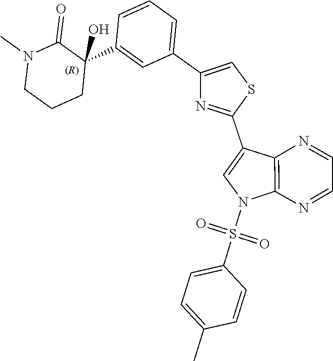

Step A. (R,S)-3-(3-bromophenyl)-3-hydroxy-1-methylpiperidin-2-one. n-Butyllithium (1.47 mL, 3.68 mmol, 2.5 M in hexane) was added dropwise to a solution of 1,3-dibromobenzene (868 mg, 3.68 mmol) and anhydrous THF (13 mL) that had been cooled to −70° C. The resultant mixture was stirred at −70° C. for 30 minutes and then treated with a solution of 1-methylpiperidine-2,3-dione (360 mg, 2.8 mmol) and anhydrous THF (2 mL). The reaction mixture was stirred at −50° C. for another 30 minutes. After this time, the mixture was quenched with aqueous saturated NH₄Cl solution (60 mL) and extracted with ethyl acetate (50 mL×3). The combined organic solvent extracts were dried over anhydrous Na₂SO₄, filtered, and concentrated to dryness under reduced pressure to afford the product, which was purified by FCC (eluent: petroleum ether:ethyl acetate=10:1 to 1:1) to afford (R,S)-3-(3-bromophenyl)-3-hydroxy-1-methylpiperidin-2-one (320 mg, 35%) as a brown solid. LCMS (ESI): mass calcd. for C₁₂H₁₄BrNO₂, 283.02; m/z found, 284.0 [M+1]⁺.

Step B. (R)-3-(3-bromophenyl)-3-hydroxy-1-methylpiperidin-2-one. The enantiomers of (R,S)-3-(3-bromophenyl)-3-hydroxy-1-methylpiperidin-2-one (320 mg, 1.1 mmol) were separated by SFC using an SFC column, such as a DAICEL CHIRALPAK AD 250 mm×30 mm×10 mm column (isocratic elution of EtOH (containing 0.1% of 25% aq. NH₃): supercritical CO₂=45%: 55%). The first eluting enantiomer was (R)-3-(3-bromophenyl)-3-hydroxy-1-methylpiperidin-2-one (140 mg, 44%, a white solid). The second eluting enantiomer was (S)-3-(3-bromophenyl)-3-hydroxy-1-methylpiperidin-2-one (140 mg, 44%, a white solid).

Step C. (R)-3-hydroxy-1-methyl-3-(3-(4,4,5,5-tetramethyl-1,3,2-dioxaborolan-2-yl)phenyl)piperidin-2-one. (R)-3-(3-Bromophenyl)-3-hydroxy-1-methylpiperidin-2-one (130 mg, 0.458 mmol), 4,4,4′,4′,5,5,5′,5′-octamethyl-2,2′-bi(1,3,2-dioxaborolane) (186 mg, 0.732 mmol), KOAc (135 mg, 1.38 mmol) and 1,4-dioxane (5 mL) were added to a microwave tube. The mixture was sparged with N₂ for 5 minutes and then treated with Pd(dppf)Cl₂ (33 mg, 0.05 mmol). The mixture was then subjected to microwave irradiation at 80° C. for 1 h. The reaction mixture was cooled to room temperature and the suspension was filtered through a pad of diatomaceous earth and the pad washed with ethyl acetate (60 mL). The filtrate was concentrated to dryness under reduced pressure to afford the product, which was purified by FCC (eluent: petroleum ether:ethyl acetate=10:1 to 1:1) to afford (R)-3-hydroxy-1-methyl-3-(3-(4,4,5,5-tetramethyl-1,3,2-dioxaborolan-2-yl)phenyl)piperidin-2-one (130 mg, 86%) as a brown oil. LCMS (ESI): mass calcd. for C₁₈H₂₆BNO₄, 331.20; m/z found, 332.3 [M+1]⁺.

Step D. (R)-3-hydroxy-1-methyl-3-(3-(2-(5-tosyl-5H-pyrrolo[2,3-b]pyrazin-7-yl)thiazol-4-yl)phenyl)piperidin-2-one. To a microwave tube containing 4-bromo-2-(5-tosyl-5H-pyrrolo[2,3-b]pyrazin-7-yl)thiazole (Intermediate 10, 157 mg, 0.361 mmol), (R)-3-hydroxy-1-methyl-3-(3-(4,4,5,5-tetramethyl-1,3,2-dioxaborolan-2-yl)phenyl)piperidin-2-one (120 mg, 0.362 mmol) and K₃PO₄ (231 mg, 1.09 mmol) was added 1,4-dioxane (10 mL) and H₂O (2 mL). The mixture was sparged with Ar for 5 minutes and then treated with Pd(dtbpf)Cl₂ (24 mg, 0.04 mmol). The mixture was sparged with Ar for another 5 minutes and then subjected to microwave irradiation at 80° C. for 1 h. After this time, the mixture was cooled to room temperature, poured into water (80 mL) and extracted with ethyl acetate (60 mL×3). The combined organic solvent extracts were dried over anhydrous Na₂SO₄, filtered, and concentrated to dryness under reduced pressure to afford the product, which was purified by FCC (eluent: petroleum ether:ethyl acetate=10:1 to 0:1) to give (R)-3-hydroxy-1-methyl-3-(3-(2-(5-tosyl-5H-pyrrolo[2,3-b]pyrazin-7-yl)thiazol-4-yl)phenyl)piperidin-2-one (130 mg, 63%) as a white solid. LCMS (ESI): mass calcd. for C₂₈H₂₅N₅O₄S₂, 559.13; m/z found, 560.2 [M+1]⁺.

### Intermediate 115. (S)-3-Hydroxy-1-methyl-3-(3-(2-(5-tosyl-5H-pyrrolo[2,3-b]pyrazin-7-yl)thiazol-4-yl)phenyl)piperidin-2-one

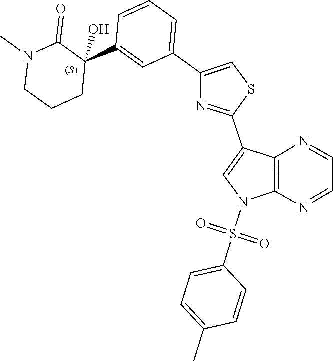

Step A. (S)-3-Hydroxy-1-methyl-3-(3-(4,4,5,5-tetramethyl-1,3,2-dioxaborolan-2-yl)phenyl)piperidin-2-one. (S)-3-(3-Bromophenyl)-3-hydroxy-1-methylpiperidin-2-one (Intermediate 114, Step B, 140 mg, 0.493 mmol) was added to a mixture of 4,4,4′,4′,5,5,5′,5′-octamethyl-2,2′-bi(1,3,2-dioxaborolane) (200 mg, 0.788 mmol), KOAc (145 mg, 1.48 mmol), and 1,4-dioxane (10 mL). The mixture was sparged with Ar for 5 minutes and then treated with Pd(dppf)Cl₂ (36 mg, 0.049 mmol). This mixture was sparged with Ar for another 5 minutes and then subjected to microwave irradiation at 85° C. for 1 h. After this time, the mixture was cooled to room temperature and 4,4,4′,4′,5,5,5′,5′-octamethyl-2,2′-bi(1,3,2-dioxaborolane) (100 mg, 0.394 mmol) and Pd(dppf)Cl₂ (18 mg, 0.025 mmol) were added to the reaction mixture. The mixture was sparged with Ar for another 5 minutes and then subjected to microwave irradiation at 85° C. for 1 h. After this time, the mixture was cooled to room temperature, filtered through a pad of diatomaceous earth and the pad washed with ethyl acetate (20 mL). The filtrate was concentrated to dryness under reduced pressure to afford the product, which was purified by FCC (eluent: petroleum ether ethyl acetate=1:0 to 3:2) to afford (S)-3-hydroxy-1-methyl-3-(3-(4,4,5,5-tetramethyl-1,3,2-dioxaborolan-2-yl)phenyl)piperidin-2-one (140 mg, 65%) as a brown oil. LCMS(ESI): mass calcd. for C₁₈H₂₈BNO₄, 331.20; m/z found, 332.3[M+H]⁺.

Step B. (S)-3-Hydroxy-1-methyl-3-(3-(2-(5-tosyl-5H-pyrrolo[2,3-b]pyrazin-7-yl)thiazol-4-yl)phenyl)piperidin-2-one. To a microwave tube containing 4-bromo-2-(5-tosyl-5H-pyrrolo[2,3-b]pyrazin-7-yl)thiazole (Intermediate 10, 187 mg, 0.430 mmol), (S)-3-hydroxy-1-methyl-3-(3-(4,4,5,5-tetramethyl-1,3,2-dioxaborolan-2-yl)phenyl)piperidin-2-one (140 mg, 0.423 mmol) and K₃PO₄ (266 mg, 1.25 mmol) was added 1,4-dioxane (12 mL) and H₂O (3 mL). The resultant mixture was sparged with Ar for 5 minutes and then treated with Pd(dtbpf)Cl₂ (31 mg, 0.048 mmol). This mixture was sparged with Ar for another 5 minutes and then subjected to microwave irradiation at 80° C. for 1 h. After this time, the mixture was cooled to room temperature, filtered through a pad of diatomaceous earth and the pad washed with ethyl acetate (30 mL). The filtrate was diluted with H₂O (20 mL) and extracted ethyl acetate (20 mL×3). The combined organic solvent extracts were washed with brine, dried over anhydrous Na₂SO₄, filtered, and concentrated to dryness under reduced pressure to afford the product, which was purified by FCC (eluent: petroleum ether:ethyl acetate=1:0 to 0:1) to afford (S)-3-hydroxy-1-methyl-3-(3-(2-(5-tosyl-5H-pyrrolo[2,3-b]pyrazin-7-yl)thiazol-4-yl)phenyl)piperidin-2-one (130 mg, 52%) as a brown oil. LCMS(ESI): mass calcd. for C₂₈H₂₅N₅O₄S₂, 559.13; m/z found, 560.1 [M+H]⁺.

### Intermediate 116. (S)-3-Hydroxy-1-methyl-3-(3-(4,4,5,5-tetramethyl-1,3,2-dioxaborolan-2-yl)phenyl)pyrrolidin-2-one

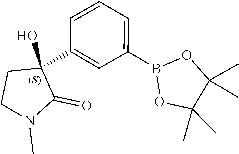

(S)-3-Hydroxy-1-methyl-3-(3-(4, 4, 5,5-tetramethyl-1, 3,2-dioxaborolan-2-yl)phenyl)pyrrolidin-2-one was prepared in analogous manner to Intermediate 4, except (S)-3-(3-bromophenyl)-3-hydroxy-1-methylpyrrolidin-2-one (Intermediate 111) was used in place of (R,S)-3-(3-bromophenyl)-3-hydroxy-1-methylpyrrolidin-2-one. LCMS (ESI): mass calcd. for C₁₇H₂₄BNO₄, 317.18; m/z found, 318.1 [M+1]⁺.

### Intermediate 117. (R)-7-(3-(2-(5-Tosyl-5H-pyrrolo[2,3-b]pyrazin-7-yl-2-d)thiazol-4-yl)phenyl)-6,7-dihydro-5H-pyrrolo[1,2-a]imidazol-7-ol

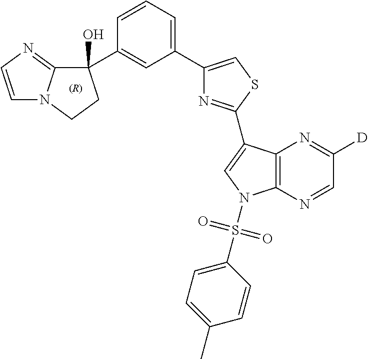

To a microwave tube containing 4-bromo-2-(5-tosyl-5H-pyrrolo[2,3-b]pyrazin-7-yl-2-d)thiazole (Intermediate 44, 140 mg, 0.320 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 105 mg, 0.320 mmol), and K₃PO₄ (200 mg, 0.94 mmol) was added 1,4-dioxane (12 mL) and H₂O (3 mL). The mixture was sparged with Ar for 5 minutes and then Pd(dtbpf)Cl₂ (21 mg, 0.03 mmol) was added. The mixture was sparged with Ar for another 5 minutes and the resultant mixture was heated at 80° C. in microwave for 1 h. The reaction mixture was cooled to room temperature, concentrated to dryness, and purified by FCC (petroleum ether:ethyl acetate=10:1 to 0:1, then ethyl acetate:methanol=1:0 to 20:1) to yield (R)-7-(3-(2-(5-tosyl-5H-pyrrolo[2,3-b]pyrazin-7-yl-2-d)thiazol-4-yl)phenyl)-6,7-dihydro-5H-pyrrolo[1,2-a]imidazol-7-ol (140 mg, 73%) as a yellow solid. LCMS (ESI): mass calcd. for C₂₈H₂₁DN₆O₃S₂, 555.13; m/z found, 556.1 [M+H]⁺.

### Intermediate 118. (S)-3-Hydroxy-3-(3-(2-(5-tosyl-5H-pyrrolo[2,3-b]pyrazin-7-yl)thiazol-4-yl)phenyl)-1-(2,2,2-trifluoroethyl)pyrrolidin-2-one

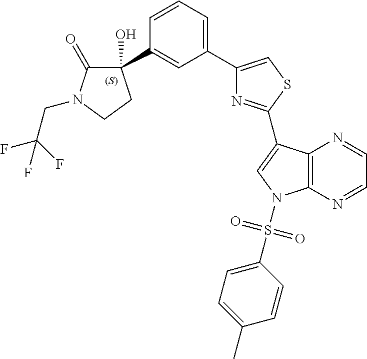

(S)-3-Hydroxy-3-(3-(2-(5-tosyl-5H-pyrrolo[2,3-b]pyrazin-7-yl)thiazol-4-yl)phenyl)-1-(2,2,2-trifluoroethyl)pyrrolidin-2-one was prepared in analogous manner to Intermediate 79, except (S)-3-hydroxy-3-(3-(4,4,5,5-tetramethyl-1,3,2-dioxaborolan-2-yl)phenyl)-1-(2,2,2-trifluoroethyl)pyrrolidin-2-one (Intermediate 113) was used instead of (R)-3-hydroxy-3-(3-(4,4,5,5-tetramethyl-1,3,2-dioxaborolan-2-yl)phenyl)-1-(2,2,2-trifluoroethyl)pyrrolidin-2-one (Intermediate 78). Data for (S)-3-hydroxy-3-(3-(2-(5-tosyl-5H-pyrrolo[2,3-b]pyrazin-7-yl)thiazol-4-yl)phenyl)-1-(2,2,2-trifluoroethyl)pyrrolidin-2-one: LCMS(ESI): mass calcd. for C₂₈H₂₂F₃N₅O₄S₂, 613.11; m/z found, 614.2 [M+H]⁺.

### Intermediate 119. (S)-7-(3-(2-(5-Tosyl-5H-pyrrolo[2,3-b]pyrazin-7-yl)thiazol-4-yl)phenyl)-6,7-dihydro-5H-pyrrolo[1,2-a]imidazol-7-ol

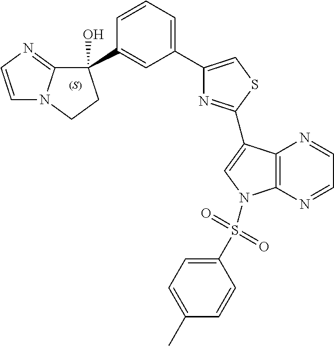

(S)-7-(3-(2-(5-tosyl-5H-pyrrolo[2,3-b]pyrazin-7-yl)thiazol-4-yl)phenyl)-6,7-dihydro-5H-pyrrolo[1,2-a]imidazol-7-ol was prepared in a manner analogous to Intermediate 14 using (S)-7-(3-(4,4,5,5-tetramethyl-1,3,2-dioxaborolan-2-yl)phenyl)-6,7-dihydro-5H-pyrrolo[1,2-a]imidazol-7-ol (Intermediate 39) instead of (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13). Data for (S)-7-(3-(2-(5-tosyl-5H-pyrrolo[2,3-b]pyrazin-7-yl)thiazol-4-yl)phenyl)-6,7-dihydro-5H-pyrrolo[1,2-a]imidazol-7-ol: LCMS(ESI): mass calcd. for C₂₈H₂₂N₆O₃S₂, 554.12; m/z found, 555.10 [M+H]⁺.

### Intermediate 120.1-(3-(2-(1-(phenylsulfonyl)-1H-pyrrolo[2,3-b]pyridin-3-yl)thiazol-4-yl)phenyl)-1-(thiazol-2-yl)ethan-1-ol

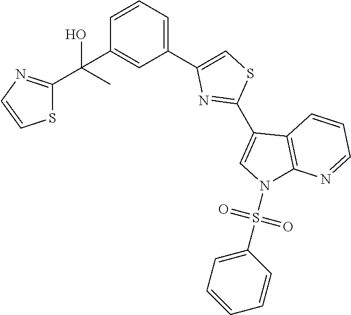 [SMILES: CC(O)(c1cccc(-c2csc(-c3cn(S(=O)(=O)c4ccccc4)c4ncccc34)n2)c1)c1nccs1]

Step A. 1-(3-Bromophenyl)-1-(thiazol-2-yl)ethanol. Isopropylmagnesium chloride (11.6 mL, 2.0 M in THF, 23.17 mmol) was added dropwise to a 0° C. solution of 2-bromothiazole (4.0 g, 24 mmol) in anhydrous THF (25 mL). The resultant mixture was then cooled to −78° C. (dry ice/ethanol) and treated with 1-(3-bromophenyl)ethanone (4.6 g in 5 mL THF, 23 mmol). The resultant mixture was stirred at −78° C. for 30 minutes and then stirred for 2 h with gradual warming to room temperature. After this time, the mixture was quenched with saturated NH₄Cl (20 mL), diluted with H₂O (10 mL), and extracted with ethyl acetate (30 mL). The organic extract was dried over anhydrous Na₂SO₄, filtered, and concentrated to dryness under reduced pressure to give the product, which was purified by silica gel chromatography (eluent: petroleum ether ethyl acetate=1:0 to 3:1) to afford the title compound (2.91 g, 35%) as a yellow oil. LC-MS (ESI): mass calcd. for C₁₁H₁₀BrNOS 284.96 m/z, found 286.0 [M+H]⁺.

Step B. 1-(3-(4,4,5,5-Tetramethyl-1,3,2-dioxaborolan-2-yl)phenyl)-1-(thiazol-2-yl)ethanol. 1-(3-Bromophenyl)-1-(thiazol-2-yl)ethanol (2.91 g, 8.50 mmol), bis(pinacolato)diboron (3.24 g, 12.7 mmol), and KOAc (2.50 g, 25.5 mmol) were dissolved in 1,4-dioxane (30 mL). The resultant mixture was sparged with N₂ for 5 minutes and then treated with Pd(dppf)Cl₂ (0.62 g, 0.85 mmol). The mixture was sparged with N₂ for another 5 minutes and then stirred at 100° C. for 16 hours. After this time, the mixture was cooled room temperature, poured into water (15 mL), and extracted with ethyl acetate (15 mL×2). The combined organic extracts were washed with brine (15 mL), dried over anhydrous Na₂SO₄, filtered, and concentrated to dryness under reduced pressure. to give the product, which was purified by silica gel chromatography (eluent: petroleum ether:ethyl acetate=1:0 to 3:1) to afford the title compound (1.93 g, 63%) as a yellow oil. LC-MS (ESI): mass calcd. for C₁₇H₂₂BNO₃S 331.14 m/z, found 332.2 [M+H]⁺.

Step C. 1-(3-(2-(1-(Phenylsulfonyl)-1H-pyrrolo[2,3-b]pyridin-3-yl)thiazol-4-yl)phenyl)-1-(thiazol-2-yl)ethanol. 4-Bromo-2-(1-(phenylsulfonyl)-1H-pyrrolo[2,3-b]pyridin-3-yl)thiazole (Intermediate 2, 400 mg, 0.952 mmol), 1-(3-(4,4,5,5-tetramethyl-1,3,2-dioxaborolan-2-yl)phenyl)-1-(thiazol-2-yl)ethanol (435 mg, 1.14 mmol), and K₃PO₄ (606 mg, 2.86 mmol) were dissolved in 1,4-dioxane (12 mL) and H₂O (3 mL). The mixture was sparged with N₂ for 5 minutes and then treated with Pd(dtbpf)Cl₂ (62 mg, 0.095 mmol). The resultant mixture was heated at 80° C. via microwave irradiation for 1 hour. After this time, the mixture was cooled to room temperature and filtered through a pad of diatomaceous earth. The filtrate was concentrated under reduced pressure to afford the product, which was purified by silica gel chromatography (eluent: petroleum ether:ethyl acetate=1:0 to 1:1) which afforded the title compound (250 mg, 47%) as a yellow solid. LC-MS (ESI): mass calcd. for C₂₇H₂₀N₄O₃S₃ 544.07 m/z, found 545.1 [M+H]⁺. ¹H NMR (400 MHz, DMSO-d₆) δ 8.74-8.69 (m, 1H), 8.67 (s, 1H), 8.54-8.49 (m, 1H), 8.24 (s, 2H), 8.22 (s, 1H), 8.16 (s, 1H), 7.92 (d, J=7.6 Hz, 1H), 7.79-7.72 (m, 2H), 7.69-7.61 (m, 3H), 7.61-7.53 (m, 2H), 7.44 (t, J=7.6 Hz, 1H), 6.81 (s, 1H), 1.98 (s, 3H).

Step D. 1-(3-(2-(1H-Pyrrolo[2,3-b]pyridin-3-yl)thiazol-4-yl)phenyl)-1-(thiazol-2-yl)ethanol. Lithium hydroxide (0.673 mL, 2.02 mmol, 3.0 M in H₂O) was added to a solution of 1-(3-(2-(1-(phenylsulfonyl)-1H-pyrrolo[2,3-b]pyridin-3-yl)thiazol-4-yl)phenyl)-1-(thiazol-2-yl)ethanol (220 mg, 0.404 mmol) and 1,4-dioxane (10 mL). The resultant mixture was heated at 70° C. for 2 hours. After this time, the mixture was cooled to room temperature and concentrated to dryness under reduced pressure to give the product, which was purified by preparative HPLC over a Phenomenex Gemini 150×25 mm×10 μm column (eluent: 40% to 70% (v/v) CH₃CN and H₂O with 0.04% NH₃). The pure fractions were combined, and the volatiles were removed under vacuum. The residue was extracted with ethyl acetate (50 mL×2). The combined organic extracts were dried over Na₂SO₄, filtered, and concentrated to dryness under reduced pressure to afford the title compound (75 mg, 43%) as a yellow solid. LC-MS (ESI): mass calcd. for C₂₁H₁₆N₄O S₂ 404.08 m/z, found 405.0 [M+H]⁺.

### Intermediate 121. (R)-3-hydroxy-1-methyl-3-(3-(5-methyl-2-(5-tosyl-5H-pyrrolo[2,3-b]pyrazin-7-yl)thiazol-4-yl)phenyl)pyrrolidin-2-one

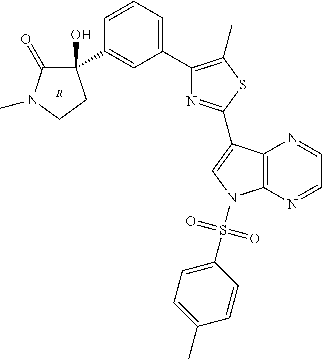

Step A. 4-Bromo-5-methyl-2-(5-tosyl-5H-pyrrolo[2,3-b]pyrazin-7-yl)thiazole. 7-(4,4,5,5-Tetramethyl-1,3,2-dioxaborolan-2-yl)-5-tosyl-5H-pyrrolo[2,3-b]pyrazine (500 mg, 1.25 mmol), 2,4-dibromo-5-methylthiazole (386 mg, 1.50 mmol), Na₂CO₃ (398 mg, 3.76 mmol), and DMF (5 mL) were added to a 20 mL microwave tube. The resultant mixture was sparged with N₂ for 5 minutes and then treated with Pd(PPh₃)₂Cl₂ (88 mg, 0.13 mmol). The resultant mixture was sparged with N₂ for another 5 minutes and then heated to 80° C. via microwave irradiation for 1 hour. After this time, the mixture was cooled to room temperature, filtered through a pad of diatomaceous earth and the pad was washed with ethyl acetate (15 mL). The filtrate was concentrated to dryness under reduced pressure to give the product, which was purified by prep. HPLC using a Boston Prime C18 150×30 mm×5 μm column (eluent: 30% to 60% (v/v) CH₃CN and H₂O with (0.04% NH₃H₂O+10 mM NH₄HCO₃)) to afford the title compound (100 mg, 28%) as a colorless solid. LCMS (ESI): mass calcd. for C₁₇H₁₃BrN₄O₂S₂ 449.35 m/z, found 451.0 [M+H]⁺.

Step B. (R)-3-Hydroxy-1-methyl-3-(3-(5-methyl-2-(5-tosyl-5H-pyrrolo[2,3-b]pyrazin-7-yl)thiazol-4-yl)phenyl)pyrrolidin-2-one. 4-Bromo-5-methyl-2-(5-tosyl-5H-pyrrolo[2,3-b]pyrazin-7-yl)thiazole (50 mg, 0.11 mmol), (R)-3-hydroxy-1-methyl-3-(3-(4,4,5,5-tetramethyl-1,3,2-dioxaborolan-2-yl)phenyl)pyrrolidin-2-one (Intermediate 16, 35 mg, 0.11 mmol), K₃PO₄ (71 mg, 0.33 mmol), 1,4-dioxane (2 mL), and H₂O (0.5 mL) were added to a microwave tube. The resultant mixture was sparged with Ar for 5 minutes and then treated with Pd(dtbpf)Cl₂ (7 mg, 0.01 mmol). The resultant mixture was sparged with Ar for another 5 minutes and then heated to 80° C. via microwave irradiation for 1 hour. After this time, the mixture was cooled to room temperature. The reaction mixture was filtered through a pad of diatomaceous earth and the pad was washed with ethyl acetate (15 mL). The filtrate was concentrated to dryness under reduced pressure to give the product, which was purified by prep. HPLC using a Boston Prime C18 250×50 mm×10 μm column (eluent: 50% to 80% (v/v) CH₃CN and H₂O with 0.04% NH₃H₂O+10 mM NH₄HCO₃) to afford the title compound (50 mg, 80%) as a colorless solid. LCMS (ESI): mass calcd. for C₂₈H₂₅N₅O₄S₂ 559.13 m/z, found 560.2 [M+H]⁺. ¹H NMR (400 MHz, DMSO-d₆) δ 8.78-8.74 (m, 1H), 8.70 (s, 1H), 8.58-8.54 (m, 1H), 8.10 (d, J=8.3 Hz, 2H), 7.77 (s, 1H), 7.68-7.63 (m, 1H), 7.48-7.40 (m, 3H), 7.37-7.31 (m, 1H), 6.09 (s, 1H), 3.53 (5, 3H), 3.48-3.40 (m, 1H), 3.38-3.34 (m, 1H), 2.84 (s, 3H), 2.60 (s, 3H), 2.32 (s, 2H).

### Intermediate 122. (S)-3-hydroxy-1-methyl-3-(3-(5-methyl-2-(5-tosyl-5H-pyrrolo[2,3-b]pyrazin-7-yl)thiazol-4-yl)phenyl)pyrrolidin-2-one

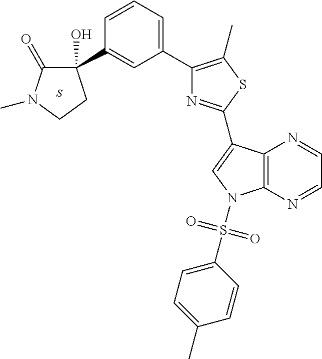

To a microwave vial containing 4-Bromo-5-methyl-2-(5-tosyl-5H-pyrrolo[2,3-b]pyrazin-7-yl)thiazole (142 mg, 0.316 mmol), (S)-3-hydroxy-1-methyl-3-(3-(4,4,5,5-tetramethyl-1,3,2-dioxaborolan-2-yl)phenyl)pyrrolidin-2-one (Intermediate 116, 100 mg, 0.315 mmol) and K₃PO₄ (201 mg, 0.947 mmol) was added 1,4-dioxane (4 mL), and H₂O (1 mL). The mixture was sparged with Ar for 5 minutes and then treated with Pd(dtbpf)Cl₂ (21 mg, 0.030 mmol). The resultant mixture was sparged with Ar for another 5 minutes and then heated at 80° C. via microwave irradiation for 1 hour. After this time, the mixture was cooled to room temperature, filtered through a pad of diatomaceous earth, and the pad was washed with ethyl acetate (15 mL). The filtrate was concentrated to dryness under reduced pressure to give the product (400 mg) as a brown solid. LCMS (ESI): mass calcd. for C₂₈H₂₅N₅O₄S₂ 559.13 m/z, found 560.2 [M+H]⁺.

### Intermediate 123. 3-(3-bromo-4-methylphenyl)-3-hydroxy-1-methylpyrrolidin-2-one

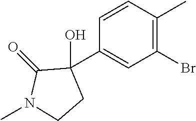 [SMILES: Cc1ccc(C2(O)CCN(C)C2=O)cc1Br]

n-BuLi (2.3 mL, 2.5 M in hexane, 5.8 mmol) was added drop-wise to a −70° C. (dry ice/EtOH) solution of 2,4-dibromo-1-methylbenzene (1.4 g, 5.6 mmol) in THE (20 mL). The resultant mixture was stirred at −70° C. for 30 min before treating with 1-methylpyrrolidine-2,3-dione (500 mg, 4.42 mmol). The mixture was stirred at −50° C. for min and then quenched with sat. NH₄Cl (20 mL) and extracted with ethyl acetate (20 mL×3). The combined organic extracts were dried over anhydrous Na₂SO₄, filtered, and concentrated under reduced pressure to afford the product, which was purified by silica gel chromatography (petroleum ether:ethyl acetate=10:1 to 0:1) to afford the title compound as a mixture with 3-(5-bromo-2-methylphenyl)-3-hydroxy-1-methylpyrrolidin-2-one (600 mg), as a light yellow solid. Data for 3-(3-bromo-4-methylphenyl)-3-hydroxy-1-methylpyrrolidin-2-one: LCMS (ESI): mass calcd. for C₁₂H₁₄BrNO₂ 283.02 m/z, found 284.14 [M+H]⁺.

### Intermediate 124. 3-(5-bromo-2-methylphenyl)-3-hydroxy-1-methylpyrrolidin-2-one

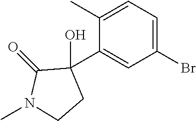 [SMILES: Cc1ccc(Br)cc1C1(O)CCN(C)C1=O]

The title compound was obtained as a mixture with 3-(3-bromo-4-methylphenyl)-3-hydroxy-1-methylpyrrolidin-2-one. LCMS (ESI): mass calcd. for C₁₂H₁₄BrNO₂ 283.02 m/z, found 284.14 [M+H]⁺.

### Intermediate 125. (R)-3-(3-bromo-4-methylphenyl)-3-hydroxy-1-methylpyrrolidin-2-one

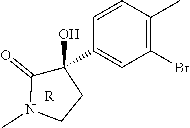

A mixture of 3-(3-bromo-4-methylphenyl)-3-hydroxy-1-methylpyrrolidin-2-one (Intermediate 123) and 3-(5-bromo-2-methylphenyl)-3-hydroxy-1-methylpyrrolidin-2-one (Intermediate 124), (600 mg) was separated and purified by SFC over DAICEL CHIRALPAK AS-H 250 mm×30 mm, 5 μm (isocratic elution: EtOH, containing 0.1% of 25% aq. NH₃): supercritical CO₂, 25%: 75% to 25%: 75% (v/v)) to afford 4 products: (R)-3-(3-bromo-4-methylphenyl)-3-hydroxy-1-methylpyrrolidin-2-one (Intermediate 125), (S) 3-(3-bromo-4-methylphenyl)-3-hydroxy-1-methylpyrrolidin-2-one (Intermediate 126), (R)-3-(5-bromo-2-methylphenyl)-3-hydroxy-1-methylpyrrolidin-2-one (Intermediate 127), (S)-3-(5-bromo-2-methylphenyl)-3-hydroxy-1-methylpyrrolidin-2-one (Intermediate 128). Data for (R)-3-(3-bromo-4-methylphenyl)-3-hydroxy-1-methylpyrrolidin-2-one (Intermediate 125) (170 mg, white solid). ¹H NMR (400 MHz, CDCl₃) δ 7.37-7.35 (m, 1H), 7.33-7.29 (m, 1H), 7.05 (d, J=8.0 Hz, 1H), 3.50-3.42 (m, 1H), 3.35-3.27 (m, 1H), 3.03 (s, 3H), 2.48-2.38 (m, 2H), 2.37 (s, 3H).

### Intermediate 126. (S)-3-(3-bromo-4-methylphenyl)-3-hydroxy-1-methylpyrrolidin-2-one

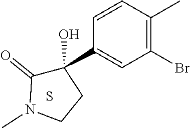

The chiral separation described in Intermediate 125 provided the title compound (190 mg) as a white solid. ¹H NMR (400 MHz, CDCl₃) δ 7.36 (d, J=2.3 Hz, 1H), 7.31 (dd, J=2.3, 8.0 Hz, 1H), 7.05 (d, J=8.0 Hz, 1H), 3.50-3.42 (m, 1H), 3.35-3.27 (m, 1H), 3.03 (s, 3H), 2.44-2.37 (m, 2H), 2.37 (s, 3H).

### Intermediate 127. (R)-3-(5-bromo-2-methylphenyl)-3-hydroxy-1-methylpyrrolidin-2-one

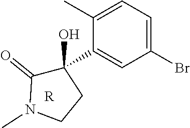

The chiral separation described in Intermediate 125 provided the title compound (115 mg) as a white solid. ¹H NMR (400 MHz, CDCl₃) δ 7.57 (s, 1H), 7.22-7.19 (m, 2H), 3.47-3.25 (m, 3H), 3.01 (s, 3H), 2.51-2.42 (m, 1H), 2.38 (s, 3H), 2.37-2.33 (m, 1H).

### Intermediate 128. (S)-3-(5-bromo-2-methylphenyl)-3-hydroxy-1-methylpyrrolidin-2-one

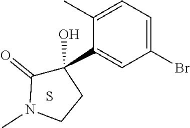

The chiral separation described in Intermediate 125 provided the title compound (110 mg) as a white solid. ¹H NMR (400 MHz, CDCl₃) δ 7.56 (s, 1H), 7.21-7.18 (m, 2H), 3.49-3.34 (m, 3H), 3.00 (s, 3H), 2.50-2.42 (m, 1H), 2.38 (s, 3H), 2.37-2.31 (m, 1H).

### Intermediate 129. (S)-3-hydroxy-1-methyl-3-(4-methyl-3-(4,4,5,5-tetramethyl-1,3,2-dioxaborolan-2-yl)phenyl)pyrrolidin-2-one

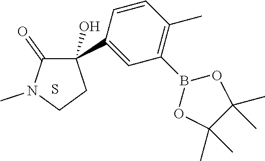

To a microwave vial containing (S)-3-(3-Bromo-4-methylphenyl)-3-hydroxy-1-methylpyrrolidin-2-one (Intermediate 126, 190 mg, 0.669 mmol), 4,4,4′,4′,5,5,5′,5′-octamethyl-2,2′-bi(1,3,2-dioxaborolane) (340 mg, 1.34 mmol), and KOAc (197 mg, 2.01 mmol) was added 1,4-dioxane (10 mL). The mixture was sparged with N₂ for 5 minutes and then treated with Pd(dppf)Cl₂ (49 mg, 0.067 mmol). The resultant mixture was heated to 85° C. via microwave irradiation for 1 hour and then cooled to room temperature. The suspension was filtered through a pad of diatomaceous earth and then the filtrate was concentrated under reduced pressure to give the product, which was purified by silica gel chromatography (eluent: petroleum ether:ethyl acetate=1:0 to 0:1) to afford the title compound (200 mg, 86% purity, 78%) as a brown oil. LCMS (ESI): mass calcd. for C₁₈H₂₈BNO₄ 331.21 m/z, found 332.0 [M+H]⁺.

### Intermediate 130. (R)-3-hydroxy-1-methyl-3-(4-methyl-3-(2-(5-tosyl-5H-pyrrolo[2,3-b]pyrazin-7-yl)thiazol-4-yl)phenyl)pyrrolidin-2-one

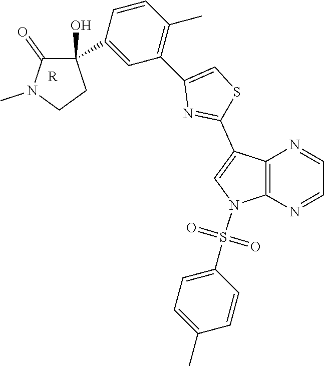

To a microwave vial containing (R)-3-Hydroxy-1-methyl-3-(4-methyl-3-(4,4,5,5-tetramethyl-1,3,2-dioxaborolan-2-yl)phenyl)pyrrolidin-2-one (200 mg, 0.604 mmol), 4-bromo-2-(5-tosyl-5H-pyrrolo[2,3-b]pyrazin-7-yl)thiazole (Intermediate 10, 158 mg, 0.363 mmol), and K₃PO₄ (385 mg, 1.81 mmol), was added 1,4-dioxane (8 mL) and H₂O (2 mL). The mixture was sparged with Ar for 5 minutes and then treated with Pd(dtbpf)Cl₂ (39 mg, 0.060 mmol). The mixture was sparged again with Ar for another 5 minutes and then heated at 80° C. via microwave irradiation for 1 hour. The reaction mixture was cooled to room temperature, and then filtered through a pad of diatomaceous earth and the pad was washed with ethyl acetate (30 mL). The filtrate was poured into H₂O (20 mL) and extracted ethyl acetate (20 mL×3). The combined organic extracts were washed with brine, dried over anhydrous Na₂SO₄, filtered, and concentrated to dryness under reduced pressure to afford the title product, which was purified by silica gel chromatography (eluent: petroleum ether:ethyl acetate=1:0 to 0:1) to afford a brown oil (190 mg, 95% purity, 53%). LCMS (ESI): mass calcd. for C₂₈H₂₅N₅O₄S₂ 559.66 m/z, found 560.0 [M+H]⁺.

### Intermediate 131. 3-(3-bromo-5-methylphenyl)-3-hydroxy-1-methylpyrrolidin-2-one

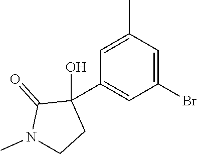 [SMILES: Cc1cc(Br)cc(C2(O)CCN(C)C2=O)c1]

Step A. 3-(3-Bromo-5-methylphenyl)-3-hydroxy-1-methylpyrrolidin-2-one. n-BuLi (1.4 mL, 2.5 M in hexane, 3.5 mmol) was added drop-wise to a −70° C. (dry ice/EtOH) THF solution of 1,3-dibromo-5-methylbenzene (862 mg, 3.45 mmol) in THF (15 mL). The resultant mixture was stirred at −70° C. for 30 min before treating with a THF solution of 1-methylpyrrolidine-2,3-dione (300 mg, 2.65 mmol). The mixture was stirred at −50° C. for 30 min and then quenched with sat. NH₄Cl (20 mL) and extracted with ethyl acetate (10 mL×3). The combined organic extracts were dried over anhydrous Na₂SO₄, filtered, and concentrated under reduced pressure to afford the product, which was purified by silica gel chromatography (petroleum ether:ethyl acetate=10:1 to 0:1) to afford the title compound (420 mg, 52%) as a light yellow solid. ¹H NMR (400 MHz, DMSO-d₆) δ 7.32 (d, J=19.3 Hz, 2H), 7.12 (s, 1H), 6.10 (s, 1H), 3.46-3.38 (m, 1H), 3.33-3.28 (m, 1H), 2.82 (s, 3H), 2.34-2.25 (m, 4H), 2.23-2.14 (m, 1H).

Step B. (S)-3-(3-Bromo-5-methylphenyl)-3-hydroxy-1-methylpyrrolidin-2-one. 3-(3-Bromo-5-methylphenyl)-3-hydroxy-1-methylpyrrolidin-2-one (420 mg, 1.48 mmol) was further purified by SFC over DAICEL CHIRALPAK AS-H 250 mm×30 mm, 5 μm (eluent: 35% to 35% (v/v) supercritical CO₂ in EtOH and H₂O with 0.1% NH₃) to give two enantiomers. The first eluting enantiomer was obtained (200 mg, 42%) obtained as a yellow solid. ¹H NMR (400 MHz, DMSO-d₆) δ 7.32 (d, J=18.8 Hz, 2H), 7.12 (s, 1H), 6.10 (s, 1H), 3.45-3.38 (m, 1H), 3.33-3.30 (m, 1H), 2.82 (s, 3H), 2.34-2.25 (m, 4H), 2.23-2.15 (m, 1H).

Step C. (R)-3-(3-Bromo-5-methylphenyl)-3-hydroxy-1-methylpyrrolidin-2-one. The second eluting enantiomer was (200 mg, 44%) obtained as a yellow solid. ¹H NMR (400 MHz, DMSO-d₆) δ 7.32 (d, J=19.3 Hz, 2H), 7.12 (s, 1H), 6.10 (s, 1H), 3.45-3.39 (m, 1H), 3.33-3.29 (m, 1H), 2.82 (s, 3H), 2.34-2.25 (m, 4H), 2.23-2.14 (m, 1H).

### Intermediate 132. (R)-3-hydroxy-1-methyl-3-(3-methyl-5-(4,4,5,5-tetramethyl-1,3,2-dioxaborolan-2-yl)phenyl)pyrrolidin-2-one

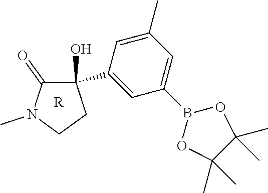

To a microwave vial containing (R)-3-(3-Bromo-5-methylphenyl)-3-hydroxy-1-methylpyrrolidin-2-one (180 mg, 0.633 mmol), 4,4,4′,4′,5,5,5′,5′-octamethyl-2,2′-bi(1,3,2-dioxaborolane) (322 mg, 1.27 mmol), and KOAc (187 mg, 1.91 mmol), was added 1,4-dioxane (8 mL). The mixture was sparged with N₂ for 5 minutes and then treated with Pd(dppf)Cl₂ (46 mg, 0.063 mmol). The resultant mixture was heated to 70° C. via microwave irradiation for 1 hour. The reaction vessel was removed from the microwave and allowed to gradually cool to room temperature. The reaction mixture was then filtered through a pad of diatomaceous earth. The filtrate was concentrated to dryness under reduced pressure to give the product, which was purified by silica gel chromatography (eluent: petroleum ether ethyl acetate=1:0 to 0:1) to afford the title compound (170 mg) as a brown oil. LCMS (ESI): mass calcd. for C₁₈H₂₆BNO₄ 331.21 m/z, found 332.2 [M+H]⁺.

### Intermediate 133. (R)-3-hydroxy-1-methyl-3-(3-methyl-5-(2-(5-tosyl-5H-pyrrolo[2,3-b]pyrazin-7-yl)thiazol-4-yl)phenyl)pyrrolidin-2-one

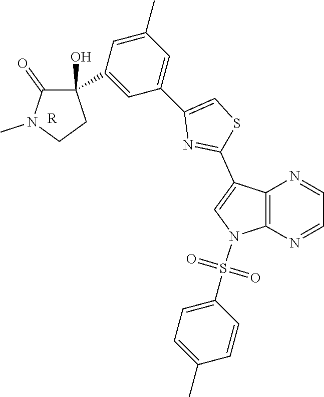

To a microwave vial containing (R)-3-Hydroxy-1-methyl-3-(3-methyl-5-(4,4,5,5-tetramethyl-1,3,2-dioxaborolan-2-yl)phenyl)pyrrolidin-2-one (Intermediate 132, 170 mg, 0.513 mmol), 4-bromo-2-(5-tosyl-5N-pyrrolo[2,3-b]pyrazin-7-yl)thiazole (Intermediate 10, 246 mg, 0.565 mmol), and K₃PO₄ (327 mg, 1.54 mmol), was added 1,4-dioxane (14 mL), and H₂O (3 mL). The mixture was sparged with Ar for 5 minutes and then treated with Pd(dtbpf)Cl₂ (34 mg, 0.052 mmol). The mixture was sparged again with Ar for another 5 minutes and then heated at 80° C. via microwave irradiation for 1 hour. The reaction mixture cooled to room temp and filtered through a pad of diatomaceous earth and the pad washed with ethyl acetate (30 mL). The filtrate was poured into H₂O (20 mL) and extracted ethyl acetate (20 mL×3). The combined organic extracts were washed with brine, dried over anhydrous Na₂SO₄, filtered, and concentrated under reduced pressure to afford the product, which was purified by silica gel chromatography (eluent: petroleum ether:ethyl acetate=1:0 to 0:1) to afford the title compound (160 mg, 52%) as a brown oil. LCMS (ESI): mass calcd. for C₂₈H₂₅N₅O₄S₂ 559.66 m/z, found 560.10 [M+H]⁺.

### Intermediate 134. (S)-3-hydroxy-1-methyl-3-(2-methyl-5-(4,4,5,5-tetramethyl-1,3,2-dioxaborolan-2-yl)phenyl)pyrrolidin-2-one

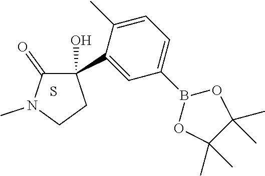

To a microwave vial containing (S)-3-(5-Bromo-2-methylphenyl)-3-hydroxy-1-methylpyrrolidin-2-one (Intermediate 128, 70 mg, 0.25 mmol), 4,4,4′,4′,5,5,5′,5′-octamethyl-2,2′-bi(1,3,2-dioxaborolane) (100 mg, 0.394 mmol) and KOAc (73 mg, 0.74 mmol) was added 1,4-dioxane (5 mL). The mixture was sparged with N₂ for 5 minutes and then treated with Pd(dppf)Cl₂ (18 mg, 0.03 mmol). The resultant mixture was heated at 85° C. via microwave irradiation for 1 hour. The reaction mixture was cooled to room temperature and the suspension was filtered through a pad of diatomaceous earth and the pad was washed with ethyl acetate (30 mL). The filtrate was concentrated under reduced pressure to afford the product, which was purified by silica gel chromatography (eluent: petroleum ether:ethyl acetate=10:1 to 1:1) to afford the title compound (70 mg) as a brown oil. LCMS (ESI): mass calcd. for C₁₈H₂₈BNO₄ 331.20 m/z, found 332.2 [M+H]⁺.

### Intermediate 135. (R)-3-hydroxy-1-methyl-3-(2-methyl-5-(4,4,5,5-tetramethyl-1,3,2-dioxaborolan-2-yl)phenyl)pyrrolidin-2-one

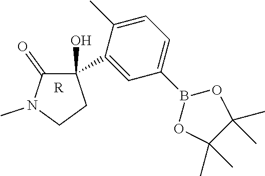

(R)-3-(5-Bromo-2-methylphenyl)-3-hydroxy-1-methylpyrrolidin-2-one (Intermediate 127, 110 mg, 0.387 mmol) was added to a mixture of 4,4,4′,4′,5,5,5′,5′-octamethyl-2,2′-bi(1,3,2-dioxaborolane) (157 mg, 0.618 mmol), KOAc (103 mg, 1.05 mmol), and 1,4-dioxane (6 mL). The mixture was sparged with Ar for 5 minutes and then treated with Pd(dppf)Cl₂ (30 mg, 0.041 mmol). The mixture was sparged with Ar for another 5 minutes and then heated to 80° C. via microwave irradiation for 1 hour. The suspension concentrated under reduced pressure to afford the product, which was purified by silica gel chromatography (eluent: petroleum ether:ethyl acetate=1:0 to 0:1) to afford the title compound (110 mg, 56%) as a yellow oil. LC-MS (ESI): mass calcd. For C₁₈H₂₆BNO₄ 331.20 m/z found 332.2[M+H]⁺.

### Intermediate 136. (R)-3-hydroxy-1-methyl-3-(3-(2-(2-methyl-1-tosyl-1H-pyrrolo[2,3-b]pyridin-3-yl)thiazol-4-yl)phenyl)pyrrolidin-2-one

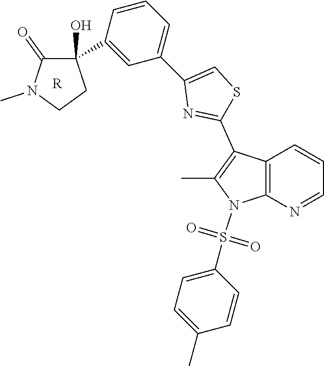

Step A. 3-Bromo-2-methyl-1-tosyl-1H-pyrrolo[2,3-b]pyridine. To a flask containing TosCl (650 mg, 3.41 mmol), 3-bromo-2-methyl-1H-pyrrolo[2,3-b]pyridine (600 mg, 2.84 mmol), DMAP (70 mg, 0.57 mmol), and Et₃N (2.0 ml, 14 mmol) was added dichloromethane (8 mL). The resultant mixture was stirred at room temperature for 12 hours. The mixture was concentrated to dryness under reduced pressure to afford the product, which was purified by silica gel chromatography (0-33% EtOAc/pet ether) to give the title compound (840 mg, 80.5%) as a yellow solid. LC-MS (ESI): mass calcd. For C₁₅H₁₃BrN₂O₂S 365.24 m/z found 366.9 [M+H]⁺.

Step B. 2-Methyl-3-(4,4,5,5-tetramethyl-1,3,2-dioxaborolan-2-yl)-1-tosyl-1H-pyrrolo[2,3-b]pyridine. Pd(dppf)Cl₂ (80 mg, 0.11 mmol) was added to a solution of 3 bromo-2-methyl-1-tosyl-1H-pyrrolo[2,3-b]pyridine (400 mg, 1.10 mmol), 4,4,4′,4′,5,5,5′,5′-octamethyl-2,2′-bi(1,3,2-dioxaborolane) (556 mg, 2.19 mmol), KOAc (322 mg, 3.28 mmol), and 1,4-dioxane (5 mL). The resultant mixture was heated at 85° C. for 12 hours under N₂. The reaction vessel was removed from the oil bath and allowed to gradually cool to room temperature The reaction mixture was then filtered through a pad of diatomaceous earth and the pad was washed with ethyl acetate (70 mL). The filtrate was concentrated to dryness under reduced pressure to afford the product, which was purified by silica gel chromatography (0-33% EtOAc/pet ether) to give the title compound (300 mg) as a white solid. LC-MS (ESI): mass calcd. For C₂₁H₂₅BN₂O₄S 412.31 m/z found 413.2 [M+H]⁺.

Step C. 4-Bromo-2-(2-methyl-1-tosyl-1H-pyrrolo[2,3-b]pyridin-3-yl)thiazole. 2-Methyl-3-(4,4,5,5-tetramethyl-1,3,2-dioxaborolan-2-yl)-1-tosyl-1H-pyrrolo[2,3-b]pyridine (300 mg, 0.728 mmol) was added to a solution of 2,4-dibromothiazole (265 mg, 1.09 mmol), Cs₂CO₃ (592 mg, 1.82 mmol), 1,4-dioxane (15 mL), and H₂O (3 mL). The mixture was sparged with Ar for 5 minutes and then treated with Pd(dppf)Cl₂ (53 mg, 0.072 mmol). The mixture was sparged with Ar for another 5 minutes and then stirred at 85° C. for 3 hour. The reaction vessel was removed from oil bath and allowed to gradually cool to room temperature The reaction mixture was then filtered through a pad of diatomaceous earth and the pad was washed with ethyl acetate (50 mL). The filtrate was concentrated to dryness under reduced pressure to afford the product, which was subjected to silica gel chromatography (0-33% EtOAc/pet ether) to afford the title compound (230 mg, 52%) as a brown solid. LC-MS (ESI): mass calcd. For C₁₈H₁₄BrN₃O₂S₂ 448.35 m/z found 449.9 [M+H]⁺.

Step D. (R)-3-Hydroxy-1-methyl-3-(3-(2-(2-methyl-1-tosyl-1H-pyrrolo[2,3-b]pyridin-3-yl)thiazol-4-yl)phenyl)pyrrolidin-2-one. To a microwave vial containing 4-(8-bromoisoquinolin-3-yl)-1H-pyrazol-3-amine (100 mg, 0.223 mmol), (R)-3-hydroxy-1-methyl-3-(3-(4,4,5,5-tetramethyl-1,3,2-dioxaborolan-2-yl)phenyl)pyrrolidin-2-one (Intermediate 16, 60 mg, 0.189 mmol), and K₃PO₄ (91 mg, 0.67 mmol) was added 1,4 dioxane (8 mL), and H₂O (2 mL). The mixture was sparged with Ar for 5 minutes and then treated with Pd(dtbpf)Cl₂ (15 mg, 0.023 mmol). The mixture was sparged with Ar for another 5 minutes and then heated at 100° C. via microwave irradiation for 1 hour. The reaction vessel was gradually cooled to room temperature, filtered through a pad of diatomaceous earth and the pad was washed with ethyl acetate (20 mL). The filtrate was diluted with water H₂O (20 mL) and extracted with ethyl acetate (30 mL×3). The combined organic extracts were washed with brine, dried over anhydrous Na₂SO₄, filtered, and concentrated to dryness under reduced pressure to afford the product, which was purified by silica gel chromatography (eluent: petroleum ether:ethyl acetate=1:0 to 0:1) to afford the title compound (120 mg, 84%) as a white solid. LC-MS (ESI): mass calcd. For C₂₉H₂₆N₄O₄S₂ 558.67 m/z found 559.2 [M+H]⁺.

### Intermediate 137. (R)-3-(4-(3-(3-hydroxy-1-methyl-2-oxopyrrolidin-3-yl)phenyl)thiazol-2-yl)-1-tosyl-1H-pyrrolo[2,3-b]pyridine-5-carbonitrile

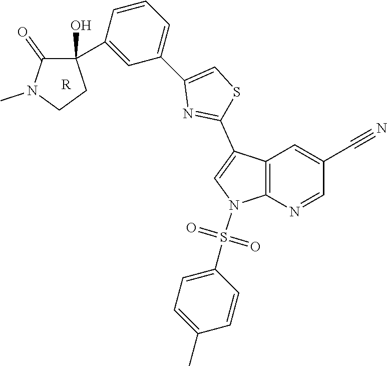

Step A. 3-Bromo-1-tosyl-1H-pyrrolo[2,3-b]pyridine-5-carbonitrile. TosCl (515 mg, 2.70 mmol) was added to a solution of 3-bromo-1H-pyrrolo[2,3-b]pyridine-5-carbonitrile (500 mg, 2.25 mmol), DMAP (55 mg, 0.45 mmol), Et₃N (1.6 ml, 12 mmol), and dichloromethane (10 mL) in a 100 mL three-necked round bottom flask. The resultant mixture was stirred at room temperature for 15 hours. After this time, the mixture was concentrated to dryness under reduced pressure to afford the product, which was purified by silica gel chromatography (eluent: petroleum ether ethyl acetate=1:0 to 5:1) to afford the title compound (900 mg, 98%) as a white solid. LC-MS (ESI): mass calcd. For C₁₅H₁₀BrN₃O₂S 376.22 m/z found 377.9 [M+H]⁺.

Step B. 3-(4,4,5,5-Tetramethyl-1,3,2-dioxaborolan-2-yl)-1-tosyl-1H-pyrrolo[2,3-b]pyridine-5-carbonitrile. To a flask containing 3-bromo-1-tosyl-1H-pyrrolo[2,3-b]pyridine-5-carbonitrile (880 mg, 2.34 mmol), 4,4,4′,4′,5,5,5′,5′-octamethyl-2,2′-bi(1,3,2-dioxaborolane) (1.19 g, 4.69 mmol) and KOAc (689 mg, 7.02 mmol) was added 1,4-dioxane (10 mL). The mixture was sparged with N₂ for 5 minutes and then treated with Pd(dppf)Cl₂ (171 mg, 0.234 mmol). The resultant mixture was heated at 85° C. for 16 hours and then cooled to room temperature. The suspension was filtered through a pad of diatomaceous earth and the eluent was concentrated to dryness under reduced pressure to give the product, which was purified by silica gel chromatography (eluent: petroleum ether:ethyl acetate=1:0 to 5:1) to afford the title compound (550 mg) as a white solid. LC-MS (ESI): mass calcd. For C₂₁H₂₂BN₃O₄S 423.2 m/z found 341.9 [M−82+H]⁺.

Step C. 3-(4-Bromothiazol-2-yl)-1-tosyl-1H-pyrrolo[2,3-b]pyridine-5-carbonitrile. 3-(4,4,5,5-tetramethyl-1,3,2-dioxaborolan-2-yl)-1-tosyl-1H-pyrrolo[2,3-b]pyridine-5-carbonitrile (530 mg, 1.25 mmol) was added to a solution of 2,4-dibromothiazole (456 mg, 1.88 mmol), Cs₂CO₃ (1.02 g, 3.13 mmol), 1,4-dioxane (5 mL), and H₂O (1 mL). The mixture was sparged with Ar for 5 minutes and then treated with Pd(dppf)Cl₂ (92 mg, 0.13 mmol). The mixture was sparged with Ar for another 5 minutes and then heated at 85° C. via microwave irradiation for 1 hour. The reaction mixture was cooled to room temperature and the suspension was filtered through a pad of diatomaceous earth which was washed with ethyl acetate (50 mL). The mixture was concentrated under reduced pressure to afford the product, which was purified by silica gel chromatography (eluent: petroleum ether:ethyl acetate=0:1 to 3:1). The product was further purified by preparative HPLC using a Xtimate C18 150×40 mm, 10 μm (eluent: 25% to 55% (v/v) CH₃CN and water (0.2% FA)) to afford pure product. The product was suspended in water (5 mL), the mixture frozen using dry ice, and then lyophilized to dryness to afford the title compound (130 mg) as a yellow solid. ¹H NMR (400 MHz, CDCl₃) δ 8.96 (d, J=1.8 Hz, 1H), 8.74 (d, J=1.5 Hz, 1H), 8.38 (s, 1H), 8.14 (d, J=8.4 Hz, 2H), 7.35 (d, J=8.4 Hz, 2H), 7.26 (s, 1H), 2.42 (s, 3H).

Step D. (R)-3-(4-(3-(3-Hydroxy-1-methyl-2-oxopyrrolidin-3-yl)phenyl)thiazol-2-yl)-1-tosyl-1H-pyrrolo[2,3-b]pyridine-5-carbonitrile. To a microwave vial containing 3-(4-Bromothiazol-2-yl)-1-tosyl-1H-pyrrolo[2,3-b]pyridine-5-carbonitrile (100 mg, 0.218 mmol), (R)-3-hydroxy-1-methyl-3-(3-(4,4, 5,5-tetramethyl-1,3,2-dioxaborolan-2-yl)phenyl)pyrrolidin-2-one (Intermediate 16, 58 mg, 0.18 mmol) and K₃PO₄ (116 mg, 0.546 mmol) was added 1,4-dioxane (4 mL), and H₂O (0.8 mL). The mixture was sparged with Ar for 5 minutes and then treated with Pd(dtbpf)Cl₂ (12 mg, 0.018 mmol). The mixture was sparged with Ar for another 5 minutes and then heated at 85° C. for 1 hour. The reaction mixture was cooled to room temperature and the solvent was removed under reduced pressure to afford product, which was purified by silica gel chromatography (eluent: petroleum ether:ethyl acetate (containing 10% CH₃OH)=1:0 to 0:1) to afford the title compound (90 mg), as a white solid. LC-MS (ESI): mass calcd. For C₂₉H₂₃N₅O₄S₂ 569.6 m/z found 570.2 [M+H]⁺.

### Intermediate 138. (R)-3-hydroxy-1-methyl-3-(3-(2-(6-methyl-1-tosyl-1H-pyrrolo[2,3-b]pyridin-3-yl)thiazol-4-yl)phenyl)pyrrolidin-2-one

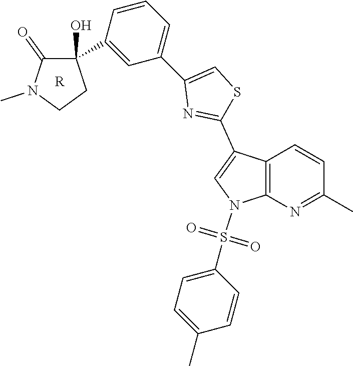

Step A. 3-Bromo-6-methyl-1-tosyl-1H-pyrrolo[2,3-b]pyridine. TosCl (542 mg, 2.84 mmol) was added to a solution of 3-bromo-6-methyl-1H-pyrrolo[2,3-b]pyridine (500 mg, 2.37 mmol), DMAP (58 mg, 0.48 mmol), Et₃N (1.65 mL, 11.9 mmol), and dichloromethane (10 mL) in a 100 mL three-necked round bottom flask. The resultant mixture was stirred at room temperature for 15 hours. The filtrate was concentrated to dryness under reduced pressure to afford the product, which was purified by silica gel chromatography (petroleum ether:ethylacetate=1:0 to 5:1) to afforded the title compound (892 mg, 99%) as a white solid. LC-MS (ESI): mass calcd. For C₁₅H₁₃BrN₂O₂S 365.2 m/z found 366.9 [M+H]⁺.

Step B. 6-Methyl-3-(4,4,5,5-tetramethyl-1,3,2-dioxaborolan-2-yl)-1-tosyl-1H-pyrrolo[2,3-b]pyridine. To a flask containing 3-Bromo-6-methyl-1-tosyl-1H-pyrrolo[2,3-b]pyridine (800 mg, 2.19 mmol), 4,4,4′,4′,5,5,5′,5′-octamethyl-2,2′-bi(1,3,2-dioxaborolane) (1.11 g, 4.37 mmol) and KOAc (645 mg, 6.57 mmol) was added 1,4-dioxane (10 mL). The mixture was sparged with N₂ for 5 minutes and then treated with Pd(dppf)Cl₂ (160 mg, 0.22 mmol). The resultant mixture was heated at 85° C. for 16 hours. The mixture was cooled to room temperature and the suspension was filtered through a pad of diatomaceous earth and the eluent concentrated to dryness under reduced pressure to give the product, which was purified by silica gel chromatography (eluent: petroleum ether:ethyl acetate=1:0 to 5:1) to afford the title compound (520 mg) as a white solid. LC-MS (ESI): mass calcd. For C₂₁H₂₅BN₂O₄S 412.31 m/z found 413.1 [M+H]⁺.

Step C. 4-Bromo-2-(6-methyl-1-tosyl-1H-pyrrolo[2,3-b]pyridin-3-yl)thiazole. To a flask containing of 2,4-dibromothiazole (442 mg, 1.82 mmol), Cs₂CO₃ (988 mg, 3.03 mmol), 1,4-dioxane (5 mL), and H₂O (1 mL) was added 6-Methyl-3-(4,4,5,5-tetramethyl-1,3,2-dioxaborolan-2-yl)-1-tosyl-1H-pyrrolo[2,3-b]pyridine (500 mg, 1.21 mmol). The mixture was sparged with Ar for 5 minutes and then treated with Pd(dppf)Cl₂ (89 mg, 0.12 mmol). The mixture was sparged with Ar for another 5 minutes and heated at 85° C. for 3 hours. The reaction mixture was cooled to room temperature and the suspension was filtered through a pad of diatomaceous earth which was washed with ethyl acetate (50 mL). The mixture was concentrated to dryness under reduced pressure to afford the product, which was purified by silica gel chromatography (eluent: petroleum ether:ethyl acetate=0:1 to 3:1) to afford the title compound (200 mg, 36%) as a brown solid. LC-MS (ESI): mass calcd. For C₁₈H₁₄BrN₃O₂S₂ 448.3 m/z found 449.8 [M+H]⁺. ¹H NMR (400 MHz, MeOD-d₄) δ 8.53-8.31 (m, 2H), 8.13 (d, J=7.6 Hz, 2H), 7.54 (s, 1H), 7.39 (d, J=6.8 Hz, 2H), 7.27 (d, J=8.1 Hz, 1H), 2.64 (s, 3H), 2.39 (s, 3H).

Step D. (R)-3-Hydroxy-1-methyl-3-(3-(2-(6-methyl-1-tosyl-1H-pyrrolo[2,3-b]pyridin-3-yl)thiazol-4-yl)phenyl)pyrrolidin-2-one. To a microwave vial containing 4-bromo-2-(6-methyl-1-tosyl-1H-pyrrolo[2,3-b]pyridin-3-yl)thiazole (100 mg, 0.223 mmol), (R)-3-hydroxy-1-methyl-3-(3-(4,4, 5,5-tetramethyl-1,3,2-dioxaborolan-2-yl)phenyl)pyrrolidin-2-one (Intermediate 16, 59 mg, 0.186 mmol) and K₃PO₄ (118 mg, 0.556 mmol) was added 1,4-dioxane (4 mL), and H₂O (0.8 mL). The mixture was sparged with Ar for 5 minutes and then treated with Pd(dtbpf)Cl₂ (12 mg, 0.018 mmol). The mixture was sparged with Ar for another 5 minutes and then heated at 85° C. for 1 hour. The reaction mixture was cooled to room temperature and then filtered through a pad of diatomaceous earth which was washed with ethyl acetate (30 mL). The solvent was removed under reduced pressure to afford the product, which was purified by silica gel chromatography (eluent: petroleum ether:ethyl acetate (containing 10% CH₃OH)=1:0 to 0:1) to afford the compound (120 mg, 99%) as a white solid. LC-MS (ESI): mass calcd. For C₂₉H₂₆N₄O₄S₂ 558.6 m/z found 559.1 [M+H]⁺.

### Intermediate 139. 1-(1-Tosyl-1H-imidazol-2-yl)-1-(3-(2-(5-tosyl-5H-pyrrolo[2,3-b]pyrazin-7-yl)thiazol-4-yl)phenyl)ethan-1-ol

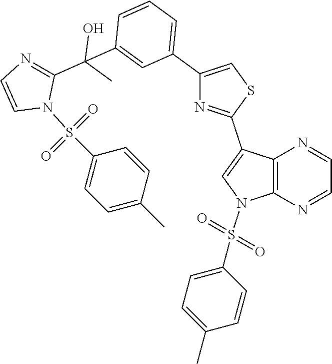 [SMILES: Cc1ccc(S(=O)(=O)n2ccnc2C(C)(O)c2cccc(-c3csc(-c4cn(S(=O)(=O)c5ccc(C)cc5)c5nccnc45)n3)c2)cc1]

Step A. 1-(1-Tosyl-1H-imidazol-2-yl)ethenone. 4-Toluenesulfonyl chloride (8.3 g, 44 mmol) was added to a 250 mL round-bottomed flask containing of 1-(1H-imidazol-2-yl)ethanone (4.0 g, 36 mmol), Et₃N (7.6 ml, 55 mmol), and methylene chloride (100 mL). The resultant mixture was stirred at room temp for 12 hours. After this time, the mixture was concentrated under reduced pressure to afford the product, which was purified by silica gel chromatography (eluent: petroleum ether:ethyl acetate=1:0 to 0:1) to afford the title compound (7.1 g, 73%) as a yellow solid. LC-MS (ESI): mass calcd. For C₁₂H₁₂N₂O₃S 264.06 m/z, found 264.9 [M+H]⁺. 1H NMR (400 MHz, CDCl₃) δ 7.99 (d, J=8.6 Hz, 2H), 7.87 (d, J=1.5 Hz, 1H), 7.35 (d, J=8.1 Hz, 2H), 7.17 (d, J=1.5 Hz, 1H), 2.56 (s, 3H), 2.43 (s, 3H).

Step B. 1-(3-Bromophenyl)-1-(1-tosyl-1H-imidazol-2-yl)ethanol. n-Butyllithium (9.5 mL, 2.0 M in hexane, 19 mmol) was added dropwise to a −70° C. (dry ice/EtOH) solution of 1,3-dibromobenzene (4.50 g, 19.1 mmol) and anhydrous THF (45 mL) under N₂ atmosphere. The resultant mixture was stirred at −70° C. for 1 h and then treated with a solution of 1-(1-tosyl-1H-imidazol-2-yl)ethanone (2.5 g, 9.5 mmol) and THF (15 mL), and stirred at −70° C. for 3 hours. After this time, the mixture was quenched with sat. NH₄Cl (100 mL), warmed to room temperature, and extracted with ethyl acetate (200 mL×3). The combined organic extracts were dried over anhydrous Na₂SO₄, filtered, and concentrated under reduced pressure to afford the product, which was purified by silica gel chromatography (eluent: petroleum ether:ethyl acetate=1:0 to 1:1) to give the title compound (2.4 g, 39%) as a yellow solid. LC-MS (ESI): mass calcd. For C₁₈H₁₇BrN₂O₃S 420.01 m/z, found 421.1 [M+H]⁺.

Step C. 1-(3-(4,4,5,5-Tetramethyl-1,3,2-dioxaborolan-2-yl)phenyl)-1-(1-tosyl-1H-imidazol-2-yl)ethanol. To a microwave vial containing 1-(3-Bromophenyl)-1-(1-tosyl-1H imidazol-2-yl)ethanol (500 mg), 4,4,4′,4′,5,5,5′,5′-octamethyl-2,2′-bi(1,3,2-dioxaborolane) (368 mg, 1.45 mmol) and KOAc (213 mg, 2.17 mmol) was added 1,4-dioxane (10 mL). The mixture was sparged with Ar for 5 minutes and then treated with Pd(dppf)Cl₂ (53 mg, 0.072 mmol). The mixture was sparged with Ar for another 5 minutes and then heated at 90° C. via microwave irradiation for 1 hour. After this time, the mixture was cooled to room temperature and combined with another reaction mixture in which 500 mg (0.724 mmol) of 1-(3-bromophenyl)-1-(1-tosyl-1H-imidazol-2-yl)ethanol was used. The combined mixture was filtered through a pad of diatomaceous earth and the pad was washed with ethyl acetate (50 mL). The filtrate was washed with water (50 mL), dried over anhydrous Na₂SO₄, filtered, and concentrated under reduced pressure to afford the product, which was purified by silica gel chromatography (eluent: petroleum ether:ethyl acetate=1:0 to 3:1) to give the title compound (700 mg) as a brown solid. LC-MS (ESI): mass calcd. For C₂₄H₂₉BN₂O₅S 468.19 m/z found 469.6 [M+H]⁺.

Step D. 1-(1-Tosyl-1H-imidazol-2-yl)-1-(3-(2-(5-tosyl-5H-pyrrolo[2,3-b]pyrazin-7-yl)thiazol-4-yl)phenyl)ethanol. To a microwave vial containing 1-(3-(4,4,5,5-Tetramethyl-1,3,2-dioxaborolan-2-yl)phenyl)-1-(1-tosyl-1H-imidazol-2-yl)ethanol (500 mg), 4-bromo-2-(5-tosyl-5H-pyrrolo[2,3-b]pyrazin-7-yl)thiazole (Intermediate 10, 300 mg, 0.689 mmol) and K₃PO₄ (476 mg, 2.24 mmol) was added 1,4-dioxane (10 mL), and H₂O (2.5 mL). The resultant mixture was sparged with Ar for 5 minutes and then treated with Pd(dtbpf)Cl₂ (49 mg, 0.075 mmol). The mixture was sparged with Ar for another 5 minutes and then heated at 100° C. via microwave irradiation for 2 hours. After this time, the mixture was cooled to room temperature, filtered through a pad of diatomaceous earth, and the pad was washed with ethyl acetate (20 mL). The filtrate was poured into water (20 mL) and extracted with ethyl acetate (30 mL×3). The combined organic extracts were washed with brine, dried over anhydrous Na₂SO₄, filtered, and concentrated under reduced pressure to give the product, which was purified by silica gel chromatography (eluent: petroleum ether:ethyl acetate=1:0 to 1:1) to afford the title compound (80 mg, 14%) as a yellow solid. LC-MS (ESI): mass calcd. For C₃₄H₂₈N₈O₅S₃ 696.13 m/z found 697.1 [M+H]⁺.

### Intermediate 140. 1-(1-Methyl-1H-imidazol-2-yl)-1-(3-(2-(1-(phenylsulfonyl)-1H-pyrrolo[2,3-b]pyridin-3-yl)thiazol-4-yl)phenyl)ethan-1-ol

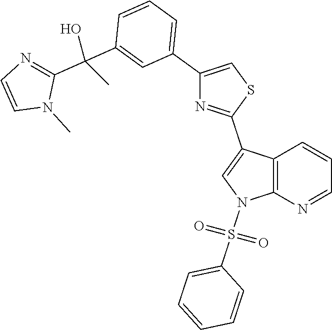 [SMILES: Cn1ccnc1C(C)(O)c1cccc(-c2csc(-c3cn(S(=O)(=O)c4ccccc4)c4ncccc34)n2)c1]

Step A. 1-(3-(2-(1-(Phenylsulfonyl)-1H-pyrrolo[2,3-b]pyridin-3-yl)thiazol-4-yl)phenyl)-1-(1-tosyl-1H-imidazol-2-yl)ethanol. n-Butyllithium (20 mL, 50 mmol, 2.5 M in hexane) was added dropwise to a −72° C. (dry ice/ethanol) solution of 1,3-dibromobenzene (6.0 mL, 50 mmol) and anhydrous THF (60 mL). The resultant mixture was stirred at −72° C. for 1 hour, and then treated with a solution of 1-(1-methyl-1H imidazol-2-yl)ethanone (4.80 g, 38.7 mmol) in anhydrous THF (6 mL) and stirred at −72° C. for another 1 hour. After this time, the reaction mixture was quenched with saturated NH₄Cl (50 mL), diluted with water (50 mL) and extracted with ethyl acetate (30 mL×6). The combined organic extracts were washed with brine, dried over anhydrous Na₂SO₄, filtrated, and concentrated under reduced pressure to give the product, which was purified by silica gel chromatography (eluent: CH₂Cl₂:MeOH=1:0 to 10:1) to afford the title compound (5.3 g, 93.5% purity, 45%) as a white solid. LC-MS (ESI): mass calcd. For C₁₂H₁₃BrN₂O 280.02 m/z, found 280.9 [M+H]⁺.

Step B. 1-(1-Methyl-1H-imidazol-2-yl)-1-(3-(4,4,5,5-tetramethyl-1,3,2-dioxaborolan-2-yl)phenyl)ethanol. [1,1′-Bis(diphenylphosphino)ferrocene]-dichloropalladium(II) (1.2 g, 1.6 mmol) and KOAc (5.0 g, 51 mmol) were added to a solution of 1-(3-bromophenyl)-1-(1-methyl-1H-imidazol-2-yl)ethanol (5.0 g, 17 mmol, 93.5% purity), 4,4,4′,4′,5,5,5′,5′-octamethyl-2,2′-bi(1,3,2-dioxaborolane) (8.5 g, 33 mmol), and 1,4-dioxane (80 mL). The mixture was sparged with Ar for 5 minutes and then heated at 90° C. for 16 hours. After this time, the mixture was cooled to room temperature, filtered through a pad of diatomaceous earth, and the pad was washed with ethyl acetate (50 mL). The filtrate was diluted with water (50 mL) and extracted with ethyl acetate (3×50 mL). The combined organic extracts were dried over anhydrous Na₂SO₄, filtered, and concentrated under reduced pressure to afford the product, which was further purified by silica gel chromatography (eluent: CH₂Cl₂:MeOH=1:0 to 10:1) to give the title compound (2.2 g, 36%) as a brown solid. LC-MS (ESI): mass calcd. For C₁₈H₂₅BN₂O₃ 328.20 m/z, found 329.1 [M+H]⁺.

Step C. 1-(1-Methyl-1H-imidazol-2-yl)-1-(3-(2-(1-(phenylsulfonyl)-1H-pyrrolo[2,3-b]pyridin-3-yl)thiazol-4-yl)phenyl)ethanol. To a microwave vial containing 1-(1-Methyl-1H-imidazol-2-yl)-1-(3-(4,4,5,5-tetramethyl-1,3,2-dioxaborolan-2-yl)phenyl)ethanol (600 mg, 1.64 mmol, 90% purity), 4-bromo-2-(1-(phenylsulfonyl)-1H-pyrrolo[2,3-b]pyridin-3-yl)thiazole (Intermediate 2, 0.90 g, 1.1 mmol, 53% purity) was added 1,4-dioxane/H₂O (5 mL, v:v=4:1). The mixture was sparged with Ar for 5 minutes, treated with Pd(dtbpf)Cl₂ (120 mg, 0.184 mmol) and K₃PO₄ (1.1 g, 5.2 mmol), and sparged with Ar for another 5 minutes. The resultant mixture was heated at 85° C. via microwave irradiation for 1.5 hours. After this time, the mixture was cooled to room temperature, filtered through a pad of diatomaceous earth and the pad washed with ethyl acetate (30 mL). The filtrate was diluted with water (20 mL) and extracted with ethyl acetate (3×20 mL). The organic extracts were dried over anhydrous Na₂SO₄, filtered, and concentrated under reduced pressure to afford the product, which was purified by silica gel chromatography using a spherical C18 column (eluent: MeCN:H₂O=5:95 to 70:30) to give the title compound (420 mg, 43%) as a colorless solid. LC-MS (ESI): mass calcd. For C₂₈H₂₃N₅O₃S₂ 541.12 m/z, found 542.2 [M+H]⁺.

### Intermediate 141. (R)-7-(3-(2-(2-methyl-5-tosyl-5H-pyrrolo[2,3-b]pyrazin-7-yl)thiazol-4-yl)phenyl)-6,7-dihydro-5H-pyrrolo[1,2-a]imidazol-7-ol

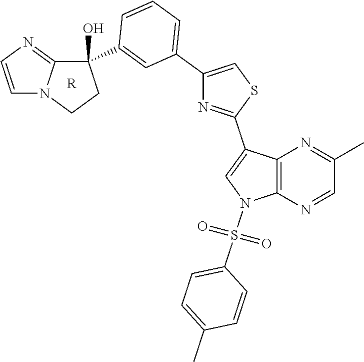

Step A. 2-Methyl-5-tosyl-5H-pyrrolo[2,3-b]pyrazine. To a flask containing 2-Bromo-5-tosyl-5H-pyrrolo[2,3-b]pyrazine (3.5 g, 9.9 mmol), trimethylboroxine (3.74 g, 29.8 mmol) and K₃PO₄ (3.43 g, 24.8 mmol) was added 1,4-dioxane (60 mL). The mixture was sparged with Ar for 5 minutes and then treated with Pd(dppf)Cl₂ (727 mg, 0.994 mmol). The mixture was sparged with Ar for another 5 minutes and the resultant mixture was heated at 115° C. for 3 hours. After this time, the mixture was cooled to room temperature, filtered through a pad of diatomaceous earth and the pad was washed with ethyl acetate (100 mL). The filtrate was concentrated under reduced pressure to give the product, which was purified by silica gel chromatography (eluent: petroleum ether:ethyl acetate=20:1 to 5:1) to afford the title compound (2.5 g, 88%) as a white solid. LCMS (ESI): mass calcd. for C₁₄H₁₃N₃O₂S 287.07 m/z, found 288.1 [M+H]⁺.

Step B. 7-Bromo-2-methyl-5-tosyl-5H-pyrrolo[2,3-b]pyrazine. NBS (1.2 g, 6.7 mmol) was added to a solution of 2-methyl-5-tosyl-5H-pyrrolo[2,3-b]pyrazine (1.6 g, 5.6 mmol) and DMF (20 mL). The resultant mixture was stirred at room temperature for 16 h. The mixture was quenched with water (80 mL) and extracted with ethyl acetate (60 mL×3). The combined organic extracts were dried over anhydrous Na₂SO₄, filtered, and concentrated under reduced pressure to give the product, which was purified by silica gel chromatography (eluent: petroleum ether:ethyl acetate=20:1 to 4:1) to afford the title compound (1.9 g, 91%) as a white solid. LCMS (ESI): mass calcd. for C₁₄H₁₂BrN₃O₂S 364.98 m/z, found 366.0 [M+H]⁺.

Step C. 2-Methyl-5-tosyl-7-(trimethylstannyl)-5H-pyrrolo[2,3-b]pyrazine. To a microwave vial containing 7-Bromo-2-methyl-5-tosyl-5H-pyrrolo[2,3-b]pyrazine (850 mg, 2.32 mmol) and 1,1,1,2,2,2-hexamethyldistannane (1.52 g, 4.64 mmol) was added toluene (10 mL). The mixture was sparged with Ar for 5 minutes and then treated with Pd(PPh₃)₄ (536 mg, 0.464 mmol). The mixture was sparged with Ar for another 5 minutes and then heated at 115° C. for 3 hours. The reaction mixture was cooled to room temperature, quenched with sat. KF (50 mL) and extracted with ethyl acetate (30 mL×3). The combined organic extracts were dried over anhydrous Na₂SO₄, filtered, and concentrated under reduced pressure to give the title compound (1.0 g) as a yellow oil. LCMS (ESI): mass calcd. for C₁₇H₂₁N₃O₂SSn 451.04 m/z, found 452.0 [M+H]⁺.

Step D. 4-Bromo-2-(2-methyl-5-tosyl-5H-pyrrolo[2,3-b]pyrazin-7-yl)thiazole. To a microwave vial containing 2-Methyl-5-tosyl-7-(trimethylstannyl)-5H-pyrrolo[2,3-b]pyrazine (1.0 g) and 2,4-dibromothiazole (1.08 g, 4.45 mmol) was added 1,4-dioxane (10 mL). The mixture was sparged with Ar for 5 minutes and then treated with Pd(Ph₃P)₄ (257 mg, 0.22 mmol). The mixture was sparged with Ar for another 5 minutes and then heated at 115° C. for 6 hours. The reaction mixture was cooled to room temperature, quenched with sat. KF (60 mL) and extracted with ethyl acetate (80 mL×3). The combined organic extracts were dried over anhydrous Na₂SO₄, filtered, and concentrated under reduced pressure to give the product, which was initially purified by silica gel chromatography (eluent: petroleum ether:ethyl acetate=20:1 to 3:1). The product was further purified by trituration with petroleum ether:ethyl acetate (3:1, 20 mL) and the suspension isolated via filtration. The filter cake was washed with petroleum ether (10 mL) and then dried under reduced pressure to afford the title compound (260 mg) as a brown sold. LCMS (ESI): mass calcd. for C₁₇H₁₃BrN₄O₂S₂ 447.97 m/z, found 449.0 [M+H]⁺.

Step E. (R)-7-(3-(2-(2-methyl-5-tosyl-5H-pyrrolo[2,3-b]pyrazin-7-yl)thiazol-4-yl)phenyl)-6,7-dihydro-5H-pyrrolo[1,2-a]imidazol-7-ol. To a microwave vial containing 4-Bromo-2-(2-methyl-5-tosyl-5H-pyrrolo[2,3-b]pyrazin-7-yl)thiazole (140 mg, 0.31 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 100 mg, 0.307 mmol) and K₃PO₄ (195 mg, 0.919 mmol) was added 1,4-dioxane (12 mL) and H₂O (3 mL). The mixture was sparged with Ar for 5 minutes and then treated with Pd(dtbpf)Cl₂ (20 mg, 0.031 mmol). The mixture was sparged with Ar for another 5 minutes and then heated at 80° C. via microwave irradiation for 1 hour. The mixture was filtered through a pad of diatomaceous earth and the pad was washed with ethyl acetate (30 mL). The combined organic extracts were concentrated under reduced pressure to give the product, which was purified by silica gel chromatography (eluent: petroleum ether:ethyl acetate=1:0 to 0:1, then, dichloromethane:methanol=1:0 to 10:1) to afford the title compound (80 mg, 41%) as a brown solid. LC-MS (ESI), mass calcd. For C₂₉H₂₄N₆O₃S₂ 568.14 m/z found 569.3 [M+H]⁺.

### Intermediate 142. (S)-7-(3-(2-(2-methyl-5-tosyl-5H-pyrrolo[2,3-b]pyrazin-7-yl)thiazol-4-yl)phenyl)-6,7-dihydro-5H-pyrrolo[1,2-a]imidazol-7-ol

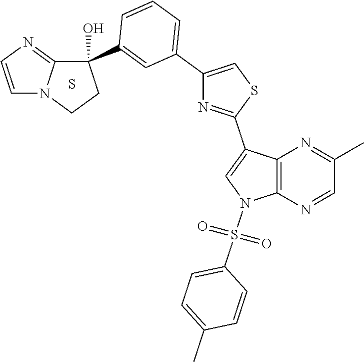

To a microwave vial containing 4-Bromo-2-(2-methyl-5-tosyl-5H-pyrrolo[2,3-b]pyrazin-7-yl)thiazole (Intermediate 141 Step D, 120 mg, 0.267 mmol), (S)-7-(3-(4,4,5,5-tetramethyl-1,3,2-dioxaborolan-2-yl)phenyl)-6,7-dihydro-5H-pyrrolo[1,2-a]imidazol-7-ol (Intermediate 39, 96 mg, 0.29 mmol) and K₃PO₄ (170 mg, 0.801 mmol) was added 1,4-dioxane (5 mL), and H₂O (1 mL). The mixture was sparged with Ar for 5 minutes and then treated with Pd(dtbpf)Cl₂ (17 mg, 0.03 mmol). The mixture was sparged with Ar for another 5 minutes and the resultant mixture was heated at 80° C. via microwave irradiation for 1 hour. After this time, the mixture was cooled to room temperature and concentrated under reduced pressure to give the product, which was purified by silica gel chromatography (eluent: petroleum ether:ethyl acetate=10:1 to 0:1, then dichloromethane:methanol=1:0 to 20:1) to afford the title compound (110 mg, 53%) as a brown solid. LCMS (ESI): mass calcd. for C₂₉H₂₄N₆O₃S₂ 568.14 m/z, found 569.1 [M+H]⁺.

### Intermediate 143. 4-bromo-2-(1H-pyrazolo[3,4-b]pyridin-3-yl)thiazole

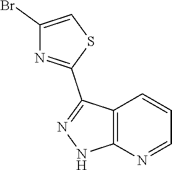 [SMILES: Brc1csc(-c2n[nH]c3ncccc23)n1]

Step A. (4-Bromothiazol-2-yl)(2 fluoropyridin-3-yl)methanone. Isopropylmagnesium chloride (4.5 mL, 2.0 M in THF, 9.0 mmol) was added to a 0° C. solution of 2,4-dibromothiazole (2.0 g, 8.2 mmol) and THF (20 mL). The resultant mixture was stirred at 0° C. for 2 hours. Then a solution of 2-fluoro-N-methoxy-N-methylnicotinamide (1.6 g, 8.7 mmol) and THF (5 mL) was added to the above solution at 0° C. The resultant mixture was stirred for 16 hours with gradual warming to room temperature. After this time, the mixture was poured into water (20 mL) and extracted with ethyl acetate (20 mL×3). The combined organic extracts were washed with brine (20 mL), dried over anhydrous Na₂SO₄, filtered, and concentrated to dryness under reduced pressure to give the product, which was purified by FCC (eluent: petroleum ether:ethyl acetate=1:0 to 2:1) to afford the title compound (1.5 g, 63%) as a yellow solid. ¹H NMR (400 MHz, DMSO-d₆) δ 8.57-8.51 (m, 2H), 8.49 (s, 1H), 7.64-7.58 (m, 1H).

Step B. (4-Bromothiazol-2-yl)(2-fluoropyridin-3-yl)methanone. Hydrazine monohydrate (1.3 mL, 27 mmol) was added to a solution of (4-bromothiazol-2-yl)(2-fluoropyridin-3-yl)methanone (1.5 g, 5.2 mmol) and THF (15 mL). The mixture was heated at 75° C. for 16 hours. After this time, the mixture was cooled to room temperature, poured into water (20 mL), and extracted with ethyl acetate (20 mL×3). The combined organic extracts were washed with brine (10 mL), dried over anhydrous Na₂SO₄, filtered, and concentrated to dryness under reduced pressure to afford the title compound (1.3 g, 88%) as a light yellow solid. ¹H NMR (400 MHz, DMSO-d₆) δ 8.64 (dd, J=1.5, 4.5 Hz, 1H), 8.60 (dd, J=1.5, 8.0 Hz, 1H), 7.90 (s, 1H), 7.39 (dd, J=4.5, 8.0 Hz, 1H).

### Intermediate 144. 4-Bromo-2-(1-tosyl-1H-pyrazolo[3,4-b]pyrazin-3-yl)thiazole

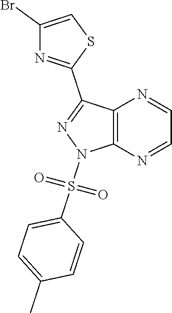 [SMILES: Cc1ccc(S(=O)(=O)n2nc(-c3nc(Br)cs3)c3nccnc32)cc1]

Step A. (4-Bromothiazol-2-yl)(3-chloropyrazin-2-yl)methanol. Isopropylmagnesium chloride (7.0 mL, 2.0 M in THF, 14 mmol) was added dropwise to a 0° C. mixture of 2,4-dibromothiazole (3.07 g, 12.6 mmol) and THF (20 mL). The resultant mixture was stirred at 0° C. for 2 hours, then treated with a solution of 3-chloropyrazine-2-carbaldehyde (1.8 g, 13 mmol) and THF (10 mL) dropwise, stirred at 0° C. for another 2 hours, and then stirred for 16 hours with gradual warming to room temperature. After this time, the suspension was poured into sat. NH₄Cl (30 mL) and extracted with ethyl acetate (70 mL×3). The combined organic extracts were dried over anhydrous Na₂SO₄, filtered, and concentrated to dryness under reduced pressure to give the product, which was purified by FCC (eluent: petroleum ether ethyl acetate=1:0 to 3:1) to afford the title compound (1 g, 26%) as a brown oil. LC-MS (ESI): mass calcd. For C₈H₅BrClN₃OS 304.90 m/z found 305.9 [M+H]⁺. ¹H NMR (400 MHz, DMSO-d₆) δ 8.68 (d, J=2.5 Hz, 1H), 8.55 (d, J=2.5 Hz, 1H), 7.86 (s, 1H), 7.39 (d, J=5.8 Hz, 1H), 6.35 (d, J=5.8 Hz, 1H).

Step B. (4-Bromothiazol-2-yl)(3-chloropyrazin-2-yl)methanone. Manganese(IV) oxide (1.4 g, 16 mmol) was added to a mixture of (4-bromothiazol-2-yl)(3-chloropyrazin-2-yl)methanol (1.0 g, 3.3 mmol) and methylene chloride (30 mL). The resultant mixture was stirred at room temperature for 12 hours. Then additional MnO₂ (0.5 g, 6 mmol) was added, and the resultant mixture was stirred at room temperature for another 2 hours. After this time, the mixture was filtered through a pad of diatomaceous earth and the pad was washed with methylene chloride (50 mL). The filtrate was concentrated to dryness under reduced pressure to give the product, which was purified by FCC (eluent: petroleum ether:ethyl acetate=1:0 to 3:1) to afford the title compound (820 mg, 83%) as a yellow solid. LC-MS (ESI): mass calcd. For C₈H₃BrClN₃OS 302.89 m/z found 304.0 [M+H]⁺. ¹H NMR (400 MHz, DMSO-d₆) δ 8.88-8.75 (m, 2H), 8.55 (s, 1H).

Step C. 4-Bromo-2-(1H-pyrazolo[3,4-b]pyrazin-3-yl)thiazole. Hydrazine monohydrate (0.62 mL, 13 mmol) was added to a solution of (4-bromothiazol-2-yl)(3-chloropyrazin-2-yl)methanone (770 mg, 2.53 mmol) and THE (10 mL). The resultant mixture was heated at 75° C. for 2 hours. After this time, the mixture was cooled to room temperature and filtered. The filter cake was washed with water (6 mL) and dried under reduced pressure to afford the title compound (600 mg, 84%) as a white solid. LC-MS (ESI): mass calcd. For C₈H₄BrN₅S 280.94 m/z, found 281.9 [M+H]⁺. ¹H NMR (400 MHz, DMSO-d₆) δ 14.72 (br s, 1H), 8.82 (d, J=2.0 Hz, 1H), 8.73 (d, J=2.0 Hz, 1H), 8.02 (s, 1H).

Step D. 4-Bromo-2-(1-tosyl-1H-pyrazolo[3,4-b]pyrazin-3-yl)thiazole. TosCl (94.5 mg, 0.496 mmol) was added to a solution of 4-bromo-2-(1H-pyrazolo[3,4-b]pyrazin-3-yl)thiazole (100 mg, 0.354 mmol), DMAP (8.7 mg, 0.071 mmol), dichloromethane (3 mL), and TEA (0.25 mL, 1.8 mmol). The reaction solution was stirred at room-temperature for 12 hours. The reaction mixture isolated via filtration. The filter cake was washed with dichloromethane (3 mL) before drying under reduced pressure to afford the title compound (100 mg) as a white solid.

### Intermediate 145. (R)-7-(3-(2-(3-methyl-5-tosyl-5H-pyrrolo[2,3-b]pyrazin-7-yl)thiazol-4-yl)phenyl)-6,7-dihydro-5H-pyrrolo[1,2-a]imidazol-7-ol

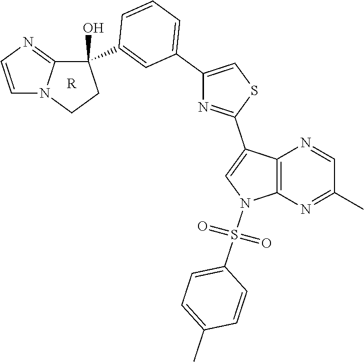

Step A. 3-Bromo-5-tosyl-5H-pyrrolo[2,3-b]pyrazine. To a flask containing dichloromethane (20 mL) was added TosCl (2.3 g, 12 mmol), 3-bromo-5H-pyrrolo[2,3-b]pyrazine (2.0 g, 10 mmol), DMAP (247 mg, 2.02 mmol) and Et₃N (7.0 ml, 50 mmol). The resultant mixture was stirred at room temperature for 16 hours before quenching with water (50 mL) and extracting with dichloromethane (50 mL×3). The combined organic extracts were washed with brine (10 mL), dried over anhydrous Na₂SO₄, filtered, and concentrated under reduced pressure to give the product, which was purified by silica gel chromatography (eluent: petroleum ether:ethyl acetate=20:1 to 3:1) to afford the title compound (3.55 g, 75%) as a brown solid. LC-MS (ESI): mass calcd. For C₁₃H₁₀BrN₃O₂S 350.97 m/z found 352.0 [M+H]⁺. ¹H NMR (400 MHz, DMSO-d₆) δ 8.75 (s, 1H), 8.30 (d, J=4.2 Hz, 1H), 7.99 (d, J=8.2 Hz, 2H), 7.47 (d, J=8.2 Hz, 2H), 7.05 (d, J=4.2 Hz, 1H), 2.36 (s, 3H).

Step B. 3-Methyl-5-tosyl-5H-pyrrolo[2,3-b]pyrazine. 3-Bromo-5-tosyl-5H-pyrrolo[2,3-b]pyrazine (3.0 g, 8.5 mmol), 2,4,6-trimethyl-1,3,5,2,4,6-trioxatriborinane (3.57 mL, 25.5 mmol), K₃PO₄ (2.94 g, 21.3 mmol), and 1,4-dioxane (20 mL) were added to a 250 mL three-necked round bottom flask. The mixture was sparged with Ar for 5 minutes and then treated with Pd(dppf)Cl₂ (623 mg, 0.851 mmol). The mixture was sparged with Ar for another 5 minutes and then heated at 115° C. for 16 hours. After this time, the mixture was cooled to room temperature, poured into water (50 mL), and extracted with dichloromethane (50 mL×3). The combined organic extracts were washed with brine (10 mL), dried over anhydrous Na₂SO₄, filtered, and concentrated to dryness under reduced pressure to give the product, which was purified by silica gel chromatography (eluent: petroleum ether:ethyl acetate=20:1 to 5:1) to afford the title compound (2.3 g, 89%) as a brown solid. LC-MS (ESI): mass calcd. For C₁₄H₁₃N₃O₂S 287.07 m/z found 288.0 [M+H]⁺. ¹H NMR (400 MHz, DMSO-d₆) δ 8.50 (s, 1H), 8.15 (d, J=4.0 Hz, 1H), 8.01 (d, J=8.4 Hz, 2H), 7.43 (d, J=8.2 Hz, 2H), 6.95 (d, J=4.2 Hz, 1H), 2.59 (s, 3H), 2.34 (s, 3H).

Step C. 7-Bromo-3-methyl-5-tosyl-5H-pyrrolo[2,3-b]pyrazine. A flask containing a DMF (30 mL) solution of 3-methyl-5-tosyl-5H-pyrrolo[2,3-b]pyrazine (2.3 g, 8.0 mmol) was cooled to 0° C. (ice/H₂O) and NBS (1.43 g, 8.03 mmol) was then added. The resultant mixture was stirred for 1 hour with gradual warming to room temperature, and then the resultant mixture was stirred at room temperature for 16 hours. The mixture was quenched with water (30 mL) and extracted with dichloromethane (100 mL×3). The combined organic extracts were washed with brine (10 mL), dried over anhydrous Na₂SO₄, filtered, and concentrated to dryness under reduced pressure to give the product, which was purified by silica gel chromatography (eluent: petroleum ether:ethyl acetate=20:1 to 5:1) to afford the title compound (2.6 g, 78%) as a white solid. LC-MS (ESI): mass calcd. For C₁₄H₁₂BrN₃O₂S 364.98 m/z found 366.0 [M+H]⁺.

Step D. 3-Methyl-5-tosyl-7-(trimethylstannyl)-5H-pyrrolo[2,3-b]pyrazine. To a flask containing 7-bromo-3-methyl-5-tosyl-5H-pyrrolo[2,3-b]pyrazine (1.5 g, 4.1 mmol), was added toluene (20 mL) and 1,1,1,2,2,2-hexamethyldistannane (2.670 g, 8.150 mmol). The mixture was sparged with Ar for 5 minutes and then treated with Pd(PPh₃)₄ (947 mg, 0.820 mmol). The mixture was sparged with Ar for another 5 minutes and then heated at 115° C. for 5 hours. The reaction mixture was cooled to room temperature, quenched with sat. KF (200 mL) and extracted with ethyl acetate (100 mL×3). The combined organic extracts were dried over anhydrous Na₂SO₄, filtered, and concentrated to dryness under reduced pressure to afford the product (1.8 g) as a yellow oil. LC-MS (ESI): mass calcd. For C₁₇H₂₁N₃O₂SSn 450.143 m/z found 451.9 [M+H]⁺.

Step E. 4-Bromo-2-(3-methyl-5-tosyl-5H-pyrrolo[2,3-b]pyrazin-7-yl)thiazole. To a flask containing 3-methyl-5-tosyl-7-(trimethylstannyl)-5H-pyrrolo[2,3-b]pyrazine (1.8 g) and 2,4-dibromothiazole (1.94 g, 7.99 mmol) was added 1,4-dioxane (30 mL). The mixture was sparged with Ar for 5 minutes and then treated with Pd(Ph₃P)₄ (462 mg, 0.400 mmol). The mixture was sparged with Ar for another 5 minutes and then heated at 115° C. for 16 hours. The reaction mixture was cooled to room temperature, quenched with sat. KF (80 mL) and extracted with ethyl acetate (100 mL×3). The combined organic extracts were dried over anhydrous Na₂SO₄, filtered, and concentrated to dryness under reduced pressure to give the product, which was initially purified by silica gel chromatography (eluent: petroleum ether:ethyl acetate=20:1 to 3:1). The product was further purified by trituration with ethyl acetate:DMSO (10:1, 20 mL) and the solids isolated via filtration. The filter cake was washed with petroleum ether (10 mL) and then dried under reduced pressure to afford the title compound (300 mg, 17%) as a brown sold. LC-MS (ESI): mass calcd. For C₁₇H₁₃BrN₄O₂S₂ 449.97 m/z found 450.9 [M+H]⁺.

Step F. (R)-7-(3-(2-(3-Methyl-5-tosyl-5H-pyrrolo[2,3-b]pyrazin-7-yl)thiazol-4-yl)phenyl)-6,7-dihydro-5H-pyrrolo[1,2-a]imidazol-7-ol. To a microwave vial containing 4-bromo-2-(3-methyl-5-tosyl-5H-pyrrolo[2,3-b]pyrazin-7-yl)thiazole (130 mg, 0.289 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 104 mg, 0.319 mmol) and K₃PO₄ (118 mg, 0.867 mmol) was added 1,4-dioxane (6 mL), and H₂O (1.5 mL). The mixture was sparged with Ar for 5 minutes and then treated with Pd(dtbpf)Cl₂ (19 mg, 0.029 mmol). The mixture was sparged with Ar for another 5 minutes and the resultant mixture was heated at 85° C. via microwave irradiation for 1 hour. The reaction mixture was cooled to room temperature and then concentrated to dryness under reduced pressure to give the product. The product was initially was purified by silica gel chromatography (eluent: petroleum ether ethyl acetate=10:1 to 0:1, and then subjected to a second silica gel chromatography column with dichloromethane:methanol=1:0 to 20:1 as the eluent to afford the title compound (110 mg, 67%) as a brown solid. LC-MS (ESI): mass calcd. For C₂₉H₂₄N₆O₃S₂ 568.14 m/z found 569.1 [M+H]⁺.

### Intermediate 146. (S)-7-(3-(4-(1-(phenylsulfonyl)-1H-pyrrolo[2,3-b]pyridin-3-yl)thiazol-2-yl)phenyl)-6,7-dihydro-5H-pyrrolo[1,2-a]imidazol-7-ol

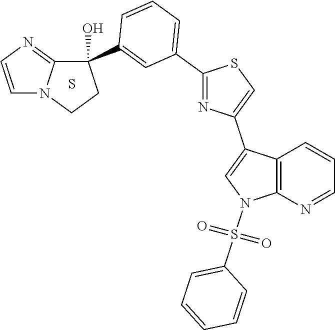

Step A. (S)-7-(3-(4-Bromothiazol-2-yl)phenyl)-6,7-dihydro-5H-pyrrolo[1,2-a]imidazol-7-ol. To a microwave vial containing (S)-7-(3-(4,4,5,5-Tetramethyl-1,3,2-dioxaborolan-2-yl)phenyl)-6,7-dihydro-5H-pyrrolo[1,2-a]imidazol-7-ol (Intermediate 39, 220 mg, 0.674 mmol), 2,4-dibromothiazole (246 mg, 1.01 mmol) and Cs₂CO₃ (549 mg, 1.69 mmol) was added 1,4-dioxane (10 mL), and H₂O (2 mL). The mixture was sparged with Ar for 5 minutes and then treated with Pd(dppf)Cl₂ (49 mg, 0.07 mmol). The mixture was sparged with Ar for another 5 minutes and then heated at 80° C. via microwave irradiation for 1 hour. After this time, the mixture was cooled to room temperature and concentrated to dryness under reduced pressure to give the product, which was purified by silica gel chromatography (eluent: petroleum ether:ethyl acetate=5:1 to 0:1) to afford the title compound (190 mg, 47%) as a brown solid. LCMS (ESI): mass calcd. for C₁₅H₁₂BrN₃OS 360.99 m/z, found 362.0 [M+H]⁺. ¹H NMR (400 MHz, DMSO-d₆) δ 8.12-8.08 (m, 1H), 7.91 (s, 1H), 7.86-7.82 (m, 1H), 7.53-7.48 (m, 2H), 7.22-7.16 (m, 1H), 7.04-7.01 (m, 1H), 6.28 (s, 1H), 4.15-4.01 (m, 2H), 2.85-2.76 (m, 2H).

Step B. (S)-7-(3-(4-(1-(Phenylsulfonyl)-1H-pyrrolo[2,3-b]pyridin-3-yl)thiazol-2-yl)phenyl)-6,7-dihydro-5H-pyrrolo[1,2-a]imidazol-7-ol. To a microwave vial containing (S)-7-(3-(4-Bromothiazol-2-yl)phenyl)-6,7-dihydro-5H-pyrrolo[1,2-a]imidazol-7-ol (210 mg, 0.580 mmol), 1-(phenylsulfonyl)-3-(4,4,5,5-tetramethyl-1,3,2-dioxaborolan-2-yl)-1H-pyrrolo[2,3-b]pyridine (290 mg, 0.755 mmol) and K₃PO₄ (369 mg, 1.74 mmol) was added 1,4-dioxane (10 mL), and H₂O (2 mL). The mixture was sparged with Ar for 5 minutes and then treated with Pd(dtbpf)Cl₂ (38 mg, 0.06 mmol). The mixture was sparged with Ar for another 5 minutes and then heated at 80° C. via microwave irradiation for 1 hour. After this time, the mixture was cooled to room temperature and concentrated to dryness under reduced pressure to give the product, which was purified by silica gel chromatography (eluent: petroleum ether:ethyl acetate=10:1 to 0:1) to afford the title compound (120 mg, 36%) as a brown solid. LCMS (ESI): mass calcd. for C₂₈H₂₁N₅O₃S₂ 539.11 m/z, found 540.1 [M+H]⁺.

### Intermediate 147. (R)-3-(3-bromophenyl)-2,3-dihydrofuro[3,2-b]pyridin-3-ol

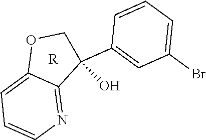

Step A. Ethyl 3-hydroxypicolinate. 3-Hydroxypicolinic acid (30.0 g, 216 mmol) was added to a solution of EtOH (360 mL, 6.17 mol), H₂SO₄ (10.0 mL), and toluene (100 mL). The resultant mixture was heated at 78° C. for 48 hours and then cooled to room temperature, quenched with saturated aqueous NaHCO₃ solution (50 mL) and water (150 mL), and extracted with ethyl acetate (200 mL×3). The combined organic extracts were dried over anhydrous Na₂SO₄, filtered, and concentrated to dryness under reduced pressure to afford the product, which was purified by silica gel chromatography (eluent: petroleum ether:ethyl acetate=10:1 to 5:1) to afford title compound (23.8 g, 63%) as a colorless oil. LC-MS (ESI): mass calcd. For C₆H₉NO₃ 167.06 m/z found 168.1 [M+H]⁺.

Step B. Ethyl 3-(2-ethoxy-2-oxoethoxy)picolinate. Ethyl 2-bromoacetate (18.1 mL, 163 mmol) was added to a solution of ethyl 3-hydroxypicolinate (23.8 g, 142 mmol), K₂CO₃ (59.0 g, 427 mmol), and acetone (250 mL). The reaction mixture was stirred at 60° C. for 15 hours and then cooled to room temperature. The reaction mixture was filtered and concentrated to dryness under reduced pressure to give the product, which was purified by silica gel chromatography (eluent: petroleum ether:ethyl acetate=10:1 to 4:1) to afford title compound (22 g, 61%) as a yellow oil. ¹H NMR (400 MHz, DMSO-d₆) δ 8.23-8.16 (m, 1H), 7.58-7.48 (m, 2H), 4.92 (s, 2H), 4.35-4.29 (m, 2H), 4.16 (q, J=7.1 Hz, 2H), 1.30 (t, J=7.1 Hz, 3H), 1.20 (t, J=7.1 Hz, 3H).

Step C. Ethyl 3-hydroxyfuro[3,2-b]pyridine-2-carboxylate. Ethyl 3-(2-ethoxy-2-oxoethoxy)picolinate (22 g, 87 mmol) was added to a solution of sodium ethanolate (71.2 mL, 21% in EtOH, 191 mmol) and toluene (150 mL). The reaction mixture was heated at 111° C. for 18 hours and then allowed to cool to room temperature. The pH was adjusted to pH between 7-8 with 12 M HCl, and the mixture was extracted with ethyl acetate (150 mL×3). The combined organic extracts were dried over anhydrous Na₂SO₄, filtered, and concentrated to dryness under reduced pressure to give the product, which was purified by silica gel chromatography (eluent: petroleum ether ethyl acetate=1:0 to 1:2) to afford the title compound (8.6 g, 48%) as a white solid. ¹H NMR (400 MHz, DMSO-d₆) δ 11.73 10.90 (m, 1H), 8.63 (dd, J=1.1, 4.4 Hz, 1H), 8.07 (dd, J=1.1, 8.4 Hz, 1H), 7.55 (dd, J=4.6, 8.6 Hz, 1H), 4.32 (q, J=7.2 Hz, 2H), 1.31 (t, J=7.2 Hz, 3H).

Step D. Furo[3,2-b]pyridin-3(2H)-one. Ethyl 3-hydroxyfuro[3,2-b]pyridine-2-carboxylate (3.00 g, 14.5 mmol) and aqueous HCl (3 M, 120 mL) were added to a 500 mL round bottom flask. The resultant mixture was heated at 100° C. for 5 hours and then allowed to cool to room temperature. The pH was adjusted to pH between 6-7 with 1M HCl, and the mixture was extracted with ethyl acetate (100 mL×3). The combined organic extracts were concentrated to dryness under reduced pressure to afford compound (1.8 g) as brown oil, which was used next step without further purification. ¹H NMR (400 MHz, DMSO-d₆) δ 8.53-8.49 (m, 1H), 7.82-7.79 (m, 1H), 7.69-7.64 (m, 1H), 4.89 (s, 2H).

Step E. (R)-3-(3-Bromophenyl)-2,3-dihydrofuro[3,2-b]pyridin-3-ol. n-BuLi (11.7 mL, 2.5 M in hexane, 29.3 mmol) was added drop wise to a −70° C. (dry ice/EtOH) solution of 1,3-dibromobenzene (6.9 g, 29 mmol) and THF (40 mL). The resultant mixture was stirred at −70° C. for 30 min before adding a solution of furo[3,2-b]pyridin-3(2H)-one (1.8 g) and THF (2 mL). The mixture was stirred for 16 hours with gradual warming to room temperature. The mixture was quenched with sat. NH₄Cl (80 mL) and extracted with ethyl acetate (80 mL×3). The combined organic extracts were dried over anhydrous Na₂SO₄, filtered, and concentrated to dryness under reduced pressure. The product was initially purified by silica gel chromatography (eluent: petroleum ether ethyl acetate=10:1 to 2:1) and then further purified by HPLC using a Xtimate C18, 150×40 mm×10 μm column (eluent: 25% to 55% (v/v) CH₃CN and H₂O with 0.04% NH₃H₂O+10 mM NH₄HCO₃) to afford the title compound (190 mg, 5%) as a brown oil. The (R) and (S) enantiomers of racemic 3-(3-Bromophenyl)-2,3-dihydrofuro[3,2-b]pyridin-3-ol (260 mg, 0.890 mmol) were separated by SFC over Phenomenex-Cellulose-2 250 mm×30 mm, 10 μm (isocratic elution:EtOH (containing 0.1% of 25% aq. NH₃): supercritical CO₂=25%: 75% (v/v)). The first eluting enantiomer (R), the title compound, was obtained as a clear oil (110 mg, 41%). LCMS (ESI): mass calcd. for C₁₃H₁₀BrNO₂ 290.99 m/z, found 292.1 [M+H]⁺. The second eluting enantiomer (S) was obtained as a clear oil (110 mg, 41%). LCMS (ESI): R_{T}=0.83 min, mass calcd. for C₁₃H₁₀BrNO₂ 290.99 m/z, found 292.0 [M+H]⁺.

### Intermediate 148. (R)-3-(3-(2-(1-(phenylsulfonyl)-1H-pyrrolo[2,3-b]pyridin-3-yl)thiazol-4-yl)phenyl)-2,3-dihydrofuro[3,2-b]pyridin-3-ol

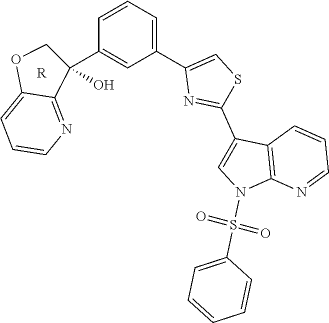

Step A. (R)-3-(3-(4,4,5,5-Tetramethyl-1,3,2-dioxaborolan-2-yl)phenyl)-2,3-dihydrofuro[3,2-b]pyridin-3-ol. To a microwave vial containing (R)-3-(3-Bromophenyl)-2,3-dihydrofuro[3,2-b]pyridin-3-ol (Intermediate 147, 100 mg, 0.342 mmol), 4,4,4′,4′,5,5,5′,5′-octamethyl-2,2′-bi(1,3,2-dioxaborolane) (139 mg, 0.547 mmol) and KOAc (101 mg, 1.03 mmol) was added 1,4-dioxane (10 mL). The mixture was sparged with N₂ for 5 minutes and then treated with Pd(dppf)Cl₂ (25 mg, 0.03 mmol). The resultant mixture was heated at 85° C. via microwave irradiation for 1 hour. The mixture was cooled to room temperature and concentrated to dryness under reduced pressure to give the product, which was purified by silica gel chromatography (eluent: petroleum ether:ethyl acetate=10:1 to 1:2) to afford the title compound (110 mg, 75%) as a yellow oil. LCMS (ESI): mass calcd. for C₁₉H₂₂BNO₄ 339.16 m/z, found 340.2 [M+H]⁺.

Step B. (R)-3-(3-(2-(1-(Phenylsulfonyl)-1H-pyrrolo[2,3-b]pyridin-3-yl)thiazol-4-yl)phenyl)-2,3-dihydrofuro[3,2-b]pyridin-3-ol. To a microwave vial containing 4-Bromo-2-(1-(phenylsulfonyl)-1H-pyrrolo[2,3-b]pyridin-3-yl)thiazole (Intermediate 2, 136 mg, 0.324 mmol), (R)-3-(3-(4,4,5,5-tetramethyl-1,3,2-dioxaborolan-2-yl)phenyl)-2,3-dihydrofuro[3,2-b]pyridin-3-ol (110 mg, 0.324 mmol) and K₃PO₄ (207 mg, 0.975 mmol) was added 1,4-dioxane (10 mL), and H₂O (2 mL). The mixture was sparged with Ar for 5 minutes and then treated with Pd(dtbpf)Cl₂ (21 mg, 0.03 mmol). The mixture was sparged with Ar for another 5 minutes and the resultant mixture was heated at 80° C. via microwave irradiation for 1 hour. The reaction mixture was cooled to room temperature and concentrated to dryness under reduced pressure to give the product, which was purified by silica gel chromatography (eluent: petroleum ether ethyl acetate=10:1 to 0:1) to afford the title compound (120 mg, 61%) as a brown solid. LCMS (ESI), mass calcd. for C₂₉H₂₀N₄O₄S₂ 552.09 m/z, found 553.1 [M+H]⁺.

### Intermediate 149. 4-bromo-2-(5H-pyrrolo[2,3-b]pyrazin-7-yl)thiazole

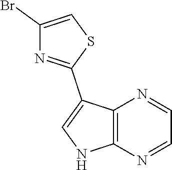 [SMILES: Brc1csc(-c2c[nH]c3nccnc23)n1]

The title compound was obtained from the synthesis described for intermediate 10. LCMS (ESI), mass calcd. for C₉H₅BrN₄S 279.94 m/z, found 282.9 [M+H]⁺.

### Intermediate 150. (R)-3-(3-(2-(5-tosyl-5H-pyrrolo[2,3-b]pyrazin-7-yl)thiazol-4-yl)phenyl)-2,3-dihydrofuro[3,2-b]pyridin-3-ol

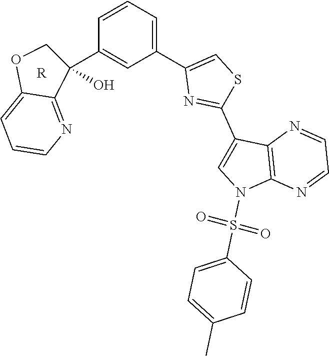

To a microwave vial containing 4-Bromo-2-(5-tosyl-5H-pyrrolo[2,3-b]pyrazin-7-yl)thiazole (Intermediate 10, 116 mg, 0.266 mmol), (R)-3-(3-(4,4,5,5-tetramethyl-1,3,2-dioxaborolan-2-yl)phenyl)-2,3-dihydrofuro[3,2-b]pyridin-3-ol (Intermediate 148, Step A, 90 mg, 0.27 mmol) and K₃PO₄ (169 mg, 0.796 mmol) was added 1,4-dioxane (10 mL), and H₂O (2 mL). The mixture was sparged with Ar for 5 minutes and then treated with Pd(dtbpf)Cl₂ (17 mg, 0.03 mmol). The mixture was sparged with Ar for another 5 minutes and the resultant mixture was heated at 85° C. via microwave irradiation for 1 hour. The reaction mixture was cooled to room temperature and concentrated to dryness under reduced pressure to give the product, which was purified by silica gel chromatography (eluent: petroleum ether:ethyl acetate=10:1 to 0:1) to afford the title compound (100 mg, 52%) as a brown solid. LCMS (ESI): mass calcd. for C₂₉H₂₁N₅O₄S₂ 567.10 m/z, found 568.1 [M+H]⁺.

### Intermediate 151. (R)-7-(3-(2-(4-methyl-1-tosyl-1H-pyrrolo[2,3-b]pyridin-3-yl)thiazol-4-yl)phenyl)-6,7-dihydro-5H-pyrrolo[1,2-a]imidazol-7-ol

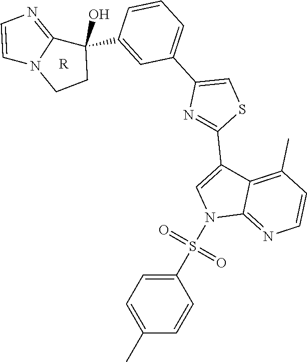

Step A. 3-Bromo-4-methyl-1-tosyl-1H-pyrrolo[2,3-b]pyridine. TosCl (470 mg, 2.47 mmol) was added to a solution of 3-bromo-4-methyl-1H-pyrrolo[2,3-b]pyridine (400 mg, 1.90 mmol), Et₃N (1.0 mL, 7.2 mmol), DMAP (46 mg, 0.38 mmol), and dichloromethane (15 mL). The resultant mixture was stirred at room temperature for 12 hours before pouring it into H₂O (40 mL) and extracting with ethyl acetate (50 mL×3). The combined organic extracts were washed with brine (10 mL), dried over anhydrous Na₂SO₄, filtered, and concentrated to dryness under reduced pressure to give the product, which was purified by silica gel chromatography (eluent: petroleum ether:ethyl acetate=1:0 to 0:1) to afford the title compound (600 mg, 86%) as a yellow solid. LC-MS (ESI): mass calcd. For C₁₅H₁₃BrN₂O₂S 363.99 m/z found 365.0 [M+H]⁺.

Step B. 4-Methyl-3-(4,4,5,5-tetramethyl-1,3,2-dioxaborolan-2-yl)-1-tosyl-1H-pyrrolo[2,3-b]pyridine. 3-Bromo-4-methyl-1-tosyl-1H-pyrrolo[2,3-b]pyridine (600 mg, 1.64 mmol) was added to a mixture 4,4,4′,4′,5,5,5′,5′-octamethyl-2,2′-bi(1,3,2-dioxaborolane) (1.05 g, 4.14 mmol), KOAc (484 mg, 4.93 mmol), and 1,4-dioxane (20 mL). The mixture was sparged with Ar for 5 minutes and then treated with Pd(OAc)₂ (36.9 mg, 0.164 mmol) and PCy₃ (92 mg, 0.33 mmol). The mixture was heated at 70° C. for 5 hours under N₂ atmosphere. The reaction mixture cooled to room temperature and combined with another experiment run on the same scale. The combined mixture was filtered through a pad of diatomaceous earth and the pad was washed with ethyl acetate (40 mL). The filtrate was concentrated to dryness under reduced pressure to give the product, which was purified by silica gel chromatography (eluent: petroleum ether:ethyl acetate=1:0 to 1:1) to afford the title compound (450 mg, 62%) as a brown solid. LC-MS (ESI): mass calcd. For C₂₁H₂₅BN₂O₄S 412.16 m/z found 413.2 [M+H]⁺.

Step C. 4-Bromo-2-(4-methyl-1-tosyl-1H-pyrrolo[2,3-b]pyridin-3-yl)thiazole. To a microwave vial containing 4-methyl-3-(4,4,5,5-tetramethyl-1,3,2-dioxaborolan-2-yl)-1-tosyl-1H-pyrrolo[2,3-b]pyridine (420 mg, 1.02 mmol), 2,4-dibromothiazole (356 mg, 1.47 mmol) and Cs₂CO₃ (775 mg, 2.38 mmol) was added 1,4-dioxane (15 mL), and H₂O (3 mL). The mixture was sparged with Ar for 5 minutes and then treated with Pd(dppf)Cl₂ (77.5 mg, 0.106 mmol). The mixture was sparged with Ar for another 5 minutes and then heated at 80° C. via microwave irradiation for 1 hour. The reaction mixture was cooled to room temperature, concentrated to dryness under reduced pressure to give the product, which was purified by silica gel chromatography (eluent: petroleum ether:ethyl acetate=1:0 to 0:1) to afford the title compound (280 mg, 34%) as a brown solid. LC-MS (ESI): mass calcd. For C₁₈H₁₄BrN₃O₂S₂ 446.97 m/z found 448.8 [M+H]⁺.

Step D. (R)-7-(3-(2-(4-Methyl-1-tosyl-1H-pyrrolo[2,3-b]pyridin-3-yl)thiazol-4-yl)phenyl)-6,7-dihydro-5H-pyrrolo[1,2-a]imidazol-7-ol. 4-Bromo-2-(4-methyl-1-tosyl-1H-pyrrolo[2,3-b]pyridin-3-yl)thiazole (200 mg, 0.250 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 100 mg, 0.307 mmol), K₃PO₄ (156 mg, 0.735 mmol), 1,4-dioxane (12 mL), and H₂O (3 mL) were added to a microwave tube. The mixture was sparged with Ar for 5 minutes and then treated with Pd(dtbpf)Cl₂ (16 mg, 0.025 mmol). The mixture was sparged with Ar for another 5 minutes and then heated at 80° C. via microwave irradiation for 1 hour. The reaction mixture cooled to room temperature and concentrated to dryness under reduced pressure to give the product, which was purified by two silica gel chromatography columns (1) eluent: petroleum ether:ethyl acetate=1:0 to 0:1, then, 2) ethyl acetate:methanol=1:0 to 5:1) to afford the title compound (150 mg, 92%) as a brown solid. LC-MS (ESI): mass calcd. For C₃₀H₂₅N₅O₃S₂ 567.14 m/z found 568.2 [M+H]⁺.

### Intermediate 152. (R)-7-(3-(2-(7-tosyl-7H-pyrrolo[2,3-c]pyridazin-5-yl)thiazol-4-yl)phenyl)-6,7-dihydro-5H-pyrrolo[1,2-a]imidazol-7-ol

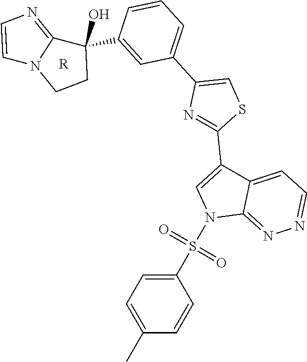

Step A. 5-Bromo-7H-pyrrolo[2,3-c]pyridazine. N-Bromosuccinimide (164 mg, 0.921 mmol) was added to a solution of 7H-pyrrolo[2,3-c]pyridazine (100 mg, 0.839 mmol) and THF (3 mL). The resultant mixture was stirred at room temperature for 16 hours. After this time, the mixture concentrated to dryness under reduced pressure afford the product, which was purified by FCC (eluent: petroleum ether:ethyl acetate=5:1 to 0:1) to afford the title compound (120 mg, 72%) as a yellow solid. ¹H NMR (400 MHz, DMSO-d₆) δ 12.87 (br s, 1H), 8.97 (d, J=5.5 Hz, 1H), 8.16 (s, 1H), 7.76 (d, J=5.5 Hz, 1H).

Step B. 5-Bromo-7-tosyl-7H-pyrrolo[2,3-c]pyridazine. 4-Toluenesulfonyl chloride (1.0 g, 5.2 mmol) was added to a solution of 5-bromo-7H-pyrrolo[2,3-c]pyridazine (800 mg, 4.04 mmol), DMAP (99 mg, 0.81 mmol), Et₃N (2.8 ml, 20 mmol), and methylene chloride (15 mL). The resultant mixture was stirred at room temperature for 16 hours. After this time, the mixture was concentrated to dryness under reduced pressure to give the product, which was purified by FCC (eluent: petroleum ether:ethyl acetate=20:1 to 3:1) to afford the compound (1.1 g, 77%) as a white solid. LCMS (ESI): R_{T}=0.80 min, mass calcd. for C₁₃H₁₀BrN₃O₂S 350.97 m/z, found 352.0 [M+H]⁺.

Step C. 5-(4,4,5,5-Tetramethyl-1,3,2-dioxaborolan-2-yl)-7-tosyl-7H-pyrrolo[2,3-c]pyridazine. 5-Bromo-7-tosyl-7H-pyrrolo[2,3-c]pyridazine (500 mg, 1.42 mmol), 4,4,4′,4′,5,5,5′,5′-octamethyl-2,2′-bi(1,3,2-dioxaborolane) (721 mg, 2.84 mmol), KOAc (418 mg, 4.26 mmol), and THF (15 mL) were added to a 100 mL round-bottomed flask. The mixture was sparged with N₂ for 5 minutes and then treated with Pd(dppf)Cl₂ (104 mg, 0.142 mmol). The resultant mixture was heated at 70° C. for 3 hours. After this time, the mixture was cooled to room temperature, filtered through a pad of diatomaceous earth, and the pad was washed with ethyl acetate (20 mL). The filtrate was concentrated to dryness under reduced pressure to afford the title compound (850 mg) as a yellow oil, which was used in the next step without purification. LCMS (ESI): mass calcd. for C₁₉H₂₂BN₃O₄S 399.14 m/z, found 318.1 [M−82+H]⁺.

Step D. 4-Bromo-2-(7-tosyl-7H-pyrrolo[2,3-c]pyridazin-5-yl)thiazole. To a microwave vial containing 5-(4,4,5,5-Tetramethyl-1,3,2-dioxaborolan-2-yl)-7-tosyl-7H-pyrrolo[2,3-c]pyridazine (800 mg), 2,4-dibromothiazole (487 mg, 2.00 mmol) and Cs₂CO₃ (1.31 g, 4.02 mmol) was added 1,4-dioxane (10 mL), and H₂O (2 mL). The mixture was sparged with Ar for 5 minutes and then treated with Pd(dppf)Cl₂ (147 mg, 0.201 mmol). The mixture was sparged with Ar for another 5 minutes and then heated at 80° C. via microwave irradiation for 1 hour. After this time, the mixture was cooled to room temperature and concentrated to dryness under reduced pressure to give the product, which was purified by FCC (eluent: petroleum ether:ethyl acetate=10:1 to 2:1) to afford the title compound (130 mg, 12%) as a brown solid. LCMS (ESI): mass calcd. for C₁₆H₁₁BrN₄O₂S₂ 433.95 m/z, found 435.0 [M+H]⁺.

Step E. (R)-7-(3-(2-(7-Tosyl-7H-pyrrolo[2,3-c]pyridazin-5-yl)thiazol-4-yl)phenyl)-6,7-dihydro-5H-pyrrolo[1,2-a]imidazol-7-ol. To a microwave vial containing 4-Bromo-2-(7-tosyl-7H-pyrrolo[2,3-c]pyridazin-5-yl)thiazole (130 mg, 0.299 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 97 mg, 0.30 mmol) and K₃PO₄ (190 mg, 0.90 mmol) was added 1,4 dioxane (10 mL), and H₂O (2 mL). The mixture was sparged with Ar for 5 minutes and then treated with Pd(dtbpf)Cl₂ (19 mg, 0.03 mmol). The mixture was sparged with Ar for another 5 minutes and then heated at 80° C. via microwave irradiation for 1 hour. After this time, the mixture was cooled to room temperature. The mixture was concentrated to dryness under reduced pressure to give the product, which was purified by FCC. The column was first eluted using 90% petroleum ether with an increasing gradient to 100% EtOAc, and then further increasing the gradient from 100% methylene chloride to 10% MeOH-methylene chloride to give the title compound (120 mg, 72%) as a brown solid. LCMS (ESI): mass calcd. for C₂₈H₂₂N₆O₃S₂ 554.12 m/z, found 555.1 [M+H]⁺.

### Intermediate 153. 3-Hydroxy-3-(3-(2-(1-(phenylsulfonyl)-1H-pyrrolo[2,3-b]pyridin-3-yl)thiazol-4-yl)phenyl)-1,3-dihydro-2H-pyrrolo[3,2-b]pyridin-2-one

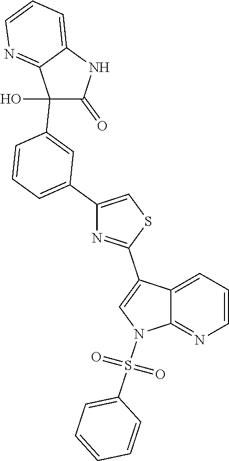 [SMILES: O=C1Nc2cccnc2C1(O)c1cccc(-c2csc(-c3cn(S(=O)(=O)c4ccccc4)c4ncccc34)n2)c1]

Step A. 4-Bromo-2-(5-fluoro-1-tosyl-1H-pyrrolo[2,3-b]pyridin-3-yl)thiazole. NaH (162 mg, 60% purity, 4.05 mmol) was added to a 0° C. solution of 1H-pyrrolo[3,2-b]pyridine-2,3-dione (500 mg, 3.38 mmol) and THF (15 mL). The mixture was stirred at 0° C. for 30 mins. Then, (3-chlorophenyl)magnesium bromide (14 mL, 0.5 M in THF, 7.0 mmol) was added to this mixture at 0° C. The resultant mixture was stirred for 16 hours with gradual warming to room-temperature. The mixture was quenched with sat. NH₄Cl (15 mL) and extracted with ethyl acetate (15 mL×3). The combined organic extracts were washed with brine (15 mL), dried over anhydrous Na₂SO₄, filtered, and concentrated to dryness under reduced pressure to give the product, which was purified by silica gel chromatography (eluent: petroleum ether:ethyl acetate=1:0 to 1:2) to afford the title compound (120 mg, 11%) as a yellow solid. LC-MS (ESI): mass calcd. For C₁₃H₉ClN₂O₂ 260.04 m/z found 261.0 [M+H]⁺.

Step B. 3-Hydroxy-3-(3-(4,4,5,5-tetramethyl-1,3,2-dioxaborolan-2-yl)phenyl)-1H-pyrrolo[3,2-b]pyridin-2(3H)-one. KOAc (315 mg, 3.21 mmol) was added to a mixture of 3-(3-chlorophenyl)-3-hydroxy-1H-pyrrolo[3,2-b]pyridin-2(3H)-one (280 mg, 1.07 mmol), 4,4,4′,4′,5,5,5′,5′-octamethyl-2,2′-bi(1,3,2-dioxaborolane) (546 mg, 2.15 mmol), and 1,4-dioxane (8 mL). The mixture was sparged with Ar for 5 minutes and then treated with Xphos-Pd-G2 (84 mg, 0.11 mmol). The mixture was sparged with Ar for another 5 minutes and then heated at 100° C. via microwave irradiation for 1 hour. The mixture was cooled to room temperature and then concentrated to dryness under reduced pressure. The product was purified by silica gel chromatography (eluent: petroleum ether:ethyl acetate=1:0 to 1:2) to afford the title compound (350 mg, 74%) as a brown solid. LC-MS (ESI), mass calcd. For C₁₉H₂₁BN₂O₄ 352.16 m/z found 353.1 [M+H]⁺.

Step C. 3-Hydroxy-3-(3-(2-(1-(phenylsulfonyl)-1H-pyrrolo[2,3-b]pyridin-3-yl)thiazol-4-yl)phenyl)-1H-pyrrolo[3,2-b]pyridin-2(3H)-one. To a microwave vial containing 4-bromo-2-(1-(phenylsulfonyl)-1H-pyrrolo[2,3-b]pyridin-3-yl)thiazole (Intermediate 2, 305 mg, 0.726 mmol), 3-hydroxy-3-(3-(4,4,5,5-tetramethyl-1,3,2-dioxaborolan-2-yl)phenyl)-1H-pyrrolo[3,2-b]pyridin-2(3H)-one (320 mg, 0.727 mmol) and K₃PO₄ (461 mg, 2.17 mmol) was added 1,4-dioxane (6 mL), and H₂O (1.5 mL). The mixture was sparged with Ar for 5 minutes and then treated with Pd(dtbpf)Cl₂ (49 mg, 0.075 mmol). The mixture was sparged with Ar for another 5 minutes and then heated at 80° C. via microwave irradiation for 1 hour. The mixture was cooled to room temperature and the solvent was removed under reduced pressure to give the product, which was purified by silica gel chromatography (eluent: petroleum ether:ethyl acetate=10:1 to 0:1) to afford the title compound (200 mg, 38%) as a brown solid. LC-MS (ESI): mass calcd. For C₂₉H₁₉N₅O₄S₂ 565.09 m/z found 566.1 [M+H]⁺.

### Intermediate 154. (R)-3-(3-chlorophenyl)-3-hydroxy-1-methyl-1,3-dihydro-2H-pyrrolo[3,2-b]pyridin-2-one

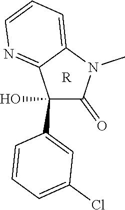

Step A. 1-Methyl-1H-pyrrolo[3,2-b]pyridine-2,3-dione. Sodium hydride (144 mg, 60 wt. % in mineral oil, 3.60 mmol) was added to a 0° C. suspension of 1H-pyrrolo[3,2-b]pyridine-2,3-done (500 mg, 3.27 mmol) in THE (15 mL). The resultant mixture was stirred at 0° C. for 30 minutes, and then treated with Mel (1.95 g, 13.7 mmol), and stirred at 0° C. for 2 hours. After this time, the mixture was quenched with sat. NH₄Cl (3 mL) and concentrated to dryness under reduced pressure to give the product, which was purified by silica gel chromatography (eluent: petroleum ether ethyl acetate=1:0 to 0:1) to afford the title compound (260 mg, 49%) as a yellow solid. ¹H NMR (400 MHz, DMSO-d₆) δ 8.35 (d, J=4.5 Hz, 1H), 7.63-7.53 (m, 2H), 3.13 (s, 3H).

Step 8. (R)-3-(3-Chlorophenyl)-3-hydroxy-1-methyl-1H-pyrrolo[3,2-b]pyridin-2(3H)-one. (3-Chlorophenyl)magnesium bromide (6.4 mL, 0.50 M in THF, 3.2 mmol) was added to a solution of 1-methyl-1H-pyrrolo[3,2-b]pyridine-2,3-dione (260 mg, 1.60 mmol) and THF (10 mL). The resultant mixture was stirred at room temperature for 12 hours. After this time, the reaction mixture was quenched with sat. NH₄Cl (10 mL) and concentrated to dryness under reduced pressure to give the product, which was initially purified by silica gel chromatography (eluent: petroleum ether:ethyl acetate=1:0 to 0:1). The product was further purified by preparative HPLC using an Xtimate C18, 150×40 mm×5 μm column (eluent: 25% to 55% (v/v) CH₃CN and H₂O with 0.05% NH₃H₂O) to afford the racemic product as a white solid. The (R) and (S) enantiomers of 3-(3-chlorophenyl)-3-hydroxy-1-methyl-1,3-dihydro-2H-pyrrolo[3,2-b]pyridin-2-one were separated by SFC over a DAICEL CHIRALPAK AD-H 250 mm×30 mm×5 μm column. An isocratic elution was used throughout the purification which consisted of EtOH: supercritical CO₂=30%: 70%. The first eluting enantiomer, (R)-3-(3-chlorophenyl)-3-hydroxy-1-methyl-1,3-dihydro-2H-pyrrolo[3,2-b]pyridin-2-one, (60 mg, 37%) was obtained as a white solid. LCMS (ESI): mass calcd. for C₁₄H₁₁ClN₂O₂ 274.05 m/z, found 275.1 [M+H]⁺. The second eluting enantiomer was (S)-3-(3-chlorophenyl)-3-hydroxy-1-methyl-1,3-dihydro-2H-pyrrolo[3,2-b]pyridin-2-one (Intermediate 155).

### Intermediate 155. (S)-3-(3-Chlorophenyl)-3-hydroxy-1-methyl-1,3-dihydro-2H-pyrrolo[3,2-b]pyridin-2-one

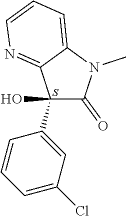

As described in the preparation of Intermediate 154, (S)-3-(3-chlorophenyl)-3-hydroxy-1-methyl-1,3-dihydro-2H-pyrrolo[3,2-b]pyridin-2-one, the second eluting enantiomer (60 mg, 37%) was obtained as a white solid. LCMS (ESI): mass calcd. for C₁₄H₁₁ClN₂O₂ 274.05 m/z, found 275.1 [M+H]⁺.

### Intermediate 156. (R)-3-hydroxy-1-methyl-3-(3-(2-(1-(phenylsulfonyl)-1H-pyrrolo[2,3-b]pyridin-3-yl)thiazol-4-yl)phenyl)-1,3-dihydro-2H-pyrrolo[3,2-b]pyridin-2-one

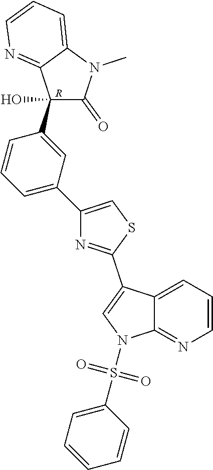

Step A. (R)-3-Hydroxy-1-methyl-3-(3-(4,4,5,5-tetramethyl-1,3,2-dioxaborolan-2-yl)phenyl)-1H-pyrrolo[3,2-b]pyridin-2(3H)-one. Potassium acetate (54 mg, 0.55 mmol) was added to a mixture of (R)-3-(3-chlorophenyl)-3-hydroxy-1-methyl-1H-pyrrolo[3,2-b]pyridin-2(3H)-one (Intermediate 154, 50 mg, 0.18 mmol), 4,4,4′,4′,5,5,5′,5′-octamethyl-2,2′-bi(1,3,2-dioxaborolane) (92 mg, 0.36 mmol), and 1,4-dioxane (3 mL). The mixture was sparged with Ar for 5 minutes and then treated with XPhos-Pd-G2 (14 mg, 0.018 mmol). The mixture was sparged with Ar for another 5 minutes and then heated at 85° C. via microwave irradiation for 1 hour. After this time, the mixture was cooled to room temperature and concentrated to dryness under reduced pressure to afford the product (70 mg, 58% purity, 61%) as a yellow solid. LC-MS (ESI): mass calcd. For C₂₀H₂₃BN₂O₄ 366.18 m/z found 367.3 [M+H]⁺.

Step B. (R)-3-Hydroxy-1-methyl-3-(3-(2-(1-tosyl-1H-pyrrolo[2,3-b]pyridin-3-yl)thiazol-4-yl)phenyl)-1H-pyrrolo[3,2-b]pyridin-2(3H)-one. To a microwave vial containing (R)-3-Hydroxy-1-methyl-3-(3-(4,4,5,5-tetramethyl-1,3,2-dioxaborolan-2-yl)phenyl)-1H-pyrrolo[3,2-b]pyridin-2(3H)-one (70 mg, 0.19 mmol), 4-bromo-2-(1-(phenylsulfonyl)-1H-pyrrolo[2,3-b]pyridin-3-yl)thiazole (Intermediate 2, 88 mg, 0.21 mmol) and K₃PO₄ (122 mg, 0.575 mmol) was added 1,4-dioxane (4 mL), and H₂O (1 mL). The resultant mixture was sparged with Ar for 5 minutes and then treated with Pd(dtbpf)Cl₂ (12 mg 0.018 mmol). The resultant mixture was heated at 85° C. via microwave irradiation for 1 hour. After this time, the mixture was cooled to room temperature, poured into water (10 mL) and extracted with methylene chloride (50 mL×3). The combined organic extracts were washed with brine (5 mL), dried over anhydrous Na₂SO₄, filtered, and concentrated to dryness under reduced pressure to give the product, which was purified by silica gel chromatography (eluent: petroleum ether:ethyl acetate=20:1 to 0:1) to afford the title compound (70 mg, 63%) as a white solid. LC-MS (ESI): mass calcd. For C₃₀H₂₁N₅O₄S₂ 579.10 m/z found 580.1 [M+H]⁺.

### Intermediate 157. (S)-3-Hydroxy-1-methyl-3-(3-(2-(1-(phenylsulfonyl)-1H-pyrrolo[2,3-b]pyridin-3-yl)thiazol-4-yl)phenyl)-1,3-dihydro-2H-pyrrolo[3,2-b]pyridin-2-one

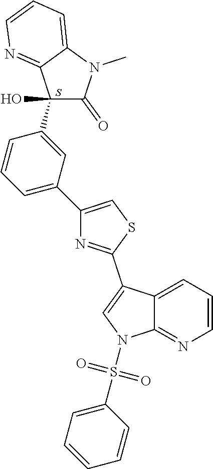

Step A. (S)-3-Hydroxy-1-methyl-3-(3-(4,4,5,5-tetramethyl-1,3,2-dioxaborolan-2-yl)phenyl)-1H-pyrrolo[3,2-b]pyridin-2(3H)-one. Potassium acetate (54 mg, 0.55 mmol) was added to a mixture of (S)-3-(3-chlorophenyl)-3-hydroxy-1-methyl-1H-pyrrolo[3,2-b]pyridin-2(3H)-one (50 mg, 0.18 mmol), 4,4,4′,4′,5,5,5′,5′-octamethyl-2,2′-bi(1,3,2-dioxaborolane) (92 mg, 0.36 mmol) and 1,4-dioxane (5 mL). The mixture was sparged with Ar for 5 minutes and then treated with XPhos-Pd-G2 (14 mg, 0.018 mmol). The mixture was sparged with Ar for another 5 minutes and then heated at 85° C. via microwave irradiation for 1 hour. After this time, the mixture was cooled to room temperature and concentrated to dryness under reduced pressure to afford the title compound (80 mg). LC-MS (ESI): mass calcd. For C₂₀H₂₃BN₂O₄ 366.18 m/z found 367.2 [M+H]⁺.

Step B. (S)-3-Hydroxy-1-methyl-3-(3-(2-(1-tosyl-1H-pyrrolo[2,3-b]pyridin-3-yl)thiazol-4-yl)phenyl)-1H-pyrrolo[3,2-b]pyridin-2(3H)-one. To a microwave vial containing (S)-3-hydroxy-1-methyl-3-(3-(4,4,5,5-tetramethyl-1,3,2-dioxaborolan-2-yl)phenyl)-1H-pyrrolo[3,2-b]pyridin-2(3H)-one (80 mg, 0.22 mmol), 4-bromo-2-(1-(phenylsulfonyl)-1H-pyrrolo[2,3-b]pyridin-3-yl)thiazole (Intermediate 2, 92 mg, 0.22 mmol) and K₃PO₄ (139 mg, 0.655 mmol) was added 1,4-dioxane (5 mL), and H₂O (1 mL). The resultant mixture was sparged with Ar for 5 minutes and then treated with Pd(dtbpf)Cl₂ (14 mg 0.021 mmol). The resultant mixture was heated at 85° C. via microwave irradiation for 1 hour. After this time, the mixture was cooled to room temperature, poured into water (10 mL), and extracted with methylene chloride (50 mL×3). The combined organic extracts were washed with brine (5 mL), dried over anhydrous Na₂SO₄, filtered, and concentrated to dryness under reduced pressure to give the product, which was purified by silica gel chromatography (eluent: petroleum ether:ethyl acetate=20:1 to 0:1) to afford the title compound (80 mg, 62%) as a white solid. LC-MS (ESI): mass calcd. For C₃₀H₂₁N₅O₄S₂ 579.10 m/z found 580.1 [M+H]⁺.

### Intermediate 158. (R)-3-Hydroxy-1-methyl-3-(3-(5-(1-tosyl-1H-pyrrolo[2,3-b]pyridin-3-yl)furan-3-yl)phenyl)pyrrolidin-2-one

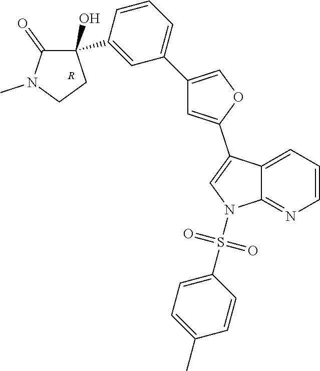

Step A. 3-lodo-1-tosyl-1H-pyrrolo[2,3-b]pyridine. 4-Toluenesulfonyl chloride (656 mg, 3.44 mmol) was added to a solution of 3-iodo-1H-pyrrolo[2,3-b]pyridine (700 mg, 2.87 mmol), DMAP (70 mg, 0.57 mmol), Et₃N (2.0 ml, 14 mmol), and methylene chloride (20 mL). The resultant mixture was stirred at room temperature for 12 hours. After this time, the reaction mixture was concentrated to dryness under reduced pressure to give the product, which was purified by silica gel chromatography (eluent: petroleum ether:ethyl acetate=20:1 to 5:1) to afford the title compound (1.1 g, 96%) as a white solid. LC-MS (ESI): mass calcd. For C₁₄H₁₁IN₂O₂S 397.96 m/z found 399.0 [M+H]⁺. 1H NMR (400 MHz, CDCl₃) δ 8.47 (dd, J=1.3, 4.8 Hz, 1H), 8.12 (d, J=8.3 Hz, 2H), 7.90 (s, 1H), 7.70 (dd, J=1.5, 7.8 Hz, 1H), 7.33 (s, 1H), 7.31-7.28 (m, 2H), 2.41 (s, 3H).

Step B. 3-(4-Bromofuran-2-yl)-1-tosyl-1H-pyrrolo[2,3-b]pyridine. To a flask containing 3-iodo-1-tosyl-1H-pyrrolo[2,3-b]pyridine (400 mg, 1.00 mmol), 2-(4-bromofuran-2-yl)-4,4,5,5-tetramethyl-1,3,2-dioxaborolane (219 mg, 0.802 mmol) and K₃PO₄ (640 mg, 3.02 mmol) was added 1,4-dioxane (20 mL), and H₂O (2 mL). The mixture was sparged with N₂ for 5 minutes and then treated with Pd(PPh₃)₄ (116 mg, 0.100 mmol). The resultant mixture was heated at 70° C. for 16 hours. After this time, the mixture was cooled to room temperature. The suspension was filtered through a pad of diatomaceous earth and the filter cake was washed with water (10 mL). The filtrate was extracted with methylene chloride (30 mL×3). The combined organic extracts were washed with brine (10 mL), dried over anhydrous Na₂SO₄, filtered, and concentrated to dryness under reduced pressure to give the product, which was purified by silica gel chromatography (eluent: petroleum ether:ethyl acetate=20:1 to 5:1) to afford the title compound (190 mg, 68% purity, 31%) as a yellow solid. LC-MS (ESI): mass calcd. For C₁₈H₁₃BrN₂O₃S 415.98 m/z found 417.2 [M+H]⁺.

Step C. (R)-3-Hydroxy-1-methyl-3-(3-(5-(1-tosyl-1H-pyrrolo[2,3-b]pyridin-3-yl)furan-3-yl)phenyl)pyrrolidin-2-one. To a microwave vial containing 3-(4-bromofuran-2-yl)-1-tosyl-1H-pyrrolo[2,3-b]pyridine (150 mg, 0.244 mmol), (R)-3-hydroxy-1-methyl-3-(3-(4,4,5,5-tetramethyl-1,3,2-dioxaborolan-2-yl)phenyl)pyrrolidin-2-one (Intermediate 16, 78 mg, 0.25 mmol) and sat. Na₂CO₃ (0.3 mL) was added toluene (8 mL) and EtOH (2 mL). The mixture was sparged with N₂ for 5 minutes and then treated with Pd(PPh₃)₄ (51 mg, 0.044 mmol). The mixture was sparged with Ar for another 5 minutes and then heated at 80° C. via microwave irradiation for 1 hour. After this time, the mixture was cooled to room temperature. The suspension was filtered through a pad of diatomaceous earth and the pad was washed with ethyl acetate (20 mL). The filtrate was concentrated to dryness under reduced pressure to afford the product, which was purified by silica gel chromatography (eluent: petroleum ether:ethyl acetate=1:0 to 0:1) to afford the title compound (90 mg, 65%) as a yellow solid. LC-MS (ESI): mass calcd. For C₂₉H₂₅N₃O₅S 527.15 m/z found 528.2 [M+H]⁺.

### Intermediate 159. (R)-3-(3-(2-(4,5-Difluoro-1-tosyl-1H-pyrrolo[2,3-b]pyridin-3-yl)thiazol-4-yl)phenyl)-3-hydroxy-1-methylpyrrolidin-2-one

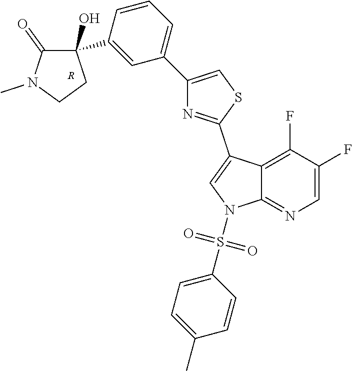

Step A. 4,5-Difluoro-1-tosyl-1H-pyrrolo[2,3-b]pyridine. TosCl (1.19 g, 6.24 mmol) was added to a solution of 4,5-difluoro-1H-pyrrolo[2,3-b]pyridine (800 mg, 5.19 mmol), DMAP (127 mg, 1.04 mmol), Et₃N (3.60 mL, 25.9 mmol), and dichloromethane (40 mL). The resultant mixture was stirred at room temperature for 16 hours before quenching with H₂O (100 mL) and extracting with dichloromethane (50 mL×3). The combined organic extracts were washed with brine (50 mL), dried over anhydrous Na₂SO₄, filtered, and concentrated to dryness under reduced pressure to give the product, which was purified by silica gel chromatography (eluent: petroleum ether:ethyl acetate=1:0 to 5:1) to afford the title compound (1.48 g, 90%) as a white solid. LC-MS (ESI): mass calcd. For C₁₄H₁₀F₂N₂O₂S 308.04 m/z, found 309.0 [M+H]⁺.

Step B. 3-Bromo-4,5-difluoro-1-tosyl-1H-pyrrolo[2,3-b]pyridine. NBS (1.03 g, 5.79 mmol) was added to a solution of 4,5-difluoro-1-tosyl-1H-pyrrolo[2,3-b]pyridine (1.48 g, 4.80 mmol) and DMF (20 mL). The reaction mixture was heated at 50° C. for 1.5 hours. The mixture was cooled to room temperature and was concentrated to dryness under reduced pressure to give the product, which was purified by silica gel chromatography (eluent: petroleum ether:ethyl acetate=10:1 to 4:1) to afford the compound (1.66 g, 84%) as a white solid. LC-MS (ESI): mass calcd. For C₁₄H₉BrF₂N₂O₂S 385.95 m/z, found 388.6 [M+H]⁺.

Step C. 4,5-Difluoro-1-tosyl-3-(trimethylstannyl)-1H-pyrrolo[2,3-b]pyridine. To a flask containing 3-bromo-4,5-difluoro-1-tosyl-1H-pyrrolo[2,3-b]pyridine (650 mg, 1.68 mmol) was added toluene (15 mL) and 1,1,1,2,2,2-hexamethyldistannane (1.46 g, 4.46 mmol). The mixture was sparged with N₂ for 5 minutes and then treated with Pd(PPh₃)₄ (360 mg, 0.312 mmol). The mixture was sparged with N₂ for another 5 minutes and then heated at 115° C. for 2 hours. After this time, the mixture was cooled to room temperature, and quenched with KF (150 mL) and stirred for 2 hours. The aqueous mixture was extracted with ethyl acetate (80 mL×3). The combined organic extracts were dried over anhydrous Na₂SO₄, filtered, and concentrated to dryness under reduced pressure to give the product, which was purified by silica gel chromatography (eluent: petroleum ether:ethyl acetate=1:0 to 5:1) to afford the title compound (730 mg, 72%) as a colorless oil. LC-MS (ESI): mass calcd. For C₁₇H₁₈F₂N₂O₂SSn 472.01 m/z, found 473.1 [M+H]⁺.

Step D. 4-Bromo-2-(4,5-difluoro-1-tosyl-1H-pyrrolo[2,3-b]pyridin-3-yl)thiazole. 4,5-Difluoro-3-(4,4,5,5-tetramethyl-1,3,2-dioxaborolan-2-yl)-1-tosyl-1H-pyrrolo[2,3-b]pyridine (730 mg, 1.55 mmol), 2,4-dibromothiazole (753 mg, 3.10 mmol), and 1,4 dioxane (10 mL) were added to a 100 mL three-necked round-bottomed flask. The mixture was sparged with N₂ for 5 minutes and then treated with Pd(PPh₃)₄ (179 mg, 0.155 mmol). The mixture was sparged with N₂ for another 5 minutes and then heated at 120° C. for 16 hours. After this time, the contents were cooled to room temperature and the solids were removed via filtration. The filtrate was concentrated to dryness under reduced pressure to give the product, which was purified by silica gel chromatography (eluent: petroleum ether ethyl acetate=1:0 to 5:1) to afford the title compound (700 mg) as a white solid. LC-MS (ESI): mass calcd. For C₁₇H₁₀BrF₂N₃O₂S₂ 468.94 m/z, found 470.0 [M+H]⁺. ¹H NMR (400 MHz, CDCl₃-d) S 8.46 (s, 1H), 8.44-8.38 (m, 1H), 8.15-8.05 (m, 2H), 7.32 (d, J=8.2 Hz, 2H), 7.27 (d, J=6.6 Hz, 1H), 2.40 (s, 3H).

Step E. (R)-3-(3-(2-(4,5-Difluoro-1-tosyl-1H-pyrrolo[2,3-b]pyridin-3-yl)thiazol-4-yl)phenyl)-3-hydroxy-1-methylpyrrolidin-2-one. (R)-3-Hydroxy-1-methyl-3-(3-(4,4,5,5-tetramethyl-1,3,2-dioxaborolan-2-yl)phenyl)pyrrolidin-2-one (Intermediate 16, 337 mg, 1.06 mmol), 4-bromo-2-(4,5-difluoro-1-tosyl-1H-pyrrolo[2,3-b]pyridin-3-yl)thiazole (500 mg, 1.06 mmol), K₃PO₄ (677 mg, 3.19 mmol), H₂O (5 mL), and 1,4-dioxane (20 mL) were added to a 100 mL three-necked round-bottomed flask. The resultant mixture was sparged with N₂ for 5 minutes and then treated with Pd(dtbpf)Cl₂ (69 mg, 0.11 mmol). The resultant mixture was heated at 90° C. for 16 hours. After this time, the contents were cooled to room temperature, and the solids were removed via filtration. The filtrate was extracted with ethyl acetate (70 mL×3). The combined organic extracts were washed with brine, dried over anhydrous Na₂SO₄, filtered, and concentrated to dryness under reduced pressure to give the product, which was purified by silica gel chromatography (eluent: petroleum ether ethyl acetate=1:0 to 0:1) to afford the title compound (270 mg, 39%). LC-MS (ESI): mass calcd. For C₂₈H₂₂F₂N₄O₄S₂ 580.11 m/z, found 581.2 [M+H]⁺.

### Intermediate 160. 4-Bromo-2-(5-chloro-4-fluoro-1H-pyrrolo[2,3-b]pyridin-3-yl)thiazole

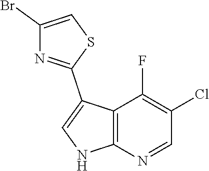 [SMILES: Fc1c(Cl)cnc2[nH]cc(-c3nc(Br)cs3)c12]

Step A. 5-Chloro-4 fluoro-1-(triisopropylsilyl)-1H-pyrrolo[2,3-b]pyridine. sec-Butyllithium (12.0 mL, 1.3 M in cyclohexane, 15.6 mmol) was added dropwise to a −78° C. (dry ice/ethanol) solution of 4-fluoro-1-(triisopropylsilyl)-1H-pyrrolo[2,3-b]pyridine (2.00 g, 6.84 mmol) and THF (15 ml). The mixture was stirred for 0.5 h at −78° C. Then a solution of perchloroethane (3.95 g, 16.7 mmol) and THF (45 ml) was added rapidly. The reaction mixture was stirred at −78° C. for another 1 hour and then quenched with sat. NH₄Cl (60 mL). The aqueous portion was extracted with ethyl acetate (60 mL×4). The combined organic extracts were dried with anhydrous Na₂SO₄, filtered and concentrated to dryness under reduced pressure to give the product, which was purified by silica gel chromatography (eluent: petroleum ether:ethyl acetate=1:0 to 0:1) to afford the title compound (1.92 g, 78%) as a white solid. LC-MS (ESI): mass calcd. for C₁₆H₂₄ClFN₂Si 326.14 m/z found 327.2 [M+H]⁺.

Step B. 3-Bromo-5-chloro-4-fluoro-1-(triisopropylsilyl)-1H-pyrrolo[2,3-b]pyridine. NBS (1.17 g, 6.57 mmol) was added to a solution of 5-chloro-4-fluoro-1-(triisopropylsilyl)-1H-pyrrolo[2,3-b]pyridine (1.92 g, 5.87 mmol) in chloroform (60 ml). The reaction mixture was stirred at room temperature for 16 hours before pouring it into water (60 mL) and extracting with dichloromethane (60 mL×4). The combined organic extracts were dried with anhydrous Na₂SO₄, filtered and concentrated to dryness under reduced pressure to give the product, which was purified by silica gel chromatography (eluent: petroleum ether:ethyl acetate=1:0 to 0:1) to afford the title compound (1.70 g, 69%) as a white solid. LC-MS (ESI): mass calcd. for C₁₆H₂₃BrClFN₂Si 404.05 m/z found 405.1 [M+H]⁺.

Step C. 5-Chloro-4 fluoro-3-(4,4,5,5-tetramethyl-1,3,2-dioxaborolan-2-yl)-1-(triisopropylsilyl)-1H-pyrrolo[2,3-b]pyridine. KOAc (406 mg, 4.14 mmol) was added to a solution of 3-bromo-5-chloro-4-fluoro-1-(triisopropylsilyl)-1H-pyrrolo[2,3-b]pyridine (559 mg, 1.38 mmol), 4,4,4′,4′,5,5,5′,5′-octamethyl-2,2′-bi(1,3,2-dioxaborolane) (700 mg, 2.76 mmol) in 1,4-dioxane (50 mL). The resultant mixture was purged with N₂ for 5 min and then treated with Pd(dppf)Cl₂ (101 mg, 0.14 mmol). The resultant mixture was again purged with N₂ for 5 min and then heated at 100° C. for 2 hours. The mixture was cooled to room temperature, filtered through a pad of diatomaceous earth, and the pad was washed with ethyl acetate (20 mL). The filtrate was concentrated to dryness under reduced pressure to afford the product (1.3 g) as a brown solid, which was used in the next step without further purification. LC-MS (ESI): mass calcd. for C₂₂H₃₅BClFN₂O₂Si 452.22 m/z found 453.2 [M+H]⁺.

Step D. 4-Bromo-2-(5-chloro-4-fluoro-1H-pyrrolo[2,3-b]pyridin-3-yl)thiazole. 5-Chloro-4 fluoro-3-(4,4,5,5-tetramethyl-1,3,2-dioxaborolan-2-yl)-1-(triisopropylsilyl)-1H pyrrolo[2,3-b]pyridine (1000 mg), 2,4-dibromothiazole (805 mg, 3.31 mmol), and Cs₂CO₃ (1.439 g, 4.416 mmol) were added to a 250 mL round-bottomed flask and dissolved in 1,4-dioxane (40 mL) and H₂O (10 mL). The resultant mixture was purged with Ar for 5 minutes and then treated with Pd(dppf)Cl₂ (162 mg, 0.221 mmol). The resultant mixture was purged with Ar for another 5 minutes and then heated at 95° C. for 4 hours. After this time, the reaction mixture was cooled to room temperature, diluted with H₂O (50 mL), and extracted with ethyl acetate (50 mL×4). The combined organic extracts were washed with brine (20 mL), dried over anhydrous Na₂SO₄, filtered, and concentrated to dryness under reduced pressure. The product was initially purified by silica gel chromatography (eluent: petroleum ether:ethyl acetate=1:0 to 0:1, then ethyl acetate:methanol=10:1). The product was further purified by preparative HPLC using a Boston Prime C18 150×40 mm×5 μm column (eluent: 49% to 79% (v/v) CH₃CN and H₂O with 0.05% HCl) to afford the title compound (70 mg, 9%) as a white solid. LC-MS (ESI): mass calcd. for C₁₀H₄BrClFN₃S 330.90 m/z found 331.9 [M+H]⁺. ¹H NMR (400 MHz, DMSO-d₆) δ 12.99 (br s, 1H), 8.46 (d, J=9.0 Hz, 1H), 8.35 (d, J=2.9 Hz, 1H), 7.78 (5, 1H).

### Intermediate 161. (R)-7-(3-(2-(4,5-Difluoro-1-tosyl-1H-pyrrolo[2,3-b]pyridin-3-yl)thiazol-4-yl)phenyl)-6,7-dihydro-5H-pyrrolo[1,2-a]imidazol-7-ol

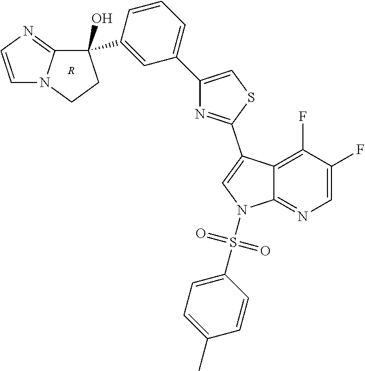

To a microwave vial containing (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13, 240 mg, 0.736 mmol), 4-bromo-2-(4,5-difluoro-1-tosyl-1H-pyrrolo[2,3-b]pyridin-3-yl)thiazole (415 mg, 0.882 mmol) and K₃PO₄ (469 mg, 2.21 mmol) was added H₂O (2 mL), and 1,4-dioxane (8 mL). The resultant mixture was sparged with N₂ for 5 minutes and then treated with Pd(dtbpf)Cl₂ (48 mg, 0.074 mmol). The mixture was heated at 90° C. via microwave irradiation for 1 hour and then allowed to cool to room temperature. The reaction mixture was concentrated to dryness under reduced pressure to give the product, which was purified by silica gel chromatography (eluent: petroleum ether ethyl acetate=1:0 to 0:1) to afford the title compound (300 mg, 47%). LC-MS (ESI): mass calcd. For C₂₉H₂₁F₂N₅O₃S₂ 589.11 m/z, found 590.2 [M+H]⁺.

### Intermediate 162. (R)-4-(3-(3-Hydroxy-1-methyl-2-oxopyrrolidin-3-yl)phenyl)-2-(1-tosyl-1H-pyrrolo[2,3-b]pyridin-3-yl)thiazole-5-carbonitrile

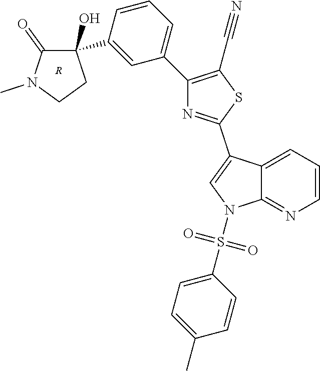

Step A. 4-Chloro-2-(1-tosyl-1H-pyrrolo[2,3-b]pyridin-3-yl)thiazole-5-carbonitrile. Xphos-Pd-G2 (178 mg, 0.226 mmol) was added to a mixture of 3-(4,4,5,5-tetramethyl-1,3,2-dioxaborolan-2-yl)-1-tosyl-1H-pyrrolo[2,3-b]pyridine (900 mg, 2.26 mmol), 2,4-dichlorothiazole-5-carbonitrile (485 mg, 2.71 mmol), KPO₄ (1.44 g, 6.78 mmol) and 1,4-dioxane/H₂O=4:1 (12 mL) under N₂ atmosphere. The mixture was heated for 1 hour at 100° C. After this time, the mixture was poured into water (30 mL). The aqueous layer was extracted with ethyl acetate (30 mL×3). The combined organic layers were washed with brine (50 mL), dried over Na₂SO₄, filtered, and concentrated to dryness under reduced pressure to give the product, which was purified by silica gel chromatography (petroleum ether:ethyl acetate=1:0 to 1:1) to afford the title compound (500 mg, 48%) as a white solid. LCMS (ESI): mass calcd. for C₁₈H₁₁ClN₄O₂S₂ 414.00 m/z, found 414.9 [M+H]⁺.

Step B. (R)-4-(3-(3-hydroxy-1-methyl-2-oxopyrrolidin-3-yl)phenyl)-2-(1-tosyl-1H-pyrrolo[2,3-b]pyridin-3-yl)thiazole-5-carbonitrile. Xphos-Pd-G2 (95 mg, 0.12 mmol) was added to a mixture of 4-chloro-2-(1-tosyl-1H-pyrrolo[2,3-b]pyridin-3-yl)thiazole-5-carbonitrile (500 mg, 1.21 mmol), (R)-3-hydroxy-1-methyl-3-(3-(4,4,5,5-tetramethyl-1,3,2-dioxaborolan-2-yl)phenyl)pyrrolidin-2-one (Intermediate 16, 459 mg, 1.45 mmol), KPO₄ (767 mg, 3.62 mmol), and 1,4-dioxane/H₂O (4/1, 12 mL) under N₂ atmosphere. The mixture was heated for 1 hour at 100° C. via microwave irradiation. After this time, the mixture was poured into water (10 mL) and extracted with ethyl acetate (10 mL×3). The combined organic layers were washed with brine (20 mL), dried over Na₂SO₄, filtered, and concentrated to dryness under reduced pressure to afford the title compound (680 mg, 53%) as a yellow solid. LCMS (ESI): mass calcd. For C₂₉H₂₃N₅O₄S₂ 569.12 m/z, found 570.1 [M+H]⁺.

### Intermediate 163. 3-(3-(5-Fluoro-2-(1-tosyl-1H-pyrrolo[2,3-b]pyridin-3-yl)thiazol-4-yl)phenyl)-1-methyl-2-oxopyrrolidin-3-yl acetate

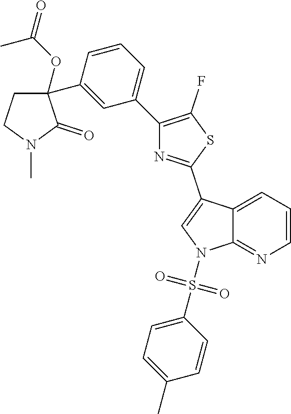 [SMILES: CC(=O)OC1(c2cccc(-c3nc(-c4cn(S(=O)(=O)c5ccc(C)cc5)c5ncccc45)sc3F)c2)CCN(C)C1=O]

Step A. 2-(5-Fluorothiazol-2-yl)isoindoline-1,3-dione. Phthalic acid anhydride (13.3 g, 89.8 mmol) was added to a solution of 5-fluorothiazol-2-amine hydrochloride (7.0 g, 45 mmol), triethylamine (9.4 mL, 68 mmol), and 1,4-dioxane (120 mL). The mixture was heated at 110° C. for 16 hours. After this time, the mixture was diluted with methylene chloride (20 mL) and filtered through a pad of diatomaceous earth. The filtrate was concentrated to dryness to afford the product, which was purified by silica gel chromatography (eluent: petroleum ether:methylene chloride=1:0 to 0:1) to give the title compound (7.0 g, 62%) as a yellow solid. LC-MS (ESI): mass calcd. for C₁₁H₅FN₂O₂S 248.01 m/z found 249.0 [M+H]⁺.

Step B. 2-(4-Bromo-5-fluorothiazol-2-yl)isoindoline-1,3-dione. Azobisisobutyronitrile (529 mg, 3.22 mmol) was added to a solution of 1,3-dibromo-5,5-dimethylimidazolidine-2,4-dione (5.53 g, 19.3 mmol), 2-(5-fluorothiazol-2-yl)isoindoline-1,3-dione (4.0 g, 16 mmol), and CCl₄ (50 mL). The resultant mixture was stirred at 75° C. for 16 hours. After this time, the mixture was cooled to room temperature, quenched with sat. Na₂SO₃ (30 mL) and extracted with methylene chloride (40 mL×3). The combined organic extracts were concentrated to dryness to afford the product, which was purified by silica gel chromatography (eluent: petroleum ether:ethyl acetate=1:0 to 1:1) to give the title compound (4 g, 76%) as a yellow solid. LC-MS (ESI): mass calcd. for C₁₁H₄BrFN₂O₂S 325.92 m/z found 326.7 [M+H]⁺.

Step C. 2-((5-Fluoro-4-(3-(3-hydroxy-1-methyl-2-oxopyrrolidin-3-yl)phenyl)thiazol-2-yl)carbamoyl)benzoic acid. 2-(4-Bromo-5-fluorothiazol-2-yl)isoindoline-1,3-dione (1.0 g, 3.06 mmol), (R,S)-3-hydroxy-1-methyl-3-(3-(4,4,5,5-tetramethyl-1,3,2-dioxaborolan-2-yl)phenyl)pyrrolidin-2-one (Intermediate 4, 1.0 g, 1.58 mmol), and K₃PO₄ (1.0 g, 4.7 mmol) were dissolved in 1,4-dioxane (6 mL) and H₂O (1.5 mL). The mixture was sparged with Ar for 5 minutes and then treated with Xphos-Pd-G2 (600 mg, 0.763 mmol). The resultant mixture was sparged with Ar for another 5 minutes and then heated at 100° C. for 6 hours. After this time, the mixture was cooled to room temperature, diluted with water (100 mL) and extracted with ethyl acetate (100 mL×3). The aqueous phase was acidified by 1N HCl to pH=3 and extracted with CH₂Cl₂/MeOH (v/v=9:1, 100 mL×4). The combined organic extracts were concentrated to dryness to afford the title compound (1.2 g, 33%) as a brown oil. LC-MS (ESI): mass calcd. for C₂₂H₁₆FN₃O₅S 455.10 m/z found 456.0 [M+H]⁺.

Step D. 3-(3-(2-(1,3-Dioxoisoindolin-2-yl)-5-fluorothiazol-4-yl)phenyl)-1-methyl-2-oxopyrrolidin-3-yl acetate. Potassium acetate (220 mg, 2.68 mmol) was added to a solution of 2-((5-fluoro-4-(3-(3-hydroxy-1-methyl-2-oxopyrrolidin-3-yl)phenyl)thiazol-2-yl)carbamoyl)benzoic acid (1.2 g, 2.6 mmol) and Ac₂O (10 mL). The resultant mixture was heated at 100° C. for 4 hours. After this time, the mixture was washed with water (50 mL) and extracted with methylene chloride (30 mL×4). The combined organic extracts were washed with sat. NaHCO₃ (80 mL), dried over anhydrous Na₂SO₄, filtered, and concentrated to dryness to afford the product, which was purified by silica gel chromatography (eluent: petroleum ether:ethyl acetate=1:0 to 0:1) to give the title compound (520 mg, 32%) as a light brown solid. LC-MS (ESI): mass calcd. for C₂₄H₁₈FN₃O₅S 479.10 m/z found 502.0 [M+Na]⁺.

Step E. 3-(3-(2-Amino-5-fluorothiazol-4-yl)phenyl)-1-methyl-2-oxopyrrolidin-3-yl acetate. NH₂NH₂·H₂O (190 μL, 85 wt. %, 3.33 mmol) was added to a solution of 3-(3-(2 (1,3-dioxoisoindolin-2-yl)-5 fluorothiazol-4-yl)phenyl)-1-methyl-2-oxopyrrolidin-3-yl acetate (940 mg, 1.96 mmol) and MeOH (10 mL). The resultant mixture was heated at 30° C. for 2 hours. After this time, the mixture was concentrated under reduced pressure to afford the product, which was purified by silica gel chromatography (eluent: petroleum ether:ethyl acetate=1:0 to 0:1) to give the title compound (390 mg, 51%) as a yellow solid. LC-MS (ESI): mass calcd. for C₁₆H₁₆FN₃O₃S 349.09 m/z found 372.0 [M+Na]⁺.

Step F. 3-(3-(2-Bromo-5 fluorothiazol-4-yl)phenyl)-1-methyl-2-oxopyrrolidin-3-yl acetate. tert-Butyl nitrite (120 NL, 1.00 mmol) was added to a 0° C. solution of 3-(3-(2-amino-5 fluorothiazol-4-yl)phenyl)-1-methyl-2-oxopyrrolidin-3-yl acetate (240 mg, 0.687 mmol), CuBr₂ (185 mg, 0.828 mmol), and CH₃CN (5 mL). The resultant mixture was stirred for 1 hour with gradual warming to room temperature. After this time, the mixture was diluted with water (15 mL) and extracted with methylene chloride (15 mL×3). The combined organic extracts were dried over anhydrous Na₂SO₄, filtered, and concentrated to dryness to afford the product, which was purified by silica gel chromatography (eluent: petroleum ether:ethyl acetate=1:0 to 0:1) to give the title compound (50 mg, 15%) as a yellow oil. LC-MS (ESI): mass calcd. for C₁₆H₁₄BrFN₂O₃S 411.99 m/z, found 434.9 [M+Na]⁺.

Step G. 3-(3-(5-Fluoro-2-(1-tosyl-1H-pyrrolo[2,3-b]pyridin-3-yl)thiazol-4-yl)phenyl)-1-methyl-2-oxopyrrolidin-3-yl acetate. To a microwave vial containing 3-(3-(2-Bromo-5 fluorothiazol-4-yl)phenyl)-1-methyl-2-oxopyrrolidin-3-yl acetate (85 mg, 0.21 mmol), 3-(4,4,5,5-tetramethyl-1,3,2-dioxaborolan-2-yl)-1-tosyl-1H-pyrrolo[2,3-b]pyridine (100 mg, 0.251 mmol), and K₃PO₄ (130 mg, 0.612 mmol) were added 1,4-dioxane (1.6 mL) and H₂O (0.4 mL). The resultant mixture was sparged with Ar for 5 minutes and then treated with Pd(dtbpf)Cl₂ (15 mg, 0.023 mmol). The mixture was sparged with Ar for another 5 minutes and then heated at 85° C. via microwave irradiation for 1 hour. After this time, the mixture was cooled to room temperature, diluted with water (6 mL) and extracted with methylene chloride/MeOH (9/1, 8 mL×5). The combined organic extracts were dried over anhydrous Na₂SO₄, filtered, and concentrated under reduced pressure to afford the title compound (180 mg, 77%) as a dark solid. LC-MS (ESI): mass calcd. for C₃₀H₂₅FN₄O₅S₂ 604.13 m/z found 605.0 [M+H]⁺.

### Intermediate 164. (3R)-3-Hydroxy-1-methyl-3-(3-(2-(1-(tetrahydro-2H-pyran-2-yl)-1H-pyrazolo[3,4-b]pyridin-3-yl)thiazol-4-yl)phenyl)pyrrolidin-2-one

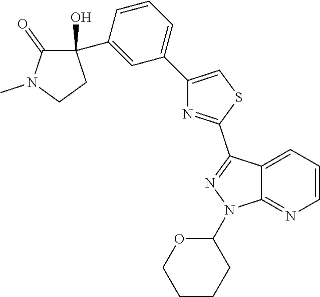 [SMILES: CN1CC[C@@](O)(c2cccc(-c3csc(-c4nn(C5CCCCO5)c5ncccc45)n3)c2)C1=O]

Step A. 2-Fluoro-N-methoxy-N-methylnicotinamide. TBTU (15.9 g, 49.6 mmol) was added to a solution of 2-fluoronicotinic acid (7.00 g, 49.6 mmol), N, O-dimethyl-hydroxylamine (5.32 g, 54.6 mmol), DIPEA (25.9 mL, 149 mmol) and DCM (100 mL). The reaction mixture was stirred for 16 hours at room temperature. The reaction mixture was quenched with H₂O (200 mL) and extracted with DCM (100 mL×3). The combined organic extracts were washed with brine (200 mL), dried over anhydrous Na₂SO₄, filtered, and concentrated to dryness. The residue was subjected to silica gel chromatography (0-30% EtOAc/pet ether) to give 2-fluoro-N-methoxy-N-methylnicotinamide as yellow oil (9.4 g, 86%). LC-MS (ESI): mass calcd. for C₈H₉FN₂O₂ 184.06 m/z, found 185.1 [M+H]⁺.

Step B. (4-Bromothiazol-2-yl)(2 fluoropyridin-3-yl)methanone. i-PrMgCl (16.1 mL, 2 M in THF, 32.1 mmol) was added to a 0° C. solution of 2,4-dibromothiazole (7.10 g, 29.2 mmol) and THF (60 mL) under N₂. The reaction mixture was stirred for 2 hours at 0° C., and then treated with a solution of 2-fluoro-N-methoxy-N-methylnicotinamide (5.92 g, 32.2 mmol) and THF (10 mL) at 0° C. The mixture was stirred for 16 hours at 40° C. After this time, the mixture was diluted with water (50 mL) and extracted with ethyl acetate (50 mL×3). The combined organic extracts were washed with brine (50 mL), dried over anhydrous Na₂SO₄, filtered, and concentrated to dryness in vacuo. The residue was subjected to silica gel chromatography (0-30% EtOAc/pet ether) to give (4-bromothiazol-2-yl)(2-fluoropyridin-3-yl)methanone as a yellow solid (5.1 g, 59%). LC-MS (ESI): mass calcd. for C₉H₄BrFN₂OS 285.92 m/z, found 286.9 [M+H]⁺.

Step C. 4-Bromo-2-(1H-pyrazolo[3,4-b]pyridin-3-yl)thiazole. N₂H₄·H₂O (4.32 mL, 88.8 mmol) was added to a solution of (4-bromothiazol-2-yl)(2-fluoropyridin-3-yl)methanone (5.10 g, 17.8 mmol) and THE (100 mL). The mixture was heated at 75° C. for 16 hours. After this time, the mixture was cooled to room temperature, diluted with water (100 mL) and extracted with ethyl acetate (50 mL×3). The combined organic extracts were washed with brine (50 mL), dried over anhydrous Na₂SO₄, filtered, and concentrated to dryness in vacuo. The residue was subjected to silica gel chromatography (0-30% EtOAc/pet ether) to give 4-bromo-2-(1H-pyrazolo[3,4-b]pyridin-3-yl)thiazole as a yellow solid (3.5 g, 64%). LC-MS (ESI): mass calcd. for C₉H₅BrN₄S 279.94 m/z, found 281.0 [M+H]⁺.

Step D. 4-Bromo-2-(1-(tetrahydro-2H-pyran-2-yl)-1H-pyrazolo[3,4-b]pyridin-3-yl)thiazole. A mixture of 4-bromo-2-(1H-pyrazolo[3,4-b]pyridin-3-yl)thiazole (500 mg, 1.78 mmole), 3,4-dihydro-2H-pyran (0.487 mL, 5.34 mmole), and 4-methylbenzenesulfonic acid (61 mg, 0.36 mmole) in THE (10 mL) was heated for 16 h at 70° C. After this time, the mixture was cooled to r.t., diluted with aq. NaHCO₃ (10 mL), and extracted with ethyl acetate (10 mL×3). The combined organic extracts were washed with brine (10 mL), dried over anhydrous Na₂SO₄, filtered, and concentrated to dryness in vacuo to give the product, which was subjected to silica gel chromatography (0-20% EtOAc/pet ether) to give 4-bromo-2-(1-(tetrahydro-2H-pyran-2-yl)-1H-pyrazolo[3,4-b]pyridin-3-yl)thiazole as a white solid (550 mg, 61%). LC-MS (ESI): mass calcd. for C₁₄H₁₃BrN₄OS 364.00 m/z, found 365.0 [M+H]⁺.

Step E. (3R)-3-Hydroxy-1-methyl-3-(3-(2-(1-(tetrahydro-2H-pyran-2-yl)-1H-pyrazolo[3,4-b]pyridin-3-yl)thiazol-4-yl)phenyl)pyrrolidin-2-one. To a microwave vial containing 4-bromo-2-(1-(tetrahydro-2H-pyran-2-yl)-1H-pyrazolo[3,4-b]pyridin-3-yl)thiazole (500 mg, 1.37 mmol), (R)-3-hydroxy-1-methyl-3-(3-(4,4,5,5-tetramethyl-1,3,2-dioxaborolan-2-yl)phenyl)pyrrolidin-2-one (Intermediate 16, 434 mg, 1.37 mmol), and Cs₂CO₃ (1.34 g, 4.11 mmol) were added 1,4-dioxane (8 mL) and H₂O (2 mL). The resultant mixture was sparged with Ar for 5 min, treated with XPhos Pd G₂ (108 mg, 0.137 mmol), and heated at 100° C. via microwave irradiation for 1 h. After this time, the mixture was cooled to r.t., and concentrated to dryness in vacuo. The residue was subjected to silica gel chromatography (0-100% EtOAc/pet ether) to give (3R)-3-hydroxy-1-methyl-3-(3-(2-(1-(tetrahydro-2H-pyran-2-yl)-1H-pyrazolo[3,4-b]pyridin-3-yl)thiazol-4-yl)phenyl)pyrrolidin-2-one as a white solid (188 mg, 24%). LC-MS (ESI): mass calcd. for C₂₅H₂₅N₅O₃S 475.17 m/z, found 476.2 [M+H]⁺.

### Intermediate 165. 7-Hydroxy-7-(3-(2-(1-(phenylsulfonyl)-1H-pyrrolo[2,3-b]pyridin-3-yl)thiazol-4-yl)phenyl)-6,7-dihydro-5H-cyclopenta[b]pyridin-5-yl acetate

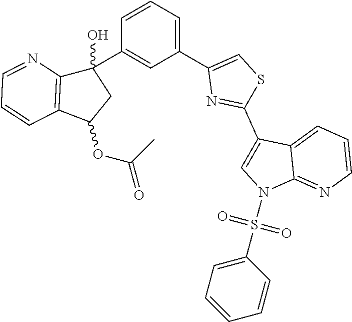 [SMILES: CC(=O)OC1CC(O)(c2cccc(-c3csc(-c4cn(S(=O)(=O)c5ccccc5)c5ncccc45)n3)c2)c2ncccc21]

Step A. 7-(3-Bromophenyl)-6,7-dihydro-5H-cyclopenta[b]pyridin-7-ol. n-Butyllithium (25.2 mL, 2.5 M in hexane, 63.1 mmol) was added dropwise to a −72° C. (dry ice/EtOH) solution of 1,3-dibromobenzene (16.1 g, 68.3 mmol) and anhydrous THF (100 mL). The resultant mixture was stirred at −72° C. (dry ice/EtOH) for 30 minutes, and then treated with a solution of 5H-cyclopenta[b]pyridin-7(6H)-one (7.00 g, 52.6 mmol) and anhydrous THF (150 mL) at −72° C. The mixture was stirred for 30 minutes at −72° C. After this time, the mixture was quenched with water (150 mL) and extracted with ethyl acetate (100 mL×3). The combined organic extracts were washed with brine (150 mL), dried over Na₂SO₄, filtered, and concentrated to dryness under reduced pressure to give a residue, which was purified by FCC (eluent: petroleum ether:ethyl acetate=1:0 to 1:1) to afford the title product (14.5 g, 74%) as a brown oil. LCMS (ESI): mass calcd. for C₁₄H₁₂BrNO 290.16 m/z, found 292.0 [M+H]⁺.

Step B. 5-Bromo-7-(3-bromophenyl)-6,7-dihydro-5H-cyclopenta[b]pyridin-7-ol. 1-Bromopyrrolidine-2,5-dione (8.37 g, 47.0 mmol) was added to a mixture of 7-(3-bromophenyl)-6,7-dihydro-5H-cyclopenta[b]pyridin-7-ol (13 g, 45 mmol), benzoic peroxyanhydride (2.17 g, 8.96 mmol) and CH₃CN (130 mL) at room temperature. The mixture was heated at 90° C. for 2 h. After this time, the mixture was cooled to room temperature and concentrated under vacuum to afford a residue, which was purified by

FCC (eluent: petroleum ether ethyl acetate=1:0 to 2:1) to give the title compound (7.7 g, 27%) as a brown oil. LCMS (ESI): mass calcd. For C₁₄H₁₁Br₂NO 369.05 m/z, found 369.9 [M+H]⁺.

Step C. 7-(3-Bromophenyl)-7-hydroxy-6,7-dihydro-5H-cyclopenta[b]pyridin-5-yl acetate. Sodium acetate (1.71 g, 20.9 mmol) was added to a mixture of 5-bromo-7-(3-bromophenyl)-6,7-dihydro-5H-cyclopenta[b]pyridin-7-ol (7.70 g, 20.8 mmol) in DMF (80 mL). The reaction mixture was then heated at 100° C. for 5 hours. After this time, the mixture was cooled to room temperature, quenched with water (200 mL), and extracted with ethyl acetate (150 mL×3). The combined organic extracts were washed with brine (300 mL), dried over Na₂SO₄, filtered, and concentrated to dryness under reduced pressure to give a residue, which was purified by FCC (eluent: petroleum ether:ethyl acetate=1:0 to 1:2) to afford the title product (7.9 g, 53%) as a yellow oil. LCMS (ESI): mass calcd. For C₁₆H₁₄Br₂NO₃ 347.01 m/z, found 348.0 [M+H]⁺.

Step D. 7-Hydroxy-7-(3-(4,4,5,5-tetramethyl-1,3,2-dioxaborolan-2-yl)phenyl)-6,7-dihydro-5H-cyclopenta[b]pyridin-5-yl acetate. A mixture of 7-(3-bromophenyl)-7-hydroxy-6,7-dihydro-5H-cyclopenta[b]pyridin-5-yl acetate (1.0 g, 2.9 mmol), 4,4,4′,4′,5,5,5′,5′-octamethyl-2,2′-bi(1,3,2-dioxaborolane) (1.46 g, 5.75 mmol), KOAc (846 mg, 8.62 mmol), and 1,4-dioxane (12 mL) was sparged with Ar for 5 min. 1,1-bis(di-tert-butylphosphino)ferrocene palladium dichloride (187 mg, 0.287 mmol) was added and the mixture was sparged with Ar for another 5 min, and then heated at 70° C. via microwave irradiation for 1 hour. After this time, the mixture was cooled to room temperature and concentrated under reduced pressure to give a residue, which was purified by FCC (eluent: petroleum ether. EtOAc=1:0 to 1:1) to afford the title compound (580 mg, 43%) as a yellow solid. LCMS (ESI): mass calcd. for C₂₂H₂₆BNO₅ 395.26 m/z, found 395.9 [M+H]⁺.

Step E. 7-Hydroxy-7-(3-(2-(1-(phenylsulfonyl)-1H-pyrrolo[2,3-b]pyridin-3-yl)thiazol-4-yl)phenyl)-6,7-dihydro-5H-cyclopenta[b]pyridin-5-yl acetate. To a microwave vial containing 7-hydroxy-7-(3-(4,4,5,5-tetramethyl-1,3,2-dioxaborolan-2-yl)phenyl)-6,7-dihydro-5H-cyclopenta[b]pyridin-5-yl acetate (530 mg, 1.34 mmol), 4-bromo-2-(1-(phenylsulfonyl)-1H-pyrrolo[2,3-b]pyridin-3-yl)thiazole (Intermediate 2564 mg, 1.34 mmol) and K₃PO₄ (854 mg, 4.02 mmol) was added 1,4-dioxane (12 mL). The mixture was sparged with Ar for 5 min. Then, 1,1′-bis(di-tert-butylphosphino)ferrocene palladium dichloride (87 mg, 0.133 mmol) was added to the mixture and the mixture was sparged with Ar for another 5 min and then heated at 100° C. via microwave irradiation for 1 h. After this time, the mixture was cooled to room temperature and filtered. The filtrate was diluted with water (30 mL) and extracted with EtOAc (50 mL×3). The combined organic extracts were dried over anhydrous Na₂SO₄, filtered, and concentrated to dryness under reduced pressure to afford the product (1.1 g, 50.5% purity, 68%) as a black solid. LCMS (ESI): mass calcd. for C₃₂H₂₄N₄O₅S₂ 608.11 m/z, found 609.1 [M+H]⁺.

### Intermediate 166. 7-(3-(2-(1H-Pyrrolo[2,3-b]pyridin-3-yl)thiazol-4-yl)phenyl)-6,7-dihydro-5H-cyclopenta[b]pyridine-5,7-diol

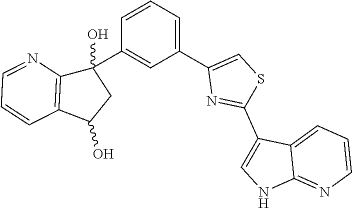 [SMILES: OC1CC(O)(c2cccc(-c3csc(-c4c[nH]c5ncccc45)n3)c2)c2ncccc21]

Sodium hydroxide (3.2 mL, 6.4 mmol, 2.0 M in water) was added to a solution of 7-hydroxy-7-(3-(2-(1-(phenylsulfonyl)-1H-pyrrolo[2,3-b]pyridin-3-yl)thiazol-4-yl)phenyl)-6,7 dihydro-5H-cyclopenta[b]pyridin-5-yl acetate (960 mg, 1.58 mmol) and 1,4-dioxane (15 mL). The mixture was heated at 60° C. for 2 h. After this time, the mixture was cooled to room temperature, and concentrated to dryness under reduced pressure to give the product, which was purified by preparative HPLC using a Phenomenex Gemini NX-C18, 75×30 mm×3 μm column (eluent: 17% to 47% (v/v) ACN and water (0.04% NH₃H₂O+10 mM NH₄HCO₃)) to afford a first eluting peak, Intermediate 166-A, which consisted of the two trans-alcohol diastereomers: trans-7-(3-(2-(1H-Pyrrolo[2,3-b]pyridin-3-yl)thiazol-4-yl)phenyl)-6,7-dihydro-5H-cyclopenta[b]pyridine-5,7-diol (33.8 mg) as a white solid. LCMS (ESI): mass calcd. for C₂₄H₁₈N₄O₂S 426.12 m/z, found 427.0 [M+H]⁺ and a second eluting peak (Intermediate 166-B) which consisted of the two cis-alcohol diastereomers: cis-7-(3-(2-(1H-Pyrrolo[2,3-b]pyridin-3-yl)thiazol-4-yl)phenyl)-6,7-dihydro-5H-cyclopenta[b]pyridine-5,7-diol as the second eluting product as a white solid (68 mg), LCMS (ESI): mass calcd. for C₂₄H₁₈N₄O₂S 426.12 m/z, found 427.0 [M+H]⁺.

### Intermediate 167. 5-Methyl-7-(3-(2-(1-(phenylsulfonyl)-1H-pyrrolo[2,3-b]pyridin-3-yl)thiazol-4-yl)phenyl)-7-((trimethylsilyl)oxy)-6,7-dihydro-5H-cyclopenta[b]pyridin-5-ol

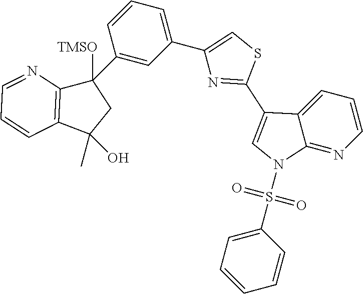

Step A. 7-(3-Bromophenyl)-6,7-dihydro-5H-cyclopenta[b]pyridine-5,7-diol. Sodium hydroxide (45.4 mL, 90.8 mmol, 2 M) was added to a mixture of 7-(3-bromophenyl)-7-hydroxy-6,7-dihydro-5H-cyclopenta[b]pyridin-5-yl acetate (Intermediate 165, Step C, 7.9 g, 22.7 mmol) in 1,4-dioxane (180 mL) and the mixture was stirred for 2 h at 60° C. After this time, the solvent was removed under reduced pressure to afford the product, which was purified by FCC (eluent: petroleum ether:ethyl acetate=1:0 to 0:1 to ethyl acetate:MeOH=9:1) to afford the title compound (3.5 g, 38%) as a brown solid. LCMS (ESI): mass calcd. For C₁₄H₁₂BrNO 306.16 m/z, found 308.0 [M+H]⁺.

Step B. 7-(3-Bromophenyl)-7-hydroxy-6,7-dihydro-5H-cyclopenta[b]pyridin-5-one. Manganese(IV) oxide (19.9 g, 229 mmol) was added to a solution of 7-(3-bromophenyl)-6,7-dihydro-5H-cyclopenta[b]pyridine-5,7-diol (3.5 g, 11 mmol) and CHCl₃ (100 mL). The resultant mixture was heated at 95° C. for 3 h. After this time, the mixture was filtered through a pad of diatomaceous earth and the pad was washed with ethyl acetate (100 mL). The solvent was removed under reduced pressure to afford product, which was purified by FCC (eluent: petroleum ether:ethyl acetate=1:0 to 1:1) to provide the title compound (1.4 g, 40%) as a yellow oil. LCMS (ESI): mass calcd. For C₁₄H₁₀BrNO₂ 302.98 m/z, found 304.0 [M+H]⁺.

Step C. 7-(3-Bromophenyl)-7-((trimethylsilyl)oxy)-6,7-dihydro-5H-cyclopenta[b]pyridin-5-one. Triethylamine (0.918 mL, 6.58 mmol) was added to a solution of 7-(3-bromophenyl)-7-hydroxy-6,7-dihydro-5H-cyclopenta[b]pyridin-5-one (200 mg, 0.658 mmol) and CH₂Cl₂ (3 mL). Chlorotrimethylsilane (0.835 mL, 6.58 mmol) was added to the mixture at 0° C. The reaction mixture was heated at 40° C. for 16 hours. After this time, methanol (3 mL) was added to the reaction mixture and the mixture stirred for 20 minutes. The mixture was concentrated to afford the product, which was purified by FCC (eluent: petroleum ether:ethyl acetate=1:0 to 1:2) to give the title compound (100 mg, 28%) as a yellow oil. LCMS (ESI): mass calcd. For C₁₇H₁₈BrNO₂Si 376.32 m/z, found 288.2 [M-88]⁺.

Step D. 7-(3-Bromophenyl)-5-methyl-7-((trimethylsilyl)oxy)-6,7-dihydro-5H-cyclopenta[b]pyridin-5-ol. Methylmagnesium bromide (2.39 mL, 3.0 M solution in 2-Me-THF, 7.18 mmol) was added dropwise to a 0° C. (water/ice) solution of 7-(3-bromophenyl)-7-((trimethylsilyl)oxy)-6,7-dihydro-5H-cyclopenta[b]pyridin-5-one (900 mg, 2.39 mmol) in anhydrous THE (10.0 mL). The resultant mixture was stirred at room temperature for 1 h. After this time, the mixture was quenched with water (30 mL) and extracted with ethyl acetate (30 mL×3). The combined organic extracts were washed with brine (60 mL), dried over Na₂SO₄, filtered, and concentrated to dryness under reduced pressure to afford the title compound (800 mg, 41.3%) as a yellow oil. LCMS (ESI): mass calcd. For C₁₈H₂₂BrNO₂Si 391.06 m/z, found 392.0 [M+H]⁺.

Step E. 5-Methyl-7-(3-(4,4,5,5-tetramethyl-1,3,2-dioxaborolan-2-yl)phenyl)-7-((trimethylsilyl)oxy)-6,7-dihydro-5H-cyclopenta[b]pyridin-5-ol. To a microwave vial containing 7-(3-bromophenyl)-5-methyl-7-((trimethylsilyl)oxy)-6,7-dihydro-5H-cyclopenta[b]pyridin-5-ol (750 mg, 1.91 mmol), (BPin)₂ (971 mg, 3.82 mmol) and potassium acetate (563 mg, 5.74 mmol) was added 1,4-dioxane (12 mL) under Ar. Then, [1,1′-bis(diphenylphosphino)ferrocene]dichloropalladium(II) (125 mg, 0.191 mmol) was added and the resultant mixture was sparged with Ar for 5 min and then heated at 70° C. via microwave irradiation for 1 hour. After this time, the mixture was cooled to room temperature, poured into water (30 mL), and extracted with ethyl acetate (30 mL×3). The combined organic extracts were washed with brine (60 mL), dried over Na₂SO₄, filtered, and concentrated to dryness under reduced pressure to give the product, which was purified by FCC (eluent: petroleum ether:ethyl acetate=1:0 to 1:1) to afford the title product (900 mg, 43%) as a brown oil. LCMS (ESI): 1.41 min, mass calcd. For C₂₄H₃₄BNO₄Si 439.23 m/z, found 440.1 [M+H]⁺.

Step F. 5-Methyl-7-(3-(2-(1-(phenylsulfonyl)-1H-pyrrolo[2,3-b]pyridin-3-yl)thiazol-4-yl)phenyl)-7-((trimethylsilyl)oxy)-6,7-dihydro-5H-cyclopenta[b]pyridin-5-ol. To a microwave vial containing 5-methyl-7-(3-(4,4,5,5-tetramethyl-1,3,2-dioxaborolan-2-yl)phenyl)-7-((trimethylsilyl)oxy)-6,7-dihydro-5H-cyclopenta[b]pyridin-5-ol (280 mg, 0.637 mmol) was added 4-bromo-2-(1-(phenylsulfonyl)-1H-pyrrolo[2,3-b]pyridin-3-yl)thiazole (Intermediate 2, 321 mg, 0.765 mmol), K₃PO₄ (406 mg, 1.91 mmol) and dioxane/water (4/1, 12 mL). The resultant mixture was sparged with Ar for 5 minutes and treated with [1,1′-bis(diphenylphosphino)ferrocene]dichloropalladium(II) (42 mg, 0.0064 mmol). The resultant mixture was sparged with Ar for another 5 min and the mixture was heated at 100° C. via microwave irradiation for 1 h. After this time, the mixture was cooled to room temperature, poured into water (30 mL), and extracted with ethyl acetate (30 mL×3). The combined organic extracts were washed with brine (60 mL), dried over Na₂SO₄, filtered, and concentrated to dryness under reduced pressure to afford the title product (600 mg) as black oil. LCMS (ESI): mass calcd. For C₃₄H₃₂N₄O₄S₂Si 652.16 m/z, found 653.2 [M+H]⁺.

### Intermediate 168. 7-(3-(2-(1H-pyrrolo[2,3-b]pyridin-3-yl)thiazol-4-yl)phenyl)-5-methyl-6,7-dihydro-5H-cyclopenta[b]pyridine-5,7-diol

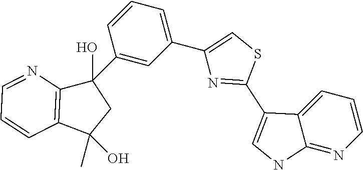 [SMILES: CC1(O)CC(O)(c2cccc(-c3csc(-c4c[nH]c5ncccc45)n3)c2)c2ncccc21]

Sodium hydroxide (2.95 mL, 5.90 mmol, 2 M) was added to a mixture of 5-methyl-7-(3-(2-(1-(phenylsulfonyl)-1H-pyrrolo[2,3-b]pyridin-3-yl)thiazol-4-yl)phenyl)-7-((trimethylsilyl)oxy)-6,7-dihydro-5H-cyclopenta[b]pyridin-5-ol (Intermediate 166, 550 mg, 0.842 mmol) in 1,4-dioxane (15 mL). The resultant mixture was stirred for 2 h at 60° C. After this time, the mixture was cooled to room temperature, and concentrated to dryness under reduced pressure to afford a residue, which was purified by FCC (eluent: petroleum ether:ethyl acetate=1:0 to 0:1 then CH₂Cl₂:MeOH=9:1) to afford the product as a black oil. The product was further purified by preparative HPLC using a Phenomenex Gemini NX-C18 75×30 mm×3 μm column (eluent: 26% to 56% (v/v) MeCN and water (0.05% NH₃H₂O+10 mM NH₄HCO₃)) to afford fractions. The first eliding diastereomer fraction provided the cis-alcohol diastereomers (cis)-(5R,7S)-7-(3-(2-(1H-pyrrolo[2,3-b]pyridin-3-yl)thiazol-4-yl)phenyl)-5-methyl-6,7-dihydro-5H-cyclopenta[b]pyridine-5,7-diol (35 mg, Yield: 8.9%) as a colorless solid and the second eliding diastereomer fraction provided the trans alcohol diastereomers (trans)-(5R,7R)-7-(3-(2-(1H-pyrrolo[2,3-b]pyridin-3-yl)thiazol-4-yl)phenyl)-5-methyl-6,7-dihydro-5H-cyclopenta[b]pyridine-5,7-diol (60 mg, Yield: 16%) as a colorless solid.

### Intermediate 169. (R)-3-(3-(2-(2,5-difluoro-1-tosyl-1H-pyrrolo[2,3-b]pyridin-3-yl)thiazol-4-yl)phenyl)-3-hydroxy-1-methylpyrrolidin-2-one

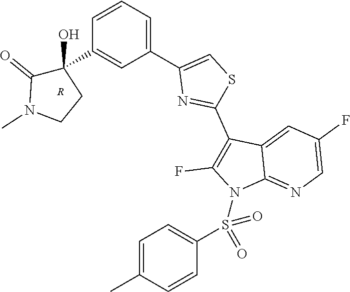

Step A. 3-Bromo-5 fluoro-1-tosyl-1H-pyrrolo[2,3-b]pyridine. Sodium hydride (483 mg, 60.0% purity, 12.1 mmol) was added to a 0° C. solution of 3-bromo-5-fluoro-1H-pyrrolo[2,3-b]pyridine (2.0 g, 9.3 mmol) and THF (15 mL). The reaction mixture was stirred at room temperature for 1 hour. Then, a solution of 4-methylbenzene-1-sulfonyl chloride (2.1 g, 11 mmol) in THF (5 mL) was added dropwise to the reaction at 0° C. The resultant mixture was stirred at 0° C. for 2 hours. After this time, the mixture was quenched with water (20 mL) and extracted with ethyl acetate (30 mL×2). The combined organic extracts were dried over anhydrous Na₂SO₄, filtered, and concentrated to dryness under reduced pressure to afford the product, which was purified by FCC (eluent: petroleum ether:ethyl acetate=1:0 to 1:1) to give the title compound (2.98 g, 85%) as a white solid. LCMS (ESI): mass calcd. for C₁₄H₁₀BrFN₂O₂S 367.96 m/z, found 368.9 [M+H]⁺.

Step B. 3-Bromo-2,5-difluoro-1-tosyl-1H-pyrrolo[2,3-b]pyridine. Lithium diisopropylamide (4.4 mL, 8.8 mmol, 2.0 M in THF) was added dropwise to a −72° C. (dry ice/ethanol) solution of 3-bromo-1-tosyl-1H-pyrrolo[2,3-b]pyridine (2.98 g, 8.07 mmol) in THF (35 mL) under N₂ atmosphere. The resultant mixture was stirred at −72° C. (dry ice/ethanol) for 1 hour and then treated with a solution of N-fluoro-N-(phenylsulfonyl)benzenesulfonamide (3.1 g, 9.7 mmol) in THF (15 mL) at −72° C. dropwise, and stirred for 16 hours with gradual warming to room temperature. After this time, the mixture was quenched with water (50 mL) and extracted with ethyl acetate (50 mL×3). The combined organic extracts were washed with brine (80 mL), dried over anhydrous Na₂SO₄, filtered, and concentrated under reduced pressure to give the product (4.0 g, 53% purity, 68%) as a brown solid. LCMS (ESI): mass calcd. for C₁₄H₉BrF₂N₂O₂S 385.95 m/z, found 386.9 [M+H]⁺.

Step C. (2,5-Difluoro-1-tosyl-1H-pyrrolo[2,3-b]pyridin-3-yl)boronic acid. n-Butyllithium (2.5 M solution in hexane, 4.1 mL, 10 mmol) was added dropwise to a −72° C. (dry ice/ethanol) solution of 3-bromo-2,5-difluoro-1-tosyl-1H-pyrrolo[2,3-b]pyridine (2.0 g, 5.2 mmol) and triisopropyl borate (2.4 mL, 10 mmol, 0.815 g/mL) in THF (20 mL). The resultant mixture was stirred at −72° C. (dry ice/ethanol) for 30 min. After this time, the mixture was quenched with water (10 mL), acidified with 1 M HCl to pH=6, and extracted with ethyl acetate (30 mL×2). The combined organic extracts were concentrated under reduced pressure to afford the product (2.4 g, 55% purity, 73%) as a brown oil, which was used for next step without further purification. LCMS (ESI): mass calcd. for C₁₄H₁₁BF₂N₂O₄S 352.05 m/z, found 352.8 [M+H]⁺.

Step D. (R)-3-(3-(2-(2,5-difluoro-1-tosyl-1H-pyrrolo[2,3-b]pyridin-3-yl)thiazol-4-yl)phenyl)-3-hydroxy-1-methylpyrrolidin-2-one. To a microwave vial containing (2,5-difluoro-1-tosyl-1H-pyrrolo[2,3-b]pyridin-3-yl)boronic acid (449 mg, 0.708 mmol, 55.5% purity), (R)-3-(3-(2-bromothiazol-4-yl)phenyl)-3-hydroxy-1-methylpyrrolidin-2-one (Intermediate 187, 200 mg, 0.566 mmol), K₃PO₄ (451 mg, 2.12 mmol) and 1,1-bis(di-tert-butylphosphino)ferrocene palladium dichloride (46 mg, 0.071 mmol) was added 1,4-dioxane/H₂O (4/1, 6 mL,) under N₂ atmosphere. The resultant mixture was heated at 80° C. via microwave irradiation for 1 hour. After this time, the mixture was cooled to room temperature and concentrated under reduced pressure to afford the product, which was purified by FCC (eluent: petroleum ether:ethyl acetate=1:0 to 0:1) to afford the title compound (320 mg, 25% purity, 20%) as a brown oil. LCMS (ESI): mass calcd. for C₂₈H₂₂F₂N₄O₄S₂ 580.11 m/z, found 581.1 [M+H]⁺.

### Intermediate 170. 4-Bromo-2-(1H-pyrrolo[3,2-b]pyridin-1-yl)thiazole

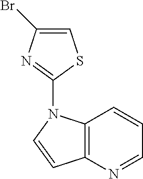 [SMILES: Brc1csc(-n2ccc3ncccc32)n1]

1H-Pyrrolo[3,2-b]pyridine (1.0 g, 8.5 mmol) was added to a mixture of 2,4-dibromothiazole (2.5 g, 10 mmol), Cs₂CO₃ (6.9 g, 21 mmol), and DMF (25 mL). The resultant mixture was sparged with N₂ for 5 minutes and then treated with CuI (1.6 g, 8.4 mmol). The resultant mixture was sparged with N₂ for another 5 minutes and then heated at 120° C. for 16 hours. After this time, the mixture was cooled to room temperature and filtered. The filtrate was diluted with saturated NH₄Cl (10 mL) and extracted with ethyl acetate (30 mL×3). The combined organic extracts were washed with brine (10 mL), dried over anhydrous Na₂SO₄, filtered, and concentrated under reduced pressure to afford the product, which was purified by FCC (eluent: petroleum ether:ethyl acetate=1:0 to 1:1) to afford the title compound (900 mg, 30%) as a white solid. LCMS (ESI): mass calcd. for C₁₀H₆BrN₃S 278.95 m/z, found 281.8 [M+1]⁺.

### Intermediate 171. (R)-3-Hydroxy-1-methyl-3-(3-(5-(1-(phenylsulfonyl)-1H-pyrrolo[2,3-b]pyridin-3-yl)furan-2-yl)phenyl)pyrrolidin-2-one

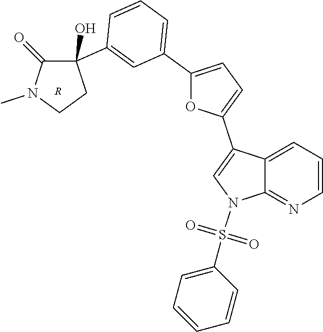

Step A. (R)-3-(3-(5-Bromofuran-2-yl)phenyl)-3-hydroxy-1-methylpyrrolidin-2-one. To a microwave vial containing (R)-3-Hydroxy-1-methyl-3-(3-(4,4,5,5-tetramethyl-1,3,2-dioxaborolan-2-yl)phenyl)pyrrolidin-2-one (Intermediate 16, 300 mg, 0.946 mmol), 2,5-dibromofuran (0.1 mL, 0.93 mmol) and K₂CO₃ (392 mg, 2.84 mmol) was added 1,4-dioxane (6 mL), and H₂O (1.5 mL). The mixture was sparged with Ar for 5 minutes and then treated with Pd(dppf)Cl₂ (69 mg, 0.094 mmol). The mixture was sparged with Ar for another 5 minutes and then heated at 80° C. via microwave irradiation for 1 hour. After this time, the mixture was cooled to room temperature and concentrated under reduced pressure to give the product, which was purified by FCC (eluent: petroleum ether:ethyl acetate=1:0 to 0:1) to afford the title compound (100 mg, 19%) as a yellow oil. LC-MS (ESI): mass calcd. For C₁₅H₁₄BrNO₃ 335.02 m/z found 336.0 [M+H]⁺.

Step B. (R)-3-Hydroxy-1-methyl-3-(3-(5-(1-(phenylsutfonyl)-1H-pyrrolo[2,3-b]pyridin-3-yl)furan-2-yl)phenyl)pyrrolidin-2-one. To a microwave vial containing (R)-3-(3-(5-bromofuran-2-yl)phenyl)-3-hydroxy-1-methylpyrrolidin-2-one (240 mg, 0.714 mmol), 1-(phenylsulfonyl)-3-(4,4,5,5-tetramethyl-1,3,2-dioxaborolan-2-yl)-1H-pyrrolo[2,3-b]pyridine (274 mg, 0.713 mmol) and K₃PO₄ (454 mg, 2.14 mmol) was added 1,4-dioxane (6 mL), and H₂O (1.5 mL). The mixture was sparged with Ar for 5 minutes and then treated with Pd(dtbpf)Cl₂ (46 mg, 0.071 mmol). The mixture was sparged with Ar for another 5 minutes and then heated at 80° C. via microwave irradiation for 1 hour. After this time, the mixture was cooled to room temperature and concentrated under reduced pressure to give the product, which was purified by FCC (eluent: petroleum ether:ethyl acetate=1:0 to 0:1) to afford the title compound (280 mg, 52%) as a brown oil. LC-MS (ESI): mass calcd. For C₂₈H₂₃N₃O₅S 513.14 m/z found 514.1 [M+H]⁺.

### Intermediate 172. (R)-3-Hydroxy-1-methyl-3-(3-(4-(1-(phenylsulfonyl)-1H-pyrrolo[2,3-b]pyridin-3-yl)furan-2-yl)phenyl)pyrrolidin-2-one

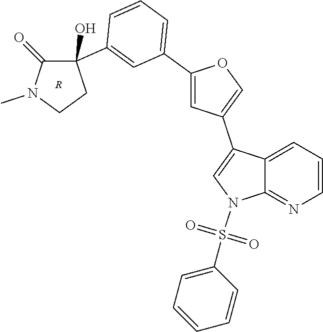

Step A. (R)-3-(3-(4-Bromofuran-2-yl)phenyl)-3-hydroxy-1-methylpyrrolidin-2-one. To a microwave vial containing (R)-3-(3-Bromophenyl)-3-hydroxy-1-methylpyrrolidin-2-one (250 mg, 0.926 mmol), 2-(4-bromofuran-2-yl)-4,4,5,5-tetramethyl-1,3,2-dioxaborolane (303 mg, 1.11 mmol) and K₂CO₃ (384 mg, 2.78 mmol) was added 1,4-dioxane (10 mL), and H₂O (2 mL). The mixture was sparged with Ar for 5 minutes and then treated with Pd(dppf)Cl₂ (68 mg, 0.09 mmol). The mixture was sparged with Ar for another 5 minutes and then heated at 90° C. via microwave irradiation for 1 hour. After this time, the mixture was cooled to room temperature and filtered. The filtrate was concentrated to dryness under reduced pressure to give the product, which was purified by FCC (eluent: petroleum ether:ethyl acetate=10:1 to 1:1) to afford the title compound (220 mg, 31%) as a clear oil. LCMS (ESI): mass calcd. for C₁₅H₁₄BrNO₃ 335.02 m/z, found 336.0 [M+H]⁺.

Step B. (R)-3-hydroxy-1-methyl-3-(3-(4-(1-(phenylsulfonyl)-1H-pyrrolo[2,3-b]pyridin-3-yl)furan-2-yl)phenyl)pyrrolidin-2-one. To a microwave vial containing (R)-3-(3-(4-bromofuran-2-yl)phenyl)-3-hydroxy-1-methylpyrrolidin-2-one (200 mg, 0.595 mmol), 1-(phenylsulfonyl)-3-(4,4,5,5-tetramethyl-1,3,2-dioxaborolan-2-yl)-1H-pyrrolo[2,3-b]pyridine (274 mg, 0.713 mmol) and K₃PO₄ (379 mg, 1.79 mmol) was added 1,4-dioxane (10 mL), and H₂O (2 mL). The mixture was sparged with Ar for 5 minutes and then treated with Pd(dtbpf)Cl₂ (39 mg, 0.06 mmol). The mixture was sparged with Ar for another 5 minutes and then heated at 80° C. via microwave irradiation for 1 hour. After this time, the mixture was cooled to room temperature and concentrated to dryness under reduced pressure to afford the product, which was purified by FCC (eluent: petroleum ether:ethyl acetate=10:1 to 0:1) to afford the title compound (220 mg, 32%) as a brown solid. LCMS (ESI): mass calcd. for C₂₈H₂₃N₃O₅S 513.14 m/z, found 514.1 [M+H]⁺.

### Intermediate 173. (R)-3-Hydroxy-1-methyl-3-(3-(2-(1-tosyl-1H-pyrrolo[2,3-b]pyridin-3-yl)oxazol-4-yl)phenyl)pyrrolidin-2-one

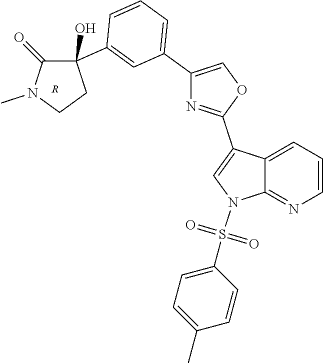

Step A. 4-Bromo-2-(1-tosyl-1H-pyrrolo[2,3-b]pyridin-3-yl)oxazole. 3-(4,4,5,5-Tetramethyl-1,3,2-dioxaborolan-2-yl)-1-tosyl-1H-pyrrolo[2,3-b]pyridine (1.0 g, 2.5 mmol), 2,4-dibromooxazole (0.38 mL, 3.8 mmol), K₃PO₄ (1.6 g, 7.5 mmol), 1,4-dioxane (8 mL), and H₂O (2 mL) were added to a 50 mL flask. The resultant mixture was sparged with Ar for 5 minutes and then treated with Pd(dppf)Cl₂ (184 mg, 0.251 mmol). The mixture was sparged with Ar for another 5 minutes and then heated at 80° C. for 3 hours. After this time, the mixture was cooled to room temperature and concentrated to dryness under reduced pressure to give the product, which was purified by FCC (eluent: petroleum ether. ethyl acetate=1:0 to 5:1) to give the title compound (551 mg, 50%) as a white solid. LCMS (ESI): mass calcd. for C₁₇H₁₂BrN₃O₃S 416.98 m/z, found 417.9 [M+H]⁺. ¹H NMR (400 MHz, DMSO-d₆) δ 8.55-8.43 (m, 4H), 8.10 (d, J=8.4 Hz, 2H), 7.49-7.42 (m, 3H), 2.35 (s, 3H).

Step B. (R)-3-Hydroxy-1-methyl-3-(3-(2-(1-tosyl-1H-pyrrolo[2,3-b]pyridin-3-yl)oxazol-4-yl)phenyl)pyrrolidin-2-one. To a microwave vial containing 4-bromo-2-(1-tosyl-1H-pyrrolo[2,3-b]pyridin-3-yl)oxazole (475 mg, 1.14 mmol), (R)-3-hydroxy-1-methyl-3-(3-(4,4,5,5-tetramethyl-1,3,2-dioxaborolan-2-yl)phenyl)pyrrolidin-2-one (Intermediate 16, 300 mg, 0.946 mmol) and K₃PO₄ (602 mg, 2.84 mmol) was added 1,4-dioxane (2.4 mL), and H₂O (0.6 mL). The resultant mixture was sparged with Ar for 5 minutes and then treated with Pd(dppf)Cl₂ (69 mg, 0.095 mmol). The mixture was sparged with Ar for another 5 minutes and then heated at 80° C. via microwave for 1 hour. After this time, the mixture was cooled to room temperature and concentrated to dryness under reduced pressure to give the product, which was purified by FCC (eluent: petroleum ether:ethyl acetate=1:0 to 0:1) to give the title compound (432 mg, 82%) as a yellow solid. LCMS (ESI): mass calcd. for C₂₈H₂₄N₄O₅S 528.15 m/z, found 529.1 [M+1]⁺.

### Intermediate 174. (R)-3-(3-(5-(1H-Pyrrolo[2,3-b]pyridin-3-yl)-1-((2-(trimethylsilyl)ethoxy)methyl)-1H-pyrazol-3-yl)phenyl)-3-hydroxy-1-methylpyrrolidin-2-one

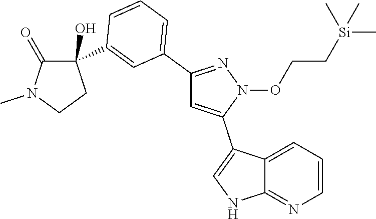

Step A. 3,5-Dibromo-1-((2-(trimethylsilyl)ethoxy)methyl)-1H-pyrazole. 2-(Trimethylsilyl)ethoxymethyl chloride (6.46 g, 38.7 mmol) was added to a solution of 3,5-dibromo-1H-pyrazole (2.5 g, 11 mmol), Cs₂CO₃ (36.1 g, 66.4 mmol), and acetone (70 mL). The resultant mixture was stirred at room temperature for 16 hours. After this time, the mixture was poured into water (100 mL) and extracted with ethyl acetate (100 mL×3). The combined organic extracts were washed with brine (200 mL), dried over anhydrous Na₂SO₄, filtered, and concentrated under reduced pressure to afford the product, which was purified by FCC (eluent: petroleum ether:ethyl acetate=1:0 to 1:1) to give the title compound (3.8 g, 89%) as a yellow oil. ¹H NMR (400 MHz, CDCl₃) δ 6.42-6.36 (m, 1H), 5.45 (s, 2H), 3.66-3.62 (m, 2H), 1.03-0.95 (m, 2H), 0.02 (s, 9H).

Step B. 3-(3-Bromo-1-((2-(trimethylsilyl)ethoxy)methyl)-1H-pyrazol-5-yl)-1-tosyl-1H-pyrrolo[2,3-b]pyridine. To a microwave vial containing 3-(4,4,5,5-tetramethyl-1,3,2-dioxaborolan-2-yl)-1-tosyl-1H-pyrrolo[2,3-b]pyridine (1.5 g, 3.8 mmol), 3,5-dibromo-1-((2-(trimethylsilyl)ethoxy)methyl)-1H-pyrazole (1.34 g, 3.77 mmol) and potassium phosphate (2.40 g, 11.3 mmol) was added 1,4-dioxane/H₂O (15 mL, 4/1). The resultant mixture was sparged with N₂ for 5 minutes and then treated with Xphos-Pd-G₂ (296 mg, 0.377 mmol). The mixture was sparged with N₂ for another 5 minutes and then heated at 100° C. via microwave irradiation for 1 hour. After this time, the mixture was cooled to room temperature and combined with another identical reaction mixture. The combined mixtures were diluted with water (50 mL) and extracted with ethyl acetate (50 mL×3). The organic extracts were washed with brine (100 mL), dried over anhydrous Na₂SO₄, filtered, and concentrated under reduced pressure to give the product, which was purified by FCC (eluent: petroleum ether ethyl acetate=1:0 to 1:1) to afford the title compound (2.2 g, 57% purity, 30%) as a brown oil. LCMS (ESI): mass calcd. for C₂₃H₂₇BrN₄O₃SSi 548.07 m/z, found 549.0 [M+H]⁺.

Step C. (R)-3-Hydroxy-1-methyl-3-(3-(5-(1-tosyl-1H-pyrrolo[2,3-b]pyridin-3-yl)-1-((2-(trimethylsilyl)ethoxy)methyl)-1H-pyrazol-3-yl)phenyl)pyrrolidin-2-one. To a microwave vial containing 3-(3-bromo-1-((2-(trimethylsilyl)ethoxy)methyl)-1H-pyrazol-5-yl)-1-tosyl-1H-pyrrolo[2,3-b]pyridine (1 g, 1 mmol), (R)-3-hydroxy-1-methyl-3-(3-(4,4,5,5-tetramethyl-1,3,2-dioxaborolan-2-yl)phenyl)pyrrolidin-2-one (Intermediate 16, 396 mg, 1.25 mmol) and potassium phosphate (663 mg, 3.12 mmol) was added dioxane/H₂O (4/1, 15 mL,). The resultant mixture was sparged with N₂ for 5 minutes and then treated with Xphos-Pd-G₂ (81.9 mg, 0.104 mmol). The mixture was sparged with N₂ for another 5 minutes and then heated at 100° C. via microwave irradiation for 1 hour. After this time, the mixture was diluted water (50 mL) and extracted with ethyl acetate (50 mL×3). The combined organic extracts were washed with brine (100 mL), dried over anhydrous Na₂SO₄, filtered, and concentrated under reduced pressure to give the product, which was purified by FCC (eluent: petroleum ether:ethyl acetate=1:0 to 0:1) to afford the title compound (400 mg, 93% purity, 54%) as a brown oil. LCMS (ESI): mass calcd. for C₃₄H₃₉N₅O₅SSi 657.24 m/z, found 658.1 [M+H]⁺.

Step D. (R)-3-(3-(5-(1H-Pyrrolo[2,3-b]pyridin-3-yl)-1-((2-(trimethylsilyl)ethoxy)methyl)-1H-pyrazol-3-yl)phenyl)-3-hydroxy-1-methylpyrrolidin-2-one. Tetrabutylammonium fluoride (5.32 mL, 1.0 M in THF, 5.32 mmol) was added to of 3-hydroxy-1-methyl-3-(3-(5-(1-tosyl-1H-pyrrolo[2,3-b]pyridin-3-yl)-1-((2-(trimethylsilyl)ethoxy)methyl)-1H-pyrazol-3-yl)phenyl)pyrrolidin-2-one (350 mg, 0.532 mmol) in THF (10 mL). The resultant mixture was heated at 90° C. for 16 hours. After this time, the mixture was cooled to room temperature and concentrated under reduced pressure to give the product, which was purified by FCC (eluent: methylene chloride:methanol=1:0 to 9:1) to afford the title compound (260 mg, 64%) as a brown oil. ¹H NMR (400 MHz, CDCl₃) δ 10.89 (br s, 1H), 8.50 (d, J=4.0 Hz, 1H), 7.96 (s, 1H), 7.90 (d, J=7.7 Hz, 1H), 7.84 (d, J=8.2 Hz, 1H), 7.78-7.69 (m, 2H), 7.48-7.39 (m, 1H), 7.28-7.27 (m, 1H), 7.19-7.12 (m, 1H), 5.68 (s, 1H), 5.48-5.35 (m, 2H), 3.84-3.72 (m, 2H), 3.71-3.66 (m, 1H), 3.64-3.55 (m, 1H), 3.07 (s, 3H), 2.80-2.62 (m, 2H), 1.00-0.89 (m, 2H), 0.03-−0.05 (m, 9H).

### Intermediate 175. (R)-3-Hydroxy-1-methyl-3-(3-(1-methyl-3-(1-(phenylsulfonyl)-1H-pyrrolo[2,3-b]pyridin-3-yl)-1H-pyrazol-5-yl)phenyl)pyrrolidin-2-one

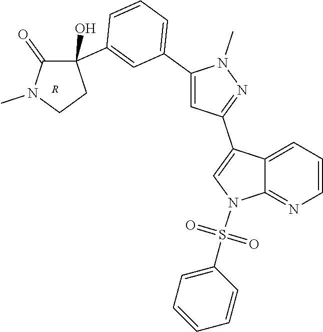

Step A. (R)-3-(3-(3-Bromo-1-methyl-1H-pyrazol-5-yl)phenyl)-3-hydroxy-1-methylpyrrolidin-2-one. To a microwave vial containing (R)-3-Hydroxy-1-methyl-3-(3-(4,4,5,5-tetramethyl-1,3,2-dioxaborolan-2-yl)phenyl)pyrrolidin-2-one (Intermediate 16, 180 mg, 0.567 mmol), 3,5-dibromo-1-methyl-1H-pyrazole (100 μL, 0.834 mmol) and K₃PO₄ (360 mg, 1.70 mmol) was added 1,4-dioxane (2.8 mL), and H₂O (0.7 mL). The resultant mixture was sparged with N₂ for 5 minutes and then treated with Pd(dppf)Cl₂ (45 mg, 0.062 mmol). The mixture was sparged with N₂ for another 5 minutes and then heated at 80° C. via microwave irradiation for 1 hour. After this time, the mixture was cooled to room temperature and concentrated to dryness under reduced pressure to afford the product which was purified by FCC (eluent: petroleum ether:ethyl acetate=1:0 to 0:1) to afford the title compound (129 mg, 61%) as a brown solid. LCMS (ESI): mass calcd. for C₁₅H₁₆BrN₃O₂ 349.04 m/z, found 350.0 [M+H]⁺. ¹H NMR (400 MHz, DMSO-d₆) δ 8.62-8.59 (m, 1H), 8.42-8.38 (m, 1H), 8.35 (s, 1H), 8.13-8.08 (m, 2H), 7.71-7.66 (m, 1H), 7.63-7.57 (m, 2H), 7.55-7.52 (m, 1H), 7.50-7.46 (m, 2H), 7.44-7.40 (m, 1H), 7.40-7.36 (m, 1H), 7.02 (s, 1H), 6.12 (s, 1H), 3.89 (s, 3H), 3.50-3.39 (m, 1H), 3.39-3.33 (m, 1H), 2.82 (s, 3H), 2.40-2.31 (m, 1H), 2.31-2.19 (m, 1H).

Step B. (R)-3-Hydroxy-1-methyl-3-(3-(1-methyl-3-(1-(phenylsulfonyl)-1H-pyrrolo[2,3-b]pyridin-3-yl)-1H-pyrazol-5-yl)phenyl)pyrrolidin-2-one. To a microwave vial containing (R)-3-(3-(3-Bromo-1-methyl-1H-pyrazol-5-yl)phenyl)-3-hydroxy-1-methylpyrrolidin-2-one (180 mg, 0.514 mmol), 1-(phenylsulfonyl)-3-(4,4,5,5-tetramethyl-1,3,2-dioxaborolan-2-yl)-1H-pyrrolo[2,3-b]pyridine (382 mg, 0.616 mmol) and K₃PO₄ (333 mg, 1.57 mmol) were added 1,4-dioxane (4 mL) and H₂O (1 mL). The resultant mixture was sparged with N₂ for 5 minutes and then treated with Pd(dtbpf)Cl₂ (36 mg, 0.055 mmol). The mixture was sparged with N₂ for another 5 minutes and then heated at 80° C. via microwave irritation for 1 hour. After this time, the mixture was cooled to room temperature and was concentrated to dryness under reduced pressure to afford the product which was purified by FCC (methylene chloride:methanol=1:0 to 9:1) to afford the title compound (316 mg) as a brown solid. ¹H NMR (400 MHz, DMSO-d₆) δ 8.62-8.59 (m, 1H), 8.42-8.38 (m, 1H), 8.35 (s, 1H), 8.13-8.08 (m, 2H), 7.71-7.66 (m, 1H), 7.63-7.57 (m, 2H), 7.55-7.52 (m, 1H), 7.50-7.46 (m, 2H), 7.44-7.40 (m, 1H), 7.40-7.36 (m, 1H), 7.02 (s, 1H), 6.12 (s, 1H), 3.89 (s, 3H), 3.50-3.39 (m, 1H), 3.39-3.33 (m, 1H), 2.82 (s, 3H), 2.40-2.31 (m, 1H), 2.31-2.19 (m, 1H).

### Intermediate 176. (R)-3-Hydroxy-1-methyl-3-(3-(4-methyl-5-(1H-pyrrolo[2,3-b]pyridin-3-yl)-1-((2-(trimethylsilyl)ethoxy)methyl)-1H-pyrazol-3-yl)phenyl)pyrrolidin-2-one

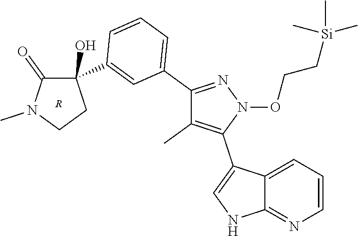

Step A. 3,5-Dibromo-4-methyl-1H-pyrazole. n-Butyllithium (2.5 M solution in hexane, 17.3 mL, 43.3 mmol) was added dropwise to a −72° C. (dry ice/ethanol) solution of 3,4,5-tribromo-1H-pyrazole (6.00 g, 19.7 mmol) and THE (100 mL). The resultant mixture was stirred at −72° C. for 30 minutes, and then treated with iodomethane (2.79 g, 19.7 mmol) dropwise. The mixture was stirred for 1 hour with gradual warming to room temperature. After this time, The mixture was quenched with saturated NH₄Cl (20 mL), diluted with water (80 mL), and extracted with ethyl acetate (100 mL×3). The combined organic extracts were washed with brine (200 mL), dried over anhydrous Na₂SO₄, filtered, and concentrated to dryness under reduced pressure to afford the title compound (4.2 g, 42%) as a white solid. LCMS (ESI): mass calcd. for C₄H₄Br₂N₂ 239.90 m/z, found 240.9 [M+H]⁺.

Step B. 3,5-Dibromo-4-methyl-1-((2-(trimethylsilyl)ethoxy)methyl)-1H-pyrazole. 2-(Trimethylsilyl)ethoxymethyl chloride (10.2 g, 61.3 mmol) was added to a mixture of 3,5-dibromo-4-methyl-1H-pyrazole (4.2 g, 18 mmol), Cs₂CO₃ (57.0 g, 105 mmol), and acetone (120 mL). The resultant mixture was stirred at room temperature for 2 hours. After this time, the mixture was poured into water (100 mL) and extracted with ethyl acetate (100 mL×3). The combined organic extracts were washed with brine (200 mL), dried over anhydrous Na₂SO₄, filtered, and concentrated under reduced pressure to afford the product, which was purified by FCC (eluent: petroleum ether:ethyl acetate=1:0 to 9:1) to afford the title compound (5.8 g, 60% purity, 52%) as a colorless oil. LCMS (ESI): mass calcd. for C₁₀H₁₈Br₂N₂OSi 370.16 m/z, found 370.9 [M+H]⁺.

Step C. 3-(3-Bromo-4-methyl-1-((2-(trimethylsilyl)ethoxy)methyl)-1H-pyrazol-5-yl)-1-tosyl-1H-pyrrolo[2,3-b]pyridine. To a microwave vial containing 3,5-dibromo-4-methyl-1-((2-(trimethylsilyl)ethoxy)methyl)-1H-pyrazole (500 mg, 0.810 mmol), 3-(4,4,5,5-tetramethyl-1,3,2-dioxaborolan-2-yl)-1-tosyl-1H-pyrrolo[2,3-b]pyridine (387 mg, 0.973 mmol), K₃PO₄ (516 mg, 2.43 mmol), and Xphos-Pd-G₂ (64 mg, 0.081 mmol) was added 1,4-dioxane/H₂O (4/1, 5 mL,). The resultant mixture was sparged with N₂ for 5 minutes and then heated to 100° C. via microwave irradiation for 1 hour. After this time, the mixture was cooled to room temperature, poured into water (10 mL), and extracted with ethyl acetate (10 mL×3). The combined organic extracts were washed with brine (10 mL), dried over anhydrous Na₂SO₄, filtered, and concentrated under reduced pressure to give the product, which was purified by FCC (eluent: petroleum ether:ethyl acetate=1:0 to 9:1) to afford the title compound (250 mg, 38%) as a yellow oil. LCMS (ESI): mass calcd. for C₂₄H₂₉BrN₄O₃SSi 560.09 m/z, found 561.1 [M+1]⁺. ¹H NMR (400 MHz, CDCl₃) δ 8.57-8.46 (m, 1H), 8.19-8.15 (m, 1H), 8.13-8.06 (m, 1H), 8.05-7.96 (m, 1H), 7.78-7.70 (m, 1H), 7.37-7.30 (m, 2H), 7.28-7.24 (m, 1H), 5.63-5.14 (m, 2H), 3.84-3.62 (m, 2H), 2.43-2.38 (m, 3H), 1.29 (t, J=7.2 Hz, 3H), 1.05-0.93 (m, 2H), 0.10-−0.02 (m, 9H).

Step D. (3R)-3-Hydroxy-1-methyl-3-(3-(4-methyl-5-(1-tosyl-1H-pyrrolo[2,3-b]pyridin-3-yl)-1-((2-(trimethylsilyl)ethoxy)methyl)-1H-pyrazol-3-yl)phenyl)pyrrolidin-2-one. To a microwave vial containing 3-(3-bromo-4-methyl-1-((2-(trimethylsilyl)ethoxy)methyl)-1H-pyrazol-5-yl)-1-tosyl-1H-pyrrolo[2,3-b]pyridine (1.0 g, 1.4 mmol), (R)-3-hydroxy-1-methyl-3-(3-(4,4,5,5-tetramethyl-1,3,2-dioxaborolan-2-yl)phenyl)pyrrolidin-2-one (Intermediate 16, 745 mg, 1.67 mmol), K₃PO₄ (884 mg, 4.17 mmol) and Xphos-Pd-G₂ (109 mg, 0.139 mmol) was added 1,4-dioxane/H₂O (4:1, 10 mL,). The resultant mixture was sparged with Ar for 5 minutes, and then heated at 100° C. via microwave irradiation for 1 hour. After this time, the mixture was cooled to room temperature, poured into water (15 mL), and extracted with ethyl acetate (20 mL×3). The combined organic extracts were washed with brine (20 mL), dried over anhydrous Na₂SO₄, filtered, and concentrated under reduced pressure to give the product, which was purified by FCC (eluent: petroleum ether:ethyl acetate=1:0 to 1:1) to afford the title compound (720 mg, 49%) as a yellow oil. LCMS (ESI): min, mass calcd. for C₃₅H₄₁N₅O₅SSi 671.26 m/z, found 672.3 [M+1]⁺.

Step E. (3R)-3-Hydroxy-1-methyl-3-(3-(4-methyl-5-(1H-pyrrolo[2,3-b]pyridin-3-yl)-1-((2-(trimethylsilyl)ethoxy)methyl)-1H-pyrazol-3-yl)phenyl)pyrrolidin-2-one. Sodium hydroxide (3.1 mL, 2.0 M in H₂O, 6.3 mmol) was added to a solution of (3R)-3-hydroxy-1-methyl-3-(3-(4-methyl-5-(1-tosyl-1H-pyrrolo[2,3-b]pyridin-3-yl)-1-((2-(trimethylsilyl)ethoxy)methyl)-1H-pyrazol-3-yl)phenyl)pyrrolidin-2-one (700 mg, 1.04 mmol) and 1,4-dioxane (10 mL). The resultant mixture was heated at 60° C. for 3 hours. After this time, the mixture was cooled to room temperature, poured into water (10 mL), and extracted with ethyl acetate (15 mL×3). The combined organic extracts was dried over anhydrous Na₂SO₄, filtered, and concentrated to dryness under reduced pressure to give the title compound (550 mg, 88%) as a yellow oil. LCMS (ESI): mass calcd. for C₂₈H₃₅N₅O₃Si 517.25 m/z, found 518.1 [M+1]⁺.

### Intermediate 177. (R)-3-Hydroxy-1-methyl-3-(3-(1-methyl-2-(1-tosyl-1H-pyrrolo[2,3-b]pyridin-3-yl)-1H-imidazol-5-yl)phenyl)pyrrolidin-2-one

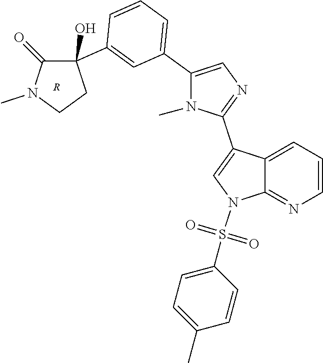

Step A. 3-(5-Bromo-1-methyl-1H-imidazol-2-yl)-1-tosyl-1H-pyrrolo[2,3-b]pyridine. To a microwave vial containing 3-(4,4,5,5-Tetramethyl-1,3,2-dioxaborolan-2-yl)-1-tosyl-1H-pyrrolo[2,3-b]pyridine (500 mg, 1.26 mmol), 2,5-dibromo-1-methyl-1H-imidazole (360 mg, 1.50 mmol) and K₃PO₄ (800 mg, 3.77 mmol) were added 1,4-dioxane (3.2 mL) and H₂O (0.8 mL). The resultant mixture was sparged with N₂ for 5 minutes and then treated with Pd(dppf)Cl₂ (95 mg, 0.13 mmol). The mixture was sparged with N₂ for another 5 minutes and then heated at 80° C. via microwave irradiation for 1 hour. After this time, the mixture was cooled to room temperature and concentrated to dryness under reduced pressure to afford the product, which was purified by FCC (eluent: petroleum ether:ethyl acetate=1:0 to 2:1) to afford the title compound (200 mg, 34%) as a white solid. LCMS (ESI): mass calcd. for C₁₈H₁₅BrN₄O₂S 430.01 m/z, found 431.0 [M+H]⁺. ¹H NMR (400 MHz, DMSO-d₆) δ 8.49-8.45 (m, 1H), 8.42-8.39 (m, 1H), 8.26 (s, 1H), 8.03 (d, J=8.6 Hz, 2H), 7.39 (d, J=7.9 Hz, 2H), 7.35 (d, J=7.9 Hz, 1H), 7.20 (s, 1H), 3.80 (s, 3H), 2.30 (s, 3H).

Step B. (R)-3-Hydroxy-1-methyl-3-(3-(1-methyl-2-(1-tosyl-1H-pyrrolo[2,3-b]pyridin-3-yl)-1H-imidazol-5-yl)phenyl)pyrrolidin-2-one. To a microwave vial containing (R)-3-Hydroxy-1-methyl-3-(3-(4,4,5,5-tetramethyl-1,3,2-dioxaborolan-2-yl)phenyl)pyrrolidin-2-one (Intermediate 16, 120 mg, 0.378 mmol), 3-(5-bromo-1-methyl-1H-imidazol-2-yl)-1-tosyl-1H-pyrrolo[2,3-b]pyridine (168 mg, 0.390 mmol), and K₃PO₄ (240 mg, 1.13 mmol) were added 1,4-dioxane (2.4 mL) and H₂O (0.6 mL). The resultant mixture was sparged with N₂ for 5 minutes and then treated with Pd(dppf)Cl₂ (30 mg, 0.041 mmol). The mixture was sparged with N₂ for another 5 minutes and then heated at 80° C. via microwave irritation for 1 hour. After this time, the mixture was cooled to room temperature and concentrated to dryness under reduced pressure to afford the product which was purified by FCC (eluent: petroleum ether:ethyl acetate=1:0 to 0:1, and then ethyl acetate:methanol=1:0 to 10:1) to afford the title compound (166 mg, 60%) as a brown solid. LCMS (ESI): mass calcd. for C₂₉H₂₇N₅O₄S 541.18 m/z, found 542.2 [M+H]⁺.

### Intermediate 178. 3-Hydroxy-1-methyl-3-(3-(3-(1-tosyl-1H-pyrrolo[2,3-b]pyridin-3-yl)-1H-1,2,4-triazol-1-yl)phenyl)pyrrolidin-2-one

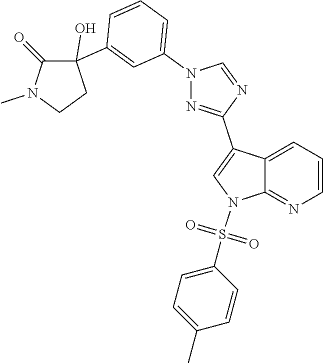 [SMILES: Cc1ccc(S(=O)(=O)n2cc(-c3ncn(-c4cccc(C5(O)CCN(C)C5=O)c4)n3)c3cccnc32)cc1]

Step A. 3-Hydroxy-3-(3-iodophenyl)-1-methylpyrrolidin-2-one. n-Butyllithium (27.6 mL, 2.5 M in hexane, 69.0 mmol) was added to a −72° C. solution of 1,3-diiodobenzene (22.7 g, 69.0 mmol) and THF (60 mL). The resultant mixture was stirred at −72° C. for 20 minutes and then treated with 1-methylpyrrolidine-2,3-dione (6.0 g, 53 mmol, in 120 mL THF), and stirred at −72° C. for 1 hour. After this time, the reaction mixture was quenched with H₂O (200 mL) and extracted with ethyl acetate (100 mL×3). The combined organic extracts were washed with brine, dried over anhydrous Na₂SO₄, filtered, and concentrated to dryness under reduced pressure to afford the product, which was purified by FCC (eluent: petroleum ether:ethyl acetate=1:0 to 1:3) to provide the title compound (5.8 g, 30%) as a yellow solid. LC-MS (ESI): mass calcd. for C₁₁H₁₂INO₂ 316.99 m/z, found 318.0 [M+H]⁺. ¹H NMR (400 MHz, CDCl₃) δ 7.72-7.65 (m, 1H), 7.61-7.53 (m, 1H), 7.28-7.21 (m, 1H), 7.06-6.99 (m, 1H), 3.95-3.87 (m, 1H), 3.45-3.36 (m, 1H), 3.35-3.25 (m, 1H), 2.97-2.91 (m, 3H), 2.45-2.34 (m, 1H), 2.32-2.24 (m, 1H).

Step B. 3-(3-(3-Bromo-1H-1,2,4-triazol-1-yl)phenyl)-3-hydroxy-1-methylpyrrolidin-2-one. 3-Hydroxy-3-(3-iodophenyl)-1-methylpyrrolidin-2-one (3.0 g, 9.5 mmol), K₃PO₄ (4.2 g, 20 mmol), CuI (90 mg, 0.47 mmol), 3-bromo-1H-1,2,4-triazole (1.68 g, 11.4 mmol), N₁,N₂-dimethylcyclohexane-1,2-diamine (0.15 mL, 0.95 mmol) and DMF (20 mL) were added to a 100 mL flask. The resultant mixture was heated at 110° C. for 20 hours. After this time, the mixture was cooled to room temperature, filtered through a pad of diatomaceous earth and the pad was washed with ethyl acetate (80 mL). The filtrate was diluted with H₂O (30 mL). The two phases were separated, and the aqueous phase extracted with ethyl acetate (80 mL×3). The combined organic extracts were washed with brine, dried over anhydrous Na₂SO₄, filtered, and concentrated under reduced pressure to give the product, which was purified by FCC (eluent: petroleum ether:ethyl acetate=1:0 to 0:1, then ethyl acetate:methanol=1:0 to 10:1) to afford the title compound (1.3 g, 35%) as a yellow solid. LCMS (ESI), mass calcd. for C₁₃H₁₃BrN₄O₂ 336.02 m/z, found 336.8 [M+H]⁺.

Step C. 3-Hydroxy-1-methyl-3-(3-(3-(1-tosyl-1H-pyrrolo[2,3-b]pyridin-3-yl)-1H-1,2,4-triazol-1-yl)phenyl)pyrrolidin-2-one. 3-(3-(3-Bromo-1H-1,2,4-triazol-1-yl)phenyl)-3-hydroxy-1-methylpyrrolidin-2-one (500 mg, 1.48 mmol) was added to a mixture of 3 (4,4,5,5-tetramethyl-1,3,2-dioxaborolan-2-yl)-1-tosyl-1H-pyrrolo[2,3-b]pyridine (591 mg, 1.48 mmol), K₃PO₄ (944 mg, 4.45 mmol) and 1,4-dioxane/H₂O (4/1, 10 mL). The resultant mixture was sparged with N₂ for 5 minutes and then treated with Pd(dtbpf)Cl₂ (97 mg, 0.15 mmol). The resultant mixture was sparged with N₂ for another 5 minutes and then heated at 90° C. for 1 hour. After this time, the mixture was cooled to room temperature, filtered through a pad of diatomaceous earth and the pad was washed with ethyl acetate (30 mL). The filtrate was washed with brine, dried over anhydrous Na₂SO₄, filtered, and concentrated to give the product, which was purified by FCC (eluent: petroleum ether:ethyl acetate=1:0 to 0:1, then ethyl acetate:methanol=1:0 to 10:1) to afford the title compound (460 mg, 48%) as a pink solid. LCMS (ESI): mass calcd. For C₂₇H₂₄N₆O₄S 528.16 m/z, found 529.1 [M+H]⁺.

### Intermediate 179. 3-Hydroxy-1-methyl-3-(3-(3-(1-tosyl-1H-pyrrolo[2,3-b]pyridin-3-yl)-1H-pyrazol-1-yl)phenyl)pyrrolidin-2-one

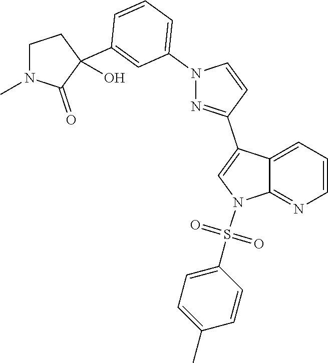 [SMILES: Cc1ccc(S(=O)(=O)n2cc(-c3ccn(-c4cccc(C5(O)CCN(C)C5=O)c4)n3)c3cccnc32)cc1]

Step A. 3-(1H-Pyrazol-3-yl)-1-tosyl-1H-pyrrolo[2,3-b]pyridine. 3-(4,4,5,5-Tetramethyl-1,3,2-dioxaborolan-2-yl)-1-tosyl-1H-pyrrolo[2,3-b]pyridine (5.00 g, 12.6 mmol), 3-bromo-1H-pyrazole (2.21 g, 15.1 mmol), and K₃PO₄ (7.99 g, 37.7 mmol) were added to a 100 mL flask and the resulting mixture dissolved in 1,4-dioxane (32 mL) and H₂O (8 mL). The mixture was sparged with Ar for 5 minutes and then treated with Pd(dtbpf)Cl₂ (818 mg, 1.26 mmol). The resultant mixture was sparged with Ar for another minutes and then heated at 100° C. for 3 hours. After this time, the mixture was cooled to room temperature and concentrated to dryness under reduced pressure to give the product, which was purified by FCC (eluent: petroleum ether:ethyl acetate=1:0 to 1:1) to give the title compound (1.5 g, 34%) as a yellow solid. LC-MS (ESI): mass calcd. For C₁₇H₁₄N₄O₂S 338.08 m/z found 338.4 [M+H ¹H NMR (400 MHz, DMSO-d₆) δ 12.98 (br s, 1H), 8.65-8.53 (m, 1H), 8.47-8.36 (m, 1H), 8.30 (s, 1H), 8.04-7.97 (m, 2H), 7.88-7.79 (m, 1H), 7.47-7.32 (m, 3H), 6.93-6.82 (m, 1H), 2.33 (s, 3H).

Step B. 3-Hydroxy-1-methyl-3-(3-(3-(1-tosyl-1H-pyrrolo[2,3-b]pyridin-3-yl)-1H-pyrazol-1-yl)phenyl)pyrrolidin-2-one. 3-(1H-Pyrazol-3-yl)-1-tosyl-1H-pyrrolo[2,3-b]pyridine (1.4 g, 4.1 mmol), 3-hydroxy-3-(3-iodophenyl)-1-methylpyrrolidin-2-one (Intermediate 178, Step A, 1.6 g, 5.0 mmol), Cs₂CO₃ (2.7 g, 8.3 mmol), and DMF (15 mL) were added to a 50 mL flask. The mixture was sparged with N₂ for 5 minutes and then treated with CuI (788 mg, 4.14 mmol) and 1,10-phenanthroline (746 mg, 4.14 mmol). The mixture was sparged with N₂ for another 5 minutes and then heated at 100° C. for 3 hours. After this time, the mixture was cooled to room temperature, filtered through a pad of diatomaceous earth, and the pad was washed with ethyl acetate (40 mL). The filtrate was concentrated to dryness under reduced pressure to afford the product, which was purified by FCC (eluent: petroleum ether:ethyl acetate=1:0 to 0:1) to give the title compound (355 mg, 16%) as a yellow solid. LC-MS (ESI): mass calcd. For C₂₈H₂₅N₅O₄S 527.16 m/z found 528.1 [M+H]⁺.

### Intermediate 180. 3-Hydroxy-1-methyl-3-(3-(4-(1-tosyl-1H-pyrrolo[2,3-b]pyridin-3-yl)-1H-1,2,3-triazol-1-yl)phenyl)pyrrolidin-2-one

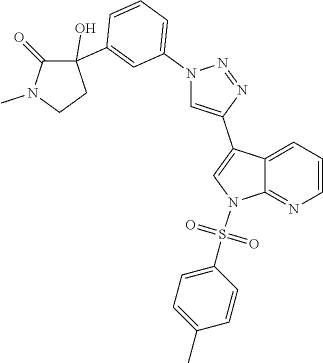 [SMILES: Cc1ccc(S(=O)(=O)n2cc(-c3cn(-c4cccc(C5(O)CCN(C)C5=O)c4)nn3)c3cccnc32)cc1]

Step A. 3-Bromo-1-tosyl-1H-pyrrolo[2,3-b]pyridine. 4-Toluenesulfonyl chloride (2.32 g, 12.2 mmol) was added to a solution of 3-bromo-1H-pyrrolo[2,3-b]pyridine (2.0 g, 10 mmol), DMAP (248 mg, 2.03 mmol), Et₃N (4.23 ml, 30.5 mmol), and methylene chloride (30 mL). The resultant mixture was stirred at room temperature for 16 hours. After this time, the reaction mixture was diluted with H₂O (50 mL) and extracted with methylene chloride (50 mL×3). The combined organic extracts were dried over anhydrous Na₂SO₄, filtered, and concentrated to dryness under reduced pressure to afford the product, which was purified by FCC (eluent: petroleum ether:ethyl acetate=1:0 to 2:1) to give the title compound (3.48 g, 92%) as a white solid. LC-MS (ESI): mass calcd. for C₁₄H₁₁BrN₂O₂S 349.97 m/z, found 351.0 [M+H]⁺.

Step B. 1-Tosyl-3-((trimethylsilyl)ethynyl)-1H-pyrrolo[2,3-b]pyridine. 3-Bromo-1-tosyl-1H-pyrrolo[2,3-b]pyridine (1.0 g, 2.8 mmol), trimethylsilylacetylene (0.60 mL, 4.3 mmol), Et₃N (5 mL), and DMF (5 mL) were added to a 50 mL flask. The mixture was sparged with Ar for 5 minutes and then treated with CuI (108 mg, 0.569 mmol) and Pd(PPh₃)₂Cl₂ (400 mg, 0.569 mmol). The resultant mixture was sparged with Ar for another 5 minutes and then heated at 80° C. for 16 hours. After this time, the mixture was cooled to room temperature and concentrated to dryness under reduced pressure to afford the product, which was purified by FCC (eluent: petroleum ether:ethyl acetate=1:0 to 5:1) to afford the title compound (835 mg, 79%) as a yellow solid. LCMS (ESI): mass calcd. for C₁₉H₂₀N₂O₂SSi 368.10 m/z, found 369.1 [M+H]⁺.

Step C. 3-(3-Azidophenyl)-3-hydroxy-1-methylpyrrolidin-2-one. Sodium azide (460 mg, 7.08 mmol) was added to a solution of 3-hydroxy-3-(3-iodophenyl)-1-methylpyrrolidin-2-one (Intermediate 178, Step A, 1.0 g, 3.2 mmol), DBU (0.141 mL, 0.946 mmol), and DMSO (10 mL). The mixture was sparged with Ar for 5 minutes and then treated with CuI (120 mg, 0.630 mmol). The resultant mixture was sparged with Ar for another 5 minutes and then heated at 95° C. for 3 hours. After this time, the mixture was cooled to room temperature and used to the next step directly.

Step D. 3-Hydroxy-1-methyl-3-(3-(4-(1-tosyl-1H-pyrrolo[2,3-b]pyridin-3-yl)-1H-1,2,3-triazol-1-yl)phenyl)pyrrolidin-2-one. 1-Tosyl-3-((trimethylsilyl)ethynyl)-1H-pyrrolo[2,3-b]pyridine (1.39 g, 3.78 mmol) was added to a solution of 3-(3-azidophenyl)-3-hydroxy-1-methylpyrrolidin-2-one and DMSO (10 mL). The mixture was stirred at room temperature for 2 hours. After this time, the pH of the reaction mixture was adjusted to pH>9 with NaOH (1.0 M in H₂O) and then extracted with methylene chloride (30 mL×2). The combined organic extracts were dried over anhydrous Na₂SO₄, filtered, and concentrated to dryness under reduced pressure to afford the product, which was purified by FCC (eluent: petroleum ether:ethyl acetate=1:0 to 0:1) to afford the title compound (60 mg, 2%) as a yellow solid. LCMS (ESI), mass calcd. for C₂₇H₂₄N₆O₄S 528.16 m/z, found 529.1 [M+H]⁺.

### Intermediate 181. 3-Hydroxy-1-methyl-3-(3-(4-(1-tosyl-1H-pyrrolo[2,3-b]pyridin-3-yl)-1H-imidazol-1-yl)phenyl)pyrrolidin-2-one

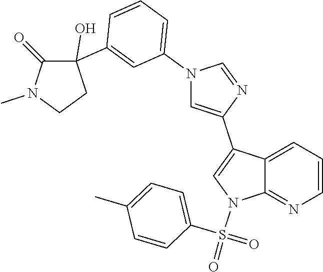 [SMILES: Cc1ccc(S(=O)(=O)n2cc(-c3cn(-c4cccc(C5(O)CCN(C)C5=O)c4)cn3)c3cccnc32)cc1]

Step A. 3-(3-(4-Bromo-1H-imidazol-1-yl)phenyl)-3-hydroxy-1-methylpyrrolidin-2-one. 4-Bromo-1H-imidazole (1.0 g, 6.8 mmol) was added to a solution of 3-hydroxy-3-(3-iodophenyl)-1-methylpyrrolidin-2-one (Intermediate 178, Step A, 2.2 g, 6.8 mmol), CS₂CO₃ (4.4 g, 14 mmol), and DMF (12 mL). The mixture was sparged with N₂ for 5 minutes and then treated with CuI (648 mg, 3.40 mmol). The mixture was sparged with N₂ for another 5 minutes and then stirred at 100° C. for 2 hours. After this time, the mixture was cooled to room temperature, diluted with water (15 mL), and extracted with ethyl acetate (20 mL×3). The combined organic extracts were washed with brine (20 mL), dried over anhydrous Na₂SO₄, filtered, and concentrated under reduced pressure to give the product, which was purified by FCC (eluent: petroleum ether:ethyl acetate=1:0 to 0:1) to afford the title compound (1.16 g, 50%) as a yellow solid. LCMS (ESI): mass calcd. for C₁₄H₁₄BrN₃O₂ 336.18 m/z, found 337.7 [M+H]⁺.

Step B. 3-Hydroxy-1-methyl-3-(3-(4-(1-tosyl-1H-pyrrolo[2,3-b]pyridin-3-yl)-1H-imidazol-1-yl)phenyl)pyrrolidin-2-one. Bis(triphenylphosphine)palladium(II) dichloride (239 mg, 0.327 mmol) was added to a solution of 3-(3-(4-bromo-1H-imidazol-1-yl)phenyl)-3-hydroxy-1-methylpyrrolidin-2-one (1.1 g, 3.3 mmol), 3-(4,4,5,5-tetramethyl-1,3,2-dioxaborolan-2-yl)-1-tosyl-1H-pyrrolo[2,3-b]pyridine (1.6 g, 3.9 mmol), K₃PO₄ (2.1 g, 9.8 mmol), and 1,4-dioxane/H₂O (15 mL, v/v=4:1). The resultant mixture was heated at 80° C. 3 hours. After this time, the mixture was cooled to room temperature, diluted with water (20 mL), and extracted with ethyl acetate (20 mL×3). The combined organic extracts were washed with brine (50 mL), dried over anhydrous Na₂SO₄, filtered, and concentrated under reduced pressure to give the product, which was purified by FCC (eluent: petroleum ether:ethyl acetate=1:0 to 0:1) to afford the title compound (1.2 g, 64%) as a brown solid. LCMS (ESI): mass calcd. for C₂₈H₂₅N₅O₂S 527.16 m/z, found 528.1 [M+H]⁺.

### Intermediate 182. (R)-3-Hydroxy-1-methyl-3-(3-(1-methyl-5-(1-tosyl-1H-pyrrolo[2,3-b]pyridin-3-yl)-1H-imidazol-2-yl)phenyl)pyrrolidin-2-one

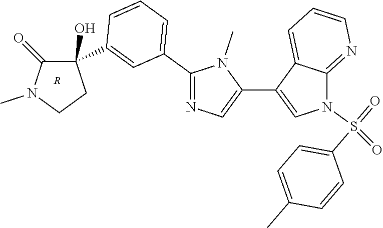

Step A. 3-(2-Bromo-1-methyl-1H-imidazol-5-yl)-1-tosyl-1H-pyrrolo[2,3-b]pyridine. To a microwave vial containing 3-(4,4,5,5-tetramethyl-1,3,2-dioxaborolan-2-yl)-1-tosyl-1H-pyrrolo[2,3-b]pyridine (1.0 g, 2.5 mmol), 2,5-dibromo-1-methyl-1H-imidazole (723 mg, 3.01 mmol) and K₃PO₄ (1.6 g, 7.5 mmol) was added Pd(dtbpf)Cl₂ (164 mg, 0.251 mmol) and 1,4-dioxane/H₂O (10 mL, v/v=4:1). The resultant mixture was heated at 80° C. via microwave irradiation for 1 hour. After this time, the mixture was cooled to room temperature, diluted with water (15 mL), and extracted with ethyl acetate (20 mL×3). The combined organic extracts were washed with brine (20 mL), dried over anhydrous Na₂SO₄, filtered, and concentrated under reduced pressure to give the product, which was purified by FCC (eluent: petroleum ether:ethyl acetate=1:0 to 1:1) to afford the title compound (265 mg, 23%) as a colorless oil. LCMS (ESI): mass calcd. for C₁₈H₁₅BrN₄O₂S 430.01 m/z, found 431.0 [M+H]⁺. ¹H NMR (400 MHz, CDCl₃) δ 8.46-8.41 (m, 1H), 8.07 (d, J=8.4 Hz, 2H), 7.79-7.74 (m, 1H), 7.71 (s, 1H), 7.25 (d, J=8.2 Hz, 2H), 7.18-7.16 (m, 1H), 7.08 (s, 1H), 3.53 (s, 3H), 2.33 (s, 3H).

Step B. (R)-3-Hydroxy-1-methyl-3-(3-(1-methyl-5-(1-tosyl-1H-pyrrolo[2,3-b]pyridin-3-yl)-1H-imidazol-2-yl)phenyl)pyrrolidin-2-one. To a microwave vial containing 3-(2-bromo-1-methyl-1H-imidazol-5-yl)-1-tosyl-1H-pyrrolo[2,3-b]pyridine (101 mg, 0.234 mmol), (R)-3-hydroxy-1-methyl-3-(3-(4,4,5,5-tetramethyl-1,3,2-dioxaborolan-2-yl)phenyl)pyrrolidin-2-one (Intermediate 16, 74 mg, 0.23 mmol), K₃PO₄ (149 mg, 0.703 mmol) and Xphos-Pd-G₂ (18 mg, 0.023 mmol) was added 1,4-dioxane/H₂O (5 mL, v/v=4:1). The resultant mixture was sparged with Ar for 5 minutes and then heated at 100° C. via microwave irradiation for 1 hour. After this time, the mixture was cooled to room temperature and concentrated under reduced pressure to afford the product, which was purified by FCC (eluent: ethyl acetate:methanol=1:0 to 9:1) to afford the title compound (95 mg, 58%) as a yellow solid. LCMS (ESI): mass calcd. for C₂₉H₂₇N₅O₄S 541.62 m/z, found 542.1 [M+H]⁺.

### Intermediate 183. (R)-3-(3-(2-(2,5-Dimethyl-1-tosyl-1H-pyrrolo[2,3-b]pyridin-3-yl)thiazol-4-yl)phenyl)-3-hydroxy-1-methylpyrrolidin-2-one

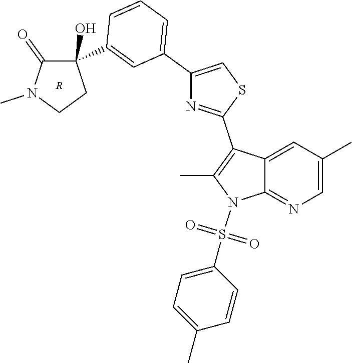

Step A. 5-Bromo-2-methyl-1-tosyl-1H-pyrrolo[2,3-b]pyridine. TsCl (3.39 g, 17.8 mmol) was added to a solution at 0° C. of 5-bromo-2-methyl-1H-pyrrolo[2,3-b]pyridine (2.5 g, 12 mmol), powdered sodium hydroxide (1.42 g, 35.5 mmol), benzyltriethylammonium chloride (270 mg, 1.19 mmol), and dichloromethane (30 mL). The resultant mixture was stirred for 16 hours with gradual warming to room temperature. The suspension was filtered, and the filtrate was concentrated to dryness under reduced pressure to afford the product, which was triturated with petroleum ether:ethyl acetate (5:1, 30 mL) and the solids were isolated via filtration. The filter cake was washed with petroleum ether (20 mL) and dried in vacuo to afford the title compound (3.2 g, 70%) as a brown solid. LCMS (ESI): mass calcd. for C₁₅H₁₃BrN₂O₂S 363.99 m/z, found 365.0 [M+H]⁺.

Step B. 2,5-Dimethyl-1-tosyl-1H-pyrrolo[2,3-b]pyridine. 5-Bromo-2-methyl-1-tosyl-1H-pyrrolo[2,3-b]pyridine (3.2 g, 8.8 mmol), potassium trifluoro(methyl)borate (2.1 g, 17 mmol), K₃PO₄ (4.6 g, 22 mmol), 1,4-dioxane (45 mL), and H₂O (15 mL) were added to a 100 mL round-bottomed flask. The resultant mixture was sparged with Ar for 5 minutes and then treated with Pd(dppf)Cl₂ (641 mg, 0.876 mmol). The mixture was heated at 110° C. for 16 hours. The reaction mixture was cooled to room temperature quenched with water (80 mL) and extracted with ethyl acetate (50 mL×3). The combined organic extracts were dried over anhydrous Na₂SO₄, filtered, and concentrated to dryness under reduced pressure to give the product, which was purified by silica gel chromatography (petroleum ether:ethyl acetate=20:1 to 5:1) to afford the title compound (1.1 g, 29%) as a white solid. LCMS (ESI): mass calcd. for C₁₆H₁₆N₂O₂S 300.09 m/z, found 301.1 [M+H]⁺.

Step C. 3-Bromo-2,5-dimethyl-1-tosyl-1H-pyrrolo[2,3-b]pyridine. NBS (652 mg, 3.66 mmol) was added to a 0° C. solution of 2,5-dimethyl-1-tosyl-1H-pyrrolo[2,3-b]pyridine (1.0 g, 3.3 mmol) and dichloromethane (20 mL). The resultant mixture was stirred for 16 hours with gradual warming to room temperature. The reaction mixture was quenched with water (30 mL) and extracted with dichloromethane (30 mL×3). The combined organic extracts were dried over anhydrous Na₂SO₄, filtered, and concentrated to dryness in vacuo to give the product, which was initially purified by silica gel chromatography (petroleum ether:ethyl acetate=20:1 to 5:1). The title compound was further purified by preparative HPLC using a YMC-Triart Prep C18 150×40 mm×7 μm column (eluent: 70% to 80% (v/v) CH₃CN and H₂O with 0.04% NH₃H₂O+10 mM NH₄HCO₃). The product was suspended in water (10 mL), the mixture frozen using dry ice/acetone, and then lyophilized to dryness to afford the title compound (520 mg, 41%) as a white solid. LCMS (ESI): mass calcd. for C₁₆H₁₅BrN₂O₂S 378.00 m/z, found 379.0 [M+H]⁺.

Step D. 2,5-Dimethyl-3-(4,4,5,5-tetramethyl-1,3,2-dioxaborolan-2-yl)-1-tosyl-1H-pyrrolo[2,3-b]pyridine. To a microwave vial containing 3-bromo-2,5-dimethyl-1-tosyl-1H-pyrrolo[2,3-b]pyridine (480 mg, 1.27 mmol), 4,4,4′,4′,5,5,5′,5′-octamethyl-2,2′-bi(1,3,2-dioxaborolane) (643 mg, 2.53 mmol), K₂CO₃ (437 mg, 3.16 mmol) and tricyclohexylphosphine (53 mg, 0.19 mmol) was added 1,4-dioxane (8 mL). The mixture was sparged with N₂ for 5 minutes and then treated with Pd₂(dba)₃ (116 mg, 0.13 mmol). The mixture was sparged with N₂ for another 5 min and then heated at 100° C. via microwave irradiation for 1 hour. The contents were cooled to room temperature, the suspension was filtered and the filtrate was concentrated to dryness in vacuo to afford the product, which was purified by silica gel chromatography (eluent: petroleum ether:ethyl acetate=20:1 to 3:1) to afford the title compound (360 mg, 43%) as a brown solid. LCMS (ESI): mass calcd. for C₂₂H₂₇BN₂O₄S 426.18 m/z, found 427.2 [M+H]⁺.

Step E. 4-Bromo-2-(2,5-dimethyl-1-tosyl-1H-pyrrolo[2,3-b]pyridin-3-yl)thiazole. To a microwave vial containing 2,5-dimethyl-3-(4,4,5,5-tetramethyl-1,3,2-dioxaborolan-2-yl)-1-tosyl-1H-pyrrolo[2,3-b]pyridine (350 mg, 0.821 mmol), 2,4-dibromothiazole (299 mg, 1.23 mmol) and Cs₂CO₃ (669 mg, 2.05 mmol) was added 1,4-dioxane (10 mL), and H₂O (2 mL). The mixture was sparged with Ar for 5 minutes and then treated with Pd(dppf)Cl₂ (60 mg, 0.08 mmol). The mixture was sparged with Ar for another 5 minutes and the resultant mixture was heated at 80° C. via microwave irradiation for 1 hour. After this time, the mixture was cooled to room temperature, diluted with water (50 mL), and extracted with ethyl acetate (50 mL×3). The combined organic extracts were dried over anhydrous Na₂SO₄, filtered and concentrated to dryness in vacuo to give the product, which was purified by silica gel chromatography (eluent: petroleum ether:ethyl acetate=20:1 to 3:1) to afford the title compound (250 mg, 53%) as a brown solid. LCMS (ESI): mass calcd. for C₁₉H₁₆BrN₃O₂S₂ 460.99 m/z, found 462.0 [M+H]⁺.

Step F. (R)-3-(3-(2-(2,5-Dimethyl-1-tosyl-1H-pyrrolo[2,3-b]pyridin-3-yl)thiazol-4-yl)phenyl)-3-hydroxy-1-methylpyrrolidin-2-one. To a microwave vial containing 4-bromo-2-(2,5-dimethyl-1-tosyl-1H-pyrrolo[2,3-b]pyridin-3-yl)thiazole (230 mg, 0.497 mmol), (R)-3-hydroxy-1-methyl-3-(3-(4,4, 5,5-tetramethyl-1,3,2-dioxaborolan-2-yl)phenyl)pyrrolidin-2-one (Intermediate 16, 126 mg, 0.397 mmol) and K₃PO₄ (317 mg, 1.49 mmol) was added 1,4-dioxane (10 mL), and H₂O (2 mL). The mixture was sparged with Ar for 5 minutes and then treated with Pd(dtbpf)Cl₂ (32 mg, 0.05 mmol). The mixture was sparged with Ar for another 5 minutes and the mixture was heated at 80° C. via microwave irradiation for 1 hour. The reaction mixture was then cooled to room temperature, diluted with water (50 mL) and extracted with ethyl acetate (50 mL×3). The combined organic extracts were dried over anhydrous Na₂SO₄, filtered, and concentrated to dryness in vacuo to give the product, which was purified by silica gel chromatography (eluent: petroleum ether:ethyl acetate=10:1 to 0:1) to afford the title compound (120 mg, 41%) as a brown solid. LCMS (ESI): mass calcd. for C₃₀H₂₈N₄O₄S₂ 572.16 m/z, found 573.2 [M+H]⁺.

### Intermediate 184. 3-Hydroxy-1-methyl-3-(3-(4-(1-tosyl-1H-pyrrolo[2,3-b]pyridin-3-yl)-2H-1,2,3-triazol-2-yl)phenyl)pyrrolidin-2-one

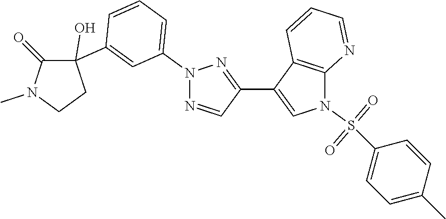

Step A. 3-(3-(4-Bromo-2H-1,2,3-triazol-2-yl)phenyl)-3-hydroxy-1-methylpyrrolidin-2-one. 4-Bromo-1H-1,2,3-triazole (1.0 g, 6.8 mmol), 3-hydroxy-3-(3-iodophenyl)-1-methylpyrrolidin-2-one (Intermediate 178, Step A, 2.57 g, 8.11 mmol), Cs₂CO₃ (4.40 g, 13.5 mmol) and DMA (20 mL) were added to a 50 mL flask. The mixture was sparged with N₂ three times and then treated with CuI (257 mg, 1.35 mmol) and 2 (dimethylamino)acetic acid (697 mg, 6.76 mmol). The mixture was sparged with N₂ another three times and then heated at 130° C. for 3 hours. After this time, the mixture was cooled to room temperature and concentrated to dryness under reduced pressure to afford the product, which was purified by FCC (eluent: petroleum ether:ethyl acetate=1:0 to 1:3) to give the title compound (1.6 g, 27%) as a yellow oil. LC-MS (ESI): mass calcd. for C₁₃H₁₃BrN₄O₂ 336.02 m/z found 337.0 [M+H]⁺.

Step B. 3-Hydroxy-1-methyl-3-(3-(4-(1-tosyl-1H-pyrrolo[2,3-b]pyridin-3-yl)-2H-1,2,3-triazol-2-yl)phenyl)pyrrolidin-2-one. 3-(4,4, 5,5-Tetramethyl-1,3,2-dioxaborolan-2-yl)-1-tosyl-1H-pyrrolo[2,3-b]pyridine (1.43 g, 3.59 mmol), 3-(3-(4-bromo-2H-1,2,3-triazol-2-yl)phenyl)-3-hydroxy-1-methylpyrrolidin-2-one (2.2 g, 1.8 mmol), and K₃PO₄ (1.14 g, 5.38 mmol) were added to a 100 mL flask and the resulting mixture dissolved in 1,4-dioxane (16 mL) and H₂O (4 mL). The mixture was sparged with Ar three times, treated with Pd(dppf)Cl₂ (131 mg, 0.179 mmol), sparged with Ar an additional three times, and then heated at 80° C. for 3 hours. After this time, the mixture was cooled to room temperature and concentrated to dryness under reduced pressure to give the product, which was purified with preparative HPLC over a Phenomenex Genimi NX C18 150×40 mm×5 um column (eluent: 38% to 68% (v/v) ACN and H₂O with 0.05% NH₃·H₂O and 10 mM NH₄HCO₃). The pure fractions were combined and the solvents were removed under vacuum. The residue was re-suspended in water (20 mL) and the resulting mixture was lyophilized to dryness to remove the solvent residue completely to give title compound (360 mg, 34%) as a white solid. LC-MS (ESI): mass calcd. for C₂₇H₂₄N₆O₄S 528.16 m/z found 529.1 [M+H]⁺. ¹H NMR (400 MHz, DMSO-d₆) δ 8.78 (s, 1H), 8.73 (s, 1H), 8.70-8.67 (m, 1H), 8.51-8.48 (m, 1H), 8.21-8.19 (m, 1H), 8.08-8.04 (m, 3H), 7.59-7.53 (m, 1H), 7.51-7.43 (m, 3H), 7.38-7.34 (m, 1H), 6.29 (s, 1H), 3.52-3.45 (m, 1H), 3.43-3.36 (m, 1H), 2.87 (s, 3H), 2.42-2.36 (m, 1H), 2.35 (s, 3H), 2.33-2.26 (m, 1H).

### Intermediate 185. (R)-3-hydroxy-1-methyl-3-(3-(2-(5-methyl-1-tosyl-1H-pyrrolo[3,2-b]pyridin-3-yl)thiazol-4-yl)phenyl)pyrrolidin-2-one

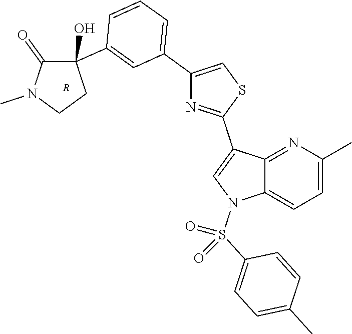

Step A. 3-Bromo-5-methyl-1H-pyrrolo[3,2-b]pyridine. Bromopyrrolidine-2,5-dione (675 mg, 3.79 mmol) was added to a mixture of 5-methyl-1H-pyrrolo[3,2-b]pyridine (500 mg, 3.78 mmol) and DMF (20 mL). The resultant mixture was stirred at room temperature for 1 hour. After this time, the mixture was quenched with sat. Na₂S₂O₃ (10 mL), diluted with water (40 mL), and extracted with ethyl acetate (30 mL×3). The combined organic extracts were washed with brine, dried over anhydrous Na₂SO₄, filtered, and concentrated to dryness under reduced pressure to afford the product, which was purified by FCC (eluent: petroleum ether:ethyl acetate=1:0 to 1:1) to give the title compound (0.8 g, 99%) as a yellow solid. LCMS (ESI): mass calcd. for C₈H₇BrN₂ 209.98 m/z, found 210.7 [M+H]⁺.

Step B. 3-Bromo-5-methyl-1-tosyl-1H-pyrrolo[3,2-b]pyridine. A solution of 3-bromo-5-methyl-1H-pyrrolo[3,2-b]pyridine (1.3 g, 6.2 mmol) and THF (5 mL) was added dropwise to a 0° C. mixture of NaH (368 mg, 60 wt. % in mineral oil, 9.20 mmol) and THF (16 mL) under a N₂ atmosphere. The resultant mixture was stirred at 0° C. for 30 minutes, and then treated with a solution of TosCl (1.8 g, 9.4 mmol) in THF (5 mL), and stirred 16 hours with gradual warming to room temperature. After this time, the mixture was diluted with water (30 mL) and extracted with ethyl acetate (30 mL×3). The combined organic extracts were washed with brine, dried over anhydrous Na₂SO₄, filtered, and concentrated to dryness under reduced pressure to give the product, which was purified by FCC (eluent: petroleum ether:ethyl acetate=1:0 to 3:1) to afford the title compound (1.95 g, 74%) as a yellow solid. LCMS (ESI): mass calcd. For C₁₅H₁₃BrN₂O₂S 363.99 m/z, found 364.7 [M+H]⁺.

Step C. (5-Methyl-1-tosyl-1H-pyrrolo[3,2-b]pyridin-3-yl)boronic acid. n-Butyllithium (5.2 mL, 13 mmol, 2.5 M in hexane) was added dropwise to a −72° C. solution of 3-bromo-1-tosyl-1H-pyrrolo[2,3-b]pyridine (2.15 g, 5.89 mmol), triisopropyl borate (2.8 mL, 12 mmol), and THE (30 mL). The resultant mixture was stirred at −72° C. for 30 minutes. After this time, the mixture was quenched with water (30 mL) and extracted with ethyl acetate (50 mL×3). The combined organic extracts were washed with brine, dried over anhydrous Na₂SO₄, filtered, and concentrated under reduced pressure to give the title compound (2.0 g, 52% purity, 54%) as a yellow solid. LCMS (ESI): mass calcd. For C₁₅H₁₅BN₂O₄S 330.08 m/z, found 330.9 [M+H]⁺.

Step D. 4-Bromo-2-(5-methyl-1-tosyl-1H-pyrrolo[3,2-b]pyridin-3-yl)thiazole. To a microwave vial containing (5-methyl-1-tosyl-1H-pyrrolo[3,2-b]pyridin-3-yl)boronic acid (400 mg, 1.21 mmol), 2,4-dibromothiazole (412 mg, 1.70 mmol) and K₃PO₄ (772 mg, 3.64 mmol) was added 1,4-dioxane/H₂O (10 mL, v/v=4:1). The resultant mixture was sparged with N₂ for 5 minutes and then treated with Pd(dtbpf)Cl₂ (80 mg, 0.12 mmol). The mixture was sparged with N₂ for another 5 minutes and then heated at 90° C. via microwave irradiation for 1 hour. After this time, the mixture was cooled to room temperature, diluted with water H₂O (10 mL), and extracted with ethyl acetate (20 mL×3). The combined organic extracts were washed with brine, dried over anhydrous Na₂SO₄, filtered, and concentrated to dryness under reduced pressure to give the product, which was purified by FCC (eluent: petroleum ether:ethyl acetate=1:0 to 3:1) to afford the title compound (140 mg, 24%) as a white solid. LCMS (ESI): mass calcd. For C₁₈H₁₄BrN₃O₂S₂ 446.97 m/z, found 449.9 [M+H]⁺.

Step E. (R)-3-Hydroxy-1-methyl-3-(3-(2-(5-methyl-1-tosyl-1H-pyrrolo[3,2-b]pyridin-3-yl)thiazol-4-yl)phenyl)pyrrolidin-2-one. To a microwave vial containing 4-bromo-2-(5-methyl-1-tosyl-1H-pyrrolo[3,2-b]pyridin-3-yl)thiazole (300 mg, 0.669 mmol) (R)-3-hydroxy-1-methyl-3-(3-(4,4,5,5-tetramethyl-1,3,2-dioxaborolan-2-yl)phenyl)pyrrolidin-2-one (Intermediate 16, 210 mg, 0.662 mmol) and K₃PO₄ (420 mg, 1.98 mmol) was added 1,4-dioxane/H₂O (8 mL, v/v=4:1). The resultant mixture was sparged with N₂ for 5 minutes and then treated with Pd(dtbpf)Cl₂ (45 mg, 0.069 mmol). The mixture was purged with N₂ for another 5 minutes and then heated at 90° C. via microwave irradiation for 1 hour. After this time, the mixture was cooled to room temperature, diluted with water (10 mL), and extracted with ethyl acetate (30 mL×3). The combined organic extracts were washed with brine (10 mL), dried over anhydrous Na₂SO₄, filtered, and concentrated under reduced pressure to afford the title product (435 mg, 85% purity, 99%) as a yellow solid. LCMS (ESI): mass calcd. for C₂₉H₂₆N₄O₄S₂ 558.14 m/z, found 559.0 [M+H]⁺.

### Intermediate 186. 3-Methyl-6-(3-(2-(1-(phenylsulfonyl)-1H-pyrrolo[2,3-b]pyridin-3-yl)thiazol-4-yl)phenyl)-5,6-dihydro-4H-cyclopenta[d]isoxazol-6-ol

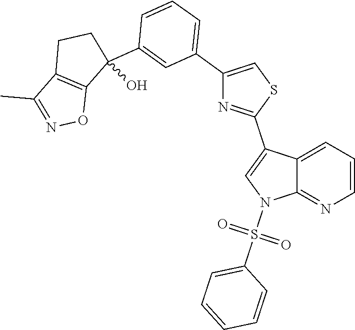 [SMILES: Cc1noc2c1CCC2(O)c1cccc(-c2csc(-c3cn(S(=O)(=O)c4ccccc4)c4ncccc34)n2)c1]

Step A. 2-Bromocyclopent-2-enone. N-Bromo-succinimide (21.7 g, 122 mmol) was added to a 0° C. mixture of cyclopent-2-enonel (10.0 g, 122 mmol), pyridine-N-oxide (17.4 g, 183 mmol), and ACN (600 mL). The mixture was stirred for 24 h with gradual warming to room temperature. After this time, the mixture was concentrated under reduced pressure to afford the product, which was purified by FCC (eluent: petroleum ether:EtOAC=1:0 to 4:1) to afford the title compound (14.67 g, 75%) as a yellow oil. LCMS (ESI): mass calcd. for C₅H₅BrO 159.95 m/z, found 161.1 [M+H]⁺.

Step B. 3-Methyl-4H-cyclopenta[d]isoxazol-6(5H)-one. N-Chloro-succinimide (9.73 g, 72.9 mmol) was added to a 0° C. mixture of acetaldehyde oxime (3.23 g, 54.7 mmol) and DMF (300 mL). The resultant mixture was stirred for 15 min at 0° C. and then stirred for 1.5 h with gradual warming to room temperature. The mixture was then treated with 2-bromocyclopent-2-enone (14.7 g, 91.1 mmol), followed by sodium bicarbonate (10.3 g, 123 mmol). After 16 h at room temperature, the mixture was poured into water (300 mL) and extracted with EtOAc (200 mL×3). The combined organic extracts were washed with brine (250 mL), dried over anhydrous Na₂SO₄, filtered and concentrated to dryness under reduced pressure to afford product, which was purified by FCC (eluent: petroleum ether:EtOAc=1:0 to 4:1) to afford the title compound (3.6 g, 78% purity, 22%) as a white solid. LCMS (ESI): mass calcd. for C₇H₇NO₂ 137.14 m/z, found 137.8 [M+H]⁺. ¹H NMR (400 MHz, CDCl₃) δ 3.13-3.07 (m, 2H), 2.80-2.73 (m, 2H), 2.33 (s, 3H).

Step C. 6-(3-Bromophenyl)-3-methyl-5,6-dihydro-4H-cyclopenta[d]isoxazol-6-ol. n-Butyllithium (2.5 M solution in hexane, 8.3 mL, 21 mmol) was added dropwise to a −78° C. solution of 1,3-dibromobenzene (2.7 mL, 22 mmol) in anhydrous THF (25 mL). The reaction mixture was stirred at −78° C. for 15 min, then, 3-methyl-4H-cyclopenta[d]isoxazol-6(5H)-one (3.0 g, 17 mmol) in anhydrous THF (5 mL) was added. The reaction mixture was stirred for 30 min at −78° C. and then quenched with water (30 mL) and extracted with EtOAc (20 mL×3). The combined organic extracts were washed with brine (50 mL), dried over anhydrous Na₂SO₄, filtered, and concentrated to dryness under reduced pressure to afford the product, which was purified by HPLC using a YMC-Triart Prep C18 150×40 mm×7 μm column (eluent: 50% to 60% (v/v) ACN and water (0.04% NH₃H₂O+10 mM NH₄HCO₃)) to give the title compound (1.52 g, 29%) as a red solid. LCMS (ESI): mass calcd. for C₁₃H₁₂BrNO₂ 293.00 m/z, found 294.0 [M+H]⁺. ¹H NMR (400 MHz, CDCl₃) δ 7.60-7.53 (m, 1H), 7.46-7.38 (m, 1H), 7.33-7.26 (m, 1H), 7.23-7.18 (m, 1H), 2.99-2.89 (m, 2H), 2.75-2.67 (m, 1H), 2.55-2.45 (m, 1H), 2.27 (s, 3H).

Step D. 6-(3-Bromophenyl)-3-methyl-6-((trimethylsilyl)oxy)-5,6-dihydro-4H-cyclopenta[d]isoxazole. Triethylamine (4.8 mL, 34 mmol) was added to the solution of 6-(3-bromophenyl)-3-methyl-5,6-dihydro-4H-cyclopenta[d]isoxazol-6-ol (1.0 g, 3.4 mmol) and DCM (25 mL). Then, the mixture was cooled to 0° C., and TMSCl (4.3 mL, 34 mmol, 0.856 g/mL) was added. The reaction mixture was heated to 40° C. for 16 hours, and then quenched with water (10 mL) and extracted with DCM (10 mL×3). The combined organic extracts were washed with brine (20 mL), dried over anhydrous Na₂SO₄, filtered, and concentrated to dryness under reduced pressure to afford the product, which was purified by FCC (petroleum ether:DCM=1:0 to 1:5) to give the title compound (958 mg, 67%) as a colorless oil. LCMS (ESI): mass calcd. for C₁₆H₂₀BrNO₂Si 365.04 m/z, found 366.0 [M+H]⁺.

Step E. (3-(3-Methyl-6-((trimethylsilyl)oxy)-5,6-dihydro-4H-cyclopenta[d]isoxazol-6-yl)phenyl)boronic acid. n-Butyllithium (2.5 M solution in hexane, 1.82 mL, 4.55 mmol) was added dropwise to a −78° C. solution of 6-(3-bromophenyl)-3-methyl-5,6-dihydro-4H-cyclopenta[d]isoxazol-6-ol (958 mg, 2.28 mmol) and triisopropyl borate (1.05 mL, 4.55 mmol) in THE (20 mL). The mixture was stirred at −78° C. for 30 min and then quenched with water (2 mL). After being warmed to room temperature, the mixture was concentrated under reduced pressure to afford the product (856 mg) as a brown oil. LCMS (ESI): mass calcd. for C₁₆H₂₂BNO₄Si 331.14 m/z, found 332.2 [M+H]⁺.

Step F. 3-Methyl-6-(3-(2-(1-(phenylsutfonyl)-1H-pyrrolo[2,3-b]pyridin-3-yl)thiazol-4-yl)phenyl)-5,6-dihydro-4H-cyclopenta[d]isoxazol-6-ol. To a microwave vial containing (3-(3-methyl-6-((trimethylsilyl)oxy)-5,6-dihydro-4H-cyclopenta[d]isoxazol-6-yl)phenyl)boronic acid (830 mg, 2.21 mmol), 4-bromo-2-(1-(phenylsulfonyl)-1H-pyrrolo[2,3-b]pyridin-3-yl)thiazole (Intermediate 2, 1.11 g, 2.65 mmol) and K₃PO₄ (1.40 g, 6.62 mmol) in 1,4-dioxane/H₂O=4:1 (15 mL) was added Pd(dtbpf)Cl₂ (144 mg, 0.221 mmol). The resultant mixture was heated for 1 hour at 100° C. via microwave irradiation. After this time, the mixture was diluted with water (20 mL) and extracted with EtOAc (20 mL×3). The combined organic extracts were washed with brine (30 mL), dried over anhydrous Na₂SO₄, filtered, and concentrated under reduced pressure to afford the product (1.38 g) as a white solid. LCMS (ESI): mass calcd. for C₂₉H₂₂N₄O₄S₂ 554.10 m/z, found 555.2 [M+H]⁺.

### Intermediate 187. (R)-3-(3-(2-bromothiazol-4-yl)phenyl)-3-hydroxy-1-methylpyrrolidin-2-one

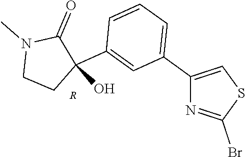

Step A. tert-butyl (4-(3-(3-Hydroxy-1-methyl-2-oxopyrrolidin-3-yl)phenyl)thiazol-2-yl)carbamate. 3-Hydroxy-1-methyl-3-(3-(4,4,5,5-tetramethyl-1,3,2-dioxaborolan-2-yl)phenyl)pyrrolidin-2-one (4.77 g, 15.0 mmol), tert-butyl (4-bromothiazol-2-yl)carbamate (4.2 g, 15 mmol), K₃PO₄ (6.4 g, 30 mmol), H₂O (10 mL), and 1,4-dioxane (50 mL) were added to a 100 mL three-necked round-bottomed flask. The resultant mixture was purged with nitrogen for 5 minutes and then treated with Pd(dtbpf)Cl₂ (981 mg, 1.51 mmol). The mixture was heated at 90° C. for 16 hours and then cooled to room temperature. The reaction mixture was then combined with another identical reaction mixture and the combined mixture was filtered. The filtrate was extracted with ethyl acetate (100 mL×3). The combined organic extracts were washed with brine, dried over anhydrous Na₂SO₄, filtered, and concentrated to dryness under reduced pressure to give the product, which was purified by FCC (eluent: petroleum ether:ethyl acetate=1:0 to 0:1) to afford the title compound. LCMS (ESI): mass calcd. for C₁₉H₂₃N₃O₄S, 389.47; m/z found, 390.2 [M+H]⁺.

Step B. 3-(3-(2-Aminothiazol-4-yl)phenyl)-3-hydroxy-1-methylpyrrolidin-2-one. tert-butyl (4-(3-(3-Hydroxy-1-methyl-2-oxopyrrolidin-3-yl)phenyl)thiazol-2-yl)carbamate (7.0 g, 18 mmol), TFA (50 mL), and dichloromethane (50 mL) were stirred for 2 hours at room-temperature. The mixture was concentrated to dryness under reduced pressure to give the product. The pH of the product was adjusted to pH 8 with Sat. NaHCO₃, the suspension was filtered, and the filter cake was washed with H₂O (10 mL×3) before drying under reduced pressure to afford the title compound (5.0 g) as a brown solid. LCMS (ESI): mass calcd. for C₁₄H₁₅N₃O₂S, 289.35; m/z found, 290.0 [M+H]⁺.

Step C. (R,S)-3-(3-(2-Bromothiazol-4-yl)phenyl)-3-hydroxy-1-methylpyrrolidin-2-one. t-BuONO (2.5 mL, 21 mmol) was added to a suspension of 3-(3-(2-aminothiazol-4-yl)phenyl)-3-hydroxy-1-methylpyrrolidin-2-one (2.0 g, 6.9 mmol) in acetonitrile (50 mL). Then, CuBr (3.0 g, 21 mmol) was added in portions. The reaction mixture was stirred for 16 hours at 30° C. The reaction mixture was combined with another identical reaction mixture and the combined mixture was filtered and concentrated to dryness under reduced pressure to afford the product, which was purified by FCC (eluent: petroleum ether:ethyl acetate=10:1 to 0:1) to afford the title compound (1.1 g) as a brown oil. LCMS (ESI): mass calcd. for C₁₄H₁₃BrN₂O₂S, 353.23; m/z found, 354.9 [M+H]⁺.

Step D. (R)-3-(3-(2-Bromothiazol-4-yl)phenyl)-3-hydroxy-1-methylpyrrolidin-2-one. (R,S)-3-(3-(2-bromothiazol-4-yl)phenyl)-3-hydroxy-1-methylpyrrolidin-2-one (2.2 g, crude) was purified by preparative HPLC using a YMC-Triart Prep C18, 250×50 mm×10 μm column (eluent: 10% to 54% (v/v) CH₃CN and H₂O with 0.225% HCOOH) to afford the pure product. The product was suspended in water (10 mL) and frozen using dry ice/acetone, and then lyophilized to dryness to afford the compound (750 mg) as a white solid, The (R) and (S) enantiomers of (R,S)-3-(3-(2-bromothiazol-4-yl)phenyl)-3-hydroxy-1-methylpyrrolidin-2-one were separated by SFC using a DAICEL CHIRALPAK AD, 250×30 mm×10 μm column (eluent: 45% to 45% (v/v) 0.1°% NH₃H₂O ETOH) to afford the first eluting enantiomer as (R)-3-(3-(2-Bromothiazol-4-yl)phenyl)-3-hydroxy-1-methylpyrrolidin-2-one (330 mg), as a brown solid. Rₜ=0.800 min, LCMS (ESI): mass calcd. for C₁₄H₁₃BrN₂O₂S, 353.23; m/z found, 354.9 [M+H]⁺. The second eluting enantiomer, (S)-3-(3-(2-Bromothiazol-4-yl)phenyl)-3-hydroxy-1-methylpyrrolidin-2-one, was also obtained (307.6 mg) as a brown solid. Rₜ=2.74 min. LCMS (ESI): mass calcd. for C₁₄H₁₃BrN₂O₂S, 353.23; m/z found, 354.9 [M+H]⁺.

### Example 1. (S)-7-(3-(2-(1H-Pyrrolo[2,3-b]pyridin-3-yl)thiazol-4-yl)phenyl)-6,7-dihydro-5H-cyclopenta[b]pyridin-7-ol

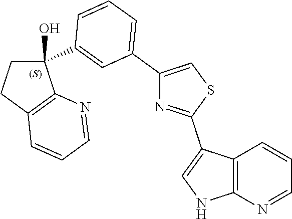

To a flask containing (R,S)-7-(3-(2-(1-(phenylsulfonyl)-1H-pyrrolo[2,3-b]pyridin-3-yl)thiazol-4-yl)phenyl)-6,7-dihydro-5H-cyclopenta[b]pyridin-7-ol (Intermediate 3, 300 mg, 0.55 mmol) and 1,4-dioxane (30 mL) was added LiOH (1.5 mL, 3 M in H₂O, 4.5 mmol). The resultant mixture was heated at 70° C. for 2 h. The mixture was cooled to room temperature, concentrated to dryness, and purified by preparative HPLC using an HPLC column, such as a Phenomenex Gemini 150×25 mm×10 μm column (eluent: 35% to 65%, CH₃CN and H₂O (with 0.04% NH₄OH+10 mM NH₄HCO₃)). The product was suspended in water (10 mL) and the mixture was frozen by insertion into a −78° C. bath. The frozen mixture was then lyophilized to yield (R,S)-(3-(2-(1H-pyrrolo[2,3-b]pyridin-3-yl)thiazol-4-yl)phenyl)-6,7-dihydro-5H-cyclopenta[b]pyridin-7-ol (87 mg, 38%) as a white solid. ¹H NMR (400 MHz, DMSO-d₆) δ 12.29 (br s, 1H), 8.51 (d, J=7.2 Hz, 1H), 8.42 (d, J=4.0 Hz, 1H), 8.36 (dd, J=4.4, 1.2 Hz, 1H), 8.26 (d, J=2.8 Hz, 1H), 8.00 (s, 1H), 7.92-7.87 (m, 2H), 7.80 (d, J=7.6 Hz, 1H), 7.44-7.38 (m, 1H), 7.37-7.33 (m, 1H), 7.33-7.26 (m, 2H), 5.91 (s, 1H), 3.16-3.06 (m, 1H), 2.99-2.88 (m, 1H), 2.48-2.38 (m, 2H).

(S)-7-(3-(2-(1H-pyrrolo[2,3-b]pyridin-3-yl)thiazol-4-yl)phenyl)-6,7-dihydro-5H-cyclopenta[b]pyridin-7-ol. The enantiomers of (R,S)-7-(3-(2-(1H-pyrrolo[2,3-b]pyridin-3-yl)thiazol-4-yl)phenyl)-6,7-dihydro-5H-cyclopenta[b]pyridin-7-ol (85 mg, 0.21 mmol) were separated by SFC using an SFC column, such as a Daicel Chiralpak AS-H 250 mm×30 mm, 5 μm column (isocratic elution: EtOH (containing 0.1% of 25% aqueous NH₃): supercritical CO₂, 50%: 50%) to yield (S)-7-(3-(2-(1H-pyrrolo[2,3-b]pyridin-3-yl)thiazol-4-yl)phenyl)-6,7-dihydro-5H-cyclopenta[b]pyridin-7-ol as the first eluting enantiomer and (R)-7-(3-(2-(1H-pyrrolo[2,3-b]pyridin-3-yl)thiazol-4-yl)phenyl)-6,7-dihydro-5H-cyclopenta[b]pyridin-7-ol (Example 2) as the second eluting enantiomer. The pure fractions of (S)-7-(3-(2-(1H-pyrrolo[2,3-b]pyridin-3-yl)thiazol-4-yl)phenyl)-6,7-dihydro-5H-cyclopenta[b]pyridin-7-ol were combined and concentrated under reduced pressure. The product was suspended in water (10 mL) and the mixture was frozen by insertion into a 78° C. bath. The frozen mixture was then lyophilized to yield, (S)-7-(3-(2-(1H-pyrrolo[2,3-b]pyridin-3-yl)thiazol-4-yl)phenyl)-6,7-dihydro-5H-cyclopenta[b]pyridin-7-ol, SFC Rₜ=4.218 min, (36 mg, 42%) as a pale yellow solid. LCMS (ESI): mass calcd. for C₂₄H₁₈N₄OS, 410.12; m/z found, 411.1 [M+H]⁺. ¹H NMR (400 MHz, DMSO-d₆) δ 12.28 (br s, 1H), 8.52 (dd, J=8.0, 1.6 Hz, 1H), 8.42 (d, J=4.2 Hz, 1H), 8.36 (dd, J=4.8, 1.6 Hz, 1H), 8.26 (d, J=2.4 Hz, 1H), 8.00 (s, 1H), 7.92-7.87 (m, 2H), 7.80 (d, J=6.8 Hz, 1H), 7.44-7.39 (m, 1H), 7.38-7.34 (m, 1H), 7.33-7.26 (m, 2H), 5.89 (s, 1H), 3.18-3.07 (m, 1H), 3.00-2.90 (m, 1H), 2.48-2.39 (m, 2H).

### Example 2. (R)-7-(3-(2-(1H-Pyrrolo[2,3-b]pyridin-3-yl)thiazol-4-yl)phenyl)-6,7-dihydro-5H-cyclopenta[b]pyridin-7-ol

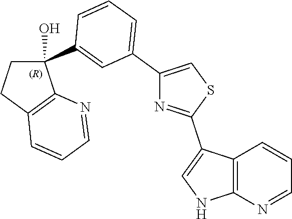

The chiral separation as described in Example 1, Step B provided (R)-7-(3-(2-(1H-pyrrolo[2,3-b]pyridin-3-yl)thiazol-4-yl)phenyl)-6,7-dihydro-5H-cyclopenta[b]pyridin-7-ol, SFC Rₜ=4.451 min (30 mg, 35%) as a white solid. LCMS (ESI): mass calcd. for C₂₄H₁₈N₄OS, 410.12; m/z found, 411.2 [M+H]⁺. ¹H NMR (400 MHz, DMSO-d₆) δ 12.28 (br s, 1H), 8.52 (d, J=8.0 Hz, 1H), 8.42 (d, J=4.4 Hz, 1H), 8.36 (d, J=4.4 Hz, 1H), 8.26 (d, J=2.4 Hz, 1H), 8.01 (s, 1H), 7.92-7.88 (m, 2H), 7.81 (d, J=7.6 Hz, 1H), 7.44-7.38 (m, 1H), 7.38-7.33 (m, 1H), 7.33-7.26 (m, 2H), 5.89 (br s, 1H), 3.17-3.07 (m, 1H), 3.00-2.90 (m, 1H), 2.49-2.39 (m, 2H).

### Example 3. (R)-3-(3-(2-(1H-Pyrrolo[2,3-b]pyridin-3-yl)thiazol-5-yl)phenyl)-3-hydroxy-1-methylpyrrolidin-2-one

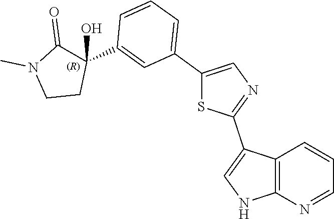

Step A. (R,S)-3-(3-(2-(1H-Pyrrolo[2,3-b]pyridin-3-yl)thiazol-5-yl)phenyl)-3-hydroxy-1-methylpyrrolidin-2-one. To a flask containing (R,S)-3-hydroxy-1-methyl-3-(3-(2-(1-(phenylsulfonyl)-1H-pyrrolo[2,3-b]pyridin-3-yl)thiazol-5-yl)phenyl)pyrrolidin-2-one (Intermediate 6, 315 mg, 0.594 mmol) and 1,4-dioxane (5 mL) was added LiOH·H₂O (1.0 mL, 3 M in H₂O, 3.0 mmol). The resultant mixture was heated at 75° C. for 1 h, then cooled to room temperature, concentrated to dryness, and triturated with water (10 mL). The suspension was isolated via filtration and the filter cake was washed with water (5 mL) and CH₃CN (5 mL) then dried under reduced pressure to yield (R,S)-3-(3-(2-(1H-pyrrolo[2,3-b]pyridin-3-yl)thiazol-5-yl)phenyl)-3-hydroxy-1-methylpyrrolidin-2-one (170 mg, 69%). LCMS (ESI): mass calcd. for C₂₁H₁₈N₄O₂S, 390.12; m/z found, 391.2 [M+H]⁺.

Step B. (R)-3-(3-(2-(1H-Pyrrolo[2,3-b]pyridin-3-yl)thiazol-5-yl)phenyl)-3-hydroxy-1-methylpyrrolidin-2-one. The enantiomers of (R,S)-3-(3-(2-(1H-Pyrrolo[2,3-b]pyridin-3-yl)thiazol-5-yl)phenyl)-3-hydroxy-1-methylpyrrolidin-2-one (160 mg, 0.41 mmol) were separated by SFC using an SFC column, such as a DAICEL CHIRALPAK AS-H 250 mm×30 mm, 5 μm column (isocratic elution:MeOH (containing 0.1% of 25% aqueous NH₃): supercritical CO₂, 50%: 50%)) to yield (R)-3-(3-(2-(1H-pyrrolo[2,3-b]pyridin-3-yl)thiazol-5-yl)phenyl)-3-hydroxy-1-methylpyrrolidin-2-one as the first eluting enantiomer and (S)-3 (3-(2-(1H-pyrrolo[2,3-b]pyridin-3-yl)thiazol-5-yl)phenyl)-3-hydroxy-1-methylpyrrolidin-2-one (Example 4) as the second eluting enantiomer. The fractions of (R)-3-(3-(2-(1H-pyrrolo[2,3-b]pyridin-3-yl)thiazol-5-yl)phenyl)-3-hydroxy-1-methylpyrrolidin-2-one were collected and concentrated under vacuum. This product was then suspended in water (10 mL) and the mixture was frozen by insertion into a −78° C. bath. The frozen mixture was then lyophilized to yield (R)-3-(3-(2-(1H-pyrrolo[2,3-b]pyridin-3-yl)thiazol-5-yl)phenyl)-3-hydroxy-1-methylpyrrolidin-2-one, SFC Rₜ=4.551 min (60.2 mg, 36%) as a yellow solid. LCMS (ESI): mass calcd. for C₂₁H₁₈N₄O₂S, 390.12; m/z found, 391.1 [M+H]⁺. ¹H NMR (400 MHz, DMSO-d₆) δ 12.35 (br. s, 1H), 8.57 (dd, J=8.0, 1.5 Hz, 1H), 8.35 (dd, J=4.8, 1.5 Hz, 1H), 8.29 (d, J=2.0 Hz, 1H), 8.21 (s, 1H), 7.68 (s, 1H), 7.65 (d, J=7.8 Hz, 1H), 7.45-7.40 (m, 1H), 7.31-7.25 (m, 2H), 6.16 (s, 1H), 3.50-3.43 (m, 1H), 3.42-3.36 (m, 1H), 2.86 (s, 3H), 2.42-2.34 (m, 1H), 2.31-2.22 (m, 1H).

### Example 4. (S)-3-(3-(2-(1H-Pyrrolo[2,3-b]pyridin-3-yl)thiazol-5-yl)phenyl)-3-hydroxy-1-methylpyrrolidin-2-one

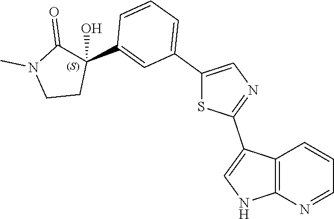

The chiral separation as described in Example 3, Step B provided (S)-3-(3-(2-(1H-pyrrolo[2,3-b]pyridin-3-yl)thiazol-5-yl)phenyl)-3-hydroxy-1-methylpyrrolidin-2-one, SFC Rₜ=5.013 min, (64.6 mg, 40%) as a yellow solid. LCMS (ESI): mass calcd. for C₂₁H₁₆N₄O₂S, 390.12; m/z found, 391.1 [M+H]⁺. ¹H NMR (400 MHz, DMSO-d₆) δ 12.35 (br s, 1H), 8.57 (dd, J=7.9, 1.4 Hz, 1H), 8.35 (dd, J=4.6, 1.4 Hz, 1H), 8.29 (d, J=2.3 Hz, 1H), 8.21 (s, 1H), 7.68 (s, 1H), 7.65 (d, J=7.8 Hz, 1H), 7.46-7.40 (m, 1H), 7.31-7.25 (m, 2H), 6.16 (s, 1H), 3.51-3.43 (m, 1H), 3.42-3.36 (m, 1H), 2.86 (s, 3H), 2.42-2.32 (m, 1H), 2.31-2.22 (m, 1H).

### Example 5. (R)-7-(3-(2-(7H-Pyrrolo[2,3-d]pyrimidin-5-yl)thiazol-4-yl)phenyl)-6,7-dihydro-5H-cyclopenta[b]pyridin-7-ol

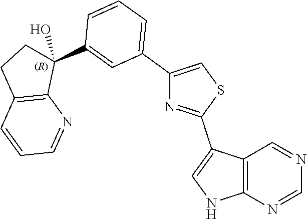

Step A. (R,S)-7-(3-(2-(7H-pyrrolo[2,3-d]pyrimidin-5-yl)thiazol-4-yl)phenyl)-6,7-dihydro-5H-cyclopenta[b]pyridin-7-ol. Aqueous NaOH (1.68 mL, 2.0 M, 3.36 mmol) was added to a solution of (R,S)-7-(3-(2-(7-tosyl-7H-pyrrolo[2,3-d]pyrimidin-5-yl)thiazol-4-yl)phenyl)-6,7-dihydro-5H-cyclopenta[b]pyridin-7-ol (Intermediate 8, 380 mg, 0.67 mmol) and 1,4-dioxane (10 mL). The resultant mixture was heated at 60° C. for 1 h. The reaction mixture was cooled to room temperature and concentrated to dryness. The resulting residue was dissolved in water (10 mL) and the pH adjusted to pH=6-7 with 1 N HCl. A suspension formed and the solids isolated by filtration. The filter cake was washed with water (20 mL), dried under vacuum to yield (R,S)-7-(3-(2-(7H-pyrrolo[2,3-d]pyrimidin-5-yl)thiazol-4-yl)phenyl)-6,7-dihydro-5H-cyclopenta[b]pyridin-7-ol (260 mg, 90%) as a brown solid. LCMS (ESI): mass calcd. for C₂₃H₁₇N₅OS, 411.12; m/z found, 412.1 [M+H]⁺.

Step B. (R)-7-(3-(2-(7H-Pyrrolo[2,3-d]pyrimidin-5-yl)thiazol-4-yl)phenyl)-6,7-dihydro-5H-cyclopenta[b]pyridin-7-ol. The enantiomers of (R,S)-7-(3-(2-(7H-pyrrolo[2,3-d]pyrimidin-5-yl)thiazol-4-yl)phenyl)-6,7-dihydro-5H-cyclopenta[b]pyridin-7-ol were separated by SFC using an SFC column, such as a YMC CHIRAL Amylose-C 250 mm×30 mm, 10 μm column (isocratic elution: EtOH (containing 0.1% of 25% aqueous NH₃): supercritical CO₂, 50%: 50%) to provide (R)-7-(3-(2-(7H-pyrrolo[2,3-d]pyrimidin-5-yl)thiazol-4-yl)phenyl)-6,7-dihydro-5H-cyclopenta[b]pyridin-7-ol as a first eluting enantiomer and (S)-7-(3-(2-(7H-pyrrolo[2,3-d]pyrimidin-5-yl)thiazol-4-yl)phenyl)-6,7-dihydro-5H-cyclopenta[b]pyridin-7-ol (Example 6) as a second eluting enantiomer. The fractions containing (R)-7-(3-(2-(7H-pyrrolo[2,3-d]pyrimidin-5-yl)thiazol-4-yl)phenyl)-6,7-dihydro-5H-cyclopenta[b]pyridin-7-ol were collected and concentrated under reduced pressure. This product was suspended in water (10 mL) and the mixture was frozen by insertion into a −78° C. bath. The frozen mixture was then lyophilized. (R)-7-(3-(2-(7H-Pyrrolo[2,3-d]pyrimidin-5-yl)thiazol-4-yl)phenyl)-6,7-dihydro-5H-cyclopenta[b]pyridin-7-ol was then further purified by SFC using an SFC column, such as a YMC CHIRAL Amylose-C 250 mm×30 mm, 10 μm column (isocratic elution: EtOH (containing 0.1% of 25% aqueous NH₃): supercritical CO₂, 50%: 50%). The fractions from this second SFC purification were collected and concentrated under reduced pressure. This product was suspended in water (10 mL) and the mixture was frozen by insertion into a −78° C. bath. The frozen mixture was then lyophilized to yield (R)-7-(3-(2-(7H-pyrrolo[2,3-d]pyrimidin-5-yl)thiazol-4-yl)phenyl)-6,7-dihydro-5H-cyclopenta[b]pyridin-7-ol, SFC Rₜ=1.501 min. (69.6 mg, 27%) as a pale yellow solid. LCMS (ESI): mass calcd. for C₂₃H₁₇N₅OS, 411.12; m/z found, 412.1 [M+H]⁺. ¹H NMR (400 MHz, DMSO-d₆) δ 12.72 (br s, 1H), 9.54 (s, 1H), 8.91 (s, 1H), 8.43 (d, J=4.4 Hz, 1H), 8.38 (d, J=2.4 Hz, 1H), 8.10-8.06 (m, 1H), 7.97 (s, 1H), 7.91 (d, J=7.6 Hz, 1H), 7.79 (d, J=7.6 Hz, 1H), 7.44-7.38 (m, 1H), 7.34-7.26 (m, 2H), 5.92 (br s, 1H), 3.16-3.07 (m, 1H), 2.99-2.89 (m, 1H), 2.57-2.51 (m, 1H), 2.47-2.39 (m, 1H).

### Example 6. (S)-7-(3-(2-(7H-Pyrrolo[2,3-d]pyrimidin-5-yl)thiazol-4-yl)phenyl)-6,7-dihydro-5H-cyclopenta[b]pyridin-7-ol

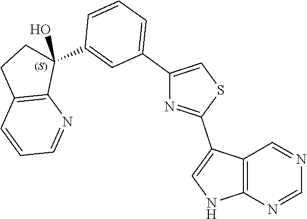

The initial chiral separation as described in Example 5, provided (S)-7-(3-(2-(7H-pyrrolo[2,3-d]pyrimidin-5-yl)thiazol-4-yl)phenyl)-6,7-dihydro-5H-cyclopenta[b]pyridin-7-ol. This product was further purified by SFC using an SFC column, such as a YMC CHIRAL Amylose-C 250 mm×30 mm, 10 μm column (isocratic elution: EtOH (containing 0.1% of 25% aqueous NH₃): supercritical CO₂, 50%: 50%). The fractions were collected and concentrated under reduced pressure. This product was suspended in water (10 mL) and the mixture was frozen by insertion into a −78° C. bath. The frozen mixture was then lyophilized to yield. Chiral SFC Rₜ=2.147 min. LCMS (ESI): mass calcd. for C₂₃H₁₇N₅OS, 411.12; m/z found, 412.1 [M+H]⁺. ¹H NMR (400 MHz, DMSO-d₆) δ 12.71 (br s, 1H), 9.52 (s, 1H), 8.90 (s, 1H), 8.41 (d, J=4.2 Hz, 1H), 8.37 (d, J=2.7 Hz, 1H), 8.09-8.04 (m, 1H), 7.95 (s, 1H), 7.89 (d, J=7.6 Hz, 1H), 7.79 (d, J=7.3 Hz, 1H), 7.43-7.36 (m, 1H), 7.33-7.25 (m, 2H), 5.91 (brs, 1H), 3.16-3.04 (m, 1H), 2.99-2.88 (m, 1H), 2.55-2.50 (m, 1H), 2.46-2.38 (m, 1H).

### Example 7. (R)-3-Hydroxy-1-methyl-3-(3-(2-(5-methyl-1H-pyrrolo[2,3-b]pyridin-3-yl)thiazol-4-yl)phenyl)pyrrolidin-2-one

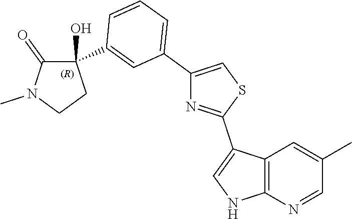

Aqueous NaOH (0.32 mL, 2 M, 0.64 mmol) was added to a solution of (R)-3-hydroxy-1-methyl-3-(3-(2-(5-methyl-1-tosyl-1H-pyrrolo[2,3-b]pyridin-3-yl)thiazol-4-yl)phenyl)pyrrolidin-2-one (Intermediate 18, 90 mg, 0.16 mmol), and 1,4-dioxane (3 mL). The mixture was heated at 70° C. for 1 h and then cooled to room temperature. The pH of the mixture was adjusted to pH=7 by the addition of 1 M HCl. This mixture was then concentrated to dryness and purified by preparative HPLC using an HPLC column, such as a Boston Prime C18 150×30 mm×5 μm column (eluent: 30% to 60%, CH₃CN and H₂O (with 0.04% NH₄OH+10 mM NH₄HCO₃)) to yield the product. The product was suspended in water (10 mL) and the mixture was frozen by insertion into a −78° C. bath. The frozen mixture was then lyophilized to yield (R)-3-hydroxy-1-methyl-3-(3-(2-(5-methyl-1H-pyrrolo[2,3-b]pyridin-3-yl)thiazol-4-yl)phenyl)pyrrolidin-2-one (22.2 mg, 34%) as a yellow solid. LCMS (ESI): mass calcd. for C₂₂H₂₀N₄O₂S, 404.13; m/z found, 405.2 [M+H]⁺. ¹H NMR (400 MHz, DMSO-d₆) δ 12.18 (br. s, 1H), 8.47 (d, J=1.3 Hz, 1H), 8.24-8.20 (m, 2H), 8.11 (s, 1H), 7.97-7.93 (m, 2H), 7.49-7.42 (m, 1H), 7.40-7.33 (m, 1H), 6.14 (s, 1H), 3.54-3.36 (m, 2H), 2.88 (s, 3H), 2.53-2.52 (m, 3H), 2.43-2.35 (m, 1H), 2.32-2.25 (m, 1H).

### Example 8. (R)-3-(3-(4-(1H-Pyrrolo[2,3-b]pyridin-3-yl)thiazol-2-yl)phenyl)-3-hydroxy-1-methylpyrrolidin-2-one

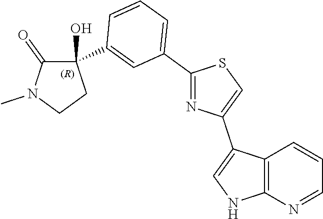

Aqueous NaOH (0.63 mL, 3 M, 1.89 mmol) was added to a solution of (R)-(3-hydroxy-1-methyl-3-(3-(4-(1-(phenylsulfonyl)-1H-pyrrolo[2,3-b]pyridin-3-yl)thiazol-2-yl)phenyl)pyrrolidin-2-one, (Intermediate 20, 200 mg, 0.38 mmol), and 1,4-dioxane (6 mL). The resultant mixture was heated at 70° C. for 1 h. The pH of the mixture was adjusted to pH=7 with the addition of 1 M HCl. This mixture was then concentrated to dryness and purified by preparative HPLC using an HPLC column, such as a YMC-Triart Prep 250×50 mm×10 mm column (eluent: 28% to 58%, CH₃CN and H₂O (with 0.04% NH₄OH+10 mM NH₄HCO₃)). The product was suspended in water (10 mL) and the mixture was frozen by insertion into a −78° C. bath. The frozen mixture was then lyophilized yield (R)-3-(3-(4-(1H-pyrrolo[2,3-b]pyridin-3-yl)thiazol-2-yl)phenyl)-3-hydroxy-1-methylpyrrolidin-2-one (50 mg, 34%) as a light yellow solid. LCMS (ESI): mass calcd. for C₂₁H₁₈N₄O₂S, 390.12; m/z found, 391.2 [M+H]⁺. ¹H NMR (400 MHz, DMSO-d₆) δ 12.12 (br. s, 1H), 8.61 (dd, J=7.8, 1.5 Hz, 1H), 8.30 (dd, J=1.5, 4.5 Hz, 1H), 8.10 (s, 2H), 8.02-7.91 (m, 2H), 7.56-7.48 (m, 1H), 7.46-7.40 (m, 1H), 7.21 (dd, J=8.0, 4.8 Hz, 1H), 6.27 (br s, 1H), 3.54-3.43 (m, 2H), 2.88 (s, 3H), 2.42-2.24 (m, 2H).

### Example 9. (R)-3-(3-(5-(1H-Pyrrolo[2,3-b]pyridin-3-yl)thiazol-2-yl)phenyl)-3-hydroxy-1-methylpyrrolidin-2-one

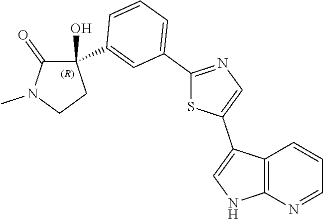

(R)-3-Hydroxy-1-methyl-3-(3-(5-(1-(phenylsulfonyl)-1H-pyrrolo[2,3-b]pyridin-3-yl)thiazol-2-yl)phenyl)pyrrolidin-2-one, (Intermediate 22, 100 mg, 0.19 mmol) was added to a mixture of aqueous NaOH (0.60 mL, 2 M, 1.2 mmol) and 1,4-dioxane (3 mL). The reaction mixture was heated at 60° C. for 1 h, then cooled to room temperature, diluted with H₂O (10 mL) and extracted with ethyl acetate:methanol (10:1, 20 mL×3). The combined organic solvent extracts were washed with H₂O (10 mL×3), concentrated to dryness, and purified by preparative HPLC using an HPLC column, such as a Boston Prime C18 150×30 mm×5 μm column (eluent: 25% to 55%, water (0.04% NH₄OH+10 mM NH₄HCO₃)-ACN). The product was suspended in water (5 mL) and the mixture was frozen by insertion into a −78° C. bath. The frozen mixture was then lyophilized to yield (R)-3-(3-(5-(1H-pyrrolo[2,3-b]pyridin-3-yl)thiazol-2-yl)phenyl)-3-hydroxy-1-methylpyrrolidin-2-one (11.3 mg, 15%) as a yellow solid. LCMS (ESI): mass calcd. for C₂₁H₁₈N₄O₂S, 390.46; m/z found, 391.2 [M+H]⁺. ¹H NMR (400 MHz, CDCl₃) δ 10.66 (br s, 1H), 8.38 (d, J=4.6 Hz, 1H), 8.04-7.89 (m, 2H), 7.87-7.79 (m, 2H), 7.49-7.36 (m, 3H), 7.26-7.21 (m, 1H), 3.68-3.48 (m, 2H), 3.07 (s, 3H), 2.70-2.56 (m, 2H).

### Example 10. (R)-7-(3-(2-(1H-Pyrrolo[2,3-b]pyridin-3-yl)thiazol-4-yl)phenyl)-6,7-dihydro-5H-pyrrolo[1,2-a]imidazol-7-ol

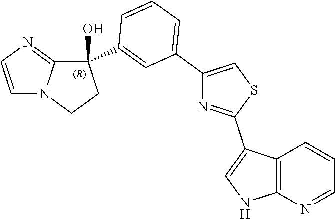

(R)-7-(3-(2-(1-(Phenylsulfonyl)-1H-pyrrolo[2,3-b]pyridin-3-yl)thiazol-4-yl)phenyl)-6,7-dihydro-5H-pyrrolo[1,2-a]imidazol-7-ol (Intermediate 23, 190 mg) was added to a mixture of aqueous NaOH (1.1 mL, 2 M, 2.2 mmol) and 1,4-dioxane (6 mL). The resultant mixture was heated at 60° C. for 2 h. The pH of the mixture was adjusted to pH=7 with 1 M HCl. The resultant solution was concentrated to dryness, and the residue that resulted was purified by preparative HPLC using an HPLC column, such as a Xtimate C18 150 mm×40 mm×10 μm column (eluent: 26% to 56%, CH₃CN and H₂O (with 0.04% NH₄OH+10 mM NH₄HCO₃)). The product was suspended in water (10 mL) and the mixture was frozen by insertion into a −78° C. bath. The frozen mixture was then lyophilized to yield (R)-7-(3-(2-(1H-pyrrolo[2,3-b]pyridin-3-yl)thiazol-4-yl)phenyl)-6,7-dihydro-5H-pyrrolo[1,2-a]imidazol-7-ol (55.4 mg, 38%) as a yellow solid. LCMS (ESI): mass calcd. for C₂₂H₁₇N₅OS, 399.12; m/z found, 400.2 [M+H]⁺. ¹H NMR (400 MHz, DMSO-d₆) δ 12.26 (s, 1H), 8.63-8.59 (m, 1H), 8.33-8.30 (m, 1H), 8.23 (s, 1H), 8.17 (s, 1H), 7.94-7.90 (m, 1H), 7.88 (s, 1H), 7.44-7.41 (m, 2H), 7.29-7.25 (m, 1H), 7.22-7.16 (m, 1H), 7.06-7.02 (m, 1H), 6.17 (s, 1H), 4.20-3.96 (m, 2H), 2.82 (t, J=6.5 Hz, 2H).

### Example 11. (R)-7-(3-(4-(5H-Pyrrolo[2,3-b]pyrazin-7-yl)thiazol-2-yl)phenyl)-6,7-dihydro-5H-pyrrolo[1,2-a]imidazol-7-ol

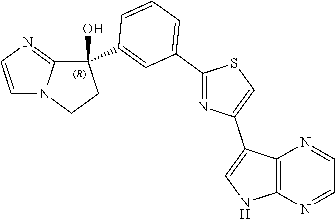

(R)-7-(3-(4-(5-Tosyl-5H-pyrrolo[2,3-b] pyrazin-7-yl)thiazol-2-yl)phenyl)-6,7-dihydro-5H-pyrrolo[1,2-a]imidazol-7-ol (Intermediate 25, 150 mg, 0.270 mmol) was added to NaOH (0.85 mL, 2 M in water, 1.7 mmol) and 1,4-dioxane (6 mL). The resultant mixture was heated at 60° C. for 2 h. The pH of mixture was adjusted to pH=7 with 1 M HCl. The mixture was concentrated to dryness and purified by preparative HPLC using an HPLC column, such as a Phenomenex Gemini-NX C18 75 mm×30 mm×3 μm column (eluent: 25% to 55%, CH₃CN and H₂O (with 0.04% NH₄OH+10 mM NH₄HCO₃)). The product was suspended in water (10 mL) and the mixture was frozen by insertion into a −78° C. bath. The frozen mixture was then lyophilized to yield (R)-7-(3-(4-(5H-pyrrolo[2,3-b]pyrazin-7-yl)thiazol-2-yl)phenyl)-6,7-dihydro-5H-pyrrolo[1,2-a]imidazol-7-ol (36.8 mg, 34%) as a yellow solid. LCMS (ESI): mass calcd. for C₂₁H₁₆N₆OS, 400.11; m/z found, 401.0 [M+H]⁺. ¹H NMR (400 MHz, DMSO-d₆) δ 12.41-12.37 (m, 1H), 8.56 (d, J=2.7 Hz, 1H), 8.40-8.35 (m, 2H), 8.25 (s, 1H), 8.22 (s, 1H), 8.00-7.96 (m, 1H), 7.52 (d, J=4.9 Hz, 2H), 7.22-7.20 (m, 1H), 7.05-7.03 (m, 1H), 6.28 (s, 1H), 4.21-4.06 (m, 2H), 2.93-2.81 (m, 2H).

### Example 12. (S)-7-(3-(4-(5H-Pyrrolo[2,3-b]pyrazin-7-yl)thiazol-2-yl)phenyl)-6,7-dihydro-5H-pyrrolo[1,2-a]imidazol-7-ol

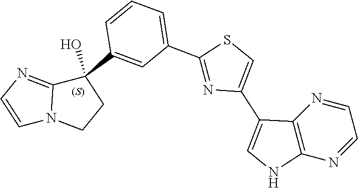

NaOH (0.49 mL, 2.0 M in water, 0.98 mmol) was added to a solution of (S)-7-(3 (4-(5-tosyl-5H-pyrrolo[2,3-b]pyrazin-7-yl)thiazol-2-yl)phenyl)-6,7-dihydro-5H-pyrrolo[1,2-a]imidazol-7-ol, (Intermediate 95, 90 mg, 0.16 mmol) and 1,4-dioxane (6 mL). The resultant mixture was heated at 60° C. for 2 h, then cooled to room temperature, concentrated to dryness, and purified by preparative HPLC using an HPLC column, such as a Xtimate C18 150×40 mm×10 μm column (eluent: 40% to 70%, CH₃CN and H₂O (with 0.04% NH₄OH+10 mM NH₄HCO₃)). The product was suspended in water (10 mL) and the mixture was frozen by insertion into a −78° C. bath. The frozen mixture was then lyophilized to yield (S)-7-(3-(4-(5H-pyrrolo[2,3-b]pyrazin-7-yl)thiazol-2-yl)phenyl)-6,7-dihydro-5H-pyrrolo[1,2-a]imidazol-7-ol (28.1 mg, 43%) as a brown solid. SFC Rₜ=1.632 min. LCMS (ESI): mass calcd. for C₂₁H₁₆N₆OS, 400.11; m/z found, 401.0 [M+H]⁺. ¹H NMR (400 MHz, DMSO-d₆) δ 12.37 (br s, 1H), 8.56 (d, J=2.4 Hz, 1H), 8.39 (s, 1H), 8.37 (d, J=2.4 Hz, 1H), 8.25 (s, 1H), 8.23-8.20 (m, 1H), 8.03-7.94 (m, 1H), 7.51 (d, J=4.9 Hz, 2H), 7.21 (d, J=1.2 Hz, 1H), 7.04 (d, J=1.0 Hz, 1H), 6.29 (s, 1H), 4.23-4.04 (m, 2H), 2.94-2.79 (m, 2H).

### Example 13. (R)-7-(3-(2-(5H-Pyrrolo[2,3-b]pyrazin-7-yl)thiazol-4-yl)phenyl)-7H-pyrrolo[1,2-a]imidazol-7-ol

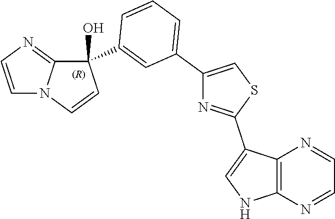

Aqueous NaOH (0.30 mL 2.0 M, 0.60 mmol) was added to a solution of (R)-7-(3-(2-(5-tosyl-5H-pyrrolo[2,3-b]pyrazin-7-yl)thiazol-4-yl)phenyl)-7H-pyrrolo[1,2-a]imidazol-7-ol, (Intermediate 29, 55 mg, 0.10 mmol) and 1,4-dioxane (6 mL). The resultant mixture was heated at 60° C. for 1 h, then cooled to room temperature, concentrated to dryness, and purified by preparative HPLC using an HPLC column, such as a Phenomenex Gemini-NX C18 75×30 mm×3 μm column (eluent: 23% to 53%, CH₃CN and H₂O (with 0.05% NH₄OH+10 mM NH₄HCO₃)). The product was suspended in water (10 mL) and the mixture was frozen by insertion into a −78° C. bath. The frozen mixture was then lyophilized to yield (R)-7-(3-(2-(5H-pyrrolo[2,3-b]pyrazin-7-yl)thiazol-4-yl)phenyl)-7H-pyrrolo[1,2-a]imidazol-7-ol (13.3 mg, 34%) as a white solid. LCMS (ESI): mass calcd. for C₂₁H₁₄N₆OS, 398.09; m/z found, 399.0 [M+H]⁺. ¹H NMR (400 MHz, DMSO-d₆) δ 12.77 (br s, 1H), 8.63-8.59 (m, 2H), 8.41 (d, J=2.5 Hz, 1H), 8.21-8.15 (m, 1H), 8.02 (s, 1H), 7.99-7.94 (m, 1H), 7.44-7.38 (m, 2H), 7.34-7.29 (m, 1H), 7.28-7.25 (m, 1H), 6.99 (s, 1H), 6.50 (s, 1H), 6.21 (d, J=4.5 Hz, 1H).

### Example 14. (S)-7-(3-(2-(5H-Pyrrolo[2,3-b]pyrazin-7-yl)thiazol-4-yl)phenyl)-7H-pyrrolo[1,2-a]imidazol-7-ol

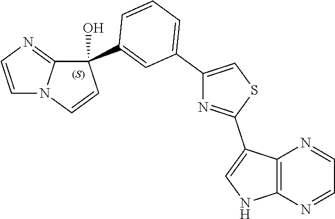

(S)-7-(3-(2-(5H-pyrrolo[2,3-b]pyrazin-7-yl)thiazol-4-yl)phenyl)-7H-pyrrolo[1,2-a]imidazol-7-ol, (15.4 mg, 39%, white solid), was prepared in a manner analogous to Example 10, except (S)-7-(3-(2-(5-tosyl-5H-pyrrolo[2,3-b]pyrazin-7-yl)thiazol-4-yl)phenyl)-7H-pyrrolo[1,2-a]imidazol-7-ol (Intermediate 31) was used in place of (R)-7-(3-(2-(5-tosyl-5H-pyrrolo[2,3-b]pyrazin-7-yl)thiazol-4-yl)phenyl)-7H-pyrrolo[1,2-a]imidazol-7-oi (Intermediate 29). LCMS(ESI): mass calcd. for C₂₁H₁₄N₆OS, 398.09; m/z found, 399.0 [M+H]⁺. ¹H NMR (400 MHz, DMSO-d₆) δ 8.63-8.59 (m, 2H), 8.42 (d, J=2.7 Hz, 1H), 8.21-8.16 (m, 1H), 8.02 (s, 1H), 7.98-7.95 (m, 1H), 7.44-7.38 (m, 2H), 7.34-7.31 (m, 1H), 7.27 (d, J=1.2 Hz, 1H), 7.01-6.99 (m, 1H), 6.53 (s, 1H), 6.25-6.19 (m, 1H).

### Example 15. (R)-7-(3-(4-(1H-Pyrrolo[2,3-b]pyridin-3-yl)thiazol-2-yl)phenyl)-6,7-dihydro-5H-pyrrolo[1,2-a]imidazol-7-ol

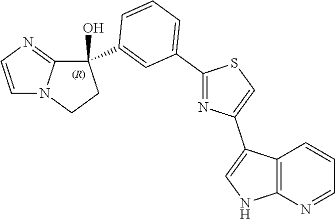

(R)-7-(3-(4-(1-(Phenylsulfonyl)-1H-pyrrolo[2,3-b]pyridin-3-yl)thiazol-2-yl)phenyl)-6,7-dihydro-5H-pyrrolo[1,2-a]imidazol-7-ol (Intermediate 32, 150 mg, 0.28 mmol) was added to a mixture of NaOH (0.87 mL, 2 M in water, 1.7 mmol) and 1,4-dioxane (6 mL). The reaction mixture was heated at 60° C. for 1.5 h, then cooled to room temperature, and the pH was adjusted to pH=7 with 1 M HCl. The resulting mixture was concentrated to dryness to a residue, and the residue was purified by preparative HPLC using an HPLC column, such as a Phenomenex Gemini-NX C18 75 mm×30 mm×3 μm column (eluent: 27% to 57%, CH₃CN and H₂O (with 0.04% NH₄OH+10 mM NH₄HCO₃)). The product was suspended in water (10 mL) and the mixture was frozen by insertion into a −78° C. bath. The frozen mixture was then lyophilized to yield (R)-7-(3-(4-(1H-pyrrolo[2,3-b]pyridin-3-yl)thiazol-2-yl)phenyl)-6,7-dihydro-5H-pyrrolo[1,2-a]imidazol-7-ol (50.5 mg, 45%) as a yellow solid. LCMS (ESI): mass calcd. for C₂₂H₁₇N₅OS, 399.12; m/z found, 400.0 [M+H]⁺. ¹H NMR (400 MHz, DMSO-d₆) δ11.97 (br s, 1H), 8.62-8.57 (m, 1H), 8.32-8.28 (m, 1H), 8.22 (s, 1H), 8.11-8.07 (m, 1H), 8.00-7.95 (m, 1H), 7.92 (s, 1H), 7.55-7.50 (m, 2H), 7.24-7.19 (m, 2H), 7.07 (s, 1H), 6.29 (s, 1H), 4.22-4.04 (m, 2H), 2.89-2.82 (m, 2H).

### Example 16. (R)-3-(3-(2-(1H-Pyrrolo[2,3-b]pyridin-3-yl)thiazol-4-yl)phenyl)-2,3-dihydrofuro[3,2-b]pyridin-3-ol

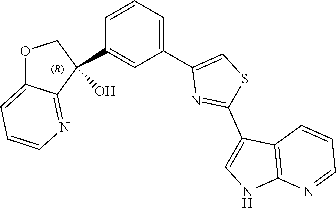

(R)-3-(3-(2-(1-(Phenylsulfonyl)-1H-pyrrolo[2,3-b]pyridin-3-yl)thiazol-4-yl)phenyl)-2,3-dihydrofuro[3,2-b]pyridin-3-ol (Intermediate 37, 120 mg, 0.22 mmol) was added to a mixture of aqueous NaOH (0.6 mL, 2 M, 1.2 mmol) and 1,4-dioxane (6 mL). The reaction mixture was heated at 60° C. for 1 h, then cooled to room temperature, and the pH was adjusted to pH=7 with the addition of 1 M HCl. The reaction mixture was concentrated to dryness under reduced pressure and purified by preparative HPLC using an HPLC column, such as a Phenomenex Gemini-NX C18 75 mm×30 mm×3 μm column (eluent: 35% to 65%, CH₃CN and H₂O (with 0.04% NH₄OH+10 mM NH₄HCO₃)). The product was suspended in water (10 mL) and the mixture was frozen by insertion into a −78° C. bath. The frozen mixture was then lyophilized to yield (R)-3-(3-(2-(1H-pyrrolo[2,3-b]pyridin-3-yl)thiazol-4-yl)phenyl)-2,3-dihydrofuro[3,2-b]pyridin-3-ol (15.1 mg, 17%) as a white solid. LCMS (ESI): mass calcd. for C₂₃H₁₈N₄O₂S, 412.10; m/z found, 413.1 [M+H]⁺. ¹H NMR (400 MHz, DMSO-d₆) δ 12.29 (br s, 1H), 8.56-8.50 (m, 1H), 8.37-8.33 (m, 1H), 8.26 (s, 1H), 8.17-8.11 (m, 2H), 7.99-7.92 (m, 2H), 7.50-7.40 (m, 3H), 7.36-7.30 (m, 1H), 7.30-7.24 (m, 1H), 6.55 (s, 1H), 4.77-4.65 (m, 2H).

### Example 17. (R)-3-(3-(2-(5H-Pyrrolo[2,3-b]pyrazin-7-yl)thiazol-4-yl)phenyl)-2,3-dihydrofuro[3,2-b]pyridin-3-ol

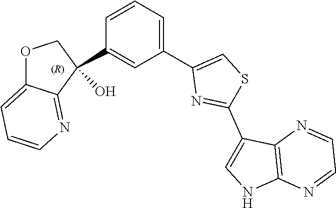

(R)-3-(3-(2-(5-Tosyl-5H-pyrrolo[2,3-b]pyrazin-7-yl)thiazol-4-yl)phenyl)-2,3-dihydrofuro[3,2-b]pyridin-3-ol (Intermediate 38, 70 mg, 0.12 mmol) was added to a mixture of aqueous NaOH solution (0.4 mL, 2 M, 0.8 mmol) and 1,4-dioxane (4 mL). The resultant mixture was heated at 60° C. for 1.5 h, then cooled to room temperature and the pH was adjusted to pH 7 with 1 M HCl. The mixture was concentrated to dryness under reduced pressure and purified by preparative HPLC using an HPLC column, such as a Boston Prime C18 150 mm×30 mm×5 μm column (eluent: 28% to 58%, CH₃CN and H₂O (with 0.04% NH₄OH+10 mM NH₄HCO₃)). The product was suspended in water (10 mL) and the mixture was frozen by insertion into a −78° C. bath. The frozen mixture was then lyophilized to yield (R)-3-(3-(2-(5H-pyrrolo[2,3-b]pyrazin-7-yl)thiazol-4-yl)phenyl)-2,3-dihydrofuro[3,2-b]pyridin-3-ol (8.7 mg, 17%) as a yellow solid. LCMS (ESI): mass calcd. for C₂₂H₁₅N₅O₂S, 413.09; m/z found, 414.0 [M+H]⁺. ¹H NMR (400 MHz, DMSO-d₆) δ 12.74 (br s, 1H), 8.63-8.60 (m, 2H), 8.42 (d, J=2.8 Hz, 1H), 8.21-8.17 (m, 1H), 8.13-8.09 (m, 1H), 8.05 (s, 1H), 7.99 (d, J=7.5 Hz, 1H), 7.50-7.40 (m, 2H), 7.40-7.35 (m, 1H), 7.32-7.27 (m, 1H), 6.51 (s, 1H), 4.74 (s, 2H).

### Example 18. (S)-7-(3-(2-(5H-Pyrrolo[2,3-b]pyrazin-7-yl-6-d)thiazol-4-yl)phenyl)-6,7-dihydro-5H-pyrrolo[1,2-a]imidazol-7-ol

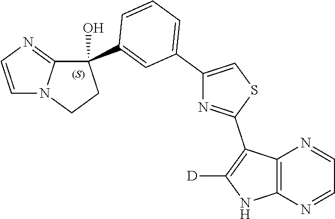

NaOH (2 M in D₂O, 840 μL, 1.68 mmol) was added to a solution of (S)-7-(3-(2-(5-tosyl-5H-pyrrolo[2,3-b]pyrazin-7-yl-6-d)thiazol-4-yl)phenyl)-6,7-dihydro-5H-pyrrolo[1,2-a]imidazol-7-ol (Intermediate 42, 230 mg, 0.41 mmol) and 1,4-dioxane (3 mL). The resultant mixture was heated at 60° C. for 2 h. The mixture was then diluted with water (10 mL) and extracted with EtOAc:MeOH (10:1, 15 mL×4). The combined organic solvent extracts were dried over anhydrous Na₂SO₄, filtered, and concentrated to yield the product, which was purified by preparative HPLC using an HPLC column, such as a Xtimate C18 150×40 mm×10 μm column (eluent: 25% to 55%, CH₃CN and H₂O (with 0.05% NH₃₊₁₀ mM NH₄HCO₃)). The product was further purified by SFC using an SFC column, such as a DAICEL CHIRALPAK AD 250 mm×30 mm, 10 μm column (isocratic elution: i-PrOH (containing 0.1% of 25% aqueous NH₃): supercritical CO₂, 50%: 50%). The pure fractions were collected and concentrated under reduced pressure. The product was suspended in water (10 mL) and the mixture was frozen by insertion into a −78° C. bath. The frozen mixture was then lyophilized to yield (S)-7-(3-(2-(5H-pyrrolo[2,3-b]pyrazin-7-yl-6-d)thiazol-4-yl)phenyl)-6,7-dihydro-5H-pyrrolo[1,2-a]imidazol-7-ol (62.7 mg, 63%) as a white solid. LCMS (ESI): mass calcd. for C₂₁H₁₅DN₆OS, 401.12; m/z found, 402.0 [M+H]⁺. ¹H NMR (400 MHz, DMSO-d₆) δ 8.62 (d, J=2.4 Hz, 1H), 8.42 (d, J=2.4 Hz, 1H), 8.24-8.13 (m, 1H), 8.02 (s, 1H), 7.99-7.93 (m, 1H), 7.50-7.34 (m, 2H), 7.20 (d, J=1.1 Hz, 1H), 7.03 (d, J=1.1 Hz, 1H), 6.18 (s, 1H), 4.25-4.02 (m, 2H), 2.97-2.78 (m, 2H).

### Example 19. (S)-7-(3-(2-(5H-Pyrrolo[2,3-b]pyrazin-7-yl-2-d)thiazol-4-yl)phenyl)-6,7-dihydro-5H-pyrrolo[1,2-a]imidazol-7-ol

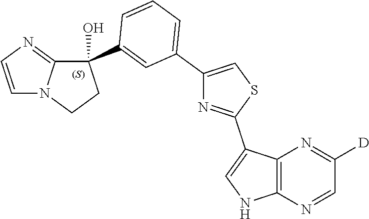

Aqueous NaOH (0.70 mL, 2.0 M, 1.4 mmol) was added to a solution of (S)-7-(3 (2-(5-tosyl-5H-pyrrolo[2,3-b]pyrazin-7-yl-2-d)thiazol-4-yl)phenyl)-6,7-dihydro-5H-pyrrolo[1,2-a]imidazol-7-ol (Intermediate 45, 130 mg, 0.234 mmol) and 1,4-dioxane (6 mL). The resultant mixture was heated at 60° C. for 1 h, then cooled to room temperature, concentrated to dryness and purified by preparative HPLC using an HPLC column Phenomenex Gemini-NX C18 75×30 mm×3 μm column (eluent: 20% to 50%, CH₃CN and H₂O with 0.04% NH₄OH+10 mM NH₄HCO₃). The product was suspended in water (10 mL) and the mixture was frozen by insertion into a −78° C. bath. The frozen mixture was then lyophilized to yield (S)-7-(3-(2-(5H-pyrrolo[2,3-b]pyrazin-7-yl-2-d)thiazol-4-yl)phenyl)-6,7-dihydro-5H-pyrrolo[1,2-a]imidazol-7-ol (42.2 mg, 45%) as a white solid. LCMS (ESI): mass calcd. for C₂₁H₁₅DN₆OS, 401.12; m/z found, 402.0 [M+H]⁺. ¹H NMR (400 MHz, DMSO-d₆) δ12.78 (s, 1H), 8.61 (s, 1H), 8.42 (s, 1H), 8.22-8.18 (m, 1H), 8.02 (s, 1H), 7.98-7.94 (m, 1H), 7.46-7.39 (m, 2H), 7.19 (d, J=1.0 Hz, 1H), 7.02 (d, J=1.0 Hz, 1H), 6.16 (s, 1H), 4.21-4.05 (m, 2H), 2.95-2.78 (m, 2H). ¹H

### Example 20. (R)-7-(3-(2-(7H-Pyrrolo[2,3-d]pyrimidin-5-yl)thiazol-4-yl)phenyl)-6,7-dihydro-5H-pyrrolo[1,2-a]imidazol-7-ol

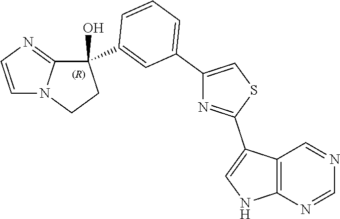

(R)-7-(3-(2-(7-Tosyl-7H-pyrrolo[2,3-d]pyrimidin-5-yl)thiazol-4-yl)phenyl)-6,7-dihydro-5H-pyrrolo[1,2-a]imidazol-7-ol (Intermediate 46, 80 mg, 0.14 mmol) was added to a mixture of NaOH (0.45 mL, 2 M in water, 0.90 mmol) and 1,4-dioxane (6 mL). The reaction mixture was heated at 60° C. for 1.5 h, then cooled to room temperature. The pH of the mixture was adjusted to pH=7 with the addition of 1 M HCl. The reaction mixture was then concentrated under reduced pressure and combined with another batch containing (50 mg). The combined products were purified by preparative HPLC using a Phenomenex Gemini-NX C18 75 mm×30 mm×3 μm column (eluent: 16% to 46%, CH₃CN and H₂O with 0.04% NH₄OH+10 mM NH₄HCO₃). The product was suspended in water (10 mL) and the mixture was frozen by insertion into a −78° C. bath. The frozen mixture was then lyophilized to yield (R)-7-(3-(2-(7H-pyrrolo[2,3-d]pyrimidin-5-yl)thiazol-4-yl)phenyl)-6,7-dihydro-5H-pyrrolo[1,2-a]imidazol-7-ol (27.0 mg) as a yellow solid. LCMS (ESI): mass calcd. for C₂₁H₁₆N₆OS, 400.11; m/z found, 401.0 [M+H]⁺. ¹H NMR (400 MHz, DMSO-d₆) δ 12.73 (br s, 1H), 9.66 (s, 1H), 8.92 (s, 1H), 8.40 (s, 1H), 8.31-8.27 (m, 1H), 8.01 (s, 1H), 8.00-7.95 (m, 1H), 7.48-7.45 (m, 2H), 7.22 (d, J=1.0 Hz, 1H), 7.13-7.10 (m, 1H), 6.23 (s, 1H), 4.22-4.02 (m, 2H), 2.87 (t, J=6.4 Hz, 2H).

### Example 21. (S)-7-(3-(2-(7H-Pyrrolo[2,3-d]pyrimidin-5-yl)thiazol-4-yl)phenyl)-6,7-dihydro-5H-pyrrolo[1,2-a]imidazol-7-ol

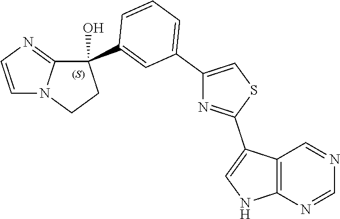

Aqueous NaOH (0.60 mL, 2.0 M, 1.2 mmol) was added to a solution of (S)-7-(3-(2-(7-tosyl-7H-pyrrolo[2,3-d]pyrimidin-5-yl)thiazol-4-yl)phenyl)-6,7-dihydro-5H-pyrrolo[1,2-a]imidazol-7-ol (Intermediate 47, 110 mg, 0.20 mmol) and 1,4-dioxane (6 mL). The resultant mixture was heated at 60° C. for 2 h, cooled to room temperature and concentrated to dryness. The product was purified by preparative HPLC using a Phenomenex Gemini NX-C18 75×30 mm×3 μm column (eluent: 20% to 50%, CH₃CN and H₂O with 0.04% NH₄OH+10 mM NH₄HCO₃). The product was suspended in water (10 mL) and the mixture was frozen by insertion into a −78° C. bath. The frozen mixture was then lyophilized to yield (S)-7-(3-(2-(7H-pyrrolo[2,3-d]pyrimidin-5-yl)thiazol-4-yl)phenyl)-6,7-dihydro-5H-pyrrolo[1,2-a]imidazol-7-ol (20 mg, 25%) as a brown solid. LCMS (ESI): mass calcd. for C₂₁H₁₆N₆OS, 400.11; m/z found, 401.0 [M+H]⁺. ¹H NMR (400 MHz, DMSO-d₆) δ 12.73 (br s, 1H), 9.65 (s, 1H), 8.91 (s, 1H), 8.39 (s, 1H), 8.31-8.23 (m, 1H), 8.00 (s, 1H), 7.98-7.94 (m, 1H), 7.49-7.43 (m, 2H), 7.23-7.20 (m, 1H), 7.12-7.09 (m, 1H), 6.23 (s, 1H), 4.21-4.13 (m, 1H), 4.09-4.02 (m, 1H), 2.86 (t, J=6.5 Hz, 2H)

### Example 22. (R)-7-(3-(4-(1H-Pyrrolo[2,3-b]pyridin-3-yl)thiazol-2-yl)phenyl)-6,7-dihydro-5H-cyclopenta[b]pyridin-7-ol

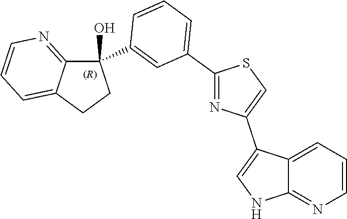

Step A. (R,S)-7-(3-(4-(1H-pyrrolo[2,3-b]pyridin-3-yl)thiazol-2-yl)phenyl)-6,7-dihydro-5H-cyclopenta[b]pyridin-7-ol. To a microwave tube containing (R,S)-7-(3-(4-(1-(phenylsulfonyl)-1H-pyrrolo[2,3-b]pyridin-3-yl)thiazol-2-yl)phenyl)-6,7-dihydro-5H-cyclopenta[b]pyridin-7-ol, (Intermediate 49, 700 mg, 1.27 mmol) and 1,4-dioxane (10 mL) that had been cooled to 0° C. was added aqueous NaOH (3.2 mL, 2M, 6.4 mmol). The resultant mixture was then heated at 70° C. for 2 h, cooled to room temperature and concentrated to dryness. The product was purified by preparative HPLC using a Boston Prime C18 150×30 mm×5 μm column (eluent: 40% to 70%, CH₃CN and H₂O (with 0.04% NH₄OH+10 mM NH₄HCO₃)). The product was suspended in water (10 mL) and the mixture was frozen by insertion into a −78° C. bath. The frozen mixture was then lyophilized to yield (R,S)-7-(3-(4-(1H-pyrrolo[2,3-b]pyridin-3-yl)thiazol-2-yl)phenyl)-6,7-dihydro-5H-cyclopenta[b]pyridin-7-ol (200 mg, 36%) as a white solid. LCMS (ESI): mass calcd. for C₂₄H₁₈N₄OS, 410.12; m/z found, 411.2 [M+H]⁺.

Step B. (R)-7-(3-(4-(1H-Pyrrolo[2,3-b]pyridin-3-yl)thiazol-2-yl)phenyl)-6,7-dihydro-5H-cyclopenta[b]pyridin-7-ol. The enantiomers of (R,S)-7-(3-(4-(1H-pyrrolo[2,3-b]pyridin-3-yl)thiazol-2-yl)phenyl)-6,7-dihydro-5H-cyclopenta[b]pyridin-7-ol (200 mg, 0.49 mmol) were separated by SFC using an SFC column, such as a DAICELCHIRALPAK AD 250 mm×30 mm, 10 μm column (isocratic elution: i-PrOH (containing 0.1% of 25% aqueous NH₃): supercritical CO₂, 60%: 40%) to provide the first eluting enantiomer (R)-7-(3-(4 (1H-pyrrolo[2,3-b]pyridin-3-yl)thiazol-2-yl)phenyl)-6,7-dihydro-5H-cyclopenta[b]pyridin-7-ol and the second eluting enantiomer (S)-7-(3-(4-(1H-pyrrolo[2,3-b]pyridin-3-yl)thiazol-2-yl)phenyl)-6,7-dihydro-5H-cyclopenta[b]pyridin-7-ol (Example 23). The fractions containing (R)-7-(3-(4-(1H-pyrrolo[2,3-b]pyridin-3-yl)thiazol-2-yl)phenyl)-6,7-dihydro-5H-cyclopenta[b]pyridin-7-ol were collected and concentrated under reduced pressure. This product was suspended in water (20 mL) and the mixture was frozen by insertion into a 78° C. bath. The frozen mixture was then lyophilized to yield (R)-7-(3-(4-(1H-pyrrolo[2,3-b]pyridin-3-yl)thiazol-2-yl)phenyl)-6,7-dihydro-5H-cyclopenta[b]pyridin-7-ol, (63.9 mg, 32%) as a white solid. SFC Rₜ=2.498 min, LCMS (ESI): mass calcd. for C₂₄H₁₈N₄OS, 410.12; m/z found, 411.0 [M+H]⁺. ¹H NMR (400 MHz, DMSO-d₆) δ 11.97 (s, 1H), 8.54 (dd, J=7.9, 1.3 Hz, 1H), 8.42-8.36 (m, 1H), 8.34-8.28 (m, 1H), 8.14-8.05 (m, 2H), 7.96-7.86 (m, 2H), 7.79 (s, 1H), 7.46 (s, 1H), 7.39-7.34 (m, 1H), 7.32-7.27 (m, 1H), 7.23-7.18 (m, 1H), 6.01 (s, 1H), 3.16-3.10 (m, 1H), 3.00-2.93 (m, 1H), 2.47-2.38 (m, 2H).

### Example 23. (S)-7-(3-(4-(1H-Pyrrolo[2,3-b]pyridin-3-yl)thiazol-2-yl)phenyl)-6,7-dihydro-5H-cyclopenta[b]pyridin-7-ol

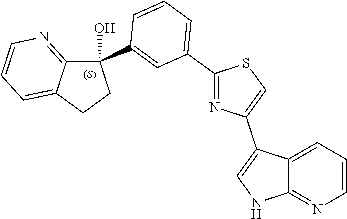

The chiral SFC separation described in Example 22, Step B yielded (S)-7-(3-(4-(1H-pyrrolo[2,3-b]pyridin-3-yl)thiazol-2-yl)phenyl)-6,7-dihydro-5H-cyclopenta[b]pyridin-7-ol (85.9 mg, 43%) as a white solid. SFC Rₜ=2.983 min. LCMS (ESI): mass calcd. for C₂₄H₁₈N₄OS, 410.12; m/z found, 411.0 [M+H]⁺. ¹H NMR (400 MHz, DMSO-d₆) δ 11.94 (br s, 1H), 8.54 (dd, J=7.8, 1.2 Hz, 1H), 8.46-8.38 (m, 1H), 8.34-8.26 (m, 1H), 8.15-8.02 (m, 2H), 7.90 (s, 2H), 7.80 (d, J=7.6 Hz, 1H), 7.49-7.43 (m, 1H), 7.37 (s, 1H), 7.32-7.26 (m, 1H), 7.23-7.17 (m, 1H), 5.99 (s, 1H), 3.14-3.07 (m, 1H), 3.00-2.93 (m, 1H), 2.47-2.40 (m, 2H).

### Example 24. (R)-7-(3-(2-(5-Fluoro-1H-pyrrolo[2,3-b]pyridin-3-yl)thiazol-4-yl)phenyl)-6,7-dihydro-5H-pyrrolo[1,2-a]imidazol-7-ol

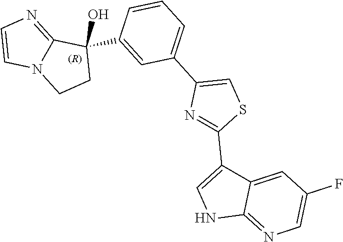

Aqueous NaOH (0.5 mL, 2 M, 1.0 mmol) was added to (R)-7-(3-(2-(5-fluoro-1-tosyl-1H-pyrrolo[2,3-b]pyridin-3-yl)thiazol-4-yl)phenyl)-6,7-dihydro-5H-pyrrolo[1,2-a]imidazol-7-ol (Intermediate 51, 100 mg, 0.18 mmol) and 1,4-dioxane (2 mL). The resultant mixture was heated at 70° C. for 1 h, cooled to room temperature and concentrated to dryness. The product was purified by preparative HPLC using a Phenomenex Gemini-NX C18 75 mm×30 mm×3 μm column (eluent: 30% to 60%, CH₃CN and H₂O (with 0.04% NH₄OH+10 mM NH₄HCO₃). The product was suspended in water (10 mL) and the mixture was frozen by insertion into a −78° C. bath. The frozen mixture was then lyophilized to yield (R)-7-(3-(2-(5-fluoro-1H-pyrrolo[2,3-b]pyridin-3-yl)thiazol-4-yl)phenyl)-6,7-dihydro-5H-pyrrolo[1,2-a]imidazol-7-ol (19.4 mg, 27%) as a brown solid. LCMS (ESI): mass calcd. for C₂₂H₁₈FN₅OS, 417.11; m/z found, 418.0 [M+H]⁺. ¹H NMR (400 MHz, DMSO-d₆) δ 12.40 (br s, 1H), 8.45-8.34 (m, 3H), 8.25 (s, 1H), 7.99-7.90 (m, 2H), 7.49-7.42 (m, 2H), 7.19 (s, 1H), 7.07 (s, 1H), 6.19 (br. s, 1H), 4.22-4.12 (m, 1H), 4.11-4.01 (m, 1H), 2.86 (t, J=6.5 Hz, 2H).

### Example 25. (R)-7-(3-(2-(5-(Trifluoromethyl)-1H-pyrrolo[2,3-b]pyridin-3-yl)thiazol-4-yl)phenyl)-6,7-dihydro-5H-pyrrolo[1,2-a]imidazol-7-ol

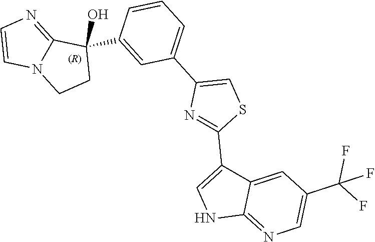

(R)-7-(3-(2-(1-Tosyl-5-(trifluoromethyl)-1H-pyrrolo[2,3-b]pyridin-3-yl)thiazol-4-yl)phenyl)-6,7-dihydro-5H-pyrrolo[1,2-a]imidazol-7-ol (Intermediate 53, 140 mg, 0.23 mmol) in 1,4-dioxane (3 mL) was cooled to 0° C.; and aqueous NaOH (0.56 mL, 2 M, 1.12 mmol) was added. The suspension was then heated at 70° C. for 2 h. The suspension was cooled to room temperature and concentrated to dryness. The product was purified by preparative HPLC using a Phenomenex Gemini-NX C18 75 mm×30 mm×3 μm column (eluent: 42% to 52%, CH₃CN and H₂O (with 0.04% NH₄OH+10 mM NH₄HCO₃)). The product was combined with another batch of material and suspended in water (10 mL) and the mixture was frozen by insertion into a −78° C. bath. The frozen mixture was then lyophilized to yield (R)-7-(3-(2-(5-(trifluoromethyl)-1H-pyrrolo[2,3-b]pyridin-3-yl)thiazol-4-yl)phenyl)-6,7-dihydro-5H-pyrrolo[1,2-a]imidazol-7-ol, (43.8 mg) as a white solid. LCMS (ESI): mass calcd. for C₂₃H₁₆F₃N₅OS, 467.10; m/z found, 468.0 [M+H]⁺. ¹H NMR (400 MHz, DMSO-d₆) δ 12.83 (br s, 1H), 8.96-8.91 (m, 1H), 8.75-8.70 (m, 1H), 8.53 (s, 1H), 8.20 (s, 1H), 7.99 (s, 1H), 7.96-7.90 (m, 1H), 7.51-7.44 (m, 2H), 7.16 (5, 1H), 7.00 (s, 1H), 6.17 (s, 1H), 4.21-4.15 (m, 1H), 4.11-4.04 (m, 1H), 2.96-2.84 (m, 2H).

### Example 26. (R)-3-(4-(3-(7-Hydroxy-6,7-dihydro-5H-pyrrolo[1,2-a]imidazol-7-yl)phenyl)thiazol-2-yl)-1H-pyrrolo[2,3-b]pyridine-5-carbonitrile

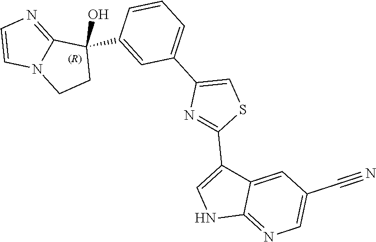

(R)-3-(4-(3-(7-Hydroxy-6,7-dihydro-5H-pyrrolo[1,2-a]imidazol-7-yl)phenyl)thiazol-2-yl)-1-tosyl-1H-pyrrolo[2,3-b]pyridine-5-carbonitrile (Intermediate 55, 110 mg, 0.19 mmol) was added to a mixture of NaOH (0.57 mL, 2 M in water, 1.1 mmol) and 1,4-dioxane (8 mL). The reaction mixture was heated at 60° C. for 1 h. The reaction mixture was cooled to room temperature, concentrated to dryness under reduced pressure. The product was purified by preparative HPLC using a Phenomenex Gemini-NX C18 75 mm×30 mm×3 μm column (eluent: 30% to 60%, CH₃CN: water (0.04% NH₄OH+10 mM NH₄HCO₃)). The product was suspended in water (5 mL) and the mixture was frozen by insertion into a −78° C. bath. The frozen mixture was then lyophilized to yield (R)-3-(4-(3-(7-hydroxy-6,7-dihydro-5H-pyrrolo[1,2-a]imidazol-7-yl)phenyl)thiazol-2-yl)-1H-pyrrolo[2,3-b]pyridine-5-carbonitrile (21.8 mg, 27%) as a white solid. LCMS (ESI): mass calcd. for C₂₃H₁₆N₆OS, 424.11; m/z found, 425.0 [M+H]⁺. ¹H NMR (400 MHz, DMSO-d₆) δ 12.89-12.11 (m, 1H), 8.98 (d, J=2.0 Hz, 1H), 8.76 (d, J=2.0 Hz, 1H), 8.53 (s, 1H), 8.18 (s, 1H), 8.03-7.94 (m, 2H), 7.52-7.43 (m, 2H), 7.20 (s, 1H), 7.10 (s, 1H), 6.21 (s, 1H), 4.22-4.14 (m, 1H), 4.11-4.05 (m, 1H), 2.93-2.85 (m, 2H).

### Example 27. (R)-7-(3-(2-(5-Chloro-1H-pyrrolo[2,3-b]pyridin-3-yl)thiazol-4-yl)phenyl)-6,7-dihydro-5H-pyrrolo[1,2-a]imidazol-7-ol

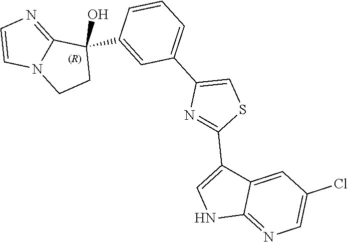

Aqueous NaOH (0.43 mL, 2 M, 0.86 mmol) was added to a mixture of (R)-7-(3-(2-(5-chloro-1-tosyl-1H-pyrrolo[2,3-b]pyridin-3-yl)thiazol-4-yl)phenyl)-6,7-dihydro-5H-pyrrolo[1,2-a]imidazol-7-ol (Intermediate 57, 100 mg, 0.17 mmol) and 1,4-dioxane (1 mL). The resultant mixture was heated at 70° C. for 2 h, then cooled to room temperature and concentrated to dryness under reduced pressure. The material was purified by preparative HPLC using a Phenomenex Gemini-NX C18 75 mm×30 mm×3 μm column (eluent: 33% to 63%, CH₃CN and H₂O with (0.04% NH₄OH+10 mM NH₄HCO₃)). The product was suspended in water (10 mL) and the mixture was frozen by inserting it into a −78° C. bath, and then concentrated to dryness to yield (R)-7-(3-(2-(5-chloro-1H pyrrolo[2,3-b]pyridin-3-yl)thiazol-4-yl)phenyl)-6,7-dihydro-5H-pyrrolo[1,2-a]imidazol-7-ol (15 mg, 20%) as a white solid. LCMS (ESI): mass calcd. for C₂₂H₁₆ClN₅OS, 433.08; m/z found, 434.0 [M+H]⁺. ¹H NMR (400 MHz, DMSO-d₆) δ 12.57 (br s, 1H), 8.63 (d, J=2.3 Hz, 1H), 8.40 (d, J=2.8 Hz, 1H), 8.37 (d, J=2.3 Hz, 1H), 8.20-8.17 (m, 1H), 7.96-7.92 (m, 2H), 7.49-7.46 (m, 2H), 7.19 (s, 1H), 7.05 (s, 1H), 6.20 (s, 1H), 4.22-4.15 (m, 1H), 4.12-4.05 (m, 1H), 2.90-2.84 (m, 2H).

### Example 28. (R)-7-(3-(2-(5-(Methylsulfonyl)-1H-pyrrolo[2,3-b]pyridin-3-yl)thiazol-4-yl)phenyl)-6,7-dihydro-5H-pyrrolo[1,2-a]imidazol-7-ol

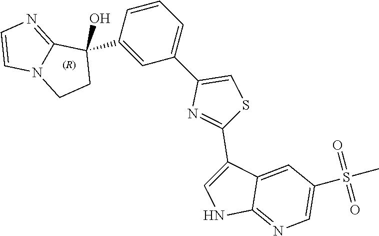

(R)-7-(3-(2-(5-(Methylsulfonyl)-1-tosyl-1H-pyrrolo[2,3-b]pyridin-3-yl)thiazol-4-yl)phenyl)-6,7-dihydro-5H-pyrrolo[1,2-a]imidazol-7-ol (Intermediate 59, 100 mg, 0.16 mmol) was added to a mixture of aqueous NaOH (0.4 mL, 2 M, 0.8 mmol) and 1,4-dioxane (4 mL). The resulting mixture was heated at 60° C. for 1.5 h, and then cooled to room temperature. The pH of the reaction mixture was adjusted to pH=7 with 2 M aqueous HCl. This mixture was concentrated to dryness under reduced pressure and purified by preparative HPLC using a Phenomenex Gemini-NX C18 75 mm×30 mm×3 μm (eluent: 22% to 52%, CH₃CN and H₂O (with 0.05% NH₄OH+10 mM NH₄HCO₃)). The product was suspended in water (10 mL) and the mixture was frozen by insertion into a −78° C. bath. The frozen mixture was then lyophilized to yield (R)-7-(3-(2-(5-(methylsulfonyl)-1H-pyrrolo[2,3-b]pyridin-3-yl)thiazol-4-yl)phenyl)-6,7-dihydro-5H-pyrrolo[1,2-a]imidazol-7-ol (12.4 mg, 16%) as a white solid. LCMS (ESI): mass calcd. for C₂₃H₁₉N₅O₃S₂, 477.09; m/z found, 478.0 [M+H]⁺. ¹H NMR (400 MHz, DMSO-d₆) δ 12.95 (br s, 1H), 9.14-9.10 (m, 1H), 8.91-8.81 (m, 1H), 8.55 (s, 1H), 8.15 (s, 1H), 8.01 (s, 1H), 7.99-7.94 (m, 1H), 7.57-7.53 (m, 1H), 7.51-7.45 (m, 1H), 7.18 (s, 1H), 7.00 (s, 1H), 6.18 (s, 1H), 4.24-4.10 (m, 2H), 3.35 (s, 3H), 3.00-2.84 (m, 2H).

### Example 29. (R)-7-(3-(2-(4-Fluoro-1H-pyrrolo[2,3-b]pyridin-3-yl)thiazol-4-yl)phenyl)-6,7-dihydro-5H-pyrrolo[1,2-a]imidazol-7-ol

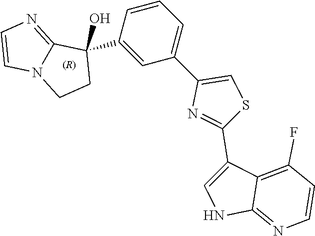

Sodium hydroxide (0.53 mL, 2 M in water, 1.1 mmol) was added to a solution of (R)-7-(3-(2-(4-fluoro-1-tosyl-1H-pyrrolo[2,3-b]pyridin-3-yl)thiazol-4-yl)phenyl)-6,7-dihydro-5H-pyrrolo[1,2-a]imidazol-7-ol (Intermediate 61, 100 mg, 0.18 mmol) and 1,4-dioxane (6 mL). The resultant mixture was heated at 60° C. for 1 h and then cooled to room temperature. The mixture was concentrated to dryness under reduced pressure and purified by preparative HPLC using an HPLC column, such as a Phenomenex Gemini NX-C18 75 mm×30 mm×3 μm (eluent: 30% to 60%, CH₃CN and H₂O (with 0.04% NH₄OH+10 mM NH₄HCO₃)). The product was suspended in water (6 mL) and the mixture was frozen by insertion into a −78° C. bath. The frozen mixture was then lyophilized to yield (R)-7-(3-(2-(4-fluoro-1H-pyrrolo[2,3-b]pyridin-3-yl)thiazol-4-yl)phenyl)-6,7-dihydro-5H-pyrrolo[1,2-a]imidazol-7-ol (27.4 mg, 38%) as a white solid. LCMS (ESI): mass calcd. for C₂₂H₁₆FN₅OS, 417.11; m/z found, 418.0 [M+H]⁺. ¹H NMR (400 MHz, DMSO-d₆) δ 12.68 (s, 1H), 8.33 (dd, J=7.8, 5.4 Hz, 1H), 8.24 (s, 1H), 8.15 (s, 1H), 7.97 (s, 1H), 7.96-7.91 (m, 1H), 7.49-7.38 (m, 2H), 7.19 (d, J=1.0 Hz, 1H), 7.13 (dd, J=11.0, 5.4 Hz, 1H), 7.01 (d, J=1.0 Hz, 1H), 6.18 (s, 1H), 4.20-4.12 (m, 1H), 4.12-4.04 (m, 1H), 2.90-2.81 (m, 2H).

### Example 30. (R)-7-(3-(5-(1H-Pyrrolo[2,3-b]pyridin-3-yl)furan-3-yl)phenyl)-6,7-dihydro-5H-pyrrolo[1,2-a]imidazol-7-ol

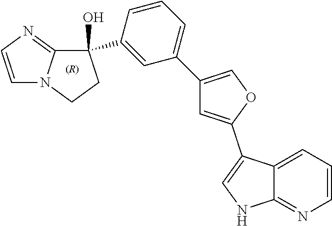

Aqueous NaOH (0.4 mL, 2 M, 0.8 mmol) was added to a solution of (R)-7-(3-(5-(1-tosyl-1H-pyrrolo[2,3-b]pyridin-3-yl)furan-3-yl)phenyl)-6,7-dihydro-5H-pyrrolo[1,2-a]imidazol-7-ol (Intermediate 62, 70 mg, 0.13 mmol) and 1,4-dioxane (8 mL). The resulting mixture was heated at 70° C. for 0.7 h and then cooled to room temperature. The solvent was removed under reduced pressure and the product was purified by preparative HPLC using a Boston Prime C18 150 mm×30 mm×5 μm column (eluent: 35% to 65%, CH₃CN and H₂O (with 0.05% NH₄OH+10 mM NH₄HCO₃)). The product was suspended in water (5 mL) and the mixture was frozen by insertion into a −78° C. bath. The frozen mixture was then lyophilized to yield (R)-7-(3-(5-(1H-pyrrolo[2,3-b]pyridin-3-yl)furan-3-yl)phenyl)-6,7-dihydro-5H-pyrrolo[1,2-a]imidazol-7-ol (12.4 mg, 24%) as a white solid. LCMS (ESI): mass calcd. for C₂₃H₁₈N₄O₂, 382.14; m/z found, 383.0 [M+H]⁺. ¹H NMR (400 MHz, DMSO-d₆) δ 12.01 (s, 1H), 8.42 (d, J=7.1 Hz, 1H), 8.34-8.28 (m, 1H), 8.13 (s, 1H), 7.90 (d, J=2.4 Hz, 1H), 7.81 (s, 1H), 7.66-7.57 (m, 1H), 7.41-7.31 (m, 2H), 7.25-7.15 (m, 3H), 7.01 (s, 1H), 6.12 (s, 1H), 4.21-4.12 (m, 1H), 4.10-4.01 (m, 1H), 2.96-2.86 (m, 1H), 2.85-2.78 (m, 1H).

### Example 31. (S)-4-(3-(2-(1H-Pyrrolo[2,3-b]pyridin-3-yl)thiazol-4-yl)phenyl)-5,6-dihydro-4H-cyclopenta[d]thiazol-4-ol

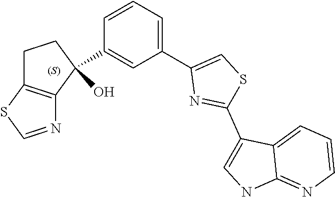

Step A. (R,S)-4-(3-(2-(1H-Pyrrolo[2,3-b]pyridin-3-yl)thiazol-4-yl)phenyl)-5,6-dihydro-4H-cyclopenta[d]thiazol-4-ol. Sodium hydroxide (5.0 mL, 10 mmol, 2.0 M in H₂O) was added to a mixture of (R,S)-4-(3-(2-(1-(phenylsulfonyl)-1H-pyrrolo[2,3-b]pyridin-3-yl)thiazol-4-yl)phenyl)-5,6-dihydro-4H-cyclopenta[d]thiazol-4-ol (Intermediate 64, 1.19 g, 2.14 mmol) and 1,4-dioxane (30 mL). The resultant mixture was heated at 60° C. for 2 h, and then cooled to room temperature and concentrated under reduced pressure to yield (R,S)-4-(3-(2-(1H-pyrrolo[2,3-b]pyridin-3-yl)thiazol-4-yl)phenyl)-5,6-dihydro-4H-cyclopenta[d]thiazol-4-ol (2.0 g) as a black oil. LCMS (ESI): mass calcd. for C₂₂H₁₆N₄OS₂, 416.08; m/z found, 417.0 [M+1]⁺.

Step B. (S)-4-(3-(2-(1H-Pyrrolo[2,3-b]pyridin-3-yl)thiazol-4-yl)phenyl)-5,6-dihydro-4H-cyclopenta[d]thiazol-4-oi. The enantiomers of (R,S)-4-(3-(2-(1H-pyrrolo[2,3-b]pyridin-3-yl)thiazol-4-yl)phenyl)-5,6-dihydro-4H-cyclopenta[d]thiazol-4-ol (2.0 g, 4.8 mmol) were separated by SFC using an SFC column, such as a DAICEL CHIRALPAK AS 250 mm×30 mm×10 μm column (isocratic elution: EtOH (containing 0.1% of 25% aqueous NH₃): supercritical CO₂, 50%: 50%) to provide the first eluting enantiomer, (S)-4-(3-(2-(1H-pyrrolo[2,3-b]pyridin-3-yl)thiazol-4-yl)phenyl)-5,6-dihydro-4H-cyclopenta[d]thiazol-4-ol and the second eluting enantiomer (R)-4-(3-(2-(1H-pyrrolo[2,3-b]pyridin-3-yl)thiazol-4-yl)phenyl)-5,6-dihydro-4H-cyclopenta[d]thiazol-4-ol (Example 32) The (S)-4-(3-(2-(1H-Pyrrolo[2,3-b]pyridin-3-yl)thiazol-4-yl)phenyl)-5,6-dihydro-4H-cyclopenta[d]thiazol-4-ol enantiomer was further purified by preparative HPLC over an Xtimate C18 150×40 mm×10 μm column (eluent: 35% to 65%, CH₃CN and H₂O (with 0.04% NH₃·H₂O+10 mM NH₄HCO₃). The pure fractions were collected and concentrated under reduced pressure. The product was suspended in water (10 mL) and the mixture was frozen by inserting it into a −78° C. bath, and then lyophilized to yield (S)-4-(3-(2-(1H-pyrrolo[2,3-b]pyridin-3-yl)thiazol-4-yl)phenyl)-5,6-dihydro-4H-cyclopenta[d]thiazol-4-ol (100 mg, 11%) as a white solid. SFC Rₜ=4.410 min, LCMS (ESI): mass calcd. for C₂₂H₁₆N₄OS₂, 416.08; m/z found, 417.0 [M+H]⁺. ¹H NMR (400 MHz, DMSO-d₆) δ 12.29 (br s, 1H), 9.03 (s, 1H), 8.56 (dd, J=1.4, 7.9 Hz, 1H), 8.36 (dd, J=4.6, 1.5 Hz, 1H), 8.26 (s, 1H), 8.05 (s, 1H), 7.93-7.87 (m, 2H), 7.46-7.40 (m, 1H), 7.39-7.35 (m, 1H), 7.34-7.28 (m, 1H), 5.96 (s, 1H), 3.17-3.07 (m, 1H), 3.03-2.93 (m, 1H), 2.89-2.78 (m, 2H).

### Example 32. (R)-4-(3-(2-(1H-Pyrrolo[2,3-b]pyridin-3-yl)thiazol-4-yl)phenyl)-5,6-dihydro-4H-cyclopenta[d]thiazol-4-ol

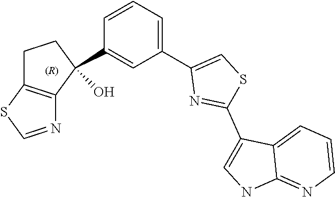

The chiral separation described in Example 31, Step B yielded (R)-4-(3-(2-(1H-pyrrolo[2,3-b]pyridin-3-yl)thiazol-4-yl)phenyl)-5,6-dihydro-4H-cyclopenta[d]thiazol-4-ol. This compound was further purified by preparative HPLC over an Xtimate C18 150×40 mm×10 μm column (eluent: 35% to 65%, CH₃CN and H₂O (with 0.04% NH₃·H₂O+10 mM NH₄HCO₃)) to provide (R)-4-(3-(2-(1H-pyrrolo[2,3-b]pyridin-3-yl)thiazol-4-yl)phenyl)-5,6-dihydro-4H-cyclopenta[d]thiazol-4-ol (120 mg, 47%) as a white solid. SFC Rₜ=5.274 min. LCMS (ESI): mass calcd. for C₂₂H₁₆N₄OS₂, 416.08; m/z found, 417.0 [M+H]⁺. ¹H NMR (400 MHz, DMSO-d₆) 512.25 (br s, 1H), 9.04 (s, 1H), 8.57 (dd, J=8.0, 1.4 Hz, 1H), 8.37 (dd, J=4.6, 1.5 Hz, 1H), 8.27 (s, 1H), 8.05 (s, 1H), 7.95-7.86 (m, 2H), 7.46-7.40 (m, 1H), 7.40-7.35 (m, 1H), 7.34-7.29 (m, 1H), 5.97 (s, 1H), 3.18-3.07 (m, 1H), 3.03-2.93 (m, 1H), 2.90-2.76 (m, 2H).

### Example 33. (R)-7-(3-(2-(1H-Pyrrolo[2,3-b]pyridin-3-yl)thiazol-4-yl)phenyl)-7H-cyclopenta[b]pyridin-7-ol

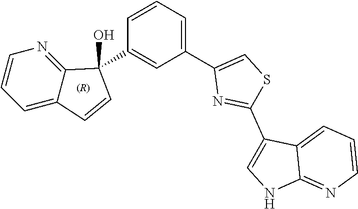

Step A. (R,S)-7-(3-(2-(1H-pyrrolo[2,3-b]pyridin-3-yl)thiazol-4-yl)phenyl)-7H-cyclopenta[b]pyridin-7-ol. Sodium hydroxide (11.6 mL, 2.0 M in H₂O, 23.3 mmol) was added to a mixture of (R,S)-7-(3-(2-(1-(phenylsulfonyl)-1H-pyrrolo[2,3-b]pyridin-3-yl)thiazol-4-yl)phenyl)-7H-cyclopenta[b]pyridin-7-ol, (Intermediate 66, 2.13 g, 3.88 mmol) and 1,4-dioxane (40 mL). The mixture was heated at 60° C. for 1 h, then cooled to room temperature and concentrated to dryness. The material was initially purified by preparative HPLC using an HPLC column, such as a YMC Triart C18 250×50 mm×7 μm column (eluent: 30% to 60%, CH₃CN and H₂O (with 0.04% NH₄OH+10 mM NH₄HCO₃)) and then further purified by preparative HPLC using an HPLC column, such as a Phenomenex Luna C18 75×30 mm×3 μm column (eluent: 48% to 78%, CH₃CN and H₂O (with 0.2% FA)). The pure fractions were collected and concentrated under vacuum. The residue was suspended in water (10 mL) and the mixture was frozen by inserting it into a −78° C. bath, and then lyophilized to yield (R,S)-7-(3-(2-(1H-pyrrolo[2,3-b]pyridin-3-yl)thiazol-4-yl)phenyl)-7H-cyclopenta[b]pyridin-7-ol (200 mg, 9%) as a yellow solid. LCMS (ESI): mass calcd. for C₂₄H₁₆N₄OS, 408.5; m/z found, 409.1 [M+H]⁺. ¹H NMR (400 MHz, DMSO-d₆) δ 8.53-8.47 (m, 1H), 8.39-8.35 (m, 1H), 8.29-8.23 (m, 2H), 8.07 (s, 1H), 7.94-7.87 (m, 2H), 7.76-7.70 (m, 1H), 7.45-7.37 (m, 2H), 7.34-7.25 (m, 2H), 6.96 (d, J=6.0 Hz, 1H), 6.63 (d, J=6.0 Hz, 1H), 6.33 (5, 1H).

Step B. (R)-7-(3-(2-(1H-pyrrolo[2,3-b]pyridin-3-yl)thiazol-4-yl)phenyl)-7H-cyclopenta[b]pyridin-7-ol. The (R) and (S) enantiomers of (R,S)-7-(3-(2-(1H-pyrrolo[2,3-b]pyridin-3-yl)thiazol-4-yl)phenyl)-7H-cyclopenta[b]pyridin-7-ol were separated by SFC using an SFC column, such as a DAICEL CHIRALPAK IC 250 mm×30 mm×10 μm column (isocratic elution: EtOH (containing 0.1% of 25% aq. NH₃): supercritical CO₂, 45%: 55%). The second eluting enantiomer, (R)-7-(3-(2-(1H-pyrrolo[2,3-b]pyridin-3-yl)thiazol-4-yl)phenyl)-7H-cyclopenta[b]pyridin-7-ol (64.4 mg, 31%) was obtained as a colorless solid. The first eluting enantiomer was (S)-7-(3-(2-(1H-pyrrolo[2,3-b]pyridin-3-yl)thiazol-4-yl)phenyl)-7H-cyclopenta[b]pyridin-7-ol (Example 34). Data for (R)-7-(3-(2-(1H-pyrrolo[2,3-b]pyridin-3-yl)thiazol-4-yl)phenyl)-7H-cyclopenta[b]pyridin-7-ol: SFC Rₜ=3.595 min. LCMS (ESI): mass calcd. for C₂₄H₁₆N₄OS, 408.5; m/z found, 409.0 [M+H]⁺. ¹H NMR (400 MHz, DMSO-d₆) δ 8.53-8.47 (m, 1H), 8.39-8.35 (m, 1H), 8.29-8.23 (m, 2H), 8.07 (s, 1H), 7.94-7.87 (m, 2H), 7.76-7.70 (m, 1H), 7.45-7.37 (m, 2H), 7.34-7.25 (m, 2H), 6.96 (d, J=6.0 Hz, 1H), 6.63 (d, J=6.0 Hz, 1H), 6.33 (s, 1H).

### Example 34. (S)-7-(3-(2-(1H-Pyrrolo[2,3-b]pyridin-3-yl)thiazol-4-yl)phenyl)-7H-cyclopenta[b]pyridin-7-ol

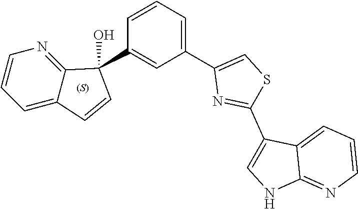

The chiral separation from Example 33 provided (S)-7-(3-(2-(1H-pyrrolo[2,3-b]pyridin-3-yl)thiazol-4-yl)phenyl)-7H-cyclopenta[b]pyridin-7-oi, (73.7 mg, 35%). SFC Rₜ=2.998 min. LCMS (ESI): mass calcd. for C₂₄H₁₆N₄OS, 408.5; m/z found, 409.0 [M+H]⁺. ¹H NMR (400 MHz, DMSO-d₅) S 12.29 (br s, 1H), 8.54-8.47 (m, 1H), 8.40-8.34 (m, 1H), 8.28-8.23 (m, 2H), 8.07 (s, 1H), 7.93-7.87 (m, 2H), 7.76-7.70 (m, 1H), 7.45-7.36 (m, 2H), 7.35-7.23 (m, 2H), 6.96 (d, J=6.0 Hz, 1H), 6.63 (d, J=6.0 Hz, 1H), 6.32 (s, 1H).

### Example 35. (S)-7-(3-(2-(1H-Pyrrolo[2,3-b]pyridin-3-yl)thiazol-4-yl)phenyl)-6,6-dimethyl-6,7-dihydro-5H-cyclopenta[b]pyridin-7-ol

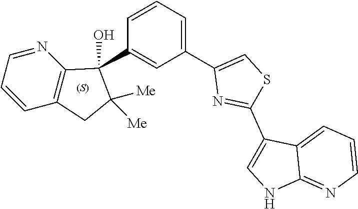

Step A. (R,S)-7-(3-(2-(1H-Pyrrolo[2,3-b]pyridin-3-yl)thiazol-4-yl)phenyl)-6,6-dimethyl-6,7-dihydro-5H-cyclopenta[b]pyridin-7-ol. Sodium hydroxide (3.5 mL, 2.0 M in H₂O, 7.0 mmol) was added to a mixture of (R,S)-6,6-dimethyl-7-(3-(2-(1-(phenylsulfonyl)-1H-pyrrolo[2,3-b]pyridin-3-yl)thiazol-4-yl)phenyl)-6,7-dihydro-5H-cyclopenta[b]pyridin-7-ol (Intermediate 68, 2.0 g, 2.2 mmol) and 1,4-dioxane (10 mL). The resultant mixture was heated at 60° C. for 2 h, then cooled to room temperature, concentrated under reduced pressure, and purified by preparative HPLC using an HPLC column, such as a YMC-Triart Prep C18 150×40 mm×7 μm column (eluent: 57% to 67%, CH₃CN and H₂O (with 0.04% NH₃·H₂O+10 mM NH₄HCO₃)) to yield the product. The product was suspended in water (10 mL) and the mixture was frozen by inserting it into a −78° C. bath, and then lyophilized to yield (R,S)-7-(3-(2-(1H-pyrrolo[2,3-b]pyridin-3-yl)thiazol-4-yl)phenyl)-6,6-dimethyl-6,7-dihydro-5H-cyclopenta[b]pyridin-7-ol (400 mg, 41%) as a colorless solid. LCMS (ESI): mass calcd. for C₂₆H₂₂N₄OS, 438.15; m/z found, 439.2 [M+H]⁺.

Step B. (S)-7-(3-(2-(1H-pyrrolo[2,3-b]pyridin-3-yl)thiazol-4-yl)phenyl)-6,6-dimethyl-6,7-dihydro-5H-cyclopenta[b]pyridin-7-ol. The enantiomers of (R,S)-7-(3-(2 (1H-Pyrrolo[2,3-b]pyridin-3-yl)thiazol-4-yl)phenyl)-6,6-dimethyl-6,7-dihydro-5H-cyclopenta[b]pyridin-7-ol (140 mg, 0.319 mmol) were separated by SFC using an SFC column, such as a DAICEL CHIRALCEL OJ-H 250 mm×30 mm×5 μm column (isocratic elution: ethanol (containing 0.1% of 25% aqueous NH₃): supercritical CO₂, 10%: 90%) to provide the first eluting enantiomer, (S)-7-(3-(2-(1H-pyrrolo[2,3-b]pyridin-3-yl)thiazol-4-yl)phenyl)-6,6-dimethyl-6,7-dihydro-5H-cyclopenta[b]pyridin-7-ol, (SFC Rₜ=1.680 min) and the second eluting enantiomer, (R)-7-(3-(2-(1H-pyrrolo[2,3-b]pyridin-3-yl)thiazol-4-yl)phenyl)-6,6-dimethyl-6,7-dihydro-5H-cyclopenta[b]pyridin-7-ol (Example 36).

### Example 36. (R)-7-(3-(2-(1H-Pyrrolo[2,3-b]pyridin-3-yl)thiazol-4-yl)phenyl)-6,6-dimethyl-6,7-dihydro-5H-cyclopenta[b]pyridin-7-ol

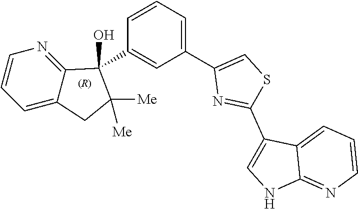

The chiral separation from Example 35 also provided (R)-7-(3-(2-(1H-pyrrolo[2,3-b]pyridin-3-yl)thiazol-4-yl)phenyl)-6,6-dimethyl-6,7-dihydro-5H-cyclopenta[b]pyridin-7-ol, SFC Rₜ=2.257 min. (23.7 mg, 16%) as a colorless solid. LCMS (ESI): mass calcd. for C₂₆H₂₂N₄OS, 438.15; m/z found, 439.1 [M+H]⁺. ¹H NMR (400 MHz, CD₃OD) δ 8.66-8.60 (m, 1H), 8.50-8.43 (m, 1H), 8.37-8.31 (m, 1H), 8.07 (s, 1H), 7.97-7.91 (m, 2H), 7.90-7.83 (m, 1H), 7.62 (5, 1H), 7.50-7.39 (m, 2H), 7.37-7.26 (m, 2H), 3.08-2.98 (m, 1H), 2.88-2.78 (m, 1H), 1.28 (s, 3H), 0.77 (s, 3H).

### Example 37. (S)-8-(3-(2-(1H-Pyrrolo[2,3-b]pyridin-3-yl)thiazol-4-yl)phenyl)-5,6,7,8-tetrahydroimidazo[1,2-a]pyridin-8-ol

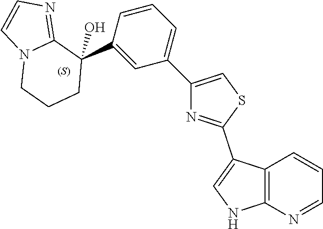

Step A. (R,S)-8-(3-(2-(1H-pyrrolo[2,3-b]pyridin-3-yl)thiazol-4-yl)phenyl)-5,6,7,8-tetrahydroimidazo[1,2-a]pyridin-8-ol. Sodium hydroxide (4 mL, 2.0 M in H₂O, 8 mmol) was added to a solution of (R,S)-8-(3-(2-(1-(phenylsulfonyl)-1H-pyrrolo[2,3-b]pyridin-3-yl)thiazol-4-yl)phenyl)-5,6,7,8-tetrahydroimidazo[1,2-a]pyridin-8-ol (Intermediate 70, 910 mg, 1.6 mmol) and 1,4-dioxane (20 mL). The resultant mixture was heated at 60° C. for 2 h. The reaction mixture was cooled to room temperature, concentrated, and were partially purified by preparative HPLC using an HPLC column, such as a spherical C18 20-35 μm column (eluent: 5% to 100%, ACN and H₂O), and then tritutated with petroleum ether/ethyl acetate (40 mL, 1:10) to yield (R,S)-8-(3-(2-(1H-pyrrolo[2,3-b]pyridin-3-yl)thiazol-4-yl)phenyl)-5,6,7,8-tetrahydroimidazo[1,2-a]pyridin-8-ol (450 mg, 62%) as a yellow solid. LCMS (ESI): mass calcd. for C₂₃H₁₉N₅OS, 413.13; m/z found, 414.4 [M+H]⁺.

Step B. (S)-8-(3-(2-(1H-Pyrrolo[2,3-b]pyridin-3-yl)thiazol-4-yl)phenyl)-5,6,7,8-tetrahydroimidazo[1,2-a]pyridin-8-ol. The enantionmers of (R,S)-8-(3-(2-(1H-pyrrolo[2,3-b]pyridin-3-yl)thiazol-4-yl)phenyl)-5,6,7,8-tetrahydroimidazo[1,2-a]pyridin-8-ol (450 mg) were separated by SFC using an SFC column, such as a DAICEL CHIRALCEL AS 250 mm×30 mm×10 μm column (isocratic elution: EtOH (containing 0.1% of 25% aqueous NH₃): supercritical CO₂, 40%: 60%) to yield the first eluting enantiomer (S)-8-(3-(2-(1H-pyrrolo[2,3-b]pyridin-3-yl)thiazol-4-yl)phenyl)-5,6,7,8-tetrahydroimidazo[1,2-a]pyridin-8-ol and a second eluting enantiomer (R)-8-(3-(2-(1H-pyrrolo[2,3-b]pyridin-3-yl)thiazol-4-yl)phenyl)-5,6,7,8-tetrahydroimidazo[1,2-a]pyridin-8-ol (Example 38). The (S)-8-(3-(2 (1H-pyrrolo[2,3-b]pyridin-3-yl)thiazol-4-yl)phenyl)-5,6,7,8-tetrahydroimidazo[1,2-a]pyridin-8-ol was suspended in water (10 mL) and the mixture was frozen by inserting it into a −78° C. bath, and then lyophilized to yield (S)-8-(3-(2-(1H-pyrrolo[2,3-b]pyridin-3-yl)thiazol-4-yl)phenyl)-5,6,7,8-tetrahydroimidazo[1,2-a]pyridin-8-ol, SFC Rₜ=3.609 min. LCMS (ESI): mass calcd. for C₂₃H₁₉N₅OS, 413.13; m/z found, 414.0 [M+H]⁺. ¹H NMR (400 MHz, DMSO-d₆) δ 12.28 (br s, 1H), 8.59 (dd, J=1.5, 8.0 Hz, 1H), 8.36 (dd, J=1.4, 4.6 Hz, 1H), 8.26 (s, 1H), 8.01-7.98 (m, 1H), 7.93-7.89 (m, 1H), 7.87 (s, 1H), 7.43-7.37 (m, 1H), 7.33-7.27 (m, 2H), 7.15 (d, J=0.8 Hz, 1H), 6.93 (s, 1H), 5.93 (s, 1H), 4.19-4.01 (m, 2H), 2.30-2.09 (m, 3H), 1.90-1.79 (m, 1H).

### Example 38. (R)-8-(3-(2-(1H-Pyrrolo[2,3-b]pyridin-3-yl)thiazol-4-yl)phenyl)-5,6,7,8-tetrahydroimidazo[1,2-a]pyridin-8-ol

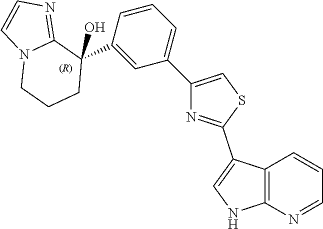

The chiral separation from Example 37 provided (R)-8-(3-(2-(1H-pyrrolo[2,3-b]pyridin-3-yl)thiazol-4-yl)phenyl)-5,6,7,8-tetrahydroimidazo[1,2-a]pyridin-8-ol. This product was suspended in water (10 mL), the mixture was frozen by inserting it into a −78° C. bath, and then lyophilized to yield (R)-8-(3-(2-(1H-pyrrolo[2,3-b]pyridin-3-yl)thiazol-4-yl)phenyl)-5,6,7,8-tetrahydroimidazo[1,2-a]pyridin-8-ol (100 mg, 24%) as a yellow solid. SFC Rₜ=3.878 min. LCMS (ESI): mass calcd. for C₂₃H₁₉N₅OS, 413.13; m/z found, 414.0 [M+H]⁺. ¹H NMR (400 MHz, DMSO-d₆) δ 12.27 (br s, 1H), 8.59 (dd, J=7.8, 1.5 Hz, 1H), 8.36 (dd, J=4.5, 1.5 Hz, 1H), 8.26 (s, 1H), 8.03-7.96 (m, 1H), 7.94-7.89 (m, 1H), 7.87 (s, 1H), 7.43-7.36 (m, 1H), 7.33-7.27 (m, 2H), 7.15 (d, J=0.8 Hz, 1H), 6.93 (s, 1H), 5.96-5.91 (m, 1H), 4.19-4.01 (m, 2H), 2.29-2.09 (m, 3H), 1.90-1.78 (m, 1H).

### Example 39. (S)-3-(3-(2-(5H-Pyrrolo[2,3-b]pyrazin-7-yl)thiazol-4-yl)phenyl)-3-hydroxy-1-methylpyrrolidin-2-one

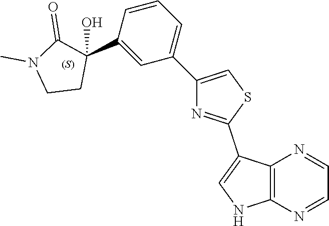

Step A. (R,S)-3-(3-(2-(5H-Pyrrolo[2,3-b]pyrazin-7-yl)thiazol-4-yl)phenyl)-3-hydroxy-1-methylpyrrolidin-2-one. NaOH (1.92 mL, 2.0 M in water, 3.84 mmol) was added to a solution of 3-hydroxy-1-methyl-3-(3-(2-(5-tosyl-5H-pyrrolo[2,3-b]pyrazin-7-yl)thiazol-4-yl)phenyl)pyrrolidin-2-one, (Intermediate 71, 350 mg, 0.64 mmol) and 1,4 dioxane (5 mL). The resultant mixture was heated at 60° C. for 1 h, cooled to room temperature and concentrated to dryness under reduced pressure. The resulting residue was re-dissolved in water (10 mL), and the pH was adjusted to pH=6-7 with 1 N HCl. This solution was then extracted with dichloromethane:MeOH (10:1, 20 mL×3). The combined organic solvent extracts were dried over anhydrous Na₂SO₄, filtered, and concentrated to yield (R,S)-3-(3-(2-(5H-pyrrolo[2,3-b]pyrazin-7-yl)thiazol-4-yl)phenyl)-3-hydroxy-1-methylpyrrolidin-2-one (230 mg, 86%) as a brown solid. LCMS (ESI): mass calcd. for C₂₀H₁₇N₅O₂S, 391.11: m/z found, 392.1 [M+1]⁺.

Step B. (S)-3-(3-(2-(5H-Pyrrolo[2,3-b]pyrazin-7-yl)thiazol-4-yl)phenyl)-3-hydroxy-1-methylpyrrolidin-2-one. The enantiomers of (R,S)-3-(3-(2-(5H-Pyrrolo[2,3-b]pyrazin-7-yl)thiazol-4-yl)phenyl)-3-hydroxy-1-methylpyrrolidin-2-one (230 mg, 0.59 mmol) were separated by SFC using an SFC column, such as a DAICEL CHIRALPAK AS-H 250 mm×30 mm, 5 μm column (isocratic elution: EtOH (containing 0.1% of 25% aqueous NH₃): supercritical CO₂, 50%: 50%) to yield (S)-3-(3-(2-(5H-Pyrrolo[2,3-b]pyrazin-7-yl)thiazol-4-yl)phenyl)-3-hydroxy-1-methylpyrrolidin-2-one as the first eluting enantiomer and (R)-3 (3-(2-(5H-Pyrrolo[2,3-b]pyrazin-7-yl)thiazol-4-yl)phenyl)-3-hydroxy-1-methylpyrrolidin-2-one as a second eluting enantiomer (Example 40). The pure fractions of (S)-3-(3-(2-(5H-pyrrolo[2,3-b]pyrazin-7-yl)thiazol-4-yl)phenyl)-3-hydroxy-1-methylpyrrolidin-2-one were collected and concentrated under reduced pressure. This product was suspended in water (10 mL) and the mixture was frozen by inserting it into a −78° C. bath, and then lyophilized to yield (S)-3-(3-(2-(5H-pyrrolo[2,3-b]pyrazin-7-yl)thiazol-4-yl)phenyl)-3-hydroxy-1-methylpyrrolidin-2-one (89.6 mg, 38%) as a yellow solid. SFC Rₜ=3.982 min. LCMS (ESI): mass calcd. for C₂₀H₁₇N₅O₂S, 391.11; m/z found, 392.0 [M+H]⁺. ¹H NMR (400 MHz, DMSO-d₆) δ 12.67 (br s, 1H), 8.63 (s, 1H), 8.60 (d, J=2.4 Hz, 1H), 8.40 (d, J=2.4 Hz, 1H), 8.10-8.06 (m, 1H), 8.04 (s, 1H), 7.96 (d, J=7.6 Hz, 1H), 7.46-7.40 (m, 1H), 7.33 (d, J=7.8 Hz, 1H), 6.09 (br s, 1H), 3.51-3.44 (m, 1H), 3.43-3.38 (m, 1H), 2.87 (s, 3H), 2.44-2.35 (m, 1H), 2.34-2.24 (m, 1H).

### Example 40. (R)-3-(3-(2-(5H-Pyrrolo[2,3-b]pyrazin-7-yl)thiazol-4-yl)phenyl)-3-hydroxy-1-methylpyrrolidin-2-one

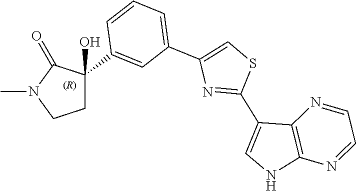

The chiral separation from Example 39, Step B yielded (R)-3-(3-(2-(5H-pyrrolo[2,3-b]pyrazin-7-yl)thiazol-4-yl)phenyl)-3-hydroxy-1-methylpyrrolidin-2-one (78.8 mg, 34%) as a yellow solid. SFC Rₜ=4.687 min. LCMS (ESI): mass calcd. for C₂₀H₁₇N₅O₂S, 391.11; m/z found, 392.0 [M+H]⁺. ¹H NMR (400 MHz, DMSO-d₆) δ 8.63 (s, 1H), 8.59 (d, J=2.7 Hz, 1H), 8.40 (d, J=2.4 Hz, 1H), 8.10-8.06 (m, 1H), 8.03 (s, 1H), 7.96 (d, J=7.8 Hz, 1H), 7.46-7.40 (m, 1H), 7.33 (d, J=7.8 Hz, 1H), 6.10 (br s, 1H), 3.51-3.44 (m, 1H), 3.43-3.36 (m, 1H), 2.87 (s, 3H), 2.44-2.35 (m, 1H), 2.33-2.24 (m, 1H).

### Example 41. (R)-7-(3-(2-(5H-Pyrrolo[2,3-b]pyrazin-7-yl)thiazol-4-yl)phenyl)-6,7-dihydro-5H-pyrrolo[1,2-a]imidazol-7-ol

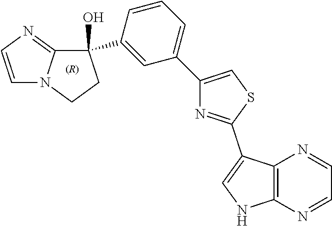

(R)-7-(3-(2-(5-Tosyl-5H-pyrrolo[2,3-b]pyrazin-7-yl)thiazol-4-yl)phenyl)-6,7-dihydro-5H-pyrrolo[1,2-a]imidazol-7-ol (Intermediate 14, 300 mg, 0.54 mmol) was added to a mixture of aqueous NaOH (1.6 mL, 2 M in water, 3.2 mmol) and 1,4-dioxane (6 mL). The reaction mixture was heated at 60° C. for 2 h, cooled to room temperature, and the pH was adjusted to pH=7 with 6 M HCl. The reaction mixture was concentrated to dryness under reduced pressure and purified by preparative HPLC using an HPLC column, such as an Xtimate C18 250 mm×50 mm×10 μm column (eluent: 30% to 60%, CH₃CN and H₂O (with 0.04% NH₄OH+10 mM NH₄HCO₃)). The product was suspended in water (10 mL) and the mixture was frozen by inserting it into a −78° C. bath, and then lyophilized to yield (R)-7-(3-(2-(5H-pyrrolo[2,3-b]pyrazin-7-yl)thiazol-4-yl)phenyl)-6,7-dihydro-5H-pyrrolo[1,2-a]imidazol-7-ol (97.5 mg, 45%) as a white solid. LCMS(ESI): mass calcd. for C₂₁H₁₆N₆OS, 400.11: m/z found, 401.1 [M+H]⁺. ¹H NMR (400 MHz, DMSO-d₆) δ 12.79 (br s, 1H), 8.62 (s, 2H), 8.43 (d, J=2.5 Hz, 1H), 8.20 (s, 1H), 8.03 (s, 1H), 7.99-7.95 (m, 1H), 7.47-7.40 (m, 2H), 7.22-7.18 (m, 1H), 7.03 (s, 1H), 6.18 (s, 1H), 4.22-4.05 (m, 2H), 2.95-2.81 (m, 2H).

### Example 42. (S)-7-(3-(2-(5H-Pyrrolo[2,3-b]pyrazin-7-yl)thiazol-4-yl)phenyl)-6,7-dihydro-5H-pyrrolo[1,2-a]imidazol-7-ol

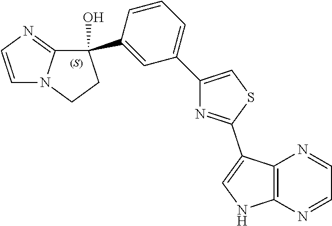

Aqueous NaOH (0.54 mL, 2.0 M, 1.08 mmol) was added to a solution of (S)-7-(3-(2-(5-tosyl-5H-pyrrolo[2,3-b]pyrazin-7-yl)thiazol-4-yl)phenyl)-6,7-dihydro-5H-pyrrolo[1,2-a]imidazol-7-ol (Intermediate 119, 85 mg, 0.15 mmol) and 1,4-dioxane (2 mL). The resultant mixture was heated at 60° C. for 1 h, cooled to room temperature, and concentrated to dryness under reduced pressure. The material was re-dissolved in water (10 mL), the pH of the resultant mixture was adjusted to pH=6-7 with 1 N HCl and extracted with ethyl acetate (20 mL×3). The combined organic solvent extracts were dried over anhydrous Na₂SO₄, filtered, and concentrated to dryness under reduced pressure to yield the product, which was purified by preparative HPLC using an HPLC column, such as an Xtimate C18 250 mm×50 mm, 10 μm column (eluent: 38% to 68%, CH₃CN and H₂O (with 0.04% NH₃)). The product was suspended in water (10 mL) and the mixture was frozen by inserting it into a −78° C. bath, and then lyophilized to yield (S)-7-(3-(2-(5H-pyrrolo[2,3-b]pyrazin-7-yl)thiazol-4-yl)phenyl)-6,7-dihydro-5H-pyrrolo[1,2-a]imidazol-7-ol (10.2 mg, 17%) as a yellow solid. LCMS(ESI): mass calcd. for C₂₁H₁₆N₆OS, 400.11; m/z found, 401.1 [M+H]⁺. ¹H NMR (400 MHz, DMSO-d₆) δ 12.78 (br s, 1H), 8.62 (s, 2H), 8.42 (d, J=2.4 Hz, 1H), 8.19 (s, 1H), 8.02 (s, 1H), 7.98-7.93 (m, 1H), 7.46-7.38 (m, 2H), 7.19 (s, 1H), 7.02 (s, 1H), 6.17 (s, 1H), 4.22-4.05 (m, 2H), 2.95-2.78 (m, 2H).

### Example 43. (S)-3-(3-(2-(5H-Pyrrolo[2,3-b]pyrazin-7-yl)thiazol-4-yl)phenyl)-3-hydroxy-1-(2,2,2-trifluoroethyl)pyrrolidin-2-one

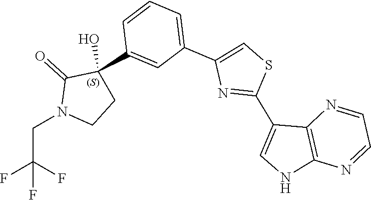

(S)-3-Hydroxy-3-(3-(2-(5-tosyl-5H-pyrrolo[2,3-b]pyrazin-7-yl)thiazol-4-yl)phenyl)-1-(2,2,2-trifluoroethyl)pyrrolidin-2-one (Intermediate 118, 140 mg, 0.23 mmol) was added to a solution of aqueous NaOH (0.68 mL, 2 M in water, 1.36 mmol) and 1,4-dioxane (6 mL). The reaction mixture was heated at 60° C. for 1.5 h. The reaction mixture was cooled to room temperature and the pH was adjusted to pH=7 with 6 M HCl. The reaction mixture was concentrated to dryness under reduced pressure and purified by preparative HPLC using an HPLC column, such as a Xtimate C18 250 mm×50 mm×10 μm column (eluent: 38% to 68%, CH₃CN and H₂O (with 0.04% NH₄OH+10 mM NH₄HCO₃)). The product was suspended in water (10 mL) and the mixture was frozen by inserting it into a −78° C. bath, and then lyophilized to yield (S)-3-(3-(2-(5H-pyrrolo[2,3-b]pyrazin-7-yl)thiazol-4-yl)phenyl)-3-hydroxy-1-(2,2,2-trifluoroethyl)pyrrolidin-2-one (43.5 mg, 41%) as a yellow solid. LCMS(ESI): mass calcd. for C₂₁H₁₆F₃N₅O₂S, 459.10; m/z found, 460.1 [M+H]⁺. ¹H NMR (400 MHz, DMSO-d₆) δ 12.81 (br s, 1H), 8.63 (d, J=2.5 Hz, 1H), 8.61 (s, 1H), 8.43 (d, J=2.8 Hz, 1H), 8.12-8.09 (m, 1H), 8.05 (s, 1H), 7.99 (d, J=8.0 Hz, 1H), 7.50-7.44 (m, 1H), 7.36 (d, J=7.8 Hz, 1H), 6.34 (s, 1H), 4.37-4.12 (m, 2H), 3.67-3.48 (m, 2H), 2.48-2.43 (m, 1H), 2.40-2.33 (m, 1H).

### Example 44. (R)-3-(3-(2-(5H-Pyrrolo[2,3-b]pyrazin-7-yl)thiazol-4-yl)phenyl)-3-hydroxy-1-(2,2,2-trifluoroethyl)pyrrolidin-2-one

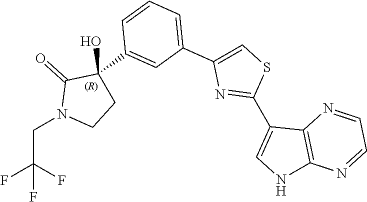

Aqueous NaOH (0.54 mL, 2.0 M in water, 1.08 mmol) was added to a solution of (R)-3-hydroxy-3-(3-(2-(5-tosyl-5H-pyrrolo[2,3-b]pyrazin-7-yl)thiazol-4-yl)phenyl)-1-(2,2,2-trifluoroethyl)pyrrolidin-2-one (Intermediate 79, 110 mg, 0.18 mmol) and 1,4-dioxane (5 mL). The resultant mixture was heated at 60° C. for 1 h, cooled to room temperature, concentrated to dryness, and purified by preparative HPLC using an HPLC column, such as a Boston Prime C18 150 mm×30 mm×5 μm column (eluent: 30% to 60%, CH₃CN and H₂O (with 0.04% NH₄OH+10 mM NH₄HCO₃)) to yield the product. The product was suspended in water (10 mL) and the mixture was frozen by inserting it into a −78° C. bath, and then lyophilized to yield (R)-3-(3-(2-(5H-pyrrolo[2,3-b]pyrazin-7-yl)thiazol-4-yl)phenyl)-3-hydroxy-1-(2,2,2-trifluoroethyl)pyrrolidin-2-one (41.6 mg, 51%) as a white solid. LCMS (ESI): mass calcd. for C₂₁H₁₆F₃N₅O₂S, 459.10; m/z found, 460.1 [M+H]⁺. ¹H NMR (400 MHz, DMSO-d₆) δ 11.03 (br s, 1H), 8.64-8.56 (m, 2H), 8.41 (d, J=2.4 Hz, 1H), 8.12-8.07 (m, 1H), 8.04 (s, 1H), 7.98 (d, J=7.7 Hz, 1H), 7.49-7.43 (m, 1H), 7.35 (d, J=7.9 Hz, 1H), 6.33 (br s, 1H), 4.38-4.07 (m, 2H), 3.68-3.45 (m, 2H), 2.47-2.41 (m, 1H), 2.40-2.31 (m, 1H).

### Example 45. (S)-3-(3-(2-(5H-Pyrrolo[2,3-b]pyrazin-7-yl)thiazol-4-yl)phenyl)-3-hydroxy-1-methylazetidin-2-one

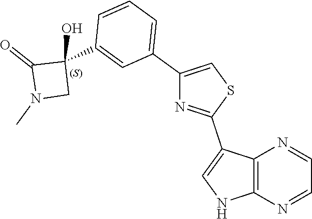

(S)-1-Methyl-2-oxo-3-(3-(2-(5-tosyl-5H-pyrrolo[2,3-b]pyrazin-7-yl)thiazol-4-yl)phenyl)azetidin-3-yl acetate (Intermediate 88, 100 mg, 0.17 mmol) was added to a mixture of NaOH (0.52 mL, 2 M in water, 1.0 mmol) and 1,4-dioxane (3 mL). The reaction mixture was stirred at room temperature for 4 h, and then the pH was adjusted to pH=7 with 1M HCl. The reaction mixture was concentrated to dryness and purified by preparative HPLC using an HPLC column, such as an Xtimate C18 250 mm×50 mm×10 μm column (eluent: 25% to 55%, CH₃CN and H₂O (with 0.04% NH₄OH+10 mM NH₄HCO₃)). The product was suspended in water (10 mL) and the mixture was frozen by inserting it into a −78° C. bath, and then lyophilized to yield (S)-3-(3-(2-(5H-pyrrolo[2,3-b]pyrazin-7-yl)thiazol-4-yl)phenyl)-3-hydroxy-1-methylazetidin-2-one (23.1 mg, 35%) as a yellow solid. LCMS(ESI): mass calcd. for C₁₉H₁₅N₅O₂S, 377.09; m/z found, 378.1 [M+H]⁺. ¹H NMR (400 MHz, DMSO-d₆) δ 12.80 (br s, 1H), 8.66 (s, 1H), 8.62 (d, J=2.5 Hz, 1H), 8.43 (d, J=2.5 Hz, 1H), 8.16-8.13 (m, 1H), 8.10 (s, 1H), 8.03-7.99 (m, 1H), 7.51-7.46 (m, 1H), 7.45-7.41 (m, 1H), 6.85 (s, 1H), 3.66 (d, J=5.5 Hz, 1H), 3.52 (d, J=5.5 Hz, 1H), 2.91 (s, 3H).

### Example 46. (R)-3-(3-(2-(5H-Pyrrolo[2,3-b]pyrazin-7-yl)thiazol-4-yl)phenyl)-3-hydroxy-1-methylazetidin-2-one

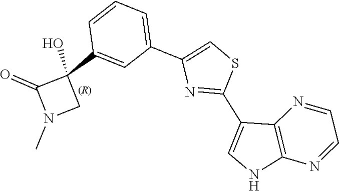

(R)-1-Methyl-2-oxo-3-(3-(2-(5-tosyl-5H-pyrrolo[2,3-b]pyrazin-7-yl)thiazol-4-yl)phenyl)azetidin-3-yl acetate (Intermediate 89, 100 mg, 0.17 mmol) was added to a mixture of NaOH (0.52 mL, 2 M in water, 1.0 mmol) and 1,4-dioxane (3 mL). The reaction mixture was stirred at room temperature for 4 h, and then the pH was adjusted to pH=7 with 1M HCl. The reaction mixture was concentrated to dryness and purified by preparative HPLC using an HPLC column, such as an Xtimate C18 250 mm×50 mm×10μm column (eluent: 25% to 55%, CH₃CN and H₂O (with 0.04% NH₄OH+10 mM NH₄HCO₃)). The product was suspended in water (10 mL) and the mixture was frozen by inserting it into a −78° C. bath, and then lyophilized to yield (R)-3-(3-(2-(5H-pyrrolo[2,3-b]pyrazin-7-yl)thiazol-4-yl)phenyl)-3-hydroxy-1-methylazetidin-2-one (31.0 mg, 47%) as a yellow solid. LCMS(ESI): mass calcd. for C₁₉H₁₅N₅O₂S, 377.09; m/z found, 378.1 [M+H]⁺. ¹H NMR (400 MHz, DMSO-d₆) δ 8.65 (s, 1H), 8.61 (d, J=2.5 Hz, 1H), 8.41 (d, J=2.5 Hz, 1H), 8.15-8.13 (m, 1H), 8.09 (s, 1H), 8.03-7.99 (m, 1H), 7.51-7.46 (m, 1H), 7.45-7.41 (m, 1H), 6.87 (br s, 1H), 3.66 (d, J=5.5 Hz, 1H), 3.52 (d, J=5.5 Hz, 1H), 2.91 (s, 3H).

### Example 47. (R)-3-(3-(2-(1H-Pyrrolo[2,3-b]pyridin-3-yl)thiazol-4-yl)phenyl)-3-hydroxy-1-methylpyrrolidin-2-one

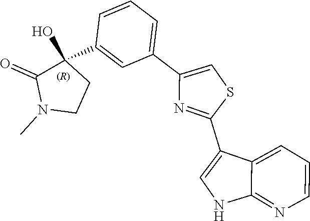

Step A. (R,S)-3-(3-(2-(1H-pyrrolo[2,3-b]pyridin-3-yl)thiazol-4-yl)phenyl)-3-hydroxy-1-methylpyrrolidin-2-one. To a flask containing (R,S)-3-hydroxy-1-methyl-3-(3-(2-(1-(phenylsulfonyl)-1H-pyrrolo[2,3-b]pyridin-3-yl)thiazol-4-yl)phenyl)pyrrolidin-2-one, (Intermediate 90, 250 mg, 0.47 mmol) was added THE (22 mL) and LiOH (85 mg, 3.34 mmol in 3 mL water). The mixture was placed in an aluminum mantle, that had been pre-heated at 40° C., and allowed to stir over night. After 17 h, the reaction mixture was filtered through a pad of diatomaceous earth and the filtrate was concentrated to a yellow gum. This material was re-dissolved in THE and diatomaceous earth (2.5 g) was added and the reaction mixture was concentrated to dryness. The material was purified by FCC (using 100% DCM increasing to 10% MeOH-DCM) to yield (R,S)-3-(3-(2-(1H-pyrrolo[2,3-b]pyridin-3-yl)thiazol-4-yl)phenyl)-3-hydroxy-1-methylpyrrolidin-2-one (162 mg, 88%) as an off-white solid. MS (ESI): mass calcd. for C₂₁H₁₈N₄O₂S, 390.47; m/z found, 391.10 [M+H]⁺. ¹H NMR (400 MHz, CDCl₃) δ 11.08 (s, 1H), 8.54 (dd, J=7.9, 1.5 Hz, 1H), 8.40-8.33 (m, 1H), 7.94-7.85 (m, 1H), 7.45-7.28 (m, 3H), 7.21-7.10 (m, 2H), 6.66 (s, 1H), 3.88-3.60 (m, 1H), 3.60-3.43 (m, 1H), 3.12 (s, 3H), 2.64-2.29 (m, 2H).

Step B. (R)-3-(3-(2-(1H-pyrrolo[2,3-b]pyridin-3-yl)thiazol-4-yl)phenyl)-3-hydroxy-1-methylpyrrolidin-2-one. The enantiomers of (R,S)-3-(3-(2-(1H-pyrrolo[2,3-b]pyridin-3-yl)thiazol-4-yl)phenyl)-3-hydroxy-1-methylpyrrolidin-2-one were separated by SFC using an SFC column, such as a Lux Cellulose 1 column, 5 μm, 250×21 mm, Mobile phase: 25% methanol:isopropanol (1:1) with 0.2% isopropylamine, 75% CO₂) to provide, (R)-3 (3-(2-(1H-pyrrolo[2,3-b]pyridin-3-yl)thiazol-4-yl)phenyl)-3-hydroxy-1-methylpyrrolidin-2-one (59 mg) as the first eluting enantiomer and (S)-3-(3-(2-(1H-pyrrolo[2,3-b]pyridin-3-yl)thiazol-4-yl)phenyl)-3-hydroxy-1-methylpyrrolidin-2-one (Example 48) as the second eluting enantiomer. Data for (R)-3-(3-(2-(1H-pyrrolo[2,3-b]pyridin-3-yl)thiazol-4-yl)phenyl)-3-hydroxy-1-methylpyrrolidin-2-one: ¹H NMR (400 MHz, DMSO-d₆) δ 12.30 (s, 1H), 8.68 (dd, J=7.9, 1.6 Hz, 1H), 8.37 (dd, J=4.7, 1.6 Hz, 1H), 8.29 (br s, 1H), 8.09 (br s, 1H), 8.02-7.89 (m, 2H), 7.46 (t, J=7.7 Hz, 1H), 7.34-7.37 (m, 1H), 7.28-7.33 (m, 1H), 6.11 (s, 1H), 3.58-3.37 (m, 2H), 2.90 (s, 3H), 2.46-2.21 (m, 2H).

### Example 48. (S)-3-(3-(2-(1H-Pyrrolo[2,3-b]pyridin-3-yl)thiazol-4-yl)phenyl)-3-hydroxy-1-methylpyrrolidin-2-one

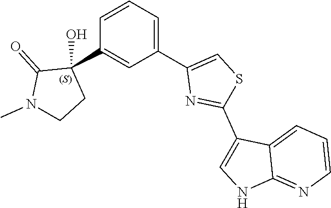

The chiral separation from Example 47 provided (S)-3-(3-(2-(1H-pyrrolo[2,3-b]pyridin-3-yl)thiazol-4-yl)phenyl)-3-hydroxy-1-methylpyrrolidin-2-one (61 mg). ¹H NMR (400 MHz, DMSO-d₆) δ 12.30 (s, 1H), 8.68 (dd, J=7.9, 1.7 Hz, 1H), 8.37 (dd, J=4.6, 1.6 Hz, 1H), 8.29 (s, 1H), 8.09 (br s, 1H), 8.02-7.81 (m, 2H), 7.46 (t, J=7.7 Hz, 1H), 7.34-7.39 (m, 1H), 7.28-7.33 (m, 1H), 6.11 (s, 1H), 3.58-3.36 (m, 2H), 2.90 (s, 3H), 2.47-2.19 (m, 2H).

### Example 49. (R)-3-Hydroxy-3-(3-(2-(5-methoxy-1H-pyrrolo[2,3-b]pyridin-3-yl)thiazol-4-yl)phenyl)-1-methylpyrrolidin-2-one

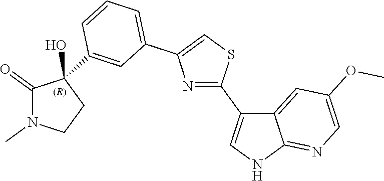

To a vial containing (R)-3-hydroxy-3-(3-(2-(5-methoxy-1-tosyl-1H-pyrrolo[2,3-b]pyridin-3-yl)thiazol-4-yl)phenyl)-1-methylpyrrolidin-2-one (Intermediate 91, 180 mg, 0.31 mmol) was added 1,4-dioxane (5 mL) followed by LiOH (60 mg, 2.51 mmol in 2 mL H₂O). The vial was sealed and placed in an aluminum mantle pre-heated at 80° C. After 1.5 h, the reaction mixture was filtered through a pad of diatomaceous earth and rinsed with THF. The filtrate was concentrated to a yellow amorphous solid which was re dissolved in THF and purified by FCC (using 100% DCM increasing to 5% MeOH-DCM) to yield (R)-3-hydroxy-3-(3-(2-(5-methoxy-1H-pyrrolo[2,3-b]pyridin-3-yl)thiazol-4-yl)phenyl)-1-methylpyrrolidin-2-one (60 mg) as an off-white solid. MS (ESI): mass calcd. for C₂₂H₂₀N₄O₃S, 420.49; m/z found, 421.10 [M+H]⁺. ¹H NMR (500 MHz, Methanol-d₄) δ 8.33 (d, J=2.8 Hz, 1H), 8.23 (t, J=1.8 Hz, 1H), 8.07 (d, J=2.8 Hz, 1H), 8.02 (s, 1H), 7.93-7.96 (m, 1H), 7.67 (s, 1H), 7.46 (t, J=7.7 Hz, 1H), 7.37-7.40 (m, 1H), 3.99 (s, 3H), 3.63-3.43 (m, 2H), 3.00 (s, 3H), 2.51-2.57 (m, 1H), 2.40-2.45 (m, 1H).

### Example 50. (R)-3-Hydroxy-3-(3-(5-(hydroxymethyl)-2-(1H-pyrrolo[2,3-b]pyridin-3-yl)thiazol-4-yl)phenyl)-1-methylpyrrolidin-2-one

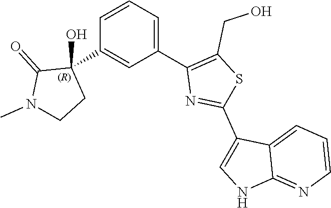

To a flask containing (R)-3-hydroxy-3-(3-(5-(hydroxymethyl)-2-(1-tosyl-1H-pyrrolo[2,3-b]pyridin-3-yl)thiazol-4-yl)phenyl)-1-methylpyrrolidin-2-one (Intermediate 92, 185 mg, 0.32 mmol) was added THF (11 mL) and LiOH (80 mg in 3 mL water). The resultant mixture was placed in an aluminum mantle that had been pre-heated at 55° C. After 3 h, the reaction mixture was cooled to room temperature and filtered through a pad of diatomaceous earth and rinsed further with THF. The filtrate was concentrated to a viscous amber gum which was re-dissolved in THF and purified by FCC (using 100% EtOAc increasing to 10% MeOH-EtOAc). This material was further purified using by HPLC using an HPLC column, such as a AccuPrep C18 50×100 mm column, CH₃CN—H₂O (0% to 100%) containing 0.05% NH₄OH to yield (R)-3-hydroxy-3-(3-(5-(hydroxymethyl)-2-(1H-pyrrolo[2,3-b]pyridin-3-yl)thiazol-4-yl)phenyl)-1-methylpyrrolidin-2-one (69 mg) as a white solid. MS (ESI): mass calcd. for C₂₂H₂₀N₄O₃S, 420.49; m/z found, 421.0 [M+H]⁺. ¹H NMR (500 MHz, Methanol-d₄) δ 8.69-8.58 (m, 1H), 8.31 (br s, 1H), 8.07 (s, 1H), 7.82 (br s, 1H), 7.68-7.74 (m, 1H), 7.48-7.60 (m, 2H), 7.26-7.32 (m, 1H), 3.66-3.42 (m, 2H), 2.99 (s, 3H), 2.50-2.58 (m, 1H), 2.40-2.46 (m, 1H).

### Example 51. (R)-3-(4-(3-(3-Hydroxy-1-methyl-2-oxopyrrolidin-3-yl)phenyl)thiazol-2-yl)-1H-pyrrolo[2,3-b]pyridine-5-carboxylic acid

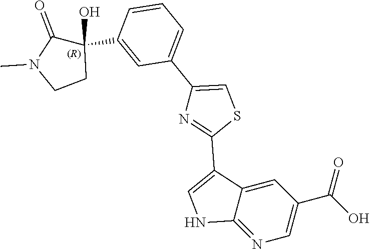

To a vial containing methyl (R)-3-(4-(3-(3-hydroxy-1-methyl-2-oxopyrrolidin-3-yl)phenyl)thiazol-2-yl)-1-tosyl-1H-pyrrolo[2,3-b]pyridine-5-carboxylate (Intermediate 93, 280 mg, 0.46 mmol) was added 1,4-dioxane (7 mL) followed by LiOH (95 mg in 3 mL H₂O). The vial was sealed and placed in an aluminum mantle that had been pre-heated at 50° C. After 5 h, the reaction mixture was filtered through a pad of diatomaceous earth which was rinsed further with THF and the filtrate was concentrated to a light brown amorphous solid. This material was purified by FCC (using 100% EtOAc increasing to 20% MeOH-EtOAc, wherein the EtOAc contained 2% HOAc) and then further purified by HPLC to the yield (R)-3-(4-(3-(3-hydroxy-1-methyl-2-oxopyrrolidin-3-yl)phenyl)thiazol-2-yl)-1H-pyrrolo[2,3-b]pyridine-5-carboxylic acid (14 mg, 4%) as a light yellow solid. ¹H NMR (400 MHz, Methanol-d₄) δ 9.36 (s, 1H), 9.03 (br 5, 1H), 8.13-8.20 (m, 2H), 7.95-7.99 (m, 1H), 7.73 (s, 1H), 7.57-7.36 (m, 2H), 3.58-3.64 (m, 2H), 3.00 (s, 3H), 2.58-2.68 (m, 1H), 2.40-2.49 (m, 1H).

### Example 52. (S)-3-(3-(2-(7H-Pyrrolo[2,3-d]pyrimidin-5-yl)thiazol-4-yl)phenyl)-3-hydroxy-1-methylpyrrolidin-2-one

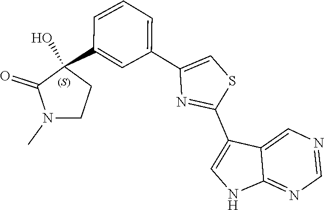

Step A. (R,S)-3-(3-(2-(7H-pyrrolo[2,3-d]pyrimidin-5-yl)thiazol-4-yl)phenyl)-3-hydroxy-1-methylpyrrolidin-2-one. To a vial containing (R,S)-3-hydroxy-1-methyl-3-(3-(2-(7-tosyl-7H-pyrrolo[2,3-d]pyrimidin-5-yl)thiazol-4-yl)phenyl)pyrrolidin-2-one (Intermediate 94, 180 mg, 0.33 mmol) was added THF (15 mL) and LiOH (100 mg in 3 mL water). The mixture was placed in an aluminum mantle that had been pre-heated at 50° C. After 1.5 h, the reaction mixture was filtered through a pad of diatomaceous earth (while still warm) and rinsed further with THF and the filtrate was concentrated to a light amber gum. This material was re-dissolved in THF and purified by FCC (using 100% DCM increasing to 10% MeOH-DCM) to yield (R,S)-3-(3-(2-(7H-pyrrolo[2,3-d]pyrimidin-5-yl)thiazol-4-yl)phenyl)-3-hydroxy-1-methylpyrrolidin-2-one (126 mg) as an off-white amorphous solid. MS (ESI): mass calcd. for C₂₀H₁₇N₅O₂S, 391.45; m/z found, 392.1 [M+H]⁺. ¹H NMR (500 MHz, Methanol-d₄) δ 9.64 (s, 1H), 8.84 (s, 1H), 8.19-8.06 (m, 2H), 8.00-7.92 (m, 1H), 7.77-7.68 (m, 2H), 7.52-7.37 (m, 2H), 7.23 (d, J=7.9 Hz, 1H), 3.63-3.44 (m, 2H), 3.03 (s, 3H), 2.61-2.50 (m, 1H), 2.50-2.39 (m, 1H).

Step 8. (S)-3-(3-(2-(7H-pyrrolo[2,3-d]pyrimidin-5-yl)thiazol-4-yl)phenyl)-3-hydroxy-1-methylpyrrolidin-2-one. The enantiomers of (R,S)-3-(3-(2-(7H-pyrrolo[2,3-d]pyrimidin-5-yl)thiazol-4-yl)phenyl)-3-hydroxy-1-methylpyrrolidin-2-one (90 mg) were separated by chiral 90 SFC using an SFC column, such as a Chiralpak ID, 5 μm, 250×21 mm column, Mobile phase: 35% methanol w/0.2% triethylamine, 65% CO₂) to yield the 1st eluting enantiomer, (S)-3-(3-(2-(7H-pyrrolo[2,3-d]pyrimidin-5-yl)thiazol-4-yl)phenyl)-3-hydroxy-1-methylpyrrolidin-2-one, SFC Rₜ=16.27 min, (21 mg) as a white solid.

### Example 53. (R)-3-(3-(2-(7H-Pyrrolo[2,3-d]pyrimidin-5-yl)thiazol-4-yl)phenyl)-3-hydroxy-1-methylpyrrolidin-2-one

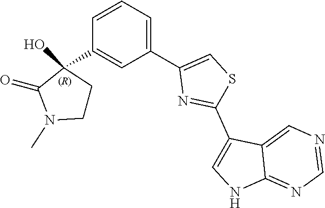

The chiral separation from Example 52 provided (R)-3-(3-(2-(7H-pyrrolo[2,3-d]pyrimidin-5-yl)thiazol-4-yl)phenyl)-3-hydroxy-1-methylpyrrolidin-2-one, SFC Rₜ=19.35 min, (7 mg) as a white film.

### Example 54. (R)-3-(3-(2-(5H-Pyrrolo[2,3-b]pyrazin-7-yl)thiazol-4-yl)phenyl)-3-hydroxy-1-methylpiperidin-2-one

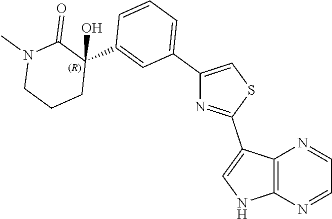

(R)-3-Hydroxy-1-methyl-3-(3-(2-(5-tosyl-5H-pyrrolo[2,3-b]pyrazin-7-yl)thiazol-4-yl)phenyl)piperidin-2-one (Intermediate 114, 130 mg, 0.232 mmol) was added to a mixture of NaOH (0.7 mL, 2.0 M in water, 1.4 mmol) and 1,4-dioxane (5 mL). The resultant mixture was heated to 60° C. for 0.5 h. After this time, the mixture was cooled to room temperature, poured into H₂O (10 mL), and extracted with methylene chloride/methanol, 10:1, 20 mL×3). The combined organic solvent extracts were washed with H₂O (10 mL×3) and concentrated to dryness under reduced pressure to afford the product, which was suspended in ethyl acetate (2 mL), diluted with petroleum ether (4 mL), and stirred at room temperature for 1 h. After this time, the mixture was filtered, and the filter cake was washed with petroleum ether (2 mL) and dried under reduced pressure to afford (R)-3-(3 (2-(5H-pyrrolo[2,3-b]pyrazin-7-yl)thiazol-4-yl)phenyl)-3-hydroxy-1-methylpiperidin-2-one (33.7 mg, 35%) as a yellow solid. LCMS(ESI): mass calcd. for C₂₁H₁₉N₅O₂S, 405.13; m/z found, 406.2 [M+H]⁺. ¹H NMR (400 MHz, DMSO-d₆) δ 12.79 (s, 1H), 8.64-8.62 (m, 2H), 8.43 (d, J=2.5 Hz, 1H), 8.08-8.05 (m, 2H), 7.95 (d, J=7.8 Hz, 1H), 7.44-7.39 (m, 1H), 7.27 (d, J=7.8 Hz, 1H), 5.72 (s, 1H), 3.48-3.41 (m, 2H), 2.99 (s, 3H), 2.17-2.10 (m, 1H), 2.07-2.00 (m, 1H), 1.93-1.83 (m, 1H), 1.65-1.52 (m, 1H).

### Example 55. (S)-3-(3-(2-(5H-Pyrrolo[2,3-b]pyrazin-7-yl)thiazol-4-yl)phenyl)-3-hydroxy-1-methylpiperidin-2-one

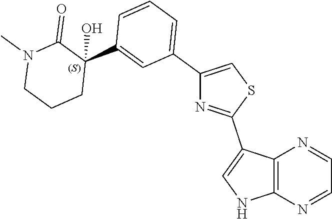

Sodium hydroxide (0.70 mL, 2.0 M in H₂O, 1.40 mmol) was added to a solution of (S)-3-hydroxy-1-methyl-3-(3-(2-(5-tosyl-5H-pyrrolo[2,3-b]pyrazin-7-yl)thiazol-4-yl)phenyl)piperidin-2-one (Intermediate 115, 130 mg, 0.232 mmol) and 1,4-dioxane (5 mL). The resultant mixture was heated to 60° C. for 1 h. After this time, the mixture was cooled to room temperature and concentrated to dryness under reduced pressure to afford the product, which was suspended in water (20 mL) and extracted with methylene chloride/MeOH (10:1, 30 mL×3). The combined organic solvent extracts were washed with water (30 mL×3), dried over anhydrous Na₂SO₄, filtered, and concentrated to dryness under reduced pressure. The product was then triturated with petroleum ether/ethyl acetate (3:1, 30 mL) and the suspension isolated via filtration. The filter cake was washed with petroleum ether/ethyl acetate (3:1, 20 mL) and dried under reduced pressure to afford (S)-3-(3-(2-(5H-pyrrolo[2,3-b]pyrazin-7-yl)thiazol-4-yl)phenyl)-3-hydroxy-1-methylpiperidin-2-one (34.6 mg, 36% yield) as a pale yellow solid. LCMS (ESI): mass calcd. for C₂₁H₁₉N₅O₂S, 405.13; m/z found, 406.1 [M+1]⁺. ¹H NMR (400 MHz, DMSO-d₆) δ 12.78 (br s, 1H), 8.62 (s, 1H), 8.61 (d, J=2.6 Hz, 1H), 8.42 (d, J=2.6 Hz, 1H), 8.06 (s, 1H), 8.04 (s, 1H), 7.94 (d, J=7.7 Hz, 1H), 7.43-7.38 (m, 1H), 7.26 (d, J=7.9 Hz, 1H), 5.71 (s, 1H), 3.50-3.40 (m, 2H), 2.98 (s, 3H), 2.16-2.00 (m, 2H), 1.93-1.81 (m, 1H), 1.65-1.52 (m, 1H).

### Example 56. (R)-3-(3-(4-(2-Fluoro-1H-pyrrolo[2,3-b]pyridin-3-yl)thiazol-2-yl)phenyl)-3-hydroxy-1-methylpyrrolidin-2-one

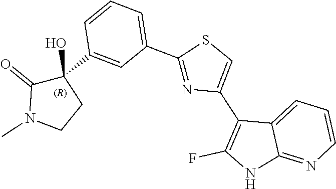

To a flask containing (R)-3-(3-(4-(2-fluoro-1-tosyl-1H-pyrrolo[2,3-b]pyridin-3-yl)thiazol-2-yl)phenyl)-3-hydroxy-1-methylpyrrolidin-2-one (Intermediate 99, 128 mg, 0.228 mmol) and 1,4-dioxane (2 mL) was added sodium hydroxide (0.68 mL, 2.0 M in water, 1.4 mmol). The resultant mixture was heated at 60° C. for 2 h. After this time, the reaction mixture was concentrated to dryness and purified by preparative HPLC using an HPLC column, such as a Boston Prime C18 150×30 mm×5 μm column (eluent: 30% to 60%, ACN and H₂O (with 0.05% NH₄OH and 10 mM NH₄HCO₃)). The pure fractions were collected and concentrated under vacuum. The residue was re-suspended in water (20 mL) and the resulting mixture was lyophilized to yield (R)-3-(3-(4-(2-fluoro-1H-pyrrolo[2,3-b]pyridin-3-yl)thiazol-2-yl)phenyl)-3-hydroxy-1-methylpyrrolidin-2-one (20 mg, 21%) as a yellow solid. LCMS(ESI): mass calcd. for C₂₁H₁₇FN₄O₂S, 408.11; m/z found, 409.1 [M+H]⁺. ¹H NMR (400 MHz, DMSO-d₆) δ 8.68-8.60 (m, 1H), 8.27-8.19 (m, 1H), 8.13-8.08 (m, 1H), 8.01-7.95 (m, 1H), 7.70 (s, 1H), 7.56-7.49 (m, 1H), 7.48-7.42 (m, 1H), 7.29-7.21 (m, 1H), 6.27 (s, 1H), 3.49-3.48 (m, 2H), 2.89 (s, 3H), 2.42-2.26 (m, 2H).

### Example 57. (R)-3-(3-(2-(2-Fluoro-1H-pyrrolo[2,3-b]pyridin-3-yl)thiazol-4-yl)phenyl)-3-hydroxy-1-methylpyrrolidin-2-one

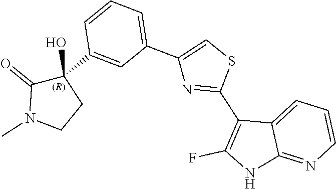

Sodium hydroxide (1.2 mL, 2 M in H₂O, 2.4 mmol) was added to a mixture of (R) 3-(3-(2-(2-fluoro-1-tosyl-1H-pyrrolo[2,3-b]pyridin-3-yl)thiazol-4-yl)phenyl)-3-hydroxy-1-methylpyrrolidin-2-one (Intermediate 101, 460 mg, 0.38 mmol) and 1,4-dioxane (2 mL). The resultant mixture was heated at 60° C. for 3 h. After this time, the mixture was cooled to room temperature, quenched with water (5 mL) and extracted with ethyl acetate (3×5 mL). The combined organic solvent extracts were dried over anhydrous Na₂SO₄, filtered, concentrated and purified by preparative HPLC using an HPLC column, such as a Phenomenex Gemini-NX C18 75×30 mm×3 μm column (eluent: 15% to 45%, CH₃CN/H₂O (0.05% NH₄OH)). The pure fractions were collected and concentrated under vacuum. The residue was re-suspended in water (5 mL) and the resulting mixture was lyophilized to yield (R)-3-(3-(2-(2-fluoro-1H-pyrrolo[2,3-b]pyridin-3-yl)thiazol-4-yl)phenyl)-3-hydroxy-1-methylpyrrolidin-2-one (20.7 mg, 13%) as a yellow solid. (ESI): mass calcd. for C₂₁H₁₇FN₄O₂S, 408.45; m/z found, 409.0 [M+1]⁺. ¹H NMR (400 MHz, DMSO-d₆) δ 8.83-8.74 (m, 1H), 8.28-8.20 (m, 1H), 8.09 (s, 1H), 8.01-7.95 (m, 2H), 7.51-7.42 (m, 1H), 7.38-7.31 (m, 2H), 6.14 (s, 1H), 3.51-3.46 (m, 1H), 3.43-3.40 (m, 1H), 2.90 (s, 3H), 2.43-2.35 (m, 1H), 2.34-2.25 (m, 1H).

### Example 58. (S)-4-(3-(4-(1H-Pyrrolo[2,3-b]pyridin-3-yl)thiazol-2-yl)phenyl)-5,6-dihydro-4H-cyclopenta[d]thiazol-4-ol

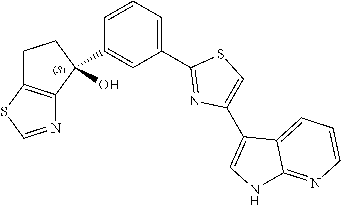

Sodium hydroxide (4 mL, 2.0 M in H₂O, 8 mmol) was added to a mixture of (S)-4-(3-(4-(1-(phenylsulfonyl)-1H-pyrrolo[2,3-b]pyridin-3-yl)thiazol-2-yl)phenyl)-5,6-dihydro-4H-cyclopenta[d]thiazol-4-ol (Intermediate 105, 700 mg, 1.3 mmol) and 1,4-dioxane (20 mL). The resultant mixture was heated at 60° C. for 3 h. After this time, the mixture was cooled to room temperature, concentrated to dryness and purified by preparative HPLC using an HPLC column, such as an Xtimate C18 150×40 mm×5 μm column (eluent: 35% to 65%, CH₃CN and H₂O (with 10 mM NH₄HCO₃)). The product was suspended in water (10 mL) and the mixture was frozen by inserting it into a −78° C. bath, and then lyophilized to yield (S)-4-(3-(4-(1H-pyrrolo[2,3-b]pyridin-3-yl)thiazol-2-yl)phenyl)-5,6-dihydro-4H-cyclopenta[d]thiazol-4-ol (83.1 mg, 16%) as a colorless solid. LCMS(ESI): mass calcd. for C₂₂H₁₆N₄OS₂, 416.08; m/z found, 417.1 [M+H]⁺. ¹H NMR (400 MHz, DMSO-d₆) δ 11.98 (br s, 1H), 9.03 (s, 1H), 8.61-8.53 (m, 1H), 8.36-8.27 (m, 1H), 8.15-8.05 (m, 2H), 7.97-7.88 (m, 2H), 7.52-7.45 (m, 1H), 7.43-7.35 (m, 1H), 7.25-7.19 (m, 1H), 6.08 (br s, 1H), 3.19-3.07 (m, 1H), 3.04-2.93 (m, 1H), 2.87-2.80 (m, 2H).

### Example 59. (R)-4-(3-(4-(1H-Pyrrolo[2,3-b]pyridin-3-yl)thiazol-2-yl)phenyl)-5,6-dihydro-4H-cyclopenta[d]thiazol-4-ol

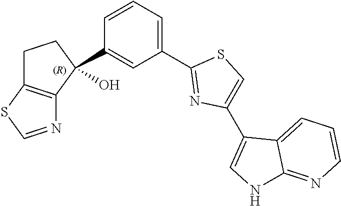

Sodium hydroxide (3.85 mL, 2.0 M in H₂O, 7.70 mmol) was added to a mixture of (R)-4-(3-(4-(1-(phenylsulfonyl)-1H-pyrrolo[2,3-b]pyridin-3-yl)thiazol-2-yl)phenyl)-5,6-dihydro-4H-cyclopenta[d]thiazol-4-ol (Intermediate 107, 700 mg, 1.26 mmol), and 1,4-dioxane (20 mL). The resultant mixture was heated at 60° C. for 3 h. After this time, the mixture was cooled to room temperature, concentrated under reduced pressure, and purified by preparative HPLC using an HPLC column, such as an Xtimate C18 150×40 mm×5 μm column (eluent: 35% to 65%, CH₃CN and H₂O (0.05% NH₄OH+10 mM NH₄HCO₃)). The product was suspended in water (5 mL), the mixture was frozen by inserting it into a −78° C. bath, and then lyophilized to yield (R)-4-(3-(4-(1H-pyrrolo[2,3-b]pyridin-3-yl)thiazol-2-yl)phenyl)-5,6-dihydro-4H-cyclopenta[d]thiazol-4-ol (62.3 mg, 12%) as a colorless solid. LCMS(ESI): mass calcd. for C₂₂H₁₆N₄OS₂, 416.08; m/z found, 417.0 [M+H]⁺. ¹H NMR (400 MHz, DMSO-d₆) δ 11.97 (br s, 1H), 9.03 (s, 1H), 8.60-8.52 (m, 1H), 8.34-8.26 (m, 1H), 8.15-8.03 (m, 2H), 7.97-7.87 (m, 2H), 7.53-7.45 (m, 1H), 7.44-7.35 (m, 1H), 7.26-7.18 (m, 1H), 6.08 (br s, 1H), 3.19-3.07 (m, 1H), 3.05-2.94 (m, 1H), 2.88-2.80 (m, 2H).

### Example 60. (R)-1-(3-(2-(1H-Pyrrolo[2,3-b]pyridin-3-yl)thiazol-4-yl)phenyl)-1-(pyridin-2-yl)ethan-1-ol

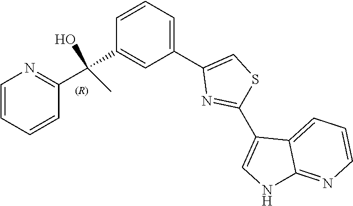

Step A. (R,S)-1-(3-(2-(1H-pyrrolo[2,3-b]pyridin-3-yl)thiazol-4-yl)phenyl)-1-(pyridin-2-yl)ethan-1-ol. Sodium hydroxide (3.0 mL, 6.0 mmol, 2.0 M in H₂O) was added to a mixture of 1-(3-(2-(1-(phenylsulfonyl)-1H-pyrrolo[2,3-b]pyridin-3-yl)thiazol-4-yl)phenyl)-1-(pyridin-2-yl)ethanol (Intermediate 110, 2.0 g, 3.7 mmol) and 1,4-dioxane (10 mL). The resultant mixture was heated at 60° C. for 2 h. After this time, the mixture was cooled to room temperature, concentrated under reduced pressure, and purified by preparative HPLC using an HPLC column, such as a YMC-Triart Prep C18 column (eluent: 48% to 58%, CH₃CN and H₂O (with 0.04% NH₄OH+10 mM NH₄HCO₃)). The product was suspended in water (10 mL), the mixture was frozen by inserting it into a −78° C. bath and then lyophilized to yield (R,S)-1-(3-(2-(1H-pyrrolo[2,3-b]pyridin-3-yl)thiazol-4-yl)phenyl)-1-(pyridin-2-yl)ethan-1-ol (360 mg, 24%) as a colorless solid. LCMS (ESI): mass calcd. for C₂₃H₁₈N₄OS, 398.12; m/z found, 399.1 [M+H]⁺.

Step B. (R)-1-(3-(2-(1H-pyrrolo[2,3-b]pyridin-3-yl)thiazol-4-yl)phenyl)-1-(pyridin-2-yl)ethan-1-ol. The enantiomers of (R,S)-1-(3-(2-(1H-pyrrolo[2,3-b]pyridin-3-yl)thiazol-4-yl)phenyl)-1-(pyridin-2-yl)ethanol (360 mg, 0.903 mmol) were separated by preparative SFC using an SFC column, such as a DAICEL CHIRALPAK AD 250 mm×30 mm×10 μm column (isocratic elution: 40%: 60% supercritical CO₂: i-PrOH (containing 0.1% of 25% aqueous NH₃)). The first eluting enantiomer was (R)-1-(3-(2-(1H-pyrrolo[2,3-b]pyridin-3-yl)thiazol-4-yl)phenyl)-1-(pyridin-2-yl)ethan-1-ol and the second eluting enantiomer was (S)-1-(3-(2-(1H-pyrrolo[2,3-b]pyridin-3-yl)thiazol-4-yl)phenyl)-1-(pyridin-2-yl)ethan-1-ol (Example 61). (R)-1-(3-(2-(1H-pyrrolo[2,3-b]pyridin-3-yl)thiazol-4-yl)phenyl)-1-(pyridin-2-yl)ethan-1-ol was further purified by preparative HPLC using an HPLC column, such as a Boston Prime C18 150×30 mm×5 μm column (eluent: 50% to 80%, CH₃CN and H₂O (with 0.04% NH₄OH+10 mM NH₄HCO₃)). This product was suspended in water (10 mL), the mixture was frozen by inserting it into a −78° C. bath and then lyophilized to yield (R)-1-(3-(2-(1H-pyrrolo[2,3-b]pyridin-3-yl)thiazol-4-yl)phenyl)-1-(pyridin-2-yl)ethan-1-ol (81.7 mg, 23%) as a colorless solid. LCMS (ESI): mass calcd. for C₂₃H₁₈N₄OS, 398.12; m/z found, 399.0 [M+H]⁺. ¹H NMR (400 MHz, DMSO-d₆) δ 12.30 (br s, 1H), 8.61-8.57 (m, 1H), 8.55 (d, J=4.6 Hz, 1H), 8.39-8.36 (m, 1H), 8.27 (d, J=2.7 Hz, 1H), 8.17 (s, 1H), 7.90 (s, 1H), 7.86 (d, J=7.7 Hz, 1H), 7.82-7.77 (m, 1H), 7.76-7.73 (m, 1H), 7.54-7.49 (m, 1H), 7.42-7.36 (m, 1H), 7.36-7.31 (m, 1H), 7.29-7.24 (m, 1H), 6.06 (s, 1H), 1.96 (s, 3H). SFC Rₜ=1.645 min.

### Example 61. (S)-1-(3-(2-(1H-Pyrrolo[2,3-b]pyridin-3-yl)thiazol-4-yl)phenyl)-1-(pyridin-2-yl)ethan-1-ol

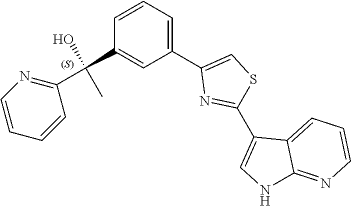

The chiral separation from Example 60, Step B, yielded (S)-1-(3-(2-(1H-pyrrolo[2,3-b]pyridin-3-yl)thiazol-4-yl)phenyl)-1-(pyridin-2-yl)ethan-1-ol (84.7 mg, 24%) as a yellow solid. LCMS (ESI): mass calcd. for C₂₃H₁₈N₄OS, 398.12; m/z found, 399.0 [M+H]⁺. ¹H NMR (400 MHz, DMSO-d₆) δ 8.62-8.57 (m, 1H), 8.56-8.53 (m, 1H), 8.40-8.34 (m, 1H), 8.27 (s, 1H), 8.16 (s, 1H), 7.90 (s, 1H), 7.88-7.84 (m, 1H), 7.82-7.77 (m, 1H), 7.76-7.72 (m, 1H), 7.54-7.46 (m, 1H), 7.43-7.36 (m, 1H), 7.36-7.30 (m, 1H), 7.30-7.23 (m, 1H), 6.32-5.85 (m, 1H), 1.96 (s, 3H). SFC Rₜ=2.267 min.

### Example 62. (R)-7-(3-(2-(5H-Pyrrolo[2,3-b]pyrazin-7-yl-6-d)thiazol-4-yl)phenyl)-6,7-dihydro-5H-pyrrolo[1,2-a]imidazol-7-ol

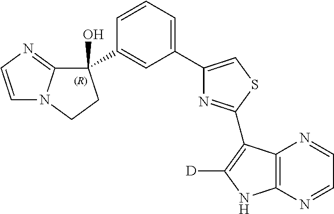

(R)-7-(3-(2-(5H-pyrrolo[2,3-b]pyrazin-7-yl-6-d)thiazol-4-yl)phenyl)-6,7-dihydro-5H-pyrrolo[1,2-a]imidazol-7-ol was prepared in a manner analogous to Example 18 using (R)-7-(3-(2-(5-tosyl-5H-pyrrolo[2,3-b]pyrazin-7-yl-6-d)thiazol-4-yl)phenyl)-6,7-dihydro-5H-pyrrolo[1,2-a]imidazol-7-ol (Intermediate 112) instead of (S)-7-(3-(2-(5-tosyl-5H-pyrrolo[2,3-b]pyrazin-7-yl-6-d)thiazol-4-yl)phenyl)-6,7-dihydro-5H-pyrrolo[1,2-a]imidazol-7-ol (Intermediate 42). LCMS (ESI): mass calcd. for C₂₁H₁₅DN₆OS, 401.12; m/z found 402.0 [M+H]⁺. SFC Rₜ=3.73 min. ¹H NMR (400 MHz, DMSO-d₆): δ 8.62 (d, J=2.4 Hz, 1H), 8.42 (d, J=2.4 Hz, 1H), 8.20 (s, 1H), 8.02 (s, 1H), 7.99-7.91 (m, 1H), 7.50-7.36 (m, 2H), 7.20 (d, J=1.1 Hz, 1H), 7.03 (d, J=1.1 Hz, 1H), 6.19 (s, 1H), 4.21-4.06 (m, 2H), 2.97-2.79 (m, 2H)

### Example 63. (R)-7-(3-(2-(5H-Pyrrolo[2,3-b]pyrazin-7-yl-2-d)thiazol-4-yl)phenyl)-6,7-dihydro-5H-pyrrolo[1,2-a] imidazol-7-ol

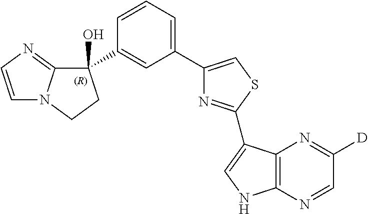

(R)-7-(3-(2-(5H-pyrrolo[2,3-b]pyrazin-7-yl-2-d)thiazol-4-yl)phenyl)-6,7-dihydro-5H-pyrrolo[1,2-a]imidazol-7-ol was prepared in a manner analogous to Example 19 using (R)-7-(3-(2-(5-tosyl-5H-pyrrolo[2,3-b]pyrazin-7-yl-2-d)thiazol-4-yl)phenyl)-6,7-dihydro-5H-pyrrolo[1,2-a]imidazol-7-ol (Intermediate 117) instead of (S)-7-(3-(2-(5-tosyl-5H-pyrrolo[2,3-b]pyrazin-7-yl-2-d)thiazol-4-yl)phenyl)-6,7-dihydro-5H-pyrrolo[1,2-a]imidazol-7-ol (Intermediate 45). LCMS (ESI): mass calcd. For C₂₁H₁₅DN₆OS, 401.12; m/z found, 402.1 [M+H]. SFC Rₜ=1.81 min.

### Example 64. (S)-1-(3-(2-(1H-Pyrrolo[2,3-b]pyridin-3-yl)thiazol-4-yl)phenyl)-1-(thiazol-2-yl)ethan-1-ol

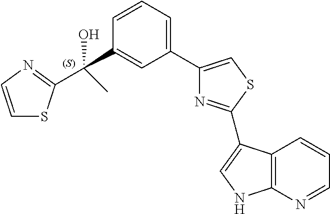

The (R) and (S) enantiomers of 1-(3-(2-(1H-Pyrrolo[2,3-b]pyridin-3-yl)thiazol-4-yl)phenyl)-1-(thiazol-2-yl)ethanol (Intermediate 120, Step D, 75.0 mg, 0.185 mmol) were separated by SFC over an AD 250 mm×30 mm×10 μm column to give the two enantiomers. The first eluting enantiomer, the title compound, (5)-1-(3-(2-(1H-pyrrolo[2,3-b]pyridin-3-yl)thiazol-4-yl)phenyl)-1-(thiazol-2-yl)ethan-1.01 (21 mg, 27%) was obtained as a white solid. LC-MS (ESI): mass calcd. for C₂₁H₁₆N₄OS₂ 404.08 m/z, found 405.1 [M+H]⁺. ¹H NMR (400 MHz, DMSO-d₆) δ 12.30 (br s, 1H), 8.64 (d, J=8.0 Hz, 1H), 8.39-8.36 (m, 1H), 8.30-8.25 (m, 2H), 7.93 (s, 1H), 7.91 (s, 1H), 7.77 (d, J=3.2 Hz, 1H), 7.64 (d, J=3.2 Hz, 1H), 7.57 (d, J=8.0 Hz, 1H), 7.47-7.41 (m, 1H), 7.36-7.31 (m, 1H), 6.82-6.77 (m, 1H), 2.00 (s, 3H).

### Example 65. (R)-1-(3-(2-(1H-Pyrrolo[2,3-b]pyridin-3-yl)thiazol-4-yl)phenyl)-1-(thiazol-2-yl)ethan-1-ol

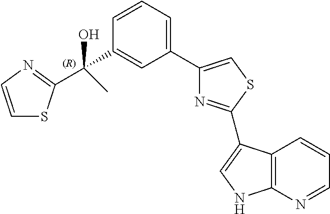

The chiral separation described in Example 64 provided the second eluting enantiomer, (R)-1-(3-(2-(1H-pyrrolo[2,3-b]pyridin-3-yl)thiazol-4-yl)phenyl)-1-(thiazol-2-yl)ethan-1-ol (20 mg, 26%) as a white solid. LC-MS (ESI): mass calcd. for C₂₁H₁₆N₄OS₂ 404.08 m/z, found 405.1 [M+H]⁺. ¹H NMR (400 MHz, DMSO-d₆) δ 12.30 (br s, 1H), 8.64 (d, J=8.0 Hz, 1H), 8.39-8.36 (m, 1H), 8.30-8.25 (m, 2H), 7.93 (s, 1H), 7.91 (s, 1H), 7.77 (d, J=3.2 Hz, 1H), 7.64 (d, J=3.2 Hz, 1H), 7.57 (d, J=8.0 Hz, 1H), 7.47-7.41 (m, 1H), 7.36-7.31 (m, 1H), 6.82-6.77 (m, 1H), 2.00 (s, 3H).

### Example 66. (R)-3-Hydroxy-1-methyl-3-(3-(5-methyl-2-(5H-pyrrolo[2,3-b]pyrazin-7-yl)thiazol-4-yl)phenyl)pyrrolidin-2-one

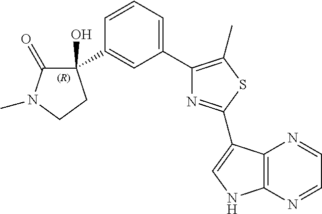

Sodium hydroxide (0.22 mL, 2.0 M in water, 0.44 mmol) was added to a solution of (R)-3-hydroxy-1-methyl-3-(3-(5-methyl-2-(5-tosyl-5H-pyrrolo[2,3-b]pyrazin-7-yl)thiazol-4-yl)phenyl)pyrrolidin-2-one (Intermediate 121, 35 mg, 0.063 mmol) and 1,4-dioxane (2 mL). The resultant mixture was heated at 60° C. for 0.5 hours. After this time, the mixture was cooled to room temperature, diluted with H₂O (20 mL), and extracted with ethyl acetate (20 mL×4). The combined organic extracts were washed with brine (50 mL), dried over anhydrous Na₂SO₄, filtered, and concentrated to dryness under reduced pressure to afford the product, which was purified by prep. HPLC using an Xtimate C18 250×50 mm×10 μm column (eluent: 35% to 65% (v/v) CH₃CN and H₂O with (0.04% NH₃H₂O+10 mM NH₄HCO₃) to afford the title compound (10.3 mg, 40%) as a colorless solid. LCMS (ESI): mass calcd. for C₂₁H₁₉N₅O₂S 405.13 m/z, found 406.2 [M+H]⁺. ¹H NMR (400 MHz, DMSO-d₆) δ13.32-11.62 (m, 1H), 8.58 (d, J=2.7 Hz, 1H), 8.53 (s, 1H), 8.39 (d, J=2.4 Hz, 1H), 7.78-7.73 (m, 1H), 7.67-7.62 (m, 1H), 7.46 (m, 1H), 7.39-7.33 (m, 1H), 6.09 (br s, 1H), 3.50-3.45 (m, 1H), 3.38-3.37 (m, 1H), 2.86 (s, 3H), 2.60 (s, 3H), 2.41-2.23 (m, 2H).

### Example 67. (R)-3-(3-(2-(5H-Pyrrolo[2,3-b]pyrazin-7-yl)thiazol-4-yl)-4-methylphenyl)-3-hydroxy-1-methylpyrrolidin-2-one

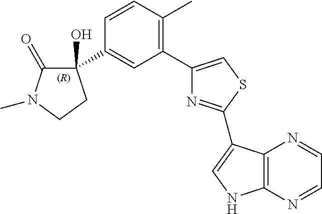

(R)-3-Hydroxy-1-methyl-3-(4-methyl-3-(2-(5-tosyl-5H-pyrrolo[2,3-b]pyrazin-7-yl)thiazol-4-yl)phenyl)pyrrolidin-2-one (Intermediate 130, 190 mg, 0.339 mmol) was added to a solution of NaOH (1.02 mL, 2 M in water, 2.04 mmol) and 1,4-dioxane (5 mL). The reaction mixture was heated at 60° C. for 1 hour. After this time, the mixture was cooled to room temperature, poured into H₂O (10 mL), and extracted with ethyl acetate:methanol (10:1, 20 mL×3). The combined organic extracts were washed with H₂O (10 mL×3) and concentrated to dryness under reduced pressure to afford a residue, which was purified by preparative HPLC using a Boston Green ODS 150 mm×30 mm, 5 μm (eluent: 30% to 60% (v/v) water (0.04% NH₃H₂O+10 mM NH₄HCO₃)—CH₃CN) to afford pure product. The product was then suspended in water (5 mL), the mixture was frozen using dry ice, and then lyophilized to dryness to afford the title compound (64.8 mg, 47%) as a white solid. LC-MS (ESI): mass calcd. For C₂₁H₁₉NSO₂S 405.13 m/z found 406.2 [M+H]⁺. ¹H NMR (400 MHz, DMSO-d₆) δ 8.61 (d, J=2.4 Hz, 1H), 8.56 (s, 1H), 8.41 (d, J=2.4 Hz, 1H), 8.45-8.36 (m, 1H), 7.75-7.61 (m, 2H), 7.32-7.19 (m, 2H), 6.00 (s, 1H), 3.49-3.38 (m, 2H), 2.83 (s, 3H), 2.70-2.63 (m, 1H), 2.67 (s, 1H), 2.44 (s, 3H), 2.38-2.33 (m, 1H), 2.27-2.20 (m, 1H).

### Example 68. (R)-3-(3-(2-(5H-Pyrrolo[2,3-b]pyrazin-7-yl)thiazol-4-yl)-5-methylphenyl)-3-hydroxy-1-methylpyrrolidin-2-one

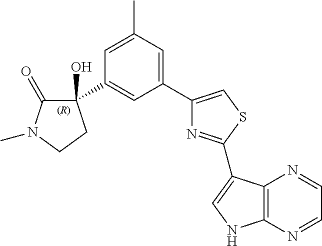

(R)-3-Hydroxy-1-methyl-3-(3-methyl-5-(2-(5-tosyl-5H-pyrrolo[2,3-b]pyrazin-7-yl)thiazol-4-yl)phenyl)pyrrolidin-2-one (Intermediate 133, 160 mg, 0.286 mmol) was added to a mixture of NaOH (0.8 mL, 2 M in water, 1.6 mmol) and 1,4-dioxane (8 mL). The reaction mixture was heated at 60° C. for 1 hour and then allowed to cool to room temperature. The reaction mixture was then diluted with H₂O (10 mL) and extracted with dichloromethane:methanol (10:1, 20 mL×3). The combined organic extracts were washed with H₂O (10 mL×3) and concentrated to dryness under reduced pressure. The resulting residue was triturated with acetonitrile (10 mL) and filtered. The filtrate cake was dried under reduced pressure to afford the title compound (35.7 mg, 31%) as a white solid. LC-MS (ESI): mass calcd. For C₂₁H₁₉N₅O₂S 405.13 m/z found 406.2 [M+H]⁺. ¹H NMR (400 MHz, MeOD-d₄) δ 8.56 (d, J=2.4 Hz, 1H), 8.54 (s, 1H), 8.35 (d, J=2.6 Hz, 1H), 7.88 (s, 1H), 7.77 (s, 2H), 7.25 (s, 1H), 3.62-3.46 (m, 2H), 3.00 (s, 3H), 2.63-2.52 (m, 1H), 2.45-2.34 (m, 4H).

### Example 69. (R)-3-(5-(2-(5H-Pyrrolo[2,3-b]pyrazin-7-yl)thiazol-4-yl)-2-methylphenyl)-3-hydroxy-1-methylpyrrolidin-2-one

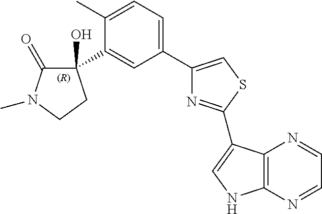

To a microwave vial containing 4-bromo-2-(5H-pyrrolo[2,3-b]pyrazin-7-yl)thiazole (Intermediate 149, 59 mg, 0.21 mmol), (R)-3-hydroxy-1-methyl-3-(2-methyl-5-(4,4,5,5-tetramethyl-1,3,2-dioxaborolan-2-yl)phenyl)pyrrolidin-2-one (Intermediate 135, 70 mg, 0.21 mmol) and K₃PO₄ (135 mg, 0.636 mmol) was added 1,4-dioxane (2 mL), and H₂O (0.4 mL). The mixture was sparged with Ar for 5 minutes and then treated with Pd(dtbpf)Cl₂ (28 mg, 0.04 mmol). The mixture was sparged with Ar for another 5 minutes and the resultant mixture was heated at 80° C. via microwave irradiation for 1 hour. The mixture was cooled to room temperature and quenched with H₂O (50 mL) and extracted with dichloromethane:methanol (10:1, 30 mL×3). The combined organic extracts were dried over anhydrous Na₂SO₄, filtered and concentrated under reduced pressure to afford the product, which was further purified by preparative HPLC using a Xtimate C18 250×50 mm×10 μm column (eluent: 30% to 60% (v/v) CH₃CN and H₂O with 0.04% NH₃H₂O+10 mM NH₄HCO₃) to afford the title compound (10.8 mg, 13%) as a white solid. LCMS (ESI): mass calcd. for C₂₁H₁₉N₅O₂S 405.13 m/z, found 406.2 [M+H]⁺. ¹H NMR (400 MHz, DMSO-d₆) δ 12.75 (br s, 1H), 8.61 (d, J=2.4 Hz, 1H), 8.58 (s, 1H), 8.42 (d, J=2.4 Hz, 1H), 8.12 (s, 1H), 7.89 (s, 1H), 7.85 (d, J=7.6 Hz, 1H), 7.24 (d, J=7.8 Hz, 1H), 6.05 (s, 1H), 3.54-3.48 (m, 1H), 3.47-3.41 (m, 1H), 2.90 (s, 3H), 2.35-2.31 (m, 1H), 2.28 (s, 3H), 2.22-2.13 (m, 1H).

### Example 70. (S)-3-(5-(2-(5H-Pyrrolo[2,3-b]pyrazin-7-yl)thiazol-4-yl)-2-methylphenyl)-3-hydroxy-1-methylpyrrolidin-2-one

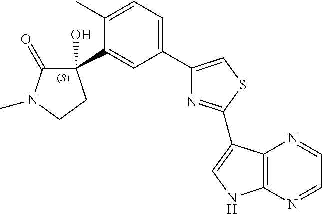

4-Bromo-2-(5H-pyrrolo[2,3-b]pyrazin-7-yl)thiazole (Intermediate 149, 85.0 mg, 0.302 mmol), (S)-3-hydroxy-1-methyl-3-(2-methyl-5-(4,4,5,5-tetramethyl-1,3,2-dioxaborolan-2-yl)phenyl)pyrrolidin-2-one (Intermediate 134, 100 mg, 0.302 mmol), K₃PO₄ (197 mg, 0.928 mmol), 1,4-dioxane (8 mL), and H₂O (2 mL) were added to a microwave tube. The mixture was sparged with Ar for 5 minutes and then treated with Pd(dtbpf)Cl₂ (20 mg, 0.031 mmol). The mixture was sparged with Ar for another 5 minutes and then heated at 80° C. via microwave irradiation for 1 hour. After this time, the mixture was cooled to room temperature, diluted with a solution of 1,4-dioxane:methanol (10:1, 50 mL), filtered, and concentrated to dryness under reduced pressure to afford the product, which was purified by preparative HPLC using a Xtimate C18 250 mm×50 mm×10 μm (eluent: 30% to 60% (v/v) CH₃CN and H₂O with 0.04% NH₃H₂O+10 mM NH₄HCO₃ to afford the title compound (11.0 mg, 9%) as a yellow solid. LC-MS (ESI): mass calcd. For C₂₁H₁₉N₅O₂S 405.13 m/z found 406.0 [M+H]⁺. ¹H NMR (400 MHz, DMSO-d₆) δ 12.76 (br s, 1H), 8.62 (d, J=2.8 Hz, 1H), 8.59 (s, 1H), 8.42 (d, J=2.8 Hz, 1H), 8.13 (d, J=2.0 Hz, 1H), 7.90 (s, 1H), 7.86 (dd, J=1.8, 7.8 Hz, 1H), 7.25 (d, J=7.8 Hz, 1H), 6.07 (s, 1H), 3.55-3.43 (m, 2H), 2.91 (s, 3H), 2.37-2.33 (m, 1H), 2.29 (s, 3H), 2.22-2.15 (m, 1H).

### Example 71. (R)-3-Hydroxy-1-methyl-3-(3-(2-(2-methyl-1H-pyrrolo[2,3-b]pyridin-3-yl)thiazol-4-yl)phenyl)pyrrolidin-2-one

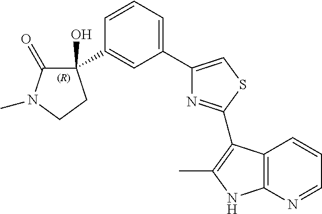

(R)-3-Hydroxy-1-methyl-3-(3-(2-(2-methyl-1-tosyl-1H-pyrrolo[2,3-b]pyridin-3-yl)thiazol-4-yl)phenyl)pyrrolidin-2-one (Intermediate 136, 120 mg, 0.215 mmol) was added to a mixture of NaOH (0.6 mL, 2 M in water, 1.2 mmol) and 1,4-dioxane (4 mL). The reaction mixture was heated at 70° C. for 6 hours. After this time, the reaction mixture was cooled to room temperature and concentrated under reduced pressure to afford the product, which was further purified by preparative HPLC using a Boston Prime C18 150×30 mm×5 μm column (eluent: 30% to 60% (v/v) CH₃CN and H₂O with 0.05% NH₃) to afford pure product. The product was suspended in water (10 mL) and frozen using dry ice/acetone, and then lyophilized to dryness to afford the title compound (12 mg, 13%) as a white solid. LC-MS (ESI): mass calcd. For C₂₂H₂₀N₄O₂S 404.13 m/z found 405.1 [M+H]⁺. ¹H NMR (400 MHz, MeOD-d₄) δ 8.67 (d, J=7.6 Hz, 1H), 8.22 (s, 1H), 8.15 (s, 1H), 7.98 (d, J=7.3 Hz, 1H), 7.75 (s, 1H), 7.52-7.39 (m, 2H), 7.28-7.20 (m, 1H), 3.64-3.41 (m, 2H), 3.01 (s, 3H), 2.83 (s, 3H), 2.59-2.37 (m, 2H).

### Example 72. (R)-3-(4-(3-(3-Hydroxy-1-methyl-2-oxopyrrolidin-3-yl)phenyl)thiazol-2-yl)-1H-pyrrolo[2,3-b]pyridine-5-carbonitrile

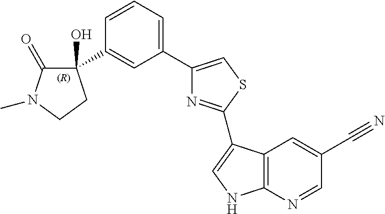

To a flask containing (R)-3-(4-(3-(3-Hydroxy-1-methyl-2-oxopyrrolidin-3-yl)phenyl)thiazol-2-yl)-1-tosyl-1H-pyrrolo[2,3-b]pyridine-5-carbonitrile (Intermediate 137, 90 mg) was added NaOH (0.5 mL, 2 M in water, 1.0 mmol) and 1,4-dioxane (5 mL). The reaction mixture was heated at 60° C. for 1 hour and then cooled to room temp, diluted with H₂O (10 mL), and extracted with dichloromethane:methanol (10:1, 20 mL×3). The combined organic extracts were washed with H₂O (10 mL×3). The suspension was concentrated to dryness under reduced pressure to afford the product, which was purified by preparative HPLC using a Boston Green ODS 150×30 mm, 5 μm column (eluent: 25% to 55% (v/v) CH₃CN and water (0.225% FA)) to afford pure product. The product was suspended in water (5 mL) and frozen using dry ice, and then lyophilized to dryness to afford the title compound (34.6 mg, 53%) as a white solid. LC-MS (ESI): mass calcd. For C₂₂H₁₇N₅O₂S 415.11 m/z found 416.1 [M+H]⁺. ¹H NMR (400 MHz, MeOD-d₄) S 9.10 (d, J=2.0 Hz, 1H), 8.66 (d, J=2.0 Hz, 1H), 8.27 (s, 1H), 8.16 (s, 1H), 8.01-7.95 (m, 1H), 7.77 (s, 1H), 7.53-7.46 (m, 2H), 3.68-3.52 (m, 2H), 3.05 (s, 3H), 2.63-2.39 (m, 2H).

### Example 73. (R)-3-Hydroxy-1-methyl-3-(3-(2-(6-methyl-1H-pyrrolo[2,3-b]pyridin-3-yl)thiazol-4-yl)phenyl)pyrrolidin-2-one

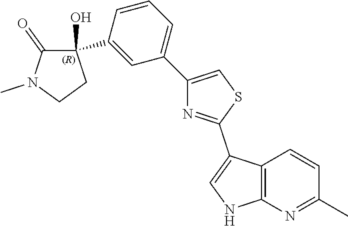

(R)-3-Hydroxy-1-methyl-3-(3-(2-(6-methyl-1-tosyl-1H-pyrrolo[2,3-b]pyridin-3-yl)thiazol-4-yl)phenyl)pyrrolidin-2-one (Intermediate 138, 120 mg, 0.22 mmol) was added to a mixture of NaOH (0.64 mL, 2 M in water, 1.3 mmol) and 1,4-dioxane (6 mL). The reaction mixture was heated at 60° C. for 1 hour. The mixture was cooled to room temperature, diluted with H₂O (10 mL) and extracted with dichloromethane:methanol (10:1, 20 mL×3). The combined organic extracts were washed with H₂O (10 mL×3). The suspension was concentrated to dryness under reduced pressure to afford the product, which was purified by preparative HPLC using a Boston Green ODS 150 mm×30 mm×5 μm column (eluent: 25% to 55% (v/v) CH₃CN and water (0.225% FA)) to afford pure product. The product was suspended in water (5 mL), the mixture frozen using dry ice, and then lyophilized to dryness to afford the title compound (42.3 mg, 47%) as a gray solid. LC-MS (ESI): mass calcd. For C₂₂H₂₀N₄O₂S 404.13 m/z found 405.1 [M+H]⁺. ¹H NMR (400 MHz, MeOD-d₄) δ 8.61 (d, J=8.2 Hz, 1H), 8.14 (s, 1H), 8.03-7.90 (m, 2H), 7.68 (s, 1H), 7.49-7.40 (m, 2H), 7.19 (d, J=8.2 Hz, 1H), 3.61-3.48 (m, 2H), 3.02 (s, 3H), 2.63 (s, 3H), 2.58-2.40 (m, 2H).

### Example 74. (R)-3-(3-(2-(2,5-Dimethyl-1H-pyrrolo[2,3-b]pyridin-3-yl)thiazol-4-yl)phenyl)-3-hydroxy-1-methylpyrrolidin-2-one

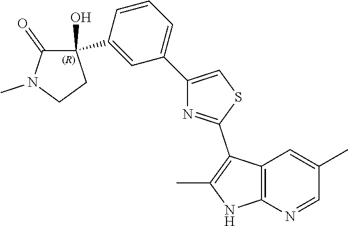

To a flask containing (R)-3-(3-(2-(2,5-dimethyl-1-tosyl-1H-pyrrolo[2,3-b]pyridin-3-yl)thiazol-4-yl)phenyl)-3-hydroxy-1-methylpyrrolidin-2-one (Intermediate 183, 120 mg, 0.21 mmol) and 1,4-dioxane (6 mL) was added NaOH (0.42 mL, 3.0 M in water, 1.3 mmol). The resultant mixture was heated at 70° C. for 2 hours. The mixture was cooled to room temperature and concentrated to dryness under reduced pressure to give the product, which was further purified by preparative HPLC using a YMC-Triart Prep C18 250×50 mm×10 μm column (eluent: 36% to 66% (v/v) CH₃CN and H₂O with 0.04% NH₃H₂O+10 mM NH₄HCO₃) to afford pure product. The product was suspended in water (10 mL), the mixture frozen using dry ice/acetone, and then lyophilized to dryness to afford the title compound (42.3 mg, 48%) as a white solid. LCMS (ESI): mass calcd. for C₂₃H₂₂N₄O₂S 418.15 m/z, found 419.2 [M+H]⁺. ¹H NMR (400 MHz, DMSO-d₆) δ 8.39 (s, 1H), 8.15-8.08 (m, 2H), 8.00 (s, 1H), 7.95 (d, J=7.6 Hz, 1H), 7.49-7.42 (m, 1H), 7.40-7.35 (m, 1H), 6.17 (br s, 1H), 3.52-3.46 (m, 1H), 3.42-3.40 (m, 1H), 2.87 (s, 3H), 2.77 (s, 3H), 2.47 (s, 3H), 2.43-2.36 (m, 1H), 2.34-2.27 (m, 1H).

### Example 75. (R)-7-(3-(2-(1H-Pyrazolo[3,4-b]pyridin-3-yl)thiazol-4-yl)phenyl)-6,7-dihydro-5H-pyrrolo[1,2-a]imidazol-7-ol

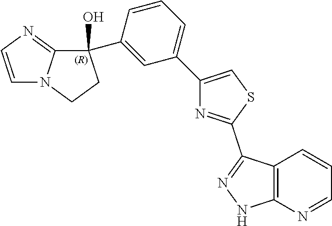

To a microwave vial containing 4-bromo-2-(1H-pyrazolo[3,4-b]pyridin-3-yl)thiazole (250 mg, 0.889 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13. 348 mg, 1.07 mmol) and K₃PO₄ (363 mg, 2.67 mmol) was added 1,4-dioxane (8 mL), and H₂O (2 mL). The mixture was sparged with Ar for 5 minutes and then treated with Pd(dtbpf)Cl₂ (58 mg, 0.089 mmol). The mixture was sparged with Ar for another 5 minutes and then heated at 90° C. via microwave irridiation for 1 hour. Since starting material still remained, additional Pd(dtbpf)Cl₂ (58 mg, 0.089 mmol) was added to above mixture and the resultant mixture heated at 110° C. via microwave irradiation for 2 hours. After this time, the mixture was cooled to room temperature and concentrated under reduced pressure to afford the product, which was initially purified by FCC (eluent: petroleum ether:ethyl acetate=1:0 to 0:1), and then purified by preparative HPLC using a Xtimate C18 150×40 mm×10 μm column (eluent: 22% to 52% (v/v) CH₃CN and H₂O with 0.04% NH₃H₂O+10 mM NH₄HCO₃) to afford pure product. The product was suspended in water (10 mL), the mixture frozen using dry ice/acetone, and then lyophilized to dryness to afford the title compound (50.7 mg, 14%) as a white solid. LC-MS (ESI): mass calcd. For C₂₁H₁₆N₆O S 400.11 m/z found 401.1 [M+H]⁺. ¹H NMR (400 MHz, DMSO-d₆) δ 14.19 (s, 1H), 8.80 (d, J=7.8 Hz, 1H), 8.67 (d, J=3.5 Hz, 1H), 8.27 (s, 1H), 8.15 (s, 1H), 8.04-7.97 (m, 1H), 7.55-7.41 (m, 3H), 7.24 (s, 1H), 7.12 (s, 1H), 6.23 (s, 1H), 4.24-4.13 (m, 1H), 4.11-4.02 (m, 1H), 2.87 (t, J=6.4 Hz, 2H).

### Example 76. (R)-7-(3-(2-(1H-Pyrazolo[3,4-b]pyrazin-3-yl)thiazol-4-yl)phenyl)-6,7-dihydro-5H-pyrrolo[1,2-a]imidazol-7-ol

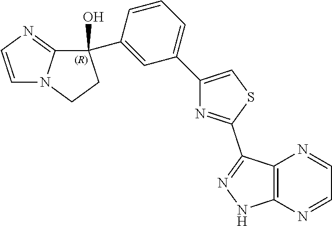

To a microwave vial containing 4-bromo-2-(1-tosyl-1H-pyrazolo[3,4-b]pyrazin-3-yl)thiazole (Intermediate 144, 200 mg, 0.458 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13. 194 mg, 0.595 mmol) and K₃PO₄ (290 mg, 1.37 mmol) was added 1,4-dioxane (12 mL), and H₂O (3 mL). The mixture was sparged with Ar for 5 minutes and then treated with Pd(dtbpf)Cl₂ (30 mg, 0.046 mmol). The mixture was sparged with Ar for another 5 minutes and then heated at 100° C. via microwave irradiation for 1 hour. After this time, the mixture was cooled to room temperature, filtered through a pad of diatomaceous earth, and the pad was washed with ethyl acetate (40 mL). The filtrate was concentrated to dryness under reduced pressure to give the product, which was purified by FCC (eluent: methylene chloride:methanol=1:0 to 1:1) to afford the product. The product was further purified by preparative HPLC using a Xtimate C18 150 mm×40 mm×10 μm column (eluent: 25% to 55% (v/v) CH₃CN and H₂O with 0.04% NH₃H₂O+10 mM NH₄HCO₃) to afford pure product. The product was suspended in water (10 mL), the mixture frozen using dry ice/acetone, and then lyophilized to dryness to afford the title compound (15.2 mg, 8%) as a yellow solid. LC-MS (ESI): mass calcd. For C₂₀H₁₅N₇OS 401.11 m/z found 402.0 [M+H]⁺. ¹H NMR (400 MHz, DMSO-d₆) δ 8.87-8.82 (m, 1H), 8.75-8.71 (m, 1H), 8.28-8.21 (m, 2H), 7.99 (d, J=7.3 Hz, 1H), 7.51-7.40 (m, 2H), 7.20 (s, 1H), 7.03 (s, 1H), 6.22 (s, 1H), 4.22-4.06 (m, 2H), 2.95-2.81 (m, 2H).

### Example 77. (R)-1-(3-(2-(5H-Pyrrolo[2,3-b]pyrazin-7-yl)thiazol-4-yl)phenyl)-1-(1H-imidazol-2-yl)ethan-1-ol

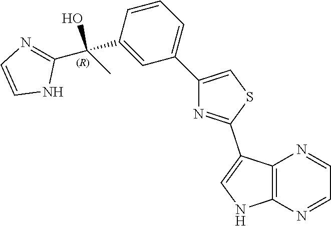

Step A. (R,S)-1-(3-(2-(5H-Pyrrolo[2,3-b]pyrazin-7-yl)thiazol-4-yl)phenyl)-1-(1H-imidazol-2-yl)ethanol. Sodium hydroxide (0.30 mL, 2.0 M in H₂O, 0.60 mmol) was added to a solution of 1-(1-tosyl-1H-imidazol-2-yl)-1-(3-(2-(5-tosyl-5H-pyrrolo[2,3-b]pyrazin-7-yl)thiazol-4-yl)phenyl)ethanol (Intermediate 139, Step D, 80 mg, 0.12 mmol) and 1,4-dioxane (3 mL). The resultant mixture was heated at 60° C. for 16 hours. After this time, the mixture was cooled to room temperature, and concentrated under reduced pressure to afford the product, which was purified by preparative HPLC using a Phenomenex Gemini NX-C18 75×30 mm×3 μm column (eluent: 8% to 38% (v/v) CH₃CN and H₂O with 0.2% FA) to afford pure product. The product was suspended in water (10 mL), the mixture frozen using dry ice/acetone, and then lyophilized to dryness to afford the title compound (27 mg, 60%) as a yellow solid. LC-MS (ESI): mass calcd. For C₂₀H₁₆N₆OS 388.11 m/z found 389.1 [M+H]⁺.

Step B. (R)-1-(3-(2-(5H-pyrrolo[2,3-b]pyrazin-7-yl)thiazol-4-yl)phenyl)-1-(1H-imidazol-2-yl)ethan-1-ol. The (R) and (5) enantiomers of (R,S)-1-(3-(2-(5H-Pyrrolo[2,3-b]pyrazin-7-yl)thiazol-4-yl)phenyl)-1-(1H-imidazol-2-yl)ethanol (54 mg, 0.14 mmol) were separated by SFC using a DAICEL CHIRALPAK AD 250 mm×30 mm×10 μm column. An isocratic elution was used throughout the purification which consisted of i-PrOH (containing 0.1% of 25% aq. NH₃): supercritical CO₂=50%: 50% to afford two enantiomers. The first eluting enantiomer, the title compound ((R)-1-(3-(2-(5H-pyrrolo[2,3-b]pyrazin-7-yl)thiazol-4-yl)phenyl)-1-(1H-imidazol-2-yl)ethan-1-ol), (8 mg, 15%) was isolated as a yellow solid. LC-MS (ESI): mass calcd. For C₂₀H₁₆N₆OS 388.11 m/z found 389.0 [M+H]⁺. ¹H NMR (400 MHz, DMSO-d₆) δ 12.79 (br s, 1H), 11.74 (br s, 1H), 8.64-8.56 (m, 2H), 8.44-8.40 (m, 1H), 8.20 (s, 1H), 7.98 (s, 1H), 7.92-7.85 (m, 1H), 7.41-7.35 (m, 2H), 6.85 (5, 2H), 6.12 (5, 1H), 1.90 (5, 3H). The second eluting enantiomer was (5)-1-(3-(2-(5H-pyrrolo[2,3-b]pyrazin-7-yl)thiazol-4-yl)phenyl)-1-(1H-imidazol-2-yl)ethan-1-ol (Example 78).

### Example 78. (S)-1-(3-(2-(5H-Pyrrolo[2,3-b]pyrazin-7-yl)thiazol-4-yl)phenyl)-1-(1H-imidazol-2-yl)ethan-1-ol

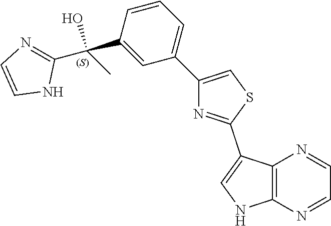

The chiral separation described in Example 77 also provided the title compound as the second eluting enantiomer (10 mg, 18%) as a yellow solid. LC-MS (ESI): mass calcd. For C₂₀H₁₆N₆OS 388.11 m/z found 389.0 [M+H]⁺. ¹H NMR (400 MHz, DMSO-d₆) δ 12.77 (br.s, 1H), 11.73 (br.s, 1H), 8.64-8.57 (m, 2H), 8.44-8.40 (m, 1H), 8.19 (s, 1H), 7.97 (s, 1H), 7.91-7.85 (m, 1H), 7.40-7.34 (m, 2H), 6.89 (s, 2H), 6.10 (s, 1H), 1.91 (s, 3H).

### Example 79. (S)-1-(3-(2-(1H-Pyrrolo[2,3-b]pyridin-3-yl)thiazol-4-yl)phenyl)-1-(1-methyl-1H-imidazol-2-yl)ethan-1-ol

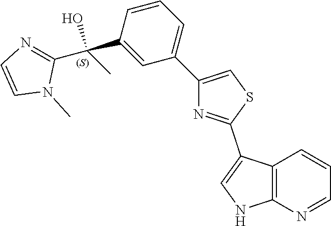

Step A. (R,S)-1-(3-(2-(1H-pyrrolo[2,3-b]pyridin-3-yl)thiazol-4-yl)phenyl)-1-(1-methyl-1H-imidazol-2-yl)ethan-1-ol. Sodium hydroxide (1.8 mL, 3.6 mmol, 2.0 M in H₂O) was added to a solution of 1-(1-methyl-1H-imidazol-2-yl)-1-(3-(2-(1-(phenylsulfonyl)-1H-pyrrolo[2,3-b]pyridin-3-yl)thiazol-4-yl)phenyl)ethanol (Intermediate 140, 410 mg, 0.689 mmol, 91% purity) and 1,4-dioxane (2 mL). The resultant mixture was heated at 60° C. for 16 hours. After this time, the reaction mixture was cooled to room temperature, and concentrated under reduced pressure to afford the product, which was triturated with methylene chloride (10 mL) and the resulting solid material was collected by filtration. The filter cake was dried under reduced pressure to give the product (250 mg, 90%) as a brown solid. LC-MS (ESI): mass calcd. For C₂₂H₁₉N₆OS 401.13 m/z, found 402.1 [M+H]⁺.

Step B. 1-(3-(2-(1H-pyrrolo[2,3-b]pyridin-3-yl)thiazol-4-yl)phenyl)-1-(1-methyl-1H imidazol-2-yl)ethan-1-ol 1-(3-(2-(1H-Pyrrolo[2,3-b]pyridin-3-yl)thiazol-4-yl)phenyl)-1-(1-methyl-1H-imidazol-2-yl)ethanol (250 mg, 0.623 mmol) was purified by SFC using a DAICEL CHIRALPAK AD 250 mm×30 mm×10 μm column. An isocratic elution was used throughout the purification which consisted of EtOH with 0.1% NH₃·H₂O: supercritical CO₂=45%: 55%. Two products were afforded, of which the second eluting enantiomer provided the titled compound a colorless solid (140 mg, 54%). LC-MS (ESI): mass calcd. For C₂₂H₁₉N₅OS 401.13 m/z, found 402.1 [M+H]⁺. ¹H NMR (400 MHz, DMSO-d₆) δ 12.23 (br s, 1H), 8.55 (d, J=7.8 Hz, 1H), 8.42-8.31 (m, 1H), 8.26 (s, 1H), 7.95-7.80 (m, 3H), 7.48-7.37 (m, 1H), 7.36-7.21 (m, 2H), 7.09 (s, 1H), 6.91 (s, 1H), 6.19 (s, 1H), 3.34 (s, 3H), 1.90 (s, 3H).

### Example 80. (R)-1-(3-(2-(1H-Pyrrolo[2,3-b]pyridin-3-yl)thiazol-4-yl)phenyl)-1-(1H-imidazol-2-yl)ethan-1-al

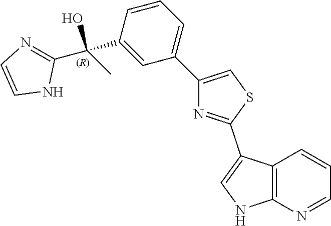

Step A. (R,S)-1-(3-(2-(1H-pyrrolo[2,3-b]pyridin-3-yl)thiazol-4-yl)phenyl)-1-(1H-imidazol-2-yl)ethan-1-ol. Sodium hydroxide (0.73 mL, 2.0 M in water, 1.5 mmol) was added to a 0° C. mixture of 1-(3-(2-(1-(phenylsulfonyl)-1H-pyrrolo[2,3-b]pyridin-3-yl)thiazol-4-yl)phenyl)-1-(1-tosyl-1H-imidazol-2-yl)ethanol (Intermediate 139, 200 mg, 0.293 mmol) and 1,4-dioxane (3 mL). The resultant mixture was heated at 60° C. for 16 hours. After this time, the mixture was cooled to room temperature and concentrated to afford the product, which was purified by HPLC over Phenomenex Gemini NX-C18, 75×30 mm×3 um column (eluent: 5% to 35% (v/v) CH₃CN and H₂O with 0.225% HCOOH) to afford pure product. The product was suspended in water (10 mL), the mixture frozen using dry ice/acetone, and then lyophilized to dryness to afford the racemic product, (R,S)-1-(3-(2-(1H-pyrrolo[2,3-b]pyridin-3-yl)thiazol-4-yl)phenyl)-1-(1H-imidazol-2-yl)ethan-1-ol, (80 mg, 71%) as a white solid.

Step B. (R)-1-(3-(2-(1H-pyrrolo[2,3-b]pyridin-3-yl)thiazol-4-yl)phenyl)-1-(1H-imidazol-2-yl)ethan-1-ol. The (R) and (S) enantiomers of (R,S)-1-(3-(2-(1H-pyrrolo[2,3-b]pyridin-3-yl)thiazol-4-yl)phenyl)-1-(1H-imidazol-2-yl)ethan-1-ol were separated by SFC using a DAICEL CHIRALCEL OD-H 250 mm×30 mm×5 μm column. An isocratic elution was used throughout the purification which consisted of EtOH: supercritical CO₂=70%: 30%. The first eluting enantiomer provided the title compound, (R)-1-(3-(2-(1H-pyrrolo[2,3-b]pyridin-3-yl)thiazol-4-yl)phenyl)-1-(1H-imidazol-2-yl)ethan-1-ol, as a white solid (5 mg, 6%). LC-MS (ESI): mass calcd. For C₂₁H₁₇N₅OS 387.12 m/z found 388.0 [M+H]⁺. ¹H NMR (400 MHz, DMSO-d₆) δ 12.30 (s, 1H), 11.76 (br.s, 1H), 8.67-8.59 (m, 1H), 8.40-8.33 (m, 1H), 8.29-8.22 (m, 1H), 8.12 (s, 1H), 7.93-7.80 (m, 2H), 7.48-7.42 (m, 1H), 7.42-7.36 (m, 1H), 7.35-7.28 (m, 1H), 7.04-6.87 (m, 2H), 6.11 (s, 1H), 1.89 (s, 3H).

### Example 81. (R)-7-(3-(2-(2-Methyl-5H-pyrrolo[2,3-b]pyrazin-7-yl)thiazol-4-yl)phenyl)-6,7-dihydro-5H-pyrrolo[1,2-a]imidazol-7-ol

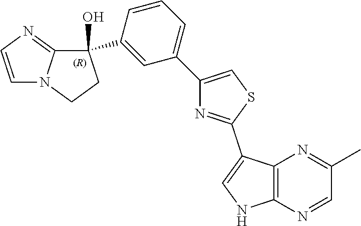

(R)-7-(3-(2-(2-Methyl-5-tosyl-5H-pyrrolo[2,3-b]pyrazin-7-yl)thiazol-4-yl)phenyl)-6,7-dihydro-5H-pyrrolo[1,2-a]imidazol-7-ol (Intermediate 141, 80 mg, 0.14 mmol) was added to a mixture of NaOH (0.44 mL, 2 M in water, 0.88 mmol) and 1,4-dioxane (6 mL). The reaction mixture was heated at 60° C. for 2 hours. After this time, the mixture was cooled to room temperature, adjusted to pH=7 with 1 M HCl, and concentrated under reduced pressure to give the product, which was purified by preparative HPLC using a Phenomenex Gemini-NX C18 75 mm×30 mm×3 μm (eluent: 30% to 60% (v/v) CH₃CN and H₂O with 0.04% NH₃H₂O+10 mM NH₄HCO₃) to afford the title compound (20.0 mg, 34%) as a yellow solid. LC-MS (ESI), mass calcd. For C₂₂H₁₈N₆OS 414.13 m/z found 415.0 [M+H]⁺. ¹H NMR (400 MHz, DMSO-d₆) δ 12.57 (br s, 1H), 8.49 (s, 1H), 8.27 (s, 1H), 8.15 (s, 1H), 7.96 (s, 1H), 7.94-7.90 (m, 1H), 7.44-7.36 (m, 2H), 7.16 (d, J=1.0 Hz, 1H), 6.99 (s, 1H), 6.14 (s, 1H), 4.22-3.97 (m, 2H), 2.94-2.75 (m, 2H), 2.64 (s, 3H).

### Example 82. (S)-7-(3-(2-(2-Methyl-5H-pyrrolo[2,3-b]pyrazin-7-yl)thiazol-4-yl)phenyl)-6,7-dihydro-5H-pyrrolo[1,2-a]imidazol-7-ol

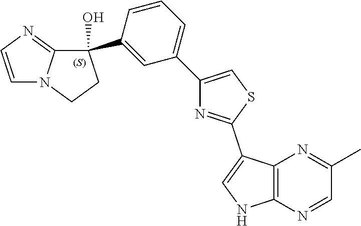

NaOH (0.53 mL, 2.0 M, 1.1 mmol) was added to a solution of (S)-7-(3-(2-(2-methyl-5-tosyl-5H-pyrrolo[2,3-b]pyrazin-7-yl)thiazol-4-yl)phenyl)-6,7-dihydro-5H-pyrrolo[1,2-a]imidazol-7-ol (Intermediate 142, 100 mg, 0.176 mmol) and 1,4-dioxane (6 mL). The resultant mixture was heated at 60° C. for 2 hours. The mixture was cooled to room temperature and concentrated under reduced pressure to give the product, which was further purified by preparative HPLC using a Phenomenex Gemini NX-C18 75×30 mm×3 μm column (eluent: 40% to 70% (v/v) CH₃CN and H₂O with 0.04% NH₃H₂O+10 mM NH₄HCO₃) to afford the title compound (12.6 mg, 17%) as a white solid. LCMS (ESI): mass calcd. for C₂₂H₁₈N₆OS 414.13 m/z, found 415.0 [M+H]⁺. ¹H NMR (400 MHz, DMSO-d₆) S 12.52 (br s, 1H), 8.51 (s, 1H), 8.30 (s, 1H), 8.18 (s, 1H), 7.98 (s, 1H), 7.97-7.94 (m, 1H), 7.46-7.38 (m, 2H), 7.21-7.18 (m, 1H), 7.03-7.00 (m, 1H), 6.20-6.16 (m, 1H), 4.20-4.05 (m, 2H), 2.94-2.78 (m, 2H), 2.67 (s, 3H).

### Example 83. (R)-7-(3-(2-(3-Methyl-5H-pyrrolo[2,3-b]pyrazin-7-yl)thiazol-4-yl)phenyl)-6,7-dihydro-5H-pyrrolo[1,2-a]imidazol-7-ol

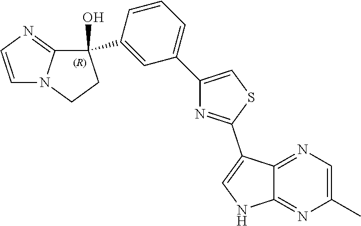

(R)-7-(3-(2-(3-methyl-5-tosyl-5H-pyrrolo[2,3-b]pyrazin-7-yl)thiazol-4-yl)phenyl)-6,7-dihydro-5H-pyrrolo[1,2-a]imidazol-7-ol (Intermediate 145, 100 mg, 0.176 mmol) was added to a mixture of NaOH (0.53 mL, 2 M in water, 1.1 mmol) and 1,4-dioxane (10 mL). The reaction mixture was heated at 60° C. for 1 hour. The mixture was cooled to room temperature and concentrated to dryness under reduced pressure to give the product, which was purified by preparative HPLC using a Boston Prime C18 150 mm×30 mm×5 μm column (eluent: 35% to 65% (v/v) CH₃CN and water (0.04% NH₃H₂O+10 mM NH₄HCO₃)) to afford pure product. The product was suspended in water (5 mL), the mixture frozen using dry ice/acetone, and then lyophilized to dryness to afford the title compound (36.7 mg, 50%) as a yellow solid. LC-MS (ESI): mass calcd. For C₂₂H₁₈N₆OS 414.13 m/z found 415.0 [M+H]⁺. ¹H NMR (400 MHz, DMSO-d₆) δ 12.49 (br s, 1H), 8.51 (s, 1H), 8.47 (s, 1H), 8.18 (s, 1H), 8.00 (s, 1H), 7.97-7.94 (m, 1H), 7.45-7.40 (m, 2H), 7.19 (d, J=1.1 Hz, 1H), 7.02 (d, J=0.9 Hz, 1H), 6.17 (s, 1H), 4.20-4.13 (m, 1H), 4.11-4.05 (m, 1H), 2.96-2.87 (m, 1H), 2.85-2.80 (m, 1H), 2.62 (s, 3H).

### Example 84. (S)-7-(3-(4-(1H-Pyrrolo[2,3-b]pyridin-3-yl)thiazol-2-yl)phenyl)-6,7-dihydro-5H-pyrrolo[1,2-a]imidazol-7-ol

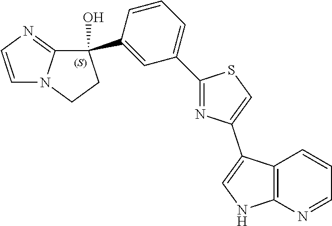

Sodium hydroxide (0.67 mL, 2.0 M in H₂O, 1.3 mmol) was added to a solution of (S)-7-(3-(4-(1-(phenylsulfonyl)-1H-pyrrolo[2,3-b]pyridin-3-yl)thiazol-2-yl)phenyl)-6,7-dihydro-5H-pyrrolo[1,2-a]imidazol-7-ol (Intermediate 146, 120 mg, 0.222 mmol) and 1,4-dioxane (6 mL). The resultant mixture was heated at 60° C. for 3 hours. After this time, the mixture was cooled to room temperature and concentrated to dryness under reduced pressure to give the product, which was purified by preparative HPLC using a Phenomenex Gemini-NX C18 75×30 mm×3 μm column (eluent: 27% to 57% (v/v) CH₃CN and H₂O with 0.04% NH₃H₂O+10 mM NH₄HCO₃) to afford pure product. The product was suspended in water (10 mL), the mixture frozen using dry ice/acetone, and then lyophilized to dryness to afford the title compound (28 mg, 32%) as a white solid. LCMS (ESI): mass calcd. for C₂₂H₁₇N₅OS 399.12 m/z, found 400.0 [M+H]⁺. ¹H NMR (400 MHz, DMSO-d₆) δ 11.99 (br s, 1H), 8.66-8.54 (m, 1H), 8.34-8.27 (m, 1H), 8.22 (s, 1H), 8.08 (s, 1H), 8.00-7.95 (m, 1H), 7.92 (s, 1H), 7.55-7.49 (m, 2H), 7.25-7.19 (m, 2H), 7.07 (s, 1H), 6.30 (br s, 1H), 4.22-4.04 (m, 2H), 2.90-2.81 (m, 2H).

### Example 85. (R)-3-(3-(2-(1H-Pyrrolo[2,3-b]pyridin-3-yl)thiazol-4-yl)phenyl)-2,3-dihydrofuro[3,2-b]pyridin-3-ol

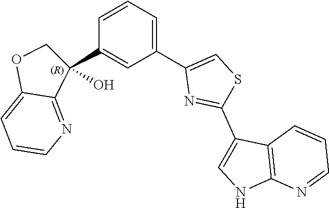

NaOH (0.60 mL, 2.0 M in water, 1.2 mmol) was added to a solution of (R)-3-(3-(2-(1-(phenylsulfonyl)-1H-pyrrolo[2,3-b]pyridin-3-yl)thiazol-4-yl)phenyl)-2,3-dihydrofuro[3,2-b]pyridin-3-ol (Intermediate 148, 110 mg, 0.199 mmol) and 1,4-dioxane (6 mL). The resultant mixture was heated at 60° C. for 1 hour. The mixture was cooled to room temperature and concentrated to dryness under reduced pressure to give the product, which was purified by preparative HPLC using a Phenomenex Gemini-NX C18 75×30 mm×3 μm column (eluent: 30% to 60% (v/v) CH₃CN and H₂O with 0.04% NH₃H₂O+10 mM NH₄HCO₃) to afford the title compound (40.4 mg, 48%) as a white solid. LCMS (ESI): mass calcd. for C₂₃H₁₆N₄O₂S 412.10 m/z, found 413.0 [M+H]⁺. ¹H NMR (400 MHz, DMSO-d₆) δ 12.30 (br s, 1H), 8.57-8.50 (m, 1H), 8.39-8.33 (m, 1H), 8.30-8.23 (m, 1H), 8.18-8.11 (m, 2H), 8.00-7.93 (m, 2H), 7.51-7.40 (m, 3H), 7.36-7.25 (m, 2H), 6.56 (s, 1H), 4.81-4.63 (m, 2H).

### Example 86. (R)-3-(3-(2-(5H-Pyrrolo[2,3-b]pyrazin-7-yl)thiazol-4-yl)phenyl)-2,3-dihydrofuro[3,2-b]pyridin-3-ol

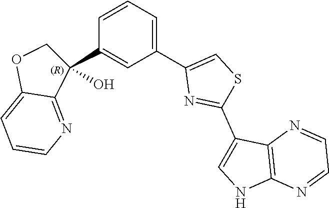

NaOH (0.50 mL, 2.0 M in water, 1.0 mmol) was added to a solution of (R)-3-(3-(2-(5-tosyl-5H-pyrrolo[2,3-b]pyrazin-7-yl)thiazol-4-yl)phenyl)-2,3-dihydrofuro[3,2-b]pyridin-3-ol (Intermediate 150, 95 mg, 0.17 mmol) and 1,4-dioxane (6 mL). The resultant mixture was heated at 60° C. for 1 hour. The mixture was cooled to room temperature and concentrated to dryness under reduced pressure to give the product, which was purified by preparative HPLC using a Phenomenex Gemini-NX C18 75×30 mm×3 μm column (eluent: 30% to 60% (v/v) CH₃CN and H₂O with 0.05% NH₃H₂O+10 mM NH₄HCO₃) to afford the title compound (27.6 mg, 40%) as a white solid. LCMS (ESI): mass calcd. for C₂₂H₁₅N₅O₂S 413.09 m/z, found 414.0 [M+H]⁺. ¹H NMR (400 MHz, DMSO-d₆) δ 8.60 (s, 1H), 8.57 (d, J=2.7 Hz, 1H), 8.38 (d, J=2.4 Hz, 1H), 8.20-8.15 (m, 1H), 8.12-8.09 (m, 1H), 8.02 (s, 1H), 8.00-7.95 (m, 1H), 7.47-7.40 (m, 2H), 7.39-7.34 (m, 1H), 7.31-7.25 (m, 1H), 6.52 (br s, 1H), 4.78-4.67 (m, 2H).

### Example 87. (R)-7-(3-(2-(4-Methyl-1H-pyrrolo[2,3-b]pyridin-3-yl)thiazol-4-yl)phenyl)-6,7-dihydro-5H-pyrrolo[1,2-a]imidazol-7-ol

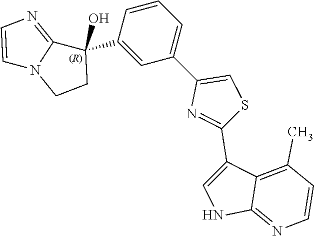

(R)-7-(3-(2-(4-Methyl-1-tosyl-1H-pyrrolo[2,3-b]pyridin-3-yl)thiazol-4-yl)phenyl)-6,7-dihydro-5H-pyrrolo[1,2-a]imidazol-7-ol (Intermediate 151, 150 mg, 0.264 mmol) was added to a mixture of NaOH (0.8 mL, 2 M in water, 1.6 mmol) and 1,4-dioxane (6 mL). The reaction mixture was heated at 60° C. for 2 hours. The mixture was cooled to room temperature and adjusting to pH=7 with 6 M HCl. The reaction mixture was concentrated to dryness under reduced pressure to give the product, which was purified by preparative HPLC using a Phenomenex Gemini-NX C18 75 mm×30 mm×3 μm (eluent: 25% to 55% (v/v) CH₃CN and H₂O with 0.04% NH₃H₂O+10 mM NH₄HCO₃) to afford the title compound (22.5 mg, 20%) as a brown solid. LC-MS (ESI): mass calcd. For C₂₃H₁₉N₅OS 413.13 m/z found 414.1 [M+H]⁺. ¹H NMR (400 MHz, DMSO-d₆) δ 12.23 (br s, 1H), 8.18 (d, J=4.8 Hz, 1H), 8.12 (s, 1H), 8.00 (d, J=2.8 Hz, 1H), 7.96 (s, 1H), 7.91-7.87 (m, 1H), 7.49-7.40 (m, 2H), 7.18 (s, 1H), 7.03-6.98 (m, 2H), 6.16 (s, 1H), 4.20-4.11 (m, 1H), 4.09-4.00 (m, 1H), 2.88-2.79 (m, 2H), 2.75 (s, 3H).

### Example 88. (R)-7-(3-(2-(7H-pyrrolo[2,3-c]pyridazin-5-yl)thiazol-4-yl)phenyl)-6,7-dihydro-5H-pyrrolo[1,2-a]imidazol-7-ol

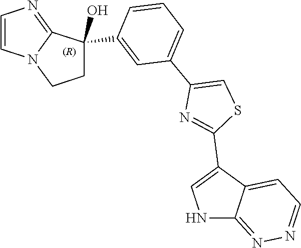

Sodium hydroxide (0.65 mL, 2.0 M, 1.3 mmol) was added to a solution of (R)-7-(3-(2-(7-tosyl-7H-pyrrolo[2,3-c]pyridazin-5-yl)thiazol-4-yl)phenyl)-6,7-dihydro-5H-pyrrolo[1,2-a]imidazol-7-ol (Intermediate 152, 120 mg, 0.216 mmol) and 1,4-dioxane (6 mL). The resultant mixture was heated at 60° C. for 1 hour. After this time, the mixture was cooled to room temperature and concentrated to dryness under reduced pressure to give the product, which was purified by preparative HPLC using a Xtimate C18 250×80 mm×10 μm column (eluent: 20% to 50% (v/v) CH₃CN and H₂O with 0.04% NH₃H₂O+10 mM NH₄HCO₃) to afford pure product. The product was suspended in water (10 mL), the mixture frozen using dry ice/acetone, and then lyophilized to dryness to afford the title compound (12.6 mg, 15%) as a brown solid. LCMS (ESI): mass calcd. for C₂₁H₁₅N₆OS 400.11 m/z, found 401.0 [M+H]⁺. ¹H NMR (400 MHz, DMSO-d₆) δ 13.06 (br s, 1H), 9.10 (d, J=5.6 Hz, 1H), 8.69 (s, 1H), 8.41 (d, J=5.6 Hz, 1H), 8.22 (s, 1H), 8.01-7.94 (m, 2H), 7.50-7.43 (m, 2H), 7.24 (s, 1H), 7.10 (s, 1H), 6.19 (s, 1H), 4.22-4.13 (m, 1H), 4.11-4.03 (m, 1H), 2.86 (t, J=6.4 Hz, 2H).

### Example 89. (R)-3-(3-(2-(1H-Pyrrolo[2,3-b]pyridin-3-yl)thiazol-4-yl)phenyl)-3-hydroxy-1,3-dihydro-2H-pyrrolo[3,2-b]pyridin-2-one

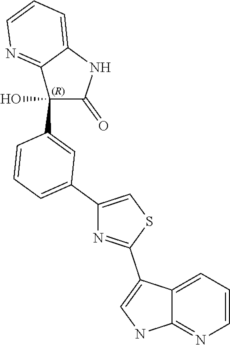

Step A. (R,S)-3-(3-(2-(1H-Pyrrolo[2,3-b]pyridin-3-yl)thiazol-4-yl)phenyl)-3-hydroxy-1H-pyrrolo[3,2-b]pyridin-2(3H)-one. NaOH (0.5 mL, 2 M in water, 1.0 mmol) was added to a solution of 3-hydroxy-3-(3-(2-(1-(phenylsulfonyl)-1H-pyrrolo[2,3-b]pyridin-3-yl)thiazol-4-yl)phenyl)-1H-pyrrolo[3,2-b]pyridin-2(3H)-one (Intermediate 153, 200 mg, 0.35 mmol) and 1,4-dioxane (6 mL). The mixture was heated at 60° C. for 1 hour. The mixture was cooled to room temperature and the solvent was removed under reduced pressure to give the product, which was purified by preparative HPLC using a Boston Prime C18 150×30 mm×5 μm column (eluent: 30% to 60% (v/v) CH₃CN and H₂O with 0.05% NH₃H₂O+10 mM NH₄HCO₃) to afford the title compound (45 mg, 30%) as a white solid. LC-MS (ESI): mass calcd. For C₂₃H₁₅N₅O₂S 425.10 m/z found 426.3 [M+H]⁺.

Step B. (R)-3-(3-(2-(1H-Pyrrolo[2,3-b]pyridin-3-yl)thiazol-4-yl)phenyl)-3-hydroxy-1,3-dihydro-2H-pyrrolo[3,2-b]pyridin-2-one. The (R) and (S) enantiomers of (R,S)-3-(3-(2-(1H-pyrrolo[2,3-b]pyridin-3-yl)thiazol-4-yl)phenyl)-3-hydroxy-1H-pyrrolo[3,2-b]pyridin-2(3H)-one (45 mg, 0.11 mmol) were separated by SFC over DAICEL CHIRALCEL OD 250 mm×30 mm×10 μm column (isocratic elution: Et₀H (containing 0.1% of 25% aq. NH₃): supercritical CO₂=45%: 55% (v/v)) to afford two enantiomers. The first eluting enantiomer, (R)-3-(3-(2-(1H-pyrrolo[2,3-b]pyridin-3-yl)thiazol-4-yl)phenyl)-3-hydroxy-1,3-dihydro-2H-pyrrolo[3,2-b]pyridin-2-one, (12.6 mg, 27%) was obtained as a white solid. LC-MS (ESI): mass calcd. For C₂₃H₁₅N₅O₂S 425.10 m/z found 426.0 [M+H]⁺. ¹H NMR (400 MHz, DMSO-d₆) δ 8.47 (dd, J=1.5, 7.9 Hz, 1H), 8.37 (dd, J=1.6, 4.6 Hz, 1H), 8.27 (s, 1H), 8.18 (dd, J=1.9, 4.4 Hz, 1H), 7.98-7.89 (m, 3H), 7.49-7.43 (m, 1H), 7.41-7.30 (m, 4H), 6.91 (br s, 1H). The second eluting enantiomer, (S)-3-(3-(2-(1H-pyrrolo[2,3-b]pyridin-3-yl)thiazol-4-yl)phenyl)-3-hydroxy-1,3-dihydro-2H-pyrrolo[3,2-b]pyridin-2-one, (Example 90) was also obtained.

### Example 90. (S)-3-(3-(2-(1H-Pyrrolo[2,3-b]pyridin-3-yl)thiazol-4-yl)phenyl)-3-hydroxy-1,3-dihydro-2H-pyrrolo[3,2-b]pyridin-2-one

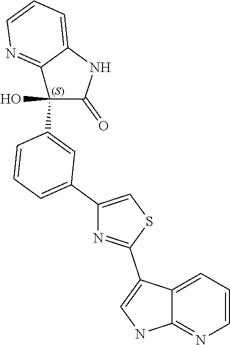

The second eluting enantiomer obtained in Example 89, the title compound, (11.0 mg, 24%) was obtained as a white solid. LC-MS (ESI): mass calcd. For C₂₃H₁₅N₅O₂S 425.10 m/z found 426.0 [M+H]⁺. ¹H NMR (400 MHz, DMSO-d₆) δ 8.47-12.28 (br s, 1H), 10.73 (br s, 1H), 8.47 (dd, J=1.5, 8.0 Hz, 1H), 8.37 (dd, J=1.5, 4.8 Hz, 1H), 8.26 (s, 1H), 8.18 (dd, J=1.9, 4.5 Hz, 1H), 7.98-7.89 (m, 3H), 7.49-7.42 (m, 1H), 7.39-7.30 (m, 4H), 6.91 (s, 1H).

### Example 91. (R)-3-(3-(2-(1H-Pyrrolo[2,3-b]pyridin-3-yl)thiazol-4-yl)phenyl)-3-hydroxy-1-methyl-1,3-dihydro-2H-pyrrolo[3,2-b]pyridin-2-one

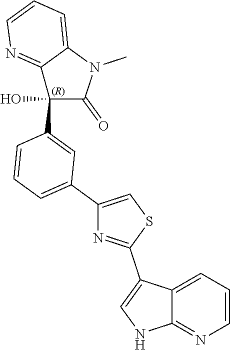

Sodium hydroxide (0.2 mL, 2.0 M in H₂O, 0.4 mmol) was added to a solution of (R)-3-hydroxy-1-methyl-3-(3-(2-(1-(phenylsulfonyl)-1H-pyrrolo[2,3-b]pyridin-3-yl)thiazol-4-yl)phenyl)-1H-pyrrolo[3,2-b]pyridin-2(3H)-one (Intermediate 156, 70 mg, 0.12 mmol) and 1,4-dioxane (10 mL). The reasultant mixture was heated at 60° C. for 1 hour. After this time, the mixture was cooled to room temperature and concentrated to give the product, which was purified by preparative HPLC using a Boston Prime C18 150×30 mm×5 μm column (eluent: 35% to 65% (v/v) CH₃CN and water (0.05% NH₃H₂O+10 mM NH₄HCO₃)) to afford the title compound (12 mg, 25%) as a brown solid. LC-MS (ESI): mass calcd. For C₂₄H₁₇N₅O₂S 439.11 m/z found 439.9 [M+H]⁺. 1H NMR (400 MHz, DMSO-d₆) δ 12.31 (s, 1H), 8.48 (s, 1H), 8.42-8.32 (m, 1H), 8.27 (s, 1H), 8.25-8.20 (m, 1H), 8.04-7.85 (m, 3H), 7.66-7.53 (m, 1H), 7.49-7.40 (m, 2H), 7.40-7.35 (m, 1H), 7.35-7.22 (m, 1H), 6.99 (br s, 1H), 3.24 (s, 3H).

### Example 92. (S)-3-(3-(2-(1H-Pyrrolo[2,3-b]pyridin-3-yl)thiazol-4-yl)phenyl)-3-hydroxy-1-methyl-1,3-dihydro-2H-pyrrolo[3,2-b]pyridin-2-one

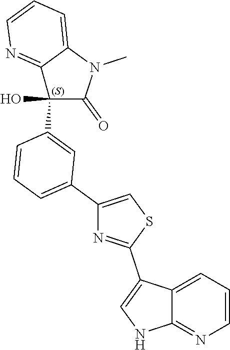

Sodium hydroxide (0.21 mL, 2.0 M in H₂O, 0.42 mmol) was added to a solution of (S)-3-hydroxy-1-methyl-3-(3-(2-(1-(phenylsulfonyl)-1H-pyrrolo[2,3-b]pyridin-3-yl)thiazol-4-yl)phenyl)-1H-pyrrolo[3,2-b]pyridin-2(3H)-one (Intermediate 157, 80 mg, 0.14 mmol) and 1,4-dioxane (10 mL). The reasultant mixture was heated at 60° C. for 1 hour. After this time, the mixture was cooled to room temperature and concentrated under reduced pressure to give the product, which was purified by preparative HPLC using a Boston Prime C18 150×30 mm×5 μm column (eluent: 35% to 65% (v/v) water (0.05% NH₃H₂O+10 mM NH₄HCO₃)—CH₃CN to afford the title compound (22.0 mg, 36%) as a white solid. LC-MS (ESI): mass calcd. For C₂₄H₁₇N₅O₂S 439.11 m/z found 440.0 [M+H]⁺. 1H NMR (400 MHz, DMSO-d₆) δ 13.48 (s, 1H), 7.43-7.34 (m, 2H), 7.31 (s, 1H), 7.11 (s, 1H), 7.08-7.00 (m, 2H), 4.81 (s, 1H), 3.59 (d, J=2.9 Hz, 1H), 2.61 (s, 3H), 2.30 (d, J=9.5 Hz, 1H), 2.07-1.99 (m, 1H), 1.86 (d, J=9.5 Hz, 2H), 1.26-1.19 (m, 1H), 0.99 (s, 1H).

### Example 93. (R)-3-(3-(5-(1H-Pyrrolo[2,3-b]pyridin-3-yl)furan-3-yl)phenyl)-3-hydroxy-1-methylpyrrolidin-2-one

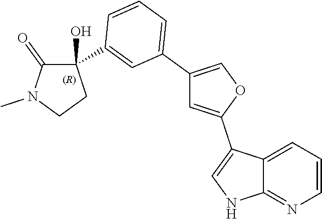

Sodium hydroxide (0.46 mL, 2.0 M in H₂O, 0.92 mmol) was added to a solution of (R)-3-hydroxy-1-methyl-3-(3-(5-(1-tosyl-1H-pyrrolo[2,3-b]pyridin-3-yl)furan-3-yl)phenyl)pyrrolidin-2-one (Intermediate 158, 80 mg, 0.15 mmol) and 1,4-dioxane (5 mL). The resultant mixture was heated at 70° C. for 1 hour. After this time, the mixture was cooled to room temperature. The solvent was removed under reduced pressure to give the product, which was purified by preparative HPLC using a Boston Prime C18 150×30 mm×5 μm column (eluent: 35% to 65% (v/v) CH₃CN and water (0.05% NH₃H₂O+10 mM NH₄HCO₃)) to afford the title compound (10.0 mg, 17%) as a white solid. LC-MS (ESI): mass calcd. For C₂₂H₁₉N₃O₃ 373.14 m/z found 374.0 [M+H]⁺. ¹H NMR (400 MHz, DMSO-d₆) δ 11.76 (s, 1H), 8.37 (d, J=8.0 Hz, 1H), 8.33-8.29 (m, 1H), 8.05 (d, J=0.8 Hz, 1H), 7.84 (s, 1H), 7.69 (s, 1H), 7.59-7.54 (m, 1H), 7.40-7.34 (m, 1H), 7.28 (d, J=7.9 Hz, 1H), 7.22-7.17 (m, 1H), 7.09 (s, 1H), 5.73 (d, J=1.9 Hz, 1H), 3.50-3.44 (m, 1H), 3.40-3.35 (m, 1H), 2.87 (s, 3H), 2.45-2.39 (m, 1H), 2.33-2.29 (m, 1H).

### Example 94. (R)-3-(3-(2-(4,5-Difluoro-1H-pyrrolo[2,3-b]pyridin-3-yl)thiazol-4-yl)phenyl)-3-hydroxy-1-methylpyrrolidin-2-one

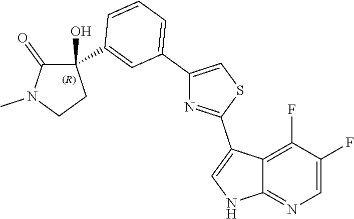

LiOH·H₂O (59 mg, 1.4 mmol) was added to a solution of (R)-3-(3-(2-(4,5-difluoro-1-tosyl-1H-pyrrolo[2,3-b]pyridin-3-yl)thiazol-4-yl)phenyl)-3-hydroxy-1-methylpyrrolidin-2-one (Intermediate 159, 270 mg, 0.465 mmol), 1,4-dioxane (8 mL), and H₂O (2 mL). The reaction mixture was heated at 70° C. for 2 hours. The mixture was cooled to room temperature and concentrated to dryness under reduced pressure to give the product, which was purified by preparative HPLC using a Phenomenex Gemini NX-C18 75 mm×30 mm, 3 μm (eluent: 30% to 60% (v/v) CH₃CN and water (0.05% NH₃H₂O+10 mM NH₄HCO₃)) to afford a still-impure product (150 mg) as a white solid. The resulting product was further purified by SFC using a DAICEL CHIRALPAK AS 250 mm×30 mm, 10 μm (isocratic elution: EtOH, containing 0.1% of 25% aq. NH₃): supercritical CO₂=40%: 60% (v/v)) to afford the title compound (125 mg, 63%) as a white solid. LC-MS (ESI): mass calcd. For C₂₁H₁₆F₂N₄O₂S 426.10 m/z, found 427.0 [M+H]⁺. ¹H NMR (400 MHz, DMSO-d₆) δ 8.48-8.41 (m, 1H), 8.24 (s, 1H), 8.07 (s, 1H), 7.95-7.86 (m, 2H), 7.46-7.36 (m, 2H), 5.74 (br s, 1H), 3.52-3.44 (m, 1H), 3.43-3.35 (m, 1H), 2.92-2.82 (m, 3H), 2.45-2.38 (m, 1H), 2.37-2.30 (m, 1H).

### Example 95. (R)-3-(3-(2-(5-Chloro-4-fluoro-1H-pyrrolo[2,3-b]pyridin-3-yl)thiazol-4-yl)phenyl)-3-hydroxy-1-methylpyrrolidin-2-one

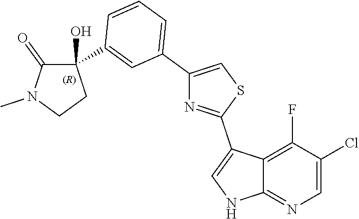

To a microwave vial containing 4-bromo-2-(5-chloro-4-fluoro-1H-pyrrolo[2,3-b]pyridin-3-yl)thiazole (Intermediate 160, 60.0 mg, 0.163 mmol, as HCl salt), (R)-3-hydroxy-1-methyl-3-(3-(4,4,5,5-tetramethyl-1,3,2-dioxaborolan-2-yl)phenyl)pyrrolidin-2-one (Intermediate 16, 77.4 mg, 0.244 mmol), and K₂CO₃ (67.4 mg, 0.488 mmol) was added 1,4-dioxane (8 mL) and H₂O (2 mL). The resultant mixture was purged with Ar for 5 minutes and then treated with Pd(dppf)Cl₂ (11.9 mg, 0.016 mmol). The resultant mixture was purged with Ar for another 5 minutes and then heated at 100° C. via microwave irradiation for 1 h. The reaction was cooled to room temperature and concentrated to dryness under reduced pressure. The product was initially purified by silica gel chromatography (eluent: dichloromethane:MeOH=10:1) and then further purified by preparative HPLC using a Phenomenex Gemini NX-C18 750×30 mm×4 μm column (eluent: 33% to 63% (v/v) CH₃CN and H₂O with 0.05% NH₃H₂O+10 mM NH₄HCO₃) to afford the title compound (40 mg, 55%) as a white solid. LC-MS (ESI): mass calcd. for C₂₁H₁₆ClFN₄O₂S 442.07 m/z, found 442.9 [M+H]⁺. ¹H NMR (400 MHz, DMSO-d₆) δ 12.80 (br s, 1H), 8.45 (d, J=8.8 Hz, 1H), 8.34 (s, 1H), 8.10-7.99 (m, 2H), 7.93 (d, J=7.5 Hz, 1H), 7.50-7.33 (m, 2H), 6.09 (s, 1H), 3.54-3.40 (m, 2H), 2.88 (s, 3H), 2.44-2.24 (m, 2H).

### Example 96. (R)-7-(3-(2-(4,5-Difluoro-1H-pyrrolo[2,3-b]pyridin-3-yl)thiazol-4-yl)phenyl)-6,7-dihydro-5H-pyrrolo[1,2-a]imidazol-7-ol

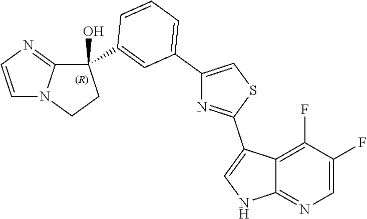

LiOH·H₂O (64 mg, 1.5 mmol) was added to a solution of (R)-7-(3-(2-(4,5-difluoro-1-tosyl-1H-pyrrolo[2,3-b]pyridin-3-yl)thiazol-4-yl)phenyl)-6,7-dihydro-5H-pyrrolo[1,2-a]imidazol-7-ol (Intermediate 161, 300 mg, 0.509 mmol), 1,4-dioxane (4 mL), and H₂O (1 mL). The reaction mixture was heated at 70° C. for 1 hour. The mixture was cooled to room temperature and concentrated to dryness under reduced pressure to give the product, which was purified by preparative HPLC using a Boston Prime C18 150×30 mm, 5 μm (eluent: 40% to 70% (v/v) CH₃CN and water (0.05% NH₃H₂O+10 mM NH₄HCO₃)) to afford pure product. The product was suspended in water (10 mL), the mixture frozen using dry ice/acetone, and then lyophilized to dryness to afford the title compound (73.1 mg, 33%) as a white solid. LC-MS (ESI): mass calcd. For C₂₂H₁₅F₂N₅OS 435.10 m/z, found 436.0 [M+H]⁺. ¹H NMR (400 MHz, DMSO-d₆) δ 12.80 (br s, 1H), 8.56-8.49 (m, 1H), 8.35 (s, 1H), 8.17 (s, 1H), 8.01-7.93 (m, 2H), 7.46-7.40 (m, 2H), 7.20-7.16 (m, 1H), 7.02 (s, 1H), 6.17 (s, 1H), 4.21-4.03 (m, 2H), 2.92-2.79 (m, 2H).

### Example 97. (R)-7-(3-(2-(5-Chloro-4-fluoro-1H-pyrrolo[2,3-b]pyridin-3-yl)thiazol-4-yl)phenyl)-6,7-dihydro-5H-pyrrolo[1,2-a]imidazol-7-ol

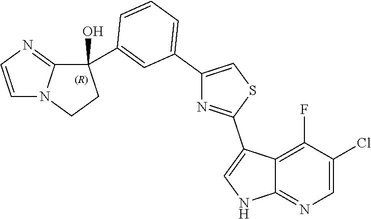

To a microwave vial containing 4-Bromo-2-(5-chloro-4-fluoro-1H-pyrrolo[2,3-b]pyridin-3-yl)thiazole (Intermediate 160, 80 mg, 0.24 mmol), (R)-7-(3-(4,4,5,5-tetramethyl-1,3,2-dioxaborolan-2-yl)phenyl)-6,7-dihydro-5H-pyrrolo[1,2-a]imidazol-7-ol (Intermediate 13 235 mg), and K₂CO₃ (99.7 mg, 0.721 mmol) were added 1,4-dioxane (8 mL) and H₂O (2 mL). The resultant mixture was sparged with Ar for 5 minutes and then treated with Pd(dppf)Cl₂ (35.2 mg, 0.048 mmol). The mixture was sparged with Ar for another 5 minutes and then heated at 100° C. via microwave irradiation for 1 h. The reaction was concentrated to dryness under reduced pressure and initially purified by FCC (eluent: dichloromethane:MeOH=10:1). The product was further purified by preparative HPLC using a Boston Prime C18 150×30 mm×5 μm column (eluent: 55% to 85% (v/v) CH₃CN and H₂O with 0.05% NH₃H₂O+10 mM NH₄HCO₃) to afford pure product. The product was suspended in water (10 mL), the mixture frozen using dry ice/acetone, and then lyophilized to dryness to afford the title compound (39 mg, 34%) as a white solid. LC-MS (ESI): mass calcd. for C₂₂H₁₅ClFN₅OS 451.07 m/z, found 452.0 [M+H]⁺. ¹H NMR (400 MHz, DMSO-d₆) δ 12.89 (br s, 1H), 8.45 (d, J=9.0 Hz, 1H), 8.33 (s, 1H), 8.14 (s, 1H), 7.99 (s, 1H), 7.97-7.91 (m, 1H), 7.48-7.38 (m, 2H), 7.18 (d, J=1.1 Hz, 1H), 7.05-6.98 (m, 1H), 7.05-6.97 (m, 1H), 6.17 (s, 1H), 4.22-4.13 (m, 1H), 4.12-4.03 (m, 1H), 2.93-2.79 (m, 2H).

### Example 98. (R)-4-(3-(3-Hydroxy-1-methyl-2-oxopyrrolidin-3-yl)phenyl)-2-(1H-pyrrolo[2,3-b]pyridin-3-yl)thiazole-5-carbonitrile

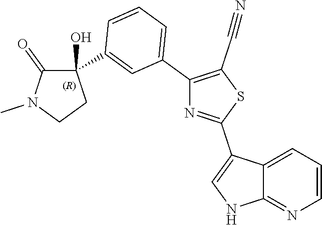

Sodium hydroxide (3.0 mL, 6.0 mmol, 2 M) was added to a mixture of (R)-4-(3-(3-hydroxy-1-methyl-2-oxopyrrolidin-3-yl)phenyl)-2-(1-tosyl-1H-pyrrolo[2,3-b]pyridin-3-yl)thiazole-5-carbonitrile (Intermediate 162, 680 mg, 1.19 mmol) in 1,4-dioxane (12 mL). The mixture was stirred for 2 h at 60° C. After this time, the mixture was concentrated to dryness under reduced pressure and purified by silica gel chromatography (petroleum ether:ethyl acetate=1:0 to 0:1 to CH₂Cl₂:MeOH=9:1) to afford the title product (450 mg) as a brown solid. The product was further purified by preparative HPLC using a Phenomenex Gemini NX-C18 75×30 mm×3 μm column (eluent: 27% to 57% (v/v) ACN and water (0.05% NH₃H₂O+10 mM NH₄HCO₃)) to afford the title compound (85.7 mg, 17%) as a yellow solid. LCMS (ESI): mass calcd. for C₂₂H₁₇N₅O₂S 415.11 m/z, found 416.2 [M+H]⁺. ¹H NMR (400 Hz, DMSO-d₆) δ 3.05-11.53 (m, 1H), 8.67-8.61 (m, 1H), 8.60 (s, 1H), 8.45-8.38 (m, 1H), 8.17 (s, 1H), 8.12-8.05 (m, 1H), 7.67-7.53 (m, 2H), 7.39-7.30 (m, 1H), 6.24 (s, 1H), 3.54-3.49 (m, 2H), 2.90 (s, 3H), 2.44-2.27 (m, 2H) (5, 3H).

### Example 99. (R)-3-(3-(5-Fluoro-2-(1H-pyrrolo[2,3-b]pyridin-3-yl)thiazol-4-yl)phenyl)-3-hydroxy-1-methylpyrrolidin-2-one

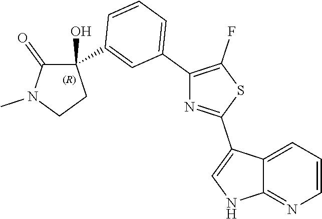

Step A. 3-(3-(5-Fluoro-2-(1H-pyrrolo[2,3-b]pyridin-3-yl)thiazol-4-yl)phenyl)-3-hydroxy-1-methylpyrrolidin-2-one. Sodium hydroxide (0.90 mL, 2.0 M in H₂O, 1.8 mmol) was added to a solution of 3-(3-(5-fluoro-2-(1-tosyl-1H-pyrrolo[2,3-b]pyridin-3-yl)thiazol-4-yl)phenyl)-1-methyl-2-oxopyrrolidin-3-yl acetate (Intermediate 163, 180 mg) and 1,4-dioxane (3 mL). The resultant mixture was heated at 60° C. for 2 hours. After this time, the mixture was concentrated to dryness to afford the product, which was purified by silica gel chromatography (eluent: methylene chloride:MeOH=1:0 to 9:1) to give the title compound (65 mg, 46%) as a yellow solid. LC-MS (ESI): mass calcd. for C₂₁H₁₇FN₄O₂S 408.11 m/z found 409.0 [M+H]⁺.

Step B. (R)-3-(3-(5-fluoro-2-(1H-pyrrolo[2,3-b]pyridin-3-yl)thiazol-4-yl)phenyl)-3-hydroxy-1-methylpyrrolidin-2-one. The (R) and (S) enantiomers of 3-(3-(5-Fluoro-2-(1H-pyrrolo[2,3-b]pyridin-3-yl)thiazol-4-yl)phenyl)-3-hydroxy-1-methylpyrrolidin-2-one (90 mg, 0.22 mmol) was purified by prep. HPLC using a Phenomenex Gemini NX-C18 75×30 mm×3 μm column (eluent: 32% to 62% (v/v) CH₃CN and H₂O with 0.05% ammonia hydroxide+10 mM NH₄HCO₃) to afford pure product (40 mg), which was further purified by SFC over Phenomenex-Cellulose-2 250×30 mm×10 μm column. An isocratic elution was used throughout the purification which consisted of EtOH: supercritical CO₂=55%: 45%. The second eluting enantiomer afforded the title compound (14.8 mg, 37%) as a white solid. as a white solid. LC-MS (ESI): mass calcd. for C₂₁H₁₇FN₄O₂S 408.11 m/z found 409.1 [M+H]⁺. ¹H NMR (400 MHz, DMSO-d₆) δ 12.35 (br s, 1H), 8.67-8.54 (m, 1H), 8.41-8.34 (m, 1H), 8.28 (s, 1H), 8.01 (s, 1H), 7.86 (d, J=7.7 Hz, 1H), 7.57-7.46 (m, 1H), 7.38 (d, J=7.7 Hz, 1H), 7.33-7.24 (m, 1H), 6.17 (s, 1H), 3.54-3.43 (m, 2H), 2.89 (s, 3H), 2.39-2.24 (m, 2H). Also obtained from the chiral separation from Step B was the first eluting enantiomer, (S)-3-(3-(5-fluoro-2-(1H-pyrrolo[2,3-b]pyridin-3-yl)thiazol-4-yl)phenyl)-3-hydroxy-1-methylpyrrolidin-2-one (18.3 mg, 45%). LC-MS (ESI): mass calcd. for C₂₁H₁₇FN₄O₂S 408.11 m/z found 409.0 [M+H]⁺. ¹H NMR (400 MHz, DMSO-d₆) δ 12.35 (br s, 1H), 8.67-8.56 (m, 1H), 8.41-8.33 (m, 1H), 8.28 (s, 1H), 8.01 (s, 1H), 7.86 (d, J=7.5 Hz, 1H), 7.56-7.45 (m, 1H), 7.38 (d, J=7.9 Hz, 1H), 7.32-7.24 (m, 1H), 6.17 (s, 1H), 3.52-3.42 (m, 2H), 2.89 (s, 3H), 2.36-2.24 (m, 2H).

### Example 100. (R)-3-(3-(2-(1H-Pyrazolo[3,4-b]pyridin-3-yl)thiazol-4-yl)phenyl)-3-hydroxy-1-methylpyrrolidin-2-one

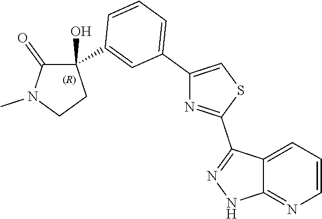

To a 1,4-dioxane (2 mL) solution of (3R)-3-hydroxy-1-methyl-3-(3-(2-(1-(tetrahydro-2H-pyran-2-yl)-1H-pyrazolo[3,4-b]pyridin-3-yl)thiazol-4-yl)phenyl)pyrrolidin-2-one (160 mg, 0.336 mmol) at 0° C. was added HCl/1,4-dioxane (1.68 mL, 4 M, 6.73 mmol). The reaction mixture was warmed to room temperature and stirred for 1 hour. After this time, the mixture was concentrated to dryness in vacuo. The residue was subjected to HPLC (Phenomenex Gemini-NX C18 column, 3 μm, 75×30 mm; 26-56% MeCN/H₂O with 0.05% NH₃·H₂O and 10 mM NH₄HCO₃) to give, after lyophilization, (R)-3-(3-(2-(1H-pyrazolo[3,4-b]pyridin-3-yl)thiazol-4-yl)phenyl)-3-hydroxy-1-methylpyrrolidin-2-one as a colorless solid (68.2 mg, 51%). LC-MS (ESI): mass calcd. for C₂₀H₁₇N₅O₂S 391.11 m/z, found 392.1 [M+H]⁺. ¹H NMR (400 MHz, DMSO-d₆) δ 14.22 (br s, 1H), 8.87-8.82 (m, 1H), 8.70-8.66 (m, 1H), 8.19 (s, 1H), 8.16-8.12 (m, 1H), 8.06-8.01 (m, 1H), 7.52-7.42 (m, 2H), 7.40-7.35 (m, 1H), 6.18 (s, 1H), 3.53-3.46 (m, 1H), 3.44-3.40 (m, 1H), 2.90 (s, 3H), 2.45-2.36 (m, 1H), 2.35-2.26 (m, 1H).

### Example 101. (5R,7R)-7-(3-(2-(1H-Pyrrolo[2,3-b]pyridin-3-yl)thiazol-4-yl)phenyl)-6,7-dihydro-5H-cyclopenta[b]pyridine-5,7-diol

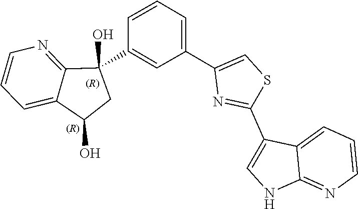

The two cis-alcohol diastereomers of cis-7-(3-(2-(1H-pyrrolo[2,3-b]pyridin-3-yl)thiazol-4-yl)phenyl)-6,7-dihydro-5H-cyclopenta[b]pyridine-5,7-diol (Intermediate 166-B, 68 mg) were separated by SFC over a DAICEL CHIRALPAK AD 250 mm×30 mm×10 μm column. An isocratic elution was used throughout the purification with IPA (containing 0.1% of 25% aq. NH₃): supercritical CO₂=55%: 45% to afford two products: The first eluting diastereomer consisted of the title compound (30.7 mg, 45%) and was obtained as a white solid; LCMS (ESI): mass calcd. for C₂₄H₁₈N₄O₂S 426.11 m/z, found 427.0 [M+H]⁺. ¹H NMR (400 MHz, DMSO-d₆) δ 12.81-11.59 (m, 1H), 8.60-8.53 (m, 1H), 8.47 (dd, J=1.4, 7.8 Hz, 1H), 8.37 (dd, J=1.3, 4.6 Hz, 1H), 8.26 (s, 1H), 7.94-7.83 (m, 4H), 7.44-7.31 (m, 4H), 6.13 (br s, 1H), 5.79 (br s, 1H), 5.16-5.07 (m, 1H), 3.04-2.93 (m, 1H), 2.35-2.28 (m, 1H).

### Example 102. (5S,7S)-7-(3-(2-(1H-Pyrrolo[2,3b]-pyridin-3-yl)thiazol-4-yl)phenyl)-6,7-dihydro-5H-cyclopenta[b]pyridine-5,7-diol

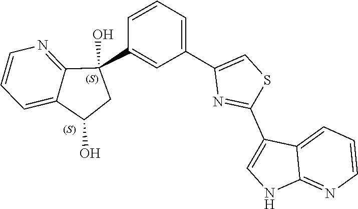

The chiral separation from Example 101 also provided the second eluting diastereomer: (5S,7S)-7-(3-(2-(1H-pyrrolo[2,3-b]pyridin-3-yl)thiazol-4-yl)phenyl)-6,7-dihydro-5H-cyclopenta[b]pyridine-5,7-diol (30.8 mg, 45%) as a white solid. LCMS (ESI), mass calcd. for C₂₄H₁₈N₄O₂S 426.11 m/z, found 427.0 [M+H]⁺. ¹H NMR (400 MHz, DMSO-d₆) δ 12.25 (br s, 1H), 8.56 (dd, J=1.0, 4.8 Hz, 1H), 8.48 (dd, J=1.5, 7.8 Hz, 1H), 8.38 (dd, J=1.6, 4.6 Hz, 1H), 8.25 (s, 1H), 7.92-7.84 (m, 4H), 7.44-7.31 (m, 4H), 6.13 (br s, 1H), 5.79 (br s, 1H), 5.16-5.06 (m, 1H), 3.03-2.95 (m, 1H), 2.35-2.29 (m, 1H).

### Example 103. (5S,7R)-7-(3-(2-(1H-Pyrrolo[2,3-b]pyridin-3-yl)thiazol-4-yl)phenyl)-6,7-dihydro-5H-cyclopenta[b]pyridine-5,7-diol

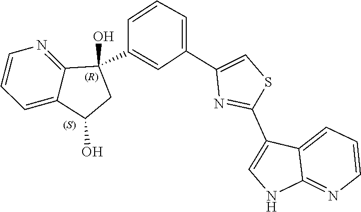

The two trans-alcohol diastereomers of trans-7-(3-(2-(1H-pyrrolo[2,3-b]pyridin-3-yl)thiazol-4-yl)phenyl)-6,7-dihydro-5H-cyclopenta[b]pyridine-5,7-diol (Intermediate 166-A, 33.8 mg) were separated by SFC over a DAICEL CHIRALPAK IG 250 mm×30 mm×10 μm column. An isocratic elution was used throughout the purification which consisted of EtOH (containing 0.1% of 25% aq. NH₃): supercritical CO₂=55%: 45% to give two products. The first eluting diastereomer afforded the title compound (7.2 mg, 21%) as a white solid; LCMS (ESI): mass calcd. for C₂₄H₁₈N₄O₂S 426.11 m/z, found 427.1 [M+H]⁺. ¹H NMR (400 MHz, DMSO-d₆) δ 12.27 (br s, 1H), 8.57 (dd, J=1.5, 8.0 Hz, 1H), 8.48-8.43 (m, 1H), 8.35 (dd, J=1.5, 4.8 Hz, 1H), 8.25 (s, 1H), 8.17-8.13 (m, 1H), 7.93-7.85 (m, 3H), 7.46-7.35 (m, 3H), 7.29-7.24 (m, 1H), 5.90 (s, 1H), 5.71-5.65 (m, 1H), 5.40-5.32 (m, 1H), 2.84-2.78 (m, 1H), 2.32-2.23 (m, 1H).

### Example 104. (5R,7S)-7-(3-(2-(1H-Pyrrolo[2,3-b]pyridin-3-yl)thiazol-4-yl)phenyl)-6,7-dihydro-5H-cyclopenta[b]pyridine-5,7-diol

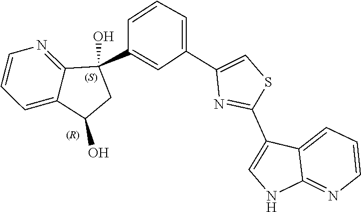

The chiral separation from Example 103 also provided the second eluting diastereomer as the title compound (5.3 mg, 16%) as a white solid. LCMS (ESI): mass calcd. for C₂₄H₁₈N₄O₂S 426.11 m/z, found 427.1 [M+H]⁺. ¹H NMR (400 MHz, DMSO-d₆) δ 12.74-11.71 (m, 1H), 8.58 (dd, J=1.5, 7.9 Hz, 1H), 8.49-8.43 (m, 1H), 8.36 (dd, J=1.5, 4.6 Hz, 1H), 8.25 (s, 1H), 8.17-8.12 (m, 1H), 7.94-7.84 (m, 3H), 7.47-7.35 (m, 3H), 7.30-7.24 (m, 1H), 5.94 (br s, 1H), 5.74 (br s, 1H), 5.43-5.31 (m, 1H), 2.87-2.76 (m, 1H), 2.32-2.23 (m, 1H).

### Example 105. (5S,7S)-7-(3-(2-(1H-Pyrrolo[2,3-b]pyridin-3-yl)thiazol-4-yl)phenyl)-5-methyl-6,7-dihydro-5H-cyclopenta[b]pyridine-5,7-diol

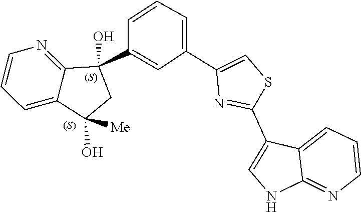

7-(3-(2-(1H-pyrrolo[2,3-b]pyridin-3-yl)thiazol-4-yl)phenyl)-5-methyl-6,7-dihydro-5H-cyclopenta[b]pyridine-5,7-diol (Intermediate 168, 60 mg, 0.136 mmol) was purified by SFC over a DAICEL CHIRALCEL OD 250 mm×30 mm×10 μm column. An isocratic elution was used throughout the purification which consisted of EtOH (containing 0.1% of 25% aq. NH₃): supercritical CO₂=50%: 50% to afford two products: The first eluting diastereomer, (5S,7S)-7-(3-(2-(1H-pyrrolo[2,3-b]pyridin-3-yl)thiazol-4-yl)phenyl)-5-methyl-6,7-dihydro-5H-cyclopenta[b]pyridine-5,7-diol, afforded the title compound (21.6 mg, 35%) as a colorless solid. LCMS (ESI): mass calcd. for C₂₅H₂₀N₄O₂S 440.13 m/z, found 441.0 [M+H]⁺. ¹H NMR (400 MHz, DMSO-d₆) δ 13.05-11.21 (m, 1H), 8.61 (dd, J=1.5, 4.8 Hz, 1H), 8.43 (dd, J=1.6, 7.9 Hz, 1H), 8.37 (dd, J=1.5, 4.7 Hz, 1H), 8.25 (s, 1H), 7.92 (dd, J=1.5, 7.7 Hz, 1H), 7.89-7.81 (m, 3H), 7.47-7.39 (m, 2H), 7.37-7.33 (m, 1H), 7.31-7.26 (m, 1H), 5.94 (br s, 1H), 5.38 (br s, 1H), 2.76-2.59 (m, 2H), 1.41 (s, 3H). The second eluting isomer was (5R,7R)-7-(3-(2-(1H-pyrrolo[2,3-b]pyridin-3-yl)thiazol-4-yl)phenyl)-5-methyl-6,7-dihydro-5H-cyclopenta[b]pyridine-5,7-diol (Example 106).

### Example 106. (5R,7R)-7-(3-(2-(1H-Pyrrolo[2,3-b]pyridin-3-yl)thiazol-4-yl)phenyl)-5-methyl-6,7-dihydro-5H-cyclopenta[b]pyridine-5,7-diol

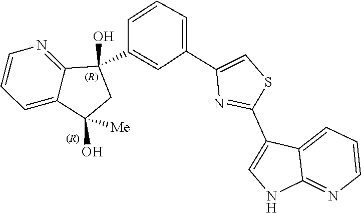

The chiral separation from Example 105 also provided the title compound as the second eluting diastereomer (18.9 mg, 31%) as a colorless solid. LCMS (ESI): mass calcd. for C₂₅H₂₀N₄O₂S 440.13 m/z, found 441.0 [M+H]⁺. ¹H NMR (400 MHz, DMSO-d₆) 512.27 (br s, 1H), 8.60 (dd, J=1.4, 4.6 Hz, 1H), 8.43 (dd, J=1.4, 7.9 Hz, 1H), 8.37 (dd, J=1.4, 4.6 Hz, 1H), 8.25 (s, 1H), 7.92 (dd, J=1.4, 7.6 Hz, 1H), 7.89-7.82 (m, 3H), 7.47-7.39 (m, 2H), 7.37-7.33 (m, 1H), 7.32-7.26 (m, 1H), 5.93 (s, 1H), 5.37 (s, 1H), 2.76-2.60 (m, 2H), 1.41 (s, 3H).

### Example 107. (5R,7S)-7-(3-(2-(1H-Pyrrolo[2,3-b]pyridin-3-yl)thiazol-4-yl)phenyl)-5-methyl-6,7-dihydro-5H-cyclopenta[b]pyridine-5,7-diol

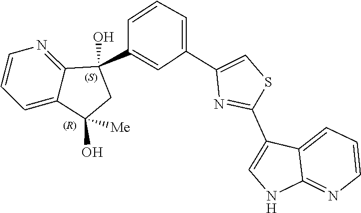

7-(3-(2-(1H-Pyrrolo[2,3-b]pyridin-3-yl)thiazol-4-yl)phenyl)-5-methyl-6,7-dihydro-5H-cyclopenta[b]pyridine-5,7-diol (Intermediate 168, 35 mg, 0.079 mmol) was purified by SFC over a DAICEL CHIRALPAK AD 250 mm×30 mm×10 μm column. An isocratic elution was used throughout the purification which consisted of IPA (containing 0.1% of 25% aq. NH₃): supercritical CO₂=55%: 45% to afford two diastereomers: the first eluting diastereomer afforded the title compound (13.2 mg, 38%) as a colorless solid. LCMS (ESI): mass calcd. for C₂₅H₂₀N₄O₂S 440.13 m/z, found 441.1 [M+H]. ¹H NMR (400 MHz, DMSO-d₆) δ 12.26 (br s, 1H), 8.59 (d, J=8.1 Hz, 1H), 8.48 (d, J=4.6 Hz, 1H), 8.36 (d, J=4.6 Hz, 1H), 8.25 (s, 1H), 8.17 (s, 1H), 7.93-7.82 (m, 3H), 7.45-7.35 (m, 3H), 7.32-7.24 (m, 1H), 5.89 (s, 1H), 5.45 (s, 1H), 2.62-2.57 (m, 2H), 1.65 (s, 3H).

### Example 108. (5S,7R)-7-(3-(2-(1H-Pyrrolo[2,3-b]pyridin-3-yl)thiazol-4-yl)phenyl)-5-methyl-6,7-dihydro-5H-cyclopenta[b]pyridine-5,7-diol

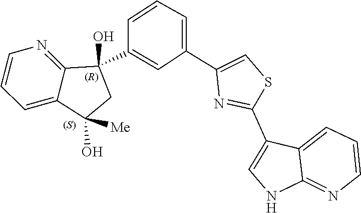

The chiral separation from Example 49 also provided the title compound as the second eluting diastereomer (10.5 mg, 30%) as a colorless solid. LCMS (ESI): mass calcd. for C₂₅H₂₀N₄O₂S 440.13 m/z, found 441.1 [M+H]⁺. ¹H NMR (400 MHz, DMSO-d₆) δ 12.27 (br s, 1H), 8.59 (dd, J=1.5, 7.8 Hz, 1H), 8.47 (dd, J=1.6, 4.8 Hz, 1H), 8.36 (dd, J=1.5, 4.6 Hz, 1H), 8.25 (s, 1H), 8.17 (s, 1H), 7.92-7.81 (m, 3H), 7.46-7.34 (m, 3H), 7.31-7.23 (m, 1H), 5.88 (s, 1H), 5.43 (s, 1H), 2.64-2.56 (m, 2H), 1.64 (s, 3H).

### Example 109. (R)-3-(3-(4-(1H-Pyrrolo[2,3-b]pyridin-3-yl)-2H-1,2,3-triazol-2-yl)phenyl)-3-hydroxy-1-methylpyrrolidin-2-one

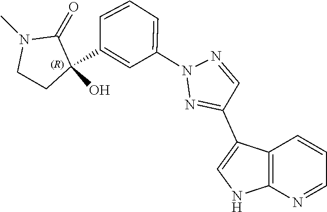

Step A. Sodium hydroxide (1.75 mL, 2 M in water, 3.50 mmol) was added to a mixture of 3-hydroxy-1-methyl-3-(3-(4-(1-tosyl-1H-pyrrolo[2,3-b]pyridin-3-yl)-2H-1,2,3-triazol-2-yl)phenyl)pyrrolidin-2-one (308 mg, 0.583 mmol) and 1,4-dioxane (3 mL). The resultant mixture was heated at 60° C. for 2 h. After this time, the mixture was cooled to room temperature and concentrated to dryness under reduced pressure to afford the product which was purified by FCC (eluent: DCM:MeOH=1:0 to 10:1) and prep. HPLC over a Boston Prime C18 150×30 mm×5 μm column (eluent: 25% to 55% (v/v) ACN and H₂O with 0.05% NH₃·H₂O) to give the title compound (168 mg, 77%) as a white solid. LC-MS (ESI): mass calcd. for C₂₀H₁₈N₆O₂ 374.15 m/z found 375.1 [M+H]⁺.

Step B. Racemic 3-(3-(4-(1H-Pyrrolo[2,3-b]pyridin-3-yl)-2H-1,2,3-triazol-2-yl)phenyl)-3-hydroxy-1-methylpyrrolidin-2-one (168 mg, 0.449 mmol) was further purified by SFC over a DAICEL CHIRALPAK AD 250 mm×30 mm×10 μm column (isocratic elution: ETOH (containing 0.1% of 25% aq. NH₃): supercritical CO₂=50%: 50% (v/v)). The first eluting enantiomer was collected and the volatiles were removed under reduced pressure. The product was suspended in water (10 mL), the mixture frozen using dry ice/acetone, and then lyophilized to dryness to afford (R)-3-(3-(4-(1H-pyrrolo[2,3-b]pyridin-3-yl)-2H-1,2,3-triazol-2-yl)phenyl)-3-hydroxy-1-methylpyrrolidin-2-one (76 mg, 38%) as a colorless solid. LC-MS (ESI): mass calcd. for C₂₀H₁₈N₆O₂ 374.15 m/z found 375.1 [M+H]⁺. ¹H NMR (400 MHz, DMSO-d₆) 812.10 (br s, 1H), 8.58 (d, J=7.6 Hz, 1H), 8.54-8.49 (m, 1H), 8.37-8.31 (m, 1H), 8.24-8.15 (m, 2H), 8.03 (d, J=7.4 Hz, 1H), 7.60-7.50 (m, 1H), 7.37-7.30 (m, 1H), 7.26 (dd, J=4.7, 7.8 Hz, 1H), 6.28 (s, 1H), 3.56-3.45 (m, 2H), 2.88 (s, 3H), 2.44-2.25 (m, 2H).

The chiral separation from Example 52, Step B also provided the second eluting enantiomer, (S)-3-(3-(4-(1H-pyrrolo[2,3-b]pyridin-3-yl)-2H-1,2,3-triazol-2-yl)phenyl)-3-hydroxy-1-methylpyrrolidin-2-one (82 mg, 46%) as a colorless solid. LC-MS (ESI): mass calcd. for C₂₀H₁₈N₆O₂ 374.15 m/z found 375.1 [M+H]⁺. ¹H NMR (400 MHz, DMSO-d₆) δ 12.10 (br s, 1H), 8.61-8.56 (m, 1H), 8.54-8.50 (m, 1H), 8.36-8.32 (m, 1H), 8.23-8.17 (m, 2H), 8.06-8.01 (m, 1H), 7.59-7.52 (m, 1H), 7.36-7.30 (m, 1H), 7.26 (dd, J=4.6, 7.9 Hz, 1H), 6.28 (s, 1H), 3.53-3.45 (m, 1H), 3.44-3.40 (m, 1H), 2.88 (s, 3H), 2.43-2.26 (m, 2H).

### Example 110. (R)-3-(3-(2-(2,5-Difluoro-1H-pyrrolo[2,3-b]pyridin-3-yl)thiazol-4-yl)phenyl)-3-hydroxy-1-methylpyrrolidin-2-one

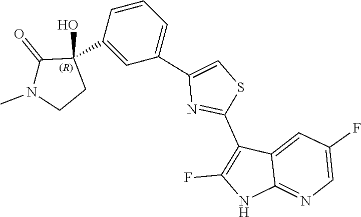

Sodium hydroxide (0.41 mL, 0.82 mmol, 2.0 M in H₂O) was added to a mixture of (R)-3-(3-(2-(2,5-difluoro-1-tosyl-1H-pyrrolo[2,3-b]pyridin-3-yl)thiazol-4-yl)phenyl)-3-hydroxy-1-methylpyrrolidin-2-one (Intermediate 169, 320 mg, 0.138 mmol, purity: 25%) and 1,4-dioxane (2 mL). The resultant mixture was heated at 60° C. for 16 hours. After this time, the mixture was quenched with water (5 mL) and extracted with ethyl acetate (5 mL×3). The combined organic extracts was dried over anhydrous Na₂SO₄, filtered, and concentrated to dryness under reduced pressure to afford the product, which was purified by FCC (eluent: petroleum ether:ethyl acetate=1:0 to 0:1), and then purified by prep-HPLC over a Phenomenex Gemini-NX C18 75×30 mm×3 μm column (eluent: 15% to 45% (v/v) ACN and water (0.05% NH₃H₂O+10 mM NH₄HCO₃)), and finally purified by SFC over a DAICEL CHIRALCEL OD-H 250 mm×30 mm×5 μm column (isocratic elution: 0.1% NH₃H₂O EtOH: supercritical CO₂=35%: 65%) to afford the title compound (7.6 mg, 13%) as a yellow solid. LCMS (ESI): mass calcd. for C₂₁H₁₆F₂N₄O₂S 426.10 m/z, found 427.0 [M+1]⁺. ¹H NMR (400 MHz, DMSO-d₆) δ8.45-8.38 (m, 1H), 8.35-8.29 (m, 1H), 8.11-8.02 (m, 2H), 8.00-7.93 (m, 1H), 7.50-7.44 (m, 1H), 7.41-7.35 (m, 1H), 6.15 (s, 1H), 3.47-3.45 (m, 2H), 2.90 (s, 3H), 2.39-2.28 (m, 2H).

### Example 111. (R)-3-(3-(2-(1H-Pyrrolo[3,2-b]pyridin-1-yl)thiazol-4-yl)phenyl)-3-hydroxy-1-methylpyrrolidin-2-one

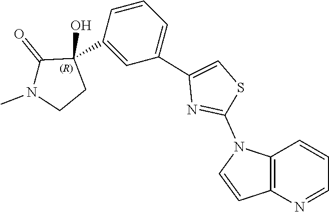

To a microwave vial containing 4-bromo-2-(1H-pyrrolo[3,2-b]pyridin-1-yl)thiazole (220 mg, 0.785 mmol), (R,S)-3-hydroxy-1-methyl-3-(3-(4,4,5,5-tetramethyl-1,3,2-dioxaborolan-2-yl)phenyl)pyrrolidin-2-one (Intermediate 4, 300 mg, 0.946 mmol) and K₃PO₄ (322 mg, 1.52 mmol) were added 1,4-dioxane/H₂O (4:1) (10 mL). The resultant mixture was sparged with N₂ for 5 minutes and then treated with Xphos-Pd-G₂ (123 mg, 0.156 mmol). The resultant mixture was sparged with N₂ for another 5 minutes and then heated at 100° C. via microwave irradiation for 1 hours. After this time, the mixture was cooled to room temperature and filtered. The filtrate was diluted with H₂O (20 mL) and extracted with ethyl acetate (20 mL×3). The combined organic extracts were washed with brine (8 mL), dried over anhydrous Na₂SO₄, filtered, and concentrated under reduced pressure to give the product, which was purified by preparative HPLC with a Welch Xtimate C18 150×25 mm×5 μm column, (eluent: 30% to 50% (v/v) CH₃CN and H₂O with 0.04% NH₃·H₂O+10 mM NH₄HCO₃) to afford the pure product. The product was suspended in water (10 mL) and MeCN (2 mL), the mixture was frozen using dry ice/ethanol and then lyophilized to dryness to afford the racemic product (150 mg). The racemic product was further separated by SFC over DAICEL CHIRALPAK AD 250 mm×30 mm, 10 μm column (eluent: 55% to 55% (v/v) supercritical CO₂ in EtOH and H₂O with 0.1% NH₃). The first eluting enantiomer was collected, and the volatiles were removed under reduced pressure. The product was suspended in water (5 mL), the mixture frozen using dry ice/acetone, and then lyophilized to dryness to afford (R)-3-(3-(2-(1H pyrrolo[3,2-b]pyridin-1-yl)thiazol-4-yl)phenyl)-3-hydroxy-1-methylpyrrolidin-2-one (46 mg, 15%) as a colorless solid. LCMS (ESI): mass calcd. for C₂₁H₁₈N₄O₂S 390.12 m/z, found 391.2 [M+1]⁺. ¹H NMR (400 MHz, DMSO-d₆): 8.81 (d, J=8.2 Hz, 1H), 8.56 (dd, J=1.3, 4.6 Hz, 1H), 8.30 (d, J=3.7 Hz, 1H), 8.04 (s, 1H), 7.98-7.90 (m, 2H), 7.52-7.34 (m, 3H), 6.99 (d, J=3.7 Hz, 1H), 6.19 (br s, 1H), 3.53-3.37 (m, 2H), 2.89 (s, 3H), 2.43-2.24 (m, 2H). The second eluting enantiomer was collected, and the solvents were removed under reduced pressure. This product was suspended in water (5 mL), the mixture frozen using dry ice/acetone, and then lyophilized to dryness to afford (S)-3-(3-(2-(1H-pyrrolo[3,2-b]pyridin-1-yl)thiazol-4-yl)phenyl)-3-hydroxy-1-methylpyrrolidin-2-one (48.5 mg, 16%) as a colorless solid. LCMS (ESI): mass calcd. for C₂₁H₁₆N₄O₂S 390.12 m/z, found 391.2 [M+1]+. ¹H NMR (400 MHz, DMSO-d₆): 8.81 (d, J=8.2 Hz, 1H), 8.56 (dd, J=1.1, 4.6 Hz, 1H), 8.29 (d, J=3.5 Hz, 1H), 8.04 (s, 1H), 7.98-7.91 (m, 2H), 7.51-7.34 (m, 3H), 6.99 (d, J=3.3 Hz, 1H), 6.19 (br s, 1H), 3.53-3.37 (m, 2H), 2.89 (s, 3H), 2.43-2.25 (m, 2H).

### Example 112. (R)-3-(3-(2-(5-Amino-1H-pyrrolo[3,2-b]pyridin-1-yl)thiazol-4-yl)phenyl)-3-hydroxy-1-methylpyrrolidin-2-one

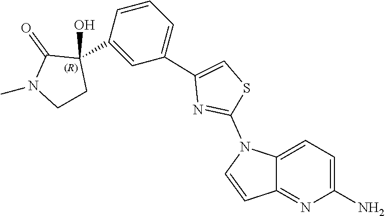

1H-Pyrrolo[3,2-b]pyridin-5-amine (377 mg, 2.83 mmol) was added to a mixture of (R)-3-(3-(2-bromothiazol-4-yl)phenyl)-3-hydroxy-1-methylpyrrolidin-2-one (Intermediate 187, 200 mg, 0.566 mmol), Cs₂CO₃ (369 mg, 1.13 mmol), and DMF (5 mL). The resultant mixture was sparged with N₂ for 5 minutes and then treated with CuI (54 mg, 0.28 mmol) and 1,10-phenanthroline (50 mg, 0.28 mmol). The resultant mixture was sparged with N₂ for another 5 minutes and then stirred at 100° C. for 16 hours. After this time, the mixture was cooled to room temperature, poured into H₂O (10 mL), and extracted with ethyl acetate (20 mL×3). The combined organic extracts were washed with brine, dried over anhydrous Na₂SO₄, filtered, and concentrated to dryness under reduced pressure to give the product, which was purified by prep-HPLC using a Phenomenex Gemini-NX 150×30 mm×5 μm column (eluent: 53% to 23% (v/v) water (0.05% NH₃H₂O)-ACN) to afford pure product. The product was suspended in water (5 mL), the mixture was frozen using dry ice/ethanol, and then lyophilized to dryness to afford the compound (8.4 mg, 4%) as a colorless solid. LCMS (ESI): mass calcd. for C₂₁H₁₉N₅O₂S 405.13 m/z, found 406.0 [M+H]⁺. ¹H NMR (400 MHz, MeOD-d₄) δ 8.59-8.52 (m, 1H), 8.07 (s, 1H), 7.93-7.86 (m, 1H), 7.81-7.75 (m, 1H), 7.52 (s, 1H), 7.47-7.36 (m, 2H), 6.67-6.60 (m, 1H), 6.55-6.51 (m, 1H), 3.60-3.51 (m, 1H), 3.50-3.41 (m, 1H), 2.99 (s, 3H), 2.55-2.35 (m, 2H).

### Example 113. (R)-3-Hydroxy-1-methyl-3-(3-(2-(5-methyl-1H-pyrrolo[3,2-b]pyridin-3-yl)thiazol-4-yl)phenyl)pyrrolidin-2-one

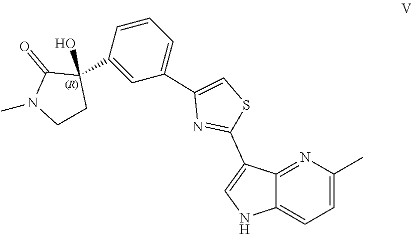

Sodium hydroxide (2.4 mL, 2.0 M in H₂O, 4.8 mmol) was added to a solution of (R)-3-hydroxy-1-methyl-3-(3-(2-(5-methyl-1-tosyl-1H-pyrrolo[3,2-b]pyridin-3-yl)thiazol-4-yl)phenyl)pyrrolidin-2-one (Intermediate 185, 435 mg, 0.779 mmol) and 1,4-dioxane (20 mL). The resultant mixture was heated at 60° C. for 3 hours. After this time, the mixture was cooled to room temperature and concentrated under reduced pressure to afford the product, which was purified by preparative HPLC using a Boston Prime C18 150×30 mm×5 μm column (eluent: 32% to 62% (v/v) CH₃CN and H₂O with 10 mM NH₄HCO₃) to give pure product. The product was suspended in water (10 mL), the mixture frozen using dry ice/acetone, and then lyophilized to dryness to afford the title compound (73.8 mg, 23%) as a colorless solid. LCMS (ESI): mass calcd. for C₂₂H₂₀N₄O₂S 404.13 m/z, found 405.0 [M+H]⁺. ¹H NMR (400 MHz, DMSO-d₆) δ 11.80 (br s, 1H), 8.33 (s, 1H), 8.08 (s, 1H), 7.98-7.92 (m, 2H), 7.83-7.78 (m, 1H), 7.46-7.39 (m, 1H), 7.34-7.28 (m, 1H), 7.16-7.11 (m, 1H), 6.11 (br s, 1H), 3.51-3.42 (m, 2H), 2.88 (s, 3H), 2.65 (s, 3H), 2.44-2.35 (m, 1H), 2.34-2.22 (m, 1H).

### Example 114. (R)-3-(3-(5-(1H-Pyrrolo[2,3-b]pyridin-3-yl)furan-2-yl)phenyl)-3-hydroxy-1-methylpyrrolidin-2-one

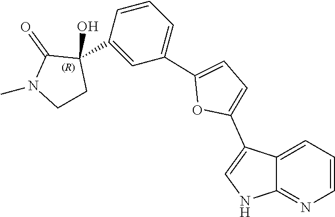

Aqueous NaOH (0.5 mL, 2.0 M, 1.0 mmol) was added to a solution of (R)-3-hydroxy-1-methyl-3-(3-(5-(1-(phenylsulfonyl)-1H-pyrrolo[2,3-b]pyridin-3-yl)furan-2-yl)phenyl)pyrrolidin-2-one (Intermediate 171, 250 mg, 0.487 mmol) and 1,4-dioxane (3 mL). The resultant mixture was heated at 70° C. for 1 hour. After this time, the mixture was cooled to room temperature and concentrated under reduced pressure to afford the product, which was purified by preparative HPLC using a Boston Prime C18 150 mm×30 mm×5 μm column (eluent: 35% to 65% (v/v) CH₃CN and H₂O with 0.05% NH₃H₂O+10 mM NH₄HCO₃) to afford pure product. The product was suspended in water (10 mL), the mixture frozen using dry ice/EtOH, and then lyophilized to dryness to afford the title compound (59.7 mg, 32%) as a light yellow solid. LC-MS (ESI): mass calcd. For C₂₂H₁₉N₃O₃ 373.14 m/z found 374.0 [M+H]⁺. ¹H NMR (400 MHz, DMSO-d₆) S 12.06 (br s, 1H), 8.41 (dd, J=1.5, 7.8 Hz, 1H), 8.35-8.30 (m, 1H), 7.99 (s, 1H), 7.80 (s, 1H), 7.72 (d, J=7.8 Hz, 1H), 7.45-7.38 (m, 1H), 7.28-7.20 (m, 2H), 7.05 (d, J=3.5 Hz, 1H), 6.86 (d, J=3.5 Hz, 1H), 6.13 (s, 1H), 3.52-3.38 (m, 2H), 2.88 (s, 3H), 2.43-2.24 (m, 2H).

### Example 115. (R)-3-(3-(4-(1H-Pyrrolo[2,3-b]pyridin-3-yl)furan-2-yl)phenyl)-3-hydroxy-1-methylpyrrolidin-2-one

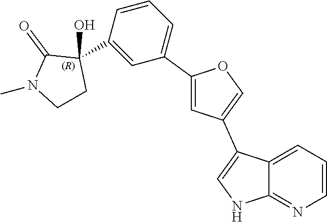

Sodium hydroxide (1.2 mL, 2.0 M in H₂O, 2.4 mmol) was added to a solution of (R)-3-hydroxy-1-methyl-3-(3-(4-(1-(phenylsulfonyl)-1H-pyrrolo[2,3-b]pyridin-3-yl)furan-2-yl)phenyl)pyrrolidin-2-one (Intermediate 172, 200 mg, 0.389 mmol) and 1,4-dioxane (6 mL). The resultant mixture was heated at 60° C. for 1 hour. After this time, the mixture was cooled to room temperature and concentrated to dryness under reduced pressure to afford the product, which was purified by preparative HPLC using a Phenomenex Gemini NX-C18 75×30 mm×3 μm column (eluent: 28% to 58% (v/v) CH₃CN and H₂O with 0.05% NH₃H₂O+10 mM NH₄HCO₃) to afford pure product. The product was suspended in water (10 mL), the mixture frozen using dry ice/acetone, and then lyophilized to dryness to afford the title compound (16.5 mg, 11%) as a white solid. LCMS (ESI): mass calcd. for C₂₂H₁₉N₃O₃ 373.14 m/z, found 374.0 [M+H]⁺. ¹H NMR (400 MHz, DMSO-d₆) δ 11.80 (br s, 1H), 8.32-8.25 (m, 3H), 7.88 (s, 1H), 7.82-7.77 (m, 1H), 7.70-7.63 (m, 1H), 7.46-7.38 (m, 2H), 7.31-7.24 (m, 1H), 7.19-7.13 (m, 1H), 6.12 (s, 1H), 3.52-3.36 (m, 2H), 2.86 (s, 3H), 2.40-2.22 (m, 2H).

### Example 116. (R)-3-(3-(2-(1H-Pyrrolo[2,3-b]pyridin-3-yl)oxazol-4-yl)phenyl)-3-hydroxy-1-methylpyrrolidin-2-one

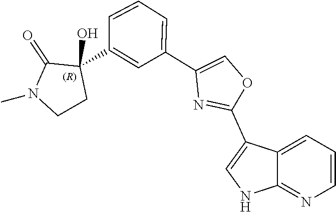

Sodium hydroxide (2.16 mL, 2.0 M in water, 4.31 mmol) was added to a mixture of (R)-3-hydroxy-1-methyl-3-(3-(2-(1-tosyl-1H-pyrrolo[2,3-b]pyridin-3-yl)oxazol-4-yl)phenyl)pyrrolidin-2-one (Intermediate 173, 380 mg, 0.719 mmol) and 1,4-dioxane (5 mL). The resultant mixture was heated at 60° C. for 2 hours. After this time, the mixture was cooled to room temperature and concentrated to dryness under reduced pressure to afford the product, which was purified by preparative HPLC over a Boston Prime C18 150×30 mm×5 μm column (eluent: 60% to 85% (v/v) CH₃CN and H₂O with 0.1% TFA) to afford pure product. The product was suspended in water (10 mL), the mixture frozen using dry ice/acetone, and then lyophilized to dryness to afford the title compound (120 mg, 42%) as a white solid. LCMS (ESI): mass calcd. for C₂₁H₁₈N₄O₃ 374.14 m/z, found 375.0 [M+H]⁺. ¹H NMR (400 MHz, DMSO-d₆) δ 12.41 (br s, 1H), 8.62-8.57 (m, 2H), 8.39-8.36 (m, 1H), 8.26 (s, 1H), 7.96-7.93 (m, 1H), 7.82-7.78 (m, 1H), 7.45-7.40 (m, 1H), 7.34-7.27 (m, 2H), 6.12 (br s, 1H), 3.51-3.44 (m, 1H), 3.42-3.37 (m, 1H), 2.88 (s, 3H), 2.41-2.24 (m, 2H).

### Example 117. (R)-3-(3-(5-(1H-Pyrrolo[2,3-b]pyridin-3-yl)-1H-pyrazol-3-yl)phenyl)-3-hydroxy-1-methylpyrrolidin-2-one

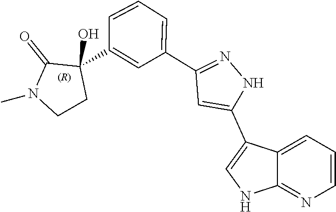

Tetrabutylammonium fluoride (4.37 mL, 1.0 M in THF, 4.37 mmol) was added to a mixture of 3-(3-(5-(1H-pyrrolo[2,3-b]pyridin-3-yl)-1-((2-(trimethylsilyl)ethoxy)methyl)-1H-pyrazol-3-yl)phenyl)-3-hydroxy-1-methylpyrrolidin-2-one (Intermediate 174, 220 mg, 0.437 mmol) and THF (15 mL). The reaction mixture was heated at 90° C. for 16 hours. After this time, the mixture was cooled to room temperature, poured into water (30 mL), and extracted with ethyl acetate (30 mL×3). The combined organic extracts were washed with brine (50 mL), dried over anhydrous Na₂SO₄, filtered, and concentrated under reduced pressure to give the product, which was partially purified by preparative HPLC using a Phenomenex Gemini NX-C18 75×30 mm×3 μm column (eluent: 20% to 50% (v/v) MeCN and water (0.05% NH₃H₂O+10 mM NH₄HCO₃)) to afford a still impure product. The product was further purified by SFC over a DAICEL CHIRALCEL OJ-H 250 mm×30 mm×50 μm column (isocratic elution: EtOH (containing 0.1% of 25% aq. NH₃): supercritical CO₂, 35%: 65% to 35%: 65% (v/v)). The pure fractions were collected, and the volatiles were removed under reduced pressure. The product was suspended in water (10 mL), the mixture frozen using dry ice/acetone, and then lyophilized to dryness to afford title compound (12.1 mg, 7%) as a colorless solid. LCMS (ESI): mass calcd. for C₂₁H₁₉N₅O₂ 373.15 m/z, found 374.0 [M+H]⁺. ¹H NMR (400 MHz, DMSO-d₆) δ 13.28-12.96 (m, 1H), 12.11-11.63 (m, 1H), 8.58-8.35 (m, 1H), 8.34-8.19 (m, 1H), 8.04-7.63 (m, 3H), 7.50-7.25 (m, 2H), 7.23-7.12 (m, 1H), 7.11-6.98 (m, 1H), 6.08 (br s, 1H), 3.51-3.43 (m, 2H), 2.87 (s, 3H), 2.43-2.34 (m, 1H), 2.32-2.23 (m, 1H).

### Example 118. (R)-3-Hydroxy-1-methyl-3-(3-(1-methyl-3-(1H-pyrrolo[2,3-b]pyridin-3-yl)-1H-pyrazol-5-yl)phenyl)pyrrolidin-2-one

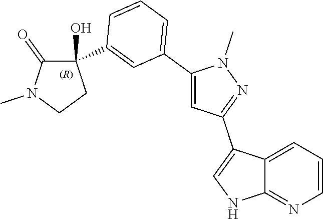

Sodium hydroxide (2.2 mL, 4.4 mmol, 2.0 M in water) was added to a solution of (R)-3-hydroxy-1-methyl-3-(3-(1-methyl-3-(1-(phenylsulfonyl)-1H-pyrrolo[2,3-b]pyridin-3-yl)-1H-pyrazol-5-yl)phenyl)pyrrolidin-2-one (Intermediate 175, 290 mg, 0.550 mmol) and 1,4-dioxane (9 mL). The resultant mixture was heated to 60° C. for 16 hours. After this time, the mixture was cooled to room temperature and concentrated to dryness under reduced pressure to give the product which was purified by prep. HPLC using a Phenomenex Gemini-NX 80×40 mm×3 μm column (eluent: 20% to 40% (v/v) MeCN and water (0.05% NH₃H₂O+10 mM NH₄HCO₃)), and then purified by SFC over DAICEL CHIRALCEL OD-H 250×30 mm×5 μm column (isocratic elution: EtOH (containing 0.1% of 25% aq. NH₃): supercritical CO₂, 40%: 60% to 40%: 60% (v/v)). The pure fractions were combined, and the volatiles were removed under reduced pressure. The product was suspended in water (10 mL), the mixture frozen using dry ice/acetone, and then lyophilized to dryness to afford the title compound (56.1 mg, 26%) as a colorless solid. LCMS (ESI): mass calcd. for C₂₂H₂₁N₅O₂ 387.17 m/z, found 388.1 [M+H]⁺. ¹H NMR (400 MHz, DMSO-d₆) δ 11.77 (br s, 1H), 8.51 (dd, J=1.4, 7.9 Hz, 1H), 8.26 (dd, J=1.6, 4.6 Hz, 1H), 7.91-7.88 (m, 1H), 7.59-7.56 (m, 1H), 7.52-7.48 (m, 2H), 7.46-7.41 (m, 1H), 7.15 (dd, J=4.6, 7.9 Hz, 1H), 6.78 (s, 1H), 6.14 (s, 1H), 3.90 (s, 3H), 3.50-3.42 (m, 1H), 3.41-3.36 (m, 1H), 2.85 (s, 3H), 2.44-2.35 (m, 1H), 2.32-2.23 (m, 1H).

### Example 119. (R)-3-Hydroxy-1-methyl-3-(3-(4-methyl-3-(1H-pyrrolo[2,3-b]pyridin-3-yl)-1H-pyrazol-5-yl)phenyl)pyrrolidin-2-one

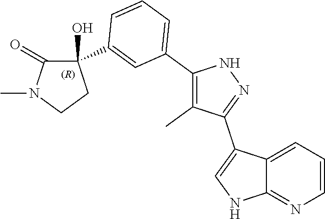

Tetrabutylammonium fluoride (10 mL, 1.0 M in THF, 10 mmol) and (3R)-3-hydroxy-1-methyl-3-(3-(4-methyl-5-(1H-pyrrolo[2,3-b]pyridin-3-yl)-1-((2-(trimethylsilyl)ethoxy)methyl)-1H-pyrazol-3-yl)phenyl)pyrrolidin-2-one (Intermediate 176, Step E, 500 mg, 0.966 mmol) were added to a 50 mL flask. The resultant mixture was heated at 90° C. for 16 hours. As the starting material remained, the mixture was treated with additional tetrabutylammonium fluoride (10 mL, 1.0 M in THF, 10 mmol) and stirred at 90° C. for another 16 hours. After this time, the mixture was concentrated to dryness under reduced pressure to afford the product, which was purified by FCC (eluent: petroleum ether:ethyl acetate=1:0 to 0:1) and prep-HPLC over Xtimate C18 150×40 mm×5 μm column (eluent: 25% to 35% (v/v) MeCN and water (0.05% NH₃·H₂O)) to afford a still impure product. The product was further purified by SFC over DAICEL CHIRALCEL OJ 250 mm×30 mm×10 μm column (isocratic elution: EtOH (containing 0.1% of 25% aq. NH₃): supercritical CO₂, 40%: 60% to 40%: 60% (v/v)). The pure fractions were combined, and the volatiles were removed under reduced pressure. The product was suspended in water (10 mL), the mixture frozen using dry ice/acetone, and then lyophilized to dryness to afford the title compound (20.2 mg, 32%) as a colorless solid. LCMS (ESI): mass calcd. for C₂₂H₂₁N₅O₂ 387.17 m/z, found 388.2 [M+1]⁺. ¹H NMR (400 MHz, DMSO-d₆) δ 13.00-12.82 (m, 1H), 12.13-11.68 (m, 1H), 8.55-7.99 (m, 2H), 7.74-7.70 (m, 1H), 7.64-7.27 (m, 4H), 7.20-7.11 (m, 1H), 6.18-5.96 (m, 1H), 3.51-3.36 (m, 2H), 2.86 (s, 3H), 2.41-2.35 (m, 1H), 2.33-2.26 (m, 3H), 2.25-2.22 (m, 1H).

### Example 120. (R)-3-hydroxy-1-methyl-3-(3-(1-methyl-2-(1H-pyrrolo[2,3-b]pyridin-3-yl)-1H-imidazol-5-yl)phenyl)pyrrolidin-2-one

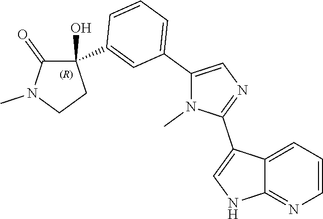

Sodium hydroxide (540 μL, 1.08 mmol, 2.0 M in water) was added to a solution of (R)-3-hydroxy-1-methyl-3-(3-(1-methyl-2-(1-tosyl-1H-pyrrolo[2,3-b]pyridin-3-yl)-1H-imidazol-5-yl)phenyl)pyrrolidin-2-one (Intermediate 177, 146 mg, 0.270 mmol) and 1,4-dioxane (3 mL). The resultant mixture was heated at 60° C. for 2 hours. After this time, the mixture was cooled to room temperature and concentrated to dryness under reduced pressure to give the product, which was purified with prep. HPLC using a Phenomenex Gemini NX-C18, 75×30 mm×3 μm column (eluent: 16% to 46% (v/v) MeCN and water (0.05% NH₃H₂O+10 mM NH₄HCO₃)) to afford the purified product. The product was suspended in water (10 mL), the mixture frozen using dry ice/ethanol, and then lyophilized to dryness to afford the title compound (30 mg, 28%) as a colorless solid. LCMS (ESI): mass calcd. for C₂₂H₂₁N₅O₂ 387.17 m/z, found 388.1 [M+H]⁺. ¹H NMR (400 MHz, DMSO-d₆) δ 12.10 (br s, 1H), 8.57-8.51 (m, 1H), 8.34-8.29 (m, 1H), 8.00-7.95 (m, 1H), 7.52-7.41 (m, 3H), 7.39-7.34 (m, 1H), 7.22-7.14 (m, 2H), 6.12 (s, 1H), 3.76 (s, 3H), 3.49-3.42 (m, 2H), 2.86 (s, 3H), 2.43-2.21 (m, 2H).

### Example 121. (R)-3-(3-(3-(1H-Pyrrolo[2,3-b]pyridin-3-yl)-1H-1,2,4-triazol-1-yl)phenyl)-3-hydroxy-1-methylpyrrolidin-2-one

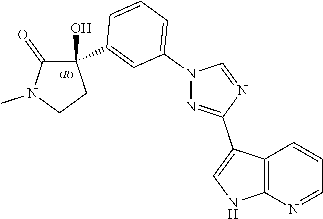

Sodium hydroxide (2.3 mL, 4.6 mmol, 2 M in H₂O) was added to a solution of 3-hydroxy-1-methyl-3-(3-(3-(1-tosyl-1H-pyrrolo[2,3-b]pyridin-3-yl)-1H-1,2,4-triazol-1-I)phenyl)pyrrolidin-2-one (Intermediate 178, 410 mg, 0.776 mmol) and 1,4-dioxane (8 mL). The resultant mixture was heated at 60° C. for 3 hours. After this time, the mixture was cooled to room temperature, diluted with H₂O (10 mL) and extracted with ethyl acetate (20 mL×3). The combined organic extracts were washed with brine, dried over anhydrous Na₂SO₄, filtered, and concentrated to give the product, which was purified by FCC (eluent: methylene chloride:methanol=1:0 to 10:1) to afford (R,S)-3-(3-(3-(1H-pyrrolo[2,3-b]pyridin-3-yl)-1H-1,2,4-triazol-1-yl)phenyl)-3-hydroxy-1-methylpyrrolidin-2-one. The (R) and (S) enantiomers of (R,S)-3-(3-(3-(1H-pyrrolo[2,3-b]pyridin-3-yl)-1H-1,2,4-triazol-1-yl)phenyl)-3-hydroxy-1-methylpyrrolidin-2-one were separated by SFC over DAICEL CHIRALPAK AD, 250 mm×30 mm×10 μm column (isocratic elution: IPA (containing 0.1% of 25% aq. NH₃): supercritical CO₂, 50%: 50% to 50%: 50% (v/v)). The first eluting enantiomer was the title compound, which was further purified by preparative HPLC using a Phenomenex Gemini-NX C18 75 mm×30 mm×3 μm column (eluent: 20% to 50% (v/v) CH₃CN and H₂O with 10 mM NH₄HCO₃) to afford the (R)-3-(3-(3-(1H-pyrrolo[2,3-b]pyridin-3-yl)-1H-1,2,4-triazol-1-yl)phenyl)-3-hydroxy-1-methylpyrrolidin-2-one (51 mg, 17%, R_{T} of SFC=4.719 min) as a colorless solid. LC-MS (ESI): mass calcd. For C₂₀H₁₈N₆O₂ 374.15 m/z found 375.1 [M+H]⁺. ¹H NMR (400 MHz, MeOD-d₄) δ 9.11 (s, 1H), 8.75-8.69 (m, 1H), 8.30-8.25 (m, 1H), 8.06-8.02 (m, 2H), 7.86-7.81 (m, 1H), 7.58-7.52 (m, 1H), 7.45-7.40 (m, 1H), 7.28-7.22 (m, 1H), 3.63-3.46 (m, 2H), 2.98 (s, 3H), 2.58-2.50 (m, 1H), 2.48-2.37 (m, 1H).

The second eluting enantiomer, (S)-3-(3-(3-(1H-pyrrolo[2,3-b]pyridin-3-yl)-1H-1,2,4-triazol-1-yl)phenyl)-3-hydroxy-1-methylpyrrolidin-2-one, (32.8 mg, 11%, R_{T} of SFC=5.717 min) was obtained as a colorless solid. LC-MS (ESI): mass calcd. For C₂₀H₁₈N₆O₂ 374.15 m/z found 375.2 [M+H]⁺. ¹H NMR (400 MHz, MeOD-d₄) δ 9.10 (s, 1H), 8.75-8.68 (m, 1H), 8.31-8.24 (m, 1H), 8.08-8.01 (m, 2H), 7.87-7.81 (m, 1H), 7.59-7.51 (m, 1H), 7.47-7.40 (m, 1H), 7.28-7.22 (m, 1H), 3.63-3.44 (m, 2H), 2.98 (s, 3H), 2.58-2.50 (m, 1H), 2.47-2.38 (m, 1H).

### Example 122. (R)-3-(3-(3-(1H-Pyrrolo[2,3-b]pyridin-3-yl)-1H-pyrazol-1-yl)phenyl)-3-hydroxy-1-methylpyrrolidin-2-one

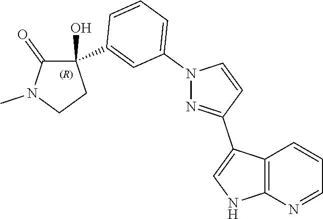

Step A. 3-(3-(3-(1H-Pyrrolo[2,3-b]pyridin-3-yl)-1H-pyrazol-1-yl)phenyl)-3-hydroxy-1-methylpyrrolidin-2-one. Sodium hydroxide (1.71 mL, 2.0 M in water, 3.41 mmol) was added to a mixture of 3-hydroxy-1-methyl-3-(3-(3-(1-tosyl-1H-pyrrolo[2,3-b]pyridin-3-yl)-1H-pyrazol-1-yl)phenyl)pyrrolidin-2-one (Intermediate 179, 300 mg, 0.569 mmol) and 1,4 dioxane (3 mL). The resultant mixture was heated at 60° C. for 16 hours. After this time, the mixture was cooled to room temperature and concentrated to dryness under reduced pressure to afford the racemic product, which was purified with prep. HPLC over Boston Prime C18 150×30 mm×5 μm (eluent: 27% to 55% (v/v) ACN and H₂O with 0.05% NH₃·H₂O). The pure fractions were combined, and the solvents were removed under vacuum. The residue was re-suspended in water (20 mL) and the resulting mixture was lyophilized to dryness to remove the solvent residue completely to give title compound (121 mg, 52%) as a white solid. LC-MS (ESI): mass calcd. For C₂₁H₁₉N₅O₂ 373.15 m/z found 374.1 [M+H]⁺. ¹H NMR (400 MHz, CDCl₃) δ 10.46 (br s, 1H), 8.60-8.54 (m, 1H), 8.36-8.31 (m, 1H), 7.74-7.67 (m, 1H), 7.61-7.58 (m, 1H), 7.56 (d, J=2.6 Hz, 1H), 7.44-7.36 (m, 2H), 7.19-7.12 (m, 2H), 6.51 (d, J=2.6 Hz, 1H), 5.98 (br s, 1H), 3.67-3.58 (m, 1H), 3.49-3.44 (m, 1H), 3.10 (s, 3H), 2.60-2.51 (m, 1H), 2.50-2.42 (m, 1H).

Step B. (R)-3-(3-(3-(1H-pyrrolo[2,3-b]pyridin-3-yl)-1H-pyrazol-1-yl)phenyl)-3-hydroxy-1-methylpyrrolidin-2-one. The (R) and (S)-enantiomers of (R,S)-3-(3-(3-(1H-pyrrolo[2,3-b]pyridin-3-yl)-1H-pyrazol-1-yl)phenyl)-3-hydroxy-1-methylpyrrolidin-2-one (121 mg, 0.324 mmol) were separated by SFC over DAICEL CHIRALPAK AS 250 mm×30 mm×10 μm column (isocratic elution: EtOH (containing 0.1% of 25% aq. NH₃): supercritical CO₂, 50%: 50% to 50%: 50% (v/v)). The first eluting enantiomer from SFC separation provided the title compound, (R)-3-(3-(3-(1H-pyrrolo[2,3-b]pyridin-3-yl)-1H-pyrazol-1-yl)phenyl)-3-hydroxy-1-methylpyrrolidin-2-one, (46.2 mg, 38%, R_{T} of SFC=3.827 min) as a colorless solid. LC-MS (ESI): mass calcd. for C₂₁H₁₉N₅O₂ 373.15 m/z, found 374.1 [M+H]⁺. ¹H NMR (400 MHz, DMSO-d₆) δ 11.88 (br s, 1H), 8.63-8.58 (m, 1H), 8.50 (d, J=2.4 Hz, 1H), 8.30-8.27 (m, 1H), 8.04-8.00 (m, 1H), 7.97-7.93 (m, 1H), 7.84-7.78 (m, 1H), 7.50-7.44 (m, 1H), 7.26-7.17 (m, 2H), 6.95 (d, J=2.4 Hz, 1H), 6.21 (s, 1H), 3.51-3.49 (m, 2H), 2.88 (s, 3H), 2.43-2.35 (m, 1H), 2.34-2.25 (m, 1H). The second eluting enantiomer, (S)-3-(3-(3-(1H-pyrrolo[2,3-b]pyridin-3-yl)-1H-pyrazol-1-yl)phenyl)-3-hydroxy-1-methylpyrrolidin-2-one, (45.6 mg, 38%, R_{T} of SFC=4.908 min) was obtained as a colorless solid. LC-MS (ESI): mass calcd. for C₂₁H₁₉N₅O₂ 373.15 m/z, found 374.1 [M+H]⁺. ¹H NMR (400 MHz, DMSO-d₆) δ 11.88 (br s, 1H), 8.64-8.58 (m, 1H), 8.51 (d, J=2.7 Hz, 1H), 8.31-8.26 (m, 1H), 8.04-8.00 (m, 1H), 7.97-7.93 (m, 1H), 7.84-7.78 (m, 1H), 7.51-7.44 (m, 1H), 7.27-7.17 (m, 2H), 6.95 (d, J=2.4 Hz, 1H), 6.21 (s, 1H), 3.50-3.46 (m, 2H), 2.88 (s, 3H), 2.43-2.35 (m, 1H), 2.34-2.25 (m, 1H).

### Example 123. (R)-3-(3-(4-(1H-Pyrrolo[2,3-b]pyridin-3-yl)-1H-1,2,3-triazol-1-yl)phenyl)-3-hydroxy-1-methylpyrrolidin-2-one

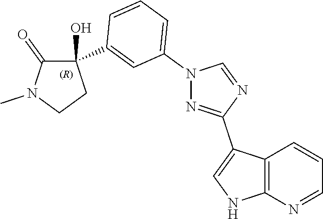

Step A. (R,S)-3-(3-(4-(1H-pyrrolo[2,3-b]pyridin-3-yl)-1H-1,2,3-triazol-1-yl)phenyl)-3-hydroxy-1-methylpyrrolidin-2-one. Sodium hydroxide (0.74 mL, 2.0 M in water, 1.5 mmol) was added to a mixture of 3-hydroxy-1-methyl-3-(3-(4-(1-tosyl-1H-pyrrolo[2,3-b]pyridin-3-yl)-1H-1,2,3-triazol-1-yl)phenyl)pyrrolidin-2-one (Intermediate 180, 130 mg, 0.246 mmol) and 1,4-dioxane (1 mL). The resultant mixture was heated at 60° C. for 2 hours. After this time, the mixture was cooled to room temperature and concentrated to dryness under reduced pressure to afford the product, which was purified by prep. HPLC over a Phenomenex Gemini-NX C18, 75×30 mm×3 μm column (eluent: 20% to 50% (v/v) ACN and H₂O with 0.05% NH₃·H₂O and 10 mM NH₄HCO₃). The pure fractions were combined, and the solvents were removed under vacuum. The residue was re-suspended in water (20 mL) and the resulting mixture was lyophilized to dryness to remove the solvent residue completely to give title compound (35 mg, 33%) as a white solid. LCMS (ESI): mass calcd. for C₂₀H₁₈N₈O₂ 374.15 m/z, found 375.1 [M+H]⁺. ¹H NMR (400 MHz, DMSO-d₆) δ 11.99 (br s, 1H), 9.15 (s, 1H), 8.59-8.53 (m, 1H), 8.34-8.29 (m, 1H), 8.05-7.99 (m, 2H), 7.92-7.85 (m, 1H), 7.64-7.57 (m, 1H), 7.48-7.42 (m, 1H), 7.21 (dd, J=4.6, 7.9 Hz, 1H), 6.28 (br s, 1H), 3.53-3.46 (m, 1H), 3.44-3.40 (m, 1H), 2.87 (s, 3H), 2.46-2.38 (m, 1H), 2.35-2.26 (m, 1H).

Step B. (R)-3-(3-(4-(1H-pyrrolo[2,3-b]pyridin-3-yl)-1H-1,2,3-triazol-1-yl)phenyl)-3-hydroxy-1-methylpyrrolidin-2-one. The (R) and (S)-enantiomers of (R,S)-3-(3-(4-(1H-pyrrolo[2,3-b]pyridin-3-yl)-1H-1,2,3-triazol-1-yl)phenyl)-3-hydroxy-1-methylpyrrolidin-2-one (30 mg, 0.080 mmol) were separated by SFC over DAICEL CHIRALPAK AS 250 mm×30 mm×10 μm column (isocratic elution: EtOH (containing 0.1% of 25% aq. NH₃): supercritical CO₂, 45%: 55% to 45%: 55% (v/v)). The first eluting enantiomer was the title compound (10.2 mg, 34%, R_{T} of SFC=3.575 min) as a colorless solid. LC-MS (ESI): mass calcd. for C₂₀H₁₈N₆O₂ 374.15 m/z found 375.1 [M+H]⁺. ¹H NMR (400 MHz, DMSO-d₆) δ 11.99 (br s, 1H), 9.16 (5, 1H), 8.59-8.54 (m, 1H), 8.35-8.29 (m, 1H), 8.05-7.99 (m, 2H), 7.92-7.86 (m, 1H), 7.64-7.57 (m, 1H), 7.48-7.43 (m, 1H), 7.22 (dd, J=4.8, 7.9 Hz, 1H), 6.30 (br s, 1H), 3.54-3.47 (m, 2H), 2.87 (s, 3H), 2.46-2.39 (1m, 1H), 2.35-2.27 (m, 1H). The second eliding enantiomer (S)-3-(3-(4-(1H-pyrrolo[2,3-b]pyridin-3-yl)-1H-1,2,3-triazol-1-yl)phenyl)-3-hydroxy-1-methylpyrrolidin-2-one (9.6 mg, 32%, R_{T} of SFC=4.341 min) was obtained as a colorless solid. LC-MS (ESI): mass calcd. for C₂₀H₁₈N₆O₂ 374.15 m/z found 375.1 [M+H]⁺. ¹H NMR (400 MHz, DMSO-d6) δ 11.99 (br s, 1H), 9.15 (s, 1H), 8.59-8.54 (m, 1H), 8.34-8.29 (m, 1H), 8.05-8.00 (m, 2H), 7.92-7.85 (m, 1H), 7.64-7.57 (m, 1H), 7.48-7.43 (m, 1H), 7.22 (dd, J=4.6, 8.1 Hz, 1H), 6.29 (br s, 1H), 3.53-3.47 (m, 2H), 2.87 (s, 3H), 2.46-2.39 (m, 1H), 2.34-2.27 (m, 1H).

### Example 124. (R)-3-(3-(4-(1H-Pyrrolo[2,3-b]pyridin-3-yl)-1H-imidazol-1-yl)phenyl)-3-hydroxy-1-methylpyrrolidin-2-one

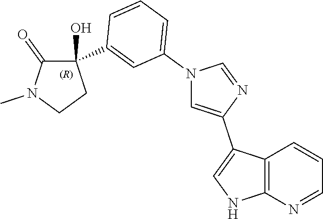

Step A. (R,S)-3-(3-(4-(1H-pyrrolo[2,3-b]pyridin-3-yl)-1H-imidazol-1-yl)phenyl)-3-hydroxy-1-methylpyrrolidin-2-one. Sodium hydroxide (6.3 mL, 13 mmol, 2.0 M in H₂O) was added to a mixture of 3-hydroxy-1-methyl-3-(3-(4-(1-tosyl-1H-pyrrolo[2,3-b]pyridin-3-yl)-1H-imidazol-1-yl)phenyl)pyrrolidin-2-one (Intermediate 181, 1.1 g, 2.1 mmol) and 1,4-dioxane (10 mL). The resultant mixture was heated at 60° C. for 3 hours. After this time, the mixture was cooled to room temperature, diluted with water (10 mL), and extracted with ethyl acetate (15 mL×3). The combined organic extracts was dried over anhydrous Na₂SO₄, filtered, and concentrated to dryness under reduced pressure to afford the compound, which was purified by prep-HPLC over Xtimate C18 150×40 mm×5 μm column (eluent: 15% to 45% (v/v) MeCN and water (0.05% NH₃H₂O)). The pure fractions were combined and the solvents were removed under vacuum. The residue was re-suspended in water (10 mL) and the resulting mixture was lyophilized to dryness to remove the solvent residue completely to afford product (330 mg, 16%) as a white solid. LCMS (ESI): mass calcd. for C₂₁H₁₉N₅O₂ 373.15 m/z, found 374.1 [M+1]⁺.

Step B. (R)-3-(3-(4-(1H-pyrrolo[2,3-b]pyridin-3-yl)-1H-imidazol-1-yl)phenyl)-3-hydroxy-1-methylpyrrolidin-2-one. The (R) and (S)-enantiomers of (R,S)-3-(3-(4-(1H-pyrrolo[2,3-b]pyridin-3-yl)-1H-imidazol-1-yl)phenyl)-3-hydroxy-1-methylpyrrolidin-2-one (330 mg, 0.884 mmol) were separated by SFC over DAICEL CHIRALPAK AD 250×30 mm×10 μm column (isocratic elution: EtOH (containing 0.1% of 25% aq. NH₃): supercritical CO₂, 45%: 55% to 45%: 55% (v/v)) to provide two enantiomers. The first eluting enantiomer provided the title compound (99.4 mg, 29%, Rr of SFC=1.272 min) as a white solid. LCMS (ESI): mass calcd. for C₂₁H₁₉N₅O₂ 373.15 m/z, found 374.1 [M+1]⁺. ¹H NMR (400 MHz, DMSO-d₆) δ 11.82-11.63 (m, 1H), 8.55-8.48 (m, 1H), 8.31-8.28 (m, 1H), 8.26 (dd, J=1.4, 4.6 Hz, 1H), 8.10-8.05 (m, 1H), 7.85-7.80 (m, 1H), 7.71-7.62 (m, 2H), 7.54-7.47 (m, 1H), 7.37-7.31 (m, 1H), 7.14 (dd, J=4.8, 7.8 Hz, 1H), 6.18 (s, 1H), 3.51-3.38 (m, 2H), 2.86 (s, 3H), 2.47-2.43 (m, 1H), 2.32-2.24 (m, 1H). The second eluting enantiomer, (S)-3-(3-(4-(1H-pyrrolo[2,3-b]pyridin-3-yl)-1H-imidazol-1-yl)phenyl)-3-hydroxy-1-methylpyrrolidin-2-one (99.7 mg, 30%, R_{T} of SFC=1.881 min) was obtained as a white solid. LCMS (ESI): mass calcd. for C₂₁H₁₉N₅O₂ 373.15 m/z, found 374.1 [M+1]⁺. ¹H NMR (400 MHz, DMSO-d₆) δ 11.72 (br s, 1H), 8.52 (d, J=8.0 Hz, 1H), 8.30 (s, 1H), 8.26 (d, J=4.5 Hz, 1H), 8.08 (s, 1H), 7.82 (s, 1H), 7.71-7.63 (m, 2H), 7.54-7.46 (m, 1H), 7.36-7.30 (m, 1H), 7.14 (dd, J=4.5, 7.8 Hz, 1H), 6.18 (s, 1H), 3.51-3.40 (m, 2H), 2.86 (s, 3H), 2.47-2.42 (m, 1H), 2.32-2.24 (m, 1H).

### Example 125. (R)-3-Hydroxy-1-methyl-3-(3-(1-methyl-5-(1H-pyrrolo[2,3-b]pyridin-3-yl)-1H-imidazol-2-yl)phenyl)pyrrolidin-2-one

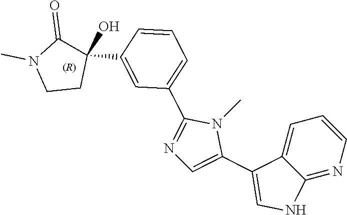

Sodium hydroxide (0.80 mL, 1.6 mmol, 2.0 M in H₂O) was added to a mixture of (R)-3-hydroxy-1-methyl-3-(3-(1-methyl-5-(1-tosyl-1H-pyrrolo[2,3-b]pyridin-3-yl)-1H-imidazol-2-yl)phenyl)pyrrolidin-2-one (Intermediate 182, 145 mg, 0.268 mmol) and 1,4-dioxane (2 mL). The mixture was heated at 60° C. for 3 hours. After this time, the mixture was cooled to room temperature, diluted with water (2 mL), and extracted with ethyl acetate (2 mL×3). The combined organic extracts was dried over Na₂SO₄, filtered, and concentrated to dryness under reduced pressure to afford the product, which was purified by prep-HPLC over Phenomenex Gemini-NX C18 75×30 mm×3 μm column (eluent: 18% to 48% (v/v) MeCN and water (0.05% NH₃H₂O)). The pure fractions were combined and the solvents were removed under vacuum. The residue was re-suspended in water (5 mL) and the resulting mixture was lyophilized to dryness to remove the solvent residue completely to afford the title compound (17.1 mg, 16%) as a colorless solid. LCMS (ESI): mass calcd. for C₂₂H₂₁N₅O₂ 387.43 m/z, found 388.1 [M+1]⁺. ¹H NMR (400 MHz, DMSO-d₆) δ 12.07 (br s, 1H), 8.34-8.29 (m, 1H), 8.14-8.08 (m, 1H), 7.80-7.76 (m, 1H), 7.75-7.71 (m, 1H), 7.67-7.60 (m, 1H), 7.52-7.46 (m, 1H), 7.46-7.41 (m, 1H), 7.22 (s, 1H), 7.20-7.15 (m, 1H), 6.15 (s, 1H), 3.69 (s, 3H), 3.50-3.45 (m, 2H), 2.86 (s, 3H), 2.42-2.34 (m, 1H), 2.33-2.24 (m, 1H).

### Example 126. (R)-6-(3-(2-(1H-Pyrrolo[2,3-b]pyridin-3-yl)thiazol-4-yl)phenyl)-3-methyl-5,6-dihydro-4H-cyclopenta[d]isoxazol-6-ol

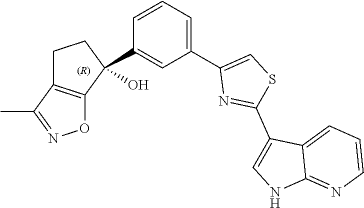

(R,S)-6-(3-(2-(1H-pyrrolo[2,3-b]pyridin-3-yl)thiazol-4-yl)phenyl)-3-methyl-5,6-dihydro-4H-cyclopenta[d]isoxazol-6-ol. Sodium hydroxide (7.46 mL, 14.9 mmol, 2.0 M in H₂O) was added to a mixture of 3-methyl-6-(3-(2-(1-(phenylsulfonyl)-1H-pyrrolo[2,3-b]pyridin-3-yl)thiazol-4-yl)phenyl)-5,6-dihydro-4H-cyclopenta[d]isoxazol-6-ol (Intermediate 186, 1.38 g, 2.49 mmol) in 1,4-dioxane (25 mL). The mixture was stirred for 2 hours at 60° C. After this time, the mixture was cooled to room temperature and concentrated under reduced pressure to afford a residue, which was initially purified by FCC (eluent: EtOAc:petroleum ether=0:1 to 3:1) to afford the product, which was further purified by prep-HPLC over a Phenomenex Gemini NX-C18 75×30 mm×3 μm column (eluent: 30% to 60% (v/v) ACN and water (0.04% NH₃H₂O+10 mM NH₄HCO₃)) to provide (R,S)-6-(3-(2-(1H-pyrrolo[2,3-b]pyridin-3-yl)thiazol-4-yl)phenyl)-3-methyl-5,6-dihydro-4H-cyclopenta[d]isoxazol-6-ol as a white solid. The (R) and (S)-enantiomers of (R,S)-6-(3-(2 (1H-pyrrolo[2,3-b]pyridin-3-yl)thiazol-4-yl)phenyl)-3-methyl-5,6-dihydro-4H-cyclopenta[d]isoxazol-6-ol products were separated by SFC over a DAICEL CHIRALCEL OD-H 250 mm×30 mm×5 μm column. An isocratic elution was used throughout the purification which consisted of EtOH (containing 0.1% of 25% aq. NH₃): supercritical CO₂=40%: 60%. The first eluting enantiomer, the title compound (R)-6-(3-(2-(1H-pyrrolo[2,3-b]pyridin-3-yl)thiazol-4-yl)phenyl)-3-methyl-5,6-dihydro-4H-cyclopenta[d]isoxazol-6-ol. (61.5 mg, 71%) was obtained as colorless solid. LCMS (ESI): mass calcd. for C₂₃H₁₈N₄O₂S 414.11 m/z, found 415.1 [M+1]⁺. ¹H NMR (400 MHz, DMSO-d₆) δ 8.63 (d, J=7.7 Hz, 1H), 8.41-8.34 (m, 1H), 8.29 (s, 1H), 8.10 (s, 1H), 8.03-7.93 (m, 2H), 7.52-7.45 (m, 1H), 7.39-7.34 (m, 1H), 7.33-7.26 (m, 1H), 6.50 (br s, 1H), 2.99-2.90 (m, 2H), 2.75-2.66 (m, 1H), 2.61-2.55 (m, 1H), 2.28 (s, 3H). The second eluting enantiomer, (S)-6-(3-(2-(1H-pyrrolo[2,3-b]pyridin-3-yl)thiazol-4-yl)phenyl)-3-methyl-5,6-dihydro-4H-cyclopenta[d]isoxazol-6-ol (76.1 mg, 7.4%) was obtained as colorless solid. LCMS (ESI): mass calcd. for C₂₃H₁₈N₄O₂S 414.11 m/z, found 415.1 [M+1]⁺. ¹H NMR (400 MHz, DMSO-d₆) δ 13.31-11.10 (m, 1H), 8.63 (d, J=7.9 Hz, 1H), 8.40-8.34 (m, 1H), 8.28 (s, 1H), 8.09 (s, 1H), 8.00-7.93 (m, 2H), 7.52-7.44 (m, 1H), 7.36 (d, J=7.7 Hz, 1H), 7.31 (dd, J=4.6, 7.9 Hz, 1H), 6.69-6.26 (m, 1H), 3.00-2.88 (m, 2H), 2.75-2.67 (m, 1H), 2.62-2.54 (m, 1H), 2.27 (s, 3H).

### Example 127. (R)-3-(3-(4-(1H-Pyrrolo[2,3-b]pyridin-4-yl)thiazol-2-yl)phenyl)-3-hydroxy-1-methylpyrrolidin-2-one

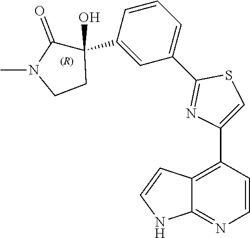

To a microwave tube containing 4-(4,4,5,5-tetramethyl-1,3,2-dioxaborolan-2-yl)-1H-pyrrolo[2,3-b]pyridine (43 mg, 0.177 mmol), ((R)-3-(3-(4-bromothiazol-2-yl)phenyl)-3-hydroxy-1-methylpyrrolidin-2-one (Intermediate 188, 50 mg, 0.142 mmol), K₂CO₃ (2M aqueous, 0.283 mL, 0.566 mmol), and 1,4-dioxane (2.8 mL) was added tetrakis(triphenylphosphine)palladium(0) (16 mg, 0.0142 mmol). The resultant mixture was heated in aluminum heating mantle at 80° C. for 3 h. After this time, the mixture was cooled to room temperature, and concentrated to dryness. The resulting residue was purified by FCC (100% DCM increasing to 7% MeOH in EtOAc over 20 column volumes) afford (R)-3-(3-(4-(1H-pyrrolo[2,3-b]pyridin-4-yl)thiazol-2-yl)phenyl)-3-hydroxy-1-methylpyrrolidin-2-one (510 mg) as a white solid (29 mg, 52%). LC-MS (ESI): mass calcd. for C₂₁H₁₈N₄O₂S 390.12 m/z found 391.1 [M+H]⁺. ¹H NMR (400 MHz, MeOD-d₄) δ 8.29 (d, J=5.2 Hz, 1H), 8.30-8.23 (m, 3H), 8.23 (s, 1H), 8.08-8.04 (m, 1H), 7.77 (d, J=5.2 Hz, 1H), 7.58-7.55 (m, 2H), 7.53 (d, J=3.5 Hz, 1H), 7.19 (d, J=3.5 Hz, 1H), 3.64-3.58 (m, 1H), 3.56-3.50 (m, 1H), 3.04 (s, 3H), 2.58-2.51 (m, 1H), 2.47 (ddd, J=13.4, 8.2, 6.3 Hz, 1H).

### Example 128. (R)-3-Hydroxy-1-methyl-3-(3-(4-(1-methyl-1H-indazol-6-yl)thiazol-2-yl)phenyl)pyrrolidin-2-one

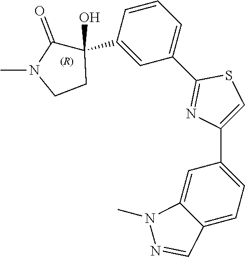

The title compound (39 mg, 71%) was prepared in a manner analogous to that as described in Example 127 using (1-methyl-1H-indazol-6-yl)boronic acid in place of 4 (4,4,5,5-tetramethyl-1,3,2-dioxaborolan-2-yl)-1H-pyrrolo[2,3-b]pyridine. The resulting residue was purified by preparative HPLC (XBridge OBD C18 5 μm, 50×100 mm column using a 0 to 95% gradient of ACN/20 mM NH₄OH in H₂O over 16 min. Detection, UV at λ=220-254 nM). LC-MS (ESI): mass calcd. for C₂₂H₂₀N₄O₂S 404.13 m/z found 405.1 [M+H]⁺. ¹H NMR (500 MHz, MeOD-d₄) δ 8.24 (q, J=1.1 Hz, 1H), 8.24-8.19 (m, 1H), 8.04-8.00 (m, 2H), 7.95 (s, 1H), 7.81 (t, J=1.3 Hz, 2H), 7.53-7.50 (m, 2H), 4.14 (s, 3H), 3.59 (ddd, J=10.2, 8.2, 4.6 Hz, 1H), 3.50 (ddd, J=10.2, 7.6, 6.0 Hz, 1H), 3.01 (s, 3H), 2.53 (ddd, J=13.5, 7.6, 4.6 Hz, 1H), 2.44 (ddd, J=13.4, 8.2, 6.0 Hz, 1H).

### Example 129. (R)-3-(3-(4-(6-Amino-1H-pyrrolo[2,3-b]pyridin-4-yl)thiazol-2-yl)phenyl)-3-hydroxy-1-methylpyrrolidin-2-one

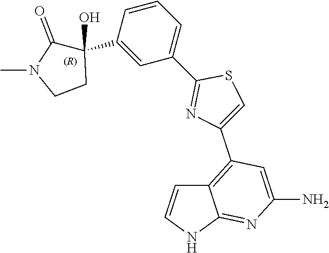

The title compound (32 mg, 55%) was prepared in a manner analogous to that as described in Example 127 using 4-(4,4,5,5-tetramethyl-1,3,2-dioxaborolan-2-yl)-1H-pyrrolo[2,3-b]pyridin-6-amine in place of 4-(4,4,5,5-tetramethyl-1,3,2-dioxaborolan-2-yl)-1H-pyrrolo[2,3-b]pyridine. The resulting residue was purified by preparative HPLC (XBridge OBD C18 5 μm, 50×100 mm column using a 0 to 95% gradient of ACN/20 mM NH₄OH in H₂O over 16 min. Detection, UV at λ=220-254 nM). LC-MS (ESI): mass calcd. for C₂₁H₁₉N₅O₂S 405.13 m/z found 406.2 [M+H]⁺. ¹H NMR (400 MHz, MeOD-d₄) δ 8.24-8.16 (m, 1H), 8.05 (s, 1H), 8.03-7.94 (m, 1H), 7.57-7.47 (m, 2H), 7.08 (m, 2H), 6.86 (d, J=3.5 Hz, 1H), 3.61-3.53 (m, 1H), 3.49 (ddd, J=10.2, 7.5, 6.2 Hz, 1H), 3.01 (s, 3H), 2.56-2.37 (m, 2H).

### Example 130. (R)-3-(3-(4-(5-Chloro-1H-pyrrolo[2,3-b]pyridin-4-yl)thiazol-2-yl)phenyl)-3-hydroxy-1-methylpyrrolidin-2-one

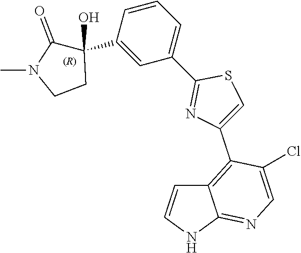

The title compound (37 mg, 62%) was prepared in a manner analogous to that as described in Example 127 using 1-(4-(4,4,5,5-tetramethyl-1,3,2-dioxaborolan-2-yl)pyridin-2-yl)ethan-1-one in place of 4-(4,4,5,5-tetramethyl-1,3,2-dioxaborolan-2-yl)-1H-pyrrolo[2,3-b]pyridine. The resulting residue was purified by preparative HPLC (XBridge OBD C18 5 μm, 50×100 mm column using a 0 to 95% gradient of ACN/20 mM NH₄OH in H₂O over 16 min. Detection, UV at λ=220-254 nM). LC-MS (ESI): mass calcd. for C₂₁H₁₇ClN₄O₂S 424.08 m/z found 425.1 [M+H]⁺. ¹H NMR (400 MHz, MeOD-d₄) δ 8.31 (s, 1H), 8.16 (t, J=1.7 Hz, 1H), 8.11 (s, 1H), 7.99 (dt, J=7.2, 1.7 Hz, 1H), 7.60-7.45 (m, 3H), 6.77 (d, J=3.5 Hz, 1H), 3.57 (ddd, J=10.2, 8.2, 4.5 Hz, 1H), 3.53-3.44 (m, 1H), 2.99 (s, 3H), 2.56-2.40 (m, 2H).

### Example 131 (R)-3-(3-(4-(1H-Indazol-4-yl)thiazol-2-yl)phenyl)-3-hydroxy-1-methylpyrrolidin-2-one

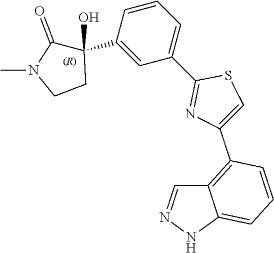

The title compound (28 mg, 53%) was prepared in a manner analogous to that as described in Example 127 using (1H-indazol-4-yl)boronic acid in place of 4-(4,4,5,5-tetramethyl-1,3,2-dioxaborolan-2-yl)-1H-pyrrolo[2,3-b]pyridine. The resulting residue was purified by preparative HPLC (XBridge OBD C18 5 μm, 50×100 mm column using a 0 to 95% gradient of ACN/20 mM NH₄OH in H₂O over 16 min. Detection, UV at λ=220-254 nM). LC-MS (ESI): mass calcd. for C₂₁H₁₈N₄O₂S 390.12 m/z found 391.1 [M+H]⁺. ¹H NMR (400 MHz, MeOD-d₄) δ 8.62 (d, J=1.0 Hz, 1H), 8.12 (dd, J=2.0, 0.9 Hz, 1H), 7.97-7.88 (m, 2H), 7.65 (dd, J=7.1, 0.9 Hz, 1H), 7.50-7.34 (m, 4H), 3.49 (ddd, J=10.2, 8.2, 4.5 Hz, 1H), 3.40 (ddd, J=10.2, 7.5, 6.2 Hz, 1H), 2.48-2.28 (m, 2H).

### Example 132. (R)-3-(3-(4-(5-Fluoro-1H-pyrrolo[2,3-b]pyridin-4-yl)thiazol-2-yl)phenyl)-3-hydroxy-1-methylpyrrolidin-2-one

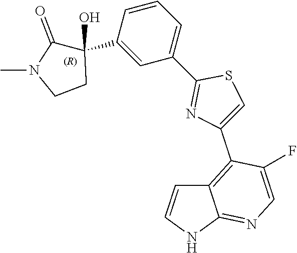

The title compound (46 mg, 80%) was prepared in a manner analogous to that as described in Example 127 using 5-fluoro-4-(4,4,5,5-tetramethyl-1,3,2-dioxaborolan-2-yl)-1-(triisopropylsilyl)-1H-pyrrolo[2,3-b]pyridine in place of 4-(4,4,5,5-tetramethyl-1,3,2-dioxaborolan-2-yl)-1H-pyrrolo[2,3-b]pyridine. The resulting residue was purified by preparative HPLC (XBridge OBD C18 5 μm, 50×100 mm column using a 0 to 95% gradient of ACN/20 mM NH₄OH in H₂O over 16 min. Detection, UV at λ=220-254 nM). LC-MS (ESI): mass calcd. for C₂₁H₁₇FN₄O₂S 408.11 m/z found 409.1 [M+H]⁺. ¹H NMR (400 MHz, MeOD-d₄) δ 8.27-8.15 (m, 3H), 8.09-7.98 (m, 1H), 7.59-7.50 (m, 3H), 7.37 (d, J=3.5 Hz, 1H), 3.59 (ddd, J=10.2, 8.2, 4.5 Hz, 1H), 3.50 (ddd, J=10.2, 7.5, 6.4 Hz, 1H), 3.02 (s, 3H), 2.56-2.41 (m, 2H).

Compounds of the invention were tested in biological assays. The results of the assays are presented in Table 6 below which is entitled Results of Biological Assays. The results are presented as an average of values obtained.

### Assay 1

### Inhibition of Auto-Phosphorylation of Recombinant Human NF-kappaB-Inducing Kinase (NIK/MAP3K14) Activity (AlphaScreen®)

NIK/MAP3K14 auto-phosphorylation activity was measured using the AlphaScreen® (αscreen) format (Perkin Elmer). All compounds tested were dissolved in dimethyl sulfoxide (DMSO) and further dilutions were made in assay buffer. The final DMSO concentration was 0.7% (v/v) in assays. The assay buffer was 50 mM Tris pH 7.5 containing 1 mM EGTA (ethylene glycol tetraacetic acid), 1 mM DTT (dithiothreitol), 0.1 mM Na₃VO₄, 5 mM MgCl₂, and 0.01% Tween® 20. The assays were carried out in 384 well Proxiplates (Perkin Elmer). The incubations consisted of the compound, 5 μM Adenosine-5′-triphosphate (ATP), and 1 nM NIK/MAP3K14. Incubations were initiated by the addition of GST-tagged NIK/MAP3K14 enzyme, carried out for 2 h at 25° C. and terminated by addition of stop buffer containing anti-phospho-IKK Ser176/180 antibody. Protein A Acceptor and Glutathione-Donor beads were added before reading using an EnVision Multilabel Plate Reader (Perkin Elmer). The signal obtained in the wells was normalized using high (full enzyme activity, 0.7% DMSO) and low controls (no enzyme activity, 0.7% DMSO, no ATP). IC₅₀'s were determined by fitting a sigmoidal curve to % inhibition of control versus Log₁₀ compound concentration.

### Assay 2

### Effect of Compounds on p-IKKα Levels in L363 (NIK Translocated Multiple Myeloma) Cells

All compounds tested were dissolved and serially diluted in DMSO, 1:3 dilution for 11 points in an Echo compatible plate. 100% DMSO was added to columns 12 and 24 of the plate to serve as high and low signal controls. This compound plate was used to spot 20 nL of compound or DMSO into a Greiner 384 well TC plate (781080). The final DMSO concentration was 0.3% (v/v) in cell assays. Human L363 cells (ATCC) were cultured in RPMI1640 medium supplemented with GlutaMax, non-essential amino acids, sodium pyruvate and 10% fetal bovine serum. Cells were routinely maintained at densities of 0.2×10⁸ cells per ml-2×10⁶ cells per mL at 37° C. in a humidified 5% CO₂ atmosphere incubator. Cells were passaged twice a week splitting back to obtain the low density. The day before the assay, cells were washed twice in HBSS (Hank's Balanced Salt Solution), resuspended in Dulbecco's Modified Eagle Medium (DMEM)+0.5% IgG and protease free BSA (Jackson Immuno Research Laboratories), +/−250 ng/ml recombinant human B-cell activating factor (BAFF/BLyS/TNFSF13B) and incubated overnight at 37° C. in a humidified 5% CO₂ atmosphere (bulk stimulation with or without BAFF). The next day, the cell concentration was adjusted to 1×10⁷ cells/ml in DMEM+/−250 ng/ml BAFF+/10 μM MG132 and plated at 10 μU well into compound or DMSO spotted 384 well TC plates. Seeded cells were incubated at 37° C. in a humidified 5% CO₂ atmosphere for 6 h. After 6 h, the plates were removed from the incubator and cell lysis was achieved by the addition of 2.5 μl 5×lysis buffer containing protease and phosphatase inhibitors, followed by shaking on a plate shaker at room temperature for 15 min. At the end of this incubation, lysed cells were sequentially treated and incubated with acceptor and donor bead mixes according to the manufacturer's protocol for a 1 plate/2-incubation suspension cell assay (AlphaLISA SureFire Ultra p-IKKa (Ser 176/180) Assay Kit (Perkin Elmer). Plates were read using an EnVision Multilabel Plate Reader (Perkin Elmer). Within an experiment, a concentration response curve for each compound was run in duplicate. The signal obtained in the test wells was normalized using high signal (BAFF stimulated cells, DMSO, MG132) and low signal (unstimulated cells, DMSO) controls. To determine the IC₅₀, a sigmoidal curve was fitted to the plot of % inhibition versus Log₁₀ compound concentration.

Table 6 below provides IC₅₀ data for certain compounds described herein on NIK inhibition.

TABLE 6
Results of Biological Assays

| | Assay 1 IC₅₀ | Assay 2 IC₅₀ |
|---|---|---|
| Example | (nM) | (nM) |
| 1 | C | C |
| 2 | A | B |
| 3 | B | C |
| 4 | C | C |
| 5 | A | B |
| 6 | B | C |
| 7 | A | B |
| 8 | A | C |
| 9 | B | C |
| 10 | A | A |
| 11 | A | A |
| 12 | B | C |
| 13 | A | C |
| 14 | C | C |
| 15 | A | B |
| 16 | A | B |
| 17 | A | A |
| 18 | C | C |
| 19 | C | C |
| 20 | A | A |
| 21 | B | C |
| 22 | A | B |
| 23 | B | C |
| 24 | A | A |
| 25 | A | B |
| 26 | A | A |
| 27 | A | A |
| 28 | A | B |
| 29 | A | A |
| 30 | A | C |
| 31 | B | C |
| 32 | A | A |
| 33 | A | C |
| 34 | C | C |
| 35 | C | C |
| 36 | A | C |
| 37 | C | C |
| 38 | A | A |
| 39 | C | C |
| 40 | A | B |
| 41 | A | A |
| 42 | B | C |
| 43 | C | C |
| 44 | C | C |
| 45 | C | C |
| 46 | C | C |
| 47 | A | B |
| 48 | C | C |
| 49 | A | B |
| 50 | A | C |
| 51 | A | C |
| 52 | B | C |
| 53 | A | C |
| 54 | A | B |
| 55 | C | C |
| 56 | B | C |
| 57 | B | C |
| 58 | B | C |
| 59 | A | A |
| 60 | C | C |
| 61 | C | C |
| 62 | A | A |
| 63 | A | A |
| 64 | C | C |
| 65 | A | C |
| 66 | A | C |
| 67 | A | C |
| 68 | A | C |
| 69 | B | C |
| 70 | C | C |
| 71 | B | C |
| 72 | A | C |
| 73 | A | C |
| 74 | C | C |
| 75 | B | C |
| 76 | B | C |
| 77 | C | C |
| 78 | C | C |
| 79 | C | C |
| 80 | C | C |
| 81 | A | A |
| 82 | B | C |
| 83 | A | B |
| 84 | B | C |
| 85 | C | C |
| 86 | C | C |
| 87 | A | C |
| 88 | A | C |
| 89 | C | C |
| 90 | C | C |
| 91 | C | C |
| 92 | A | C |
| 93 | A | C |
| 94 | A | C |
| 95 | A | C |
| 96 | A | C |
| 97 | A | B |
| 98 | C | C |
| 99 | A | C |
| 100 | C | C |
| 101 | B | C |
| 102 | C | C |
| 103 | C | C |
| 104 | C | C |
| 105 | C | C |
| 106 | C | C |
| 107 | C | C |
| 108 | B | C |
| 109 | A | C |
| 110 | B | C |
| 111 | B | C |
| 112 | C | C |
| 113 | B | C |
| 114 | A | B |
| 115 | C | C |
| 116 | A | C |
| 117 | B | C |
| 118 | B | C |
| 119 | C | C |
| 120 | C | C |
| 121 | A | C |
| 122 | A | C |
| 123 | C | C |
| 124 | C | C |
| 125 | B | C |
| 126 | A | C |
| 127 | A | C |
| 128 | C | C |
| 129 | C | C |
| 130 | C | C |
| 131 | C | C |
| 132 | C | C |

Assay IC₅₀ ranges: A ≤ 100 nM, 100 nM < B ≤ 500 nM, C > 500 nM.

1. A compound of Formula I or a pharmaceutically acceptable salt thereof, 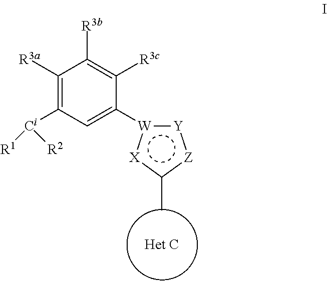
wherein
C¹ is —C(OH)—,
R¹ is C₁-C₃ alkyl;
R² is a 5 or 6 membered substituted or unsubstituted heteroaryl;
or R¹ and R² can be taken together with the carbon atom to which they are attached to form a substituted or unsubstituted heterocycle;
R^{3a}, R^{3b} and R^{3c} are independently H or C₁-C₃ alkyl; 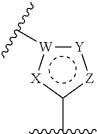
is a substituted or unsubstituted heteroaryl,
X, Y and Z are independently selected from O, S, N, N—R⁴ or C—R⁵;
W is N or C;
R⁴ is H or —C₁-C₃ alkyl; and
R⁵ is H, halo, CN, —C₁-C₃ alkyl or CH₂OH;
Het C is selected from the group consisting of 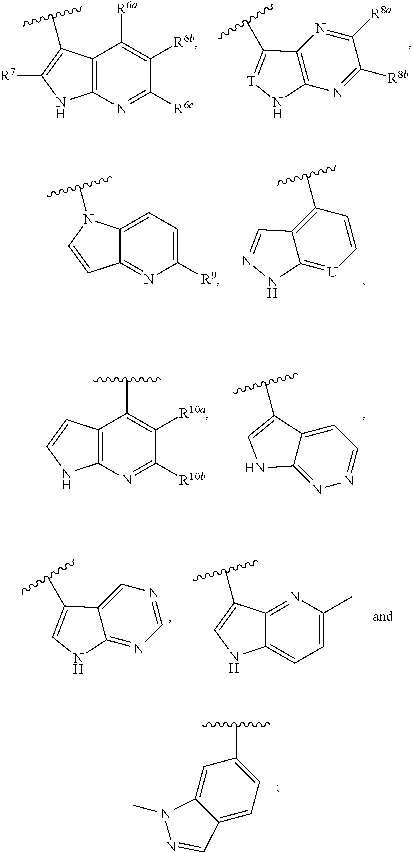
R^{6a}, R^{6b}, R^{6c} is independently H, halo, —C₁-C₃ alkyl, —C₁-C₃ haloalkyl, —CN, —CO₂H, —OC₁-C₃ alkyl, —SO₂CH₃ or —NH₂;
R⁷ is H, D, halo or —C₁-C₃ alkyl;
R^{8a}, R^{8b}, and R^{8c} is independently H, D, halo or —C₁-C₃ alkyl;
R⁹, R^{10a} and R^{10b} is independently H or NH₂;
T is N or C—R^{8c}; and
U is N or CH.

2. The compound of claim 1, wherein R¹ is —C₁-C₃ alkyl,

3. (canceled)

4. The compound of claim 1, wherein R² is a 5-8 membered heteroaryl containing one or more nitrogen atoms.

5. The compound of claim 4, wherein R² is pyridinyl, thiazolyl, or imidazolyl, and wherein the heteroaryl ring is unsubstituted or substituted with —C₁-C₃ alkyl.

6. (canceled)

7. The compound of claim 1, wherein R¹ and R² are taken together with the carbon atom to which they are attached to form a substituted or unsubstituted heterocycle containing at least one nitrogen atom.

8. (canceled)

9. (canceled)

10. The compound of claim 1, wherein R^{3a} is CH₃ and R^{3b} and R^{3c} are H, R^{3b} is CH₃ and R^{3a} and R^{3c} are H, or R^{3c} is CH₃ and R^{3a} and R^{3b} are H.

11-12. (canceled)

13. The compound of claim 1, wherein W is C.

14. The compound of claim 1, wherein X is N.

15. The compound of claim 1, wherein Y is C—R⁵.

16. The compound of claim 1, wherein 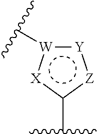
is a substituted or unsubstituted ring selected from imidazolyl, pyrazolyl, triazolyl, furanyl, thiazolyl, and oxazolyl.

17-19. (canceled)

20. The compound of ene of claim 1, wherein Het C is

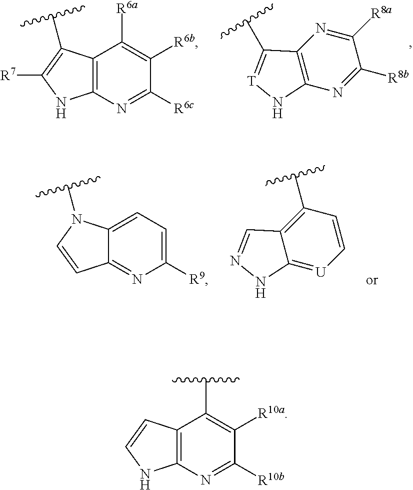

21-23. (canceled)

24. The compound of claim 1, wherein the absolute stereochemistry of the carbon atom of C¹ is (R) or (S).

25. (canceled)

26. A compound selected from
(S)-7-(3-(2-(1H-Pyrrolo[2,3-b]pyridin-3-yl)thiazol-4-yl)phenyl)-6,7-dihydro-5H-cyclopenta[b]pyridin-7-ol;
(R)-7-(3-(2-(1H-Pyrrolo[2,3-b]pyridin-3-yl)thiazol-4-yl)phenyl)-6,7-dihydro-5H-cyclopenta[b]pyridin-7-ol;
(R)-3-(3-(2-(1H-Pyrrolo[2,3-b]pyridin-3-yl)thiazol-5-yl)phenyl)-3-hydroxy-1-methylpyrrolidin-2-one;
(R)-7-(3-(2-(7H-Pyrrolo[2,3-d]pyrimidin-5-yl)thiazol-4-yl)phenyl)-6,7-dihydro-5H-cyclopenta[b]pyridin-7-ol;
(S)-7-(3-(2-(7H-Pyrrolo[2,3-d]pyrimidin-5-yl)thiazol-4-yl)phenyl)-6,7-dihydro-5H-cyclopenta[b]pyridin-7-ol;
(R)-3-Hydroxy-1-methyl-3-(3-(2-(5-methyl-1H-pyrrolo[2,3-b]pyridin-3-yl)thiazol-4-yl)phenyl)pyrrolidin-2-one;
(R)-3-(3-(4-(1H-Pyrrolo[2,3-b]pyridin-3-yl)thiazol-2-yl)phenyl)-3-hydroxy-1-methylpyrrolidin-2-one;
(R)-7-(3-(2-(1H-Pyrrolo[2,3-b]pyridin-3-yl)thiazol-4-yl)phenyl)-6,7-dihydro-5H-pyrrolo[1,2-a]imidazol-7-ol;
(R)-7-(3-(4-(5H-Pyrrolo[2,3-b]pyrazin-7-yl)thiazol-2-yl)phenyl)-6,7-dihydro-5H-pyrrolo[1,2-a]imidazol-7-ol;
(S)-7-(3-(4-(5H-Pyrrolo[2,3-b]pyrazin-7-yl)thiazol-2-yl)phenyl)-6,7-dihydro-5H-pyrrolo[1,2-a]imidazol-7-ol;
(R)-7-(3-(2-(5H-Pyrrolo[2,3-b]pyrazin-7-yl)thiazol-4-yl)phenyl)-7H-pyrrolo[1,2-a]imidazol-7-ol;
(S)-7-(3-(2-(5H-Pyrrolo[2,3-b]pyrazin-7-yl)thiazol-4-yl)phenyl)-7H-pyrrolo[1,2-a]imidazol-7-ol;
(R)-7-(3-(4-(1H-Pyrrolo[2,3-b]pyridin-3-yl)thiazol-2-yl)phenyl)-6,7-dihydro-5H-pyrrolo[1,2-a]imidazol-7-ol;
(R)-3-(3-(2-(1H-Pyrrolo[2,3-b]pyridin-3-yl)thiazol-4-yl)phenyl)-2,3-dihydrofuro[3,2-b]pyridin-3-ol;
(R)-3-(3-(2-(5H-Pyrrolo[2,3-b]pyrazin-7-yl)thiazol-4-yl)phenyl)-2,3-dihydrofuro[3,2-b]pyridin-3-ol;
(S)-7-(3-(2-(5H-Pyrrolo[2,3-b]pyrazin-7-yl-2-ci)thiazol-4-yl)phenyl)-6,7-dihydro-5H-pyrrolo[1,2-a]imidazol-7-ol;
(R)-7-(3-(2-(7H-Pyrrolo[2,3-d]pyrimidin-5-yl)thiazol-4-yl)phenyl)-6,7-dihydro-5H-pyrrolo[1,2-a]imidazol-7-ol;
(S)-7-(3-(2-(7H-Pyrrolo[2,3-d]pyrimidin-5-yl)thiazol-4-yl)phenyl)-6,7-dihydro-5H-pyrrolo[1,2-a]imidazol-7-ol;
(R)-7-(3-(4-(1H-Pyrrolo[2,3-b]pyridin-3-yl)thiazol-2-yl)phenyl)-6,7-dihydro-5H-cyclopenta[b]pyridin-7-ol;
(S)-7-(3-(4-(1H-Pyrrolo[2,3-b]pyridin-3-yl)thiazol-2-yl)phenyl)-6,7-dihydro-5H-cyclopenta[b]pyridin-7-ol;
(R)-7-(3-(2-(5-Fluoro-1H-pyrrolo[2,3-b]pyridin-3-yl)thiazol-4-yl)phenyl)-6,7-dihydro-5H-pyrrolo[1,2-a]imidazol-7-ol;
(R)-7-(3-(2-(5-(Trifluoromethyl)-1H-pyrrolo[2,3-b]pyridin-3-yl)thiazol-4-yl)phenyl)-6,7-dihydro-5H-pyrrolo[1,2-a]imidazol-7-ol;
(R)-3-(4-(3-(7-Hydroxy-6,7-dihydro-5H-pyrrolo[1,2-a]imidazol-7-yl)phenyl)thiazol-2-yl)-1H-pyrrolo[2,3-b]pyridine-5-carbonitrile;
(R)-7-(3-(2-(5-Chloro-1H-pyrrolo[2,3-b]pyridin-3-yl)thiazol-4-yl)phenyl)-6,7-dihydro-5H-pyrrolo[1,2-a]imidazol-7-ol;
(R)-7-(3-(2-(5-(Methylsulfonyl)-1H-pyrrolo[2,3-b]pyridin-3-yl)thiazol-4-yl)phenyl)-6,7-dihydro-5H-pyrrolo[1,2-a]imidazol-7-ol;
(R)-7-(3-(2-(4-Fluoro-1H-pyrrolo[2,3-b]pyridin-3-yl)thiazol-4-yl)phenyl)-6,7-dihydro-5H-pyrrolo[1,2-a]imidazol-7-ol;
(R)-7-(3-(5-(1H-Pyrrolo[2,3-b]pyridin-3-yl)furan-3-yl)phenyl)-6,7-dihydro-5H-pyrrolo[1,2-a]imidazol-7-ol;
(S)-4-(3-(2-(1H-Pyrrolo[2,3-b]pyridin-3-yl)thiazol-4-yl)phenyl)-5,6-dihydro-4H-cyclopenta[d]thiazol-4-ol;
(R)-4-(3-(2-(1H-Pyrrolo[2,3-b]pyridin-3-yl)thiazol-4-yl)phenyl)-5,6-dihydro-4H-cyclopenta[d]thiazol-4-ol;
(R)-7-(3-(2-(1H-Pyrrolo[2,3-b]pyridin-3-yl)thiazol-4-yl)phenyl)-7H-cyclopenta[b]pyridin-7-ol;
(R)-7-(3-(2-(1H-Pyrrolo[2,3-b]pyridin-3-yl)thiazol-4-yl)phenyl)-6,6-dimethyl-6,7-dihydro-5H-cyclopenta[b]pyridin-7-ol.
(S)-8-(3-(2-(1H-Pyrrolo[2,3-b]pyridin-3-yl)thiazol-4-yl)phenyl)-5,6,7,8-tetrahydroimidazo[1,2-a]pyridin-8-ol;
(R)-8-(3-(2-(1H-Pyrrolo[2,3-b]pyridin-3-yl)thiazol-4-yl)phenyl)-5,6,7,8-tetrahydroimidazo[1,2-a]pyridin-8-ol;
(R)-3-(3-(2-(5H-Pyrrolo[2,3-b]pyrazin-7-yl)thiazol-4-yl)phenyl)-3-hydroxy-1-methylpyrrolidin-2-one;
(R)-7-(3-(2-(5H-Pyrrolo[2,3-b]pyrazin-7-yl)thiazol-4-yl)phenyl)-6,7-dihydro-5H-pyrrolo[1,2-a]imidazol-7-ol;
(S)-7-(3-(2-(5H-Pyrrolo[2,3-b]pyrazin-7-yl)thiazol-4-yl)phenyl)-6,7-dihydro-5H-pyrrolo[1,2-a]imidazol-7-ol;
(R)-3-(3-(2-(5H-Pyrrolo[2,3-b]pyrazin-7-yl)thiazol-4-yl)phenyl)-3-hydroxy-1-(2,2,2-trifluoroethyl)pyrrolidin-2-one;
(R)-3-(3-(2-(5H-Pyrrolo[2,3-b]pyrazin-7-yl)thiazol-4-yl)phenyl)-3-hydroxy-1-methylazetidin-2-one;
(R)-3-(3-(2-(1H-Pyrrolo[2,3-b]pyridin-3-yl)thiazol-4-yl)phenyl)-3-hydroxy-1-methylpyrrolidin-2-one;
(S)-3-(3-(2-(1H-Pyrrolo[2,3-b]pyridin-3-yl)thiazol-4-yl)phenyl)-3-hydroxy-1-methylpyrrolidin-2-one;
(R)-3-Hydroxy-3-(3-(2-(5-methoxy-1H-pyrrolo[2,3-b]pyridin-3-yl)thiazol-4-yl)phenyl)-1-methylpyrrolidin-2-one;
(R)-3-Hydroxy-3-(3-(5-(hydroxymethyl)-2-(1H-pyrrolo[2,3-b]pyridin-3-yl)thiazol-4-yl)phenyl)-1-methylpyrrolidin-2-one;
(R)-3-(4-(3-(3-Hydroxy-1-methyl-2-oxopyrrolidin-3-yl)phenyl)thiazol-2-yl)-1H-pyrrolo[2,3-b]pyridine-5-carboxylic acid;
(S)-3-(3-(2-(7H-Pyrrolo[2,3-d]pyrimidin-5-yl)thiazol-4-yl)phenyl)-3-hydroxy-1-methylpyrrolidin-2-one;
(R)-3-(3-(2-(7H-Pyrrolo[2,3-d]pyrimidin-5-yl)thiazol-4-yl)phenyl)-3-hydroxy-1-methylpyrrolidin-2-one;
(R)-3-(3-(2-(5H-Pyrrolo[2,3-b]pyrazin-7-yl)thiazol-4-yl)phenyl)-3-hydroxy-1-methylpiperidin-2-one;
(R)-3-(3-(4-(2-Fluoro-1H-pyrrolo[2,3-b]pyridin-3-yl)thiazol-2-yl)phenyl)-3-hydroxy-1-methylpyrrolidin-2-one;
(R)-3-(3-(2-(2-Fluoro-1H-pyrrolo[2,3-b]pyridin-3-yl)thiazol-4-yl)phenyl)-3-hydroxy-1-methylpyrrolidin-2-one;
(S)-4-(3-(4-(1H-Pyrrolo[2,3-b]pyridin-3-yl)thiazol-2-yl)phenyl)-5,6-dihydro-4H-cyclopenta[d]thiazol-4-ol;
(R)-4-(3-(4-(1H-Pyrrolo[2,3-b]pyridin-3-yl)thiazol-2-yl)phenyl)-5,6-dihydro-4H-cyclopenta[d]thiazol-4-ol;
(R)-1-(3-(2-(1H-Pyrrolo[2,3-b]pyridin-3-yl)thiazol-4-yl)phenyl)-1-(pyridin-2-yl)ethan-1-ol;
(R)-7-(3-(2-(5H-Pyrrolo[2,3-b]pyrazin-7-yl-6-d)thiazol-4-yl)phenyl)-6,7-dihydro-5H-pyrrolo[1,2-a]imidazol-7-ol;
(R)-7-(3-(2-(5H-Pyrrolo[2,3-b]pyrazin-7-yl-2-d)thiazol-4-yl)phenyl)-6,7-dihydro-5H-pyrrolo[1,2-a]imidazol-7-ol; and pharmaceutically acceptable salts thereof.

27. A compound as claimed in claim 0, wherein said compound is selected from
(R)-4-(3-(2-(1H-Pyrrolo[2,3-b]pyridin-3-yl)thiazol-4-yl)phenyl)-5,6-dihydro-4H-cyclopenta[d]thiazol-4-ol;
(R)-3-(3-(2-(1H-pyrrolo[2,3-b]pyridin-3-yl)thiazol-5-yl)phenyl)-3-hydroxy-1-methylpyrrolidin-2-one;
(R)-3-(3-(2-(5H-Pyrrolo[2,3-b]pyrazin-7-yl)thiazol-4-yl)phenyl)-2,3-dihydrofuro[3,2-b]pyridin-3-ol;
(R)-7-(3-(2-(5H-Pyrrolo[2,3-b]pyrazin-7-yl)thiazol-4-yl)phenyl)-6,7-dihydro-5H-pyrrolo[1,2-a]imidazol-7-ol;
(R)-7-(3-(4-(5H-Pyrrolo[2,3-b]pyrazin-7-yl)thiazol-2-yl)phenyl)-6,7-dihydro-5H-pyrrolo[1,2-a]imidazol-7-ol;
(S)-7-(3-(2-(1H-Pyrrolo[2,3-b]pyridin-3-yl)thiazol-4-yl)phenyl)-6,7-dihydro-5H-cyclopenta[b]pyridin-7-ol;
(S)-7-(3-(2-(5H-Pyrrolo[2,3-b]pyrazin-7-yl)thiazol-4-yl)phenyl)-7H-pyrrolo[1,2-a]imidazol-7-ol;
(R)-3-(3-(2-(5H-Pyrrolo[2,3-b]pyrazin-7-yl)thiazol-4-yl)phenyl)-3-hydroxy-1-methylazetidin-2-one;
(R)-3-(3-(2-(5H-Pyrrolo[2,3-b]pyrazin-7-yl)thiazol-4-yl)phenyl)-3-hydroxy-1-Methylpiperidin-2-one; and pharmaceutically acceptable salts thereof.

28-38. (canceled)

39. The compound of claim 1, wherein the compound is 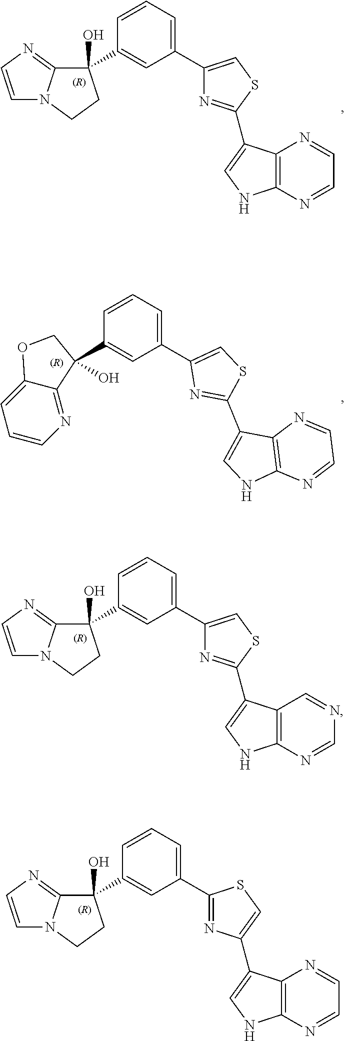 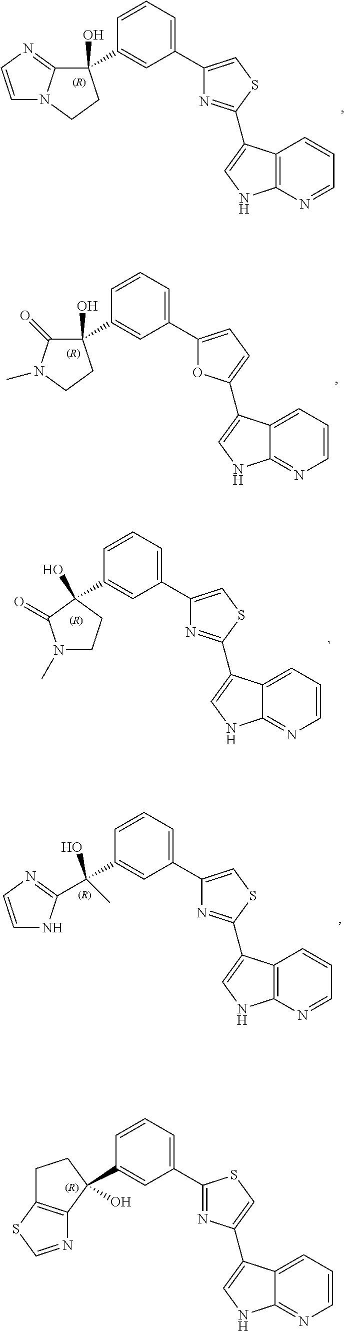
or a pharmaceutically acceptable salt thereof.

40. A pharmaceutical composition comprising a therapeutically effective amount of at least one compound or pharmaceutically acceptable salt thereof, as claimed in claim 1.

41. A pharmaceutical composition comprising a therapeutically effective amount of at least one compound or pharmaceutically acceptable salt thereof, as claimed in claim 26.

42. (canceled)

43. A method of treating a subject suffering from or diagnosed with a disease, disorder, or medical condition mediated by NIK activity, comprising administering to a subject in need of such treatment a therapeutically effective amount of at least one compound as claimed in claim 1 or a pharmaceutically acceptable salt thereof.

44. A method as claimed in claim 0 wherein the disease, disorder or medical condition is selected from the group consisting of cancer, inflammatory disorders, autoimmune disorders, immunodermatologic disorders and metabolic disorders.

45. A method as claimed in claim 0 wherein the disease, disorder or medical condition is selected from the group consisting of systemic lupus erythematosus, rheumatoid arthritis, Graft-Versus-Host Disease, transplant rejection, Sjogren's Syndrome, pemphigus vulgaris, palmoplantar pustulosis, hidradenitis suppurativa, obesity and diabetes.

46-48. (canceled)
